SEQUENCE LISTING (24 sequences)

SEQ ID NO: 1 - protein, 115 aa, Artificial Sequence
  Synthetic H1H7814N, HCVR
QVQLVESGGGVVQPGRSLRLSCVASGFTISSYGMNWVRQAPGKGLEWVAVISYDGGNKYSVDSVKGRFTISRDNSKNTLYLQMNSLRAEDSAVYYCARGRYFDLWGRGTLVTVSS

SEQ ID NO: 2 - protein, 8 aa, Artificial Sequence
  Synthetic H1H7814N, HCDR1
GFTISSYG

SEQ ID NO: 3 - protein, 8 aa, Artificial Sequence
  Synthetic H1H7814N, HCDR2
ISYDGGNK

SEQ ID NO: 4 - protein, 8 aa, Artificial Sequence
  Synthetic H1H7814N, HCDR3
ARGRYFDL

SEQ ID NO: 5 - protein, 107 aa, Artificial Sequence
  Synthetic H1H7814N, LCVR
DIQMTQSPSTLSASVGDRVTITCRASQSISSWLAWYQQKPGRAPNLLISKASSLKSGVPSRFSGSGSGTEFTLTVSSLQPDDFATYYCQQYYSYSYTFGQGTKLEIK

SEQ ID NO: 6 - protein, 6 aa, Artificial Sequence
  Synthetic H1H7814N, LCDR1
QSISSW

SEQ ID NO: 7 - protein, 3 aa, Artificial Sequence
  Synthetic H1H7814N, LCDR2
KAS

SEQ ID NO: 8 - protein, 9 aa, Artificial Sequence
  Synthetic H1H7814N, LCDR3
QQYYSYSYT

SEQ ID NO: 9 - protein, 115 aa, Artificial Sequence
  Synthetic H1H6958N2, HCVR
QVQLVESGGGVVQPGRSLRLSCGASGFTFRNYGMQWVRQGPGKGLEWVTLISFDGNDKYYADSVKGRFTISRDNSKNTLFLQMNSLRTEDTAVYYCARGGDFDYWGQGTLVTVSS

SEQ ID NO: 10 - protein, 8 aa, Artificial Sequence
  Synthetic H1H6958N2, HCDR1
GFTFRNYG

SEQ ID NO: 11 - protein, 8 aa, Artificial Sequence
  Synthetic H1H6958N2, HCDR2
ISFDGNDK

SEQ ID NO: 12 - protein, 8 aa, Artificial Sequence
  Synthetic H1H6958N2, HCDR3
ARGGDFDY

SEQ ID NO: 13 - protein, 108 aa, Artificial Sequence
  Synthetic H1H6958N2, LCVR
DIQMTQSPSSLSASVGDRVTITCRASQDIRKDLGWYQQKPGKAPKRLIYAASSLHSGVPSRFSGSGSGTEFTLTISSLQPEDFATYYCLQHNSYPMYTFGQGTKLEIK

SEQ ID NO: 14 - protein, 6 aa, Artificial Sequence
  Synthetic H1H6958N2, LCDR1
QDIRKD

SEQ ID NO: 15 - protein, 3 aa, Artificial Sequence
  Synthetic H1H6958N2, LCDR2
AAS

SEQ ID NO: 16 - protein, 10 aa, Artificial Sequence
  Synthetic H1H6958N2, LCDR3
LQHNSYPMYT

SEQ ID NO: 17 - protein, 267 aa, Artificial Sequence
  Synthetic hPRLR ecto-MMH
MHRPRRRGTRPPPLALLAALLLAARGADAQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDEQKLISEEDLGGEQKLISEEDLHHHHHH

SEQ ID NO: 18 - protein, 5 aa, Artificial Sequence
  Synthetic peptide linker
GGGGS

SEQ ID NO: 19 - protein, 5 aa, Artificial Sequence
  Synthetic peptide linker
GGGGG

SEQ ID NO: 20 - protein, 5 aa, Artificial Sequence
  Synthetic peptide linker
GGGGK

SEQ ID NO: 21 - protein, 4 aa, Artificial Sequence
  Synthetic peptide linker
GFGG

SEQ ID NO: 22 - protein, 4 aa, Artificial Sequence
  Synthetic peptide linker
GGGG

SEQ ID NO: 23 - protein, 4 aa, Artificial Sequence
  Synthetic peptide linker
GGFG

SEQ ID NO: 24 - protein, 4 aa, Artificial Sequence
  Synthetic
GGGE

(19) United States
(12) Patent Application Publication
(10) Pub. No.: US 2023/0069722 A1
(43) Pub. Date: Mar. 2, 2023

(54) TUBULYSINS AND PROTEIN-TUBULYSIN CONJUGATES

(71) Applicant: Regeneron Pharmaceuticals, Inc., Tarrytown, NY (US)

(72) Inventor: Amy HAN, Hockessin, DE (US)

(21) Appl. No.: 17/356,497

(22) Filed: Jun. 23, 2021

Related U.S. Application Data
(60) Provisional application No. 63/043,771, filed on Jun. 24, 2020.

(51) Int. Cl. A61K 47/68; C07D 417/12; A61P 35/00

(52) U.S. Cl. CPC A61K 47/6829; A61K 47/6889; A61P 35/00; A61K 47/6849; A61K 47/6871; C07D 417/12

(57) ABSTRACT

Provided herein are compounds, compositions, and methods for the treatment of diseases and disorders associated with cancer, including tubulysins and protein (e.g., antibody) drug conjugates thereof.

### CROSS REFERENCE

The present application claims the benefit of U.S. Provisional Application No. 63/043,771, filed Jun. 24, 2020, the contents of which are hereby incorporated by reference in their entirety.

### SEQUENCE LISTING

This application incorporates by reference the computer readable sequence listing in the file “2021-08-31 114581.00474_ST25.txt,” created Sep. 3, 2021, having 11 KB.

### FIELD

Provided herein are novel tubulysins and protein conjugates thereof, and methods for treating a variety of diseases, disorders, and conditions including administering the tubulysins, and protein conjugates thereof.

### BACKGROUND

While antibody-drug conjugates (ADCs) find increasing application in cancer treatment regimens, de novo or treatment-emergent resistance mechanisms could impair clinical benefit. Two resistance mechanisms that emerge under continuous ADC exposure in vitro include upregulation of transporters that confer multidrug resistance (MDR) and loss of cognate antigen expression. New technologies that circumvent these resistance mechanisms may serve to extend the utility of next generation ADCs.

The tubulysins, first isolated from myxobacterial culture broth, are a group of extremely potent tubulin polymerization inhibitors that rapidly disintegrate the cytoskeleton of dividing cells and induce apoptosis. Tubulysins are comprised of N-methyl-D-pipecolinic acid (Mep), L-isoleucine (Ile), and tubuvaline (Tuv), which contains an unusual N,O-acetal and a secondary alcohol or acetoxy group. Tubulysins A, B, C, G, and I contain the C-terminal tubutyrosine (Tut) γ-amino acid, while D, E, F, and H instead have tubuphenylalanine (Tup) at this position (Angew. Chem. Int. Ed. Engl. 43, 4888-4892).

Tubulysins have emerged as promising anticancer leads due to their powerful activity in drug-resistant cells through a validated mechanism of action. The average cell growth inhibitory activity outperforms that of well-known epothilones, vinblastines, and taxols by 10-fold to more than 1000-fold, including activity against multi-drug resistant carcinoma (Biochem. J. 2006, 396, 235-242; Nat. Prod. Rep. 2015, 32, 654-662). Tubulysins have extremely potent antiproliferative activity against cancer cells, including multidrug resistant KB-V1 cervix carcinoma cells. (Angew. Chem. Int. Ed. 2004, 43, 4888-4892; and Biochemical Journal 2006, 396, 235-242).

### SUMMARY

Provided herein are compounds useful, for example, in anti-cancer and anti-angiogenesis treatments.

In one embodiment, provided are compounds having the formula

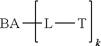

or a pharmaceutically acceptable salt thereof, wherein
BA is a binding agent;
L is a linker covalently bound to BA and to T;

### T is

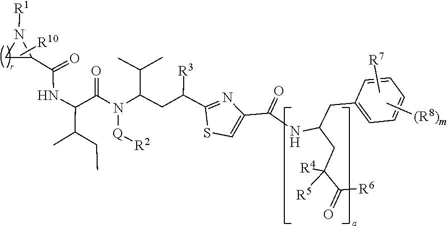

wherein
R¹ is a bond, hydrogen, C₁-C₁₀ alkyl, a first N-terminal amino acid residue, a first amino acid residue, —C₁-C₁₀ alkyl-NR^{3a}R^{3b}, or —C₁-C₁₀ alkyl-OH;
R³ is hydroxyl, —O—, —O—C₁-C₅ alkyl, —OC(O)C₁-C₅ alkyl, —OC(O)N(H)C₁-C₁₀ alkyl, —OC(O)N(H)C₁-C₁₀ alkyl-NR^{3a}R^{3b}, —NHC(O)C₁-C₅ alkyl, or —OC(O)N(H)(CH₂CH₂O)ₙC₁-C₁₀ alkyl-NR^{3a}R^{3b},

wherein R^{3a} and R^{3b} are independently in each instance, a bond, hydrogen, alkyl, alkenyl, alkynyl, cycloalkyl, aryl, heteroaryl, and acyl; wherein alkyl, alkenyl, alkynyl, cycloalkyl, aryl, heteroaryl, and acyl are optionally substituted;

R⁴ and R⁵ are, independently in each instance, hydrogen or C₁-C₅ alkyl;
R⁶ is —OH, —O—, —NHNH₂, —NHNH—, —NHSO₂(CH₂)ₐ₁-aryl-(CH₂)ₐ₂NR^{6a}R^{6b}, wherein aryl is substituted or unsubstituted; and

R^{6a} and R^{6b} are independently in each instance, a bond, hydrogen, alkyl, alkenyl, alkynyl, cycloalkyl, aryl, heteroaryl, and acyl; wherein alkyl, alkenyl, alkynyl, cycloalkyl, aryl, heteroaryl, and acyl are optionally substituted;

R⁷ is, independently in each instance, hydrogen, —OH, —O—, halogen, or —NR^{7a}R^{7b},

wherein R^{7a} and R^{7b} are, independently in each instance, a bond, hydrogen, alkyl, alkenyl, alkynyl, cycloalkyl, aryl, heteroaryl, acyl, —C(O)CH₂OH, —C(O)CH₂O—, a first N-terminal amino acid residue, a first amino acid residue, a first N-terminal peptide residue, a first peptide residue, —CH₂CH₂NH₂, and —CH₂CH₂NH—; wherein alkyl, alkenyl, alkynyl, cycloalkyl, aryl, heteroaryl, and acyl are optionally substituted;

R⁸ is, independently in each instance, hydrogen, —NHR⁹, or halogen,

wherein R⁹ is hydrogen, —C₁-C₅ alkyl, or —C(O)C₁-C₅ alkyl; and

m is one or two;

R¹⁰, when present, is —C₁-C₅ alkyl;
Q is —CH₂— or —O— wherein

R² is alkyl, alkylene, alkynyl, alkynylene, a regioisomeric triazole, a regioisomeric triazolylene;

wherein said regioisomeric triazole or regioisomeric triazolylene is unsubstituted or substituted with alkyl, alkenyl, alkynyl, cycloalkyl, aryl, heteroaryl, or acyl;
wherein n is an integer from one to ten;
wherein r is an integer from one to six;
wherein a, a1, and, a2 are, independently, zero or one; and
k is an integer from one to thirty;
wherein T is not compound IVa, IVa′, IVb, IVc, IVd, IVe, IVf, IVg, IVh, IVj, IVk, IVl, IVm, IVn, IVo, IVp, IVq, IVr, IVs, IVt, IVu, IVvA, IVvB, IVw, IVx, IVy, Va, Va′, Vb, Vc, Vd, Ve, Vf, Vg, Vh, Vi, Vj, Vk, VIa, IVb, VIc, VId, VIe, VIf, VIg, VIh, Vi, VIi, VII, VIII, IX, X, D-5a, and D-5c, or a pharmaceutically acceptable salt thereof, covalently bound to L.

In one embodiment, provided are compounds having the structure of Formula I

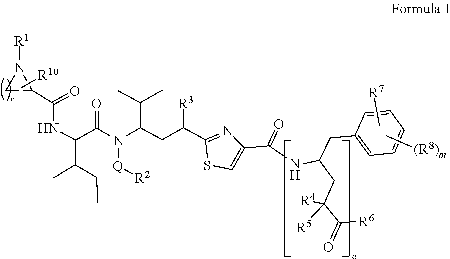

or a pharmaceutically acceptable salt thereof, wherein
R¹ is hydrogen, C₁-C₁₀ alkyl, a first N-terminal amino acid residue, —C₁-C₁₀ alkyl-NR^{3a}R^{3b}, or —C₁-C₁₀ alkyl-OH;
R³ is hydroxyl, —O—C₁-C₅ alkyl, —OC(O)C₁-C₅ alkyl, —OC(O)N(H)C₁-C₁₀ alkyl, —OC(O)N(H)C₁-C₁₀ alkyl-NR^{3a}R^{3b}, —NHC(O)C₁-C₅ alkyl, or —OC(O)N(H)(CH₂CH₂O)ₙC₁-C₁₀ alkyl-NR^{3a}R^{3b},

wherein R^{3a} and R^{3b} are independently in each instance, hydrogen, alkyl, alkenyl, alkynyl, cycloalkyl, aryl, heteroaryl, and acyl; wherein alkyl, alkenyl, alkynyl, cycloalkyl, aryl, heteroaryl, and acyl are optionally substituted;

R⁴ and R⁵ are, independently in each instance, hydrogen or C₁-C₅ alkyl;
R⁶ is —OH, —NHNH₂, —NHSO₂(CH₂)ₐ₁-aryl-(CH₂)ₐ₂NR^{6a}R^{6b},

wherein aryl is substituted or unsubstituted; and

R^{6a} and R^{6b} are independently in each instance, hydrogen, alkyl, alkenyl, alkynyl, cycloalkyl, aryl, heteroaryl, and acyl; wherein alkyl, alkenyl, alkynyl, cycloalkyl, aryl, heteroaryl, and acyl are optionally substituted;

R⁷ is, independently in each instance, hydrogen, —OH, halogen, or —NR^{7a}R^{7b},

wherein R^{7a} and R^{7b} are, independently in each instance, hydrogen, alkyl, alkenyl, alkynyl, cycloalkyl, aryl, heteroaryl, acyl, —C(O)CH₂OH, a first N-terminal amino acid residue, a first N-terminal peptide residue, and —CH₂CH₂NH₂; wherein alkyl, alkenyl, alkynyl, cycloalkyl, aryl, heteroaryl, and acyl are optionally substituted;

R⁸ is, independently in each instance, hydrogen, —NHR⁹, or halogen,

wherein R⁹ is hydrogen, —C₁-C₅ alkyl, or —C(O)C₁-C₅ alkyl; and

m is one or two;

R¹⁰, when present, is —C₁-C₅ alkyl;
Q is —CH₂— or —O— wherein

R² is alkyl, alkynyl, or a regioisomeric triazole;

wherein said regioisomeric triazole is unsubstituted or substituted with alkyl, alkenyl, alkynyl, cycloalkyl, aryl, heteroaryl, and acyl;
wherein n is an integer from one to ten;
wherein r is an integer from one to six;
wherein a, a1, and, a2 are, independently, zero or one; and
wherein T is not compound IVa, IVa′, IVb, IVc, IVd, IVe, IVf, IVg, IVh, IVj, IVk, IVl, IVm, IVn, IVo, IVp, IVq, IVr, IVs, IVt, IVu, IVvA, IVvB, IVw, IVx, IVy, Va, Va′, Vb, Vc, Vd, Ve, Vf, Vg, Vh, Vi, Vj, Vk, VIa, IVb, VIc, VId, VIe, VIf, VIg, VIh, Vi, VIi, VII, VIII, IX, X, D-5a, D-5c, Tubulysin A-I, U-X, or Z, Pretubulysin D, or N¹⁴-desacetoxytubulysin H.

In another embodiment, provided is a method for treating tumors that express an antigen selected from the group consisting of PRLR and STEAP2.

In another embodiment, provided is a linker-payload having the formula

L-T

or a pharmaceutically acceptable salt thereof, wherein
L is a linker covalently bound to T;

### T is

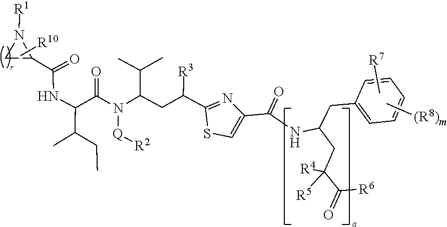

wherein
R¹ is a bond, hydrogen, C₁-C₁₀ alkyl, a first N-terminal amino acid residue, a first amino acid residue, —C₁-C₁₀ alkyl-NR^{3a}R^{3b}, or —C₁-C₁₀ alkyl-OH;
R³ is hydroxyl, —O—, —O—C₁-C₅ alkyl, —OC(O)C₁-C₅ alkyl, —OC(O)N(H)C₁-C₁₀ alkyl, —OC(O)N(H)C₁-C₁₀ alkyl-NR^{3a}R^{3b}, —NHC(O)C₁-C₅ alkyl, or —OC(O)N(H)(CH₂CH₂O)ₙC₁-C₁₀ alkyl-NR^{3a}R^{3b},

wherein R^{3a} and R^{3b} are independently in each instance, a bond, hydrogen, alkyl, alkenyl, alkynyl, cycloalkyl, aryl, heteroaryl, and acyl; wherein alkyl, alkenyl, alkynyl, cycloalkyl, aryl, heteroaryl, and acyl are optionally substituted;

R⁴ and R⁵ are, independently in each instance, hydrogen or C₁-C₅ alkyl;
R⁶ is —OH, —O—, —NHNH₂, —NHNH—, —NHSO₂(CH₂)ₐ₁-aryl-(CH₂)ₐ₂NR^{6a}R^{6b},

wherein aryl is substituted or unsubstituted; and

R^{6a} and R^{6b} are independently in each instance, a bond, hydrogen, alkyl, alkenyl, alkynyl, cycloalkyl, aryl, heteroaryl, and acyl; wherein alkyl, alkenyl, alkynyl, cycloalkyl, aryl, heteroaryl, and acyl are optionally substituted;

R⁷ is, independently in each instance, hydrogen, —OH, —O—, halogen, or —NR^{7a}R^{7b},

wherein R^{7a} and R^{7b} are, independently in each instance, a bond, hydrogen, alkyl, alkenyl, alkynyl, cycloalkyl, aryl, heteroaryl, acyl, —C(O)CH₂OH, —C(O)CH₂O—, a first N-terminal amino acid residue, a first amino acid residue, a first N-terminal peptide residue, a first peptide residue, —CH₂CH₂NH₂, and —CH₂CH₂NH—; wherein alkyl, alkenyl, alkynyl, cycloalkyl, aryl, heteroaryl, and acyl are optionally substituted;

R⁸ is, independently in each instance, hydrogen, —NHR⁹, or halogen,

wherein R⁹ is hydrogen, —C₁-C₅ alkyl, or —C(O)C₁-C₅ alkyl; and

m is one or two;

R¹⁰, when present, is —C₁-C₅ alkyl;
Q is —CH₂— or —O— wherein

R² is alkyl, alkylene, alkynyl, alkynylene, a regioisomeric triazole, a regioisomeric triazolylene;

wherein said regioisomeric triazole or regioisomeric triazolylene is unsubstituted or substituted with alkyl, alkenyl, alkynyl, cycloalkyl, aryl, heteroaryl, or acyl;
wherein n is an integer from one to ten;
wherein r is an integer from one to six;
wherein a, a1, and, a2 are, independently, zero or one; and
wherein the linker-payload is not LP1-IVa, LP2-Va, LP3-IVd, LP4-Ve, LP5-IVd, LP6-Vb, LP7-IVd, LP9-IVvB, LP10-VIh, LP11-IVvB, LP12-VIi, LP13-Ve, LP14-Ve, LP15-VIh, LP16-Ve, LP17-Ve, LP18-Ve, LP19-Ve, LP20-Ve, LP21-Ve, LP22-Ve, LP23-Vb, LP24-Vb, LP25-Ve, and LP26-Ve, or a pharmaceutically acceptable salt thereof.

In another embodiment, set forth herein is an antibody-drug conjugate including an antibody, or antigen-binding fragment thereof, wherein said antibody or antigen-binding fragment thereof is conjugated to a compound as described herein.

In another embodiment, set forth herein are methods for making the compounds, linker-payloads, or antibody-drug conjugates, and compositions described herein.

### BRIEF DESCRIPTIONS OF THE DRAWING

FIGS. 1-11, 12A, 12B, 13A, 13B, 14, 15A, 15B, 15C, and 16 show synthetic chemistry schemes for tubulyisin payloads, and tubulysin linker-payloads, wherein each are capable of conjugation to or conjugated to an antibody or antigen-binding fragment thereof.

### DESCRIPTION OF EXEMPLARY EMBODIMENTS

Provided herein are compounds, compositions, and methods useful for treating for example, cancer in a subject.

### Definitions

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term provided herein, these Definitions prevail unless stated otherwise.

As used herein, “alkyl” refers to a monovalent and saturated hydrocarbon radical moiety. Alkyl is optionally substituted and can be linear, branched, or cyclic, i.e., cycloalkyl. Alkyl includes, but is not limited to, those radicals having 1-20 carbon atoms, i.e., C₁₋₂₀ alkyl; 1-12 carbon atoms, i.e., C₁₋₁₂ alkyl; 1-10 carbon atoms, i.e., C₁₋₁₀ alkyl; 1-8 carbon atoms, i.e., C₁₋₈ alkyl; 5-10 carbon atoms, i.e., C₅₋₁₀ alkyl; 1-5 carbon atoms, i.e., C₁₋₅ alkyl; 1-6 carbon atoms, i.e., C₁₋₆ alkyl; and 1-3 carbon atoms, i.e., C₁₋₃ alkyl. Examples of alkyl moieties include, but are not limited to, methyl, ethyl, n-propyl, i-propyl, n-butyl, s-butyl, t-butyl, i-butyl, a pentyl moiety, a hexyl moiety, cyclopropyl, cyclobutyl, cyclopentyl, and cyclohexyl. A pentyl moiety includes, but is not limited to, n-pentyl and i-pentyl. A hexyl moiety includes, but is not limited to, n-hexyl.

As used herein, “alkylene” refers to a divalent alkyl group. Unless specified otherwise, alkylene includes, but is not limited to, 1-20 carbon atoms. The alkylene group is optionally substituted as described herein for alkyl. In some embodiments, alkylene is unsubstituted.

Designation of an amino acid or amino acid residue without specifying its stereochemistry is intended to encompass the L-form of the amino acid, the D-form of the amino acid, or a racemic mixture thereof.

As used herein, “haloalkyl” refers to alkyl, as defined above, wherein the alkyl includes at least one substituent selected from a halogen, for example, fluorine (F), chlorine (C₁), bromine (Br), or iodine (I). Examples of haloalkyl include, but are not limited to, —CF₃, —CH₂CF₃, —CCl₂F, and —CCl₃.

As used herein, “alkenyl” refers to a monovalent hydrocarbon radical moiety containing at least two carbon atoms and one or more non-aromatic carbon-carbon double bonds. Alkenyl is optionally substituted and can be linear, branched, or cyclic. Alkenyl includes, but is not limited to, those radicals having 2-20 carbon atoms, i.e., C₂-20 alkenyl; 2-12 carbon atoms, i.e., C₂₋₁₂ alkenyl; 2-8 carbon atoms, i.e., C₂₋ₛ alkenyl; 2-6 carbon atoms, i.e., C₂₋₆ alkenyl; and 2-4 carbon atoms, i.e., C₂₄ alkenyl. Examples of alkenyl moieties include, but are not limited to, vinyl, propenyl, butenyl, and cyclohexenyl.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₄ alkynyl. Examples of alkynyl moieties include, but are not limited to ethynyl, propynyl, and butynyl.

As used herein, “alkoxy” refers to a monovalent and saturated hydrocarbon radical moiety wherein the hydrocarbon includes a single bond to an oxygen atom and wherein the radical is localized on the oxygen atom, e.g., CH₃CH₂—O. for ethoxy. Alkoxy substituents bond to the compound which they substitute through this oxygen atom of the alkoxy substituent. Alkoxy is optionally substituted and can be linear, branched, or cyclic, i.e., cycloalkoxy. Alkoxy includes, but is not limited to, those having 1-20 carbon atoms, i.e., C₁₋₂₀ alkoxy; 1-12 carbon atoms, i.e., C₁₋₁₂ alkoxy; 1-8 carbon atoms, i.e., C₁₋₈ alkoxy; 1-6 carbon atoms, i.e., C₁₋₆ alkoxy; and 1-3 carbon atoms, i.e., C₁₋₃ alkoxy. Examples of alkoxy moieties include, but are not limited to, methoxy, ethoxy, n-propoxy, i-propoxy, n-butoxy, s-butoxy, t-butoxy, i-butoxy, a pentoxy moiety, a hexoxy moiety, cyclopropoxy, cyclobutoxy, cyclopentoxy, and cyclohexoxy.

As used herein, “haloalkoxy” refers to alkoxy, as defined above, wherein the alkoxy includes at least one substituent selected from a halogen, e.g., F, C₁, Br, or I.

As used herein, “aryl” refers to a monovalent moiety that is a radical of an aromatic compound wherein the ring atoms are carbon atoms. Aryl is optionally substituted and can be monocyclic or polycyclic, e.g., bicyclic or tricyclic. Examples of aryl moieties include, but are not limited to, those having 6 to 20 ring carbon atoms, i.e., C₆₋₂₀ aryl; 6 to 15 ring carbon atoms, i.e., C₆₋₁₅ aryl, and 6 to 10 ring carbon atoms, i.e., C₆₋₁₀ aryl. Examples of aryl moieties include, but are limited to, phenyl, naphthyl, fluorenyl, azulenyl, anthryl, phenanthryl, and pyrenyl.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g.,

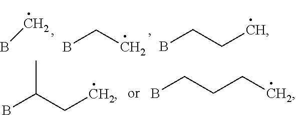

wherein B is an aromatic moiety, e.g., aryl or phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g.,

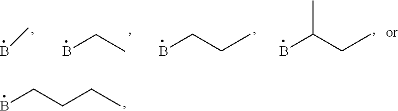

wherein B is an aromatic moiety, e.g., phenyl. Alkylaryl is optionally substituted,

i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g.,

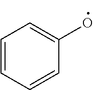

for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy, and 6 to 10 ring carbon atoms, i.e., C₆₋₁₀ aryloxy. Examples of aryloxy moieties include, but are not limited to phenoxy, naphthoxy, and anthroxy.

As used herein, “arylene” refers to a divalent moiety of an aromatic compound wherein the ring atoms are only carbon atoms. Arylene is optionally substituted and can be monocyclic or polycyclic, e.g., bicyclic or tricyclic. Examples of arylene moieties include, but are not limited to those having 6 to 20 ring carbon atoms, i.e., C₆₋₂₀ arylene; 6 to 15 ring carbon atoms, i.e., C₆₋₁₅ arylene, and 6 to 10 ring carbon atoms, i.e., C₆₋₁₀ arylene.

As used herein, “heteroalkyl” refers to an alkyl in which one or more carbon atoms are replaced by heteroatoms. As used herein, “heteroalkenyl” refers to an alkenyl in which one or more carbon atoms are replaced by heteroatoms. As used herein, “heteroalkynyl” refers to an alkynyl in which one or more carbon atoms are replaced by heteroatoms. Suitable heteroatoms include, but are not limited to, nitrogen, oxygen, and sulfur atoms. Heteroalkyl, heteroalkenyl, and heteroalkynyl are optionally substituted. Examples of heteroalkyl moieties include, but are not limited to, aminoalkyl, sulfonylalkyl, and sulfinylalkyl. Examples of heteroalkyl moieties also include, but are not limited to, methylamino, methylsulfonyl, and methylsulfinyl.

As used herein, “heteroaryl” refers to a monovalent moiety that is a radical of an aromatic compound wherein the ring atoms contain carbon atoms and at least one oxygen, sulfur, nitrogen, or phosphorus atom. Examples of heteroaryl moieties include, but are not limited to those having 5 to 20 ring atoms; 5 to 15 ring atoms; and 5 to 10 ring atoms. Heteroaryl is optionally substituted.

As used herein, “heteroarylene” refers to a divalent heteroaryl in which one or more ring atoms of the aromatic ring are replaced with an oxygen, sulfur, nitrogen, or phosphorus atom. Heteroarylene is optionally substituted.

As used herein, “heterocycloalkyl” refers to a cycloalkyl in which one or more carbon atoms are replaced by heteroatoms. Suitable heteroatoms include, but are not limited to, nitrogen, oxygen, and sulfur atoms. Heterocycloalkyl is optionally substituted. Examples of heterocycloalkyl moieties include, but are not limited to, morpholinyl, piperidinyl, tetrahydropyranyl, pyrrolidinyl, imidazolidinyl, oxazolidinyl, thiazolidinyl, dioxolanyl, dithiolanyl, oxanyl, or thianyl.

As used herein, “Lewis acid” refers to a molecule or ion that accepts an electron lone pair. The Lewis acids used in the methods described herein are those other than protons. Lewis acids include, but are not limited to, non-metal acids, metal acids, hard Lewis acids, and soft Lewis acids. Lewis acids include, but are not limited to, Lewis acids of aluminum, boron, iron, tin, titanium, magnesium, copper, antimony, phosphorus, silver, ytterbium, scandium, nickel, and zinc. Illustrative Lewis acids include, but are not limited to, AlBr₃, AlCl₃, BCl₃, boron trichloride methyl sulfide, BF₃, boron trifluoride methyl etherate, boron trifluoride methyl sulfide, boron trifluoride tetrahydrofuran, dicyclohexylboron trifluoromethanesulfonate, iron (III) bromide, iron (III) chloride, tin (IV) chloride, titanium (IV) chloride, titanium (IV) isopropoxide, Cu(OTf)₂, CuCl₂, CuBr₂, zinc chloride, alkylaluminum halides (RₙAlX₃₋ₙ, wherein R is hydrocarbyl), Zn(OTf)₂, ZnCl₂, Yb(OTf)₃, Sc(OTf)₃, MgBr₂, NiCl₂, Sn(OTf)₂, Ni(OTf)₂, and Mg(OTf)₂.

As used herein, “N-containing heterocycloalkyl,” refers to a cycloalkyl in which one or more carbon atoms are replaced by heteroatoms and wherein at least one replacing heteroatom is a nitrogen atom. Suitable heteroatoms in addition to nitrogen, include, but are not limited to, oxygen and sulfur atoms. N-containing heterocycloalkyl is optionally substituted. Examples of N-containing heterocycloalkyl moieties include, but are not limited to, morpholinyl, piperidinyl, pyrrolidinyl, imidazolidinyl, oxazolidinyl, or thiazolidinyl.

As used herein, “optionally substituted,” when used to describe a radical moiety, for example, optionally substituted alkyl, means that such moiety is optionally bonded to one or more substituents. Examples of such substituents include, but are not limited to, halo, cyano, nitro, amino, hydroxyl, optionally substituted haloalkyl, aminoalkyl, hydroxyalkyl, azido, epoxy, optionally substituted heteroaryl, optionally substituted heterocycloalkyl,

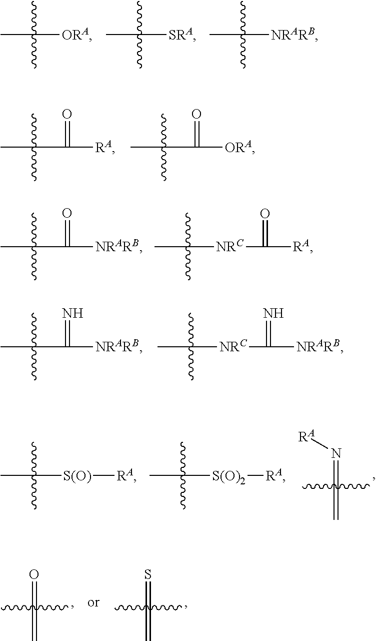

wherein R^{A}, R^{B}, and R^{C} are, independently at each occurrence, a hydrogen atom, alkyl, alkenyl, alkynyl, aryl, alkylaryl, arylalkyl, heteroalkyl, heteroaryl, or heterocycloalkyl, or R^{A} and R^{B} together with the atoms to which they are bonded, form a saturated or unsaturated carbocyclic ring, wherein the ring is optionally substituted, and wherein one or more ring atoms is optionally replaced with a heteroatom. In certain embodiments, when a radical moiety is optionally substituted with an optionally substituted heteroaryl, optionally substituted heterocycloalkyl, or optionally substituted saturated or unsaturated carbocyclic ring, the substituents on the optionally substituted heteroaryl, optionally substituted heterocycloalkyl, or optionally substituted saturated or unsaturated carbocyclic ring, if they are substituted, are not substituted with substituents which are further optionally substituted with additional substituents. In some embodiments, when a group described herein is optionally substituted, the substituent bonded to the group is unsubstituted unless otherwise specified.

As used herein, “binding agent” refers to any molecule, e.g., protein, antibody, or fragment thereof, capable of binding with specificity to a given binding partner, e.g., antigen.

As used herein, “linker” refers to a divalent, trivalent, or multivalent moiety that covalently links, or is capable of covalently linking (e.g., via a reactive group at one terminus; and, in certain embodiments, an amino acid and/or a spacer at another terminus), the binding agent to one or more compounds described herein, for instance, payload compounds, enhancement agents, and/or prodrug payload compounds. As used herein, “payloads” refer to tubulysins or tubulysin derivatives. As used herein, “prodrug payload compounds” or “prodrugs” refer to payloads that terminate with one or more amino acid residues, or another chemical residue, as described elsewhere herein. Thus, in certain embodiments, the linker can ultimately be cleaved to release payload compounds in the form of tubulysin derivatives. In other embodiments, the linker can ultimately be cleaved to release a prodrug payload compound in the form of a tubulysin derivative that retains one or more terminal amino acid residues. Such a prodrug payload compound can be further processed via accepted biological processes (e.g., amide bond hydrolysis) that ultimately produce payload compounds in the form of tubulysin payload compounds without terminal amino acid residues.

As used herein, “amide synthesis conditions” refers to reaction conditions suitable to effect the formation of an amide, e.g., by the reaction of a carboxylic acid, activated carboxylic acid, or acyl halide with an amine. In some examples, amide synthesis conditions refers to reaction conditions suitable to effect the formation of an amide bond between a carboxylic acid and an amine. In some of these examples, the carboxylic acid is first converted to an activated carboxylic acid before the activated carboxylic acid reacts with an amine to form an amide. Suitable conditions to effect the formation of an amide include, but are not limited to, those utilizing reagents to effect the reaction between a carboxylic acid and an amine, including, but not limited to, dicyclohexylcarbodiimide (DCC), diisopropylcarbodiimide (DIC), (benzotriazol-1-yloxy)tris(dimethylamino)phosphonium hexafluorophosphate (BOP), (benzotriazol-1-yloxy)tripyrrolidinophosphonium hexafluorophosphate (PyBOP), (7-azabenzotriazol-1-yloxy)tripyrrolidinophosphonium hexafluorophosphate (PyAOP), bromotripyrrolidinophosphonium hexafluorophosphate (PyBrOP), O-(benzotriazol-1-yl)-N,N,N′,N′-tetramethyluronium hexafluorophosphate (HBTU), 0-(benzotriazol-1-yl)-N,N,N′,N′-tetramethyluronium tetrafluoroborate (TBTU), 1-[Bis(dimethylamino)methylene]-1H-1,2,3-triazolo[4,5-b]pyridinium 3-oxide hexafluorophosphate (HATU), N-ethoxycarbonyl-2-ethoxy-1,2-dihydroquinoline (EEDQ), N-ethyl-N′-(3-dimethylaminopropyl)carbodiimide (EDC), 2-chloro-1,3-dimethylimidazolidinium hexafluorophosphate (CIP), 2-chloro-4,6-dimethoxy-1,3,5-triazine (CDMT), and carbonyldiimidazole (CDI). In some examples, a carboxylic acid is first converted to an activated carboxylic ester before treating the activated carboxylic ester with an amine to form an amide bond. In certain embodiments, the carboxylic acid is treated with a reagent. The reagent activates the carboxylic acid by deprotonating the carboxylic acid and then forming a product complex with the deprotonated carboxylic acid as a result of nucleophilic attack by the deprotonated carboxylic acid onto the protonated reagent. The activated carboxylic esters for certain carboxylic acids are subsequently more susceptible to nucleophilic attack by an amine than the carboxylic acid is before it is activated. This results in amide bond formation. As such, the carboxylic acid is described as activated. Exemplary reagents include DCC and DIC.

As used herein, “regioisomer,” “regioisomers,” or “mixture of regioisomers” refers to the product(s) of 1,3-cycloadditions or strain-promoted alkyne-azide cycloadditions (SPAACs)—otherwise known as click reactions—that derive from suitable azides (e.g., —N₃, or -PEG-N₃ derivitized antibodies) treated with suitable alkynes. In certain embodiments, for example, regioisomers and mixtures of regioisomers are characterized by the click reaction products shown below:

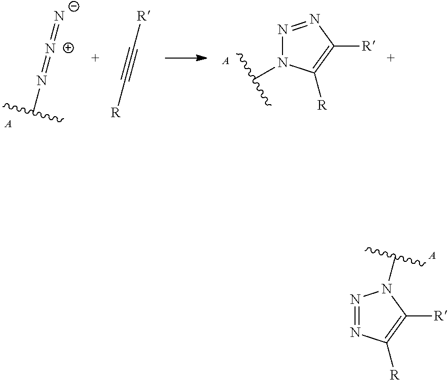

In certain embodiments, more than one suitable azide and more than one suitable alkyne can be utilized within a synthetic scheme en route to a product, where each pair of azide-alkyne can participate in one or more independent click reactions to generate a mixture of regioisomeric click reaction products. For example, a person of skill will recognize that a first suitable azide may independently react with a first suitable alkyne, and a second suitable azide may independently react with a second suitable alkyne, en route to a product, resulting in the generation of four possible click reaction regioisomers or a mixture of the four possible click reaction regioisomers.

As used herein, the term “residue” refers to the chemical moiety within a compound that remains after a chemical reaction. For example, the term “amino acid residue,” “N-alkyl amino acid residue,” or “N-terminal amino acid residue” refers to the product of an amide coupling or peptide coupling of an amino acid, N-alkyl amino acid, or N-terminal amino acid” to a suitable coupling partner; wherein, for example, a water molecule is expelled after the amide or peptide coupling of the amino acid or the N-alkylamino acid, resulting in the product having the amino acid residue, N-alkyl amino acid residue, or N-terminal amino acid residue, incorporated therein. The term “amino acid” refers to naturally occurring and synthetic α, β, γ, or δ amino acids, and includes, but is not limited to, amino acids found in proteins, viz., glycine, alanine, valine, leucine, isoleucine, methionine, phenylalanine, tryptophan, proline, serine, threonine, cysteine, tyrosine, asparagine, glutamine, aspartate, glutamate, lysine, arginine, and histidine. In certain embodiments, the amino acid is in the L-configuration. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleuccinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. The term “amino acid derivative” refers to a group derivable from a naturally or non-naturally occurring amino acid, as described and exemplified herein. Amino acid derivatives are apparent to those of skill in the art and include, but are not limited to, ester, amino alcohol, amino aldehyde, amino lactone, and N-methyl derivatives of naturally and non-naturally occurring amino acids. In certain embodiments, an amino acid residue is

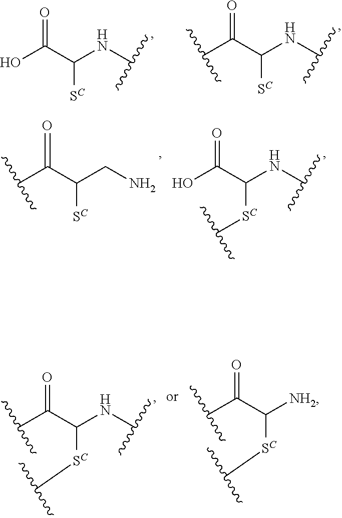

wherein S^{c} is a side chain of a naturally occurring or non-naturally occurring amino acid or a bond (e.g., hydrogen, as in glycine; —CH₂OH as in serine; —CH₂SH as in cysteine; —CH₂CH₂CH₂CH₂NH₂ as in lysine; —CH₂CH₂COOH as in glutamic acid; —CH₂CH₂C(O)NH₂ as in glutamine; or —CH₂C₆H₅OH as in tyrosine; and the like); and [symbol] represents the bonding to another chemical entity, including, but not limited to, another amino acid residue or N-alkyl amino acid residue resulting in a peptide or peptide residue. In certain embodiments, S^{c} is selected from the group consisting of hydrogen, alkyl, heteroalkyl, arylalkyl, and heteroarylalkyl.

As used herein, “therapeutically effective amount” refers to an amount (e.g., of a compound) that is sufficient to provide a therapeutic benefit to a patient in the treatment or management of a disease or disorder, or to delay or minimize one or more symptoms associated with the disease or disorder.

As used herein, “constitutional isomers” refers to compounds that have the same molecular formula, but different chemical structures resulting from the way the atoms are arranged. Exemplary constitutional isomers include n-propyl and isopropyl; n-butyl, sec-butyl, and tert-butyl; and n-pentyl, isopentyl, and neopentyl, and the like.

Certain groups, moieties, substituents, and atoms are depicted with a wiggly line that intersects a bond or bonds to indicate the atom through which the groups, moieties, substituents, atoms are bonded. For example, a phenyl group that is substituted with a propyl group depicted as:

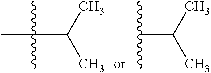

has the following structure:

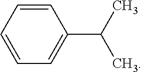

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which this disclosure pertains. For example, the group,

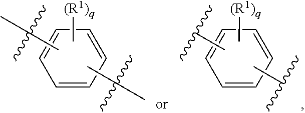

wherein subscript q is an integer from zero to four and in which the positions of substituent R¹ are described generically, i.e., not directly attached to any vertex of the bond line structure, i.e., specific ring carbon atom, includes the following, non-limiting examples of groups in which the substituent R¹ is bonded to a specific ring carbon atom:

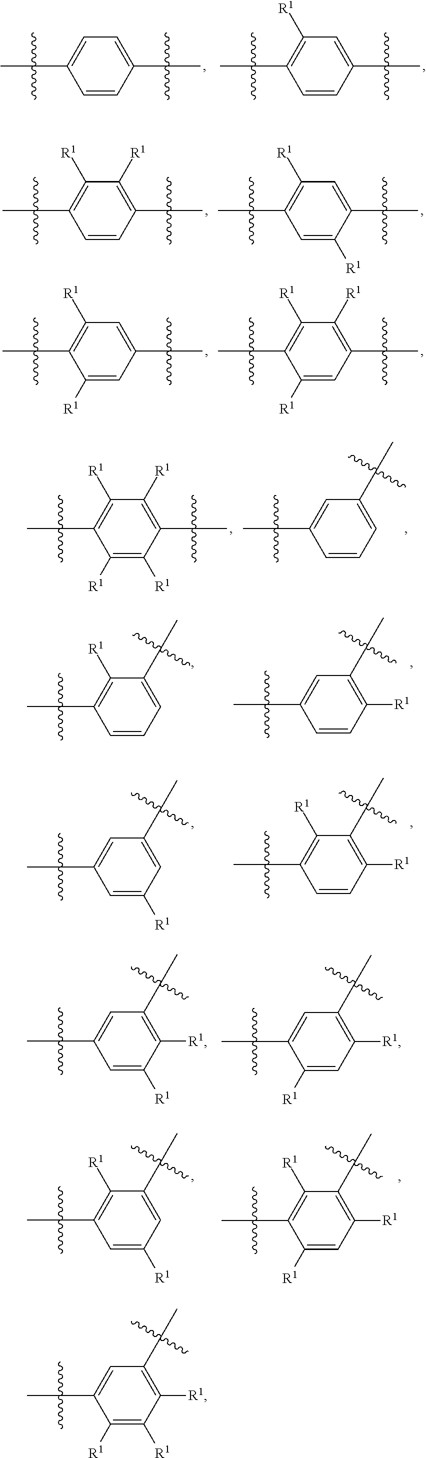

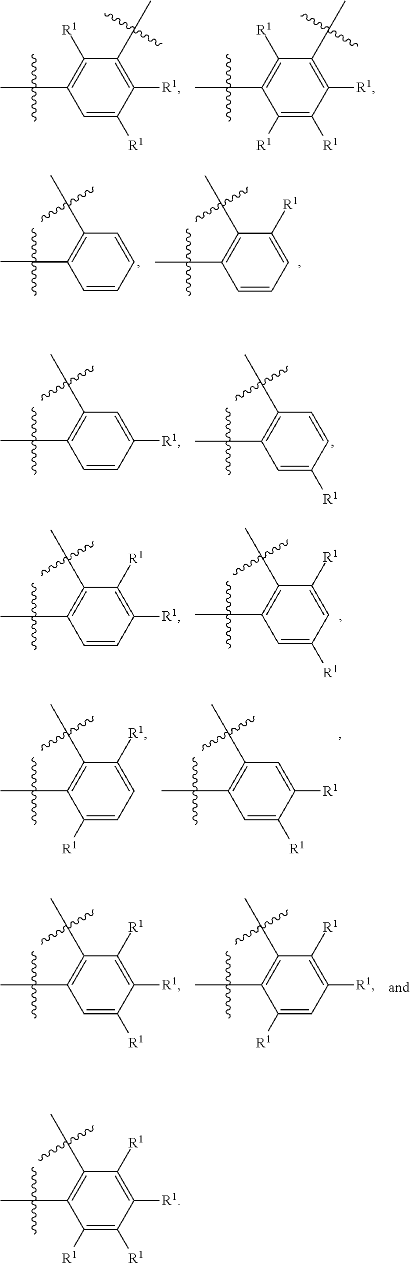

As used herein, the phrase “reactive linker,” or the abbreviation “RL” refers to a monovalent group that includes a reactive group (“RG”) and spacer group (“SP”), depicted, for example, as

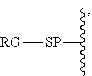

wherein RG is the reactive group and SP is the spacer group. As described herein, a reactive linker may include more than one reactive group and more than one spacer group. The spacer group is any divalent moiety that bridges the reactive group to another group, such as a payload or prodrug payload. The reactive linkers (RLs), together with the payloads or prodrug payloads to which they are bonded, provide intermediates (“linker-payloads” or LPs; or linker-prodrug payloads) useful as synthetic precursors for the preparation of the antibody conjugates described herein. The reactive linker includes a reactive group, which is a functional group or moiety that is capable of reacting with a reactive portion of another group, for instance, an antibody or antigen-binding fragment thereof, modified antibody or antigen-binding fragment thereof, transglutaminase-modified antibody or antigen-binding fragment thereof, or an enhancement group. The moiety resulting from the reaction of the reactive group with the antibody or antigen-binding fragment thereof, modified antibody or antigen-binding fragment thereof, or transglutaminase-modified antibody or antigen-binding fragment thereof, together with the linking group, include the “binding agent linker” (“BL”) portion of the conjugate, described herein. In certain embodiments, the “reactive group” is a functional group or moiety (e.g., maleimide or N-hydroxysuccinimide (NHS) ester) that reacts with a cysteine or lysine residue of an antibody or antigen-binding fragment thereof. In certain embodiments, the “reactive group” is a functional group or moiety that is capable of undergoing a click chemistry reaction (see, e.g., click chemistry, Huisgen Proc. Chem. Soc. 1961, Wang et al. J. Am. Chem. Soc. 2003, and Agard et al. J. Am. Chem. Soc. 2004). In some embodiments of said click chemistry reaction, the reactive group is an alkyne that is capable of undergoing a 1,3-cycloaddition reaction with an azide. Such suitable reactive groups include, but are not limited to, strained alkynes, e.g., those suitable for strain-promoted alkyne-azide cycloadditions (SPAAC), cycloalkynes, e.g., cyclooctynes, benzannulated alkynes, and alkynes capable of undergoing 1,3-cycloaddition reactions with alkynes in the absence of copper catalysts. Suitable alkynes also include, but are not limited to, dibenzoazacyclooctyne or

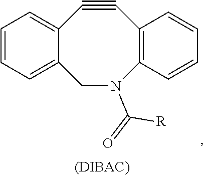

dibenzocyclooctyne or

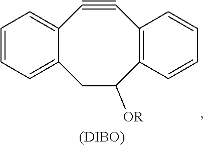

biarylazacyclooctynone or

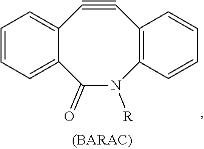

difluorinated cyclooctyne or

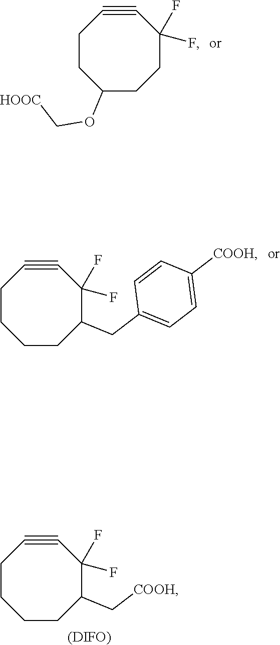

substituted, e.g., fluorinated alkynes, aza-cycloalkynes, bicycle[6.1.0]nonyne or

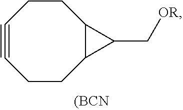

where R is alkyl, alkoxy, or acyl), and derivatives thereof. Particularly useful alkynes include

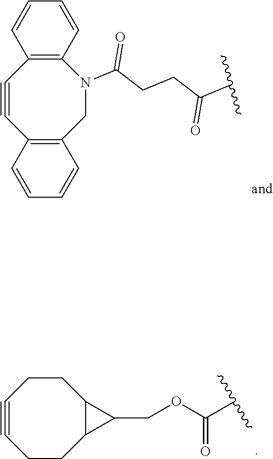 [SMILES: *C(=O)CCC(=O)N1Cc2ccccc2C#Cc2ccccc21 ; *C(=O)OCC1C2CCC#CCCC21]

Linker-payloads or linker-prodrug payloads including such reactive groups are useful for conjugating antibodies that have been functionalized with azido groups. As used herein, a “transglutaminase-modified antibody or antigen-binding fragment thereof” refers to an antibody or antigen-binding fragment thereof having one or more glutamine (Gln or Q) residues capable of reaction with a compound bearing a primary or secondary amino functional group in the presence of the enzyme transglutaminase. Such transglutaminase-modified antibodies or antigen-binding fragments thereof include antibodies or antigen-binding fragments thereof functionalized with azido-polyethylene glycol groups via transglutaminase-mediated coupling of an antibody or antigen-binding fragment thereof with a primary amine bearing the azido-polyethylene glycol moiety. In certain embodiments, such a transglutaminase-modified antibody or antigen-binding fragment thereof is derived by treating an antibody or antigen-binding fragment thereof having at least one glutamine residue, e.g., heavy chain Gln295, with a compound bearing an amino group and an azide group, in the presence of the enzyme transglutaminase, as further described elsewhere herein.

In some examples, the reactive group is an alkyne, e.g.,

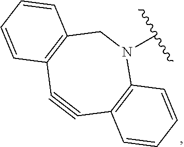 [SMILES: *N1Cc2ccccc2C#Cc2ccccc21]

which can react via click chemistry with an azide, e.g.,

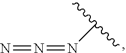

to form a click chemistry product, e.g.,

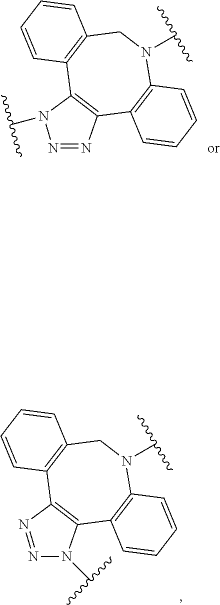 [SMILES: *N1Cc2ccccc2-c2c(nnn2*)-c2ccccc21 ; *N1Cc2ccccc2-c2nnn(*)c2-c2ccccc21]

In some examples, the reactive group reacts with an azide on a modified antibody or antigen binding fragment thereof. In some examples, the reactive group is an alkyne, e.g.,

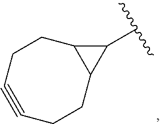 [SMILES: *C1C2CCC#CCCC12]

which can react via click chemistry with an azide, e.g.,

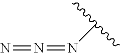

to form a click chemistry product, e.g.,

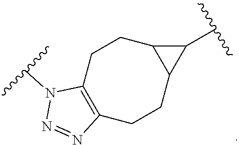 [SMILES: *C1C2CCc3nnn(*)c3CCC12]

In some examples, the reactive group is an alkyne, e.g.,

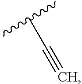 [SMILES: *C#C]

which can react via click chemistry with an azide, e.g.,

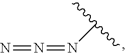

to form a click chemistry product, e.g.,

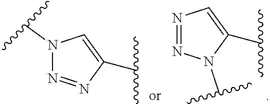

In some examples, the reactive group is a functional group, e.g.,

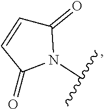 [SMILES: *N1C(=O)C=CC1=O]

which reacts with a cysteine residue on an antibody or antigen-binding fragment thereof, to form a carbon-sulfur bond thereto, e.g.,

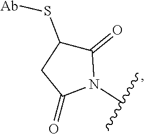

wherein Ab refers to an antibody or antigen-binding fragment thereof and S refers to the sulfur (S) atom on a cysteine residue through which the functional group bonds to the Ab. In some examples, the reactive group is a functional group, e.g.,

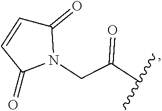 [SMILES: *C(=O)CN1C(=O)C=CC1=O]

which reacts with a lysine residue on an antibody or antigen-binding fragment thereof, to form an amide bond thereto, e.g.,

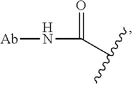

wherein Ab refers to an antibody or antigen-binding fragment thereof and —NH— refers to the —NH-atoms on a lysine side chain residue through which the functional group bonds to the Ab.

As used herein, the phrase “biodegradable moiety” refers to a moiety that degrades in vivo to non-toxic, biocompatible components which can be cleared from the body by ordinary biological processes. In some embodiments, a biodegradable moiety substantially or completely degrades in vivo over the course of about 90 days or less, about 60 days or less, or about 30 days or less, where the extent of degradation is based on percent mass loss of the biodegradable moiety, and wherein complete degradation corresponds to 100% mass loss. Exemplary biodegradable moieties include, without limitation, aliphatic polyesters such as poly(s-caprolactone) (PCL), poly(3-hydroxybutyrate) (PHB), poly(glycolic acid) (PGA), poly(lactic acid) (PLA) and its copolymers with glycolic acid (i.e., poly(D,L-lactide-coglycolide) (PLGA) (Vert M, Schwach G, Engel R and Coudane J (1998) J Control Release 53(1-3):85-92; Jain R A (2000) Biomaterials 21(23):2475-2490; Uhrich K E, Cannizzaro S M, Langer R S and Shakesheff K M (1999) Chemical Reviews 99(11): 3181-3198; and Park T G (1995) Biomaterials 16(15):1123-1130, each of which are incorporated herein by reference in their entirety).

As used herein, the phrase “binding agent linker,” or “BL” refers to any divalent, trivalent, or multi-valent group or moiety that links, connects, or bonds a binding agent (e.g., an antibody or an antigen-binding fragment thereof) with a payload compound set forth herein (e.g., tubulysins) and, optionally, with one or more side chain compounds. Generally, suitable binding agent linkers for the antibody conjugates described herein are those that are sufficiently stable to exploit the circulating half-life of the antibody conjugates and, at the same time, capable of releasing its payload after antigen-mediated internalization of the conjugate. Linkers can be cleavable or non-cleavable. Cleavable linkers are linkers that are cleaved by intracellular metabolism following internalization, e.g., cleavage via hydrolysis, reduction, or enzymatic reaction. Non-cleavable linkers are linkers that release an attached payload via lysosomal degradation of the antibody following internalization. Suitable linkers include, but are not limited to, acid-labile linkers, hydrolytically-labile linkers, enzymatically cleavable linkers, reduction labile linkers, self-immolative linkers, and non-cleavable linkers. Suitable linkers also include, but are not limited to, those that are or comprise peptides, glucuronides, succinimide-thioethers, polyethylene glycol (PEG) units, hydrazones, mal-caproyl units, dipeptide units, valine-citruline units, and para-aminobenzyloxycarbonyl (PABC), para-aminobenzyl (PAB) units. In some embodiments, the binding agent linker (BL) includes a moiety that is formed by the reaction of the reactive group (RG) of a reactive linker (RL) and reactive portion of the binding agent, e.g., antibody, modified antibody, or antigen binding fragment thereof.

In some examples, the BL includes the following moiety

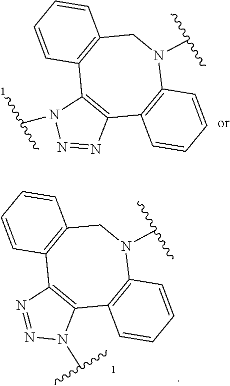

wherein [symbol] is the bond to the binding agent. In some examples, the BL includes the following moiety

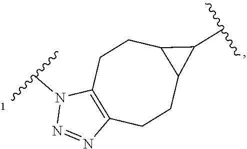

wherein [symbol] is the bond to the binding agent. In some examples, the BL includes the following moiety

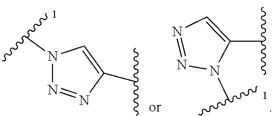

wherein [symbol] is the bond to the binding agent. In some examples, the BL includes the following moiety

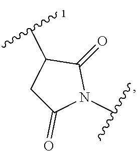

wherein [symbol] is the bond to the cysteine of the antibody or antigen-binding fragment thereof. In some examples, the BL includes the following moiety:

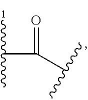

wherein [symbol] is the bond to the lysine of the antibody or antigen-binding fragment thereof.

As applied to polypeptides, the phrase “substantial similarity” or “substantially similar” means that two peptide sequences, when optimally aligned, such as by the programs GAP or BESTFIT using default gap weights, share at least 95% sequence identity, or at least 98% or 99% sequence identity. Sequence similarity may also be determined using the BLAST algorithm, described in Altschul et al. J. Mol. Biol. 215: 403-10 (using the published default settings), or available at blast.ncbi.nlm.nih.gov/Blast.cgi. In certain embodiments, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Methods for making this adjustment are well-known to those of skill in the art. See, e.g., Pearson (1994) Methods Mol. Biol. 24: 307-331. Examples of groups of amino acids that have side chains with similar chemical properties include (1) aliphatic side chains: glycine, alanine, valine, leucine, and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate; and (7) sulfur-containing side chains are cysteine and methionine. Particularly useful conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

As used herein, “enantiomeric excess (ee)” refers to a dimensionless mol ratio describing the purity of chiral substances that contain, for example, a single stereogenic center. For instance, an enantiomeric excess of zero would indicate a racemic (e.g., 50:50 mixture of enantiomers, or no excess of one enantiomer over the other). By way of further example, an enantiomeric excess of ninety-nine would indicate a nearly stereopure enantiomeric compound (i.e., large excess of one enantiomer over the other). The percentage enantiomeric excess, % ee=([(R)-compound]-[(S)-compound])/([(R)-compound]+[(S)-compound])×100, where the (R)-compound>(S)-compound; or % ee=([(S)-compound]-[(R)-compound])/([(S)-compound]++[(R)-compound])×100, where the (S)-compound>(R)-compound. Moreover, as used herein, “diastereomeric excess (de)” refers to a dimensionless mol ratio describing the purity of chiral substances that contain more than one stereogenic center. For example, a diastereomeric excess of zero would indicate an equimolar mixture of diastereoisomers. By way of further example, diastereomeric excess of ninety-nine would indicate a nearly stereopure diastereomeric compound (i.e., large excess of one diastereomer over the other). Diastereomeric excess may be calculated via a similar method to ee. As would be appreciated by a person of skill, de is usually reported as percent de (% de). % de may be calculated in a similar manner to % ee.

In certain embodiments, certain compounds or payloads listed in Table P below are excluded from the subject matter described herein.

In certain embodiments, compounds provided herein include any or all of compounds IVa, IVa′, IVb, IVc, IVd, IVe, IVf, IVg, IVh, IVj, IVk, IVl, IVm, IVn, IVo, IVp, IVq, IVr, IVs, IVt, IVu, IVvA, IVvB, IVw, IVx, IVy, Va, Va′, Vb, Vc, Vd, Ve, Vf, Vg, Vh, Vi, Vj, Vk, VIa, IVb, VIc, VId, VIe, VIf, VIg, VIh, Vi, VIi, VII, VIII, IX, X, D-5a, and D-5c in Table P. In certain embodiments, compounds provided herein exclude any or all of compounds IVa, IVa′, IVb, IVc, IVd, IVe, IVf, IVg, IVh, IVj, IVk, IVl, IVm, IVn, IVo, IVp, IVq, IVr, IVs, IVt, IVu, IVvA, IVvB, IVw, IVx, IVy, Va, Va′, Vb, Vc, Vd, Ve, Vf, Vg, Vh, Vi, Vj, Vk, VIa, IVb, VIc, VId, VIe, VIf, VIg, VIh, Vi, VIi, VII, VIII, IX, X, D-5a, and D-5c in Table P. For example, in certain embodiments, compounds provided herein include residues of any or all of compounds IVa, IVa′, IVb, IVc, IVd, IVe, IVf, IVg, IVh, IVj, IVk, IVl, IVm, IVn, IVo, IVp, IVq, IVr, IVs, IVt, IVu, IVvA, IVvB, IVw, IVx, IVy, Va, Va′, Vb, Vc, Vd, Ve, Vf, Vg, Vh, Vi, Vj, Vk, VIa, IVb, VIc, VId, VIe, VIf, VIg, VIh, Vi, VIi, VII, VIII, IX, X, D-5a, and D-5c linked to linkers and/or binding agents described herein. In certain embodiments, compounds provided herein exclude residues of any or all of compounds IVa, IVa′, IVb, IVc, IVd, IVe, IVf, IVg, IVh, IVj, IVk, IVl, IVm, IVn, IVo, IVp, IVq, IVr, IVs, IVt, IVu, IVvA, IVvB, IVw, IVx, IVy, Va, Va′, Vb, Vc, Vd, Ve, Vf, Vg, Vh, Vi, Vj, Vk, VIa, IVb, VIc, VId, VIe, VIf, VIg, VIh, Vi, VIi, VII, VIII, IX, X, D-5a, and D-5c linked to linkers and/or binding agents described herein.

TABLE P

| Compound | Structure |
|---|---|
| IVa | 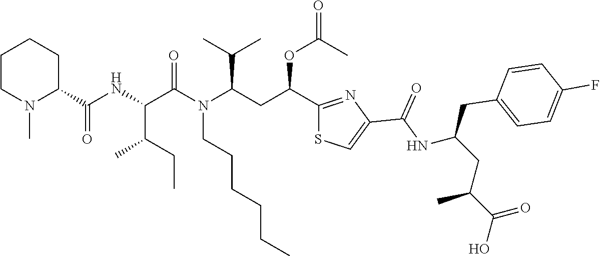 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)C[C@H](C)C(=O)O)cs1)C(C)C] |
| IVa′ | 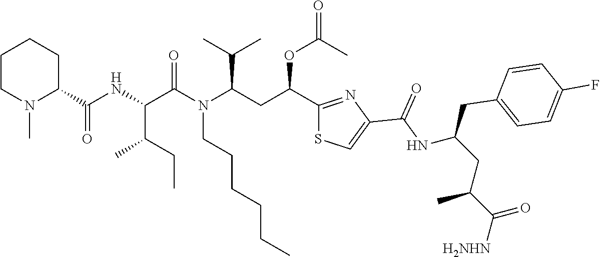 |
| IVb | 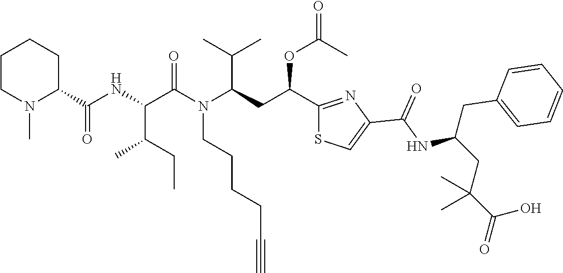 [SMILES: C#CCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccccc2)CC(C)(C)C(=O)O)cs1)C(C)C] |
| IVc | 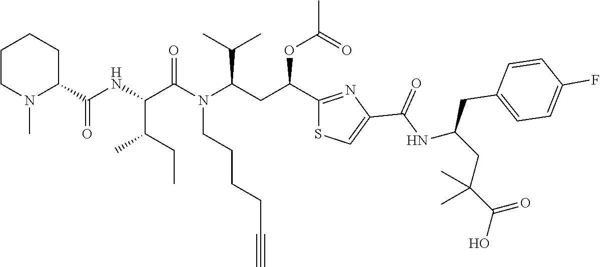 [SMILES: C#CCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)O)cs1)C(C)C] |
| IVd | 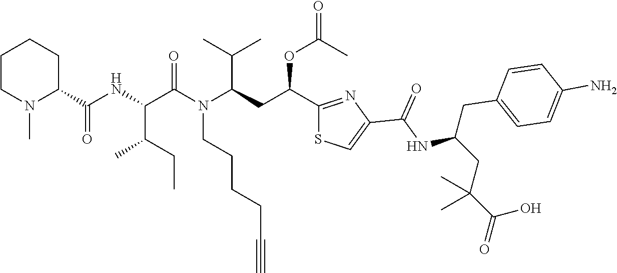 [SMILES: C#CCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IVe | 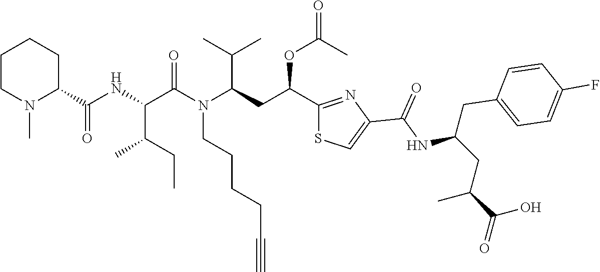 [SMILES: C#CCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)C[C@H](C)C(=O)O)cs1)C(C)C] |
| IVf | 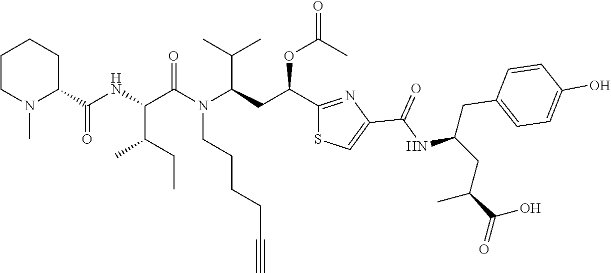 [SMILES: C#CCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)C[C@H](C)C(=O)O)cs1)C(C)C] |
| IVg | 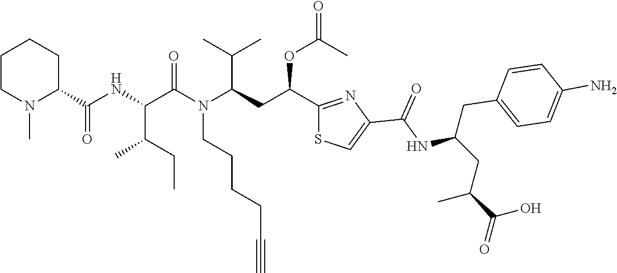 [SMILES: C#CCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)C[C@H](C)C(=O)O)cs1)C(C)C] |
| IVh | 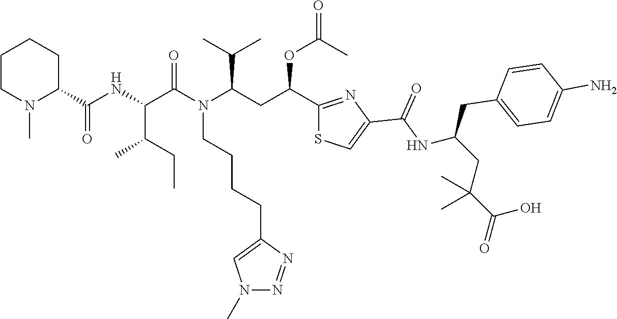 [SMILES: CC[C@H](C)[C@H](NC(=O)[C@H]1CCCCN1C)C(=O)N(CCCCc1cn(C)nn1)[C@H](C[C@@H](OC(C)=O)c1nc(C(=O)N[C@@H](Cc2ccc(N)cc2)CC(C)(C)C(=O)O)cs1)C(C)C] |
| IVj | 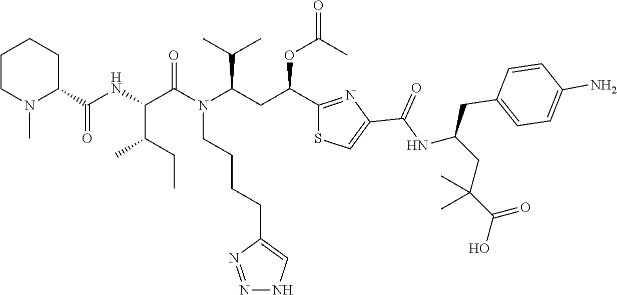 [SMILES: CC[C@H](C)[C@H](NC(=O)[C@H]1CCCCN1C)C(=O)N(CCCCc1c[nH]nn1)[C@H](C[C@@H](OC(C)=O)c1nc(C(=O)N[C@@H](Cc2ccc(N)cc2)CC(C)(C)C(=O)O)cs1)C(C)C] |
| IVk | 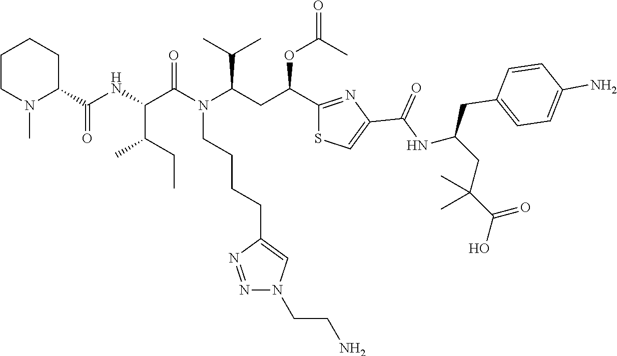 [SMILES: CC[C@H](C)[C@H](NC(=O)[C@H]1CCCCN1C)C(=O)N(CCCCc1cn(CCN)nn1)[C@H](C[C@@H](OC(C)=O)c1nc(C(=O)N[C@@H](Cc2ccc(N)cc2)CC(C)(C)C(=O)O)cs1)C(C)C] |
| IVl | 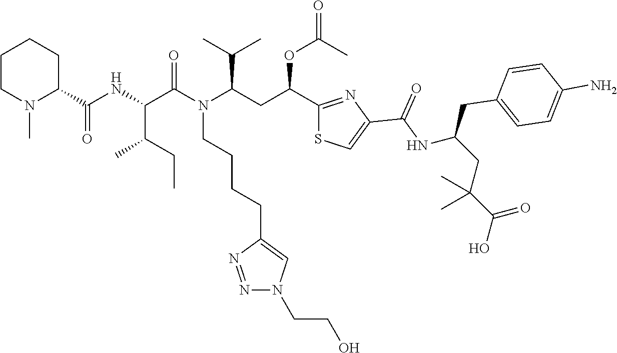 [SMILES: CC[C@H](C)[C@H](NC(=O)[C@H]1CCCCN1C)C(=O)N(CCCCc1cn(CCO)nn1)[C@H](C[C@@H](OC(C)=O)c1nc(C(=O)N[C@@H](Cc2ccc(N)cc2)CC(C)(C)C(=O)O)cs1)C(C)C] |
| IVm | 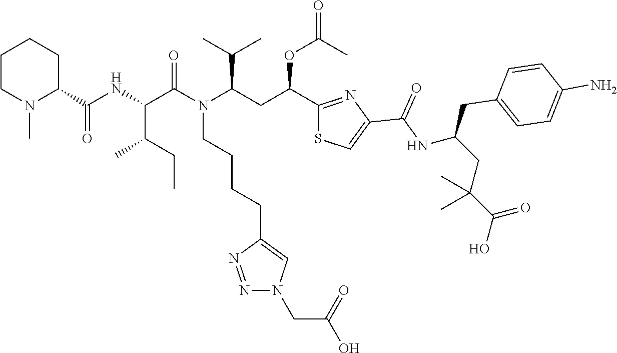 [SMILES: CC[C@H](C)[C@H](NC(=O)[C@H]1CCCCN1C)C(=O)N(CCCCc1cn(CC(=O)O)nn1)[C@H](C[C@@H](OC(C)=O)c1nc(C(=O)N[C@@H](Cc2ccc(N)cc2)CC(C)(C)C(=O)O)cs1)C(C)C] |
| IVn | 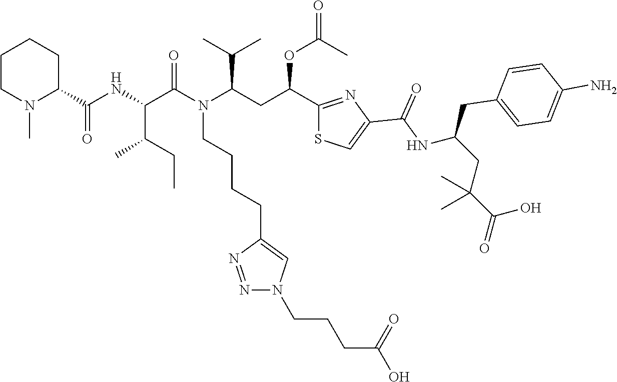 [SMILES: CC[C@H](C)[C@H](NC(=O)[C@H]1CCCCN1C)C(=O)N(CCCCc1cn(CCCC(=O)O)nn1)[C@H](C[C@@H](OC(C)=O)c1nc(C(=O)N[C@@H](Cc2ccc(N)cc2)CC(C)(C)C(=O)O)cs1)C(C)C] |
| IVo | 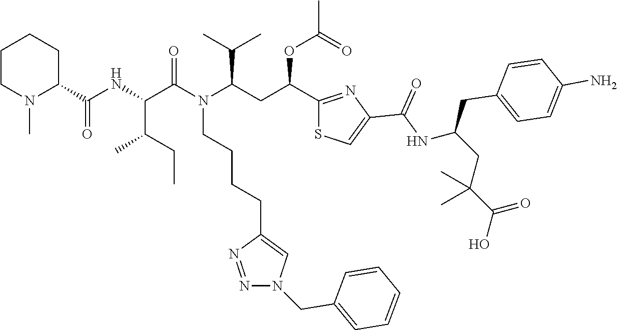 [SMILES: CC[C@H](C)[C@H](NC(=O)[C@H]1CCCCN1C)C(=O)N(CCCCc1cn(Cc2ccccc2)nn1)[C@H](C[C@@H](OC(C)=O)c1nc(C(=O)N[C@@H](Cc2ccc(N)cc2)CC(C)(C)C(=O)O)cs1)C(C)C] |
| IVp | 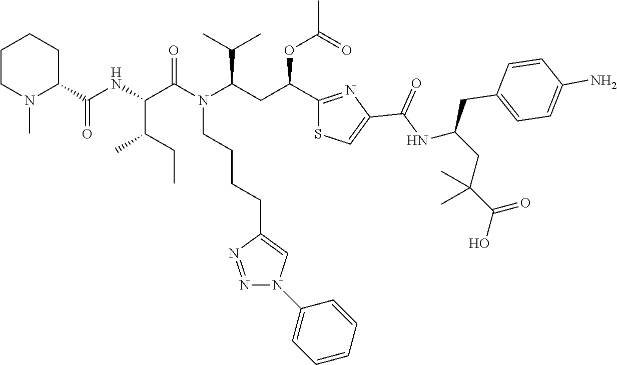 [SMILES: CC[C@H](C)[C@H](NC(=O)[C@H]1CCCCN1C)C(=O)N(CCCCc1cn(-c2ccccc2)nn1)[C@H](C[C@@H](OC(C)=O)c1nc(C(=O)N[C@@H](Cc2ccc(N)cc2)CC(C)(C)C(=O)O)cs1)C(C)C] |
| IVq | 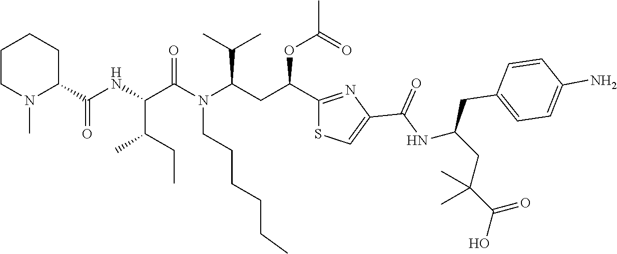 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IVr | 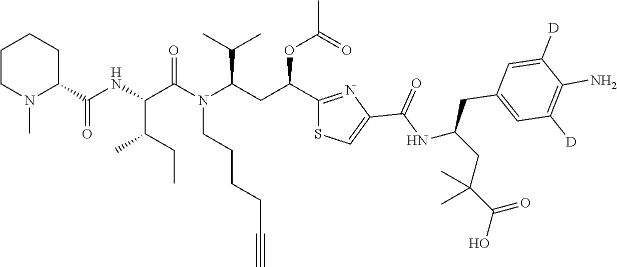 [SMILES: [2H]c1cc(C[C@@H](CC(C)(C)C(=O)O)NC(=O)c2csc([C@@H](C[C@H](C(C)C)N(CCCCC#C)C(=O)[C@@H](NC(=O)[C@H]3CCCCN3C)[C@@H](C)CC)OC(C)=O)n2)cc([2H])c1N] |
| IVs | 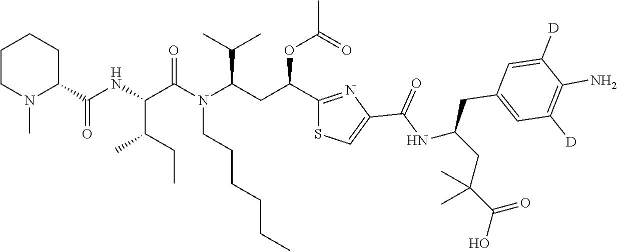 [SMILES: [2H]c1cc(C[C@@H](CC(C)(C)C(=O)O)NC(=O)c2csc([C@@H](C[C@H](C(C)C)N(CCCCCC)C(=O)[C@@H](NC(=O)[C@H]3CCCCN3C)[C@@H](C)CC)OC(C)=O)n2)cc([2H])c1N] |
| IVt | 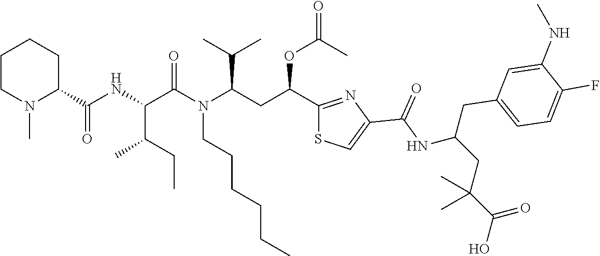 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(F)c(NC)c2)CC(C)(C)C(=O)O)cs1)C(C)C] |
| IVu | 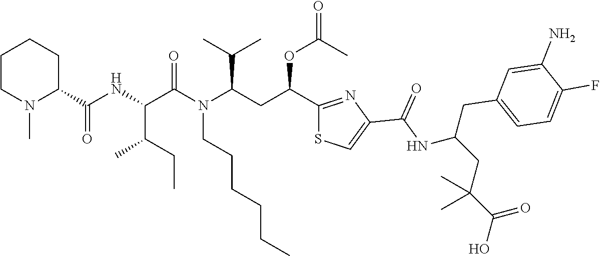 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(F)c(N)c2)CC(C)(C)C(=O)O)cs1)C(C)C] |
| IVvA | 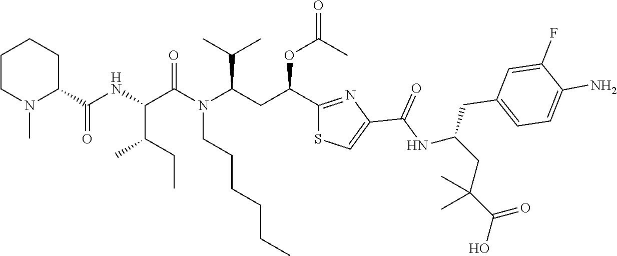 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@H](Cc2ccc(N)c(F)c2)CC(C)(C)C(=O)O)cs1)C(C)C] |
| IVvB | 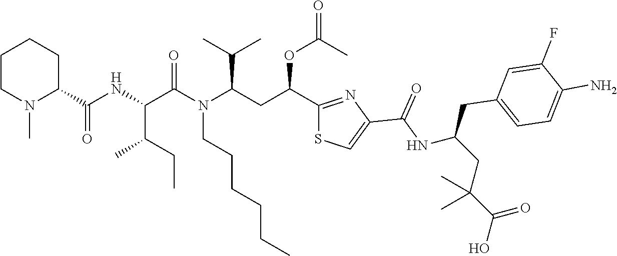 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| IVw | 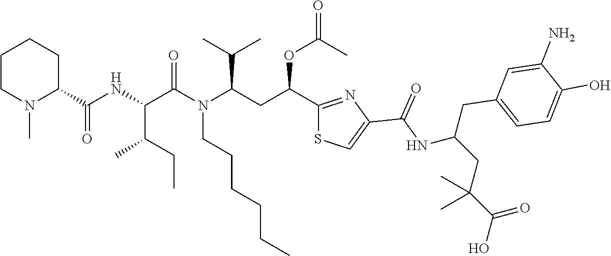 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(O)c(N)c2)CC(C)(C)C(=O)O)cs1)C(C)C] |
| IVx | 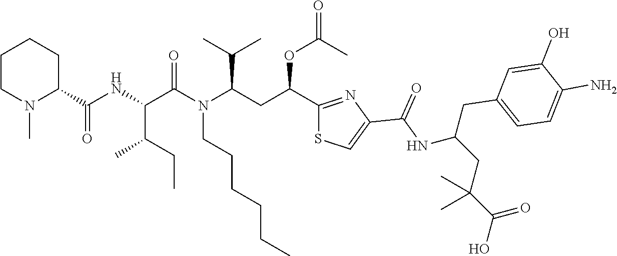 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(N)c(O)c2)CC(C)(C)C(=O)O)cs1)C(C)C] |
| IVy | 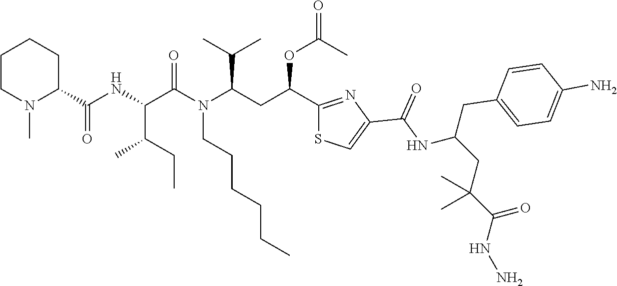 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(N)cc2)CC(C)(C)C(=O)NN)cs1)C(C)C] |
| Va | 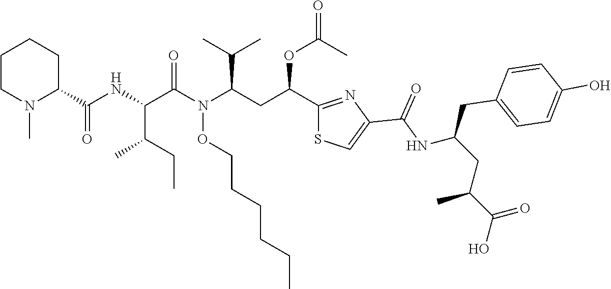 [SMILES: CCCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)C[C@H](C)C(=O)O)cs1)C(C)C] |
| Va′ | 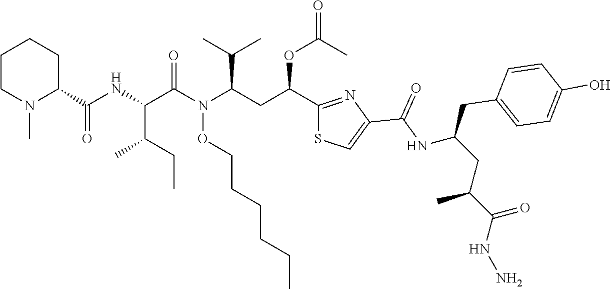 [SMILES: CCCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)C[C@H](C)C(=O)NN)cs1)C(C)C] |
| Vb | 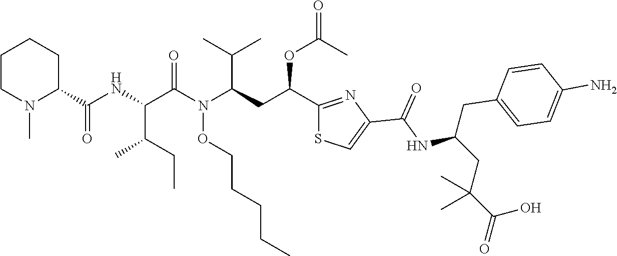 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| Vc | 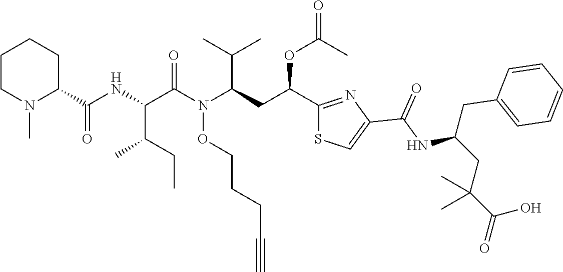 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccccc2)CC(C)(C)C(=O)O)cs1)C(C)C] |
| Vd | 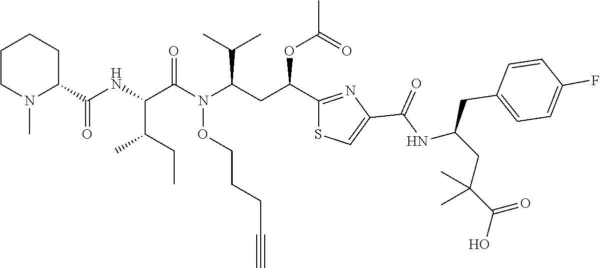 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)O)cs1)C(C)C] |
| Ve | 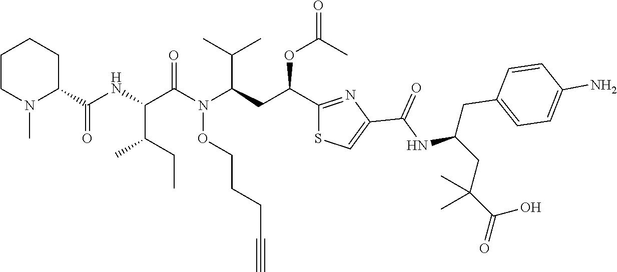 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| Vf | 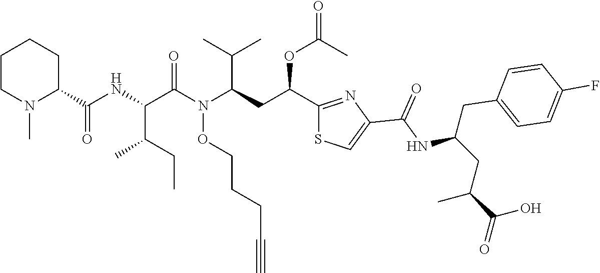 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)C[C@H](C)C(=O)O)cs1)C(C)C] |
| Vg | 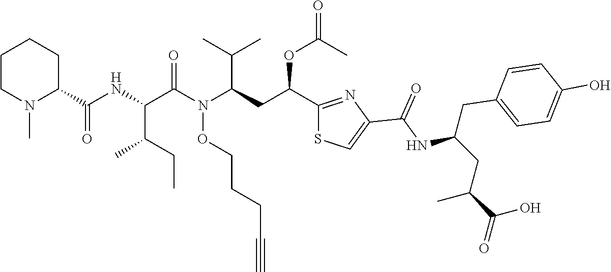 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)C[C@H](C)C(=O)O)cs1)C(C)C] |
| Vh | 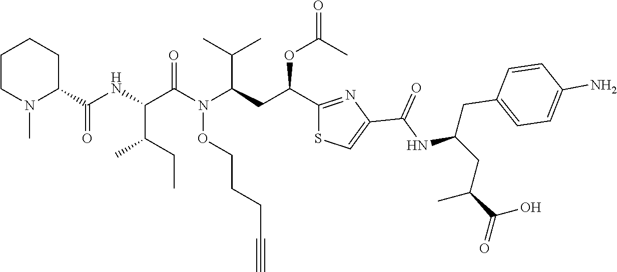 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)C[C@H](C)C(=O)O)cs1)C(C)C] |
| Vi | 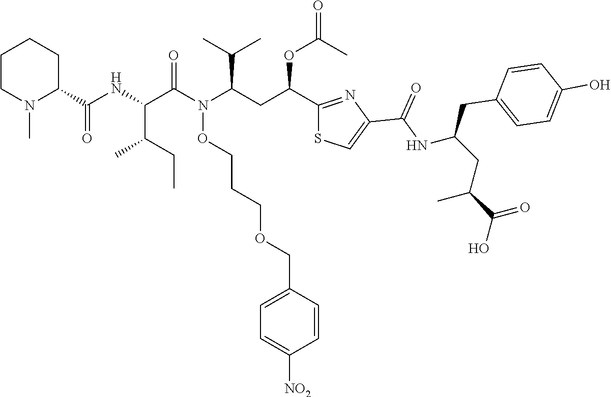 [SMILES: CC[C@H](C)[C@H](NC(=O)[C@H]1CCCCN1C)C(=O)N(OCCCOCc1ccc([N+](=O)[O-])cc1)[C@H](C[C@@H](OC(C)=O)c1nc(C(=O)N[C@@H](Cc2ccc(O)cc2)C[C@H](C)C(=O)O)cs1)C(C)C] |
| Vj | 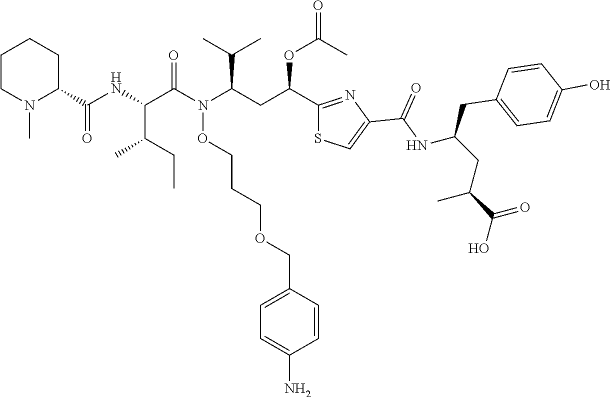 [SMILES: CC[C@H](C)[C@H](NC(=O)[C@H]1CCCCN1C)C(=O)N(OCCCOCc1ccc(N)cc1)[C@H](C[C@@H](OC(C)=O)c1nc(C(=O)N[C@@H](Cc2ccc(O)cc2)C[C@H](C)C(=O)O)cs1)C(C)C] |
| Vk | 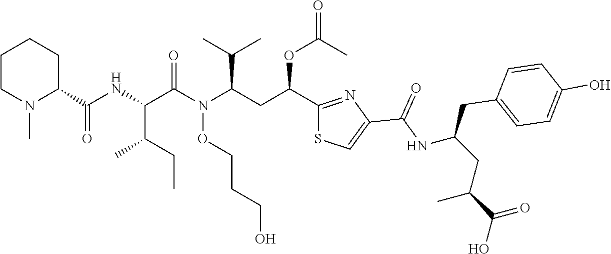 [SMILES: CC[C@H](C)[C@H](NC(=O)[C@H]1CCCCN1C)C(=O)N(OCCCO)[C@H](C[C@@H](OC(C)=O)c1nc(C(=O)N[C@@H](Cc2ccc(O)cc2)C[C@H](C)C(=O)O)cs1)C(C)C] |
| VIa | 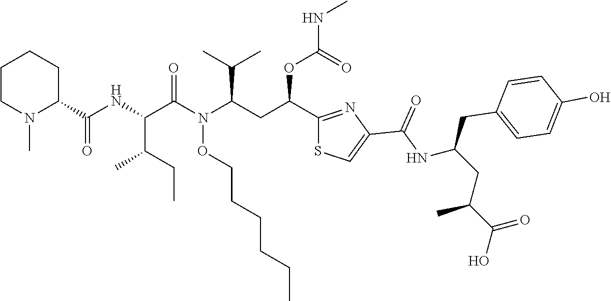 [SMILES: CCCCCCON(C(=O)[C@@H](NC(=O)[C@H]1CCCCN1C)[C@@H](C)CC)[C@H](C[C@@H](OC(=O)NC)c1nc(C(=O)N[C@@H](Cc2ccc(O)cc2)C[C@H](C)C(=O)O)cs1)C(C)C] |
| IVb | 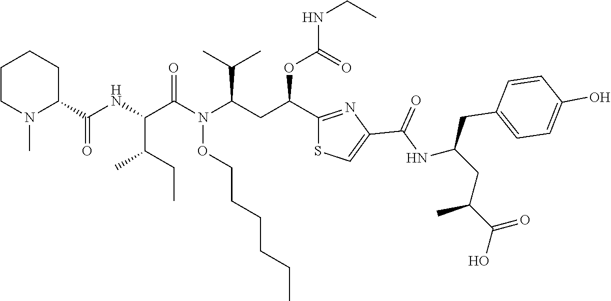 [SMILES: CCCCCCON(C(=O)[C@@H](NC(=O)[C@H]1CCCCN1C)[C@@H](C)CC)[C@H](C[C@@H](OC(=O)NCC)c1nc(C(=O)N[C@@H](Cc2ccc(O)cc2)C[C@H](C)C(=O)O)cs1)C(C)C] |
| VIc | 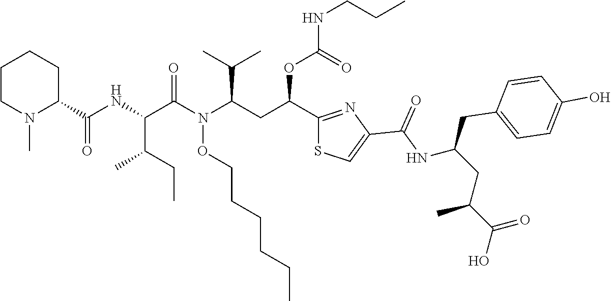 [SMILES: CCCCCCON(C(=O)[C@@H](NC(=O)[C@H]1CCCCN1C)[C@@H](C)CC)[C@H](C[C@@H](OC(=O)NCCC)c1nc(C(=O)N[C@@H](Cc2ccc(O)cc2)C[C@H](C)C(=O)O)cs1)C(C)C] |
| VId | 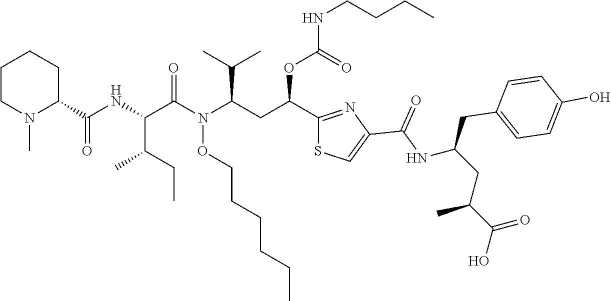 [SMILES: CCCCCCON(C(=O)[C@@H](NC(=O)[C@H]1CCCCN1C)[C@@H](C)CC)[C@H](C[C@@H](OC(=O)NCCCC)c1nc(C(=O)N[C@@H](Cc2ccc(O)cc2)C[C@H](C)C(=O)O)cs1)C(C)C] |
| VIe | 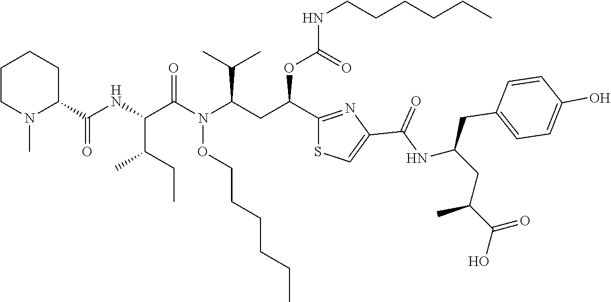 [SMILES: CCCCCCNC(=O)O[C@H](C[C@H](C(C)C)N(OCCCCCC)C(=O)[C@@H](NC(=O)[C@H]1CCCCN1C)[C@@H](C)CC)c1nc(C(=O)N[C@@H](Cc2ccc(O)cc2)C[C@H](C)C(=O)O)cs1] |
| VIf | 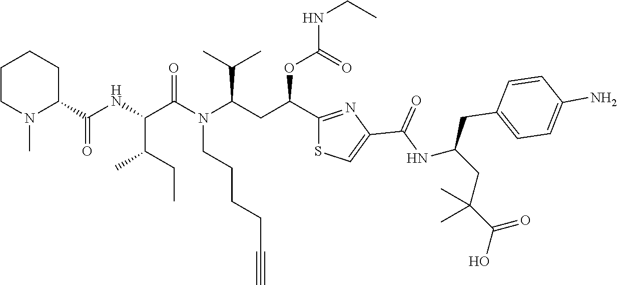 [SMILES: C#CCCCCN(C(=O)[C@@H](NC(=O)[C@H]1CCCCN1C)[C@@H](C)CC)[C@H](C[C@@H](OC(=O)NCC)c1nc(C(=O)N[C@@H](Cc2ccc(N)cc2)CC(C)(C)C(=O)O)cs1)C(C)C] |
| VIg | 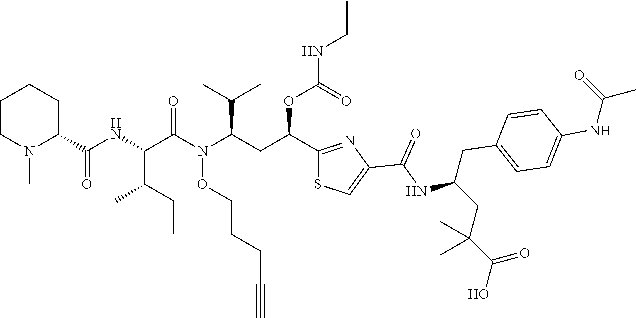 [SMILES: C#CCCCON(C(=O)[C@@H](NC(=O)[C@H]1CCCCN1C)[C@@H](C)CC)[C@H](C[C@@H](OC(=O)NCC)c1nc(C(=O)N[C@@H](Cc2ccc(NC(C)=O)cc2)CC(C)(C)C(=O)O)cs1)C(C)C] |
| VIh | 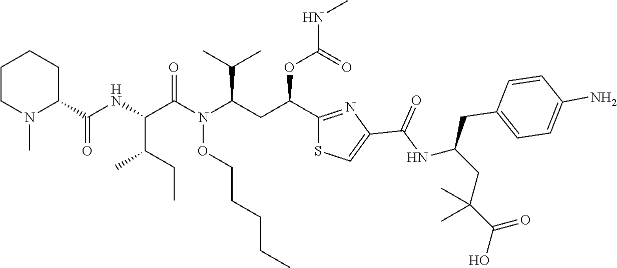 [SMILES: CCCCCON(C(=O)[C@@H](NC(=O)[C@H]1CCCCN1C)[C@@H](C)CC)[C@H](C[C@@H](OC(=O)NC)c1nc(C(=O)N[C@@H](Cc2ccc(N)cc2)CC(C)(C)C(=O)O)cs1)C(C)C] |
| Vl | 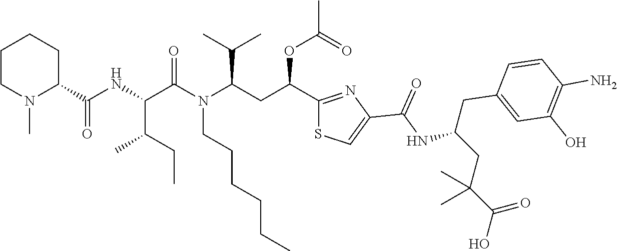 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@H](Cc2ccc(N)c(O)c2)CC(C)(C)C(=O)O)cs1)C(C)C] |
| VIi | 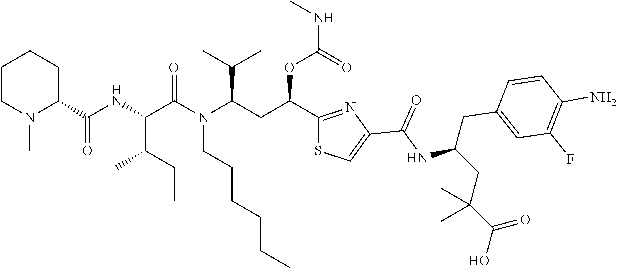 [SMILES: CCCCCCN(C(=O)[C@@H](NC(=O)[C@H]1CCCCN1C)[C@@H](C)CC)[C@H](C[C@@H](OC(=O)NC)c1nc(C(=O)N[C@@H](Cc2ccc(N)c(F)c2)CC(C)(C)C(=O)O)cs1)C(C)C] |
| VII | 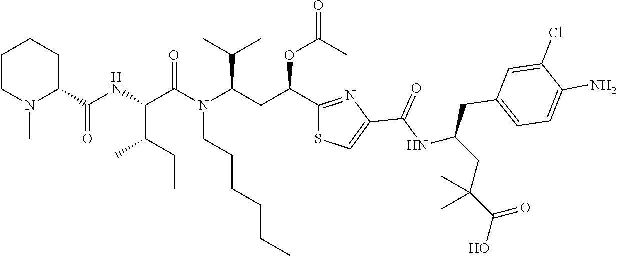 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)c(Cl)c2)CC(C)(C)C(=O)O)cs1)C(C)C] |
| VIII | 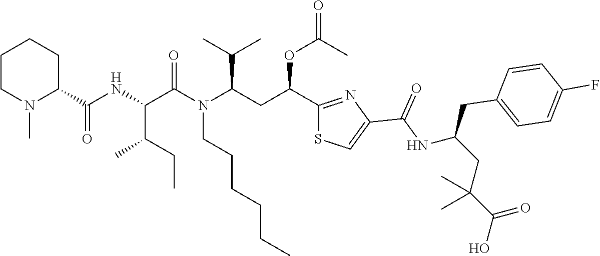 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)O)cs1)C(C)C] |
| IX | 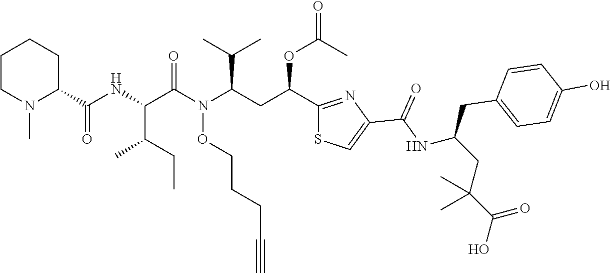 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| X | 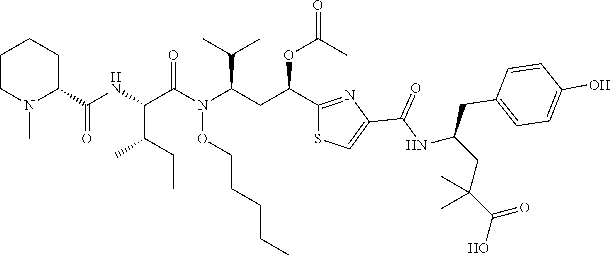 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| D-5a | 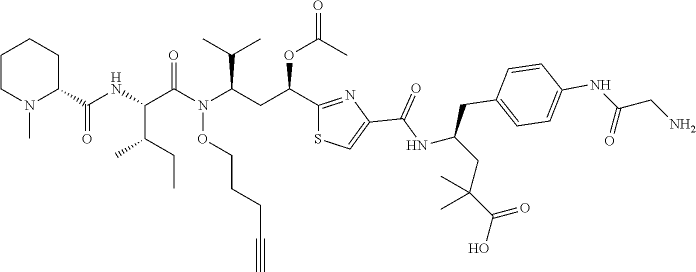 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |
| D-5c | 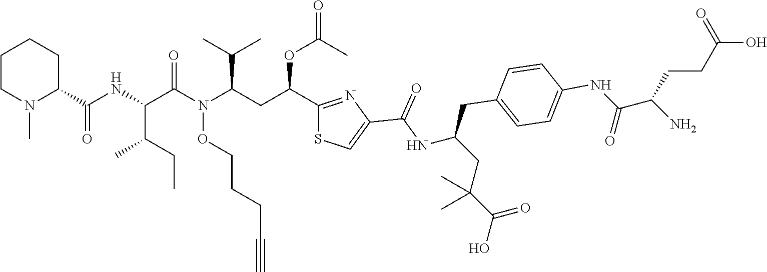 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@@H](N)CCC(=O)O)cc2)CC(C)(C)C(=O)O)cs1)C(C)C] |

In certain embodiments, certain compounds or linker-payloads listed in Table P1 below are excluded from the subject matter described herein.

In certain embodiments, the compounds provided herein include any or all of compounds LP1-IVa, LP2-Va, LP3-IVd, LP4-Ve, LP5-IVd, LP6-Vb, LP7-IVd, LP9-IVvB, LP10-VIh, LP11-IVvB, LP12-VIi, LP13-Ve, LP14-Ve, LP15-VIh, LP16-Ve, LP17-Ve, LP18-Ve, LP19-Ve, LP20-Ve, LP21-Ve, LP22-Ve, LP23-Vb, LP24-Vb, LP25-Ve, and LP26-Ve in Table P1. In certain embodiments, the compounds provided herein exclude any or all of compounds LP1-IVa, LP2-Va, LP3-IVd, LP4-Ve, LP5-IVd, LP6-Vb, LP7-IVd, LP9-IVvB, LP10-VIh, LP11-IVvB, LP12-VIi, LP13-Ve, LP14-Ve, LP15-VIh, LP16-Ve, LP17-Ve, LP18-Ve, LP19-Ve, LP20-Ve, LP21-Ve, LP22-Ve, LP23-Vb, LP24-Vb, LP25-Ve, and LP26-Vein Table P1. For example, in certain embodiments, compounds provided herein include residues of any or all of compounds LP1-IVa, LP2-Va, LP3-IVd, LP4-Ve, LP5-IVd, LP6-Vb, LP7-IVd, LP9-IVvB, LP10-VIh, LP11-IVvB, LP12-VIi, LP13-Ve, LP14-Ve, LP15-VIh, LP16-Ve, LP17-Ve, LP18-Ve, LP19-Ve, LP20-Ve, LP21-Ve, LP22-Ve, LP23-Vb, LP24-Vb, LP25-Ve, and LP26-Ve linked to binding agents described herein. In certain embodiments, compounds provided herein exclude residues of any or all of compounds LP1-IVa, LP2-Va, LP3-IVd, LP4-Ve, LP5-IVd, LP6-Vb, LP7-IVd, LP9-IVvB, LP10-VIh, LP11-IVvB, LP12-VIi, LP13-Ve, LP14-Ve, LP15-VIh, LP16-Ve, LP17-Ve, LP18-Ve, LP19-Ve, LP20-Ve, LP21-Ve, LP22-Ve, LP23-Vb, LP24-Vb, LP25-Ve, and LP26-Ve linked to binding agents described herein.

TABLE P1

| | Structures |
|---|---|
| LP1- IVa | 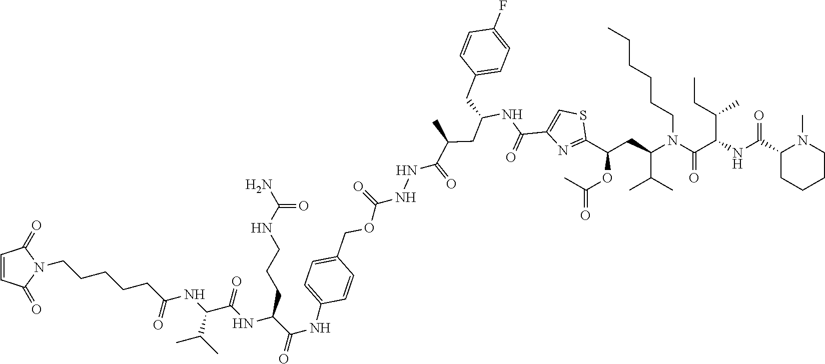 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)C[C@H](C)C(=O)NNC(=O)OCc2ccc(NC(=O)[C@H](CCCNC(N)=O)NC(=O)[C@@H](NC(=O)CCCCCN3C(=O)C=CC3=O)C(C)C)cc2)cs1)C(C)C] |
| LP2- Va | 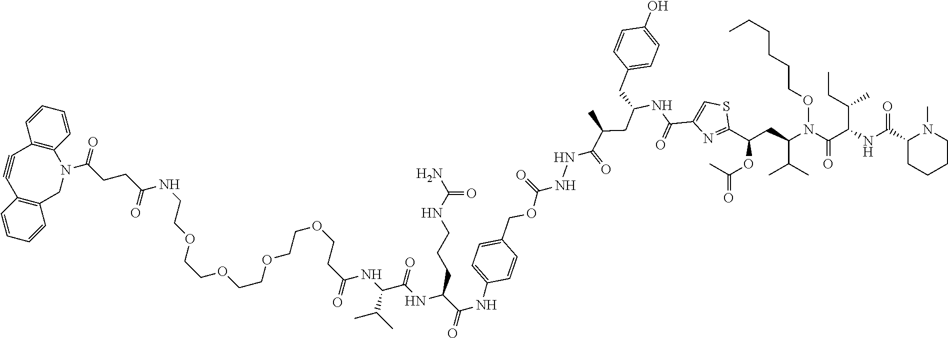 [SMILES: CCCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)C[C@H](C)C(=O)NNC(=O)OCc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)cs1)C(C)C] |
| LP3- IVd | 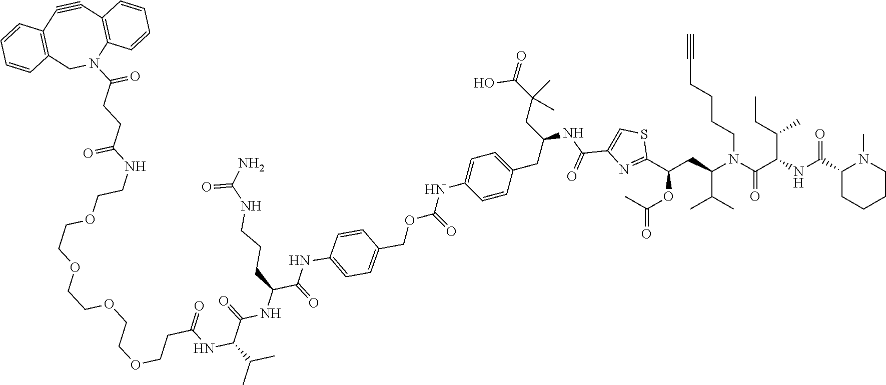 [SMILES: C#CCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)OCc3ccc(NC(=O)[C@H](CCCNC(N)=O)NC(=O)[C@@H](NC(=O)CCOCCOCCOCCOCCNC(=O)CCC(=O)N4Cc5ccccc5C#Cc5ccccc54)C(C)C)cc3)cc2)CC(C)(C)C(=O)O)cs1)C(C)C] |
| LP4- Ve | 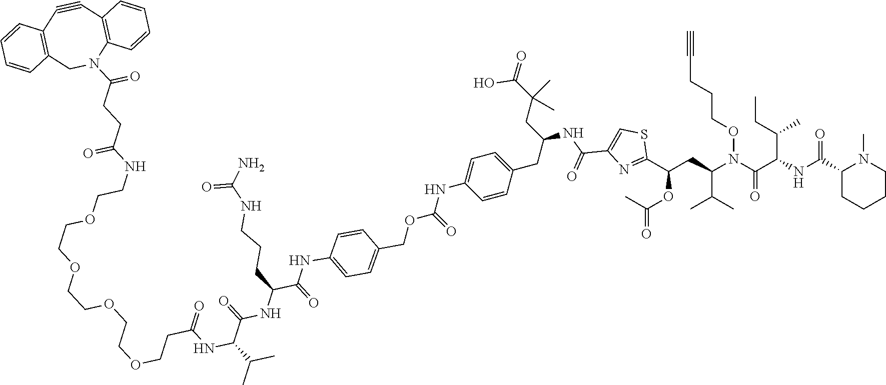 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)OCc3ccc(NC(=O)[C@H](CCCNC(N)=O)NC(=O)[C@@H](NC(=O)CCOCCOCCOCCOCCNC(=O)CCC(=O)N4Cc5ccccc5C#Cc5ccccc54)C(C)C)cc3)cc2)CC(C)(C)C(=O)O)cs1)C(C)C] |
| LP5- IVd | 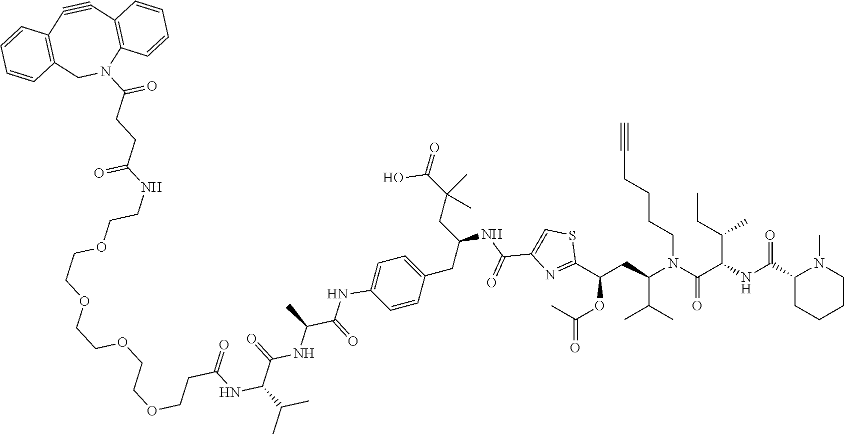 [SMILES: C#CCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](C)NC(=O)[C@@H](NC(=O)CCOCCOCCOCCOCCNC(=O)CCC(=O)N3Cc4ccccc4C#Cc4ccccc43)C(C)C)cc2)CC(C)(C)C(=O)O)cs1)C(C)C] |
| LP6- Vb | 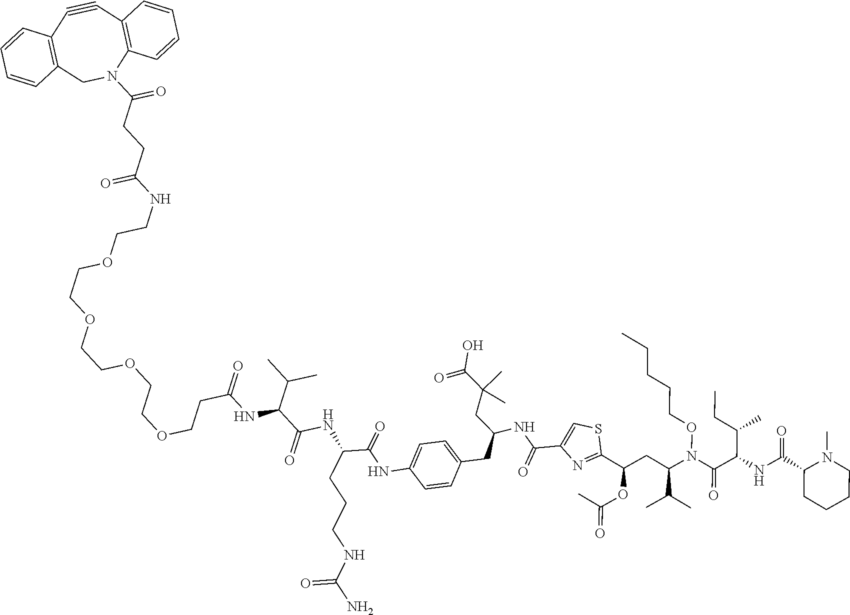 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)CC(C)(C)C(=O)O)cs1)C(C)C] |
| LP7- IVd | 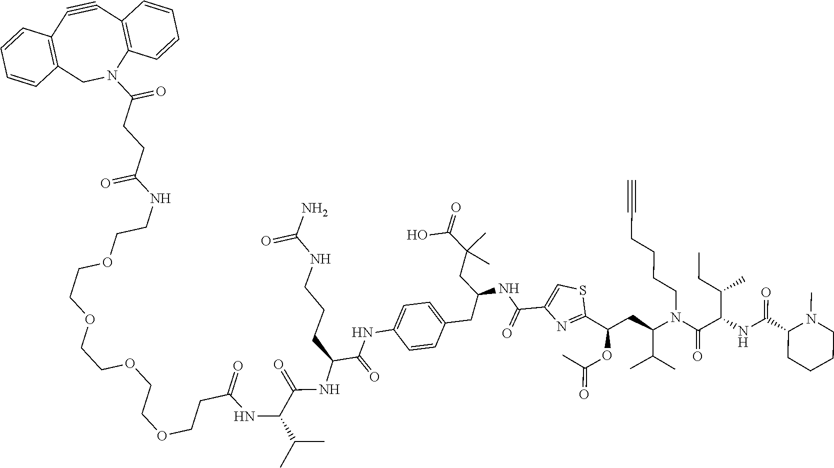 [SMILES: C#CCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)CC(C)(C)C(=O)O)cs1)C(C)C] |
| LP9- IVvB | 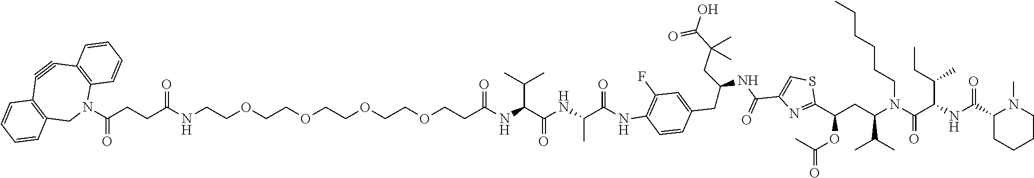 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](C)NC(=O)[C@@H](NC(=O)CCOCCOCCOCCOCCNC(=O)CCC(=O)N3Cc4ccccc4C#Cc4ccccc43)C(C)C)c(F)c2)CC(C)(C)C(=O)O)cs1)C(C)C] |
| LP10- VIh | 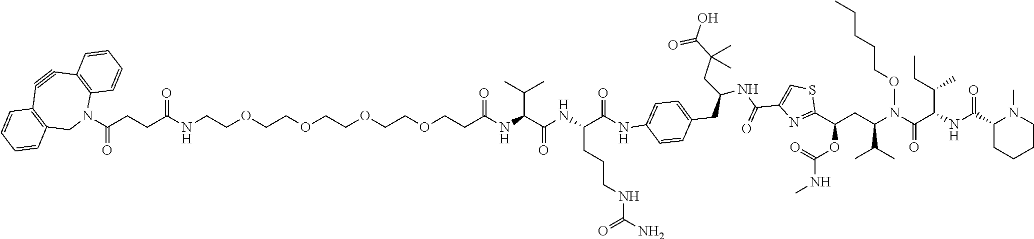 [SMILES: CCCCCON(C(=O)[C@@H](NC(=O)[C@H]1CCCCN1C)[C@@H](C)CC)[C@H](C[C@@H](OC(=O)NC)c1nc(C(=O)N[C@@H](Cc2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)CC(C)(C)C(=O)O)cs1)C(C)C] |
| LP11- IVvB | 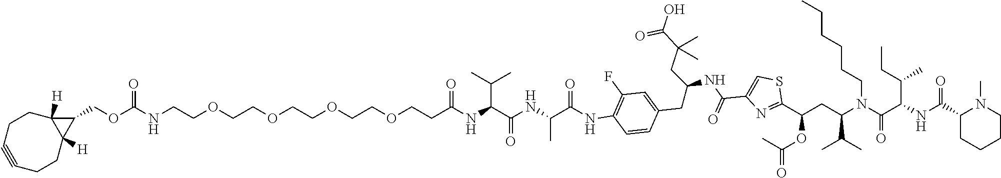 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](C)NC(=O)[C@@H](NC(=O)CCOCCOCCOCCOCCNC(=O)OC[C@@H]3[C@@H]4CCC#CCC[C@@H]43)C(C)C)c(F)c2)CC(C)(C)C(=O)O)cs1)C(C)C] |
| LP12- VIi | 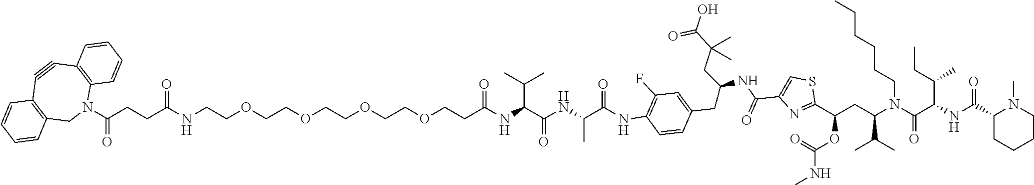 [SMILES: CCCCCCN(C(=O)[C@@H](NC(=O)[C@H]1CCCCN1C)[C@@H](C)CC)[C@H](C[C@@H](OC(=O)NC)c1nc(C(=O)N[C@@H](Cc2ccc(NC(=O)[C@H](C)NC(=O)[C@@H](NC(=O)CCOCCOCCOCCOCCNC(=O)CCC(=O)N3Cc4ccccc4C#Cc4ccccc43)C(C)C)c(F)c2)CC(C)(C)C(=O)O)cs1)C(C)C] |
| LP13- Ve | 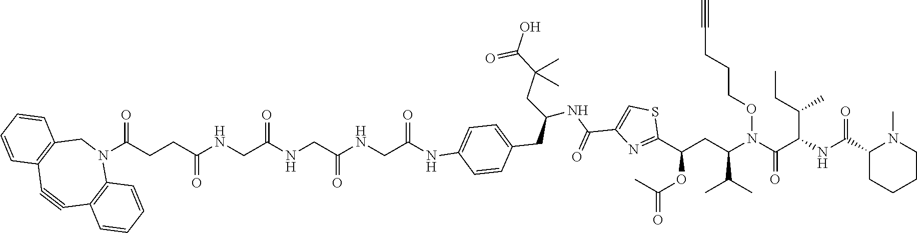 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)CNC(=O)CNC(=O)CCC(=O)N3Cc4ccccc4C#Cc4ccccc43)cc2)CC(C)(C)C(=O)O)cs1)C(C)C] |
| LP14- Ve | 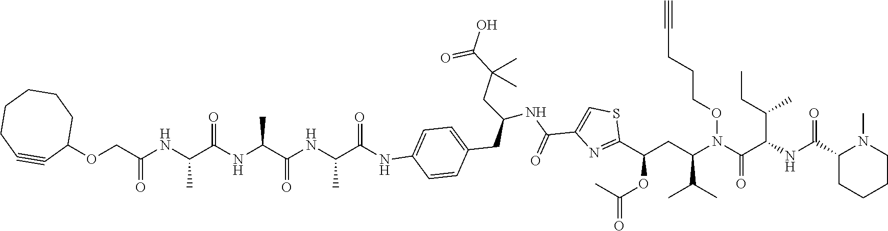 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](C)NC(=O)[C@H](C)NC(=O)[C@H](C)NC(=O)COC3C#CCCCCC3)cc2)CC(C)(C)C(=O)O)cs1)C(C)C] |
| LP15- VIh | 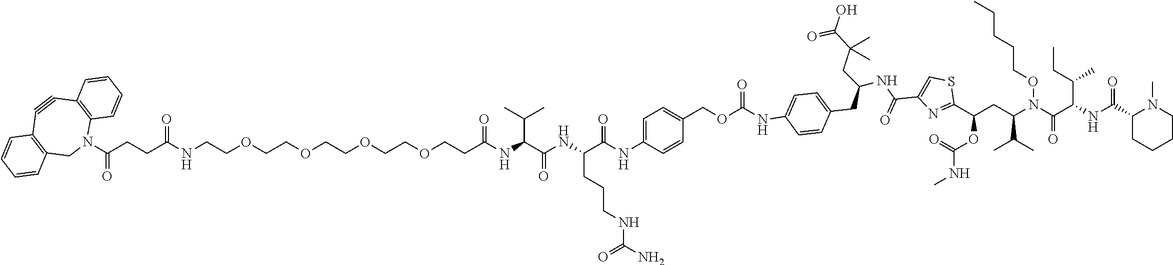 [SMILES: CCCCCON(C(=O)[C@@H](NC(=O)[C@H]1CCCCN1C)[C@@H](C)CC)[C@H](C[C@@H](OC(=O)NC)c1nc(C(=O)N[C@@H](Cc2ccc(NC(=O)OCc3ccc(NC(=O)[C@H](CCCNC(N)=O)NC(=O)[C@@H](NC(=O)CCOCCOCCOCCOCCNC(=O)CCC(=O)N4Cc5ccccc5C#Cc5ccccc54)C(C)C)cc3)cc2)CC(C)(C)C(=O)O)cs1)C(C)C] |
| LP16- Ve | 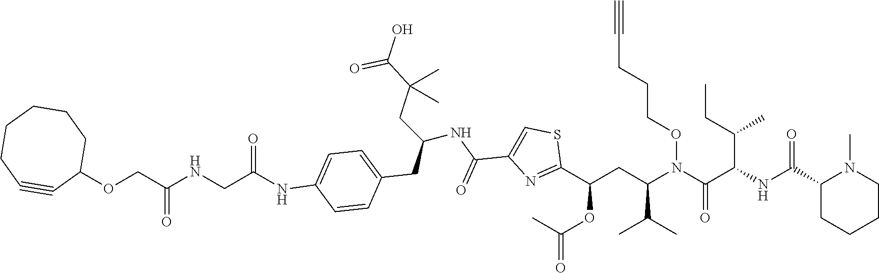 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)COC3C#CCCCCC3)cc2)CC(C)(C)C(=O)O)cs1)C(C)C] |
| LP17- Ve | 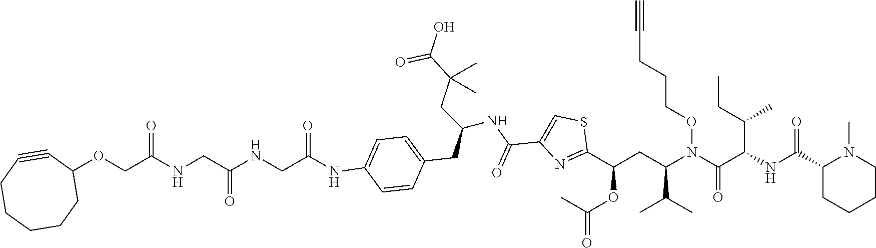 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)CNC(=O)COC3C#CCCCCC3)cc2)CC(C)(C)C(=O)O)cs1)C(C)C] |
| LP18- Ve | 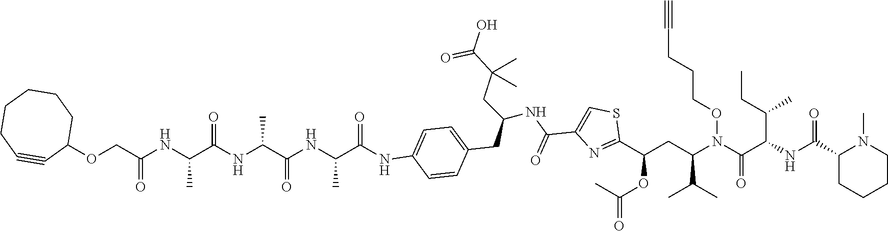 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](C)NC(=O)[C@@H](C)NC(=O)[C@H](C)NC(=O)COC3C#CCCCCC3)cc2)CC(C)(C)C(=O)O)cs1)C(C)C] |
| LP19- Ve | 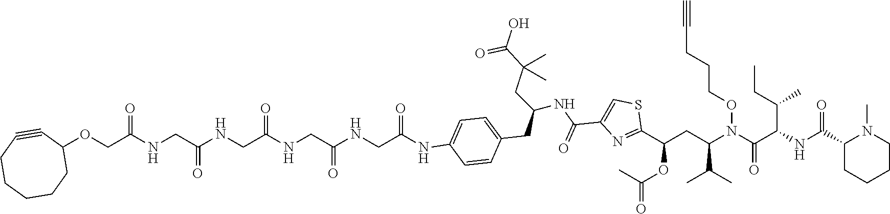 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)CNC(=O)CNC(=O)CNC(=O)COC3C#CCCCCC3)cc2)CC(C)(C)C(=O)O)cs1)C(C)C] |
| LP20- Ve | 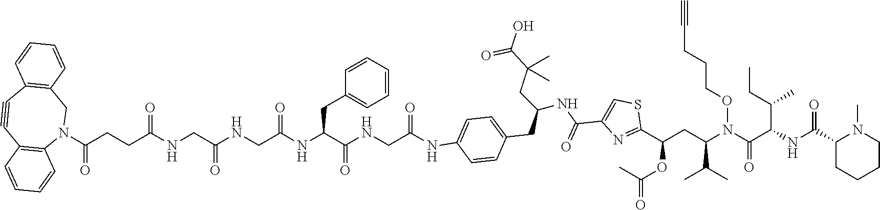 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)[C@H](Cc3ccccc3)NC(=O)CNC(=O)CNC(=O)CCC(=O)N3Cc4ccccc4C#Cc4ccccc43)cc2)CC(C)(C)C(=O)O)cs1)C(C)C] |
| LP21- Ve | 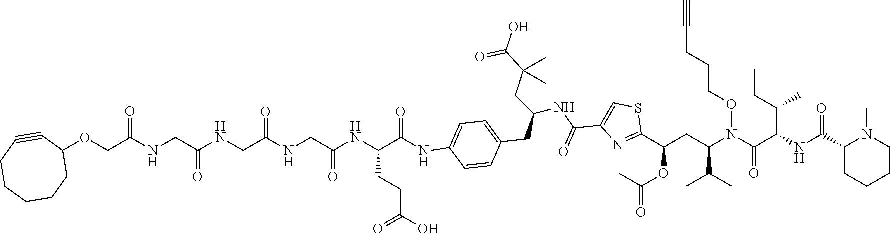 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](CCC(=O)O)NC(=O)CNC(=O)CNC(=O)CNC(=O)COC3C#CCCCCC3)cc2)CC(C)(C)C(=O)O)cs1)C(C)C] |
| LP22- Ve | 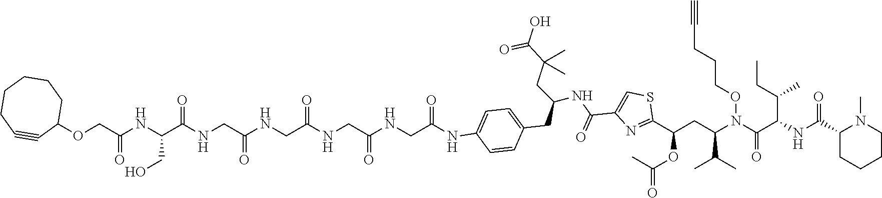 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)CNC(=O)CNC(=O)CNC(=O)[C@H](CO)NC(=O)COC3C#CCCCCC3)cc2)CC(C)(C)C(=O)O)cs1)C(C)C] |
| LP23- Vb | 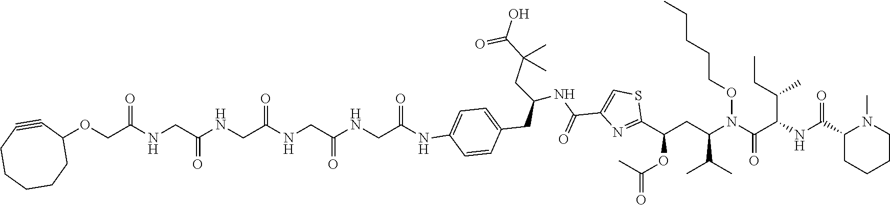 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)CNC(=O)CNC(=O)CNC(=O)COC3C#CCCCCC3)cc2)CC(C)(C)C(=O)O)cs1)C(C)C] |
| LP24- Vb | 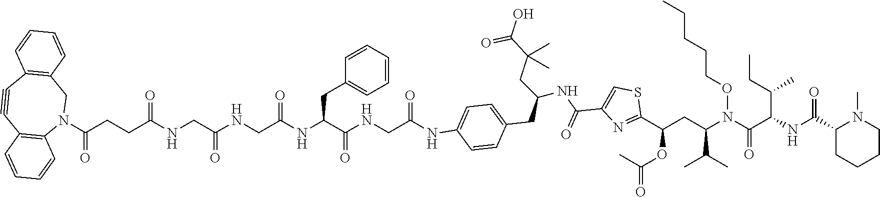 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)[C@H](Cc3ccccc3)NC(=O)CNC(=O)CNC(=O)CCC(=O)N3Cc4ccccc4C#Cc4ccccc43)cc2)CC(C)(C)C(=O)O)cs1)C(C)C] |
| LP25- Ve | 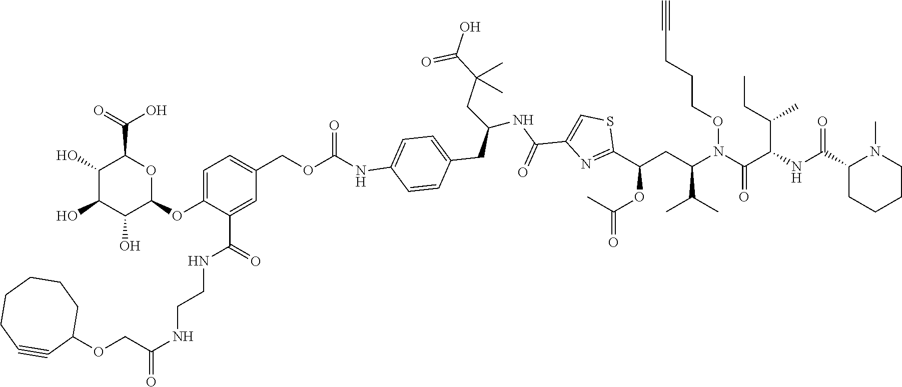 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)OCc3ccc(O[C@@H]4O[C@H](C(=O)O)[C@@H](O)[C@H](O)[C@H]4O)c(C(=O)NCCNC(=O)COC4C#CCCCCC4)c3)cc2)CC(C)(C)C(=O)O)cs1)C(C)C] |
| LP26- Ve | 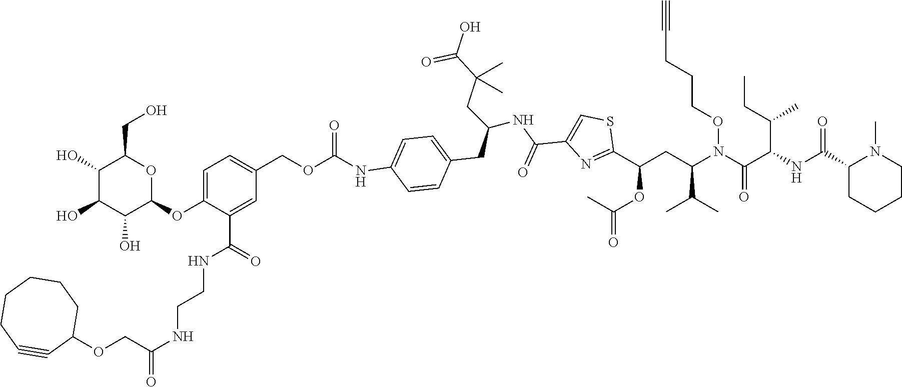 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)OCc3ccc(O[C@@H]4O[C@H](CO)[C@@H](O)[C@H](O)[C@H]4O)c(C(=O)NCCNC(=O)COC4C#CCCCCC4)c3)cc2)CC(C)(C)C(=O)O)cs1)C(C)C] |

### Compounds, Payloads, or Prodrug Payloads

Provided herein are compounds, biologically active compounds, or payloads. Without being bound by any particular theory of operation, the compounds include tubulysins and derivatives thereof, for example, prodrugs thereof. The terms or phrases “compounds,” “biologically active compounds,” “prodrugs,” “prodrug payloads,” and “payloads” are used interchangeably throughout this disclosure.

In certain embodiments, the biologically active compound (D*) or residue thereof includes, for example, amino, hydroxyl, carboxylic acid, and/or amide functionality (e.g., D*-NH₂ or D*-NH—R; D*-OH or D*-O—R; D*-COOH or D*-C(O)O—R; and/or D*-CONH₂, D*-CONH—R, or D*-NHC(O)—R). In certain embodiments herein, for example and convenience, a heterocyclic nitrogen, R², R³, R⁶, and/or R⁷ represents the amino, hydroxyl, carboxylic acid, and amide functional groups within the biologically active compounds described herein, as would be appreciated by a person of skill in the art. Alternatively stated, a person of skill would recognize that a heterocyclic nitrogen, R², R³, R⁶, and/or R⁷ may be part of the biologically active compounds described herein (e.g., D*), and may be used as a functional group for conjugation purposes. In one embodiment, the hydroxyl functionality is a primary hydroxyl moiety (e.g., D*-CH₂OH or D*-CH₂O—R; or D*-C(O)CH₂OH or D*-C(O)CH₂O—R). In another embodiment, the hydroxyl functionality is a secondary hydroxyl moiety (e.g., D*-CH(OH)R or D*-CH(O—R)R; or D*-C(O)CH(R)(OH) or D*-C(O)CH(R)(O—R)). In another embodiment, the hydroxyl functionality is a tertiary hydroxyl moiety (e.g., D*-C(R₁)(R₂)(OH) or D*-C(R₁)(R₂)(O—R); or D*-C(O)C(R₁)(R₂)(OH) or D*-C(O)C(R₁)(R₂)(O—R)). In certain embodiments, the biologically active compound (D*) or residue thereof includes amino functionality (e.g., D*-NR₂ or D*-N(R)—R). In one embodiment, the amino functionality is a primary amino moiety (e.g., D*-CH₂NR₂ or D*-CH₂N(R)—R; or D*-C(O)CH₂NR₂ or D*-C(O)CH₂N(R)—R). In another embodiment, the amino functionality is a secondary amino moiety (e.g., D*-CH(NR₂)R or D*-CH(NR—R)R; or D*-C(O)CH(R)(NR₂) or D*-C(O)CH(R)(NR—R)). In another embodiment, the amino functionality is a tertiary amino moiety (e.g., D*-C(R₁)(R₂)(NR₂) or D*-C(R₁)(R₂)(N(R)—R); or D*-C(O)C(R₁)(R₂)(NR₂) or D*-C(O)C(R₁)(R₂)(N(R)—R)). In another embodiment, the amino functionality is quaternary, as would be appreciated by a person of skill in the art. In another embodiment, the D* including the amino functionality is an aryl amine (e.g., D*-Ar—NR₂, D*-Ar—N(R)—R. Those of skill will recognize that each functional group in the previous sentences can be part of the biologically active compound D* and simultaneously be depicted in the formula for clarity, convenience, and/or emphasis. In another embodiment, the D* including the hydroxyl functionality is an aryl hydroxyl or phenolic hydroxyl (e.g., D*-Ar—OH, D*-Ar—O—R. In another embodiment, D* including the amide functionality is a tubulysin prodrug residue resulting from the reaction of a tubulysin compound or derivative, for example at R⁷ described herein, and an amino acid compound also described herein. For example, in certain embodiments, D*-NHC(O)C(S^{c})(H)NH₂ represents a tubulysin prodrug bearing an N-terminal amino acid residue, wherein S^{c} represent an amino acid side chain. By way of further example, in certain embodiments, D*-NH[C(O)C(S^{c})(H)NH]ₐₐC(O)C(S^{C})(H)NH₂ represents a tubulysin prodrug bearing an N-terminal peptide residue, wherein S^{c} represent an amino acid side chain and aa is an integer from one to one hundred. In certain embodiments, aa is one. In certain embodiments, aa is two. In certain embodiments, aa is three. In certain embodiments, aa is four. In certain embodiments, aa is five. As used herein, “amino acid side chain” refers to the additional chemical moiety on the same carbon that bears a primary or secondary amine and a carboxylic acid of an amino acid. As would be appreciated by a person of skill in the art, there are twenty-one “standard” amino acids. Exemplary “standard” amino acids include, without limitation, alanine, serine, proline, arginine, and aspartic acid. Other amino acids include, cysteine, selenocysteine, and glycine (e.g., wherein the additional chemical moiety on the same carbon that bears the primary amine and carboxylic acid of glycine is hydrogen). Exemplary amino acid side chains include, without limitation, methyl (i.e., alanine), sec-buytl (i.e., isoleucine), iso-butyl (i.e., leucine), —CH₂CH₂SCH₃ (i.e., methionine), —CH₂Ph (i.e., phenylalanine),

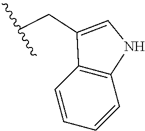 [SMILES: *Cc1c[nH]c2ccccc12]

(i.e., tryptophan),

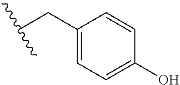 [SMILES: *Cc1ccc(O)cc1]

(i.e., tyrosine), iso-propyl (i.e., valine), hydroxymethyl (i.e., serine), —CH(OH)CH₃ (i.e., threonine), —CH₂C(O)NH₂ (i.e., asparagine), —CH₂CH₂C(O)NH₂ (i.e., glutamine), —CH₂SH (i.e., cysteine), —CH₂SeH (i.e., selenocysteine), —CH₂NH₂ (i.e., glycine), propylene or —CH₂CH₂CH₂— (i.e., proline), —CH₂CH₂CH₂NHC(═NH)NH₂ (i.e., arginine),

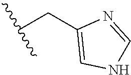 [SMILES: *Cc1c[nH]cn1]

(i.e., histidine), —CH₂CH₂CH₂CH₂NH₂ (i.e., lysine), —CH₂COOH (i.e., aspartic acid), and —CH₂CH₂COOH (i.e., glutamic acid).

In certain embodiments, the biologically active compound (D*) including amide functionality (D*-NHC(O)—R), for example at R⁷, is a prodrug compound of Formula Ia

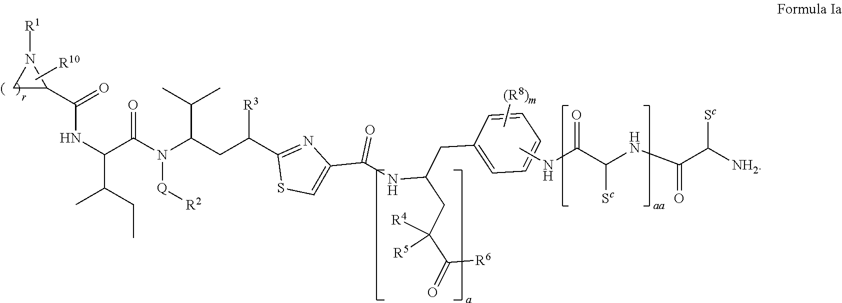

In certain embodiments, prodrug Formula Iaa

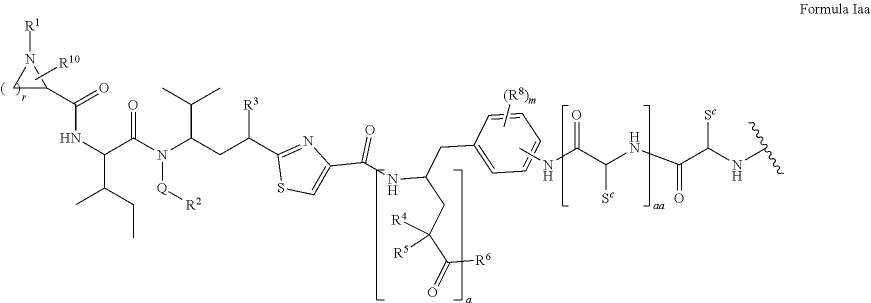

can be linked to a linker or binding agent, as described elsewhere herein, wherein indicates an attachment to the linker, and/or binding agent, as described elsewhere herein.

[symbol]

In certain embodiments, the compounds can be delivered to cells as part of a conjugate. In certain embodiments, the compounds are capable of carrying out any activity of tubulysin or a tubulysin derivative at or in a target, for instance, a target cell. Certain compounds can have one or more additional activities. In certain embodiments, the compounds are capable of modulating the activity of a folate receptor, a somatostatin receptor, and/or a bombesin receptor.

### Compounds, Payloads, or Prodrug Payloads-Q is Carbon

In certain embodiments, set forth herein is a compound having the structure of Formula I, wherein r is four.

In certain embodiments of Formula I above, useful R³ groups include hydroxyl, —O—C₁-C₅ alkyl, —OC(O)C₁-C₅ alkyl, —OC(O)N(H)C₁-C₁₀ alkyl, —OC(O)N(H)C₁-C₁₀ alkyl-NR^{3a}R^{3b}, —NHC(O)C₁-C₅ alkyl, or —OC(O)N(H)(CH₂CH₂O)ₙC₁-C₁₀ alkyl-NR^{3a}R^{3b}, wherein R^{3a} and R^{3b} are independently in each instance, hydrogen, alkyl, alkenyl, alkynyl, cycloalkyl, aryl, heteroaryl, and acyl, wherein alkyl, alkenyl, alkynyl, cycloalkyl, aryl, heteroaryl, and acyl are optionally substituted. In one embodiment, R³ is hydroxyl. In one embodiment, R³ is —O—C₁-C₅ alkyl. In one embodiment, R³ is —OMe. In one embodiment, R³ is-OEt. In one embodiment, R³ is —O-propyl, and constitutional isomers thereof and constitutional isomers thereof. In one embodiment, R³ is —O-butyl, and constitutional isomers thereof. In one embodiment, R³ is —O-pentyl, and constitutional isomers thereof. In one embodiment, R³ is —OC(O)C₁-C₅ alkyl. In one embodiment, R³ is —OC(O)Me. In one embodiment, R³ is —OC(O)Et. In one embodiment, R³ is —OC(O)-propyl, and constitutional isomers thereof. In one embodiment, R³ is —OC(O)-butyl, and constitutional isomers thereof. In one embodiment, R³ is —OC(O)-pentyl, and constitutional isomers thereof. In one embodiment, R³ is —OC(O)N(H)C₁-C₁₀ alkyl. In one embodiment, R³ is —OC(O)N(H)Me. In one embodiment, R³ is —OC(O)N(H)Et. In one embodiment, R³ is —OC(O)N(H)-propyl, and constitutional isomers thereof. In one embodiment, R³ is —OC(O)N(H)-butyl, and constitutional isomers thereof. In one embodiment, R³ is —OC(O)N(H)-pentyl, and constitutional isomers thereof. In one embodiment, R³ is —OC(O)N(H)-hexyl, and constitutional isomers thereof. In one embodiment, R³ is —OC(O)N(H)-heptyl, and constitutional isomers thereof. In one embodiment, R³ is —OC(O)N(H)-octyl, and constitutional isomers thereof. In one embodiment, R³ is —OC(O)N(H)-nonyl, and constitutional isomers thereof. In one embodiment, R³ is —OC(O)N(H)-decyl, and constitutional isomers thereof. In one embodiment, R³ is —OC(O)N(H)C₁-C₁₀ alkyl-NR^{3a}R^{3b}. In one embodiment, R³ is —OC(O)N(H)CH₂NR^{3a}R^{3b}. In one embodiment, R³ is —OC(O)N(H)CH₂CH₂NR^{3a}R^{3b}. In one embodiment, R³ is —OC(O)N(H)CH₂CH₂CH₂NR^{3a}R^{3b}. In one embodiment, R³ is —OC(O)N(H)CH₂CH₂CH₂CH₂NR^{3a}R^{3b}. In one embodiment, R³ is —OC(O)N(H)CH₂CH₂CH₂CH₂CH₂NR^{3a}R^{3b}. In one embodiment, R³ is —OC(O)N(H)CH₂CH₂CH₂CH₂CH₂CH₂NR^{3a}R^{3b}. In one embodiment, R³ is —OC(O)N(H)CH₂CH₂CH₂CH₂CH₂CH₂CH₂NR^{3a}R^{3b}. In one embodiment, R³ is —OC(O)N(H)CH₂CH₂CH₂CH₂CH₂CH₂CH₂CH₂NR^{3a}R^{3b}. In one embodiment, R³ is —OC(O)N(H)CH₂CH₂CH₂CH₂CH₂CH₂CH₂CH₂CH₂NR^{3a}R^{3b}. In one embodiment, R³ is —OC(O)N(H)CH₂CH₂CH₂CH₂CH₂CH₂CH₂CH₂CH₂CH₂NR^{3a}R^{3b}. In any of the immediately preceding eleven embodiments, R^{3a} and R^{3b} are hydrogen. In one embodiment, R³ is —NHC(O)C₁-C₅ alkyl. In one embodiment, R³ is —NHC(O)Me. In one embodiment, R³ is —NHC(O)Et. In one embodiment, R³ is —NHC(O)-propyl, and constitutional isomers thereof. In one embodiment, R³ is —NHC(O)-butyl, and constitutional isomers thereof. In one embodiment, R³ is —NHC(O)-pentyl, and constitutional isomers thereof. In one embodiment, R³ is —OC(O)N(H)(CH₂CH₂O)ₙC₁-C₁₀ alkyl-NR^{3a}R^{3b}, wherein n is an integer from one to ten. In one embodiment, R³ is —OC(O)N(H)(CH₂CH₂O)ₙCH₂NR^{3a}R^{3b}, wherein n is an integer from one to ten. In one embodiment, R³ is —OC(O)N(H)(CH₂CH₂O)ₙCH₂CH₂NR^{3a}R^{3b}, wherein n is an integer from one to ten. In one embodiment, R³ is —OC(O)N(H)(CH₂CH₂O)ₙCH₂CH₂NR^{3a}R^{3b}, wherein n is three. In one embodiment, R³ is —OC(O)N(H)(CH₂CH₂O)ₙCH₂CH₂CH₂NR^{3a}R^{3b}, wherein n is an integer from one to ten. In one embodiment, R³ is —OC(O)N(H)(CH₂CH₂O)ₙCH₂CH₂CH₂CH₂NR^{3a}R^{3b}, wherein n is an integer from one to ten. In one embodiment, R³ is —OC(O)N(H)(CH₂CH₂O)ₙCH₂CH₂CH₂CH₂CH₂NR^{3a}R^{3b}, wherein n is an integer from one to ten. In one embodiment, R³ is —OC(O)N(H)(CH₂CH₂O)ₙCH₂CH₂CH₂CH₂CH₂CH₂NR^{3a}R^{3b}, wherein n is an integer from one to ten. In one embodiment, R³ is —OC(O)N(H)(CH₂CH₂O)ₙCH₂CH₂CH₂CH₂CH₂CH₂CH₂NR^{3a}R^{3b}, wherein n is an integer from one to ten. In one embodiment, R³ is —OC(O)N(H)(CH₂CH₂O)ₙCH₂CH₂CH₂CH₂CH₂CH₂CH₂CH₂NR^{3a}R^{3b}, wherein n is an integer from one to ten. In one embodiment, R³ is —OC(O)N(H)(CH₂CH₂O)ₙCH₂CH₂CH₂CH₂CH₂CH₂CH₂CH₂CH₂NR^{3a}R^{3b}, wherein n is an integer from one to ten. In one embodiment, R³ is —OC(O)N(H)(CH₂CH₂O)ₙCH₂CH₂CH₂CH₂CH₂CH₂CH₂CH₂CH₂CH₂NR^{3a}R^{3b}, wherein n is an integer from one to ten. In any of the immediately preceding twelve embodiments, R^{3a} and R^{3b} are hydrogen.

In certain embodiments of Formula I above, useful R⁷ groups independently include hydrogen, —OH, fluoro, chloro, bromo, iodo, and —NR^{7a}R^{7b}. In one embodiment, R⁷ is hydrogen. In one embodiment, R⁷ is —OH. In one embodiment, R⁷ is fluoro. In another embodiment, R⁷ is chloro. In another embodiment, R⁷ is bromo. In another embodiment, R⁷ is iodo. In one embodiment, R⁷ is —NR^{7a}R^{7b}. In one embodiment, R^{7a} and R^{7b} are hydrogen. In one embodiment, R^{7a} is hydrogen and R^{7b} is —C(O)CH₂OH. In one embodiment, R^{7a} is hydrogen and R^{7b} is a first N-terminal amino acid residue. R^{7b} as a first N-terminal amino acid residue distinguishes these amino acid residues from second amino acid residues within the linker, as described elsewhere herein. In one embodiment, R^{7a} is hydrogen and R^{7b} is a first N-terminal peptide residue. R^{7b} as a first N-terminal peptide residue distinguishes these peptide residues from second peptide residues within the linker, as described elsewhere herein. In one embodiment, R^{7a} is hydrogen and R^{7b} is —CH₂CH₂NH₂.

In certain embodiments of Formula I above, useful R⁸ groups independently include hydrogen, —NHR⁹, and halogen. In one embodiment, R⁸ is hydrogen. In one embodiment, R⁸ is —NHR⁹, wherein R⁹ is hydrogen. In one embodiment, R⁸ is fluoro. In another embodiment, R⁸ is chloro. In another embodiment, R⁸ is bromo. In another embodiment, R⁸ is iodo. In one embodiment, m is one. In one embodiment, m is two.

In certain embodiments, set forth herein is a compound having the structure of Formula I

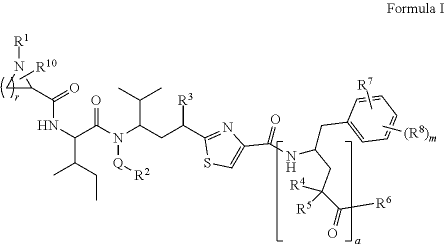

or a pharmaceutically acceptable salt or prodrug thereof, wherein Q is —CH₂—; R¹ is C₁-C₁₀ alkyl; R² is alkyl; R⁴ and R⁵ are C₁-C₅ alkyl; R⁶ is —OH; R⁰ is absent; wherein r is four; and wherein a is one. In Formula I, in certain embodiments, useful R¹ groups include methyl and ethyl. In certain embodiments, useful R¹ groups include propyl, butyl, pentyl, hexyl, heptyl, octyl, nonyl, decyl, and constitutional isomers thereof. In one embodiment, R¹ is methyl. In one embodiment, R¹ is ethyl. In one embodiment, R¹ is propyl, and constitutional isomers thereof. In one embodiment, R¹ is butyl, and constitutional isomers thereof. In one embodiment, R¹ is pentyl, and constitutional isomers thereof. In one embodiment, R¹ is hexyl, and constitutional isomers thereof. In one embodiment, R¹ is heptyl, and constitutional isomers thereof. In one embodiment, R¹ is octyl, and constitutional isomers thereof. In one embodiment, R¹ is nonyl, and constitutional isomers thereof. In one embodiment, R¹ is decyl, and constitutional isomers thereof. In Formula I, in certain embodiments above, useful R² groups include n-pentyl, n-hexyl, n-heptyl, n-octyl, n-nonyl, and n-decyl. In one embodiment, R² is n-pentyl, or constitutional isomers thereof. In another embodiment, R² is n-hexyl, or constitutional isomers thereof. In another embodiment, R² is n-heptyl, or constitutional isomers thereof. In another embodiment, R² is n-octyl, or constitutional isomers thereof. In another embodiment, R² is n-nonyl, or constitutional isomers thereof. In another embodiment, R² is n-decyl, or constitutional isomers thereof. In one embodiment, Q-R² is n-hexyl. In Formula I, in certain embodiments, useful R³ groups are as described above. In certain embodiments of Formula I above, useful R⁴ groups include methyl, ethyl, propyl, butyl, and pentyl. In one embodiment, R⁴ is methyl. In another embodiment, R⁴ is ethyl. In another embodiment, R⁴ is propyl, and constitutional isomers thereof. In another embodiment, R⁴ is butyl, and constitutional isomers thereof. In another embodiment, R⁴ is pentyl, and constitutional isomers thereof. In certain embodiments of Formula I above, useful R⁵ groups include methyl, ethyl, propyl, butyl, and pentyl. In one embodiment, R⁵ is methyl. In another embodiment, R⁵ is ethyl. In another embodiment, R⁵ is propyl, and constitutional isomers thereof. In another embodiment, R⁵ is butyl, and constitutional isomers thereof. In another embodiment, R⁵ is pentyl, and constitutional isomers thereof. In certain embodiments of Formula I above, independent combinations of R⁴ and R⁵ are contemplated herein. For example, in one embodiment, R⁴ and R⁵ are methyl. In one embodiment, R⁴ and R⁵ are ethyl. In one embodiment, R⁴ and R⁵ are, independently, propyl and constitutional isomers. In one embodiment, R⁴ and R⁵ are, independently, butyl and constitutional isomers. In one embodiment, R⁴ and R⁵ are, independently, pentyl and constitutional isomers. In one embodiment, R⁴ is ethyl and R⁵ is methyl. In one embodiment, R⁴ is ethyl and R⁵ is, independently, propyl and constitutional isomers thereof. In one embodiment, R⁴ is, independently, propyl and constitutional isomers thereof; and R⁵ is, independently, butyl and constitutional isomers thereof. In one embodiment, R⁴ is, independently, butyl and constitutional isomers thereof; and R⁵ is, independently, pentyl and constitutional isomers thereof.

In certain embodiments, set forth herein is a compound having the structure of Formula II

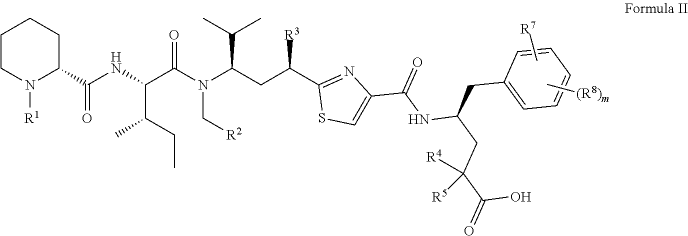

or a pharmaceutically acceptable salt or prodrug thereof. In certain embodiments, R¹, R², R³, R⁴, R⁵, R⁷, R⁸, and m are as described in the context of Formula I, above. In certain embodiments, R³ is hydroxyl,-OEt, —OC(O)N(H)CH₂CH₂NH₂, —NHC(O)Me, or —OC(O)N(H)CH₂CH₂OCH₂CH₂OCH₂CH₂OCH₂CH₂NH₂. In one embodiment, R³ is hydroxyl. In one embodiment, R³ is-OEt. In one embodiment, R³ is —OC(O)N(H)CH₂CH₂NH₂. In one embodiment, R³ is —NHC(O)Me. In one embodiment, R³ is —OC(O)N(H)CH₂CH₂OCH₂CH₂OCH₂CH₂OCH₂CH₂NH₂.

In certain embodiments, provided herein are compounds according to Formula II, selected from the group consisting of

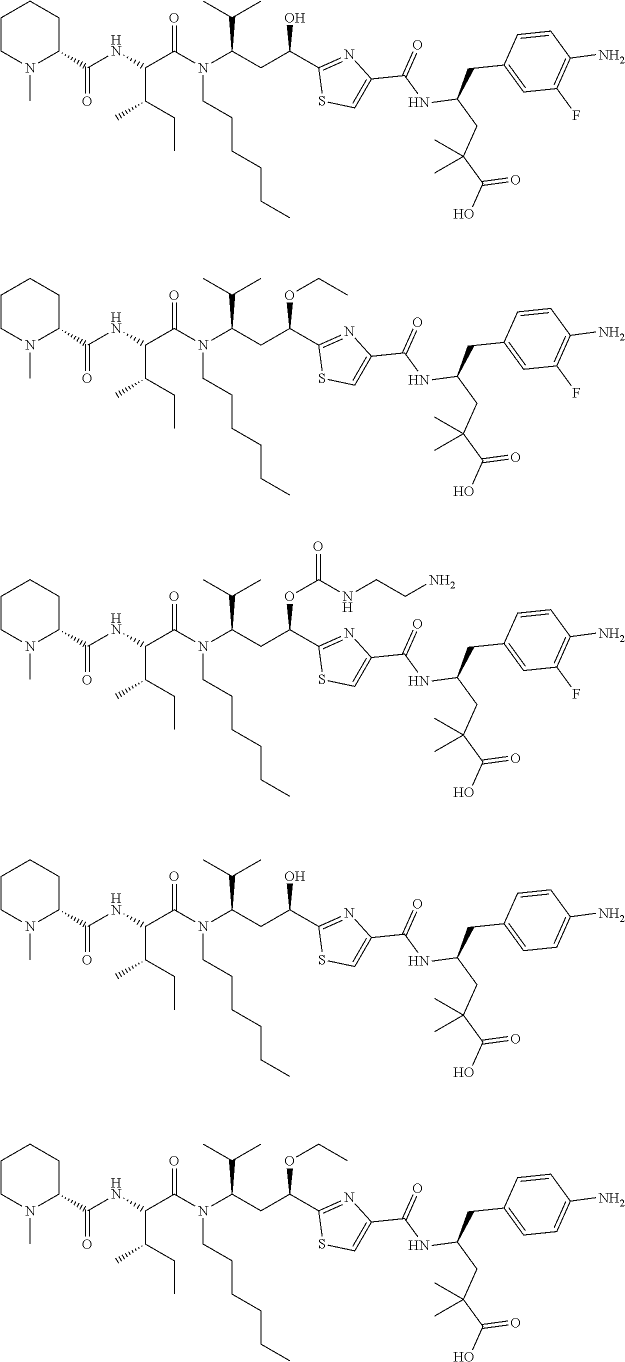

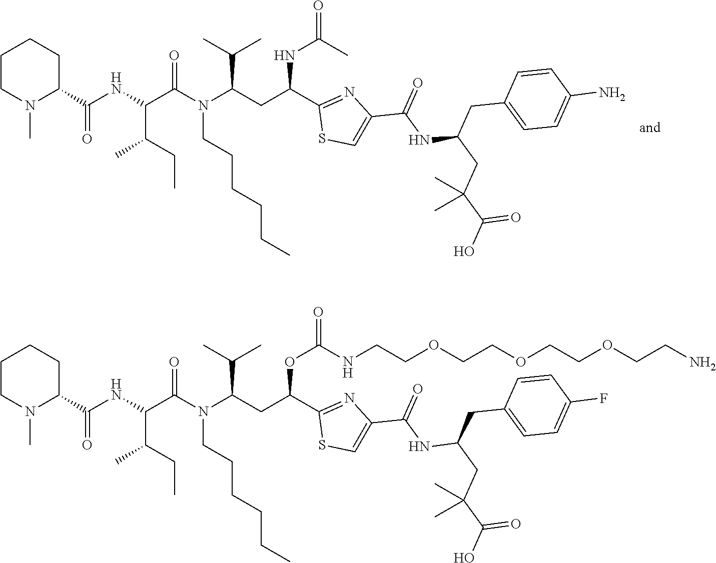

or
a pharmaceutically acceptable salt thereof.

In certain embodiments, set forth herein is a compound having the structure of Formula I

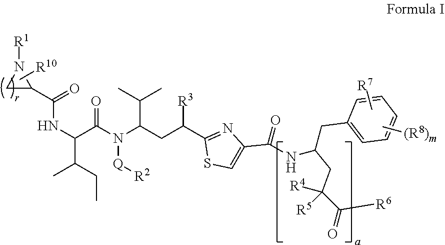

or a pharmaceutically acceptable salt or prodrug thereof, wherein Q is —CH₂—; R¹ is hydrogen or C₁-C₁₀ alkyl; R² is alkyl; R⁴ and R⁵ are C₁-C₅ alkyl; R⁶ is —OH; wherein r is three or four; and wherein a is one. In Formula I, in one embodiment, R¹ is hydrogen. In Formula I, in certain embodiments, useful R¹ groups include methyl and ethyl. In certain embodiments, useful R¹ groups include propyl, butyl, pentyl, hexyl, heptyl, octyl, nonyl, decyl, and constitutional isomers thereof. In one embodiment, R¹ is methyl. In one embodiment, R¹ is ethyl. In one embodiment, R¹ is propyl, and constitutional isomers thereof. In one embodiment, R¹ is butyl, and constitutional isomers thereof. In one embodiment, R¹ is pentyl, and constitutional isomers thereof. In one embodiment, R¹ is hexyl, and constitutional isomers thereof. In one embodiment, R¹ is heptyl, and constitutional isomers thereof. In one embodiment, R¹ is octyl, and constitutional isomers thereof. In one embodiment, R¹ is nonyl, and constitutional isomers thereof. In one embodiment, R¹ is decyl, and constitutional isomers thereof. In Formula I, in certain embodiments above, useful R² groups include n-pentyl, n-hexyl, n-heptyl, n-octyl, n-nonyl, and n-decyl. In one embodiment, R² is n-pentyl, or constitutional isomers thereof. In another embodiment, R² is n-hexyl, or constitutional isomers thereof. In another embodiment, R² is n-heptyl, or constitutional isomers thereof. In another embodiment, R² is n-octyl, or constitutional isomers thereof. In another embodiment, R² is n-nonyl, or constitutional isomers thereof. In another embodiment, R² is n-decyl, or constitutional isomers thereof. In one embodiment, Q-R² is n-hexyl. In Formula I, in certain embodiments, useful R³ groups are as described above. In certain embodiments of Formula I above, useful R⁴ groups include methyl, ethyl, propyl, butyl, and pentyl. In one embodiment, R⁴ is methyl. In another embodiment, R⁴ is ethyl. In another embodiment, R⁴ is propyl, and constitutional isomers thereof. In another embodiment, R⁴ is butyl, and constitutional isomers thereof. In another embodiment, R⁴ is pentyl, and constitutional isomers thereof. In certain embodiments of Formula I above, useful R⁵ groups include methyl, ethyl, propyl, butyl, and pentyl. In one embodiment, R⁵ is methyl. In another embodiment, R⁵ is ethyl. In another embodiment, R⁵ is propyl, and constitutional isomers thereof. In another embodiment, R⁵ is butyl, and constitutional isomers thereof. In another embodiment, R⁵ is pentyl, and constitutional isomers thereof. In certain embodiments of Formula I above, independent combinations of R⁴ and R⁵ are contemplated herein. For example, in one embodiment, R⁴ and R⁵ are methyl. In one embodiment, R⁴ and R⁵ are ethyl. In one embodiment, R⁴ and R⁵ are, independently, propyl and constitutional isomers. In one embodiment, R⁴ and R⁵ are, independently, butyl and constitutional isomers. In one embodiment, R⁴ and R⁵ are, independently, pentyl and constitutional isomers. In one embodiment, R⁴ is ethyl and R⁵ is methyl. In one embodiment, R⁴ is ethyl and R⁵ is, independently, propyl and constitutional isomers thereof. In one embodiment, R⁴ is, independently, propyl and constitutional isomers thereof; and R⁵ is, independently, butyl and constitutional isomers thereof. In one embodiment, R⁴ is, independently, butyl and constitutional isomers thereof; and R⁵ is, independently, pentyl and constitutional isomers thereof. In Formula I, in certain embodiments, useful R⁷ and R⁸ groups are as described above. In certain embodiments of Formula I, R¹⁰ is —C₁-C₅ alkyl. In certain embodiments, useful R¹⁰ groups include propyl, butyl, pentyl, hexyl, heptyl, octyl, nonyl, decyl, and constitutional isomers thereof. In one embodiment, R¹⁰ is methyl. In one embodiment, R¹⁰ is ethyl. In one embodiment, R¹⁰ is propyl, and constitutional isomers thereof. In one embodiment, R¹⁰ is butyl, and constitutional isomers thereof. In one embodiment, R¹⁰ is pentyl, and constitutional isomers thereof. In one embodiment, R¹⁰ is hexyl, and constitutional isomers thereof. In one embodiment, R¹⁰ is heptyl, and constitutional isomers thereof. In one embodiment, R¹⁰ is octyl, and constitutional isomers thereof. In one embodiment, R¹⁰ is nonyl, and constitutional isomers thereof. In one embodiment, R¹⁰ is decyl, and constitutional isomers thereof. In one embodiment, r is three. In one embodiment, r is four.

In certain embodiments, set forth herein is a compound having the structure of Formula III

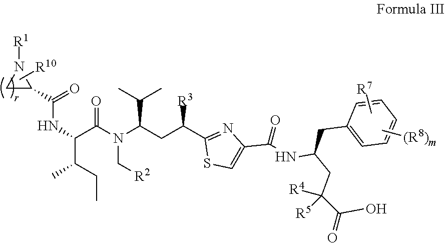

or a pharmaceutically acceptable salt or prodrug thereof. In certain embodiments, R¹, R², R³, R⁴, R⁵, R⁷, R⁸, R¹⁰, and m are as described in the context of Formula I, above. In certain embodiments, R¹ is hydrogen or methyl; and R¹⁰ is methyl. In one embodiment, R¹ is hydrogen; and R¹⁰ is methyl. In one embodiment, R¹ is methyl; and R¹⁰ is methyl.

In certain embodiments, provided herein are compounds according to Formula III, selected from the group consisting of

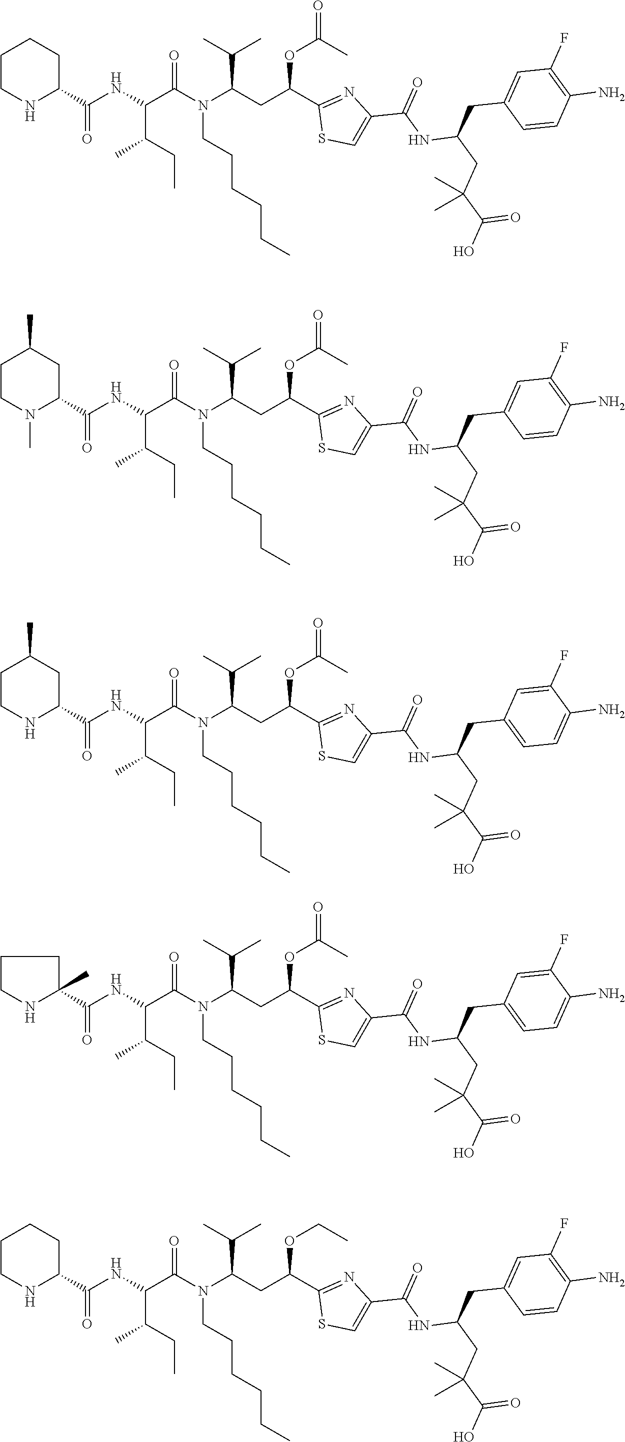

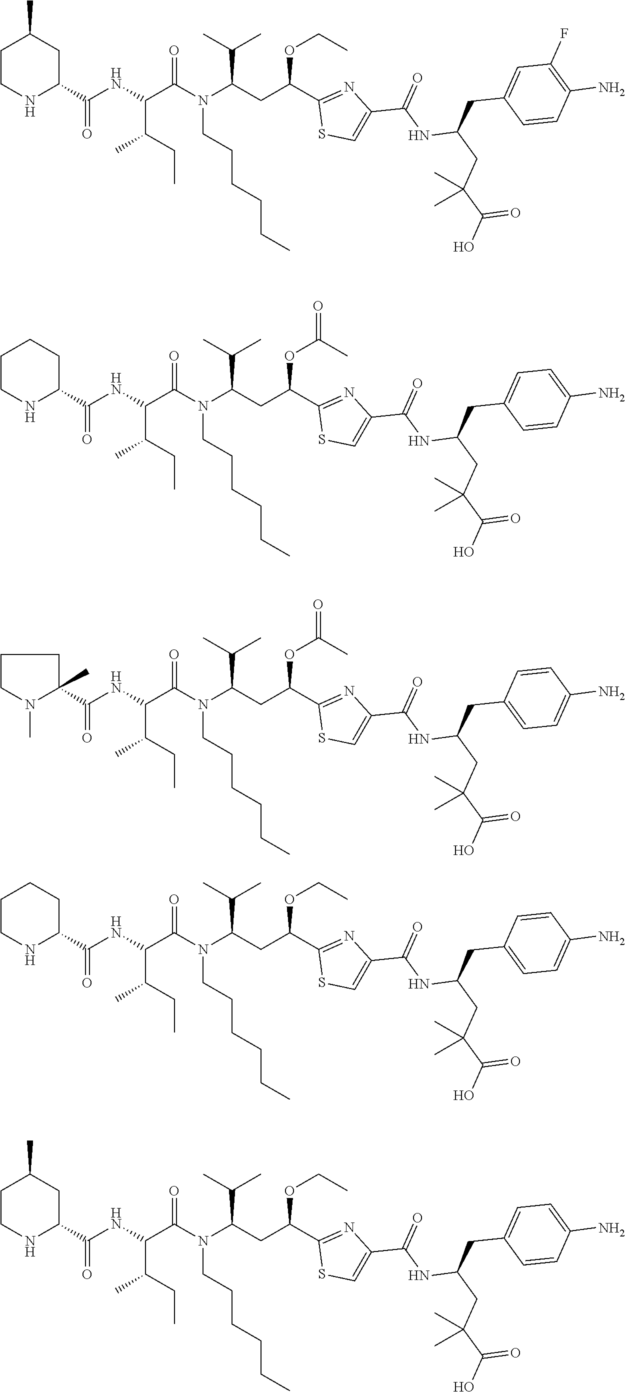

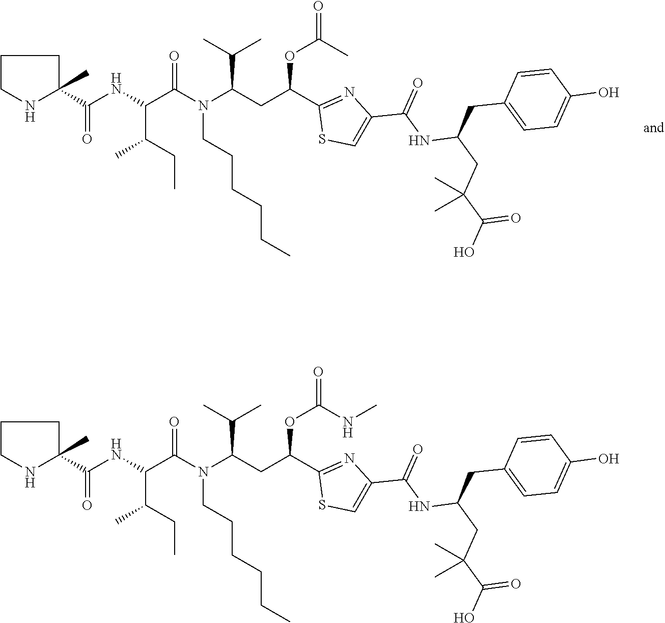

or
a pharmaceutically acceptable salt thereof.

In certain embodiments, set forth herein is a compound having the structure of Formula I

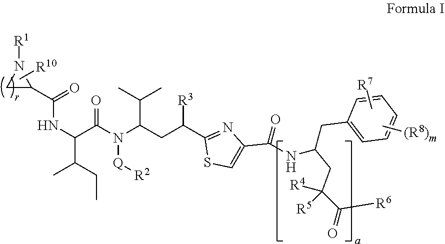

or a pharmaceutically acceptable salt or prodrug thereof, wherein Q is —CH₂—; R¹ is hydrogen or C₁-C₁₀ alkyl; R² is alkyl; R⁴ and R⁵ are C₁-C₅ alkyl; R⁶ is —OH; R¹⁰ is absent; wherein r is four; and wherein a is one. In Formula I, in one embodiment, R¹ is hydrogen. In Formula I, in certain embodiments, useful R¹ groups include methyl and ethyl. In certain embodiments, useful R¹ groups include propyl, butyl, pentyl, hexyl, heptyl, octyl, nonyl, decyl, and constitutional isomers thereof. In one embodiment, R¹ is methyl. In one embodiment, R¹ is ethyl. In one embodiment, R¹ is propyl, and constitutional isomers thereof. In one embodiment, R¹ is butyl, and constitutional isomers thereof. In one embodiment, R¹ is pentyl, and constitutional isomers thereof. In one embodiment, R¹ is hexyl, and constitutional isomers thereof. In one embodiment, R¹ is heptyl, and constitutional isomers thereof. In one embodiment, R¹ is octyl, and constitutional isomers thereof. In one embodiment, R¹ is nonyl, and constitutional isomers thereof. In one embodiment, R¹ is decyl, and constitutional isomers thereof. In Formula I, in certain embodiments above, useful R² groups include n-pentyl, n-hexyl, n-heptyl, n-octyl, n-nonyl, and n-decyl. In one embodiment, R² is n-pentyl, or constitutional isomers thereof. In another embodiment, R² is n-hexyl, or constitutional isomers thereof. In another embodiment, R² is n-heptyl, or constitutional isomers thereof. In another embodiment, R² is n-octyl, or constitutional isomers thereof. In another embodiment, R² is n-nonyl, or constitutional isomers thereof. In another embodiment, R² is n-decyl, or constitutional isomers thereof. In one embodiment, Q-R² is n-hexyl. In Formula I, in certain embodiments, useful R³ groups are as described above. In certain embodiments of Formula I above, useful R⁴ groups include methyl, ethyl, propyl, butyl, and pentyl. In one embodiment, R⁴ is methyl. In another embodiment, R⁴ is ethyl. In another embodiment, R⁴ is propyl, and constitutional isomers thereof. In another embodiment, R⁴ is butyl, and constitutional isomers thereof. In another embodiment, R⁴ is pentyl, and constitutional isomers thereof. In certain embodiments of Formula I above, useful R⁵ groups include methyl, ethyl, propyl, butyl, and pentyl. In one embodiment, R⁵ is methyl. In another embodiment, R⁵ is ethyl. In another embodiment, R⁵ is propyl, and constitutional isomers thereof. In another embodiment, R⁵ is butyl, and constitutional isomers thereof. In another embodiment, R⁵ is pentyl, and constitutional isomers thereof. In certain embodiments of Formula I above, independent combinations of R⁴ and R⁵ are contemplated herein. For example, in one embodiment, R⁴ and R⁵ are methyl. In one embodiment, R⁴ and R⁵ are ethyl. In one embodiment, R⁴ and R⁵ are, independently, propyl and constitutional isomers. In one embodiment, R⁴ and R⁵ are, independently, butyl and constitutional isomers. In one embodiment, R⁴ and R⁵ are, independently, pentyl and constitutional isomers. In one embodiment, R⁴ is ethyl and R⁵ is methyl. In one embodiment, R⁴ is ethyl and R⁵ is, independently, propyl and constitutional isomers thereof. In one embodiment, R⁴ is, independently, propyl and constitutional isomers thereof; and R⁵ is, independently, butyl and constitutional isomers thereof. In one embodiment, R⁴ is, independently, butyl and constitutional isomers thereof; and R⁵ is, independently, pentyl and constitutional isomers thereof. In Formula I, in certain embodiments, useful R⁷ and R¹ groups are as described above.

In certain embodiments, set forth herein is a compound having the structure of Formula II

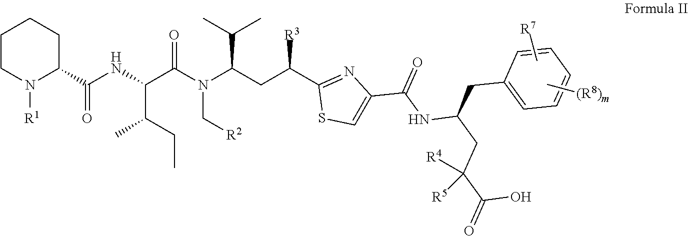

or a pharmaceutically acceptable salt or prodrug thereof. In certain embodiments, R¹, R², R³, R⁴, R⁵, R⁷, R⁸, and m are as described in the context of Formula I, above. In certain embodiments, R⁷ is hydrogen, —N(H)C(O)CH₂NH₂, —N(H)C(O)CH₂OH, or —N(H)CH₂CH₂NH₂; and R⁸ is hydrogen or fluoro. In one embodiment, R⁷ is —N(H)C(O)CH₂NH₂; and R⁸ is fluoro. In one embodiments, R⁷ is —N(H)C(O)CH₂NH₂; and R⁸ is hydrogen. In one embodiments, R⁷ is —N(H)C(O)CH₂OH; and R⁸ is hydrogen. In one embodiments, R⁷ is —N(H)CH₂CH₂NH₂; and R⁸ is hydrogen.

In certain embodiments, provided herein are compounds according to Formula II, selected from the group consisting of

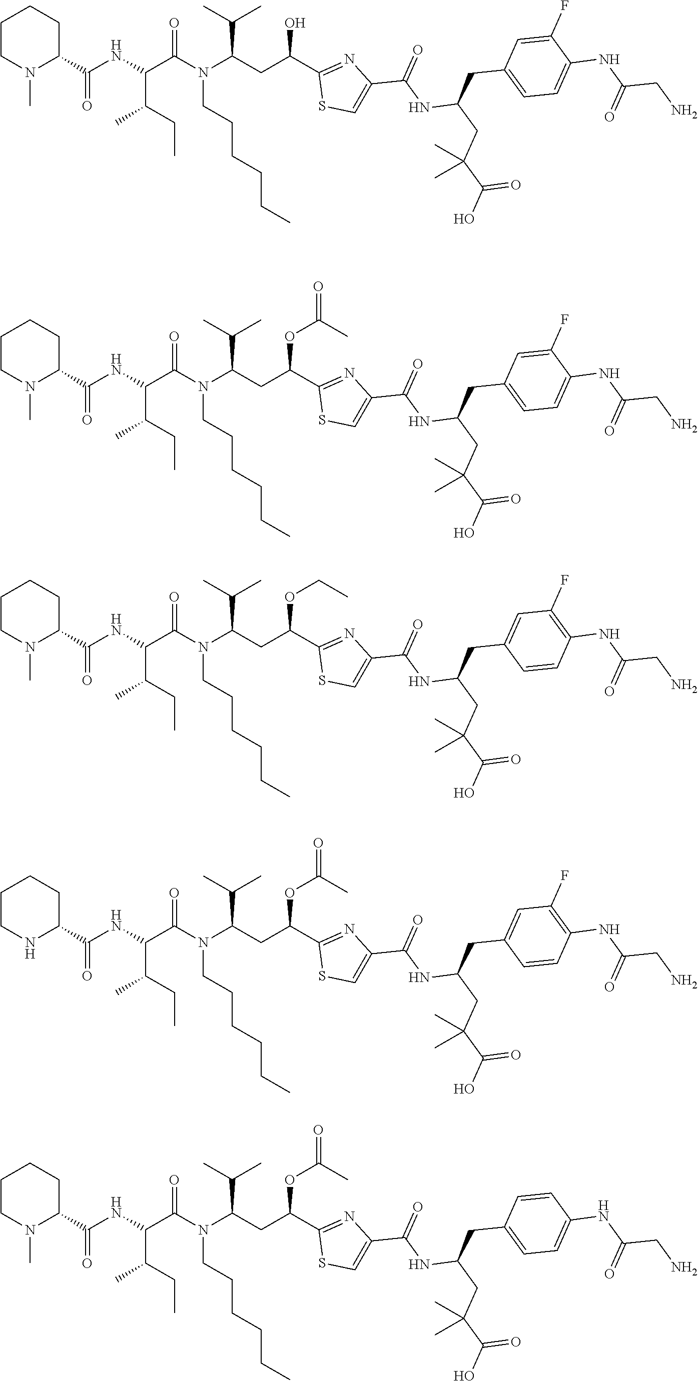

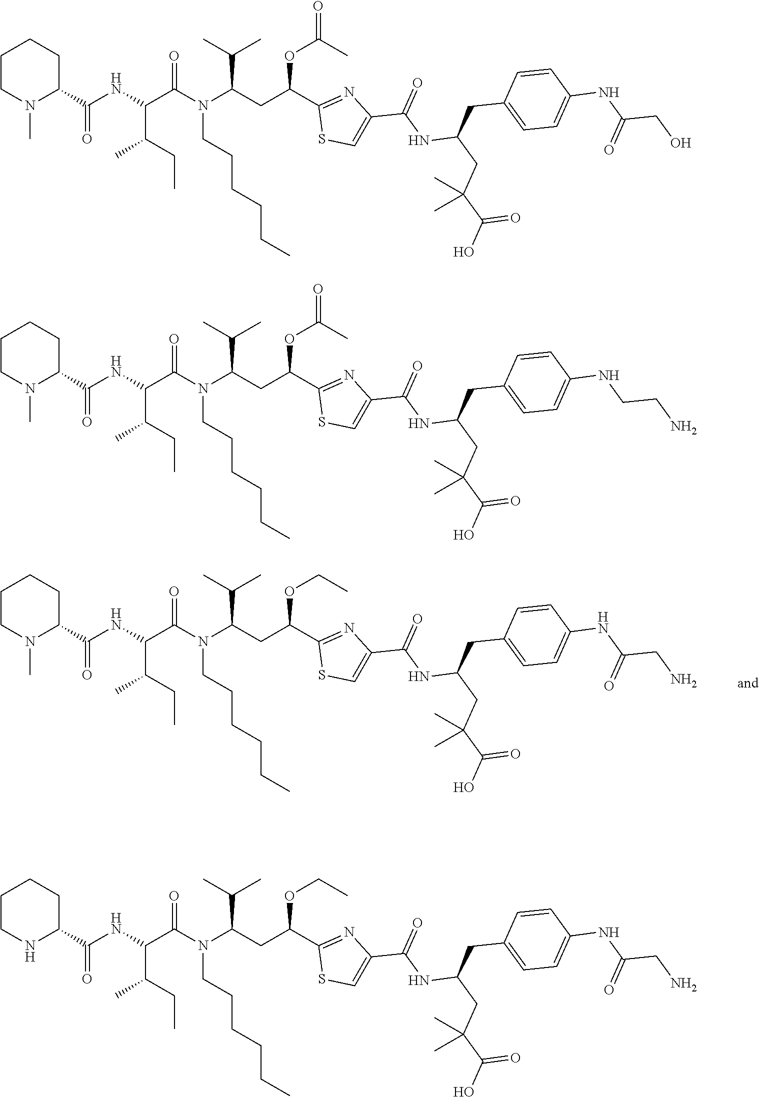

or
a pharmaceutically acceptable salt thereof.

### Compounds, Payloads, or Prodrug Payloads—Q is Oxygen

In certain embodiments, set forth herein is a compound having the structure of Formula I

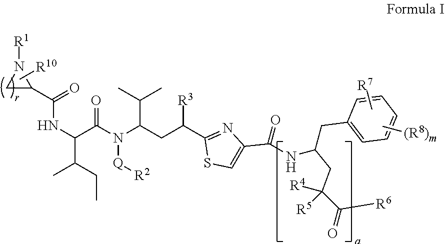

or a pharmaceutically acceptable salt or prodrug thereof, wherein Q is —O—; R¹ is hydrogen or C₁-C₁₀ alkyl; R² is alkyl or alkynyl; R³ is hydroxyl or —OC(O)C₁-C₅ alkyl; R⁴ and R⁵ are C₁-C₅ alkyl; R⁶ is —OH; R⁰, when present, is —C₁-C₅ alkyl; wherein r is three or four; and wherein a is one. In Formula I, in one embodiment, R¹ is hydrogen. In Formula I, in certain embodiments, useful R¹ groups include methyl and ethyl. In certain embodiments, useful R¹ groups include propyl, butyl, pentyl, hexyl, heptyl, octyl, nonyl, decyl, and constitutional isomers thereof. In one embodiment, R¹ is methyl. In one embodiment, R¹ is ethyl. In one embodiment, R¹ is propyl, and constitutional isomers thereof. In one embodiment, R¹ is butyl, and constitutional isomers thereof. In one embodiment, R¹ is pentyl, and constitutional isomers thereof. In one embodiment, R¹ is hexyl, and constitutional isomers thereof. In one embodiment, R¹ is heptyl, and constitutional isomers thereof. In one embodiment, R¹ is octyl, and constitutional isomers thereof. In one embodiment, R¹ is nonyl, and constitutional isomers thereof. In one embodiment, R¹ is decyl, and constitutional isomers thereof. In Formula I, in certain embodiments above, useful R² groups include n-pentyl, n-hexyl, n-heptyl, n-octyl, n-nonyl, and n-decyl. In one embodiment, R² is n-pentyl, or constitutional isomers thereof. In another embodiment, R² is n-hexyl, or constitutional isomers thereof. In another embodiment, R² is n-heptyl, or constitutional isomers thereof. In another embodiment, R² is n-octyl, or constitutional isomers thereof. In another embodiment, R² is n-nonyl, or constitutional isomers thereof. In another embodiment, R² is n-decyl, or constitutional isomers thereof. In one embodiment of Formula I, R² is —CH₂CCH. In one embodiment of Formula I, R² is —CH₂CH₂CCH. In one embodiment of Formula I, R² is —CH₂CH₂CH₂CCH. In one embodiment of Formula I, R² is —CH₂CH₂CH₂CH₂CCH. In one embodiment of Formula I, R² is —CH₂CH₂CH₂CH₂CH₂CCH. In one embodiment of Formula I, R² is —CH₂CH₂CH₂CH₂CH₂CH₂CCH. In one embodiment of Formula I, R² is —CH₂CH₂CH₂CH₂CH₂CH₂CH₂CCH. In one embodiment of Formula I, R² is —CH₂CH₂CH₂CH₂CH₂CH₂CH₂CH₂CCH. In one embodiment of Formula I, R² is —CH₂CH₂CH₂CH₂CH₂CH₂CH₂CH₂CH₂CCH. In one embodiment of Formula I, R² is —CH₂CH₂CH₂CH₂CH₂CH₂CH₂CH₂CH₂CH₂CCH. In one embodiment of Formula I, R³ is hydroxyl. In certain embodiments of Formula I above, useful R³ groups include —C(O)Me, —C(O)Et, —C(O)propyl, —C(O)butyl, and —C(O)pentyl. In one embodiment, R³ is —C(O)Me. In another embodiment, R³ is —C(O)Et. In another embodiment, R³ is —C(O)propyl, and constitutional isomers thereof. In another embodiment, R³ is —C(O)butyl, and constitutional isomers thereof. In another embodiment, R³ is —C(O)pentyl, and constitutional isomers thereof. In certain embodiments of Formula I above, useful R⁴ groups include methyl, ethyl, propyl, butyl, and pentyl. In one embodiment, R⁴ is methyl. In another embodiment, R⁴ is ethyl. In another embodiment, R⁴ is propyl, and constitutional isomers thereof. In another embodiment, R⁴ is butyl, and constitutional isomers thereof. In another embodiment, R⁴ is pentyl, and constitutional isomers thereof. In certain embodiments of Formula I above, useful R⁵ groups include methyl, ethyl, propyl, butyl, and pentyl. In one embodiment, R⁵ is methyl. In another embodiment, R⁵ is ethyl. In another embodiment, R⁵ is propyl, and constitutional isomers thereof. In another embodiment, R⁵ is butyl, and constitutional isomers thereof. In another embodiment, R⁵ is pentyl, and constitutional isomers thereof. In certain embodiments of Formula I above, independent combinations of R⁴ and R⁵ are contemplated herein. For example, in one embodiment, R⁴ and R⁵ are methyl. In one embodiment, R⁴ and R⁵ are ethyl. In one embodiment, R⁴ and R⁵ are, independently, propyl and constitutional isomers. In one embodiment, R⁴ and R⁵ are, independently, butyl and constitutional isomers. In one embodiment, R⁴ and R⁵ are, independently, pentyl and constitutional isomers. In one embodiment, R⁴ is ethyl and R⁵ is methyl. In one embodiment, R⁴ is ethyl and R⁵ is, independently, propyl and constitutional isomers thereof. In one embodiment, R⁴ is, independently, propyl and constitutional isomers thereof; and R⁵ is, independently, butyl and constitutional isomers thereof. In one embodiment, R⁴ is, independently, butyl and constitutional isomers thereof; and R⁵ is, independently, pentyl and constitutional isomers thereof. In Formula I, in certain embodiments, useful R⁷ and R⁸ groups are as described above. In certain embodiments of Formula I, R¹⁰ is absent. In certain embodiments of Formula I, R¹⁰ is —C₁-C₅ alkyl. In certain embodiments, useful R¹⁰ groups include propyl, butyl, pentyl, hexyl, heptyl, octyl, nonyl, decyl, and constitutional isomers thereof. In one embodiment, R¹⁰ is methyl. In one embodiment, R¹⁰ is ethyl. In one embodiment, R¹⁰ is propyl, and constitutional isomers thereof. In one embodiment, R¹⁰ is butyl, and constitutional isomers thereof. In one embodiment, R¹⁰ is pentyl, and constitutional isomers thereof. In one embodiment, R¹⁰ is hexyl, and constitutional isomers thereof. In one embodiment, R¹⁰ is heptyl, and constitutional isomers thereof. In one embodiment, R¹⁰ is octyl, and constitutional isomers thereof. In one embodiment, R¹⁰ is nonyl, and constitutional isomers thereof. In one embodiment, R¹⁰ is decyl, and constitutional isomers thereof. In one embodiment, r is three. In one embodiment, r is four.

In certain embodiments, set forth herein is a compound having the structure of Formula IV

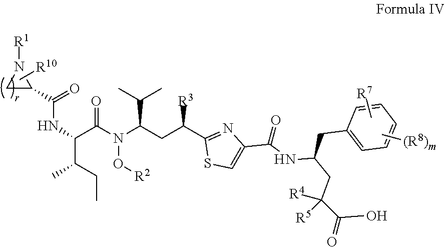

or a pharmaceutically acceptable salt or prodrug thereof. In certain embodiments, R¹, R², R³, R⁴, R⁵, R⁷, R⁸, R¹⁰, and m are as described in the context of Formula I, above. In certain embodiments, R⁷ is hydrogen or —NH₂; and R⁸ is hydrogen or fluoro. In one embodiment, R⁷ is —NH₂; and R⁸ is hydrogen. In one embodiment, R⁷ is —NH₂; and R⁸ is fluoro.

In certain embodiments, provided herein are compounds according to Formula IV, selected from the group consisting of

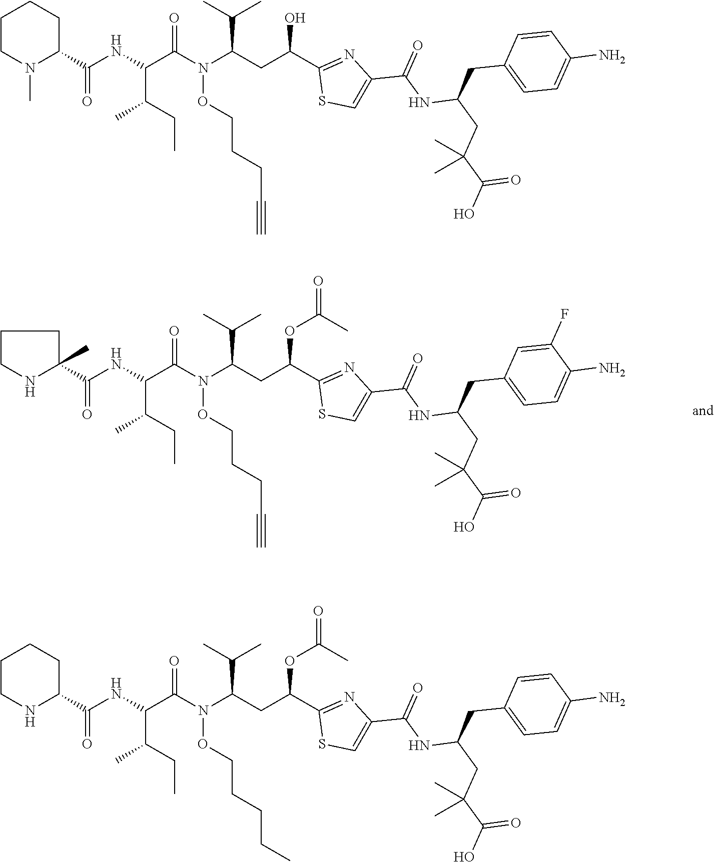

or
a pharmaceutically acceptable salt thereof.

In certain embodiments, set forth herein is a compound having the structure of Formula I

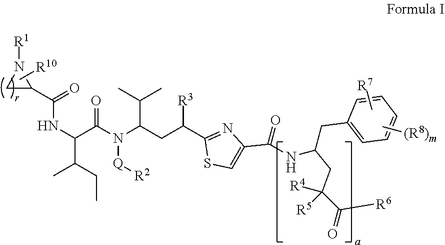

or a pharmaceutically acceptable salt or prodrug thereof, wherein Q is —O—; R¹ is C₁-C₁₀ alkyl; R² is alkynyl; R³ is —OC(O)C₁-C₅ alkyl; R⁴ and R⁵ are C₁-C₅ alkyl; R⁶ is —OH; R⁰ is absent; wherein r is four; and wherein a is one. In Formula I, in certain embodiments, useful R¹ groups include methyl and ethyl. In certain embodiments, useful R¹ groups include propyl, butyl, pentyl, hexyl, heptyl, octyl, nonyl, decyl, and constitutional isomers thereof. In one embodiment, R¹ is methyl. In one embodiment, R¹ is ethyl. In one embodiment, R¹ is propyl, and constitutional isomers thereof. In one embodiment, R¹ is butyl, and constitutional isomers thereof. In one embodiment, R¹ is pentyl, and constitutional isomers thereof. In one embodiment, R¹ is hexyl, and constitutional isomers thereof. In one embodiment, R¹ is heptyl, and constitutional isomers thereof. In one embodiment, R¹ is octyl, and constitutional isomers thereof. In one embodiment, R¹ is nonyl, and constitutional isomers thereof. In one embodiment, R¹ is decyl, and constitutional isomers thereof. In one embodiment of Formula I, R² is —CH₂CCH. In one embodiment of Formula I, R² is —CH₂CH₂CCH. In one embodiment of Formula I, R² is —CH₂CH₂CH₂CCH. In one embodiment of Formula I, R² is —CH₂CH₂CH₂CH₂CCH. In one embodiment of Formula I, R² is —CH₂CH₂CH₂CH₂CH₂CCH. In one embodiment of Formula I, R² is —CH₂CH₂CH₂CH₂CH₂CH₂CCH. In one embodiment of Formula I, R² is —CH₂CH₂CH₂CH₂CH₂CH₂CH₂CCH. In one embodiment of Formula I, R² is —CH₂CH₂CH₂CH₂CH₂CH₂CH₂CH₂CCH. In one embodiment of Formula I, R² is —CH₂CH₂CH₂CH₂CH₂CH₂CH₂CH₂CH₂CCH. In one embodiment of Formula I, R² is —CH₂CH₂CH₂CH₂CH₂CH₂CH₂CH₂CH₂CH₂CCH. In one embodiment of Formula I, R³ is hydroxyl. In certain embodiments of Formula I above, useful R³ groups include —C(O)Me, —C(O)Et, —C(O)propyl, —C(O)butyl, and —C(O)pentyl. In one embodiment, R³ is —C(O)Me. In another embodiment, R³ is —C(O)Et. In another embodiment, R³ is —C(O)propyl, and constitutional isomers thereof. In another embodiment, R³ is —C(O)butyl, and constitutional isomers thereof. In another embodiment, R³ is —C(O)pentyl, and constitutional isomers thereof. In certain embodiments of Formula I above, useful R⁴ groups include methyl, ethyl, propyl, butyl, and pentyl. In one embodiment, R⁴ is methyl. In another embodiment, R⁴ is ethyl. In another embodiment, R⁴ is propyl, and constitutional isomers thereof. In another embodiment, R⁴ is butyl, and constitutional isomers thereof. In another embodiment, R⁴ is pentyl, and constitutional isomers thereof. In certain embodiments of Formula I above, useful R⁵ groups include methyl, ethyl, propyl, butyl, and pentyl. In one embodiment, R⁵ is methyl. In another embodiment, R⁵ is ethyl. In another embodiment, R⁵ is propyl, and constitutional isomers thereof. In another embodiment, R⁵ is butyl, and constitutional isomers thereof. In another embodiment, R⁵ is pentyl, and constitutional isomers thereof. In certain embodiments of Formula I above, independent combinations of R⁴ and R⁵ are contemplated herein. For example, in one embodiment, R⁴ and R⁵ are methyl. In one embodiment, R⁴ and R⁵ are ethyl. In one embodiment, R⁴ and R⁵ are, independently, propyl and constitutional isomers. In one embodiment, R⁴ and R⁵ are, independently, butyl and constitutional isomers. In one embodiment, R⁴ and R⁵ are, independently, pentyl and constitutional isomers. In one embodiment, R⁴ is ethyl and R⁵ is methyl. In one embodiment, R⁴ is ethyl and R⁵ is, independently, propyl and constitutional isomers thereof. In one embodiment, R⁴ is, independently, propyl and constitutional isomers thereof; and R⁵ is, independently, butyl and constitutional isomers thereof. In one embodiment, R⁴ is, independently, butyl and constitutional isomers thereof; and R⁵ is, independently, pentyl and constitutional isomers thereof. In Formula I, in certain embodiments, useful R⁷ and R⁸ groups are as described above.

In certain embodiments, set forth herein is a compound having the structure of Formula V

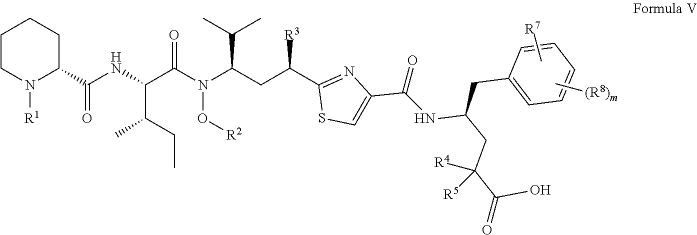

or a pharmaceutically acceptable salt or prodrug thereof. In certain embodiments, R¹, R², R³, R⁴, R⁵, R⁷, R⁸, and m are as described in the context of Formula I, above. In certain embodiments, R⁷ is hydrogen or —N(H)C(O)CH₂OH, —N(H)C(O)CH₂NHC(O)CH₂NH₂, or

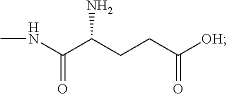

and R⁸ is hydrogen. In one embodiment, R⁷ is —N(H)C(O)CH₂OH; and R⁸ is hydrogen. In one embodiment, R⁷ is —N(H)C(O)CH₂NHC(O)CH₂NH₂; and R⁸ is hydrogen. In one embodiment, R⁷ is

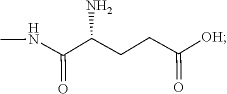

and R⁸ is hydrogen.

In certain embodiments, provided herein are compounds according to Formula V, selected from the group consisting of

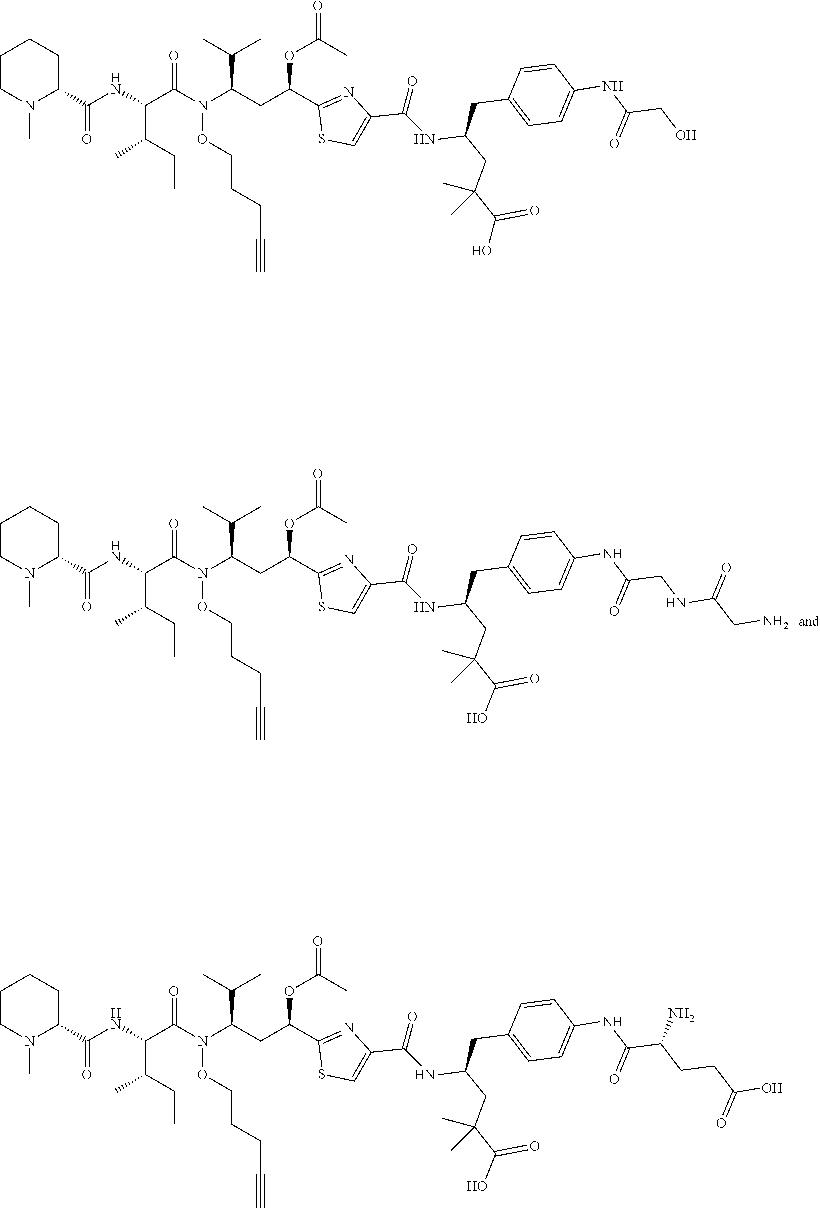 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CO)cc2)CC(C)(C)C(=O)O)cs1)C(C)C ; C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)CN)cc2)CC(C)(C)C(=O)O)cs1)C(C)C ; C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](N)CCC(=O)O)cc2)CC(C)(C)C(=O)O)cs1)C(C)C]

or
a pharmaceutically acceptable salt thereof.

### Compounds, Payloads, or Prodrug Payloads—Q is Carbon or Oxygen

In certain embodiments, set forth herein is a compound having the structure of Formula I

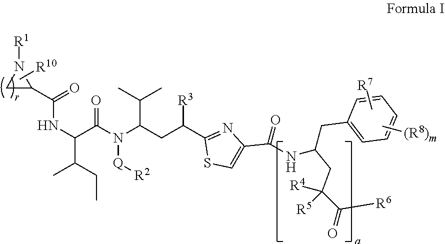

or a pharmaceutically acceptable salt or prodrug thereof, wherein Q is —CH₂— or —O—; R¹ is C₁-C₁₀ alkyl; R² is alkyl or alkynyl; R³; R⁴ and R⁵ are C₁-C₅ alkyl; R⁶ is —NHSO₂(CH₂)ₐ₁-aryl-(CH₂)ₐ₂NR^{6a}R^{6b}; R¹⁰ is absent; wherein r is four; and wherein a, a1, and, a2 are, independently, zero or one. In Formula I, in one embodiment, Q is —CH₂—. In Formula I, in one embodiment Q is —O—. In Formula I, in certain embodiments, useful R¹ groups include methyl and ethyl. In certain embodiments, useful R¹ groups include propyl, butyl, pentyl, hexyl, heptyl, octyl, nonyl, decyl, and constitutional isomers thereof. In one embodiment, R¹ is methyl. In one embodiment, R¹ is ethyl. In one embodiment, R¹ is propyl, and constitutional isomers thereof. In one embodiment, R¹ is butyl, and constitutional isomers thereof. In one embodiment, R¹ is pentyl, and constitutional isomers thereof. In one embodiment, R¹ is hexyl, and constitutional isomers thereof. In one embodiment, R¹ is heptyl, and constitutional isomers thereof. In one embodiment, R¹ is octyl, and constitutional isomers thereof. In one embodiment, R¹ is nonyl, and constitutional isomers thereof. In one embodiment, R¹ is decyl, and constitutional isomers thereof. In Formula I, in certain embodiments above, useful R² groups include n-pentyl, n-hexyl, n-heptyl, n-octyl, n-nonyl, and n-decyl. In one embodiment, R² is n-pentyl, or constitutional isomers thereof. In another embodiment, R² is n-hexyl, or constitutional isomers thereof. In another embodiment, R² is n-heptyl, or constitutional isomers thereof. In another embodiment, R² is n-octyl, or constitutional isomers thereof. In another embodiment, R² is n-nonyl, or constitutional isomers thereof. In another embodiment, R² is n-decyl, or constitutional isomers thereof. In one embodiment of Formula I, R² is —CH₂CCH. In one embodiment of Formula I, R² is —CH₂CH₂CCH. In one embodiment of Formula I, R² is —CH₂CH₂CH₂CCH. In one embodiment of Formula I, R² is —CH₂CH₂CH₂CH₂CCH. In one embodiment of Formula I, R² is —CH₂CH₂CH₂CH₂CH₂CCH. In one embodiment of Formula I, R² is —CH₂CH₂CH₂CH₂CH₂CH₂CCH. In one embodiment of Formula I, R² is —CH₂CH₂CH₂CH₂CH₂CH₂CH₂CCH. In one embodiment of Formula I, R² is —CH₂CH₂CH₂CH₂CH₂CH₂CH₂CH₂CCH. In one embodiment of Formula I, R² is —CH₂CH₂CH₂CH₂CH₂CH₂CH₂CH₂CH₂CCH. In one embodiment of Formula I, R² is —CH₂CH₂CH₂CH₂CH₂CH₂CH₂CH₂CH₂CH₂CCH. In Formula I, in certain embodiments, useful R³ groups are as described above. In certain embodiments of Formula I above, useful R⁴ groups include methyl, ethyl, propyl, butyl, and pentyl. In one embodiment, R⁴ is methyl. In another embodiment, R⁴ is ethyl. In another embodiment, R⁴ is propyl, and constitutional isomers thereof. In another embodiment, R⁴ is butyl, and constitutional isomers thereof. In another embodiment, R⁴ is pentyl, and constitutional isomers thereof. In certain embodiments of Formula I above, useful R⁵ groups include methyl, ethyl, propyl, butyl, and pentyl. In one embodiment, R⁵ is methyl. In another embodiment, R⁵ is ethyl. In another embodiment, R⁵ is propyl, and constitutional isomers thereof. In another embodiment, R⁵ is butyl, and constitutional isomers thereof. In another embodiment, R⁵ is pentyl, and constitutional isomers thereof. In certain embodiments of Formula I above, independent combinations of R⁴ and R⁵ are contemplated herein. For example, in one embodiment, R⁴ and R⁵ are methyl. In one embodiment, R⁴ and R⁵ are ethyl. In one embodiment, R⁴ and R⁵ are, independently, propyl and constitutional isomers. In one embodiment, R⁴ and R⁵ are, independently, butyl and constitutional isomers. In one embodiment, R⁴ and R⁵ are, independently, pentyl and constitutional isomers. In one embodiment, R⁴ is ethyl and R⁵ is methyl. In one embodiment, R⁴ is ethyl and R⁵ is, independently, propyl and constitutional isomers thereof. In one embodiment, R⁴ is, independently, propyl and constitutional isomers thereof; and R⁵ is, independently, butyl and constitutional isomers thereof. In one embodiment, R⁴ is, independently, butyl and constitutional isomers thereof; and R⁵ is, independently, pentyl and constitutional isomers thereof. In Formula I, in certain embodiments, useful R^{6a} and R^{6b} groups are hydrogen. In Formula I, in certain embodiments, a is zero. In Formula I, in certain embodiments, a is one. In Formula I, in certain embodiments, at is zero and a2 is one. In Formula I, in certain embodiments, at is zero and a2 is zero. In Formula I, in certain embodiments, at is one and a2 is zero. In Formula I, in certain embodiments, a is zero, at is zero, and a2 is one. In Formula I, in certain embodiments, a is zero, at is zero, and a2 is zero. In Formula I, in certain embodiments, a is zero, at is one, and a2 is zero. In Formula I, in certain embodiments, a is one, at is zero, and a2 is one. In Formula I, in certain embodiments, a is one, at is zero, and a2 is zero. In Formula I, in certain embodiments, a is one, at is one, and a2 is zero.

In certain embodiments, set forth herein is a compound having the structure of Formula VI

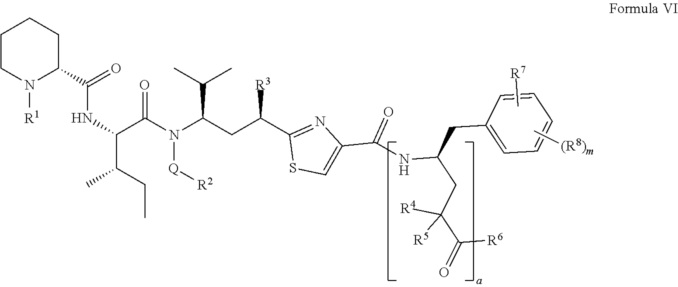

or a pharmaceutically acceptable salt or prodrug thereof. In certain embodiments, Q, R¹, R², R³, R⁴, R⁵, and R⁶ are as described in the context of Formula I, above. In one embodiment, R⁶

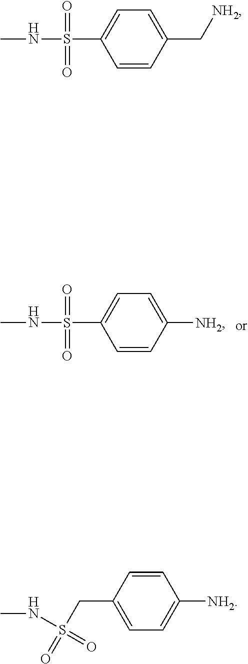

In one embodiment, R⁶ is

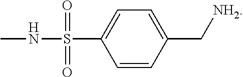

In one embodiment, R⁶ is

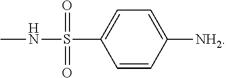

In one embodiment, R⁶ is

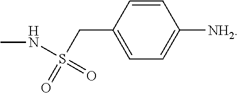

In one embodiment, a is zero; and R⁶ is

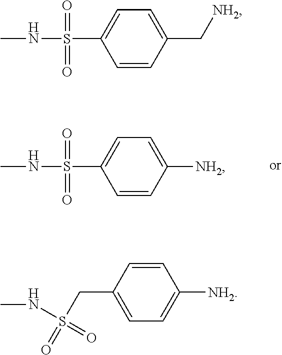

In one embodiment, a is zero; and R⁶ is

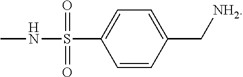

In one embodiment, a is zero; and R⁶ is

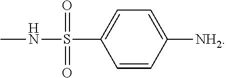

In one embodiment a is zero; and R⁶ is

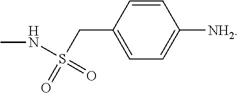

In one embodiment, a is one; and R⁶ is

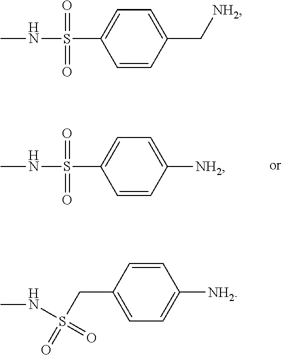

In one embodiment, a is one; and R⁶ is

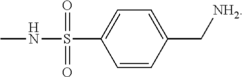

In one embodiment, a is one; and R⁶ is

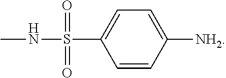

In one embodiment, a is one; and R⁶ is

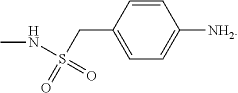

In certain embodiments, provided herein are compounds according to Formula VI, selected from the group consisting of

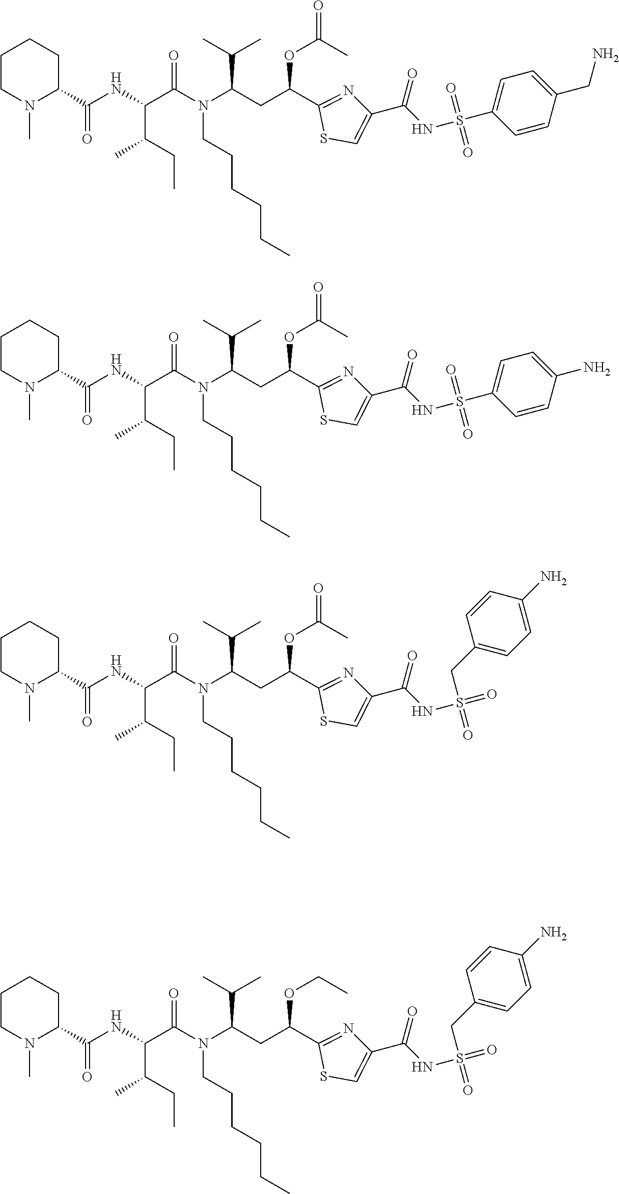

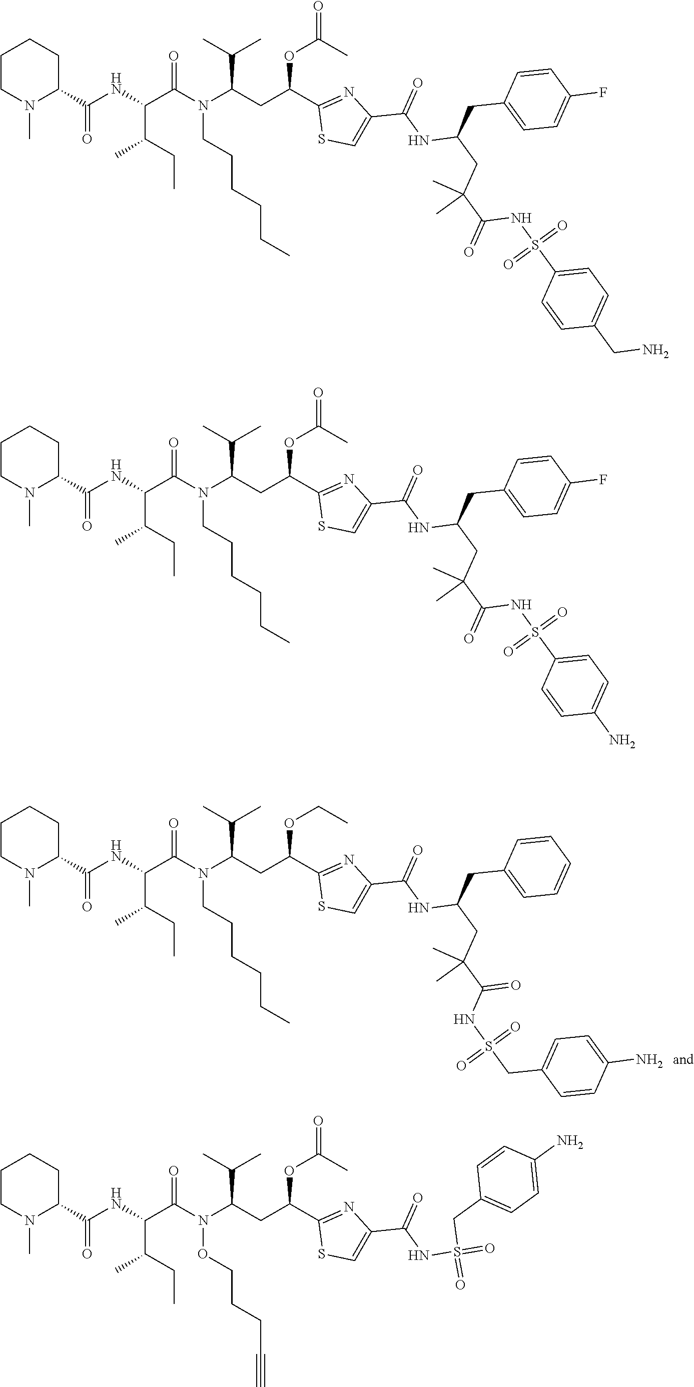

or
a pharmaceutically acceptable salt thereof.

### Binding Agents

Suitable binding agents for any of the conjugates provided in the instant disclosure include, but are not limited to, antibodies, lymphokines (e.g., IL-2 or IL-3), hormones (e.g., insulin and glucocorticoids), growth factors (e.g., EGF, transferrin, and fibronectin type III), viral receptors, interleukins, or any other cell binding or peptide binding molecules or substances. Binding agents also include, but are not limited to, ankyrin repeat proteins and interferons.

In some embodiments, the binding agent is an antibody or an antigen-binding fragment thereof. The antibody can be in any form known to those of skill in the art. The term “antibody,” as used herein, refers to any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2, and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of disclosed herein, the FRs of the antibodies (or antigen-binding portion thereof) suitable for the compounds herein may be identical to the human germline sequences, or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable, standard technique(s) such as proteolytic digestion or recombinant genetic engineering technique(s) involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add, or delete amino acids, etc. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated CDR such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein. An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}-V_{L}, or V_{L}-V_{L} dimers. Alternatively, the antigen-binding fragment of an antibody may contain a monomeric V_{H} or V_{L} domain. In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of this disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-CH2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-CH3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60, or more) amino acids which result in a flexible or semi-flexible linkage between adjacent variable and/or constant domains in a single polypeptide molecule. As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of this disclosure using routine techniques available in the art. In certain embodiments described herein, antibodies described herein are human antibodies. The term “human antibody,” as used herein, is intended to include antibodies having variable and constant regions derived from human germline immunoglobulin sequences. The human antibodies of this disclosure may include amino acid residues not encoded by human germline immunoglobulin sequences (e.g., mutations introduced by random or site-specific mutagenesis in vitro or by somatic mutation in vivo), for example, in the CDRs and in particular CDR3. However, the term “human antibody,” as used herein, is not intended to include antibodies in which CDR sequences derived from the germline of another mammalian species, such as a mouse, have been grafted onto human framework sequences. The term “human antibody” does not include naturally occurring molecules that normally exist without modification or human intervention/manipulation, in a naturally occurring, unmodified living organism. The antibodies disclosed herein may, in some embodiments, be recombinant human antibodies. The term “recombinant human antibody,” as used herein, is intended to include all human antibodies that are prepared, expressed, created, or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created, or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo. Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification. The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant disclosure encompasses antibodies having one or more mutations in the hinge, C_{H}2, or C_{H}3 region which may be desirable, for example, in production, to improve the yield of the desired antibody form. The antibodies described herein may be isolated antibodies. An “isolated antibody,” as used herein, refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the instant disclosure. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals. The antibodies used herein can comprise one or more amino acid substitutions, insertions, and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. This disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FRI or within the last 8 amino acids of FR4, or only the mutated residues found within CDR1, CDR2, or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of this disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within this disclosure. Antibodies useful for the compounds herein also include antibodies comprising variants of any of the HCVR, LCVR, and/or CDR amino acid sequences disclosed herein having one or more conservative substitutions. The term “epitope” refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

In certain embodiments, the antibody comprises a light chain. In certain embodiments, the light chain is a kappa light chain. In certain embodiments, the light chain is a lambda light chain. In certain embodiments, the antibody comprises a heavy chain. In some embodiments, the heavy chain is an IgA. In some embodiments, the heavy chain is an IgD. In some embodiments, the heavy chain is an IgE. In some embodiments, the heavy chain is an IgG. In some embodiments, the heavy chain is an IgM. In some embodiments, the heavy chain is an IgG1. In some embodiments, the heavy chain is an IgG2. In some embodiments, the heavy chain is an IgG3. In some embodiments, the heavy chain is an IgG4. In some embodiments, the heavy chain is an IgA1. In some embodiments, the heavy chain is an IgA2.

In some embodiments, the antibody is an antibody fragment. In some embodiments, the antibody fragment is an Fv fragment. In some embodiments, the antibody fragment is a Fab fragment. In some embodiments, the antibody fragment is a F(ab′)₂ fragment. In some embodiments, the antibody fragment is a Fab′ fragment. In some embodiments, the antibody fragment is an scFv (sFv) fragment. In some embodiments, the antibody fragment is an scFv-Fc fragment.

In some embodiments, the antibody is a monoclonal antibody. In some embodiments, the antibody is a polyclonal antibody. In some embodiments, the antibody is a bispecific antibody including a first antigen-binding domain (also referred to herein as “D1”), and a second antigen-binding domain (also referred to herein as “D2”).

As used herein, the expression “antigen-binding domain” means any peptide, polypeptide, nucleic acid molecule, scaffold-type molecule, peptide display molecule, or polypeptide-containing construct that is capable of specifically binding a particular antigen of interest (e.g., PRLR or STEAP2). The term “specifically binds” or the like, as used herein, means that the antigen-binding domain forms a complex with a particular antigen characterized by a dissociation constant (K_{D}) of 1 μM or less, and does not bind other unrelated antigens under ordinary test conditions. “Unrelated antigens” are proteins, peptides, or polypeptides that have less than 95% amino acid identity to one another.

Exemplary categories of antigen-binding domains that can be used in the context of this disclosure include antibodies, antigen-binding portions of antibodies, peptides that specifically interact with a particular antigen (e.g., peptibodies), receptor molecules that specifically interact with a particular antigen, proteins comprising a ligand-binding portion of a receptor that specifically binds a particular antigen, antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, 2011, Curr. Opin. Biotechnol. 22:849-857, and references cited therein]), and aptamers or portions thereof.

Methods for determining whether two molecules specifically bind one another are well known in the art and include, for example, equilibrium dialysis, surface plasmon resonance, and the like. For example, an antigen-binding domain, as used in the context of this disclosure, includes polypeptides that bind a particular antigen (e.g., a target molecule [T] or an internalizing effector protein [E]) or a portion thereof with a K_{D} of less than about 1 μM, less than about 500 nM, less than about 250 nM, less than about 125 nM, less than about 60 nM, less than about 30 nM, less than about 10 nM, less than about 5 nM, less than about 2 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

In some embodiments, the antibody is a chimeric antibody. In some embodiments, the antibody is a humanized antibody. In some embodiments, the antibody is a human antibody.

In some embodiments, the antibody is an anti-PSMA, anti-PRLR, anti-MUC16, anti-HER2, anti-EGFRvIII, anti-MET, or anti-STEAP2 antibody. In some embodiments, the antibody or antigen-binding fragment is anti-PSMA. In some embodiments, the antibody or antigen-binding fragment is anti-MUC16. In some embodiments, the antibody or antigen-binding fragment is anti-HER2. In some embodiments, the antibody or antigen-binding fragment is anti-EGFRvIII. In some embodiments, the antibody or antigen-binding fragment is anti-MET. In some embodiments, the antibody or antigen-binding fragment is anti-PRLR or anti-STEAP2. In some embodiments, the antibody is an anti-PRLR or anti HER2 antibody. In some embodiments, the antibody or antigen-binding fragment thereof is anti-STEAP2. In some embodiments, the antibody or antigen-binding fragment thereof is anti-PRLR.

The antibody can have binding specificity for any antigen deemed suitable to those of skill in the art. In certain embodiments, the antigen is a transmembrane molecule (e.g., receptor). In one embodiment, the antigen is expressed on a tumor. In some embodiments, the binding agents interact with or bind to tumor antigens, including antigens specific for a type of tumor or antigens that are shared, overexpressed, or modified on a particular type of tumor. In one embodiment, the antigen is expressed on solid tumors. Exemplary antigens include, but are not limited to, lipoproteins; alpha1-antitrypsin; a cytotoxic T-lymphocyte associated antigen (CTLA), such as CTLA-4; vascular endothelial growth factor (VEGF); receptors for hormones or growth factors; protein A or D; fibroblast growth factor receptor 2 (FGFR2), EpCAM, GD3, FLT3, PSMA, PSCA, MUC1, MUC16, STEAP, STEAP2, CEA, TENB2, EphA receptors, EphB receptors, folate receptor, FOLRI, mesothelin, cripto, alphavbeta6, integrins, VEGF, VEGFR, EGFR, transferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80. CD81, CD103, CD105, CD134, CD137, CD138, CD152, or an antibody which binds to one or more tumor-associated antigens or cell-surface receptors disclosed in US Publication No. 2008/0171040 or US Publication No. 2008/0305044 each incorporated in their entirety by reference; erythropoietin; osteoinductive factors; immunotoxins; a bone morphogenetic protein (BMP); T-cell receptors; surface membrane proteins; integrins, such as CD11a, CD11b, CD11c, CD18, an ICAM, VLA-4 and VCAM; a tumor associated antigen such as AFP, ALK, B7H4, BAGE proteins, 0-catenin, brc-abl, BRCA1, BORIS, CA9 (carbonic anhydrase IX), caspase-8, CD20, CD40, CD123, CDK4, CEA, CLEC12A, c-kit, cMET, CTLA4, cyclin-B1, CYP1B1, EGFR, EGFRvIII, endoglin, Epcam, EphA2, ErbB2/Her2, ErbB3/Her3, ErbB4/Her4, ETV6-AML, Fra-1, FOLR1, GAGE proteins, GD2, GD3, GloboH, glypican-3, GM3, gp100, Her2, HLA/B-raf, HLA/EBNA1, HLA/k-ras, HLA/MAGE-A3, hTERT, IGF1R, LGR5, LMP2, MAGE proteins, MART-1, mesothelin, ML-IAP, Mucd, Muc16, CA-125, MUM1, NA17, NGEP, NY-BR1, NY-BR62, NY-BR85, NY-ESO1, OX40, p15, p53, PAP, PAX3, PAX5, PCTA-1, PDGFR-a, PDGFR-0, PDGF-A, PDGF-B, PDGF-C, PDGF-D, PLAC1, PRLR, PRAME, PSCA, PSGR, PSMA (FOLH1), RAGE proteins, Ras, RGS5, Rho, SART-1, SART-3, Steap-1, Steap-2, STn, survivin, TAG-72, TGF-β, TMPRSS2, Tn, TNFRSF17, TRP-1, TRP-2, tyrosinase, and uroplakin-3, and fragments of any of the above-listed polypeptides; cell-surface expressed antigens; MUC16; c-MET; molecules such as class A scavenger receptors including scavenger receptor A (SR-A), and other membrane proteins such as B7 family-related member including V-set and Ig domain-containing 4 (VSIG4), Colony stimulating factor 1 receptor (CSF1R), asialoglycoprotein receptor (ASGPR), and Amyloid beta precursor-like protein 2 (APLP-2). In some embodiments, the antigen is PRLR or HER2. In some embodiments, the antigen is STEAP2. In some embodiments the antigen is human STEAP2. In some examples, the MAGE proteins are selected from MAGE-1, -2, -3, -4, -6, and -12. In some examples, the GAGE proteins are selected from GAGE-1 and GAGE-2.

Exemplary antigens also include, but are not limited to, BCMA, SLAMF7, GPNMB, and UPK3A. Exemplary antigens also include, but are not limited to, MUC16, STEAP2, and HER2.

In some embodiments, the antigens include MUC16. In some embodiments, the antigens include STEAP2. In some embodiments, the antigens include PSMA. In some embodiments, the antigens include HER2. In some embodiments, the antigen is prolactin receptor (PRLR) or prostate-specific membrane antigen (PSMA). In some embodiments, the antigen is MUC16. In some embodiments, the antigens include PSMA. In some embodiments, the antigen is HER2. In some embodiments, the antigen is STEAP2.

In certain embodiments, the antibody comprises a glutamine residue at one or more heavy chain positions numbered 295 in the EU numbering system. In this disclosure, this position is referred to as glutamine 295, or as Gln295, or as Q295. Those of skill will recognize that this is a conserved glutamine residue in the wild type sequence of many antibodies. In other useful embodiments, the antibody can be engineered to comprise a glutamine residue. In certain embodiments, the antibody comprises one or more N297Q mutations. Techniques for modifying an antibody sequence to include a glutamine residue are within the skill of those in the art (see, e.g., Ausubel et al. Current Protoc. Mol. Biol.).

In some embodiments, the antibody, or antigen-binding fragment thereof, conjugated to the linker-payload or payload can be an antibody that targets STEAP2. Suitable anti-STEAP2 antibodies or antigen binding fragments thereof include those, for example, in International Publication No. WO 2018/058001 A1, including those comprising amino acid sequences disclosed in Table 1, on page 75 therein. In some embodiments, an anti-STEAP2 antibody is H1H7814N of WO 2018/058001 A1, comprising the CDRs of H1M7814N in the same publication. In some embodiments, an anti-STEAP2 antibody comprises a heavy chain complementarity determining region (HCDR)-1 comprising SEQ ID NO: 2; an HCDR2 comprising SEQ ID NO: 3; an HCDR3 comprising SEQ ID NO: 4; a light chain complementarity determining region (LCDR)-1 comprising SEQ ID NO: 6; an LCDR2 comprising SEQ ID NO: 7; and an LCDR3 comprising SEQ ID NO: 8. In some embodiments, an anti-STEAP2 antibody comprises a heavy chain variable region (HCVR) comprising SEQ ID NO: 1 and a light chain variable region (LCVR) comprising SEQ ID NO: 5. In any of the foregoing embodiments, the anti-STEAP2 antibody can be prepared by site-directed mutagenesis to insert a glutamine residue at a site without resulting in disabled antibody function or binding. For example, in any of the foregoing embodiments, the anti-STEAP2 antibody can comprise an Asn297Gln (N297Q) mutation. Such antibodies having an N297Q mutation can also contain one or more additional naturally occurring glutamine residues in their variable regions, which can be accessible to transglutaminase and therefore capable of conjugation to a payload or a linker-payload (Table A). In certain embodiments, the antibody or antigen-binding fragment thereof comprises three heavy chain complementarity determining regions (HCDR1, HCDR2, and HCDR3) within a heavy chain variable region (HCVR) amino acid sequence of SEQ ID NO: 1; and three light chain complementarity determining regions (LCDR1, LCDR2, and LCDR3) within a light chain variable region (LCVR) amino acid sequence of SEQ ID NO:5. In certain embodiments, the antibody or antigen-binding fragment thereof comprises an HCVR amino acid sequence of SEQ ID NO:1; and an LCVR amino acid sequence of SEQ ID NO:5. International Publication No. WO 2018/058001 A1 is hereby incorporated herein by reference in its entirety.

In some embodiments, the antibody, or antigen-binding fragment thereof, conjugated to the linker-payload or payload can be an antibody that targets human prolactin receptor (PRLR). Suitable anti-PRLR antibodies or antigen-binding fragments thereof include those, for example, in International Publication No. WO 2015/026907 A1, including those comprising amino acid sequences disclosed in Table 1, on page 36 therein. In some embodiments, an anti-PRLR antibody is H1H6958N2 of WO 2015/026907 A1, comprising the CDRs of H2M6958N2 in the same publication. In some embodiments, an anti-PRLR antibody comprises a heavy chain complementarity determining region (HCDR)-1 comprising SEQ ID NO: 10; an HCDR2 comprising SEQ ID NO: 11; an HCDR3 comprising SEQ ID NO: 12; a light chain complementarity determining region (LCDR)-1 comprising SEQ ID NO: 14; an LCDR2 comprising SEQ ID NO: 15; and an LCDR3 comprising SEQ ID NO: 16. In some embodiments, an anti-PRLR antibody comprises a heavy chain variable region (HCVR) comprising SEQ ID NO: 9 and a light chain variable region (LCVR) comprising SEQ ID NO: 13. In any of the foregoing embodiments, the anti-PRLR antibody can be prepared by site-directed mutagenesis to insert a glutamine residue at a site without resulting in disabled antibody function or binding. For example, in any of the foregoing embodiments, the anti-PRLR antibody can comprise an Asn297Gln (N297Q) mutation. Such antibodies having an N297Q mutation can also contain one or more additional naturally occurring glutamine residues in their variable regions, which can be accessible to transglutaminase and therefore capable of conjugation to a payload or a linker-payload (Table A). In certain embodiments, the antibody or antigen-binding fragment thereof comprises three heavy chain complementarity determining regions (HCDR1, HCDR2, and HCDR3) within a heavy chain variable region (HCVR) amino acid sequence of SEQ ID NO:9; and three light chain complementarity determining regions (LCDR1, LCDR2, and LCDR3) within a light chain variable region (LCVR) amino acid sequence of SEQ ID NO:13. In certain embodiments, the antibody or antigen-binding fragment thereof comprises an HCVR amino acid sequence of SEQ ID NO:9; and an LCVR amino acid sequence of SEQ ID NO:13. International Publication No. WO 2015/026907 A1 is hereby incorporated herein by reference in its entirety.

TABLE A
Sequences of Exemplary Antibodies H1H7814N (anti-STEAP2)
and H1H6958N2 (anti-PRLR)

| SEQ ID | Molecule / | | |
|---|---|---|---|
| NO: | Antibody | Region | Sequence |
| 1 | H1H7814N | HCVR | QVQLVESGGGVVQPGRSLRLSCVASGFTISSYGMNWVRQAPG |
| | | | KGLEWVAVISYDGGNKYSVDSVKGRFTISRDNSKNTLYLQMN |
| | | | SLRAEDSAVYYCARGRYFDLWGRGTLVTVSS |
| 2 | H1H7814N | HCDR1 | GFTISSYG |
| 3 | H1H7814N | HCDR2 | ISYDGGNK |
| 4 | H1H7814N | HCDR3 | ARGRYFDL |
| 5 | H1H7814N | LCVR | DIQMTQSPSTLSASVGDRVTITCRASQSISSWLAWYQQKPGR |
| | | | APNLLISKASSLKSGVPSRFSGSGSGTEFTLTVSSLQPDDFA |
| | | | TYYCQQYYSYSYTFGQGTKLEIK |
| 6 | H1H7814N | LCDR1 | QSISSW |
| 7 | H1H7814N | LCDR2 | KAS |
| 8 | H1H7814N | LCDR3 | QQYYSYSYT |
| 9 | H1H6958N2 | HCVR | QVQLVESGGGVVQPGRSLRLSCGASGFTFRNYGMQWVRQGPG |
| | | | KGLEWVTLISFDGNDKYYADSVKGRFTISRDNSKNTLFLQMN |
| | | | SLRTEDTAVYYCARGGDFDYWGQGTLVTVSS |
| 10 | H1H6958N2 | HCDR1 | GFTFRNYG |
| 11 | H1H6958N2 | HCDR2 | ISFDGNDK |
| 12 | H1H6958N2 | HCDR3 | ARGGDFDY |
| 13 | H1H6958N2 | LCVR | DIQMTQSPSSLSASVGDRVTITCRASQDIRKDLGWYQQKPGK |
| | | | APKRLIYAASSLHSGVPSRFSGSGSGTEFTLTISSLQPEDFA |
| | | | TYYCLQHNSYPMYTFGQGTKLEIK |
| 14 | H1H6958N2 | LCDR1 | QDIRKD |
| 15 | H1H6958N2 | LCDR2 | AAS |
| 16 | H1H6958N2 | LCDR3 | LQHNSYPMYT |
| 17 | hPRLR ecto- | | MHRPRRRGTRPPPLALLAALLLAARGADAQLPPGKPEIFKCR |
| | MMH | | SPNKETFTCWWRPGTDGGLPTNYSLTYHREGETLMHECPDYI |
| | | | TGGPNSCHFGKQYTSMWRTYIMMVNATNQMGSSFSDELYVDV |
| | | | TYIVQPDPPLELAVEVKQPEDRKPYLWIKWSPPTLIDLKTGW |
| | | | FTLLYEIRLKPEKAAEWEIHFAGQQTEFKILSLHPGQKYLVQ |
| | | | VRCKPDHGYWSAWSPATFIQIPSDFTMNDEQKLISEEDLGGE |
| | | | QKLISEEDLHHHHHH |

This disclosure provides antibodies or antigen-binding fragments thereof that specifically bind STEAP2, comprising an HCVR comprising an amino acid sequence selected from any of the HCVR amino acid sequences listed in Table A, or a substantially similar sequence thereof having at least 90%, at least 95%, at least 98%, or at least 99% sequence identity thereto.

This disclosure also provides antibodies or antigen-binding fragments thereof that specifically bind STEAP2, comprising an LCVR comprising an amino acid sequence selected from any of the LCVR amino acid sequences listed in Table A, or a substantially similar sequence thereof having at least 90%, at least 95%, at least 98%, or at least 99% sequence identity thereto.

This disclosure also provides antibodies or antigen-binding fragments thereof that specifically bind STEAP2, comprising an HCVR and an LCVR amino acid sequence pair (HCVR/LCVR) comprising any of the HCVR amino acid sequences listed in Table A paired with any of the LCVR amino acid sequences listed in Table A. According to certain embodiments, this disclosure provides antibodies, or antigen-binding fragments thereof, comprising an HCVR/LCVR amino acid sequence pair contained within any of the exemplary anti-STEAP2 antibodies listed in Table A. In certain embodiments, the HCVR/LCVR amino acid sequence pair is selected from the group consisting of: 250/258; as described in International Publication No. WO 2018/058001 A1, the contents of which are incorporated herein by reference in its entirety.

This disclosure also provides antibodies or antigen-binding fragments thereof that specifically bind STEAP2, comprising a heavy chain CDR1 (HCDR1) comprising an amino acid sequence selected from any of the HCDR1 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind STEAP2, comprising a heavy chain CDR2 (HCDR2) comprising an amino acid sequence selected from any of the HCDR2 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind STEAP2, comprising a heavy chain CDR3 (HCDR3) comprising an amino acid sequence selected from any of the HCDR3 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind STEAP2, comprising a light chain CDR1 (LCDR1) comprising an amino acid sequence selected from any of the LCDR1 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind STEAP2, comprising a light chain CDR2 (LCDR2) comprising an amino acid sequence selected from any of the LCDR2 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind STEAP2, comprising a light chain CDR3 (LCDR3) comprising an amino acid sequence selected from any of the LCDR3 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind STEAP2, comprising an HCDR3 and an LCDR3 amino acid sequence pair (HCDR3/LCDR3) comprising any of the HCDR3 amino acid sequences listed in Table A paired with any of the LCDR3 amino acid sequences listed in Table A. According to certain embodiments, this disclosure provides antibodies, or antigen-binding fragments thereof, comprising an HCDR3/LCDR3 amino acid sequence pair contained within any of the exemplary anti-STEAP2 antibodies listed in Table A. In certain embodiments, the HCDR3/LCDR3 amino acid sequence pair is selected from the group consisting of: 256/254; as described in International Publication No. WO 2018/058001 A1, the contents of which are incorporated herein by reference in its entirety.

This disclosure also provides antibodies or antigen-binding fragments thereof that specifically bind STEAP2, comprising a set of six CDRs (i.e., HCDR1-HCDR2-HCDR3-LCDR1-LCDR2-LCDR3) contained within any of the exemplary anti-STEAP2 antibodies listed in Table A. In certain embodiments, the HCDR1-HCDR2-HCDR3-LCDR1-LCDR2-LCDR3 amino acid sequence set is selected from the group consisting of: 252-254-256-260-262-264; as described in International Publication No. WO 2018/058001 A1, the contents of which are incorporated herein by reference in its entirety.

In a related embodiment, this disclosure provides antibodies, or antigen-binding fragments thereof that specifically bind STEAP2, comprising a set of six CDRs (i.e., HCDR1-HCDR2-HCDR3-LCDR1-LCDR2-LCDR3) contained within an HCVR/LCVR amino acid sequence pair as defined by any of the exemplary anti-STEAP2 antibodies listed in Table A. For example, this disclosure includes antibodies or antigen-binding fragments thereof that specifically bind STEAP2, comprising the HCDR1-HCDR2-HCDR3-LCDR1-LCDR2-LCDR3 amino acid sequence set contained within an HCVR/LCVR amino acid sequence pair selected from the group consisting of: 250/258; as described in International Publication No. WO 2018/058001 A1, the contents of which are incorporated herein by reference in its entirety. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); A1-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

This disclosure provides antibodies or antigen-binding fragments thereof that specifically bind PRLR, comprising an HCVR comprising an amino acid sequence selected from any of the HCVR amino acid sequences listed in Table A, or a substantially similar sequence thereof having at least 90%, at least 95%, at least 98%, or at least 99% sequence identity thereto.

This disclosure also provides antibodies or antigen-binding fragments thereof that specifically bind PRLR, comprising an LCVR comprising an amino acid sequence selected from any of the LCVR amino acid sequences listed in Table A, or a substantially similar sequence thereof having at least 90%, at least 95%, at least 98%, or at least 99% sequence identity thereto.

This disclosure also provides antibodies or antigen-binding fragments thereof that specifically bind PRLR, comprising an HCVR and an LCVR amino acid sequence pair (HCVR/LCVR) comprising any of the HCVR amino acid sequences listed in Table A paired with any of the LCVR amino acid sequences listed in Table A. According to certain embodiments, this disclosure provides antibodies, or antigen-binding fragments thereof, comprising an HCVR/LCVR amino acid sequence pair contained within any of the exemplary anti-PRLR antibodies listed in Table A. In certain embodiments, the HCVR/LCVR amino acid sequence pair is selected from the group consisting of: 18/26; 66/74; 274/282; 290/298; and 370/378; as described in International Publication No. WO 2015/026907 A1, the contents of which are incorporated herein by reference in its entirety.

This disclosure also provides antibodies or antigen-binding fragments thereof that specifically bind PRLR, comprising a heavy chain CDR1 (HCDR1) comprising an amino acid sequence selected from any of the HCDR1 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind PRLR, comprising a heavy chain CDR2 (HCDR2) comprising an amino acid sequence selected from any of the HCDR2 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind PRLR, comprising a heavy chain CDR3 (HCDR3) comprising an amino acid sequence selected from any of the HCDR3 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind PRLR, comprising a light chain CDR1 (LCDR1) comprising an amino acid sequence selected from any of the LCDR1 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind PRLR, comprising a light chain CDR2 (LCDR2) comprising an amino acid sequence selected from any of the LCDR2 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind PRLR, comprising a light chain CDR3 (LCDR3) comprising an amino acid sequence selected from any of the LCDR3 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind PRLR, comprising an HCDR3 and an LCDR3 amino acid sequence pair (HCDR3/LCDR3) comprising any of the HCDR3 amino acid sequences listed in Table A paired with any of the LCDR3 amino acid sequences listed in Table A. According to certain embodiments, this disclosure provides antibodies, or antigen-binding fragments thereof, comprising an HCDR3/LCDR3 amino acid sequence pair contained within any of the exemplary anti-PRLR antibodies listed in Table A. In certain embodiments, the HCDR3/LCDR3 amino acid sequence pair is selected from the group consisting of: 24/32; 72/80; 280/288; 296/304; and 376/384; as described in International Publication No. WO 2015/026907 A1, the contents of which are incorporated herein by reference in its entirety.

This disclosure also provides antibodies or antigen-binding fragments thereof that specifically bind PRLR, comprising a set of six CDRs (i.e., HCDR1-HCDR2-HCDR3-LCDR1-LCDR2-LCDR3) contained within any of the exemplary anti-PRLR antibodies listed in Table A. In certain embodiments, the HCDR1-HCDR2-HCDR3-LCDR1-LCDR2-LCDR3 amino acid sequence set is selected from the group consisting of: 20-22-24-28-30-32; 68-70-72-76-78-80; 276-278-280-284-286-288; 292-294-296-300-302-304; and 372-374-376-380-382-384; as described in International Publication No. WO 2015/026907 A1, the contents of which are incorporated herein by reference in its entirety.

In a related embodiment, this disclosure provides antibodies, or antigen-binding fragments thereof that specifically bind PRLR, comprising a set of six CDRs (i.e., HCDR1-HCDR2-HCDR3-LCDR1-LCDR2-LCDR3) contained within an HCVR/LCVR amino acid sequence pair as defined by any of the exemplary anti-PRLR antibodies listed in Table A. For example, this disclosure includes antibodies or antigen-binding fragments thereof that specifically bind PRLR, comprising the HCDR1-HCDR2-HCDR3-LCDR1-LCDR2-LCDR3 amino acid sequence set contained within an HCVR/LCVR amino acid sequence pair selected from the group consisting of: 18/26; 66/74; 274/282; 290/298; and 370/378; as described in International Publication No. WO 2015/026907 A1, the contents of which are incorporated herein by reference in its entirety. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); A1-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

The binding agent linkers can be bonded to the binding agent, e.g., antibody or antigen-binding molecule, through an attachment at a particular amino acid within the antibody or antigen-binding molecule. Exemplary amino acid attachments that can be used in the context of this embodiment of the disclosure include, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; US 2013/0101546; and US 2012/0585592), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenocysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Linkers can also be conjugated to an antigen-binding protein via attachment to carbohydrates (see, e.g., US 2008/0305497, WO 2014/065661, and Ryan et al., Food & Agriculture Immunol., 2001, 13:127-130).

In some examples, the binding agent is an antibody or antigen binding molecule, and the antibody is bonded to the linker through a lysine residue. In some embodiments, the antibody or antigen binding molecule is bonded to the linker through a cysteine residue.

Linkers can also be conjugated to one or more glutamine residues via transglutaminase-based chemo-enzymatic conjugation (see, e.g., Dennler et al., Bioconjugate Chem. 2014, 25, 569-578). For example, in the presence of transglutaminase, one or more glutamine residues of an antibody can be coupled to a primary amine compound. Primary amine compounds include, e.g., payloads or linker-payloads, which directly provide transglutaminase-modified antibody drug conjugates via transglutaminase-mediated coupling. Primary amine compounds also include linkers and spacers that are functionalized with reactive groups that can be subsequently reacted with further compounds towards the synthesis of antibody drug conjugates (e.g., in certain embodiments, transglutaminase-modified antibody drug conjugates). Antibodies comprising glutamine residues can be isolated from natural sources or engineered to comprise one or more glutamine residues. Techniques for engineering glutamine residues into an antibody polypeptide chain (glutaminyl-modified antibodies or antigen binding molecules) are within the skill of the practitioners in the art. In certain embodiments, the antibody is aglycosylated.

In certain embodiments, the antibody, glutaminyl-modified antibody, or transglutaminase-modified antibody or antigen binding fragments thereof comprise at least one glutamine residue in at least one polypeptide chain sequence. In certain embodiments, the antibody, glutaminyl-modified antibody, or transglutaminase-modified antibody or antigen binding fragments thereof comprise two heavy chain polypeptides, each with one Gln295 or Q295 residue. In further embodiments, the antibody, glutaminyl-modified antibody, or transglutaminase-modified antibody or antigen binding fragments thereof comprise one or more glutamine residues at a site other than a heavy chain 295. Included herein are antibodies of this section bearing N297Q mutation(s) described herein.

### Primary Amine Compounds

In certain embodiments, primary amine compounds useful for the transglutaminase-mediated coupling of an antibody (or antigen binding compound) comprising one or more glutamine residues (i.e., resulting in a transglutaminase-modified antibody or antigen binding fragment thereof) can be any primary amine compound deemed useful by the practitioner of ordinary skill. Generally, the primary amine compound has the formula H₂N—R, where R can be any group compatible with the antibody and reaction conditions. In certain embodiments, R is alkyl, substituted alkyl, heteroalkyl, or substituted heteroalkyl.

In some embodiments, the primary amine compound comprises a reactive group or protected reactive group. Useful reactive groups include azides, alkynes, cycloalkynes, thiols, alcohols, ketones, aldehydes, carboxylic acids, esters, amides, hydrazides, anilines, and amines. In certain embodiments, the reactive group is selected from the group consisting of azide, alkyne, sulfhydryl, cycloalkyne, aldehyde, and carboxyl.

In certain embodiments, the primary amine compound is according to the formula H₂N-LL-X, where LL is a divalent spacer and X is a reactive group or protected reactive group. In particular embodiments, LL is a divalent polyethylene glycol (PEG) group. In certain embodiments, X is selected from the group consisting of —SH, —N₃, alkyne, aldehyde, and tetrazole. In particular embodiments, X is —N₃.

In certain embodiments, the primary amine compound is according to one of the following formulas:

H₂N—(CH₂)ₙ—X;

H₂N—(CH₂CH₂O)ₙ—(CH₂)ₚ—X;

H₂N—(CH₂)ₙ—N(H)C(O)—(CH₂)ₘ—X;

H₂N—(CH₂CH₂O)ₙ—N(H)C(O)—(CH₂CH₂O)ₘ—(CH₂)ₚ—X;

H₂N—(CH₂)ₙ—C(O)N(H)—(CH₂)ₘ—X;

H₂N—(CH₂CH₂O)ₙ—C(O)N(H)—(CH₂CH₂O)ₘ—(CH₂)ₚ—X;

H₂N—(CH₂)ₙ—N(H)C(O)—(CH₂CH₂O)ₘ—(CH₂)ₚ—X;

H₂N—(CH₂CH₂O)ₙ—N(H)C(O)—(CH₂)ₘ—X;

H₂N—(CH₂)ₙ—C(O)N(H)—(CH₂CH₂O)ₘ—(CH₂)ₚ—X; and

H₂N—(CH₂CH₂O)ₙ—C(O)N(H)—(CH₂)ₘ—X;

where n is an integer selected from 1 to 12;
m is an integer selected from 0 to 12;
p is an integer selected from 0 to 2;
and X is selected from the group consisting of —SH, —N₃, —C≡CH, —C(O)H, tetrazole, and any of

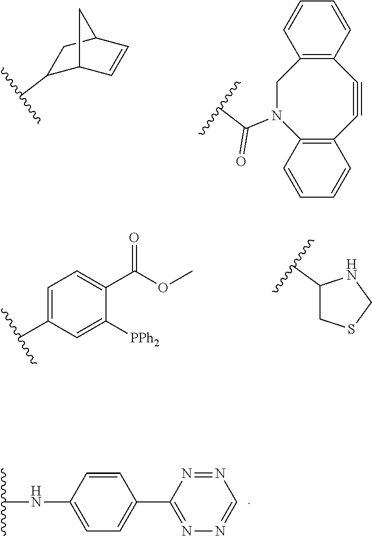

In the above, any of the alkyl or alkylene (i.e., —CH₂—) groups can optionally be substituted, for example, with C₁₋₈ alkyl, methylformyl, or —SO₃H. In certain embodiments, the alkyl groups are unsubstituted.

In certain embodiments, the primary amine compound is selected from the group consisting of:

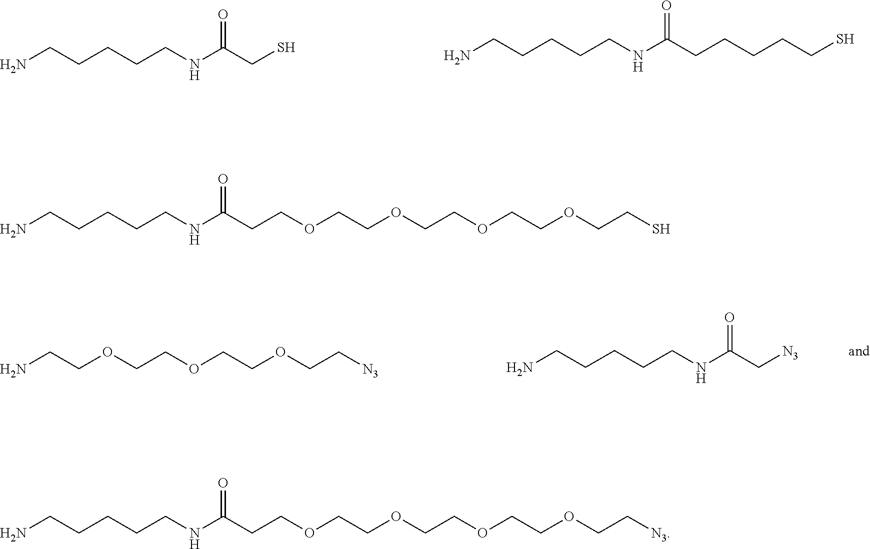

In particular embodiments, the primary amine compound is

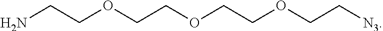 [SMILES: [N-]=[N+]=NCCOCCOCCOCCN]

Exemplary conditions for the above reactions are provided in the Examples below.

### Linkers

In certain embodiments, the linker L portion of the conjugates described herein is a moiety, for instance a divalent moiety, that covalently links a binding agent to a payload compound described herein. In other instances, the linker L is a trivalent or multivalent moiety that covalently links a binding agent to a payload compound described herein. Suitable linkers may be found, for example, in Antibody-Drug Conjugates and Immunotoxins; Phillips, G. L., Ed.; Springer Verlag: New York, 2013; Antibody-Drug Conjugates; Ducry, L., Ed.; Humana Press, 2013; Antibody-Drug Conjugates; Wang, J., Shen, W.-C., and Zaro, J. L., Eds.; Springer International Publishing, 2015, the contents of each incorporated herein in their entirety by reference. In certain embodiments, the linker L portion of the linker-payloads or linker-prodrug payloads described herein is a moiety covalently linked to a payload or prodrug payload compound described herein, capable of divalently and covalently linking a binding agent to a payload or prodrug payload compound described herein. In other instances, the linker L portion of the linker-payloads described herein is a moiety covalently linked to a payload or prodrug payload compound described herein, capable of covalently linking, as a trivalent or multivalent moiety, a binding agent to a payload or prodrug payload compound described herein. Payload or prodrug payload compounds include compounds of Formulae I, Ia, Iaa, II, III, IV, V, and VI above, and their residues following bonding or incorporation with linker L are linker-payloads or linker-prodrug payloads. The linker-payloads can be further bonded to binding agents such as antibodies or antigen binding fragments thereof to form antibody-drug conjugates. Those of skill in the art will recognize that certain functional groups of payload moieties are convenient for linking to linkers and/or binding agents. For example, in certain embodiments, the linker is absent and payloads or prodrug payloads are directly bonded to binding agents. In one embodiment, payloads or prodrug payloads include terminal alkynes and binding agents include azides, where each alkyne and azide participate in regioisomeric click chemistry to bind payload or prodrug payload residues directly to binding agent residues. In another embodiment, payloads or prodrug payloads include carboxylic acids and binding agents include lysines, where each carboxylic acid and lysine participate in amide bond formation to bind payload or prodrug payload residues directly to binding agent residues. Payload functional groups further include amines (e.g., Formulae C, D, E, LPc, LPd, and LPe), quaternary ammonium ions (e.g., Formulae A and LPa), hydroxyls (e.g., Formulae C, D, E, LPc, LPd, and LPe), phosphates, carboxylic acids (e.g., in the form of esters upon linking to L, as in Formulae B, D, LPb, and LPd), hydrazides (e.g., Formulae B and LPb), amides (e.g., derived from anilines of Formula C and LPc, or amines of Formulae D, E, LPd, and LPe), and sugars.

In certain embodiments, the linkers are stable in physiological conditions. In certain embodiments, the linkers are cleavable, for instance, able to release at least the payload portion in the presence of an enzyme or at a particular pH range or value. In some embodiments, a linker comprises an enzyme-cleavable moiety. Illustrative enzyme-cleavable moieties include, but are not limited to, peptide bonds (i.e., distinguished from prodrug payloads having peptide bonds, as described elsewhere herein), ester linkages, hydrazones, β-glucuronide linkages, and disulfide linkages. In some embodiments, the linker comprises a cathepsin-cleavable linker. In some embodiments, the linker comprises a β-glucuronidase (GUSB)-cleavable linker (see, e.g., GUSB linkers from Creative Biolabs, creative-biolabs.com/adc/beta-glucuronide-linker.htm, or ACS Med. Chem. Lett. 2010, 1: 277-280).

In some embodiments, the linker comprises a non-cleavable moiety. In some embodiments, the non-cleavable linker is derived from

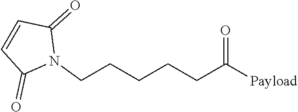

or a residue thereof. In some embodiments, the non-cleavable linker-payload residue

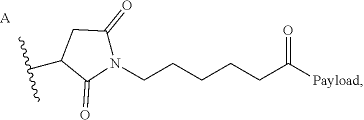

or a regioisomer thereof. In some embodiments, the non-cleavable linker is derived from

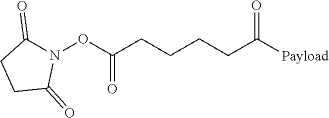

or a residue thereof. In some embodiments, the non-cleavable linker-payload residue is

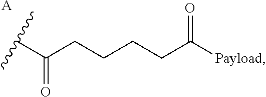

or a regioisomer thereof. In one embodiment, the linker is maleimide cyclohexane carboxylate or 4-(N-maleimidomethyl)cyclohexanecarboxylic acid (MCC). In the structures, [symbol] indicates a bond to a binding agent. In the structures, in some examples, [symbol] indicates a click chemistry residue which results from the reaction of, for example, a binding agent having an azide or alkyne functionality and a linker-payload having a complementary alkyne or azide functionality. In the structures, in other examples, [symbol] indicates a divalent sulfide which results from the reaction of, for example, one or more binding agent cysteines with one or more linkers or linker-payloads having maleimide functionality via Michael addition reactions. In the structures, in other examples, [symbol] indicates an amide bond which results from the reaction of, for example, one or more binding agent lysines with one or more linkers or linker-payloads having activated or unactivated carboxyl functionality, as would be appreciated by a person of skill in the art. In one embodiment, [symbol] indicates an amide bond which results from the reaction of, for example, one or more binding agent lysines with one or more linkers or linker-payloads having activated carboxyl functionality, as would be appreciated by a person of skill in the art.

In some embodiments, suitable linkers include, but are not limited to, those that are chemically bonded to two cysteine residues of a single binding agent, e.g., antibody. Such linkers can serve to mimic the antibody's disulfide bonds that are disrupted as a result of the conjugation process.

In some embodiments, the linker comprises one or more amino acids (i.e., distinguished from prodrug payloads comprising peptide bonds derived from distinguishable amino acids, as described elsewhere herein). Suitable amino acids include natural, non-natural, standard, non-standard, proteinogenic, non-proteinogenic, and L- or D- a-amino acids. In some embodiments, the linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or any combination thereof (e.g., dipeptides, tripeptides, oligopeptides, polypeptides, and the like). In certain embodiments, one or more side chains of the amino acids are linked to a side chain group, described below. In some embodiments, the linker is a peptide comprising or consisting of the amino acids valine and citrulline (e.g., divalent -Val-Cit- or divalent -VCit-). In some embodiments, the linker is a peptide comprising or consisting of the amino acids alanine and alanine, or divalent -AA-. In some embodiments, the linker is a peptide comprising or consisting of the amino acids glutamic acid and alanine, or -EA-. In some embodiments, the linker is a peptide comprising or consisting of the amino acids glutamic acid and glycine, or -EG-. In some embodiments, the linker is a peptide comprising or consisting of the amino acids glycine and glycine, or -GG-. In some embodiments, the linker is a peptide comprising or consisting of the amino acids glutamine, valine, and citrulline, or -Q-V-Cit- or -QVCit-. In some embodiments, the linker is a peptide comprising or consisting of the amino acids glutamic acid, valine, and citrulline, or -E-V-Cit- or -EVCit-. In some embodiments, the linker is a peptide comprising or consisting of the amino acids -GGGGS- (SEQ ID NO: 18). In some embodiments, the linker is a peptide comprising or consisting of the amino acids -GGGGG- (SEQ ID NO: 19). In some embodiments, the linker is a peptide comprising or consisting of the amino acids -GGGGK- (SEQ ID NO: 20). In some embodiments, the linker is a peptide comprising or consisting of the amino acids -GFGG- (SEQ ID NO: 21). In some embodiments, the linker is a peptide comprising or consisting of the amino acids -GG-. In some embodiments, the linker is a peptide comprising or consisting of the amino acids -GGG-. In some embodiments, the linker is a peptide comprising or consisting of the amino acids -GGGG- (SEQ ID NO: 22). In some embodiments, the linker is a peptide comprising or consisting of the amino acids -GGFG- (SEQ ID NO: 23). In some embodiments, the linker is a peptide comprising or consisting of the amino acids lysine, valine, and citrulline, or -KVCit-. In some embodiments, the linker is a peptide comprising or consisting of the amino acids -KVA-. In some embodiments, the linker is a peptide comprising or consisting of the amino acids -VA-. In any of the embodiments in this paragraph, and throughout this disclosure, the standard three-letter or one-letter amino acid designations are used, as would be appreciated by a person of skill in the art. Exemplary single-letter amino acid designations include, G for glycine, K for lysine, S for serine, V for valine, A for alanine, and F for phenylalanine.

In some embodiments, the linker comprises a self-immolative group. The self-immolative group can be any such group known to those of skill. In particular embodiments, the self-immolative group is p-aminobenzyl (PAB), or a derivative thereof. Useful derivatives include p-aminobenzyloxycarbonyl (PABC). Those of skill will recognize that a self-immolative group is capable of carrying out a chemical reaction which releases the remaining atoms of a linker from a payload.

In some embodiments the linker is:

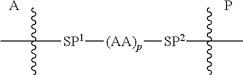

wherein:
SP¹ is a spacer;
SP² is a spacer;
[symbol] is one or more bonds to the binding agent;
[symbol] is one or more bonds to the payload;
each AA is an amino acid residue; and
p is an integer from zero to ten.
   In certain embodiments, each AA here within the linker L can be characterized as a second amino acid residue, in contrast to a first amino acid residue within a payload or prodrug payload, as described elsewhere herein. As would be appreciated by a person of skill in the art, in certain embodiments, more than one AA here within the linker L can be characterized as a second peptide residue, in contrast to a first peptide residue within a payload or prodrug payload, as described elsewhere herein.

The SP1 spacer is a moiety that connects the (AA)ₚ moiety or residue to the binding agent (BA) or to a reactive group residue which is bonded to BA. Suitable SP¹ spacers include, but are not limited to, those comprising alkylene or polyether, or both. The ends of the spacers, for example, the portion of the spacer bonded to the BA or an AA, can be moieties derived from reactive moieties that are used for purposes of coupling the antibody or an AA to the spacer during chemical synthesis of the conjugate. In certain embodiments, p is zero, one, two, three, or four. In particular embodiments, p is 2. In particular embodiments, p is 3. In particular embodiments, p is 4.

In some embodiments, the SP¹ spacer comprises an alkylene. In some embodiments, the SP¹ spacer comprises a C₅₋₇ alkylene. In some embodiments, the SP¹ spacer comprises a polyether. In some embodiments, the SP¹ spacer comprises a polymer of ethylene oxide such as polyethylene glycol.

In some embodiments, the SP¹ spacer is:

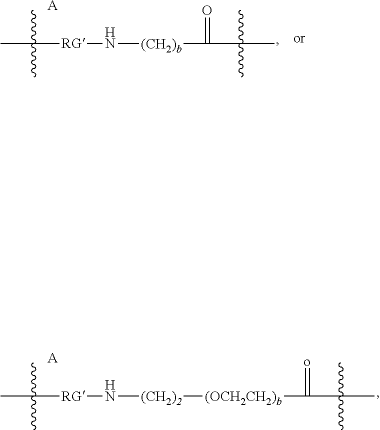

wherein:
RG′ is a reactive group residue following reaction of a reactive group RG with a binding agent;
[symbol] is a bond to the binding agent;
[symbol] is a bond to (AA)p where p is an integer from zero to ten; and
b is an integer from two to eight.

The reactive group RG can be any reactive group known to those of skill in the art to be capable of forming one or more bonds to the binding agent. The reactive group RG is a moiety comprising a portion in its structure that is capable of reacting with the binding agent (e.g., reacting with an antibody at its cysteine or lysine residues, or at an azide moiety, for example, a PEG-N₃ functionalized antibody at one or more glutamine residues) to form a compound of Formula A, A′, B, B′, C, C′, D, D′, E, or E′. Following conjugation to the binding agent, the reactive group becomes the reactive group residue (RG′). Illustrative reactive groups include, but are not limited to, those that comprise haloacetyl, isothiocyanate, succinimide, N-hydroxysuccinimide, or maleimide portions that are capable of reacting with the binding agent.

In certain embodiments, reactive groups include, but are not limited to, alkynes. In certain embodiments, the alkynes are alkynes capable of undergoing 1,3-cycloaddition reactions with azides in the absence of copper catalysts, such as strained alkynes. Strained alkynes are suitable for strain-promoted alkyne-azide cycloadditions (SPAAC), and include cycloalkynes, for example, cyclooctynes and benzannulated alkynes. Suitable alkynes include, but are not limited to, dibenzoazacyclooctyne or

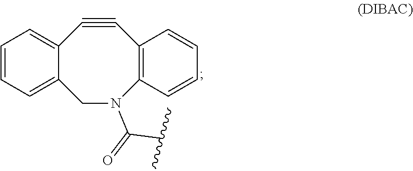 [SMILES: *C(=O)N1Cc2ccccc2C#Cc2ccccc21]

dibenzocyclooctyne or

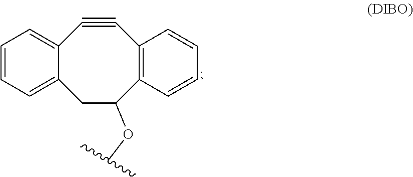 [SMILES: *OC1Cc2ccccc2C#Cc2ccccc21]

biarylazacyclooctynone or

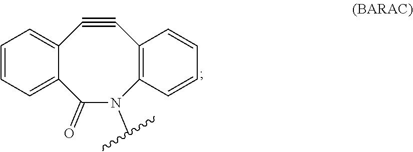 [SMILES: *N1C(=O)c2ccccc2C#Cc2ccccc21]

difluorinated cyclooctyne or

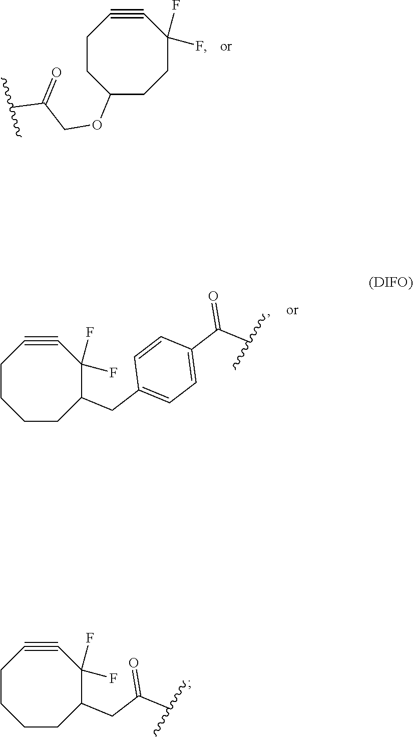 [SMILES: *C(=O)COC1CCC#CC(F)(F)CC1 ; *C(=O)c1ccc(CC2CCCCC#CC2(F)F)cc1 ; *C(=O)CC1CCCCC#CC1(F)F]

substituted, for example, fluorinated alkynes, aza-cycloalkynes, bicycle[6.1.0]nonyne or

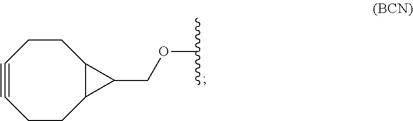 [SMILES: *OCC1C2CCC#CCCC21]

and derivatives thereof. Particularly useful alkynes include

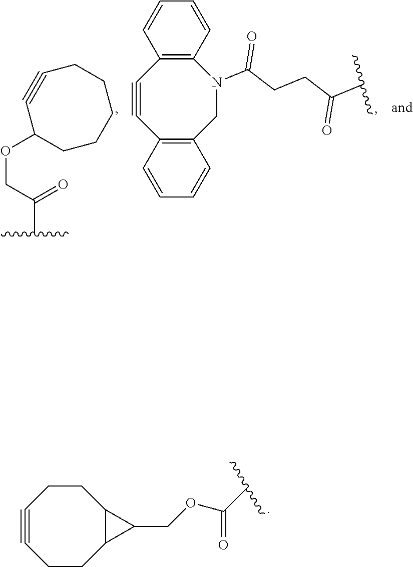

In certain embodiments, the binding agent is bonded directly to RG′. In certain embodiments, the binding agent is bonded to RG′ via a spacer, for instance SP⁴, located between [symbol] and RG′. In particular embodiments, the binding agent is bonded indirectly to RG′ via SP⁴, for example, a PEG spacer. As discussed in detail below, in certain embodiments, the binding agent is prepared by functionalizing with one or more azido groups. Each azido group is capable of reacting with RG to form RG′. In particular embodiments, the binding agent is derivatized with -PEG-N₃ linked to a glutamine residue (e.g., a transglutaminse-modified binding agent). Exemplary —N₃ derivatized binding agents, methods for their preparation, and methods for their use in reacting with RG are provided herein. In certain embodiments, RG is an alkyne suitable for participation in 1,3-cycloadditions, and RG′ is a regioisomeric 1,2,3-triazolyl moiety formed from the reaction of RG with an azido-functionalized binding agent. By way of further example, in certain embodiments, RG′ is linked to the binding agent as shown in

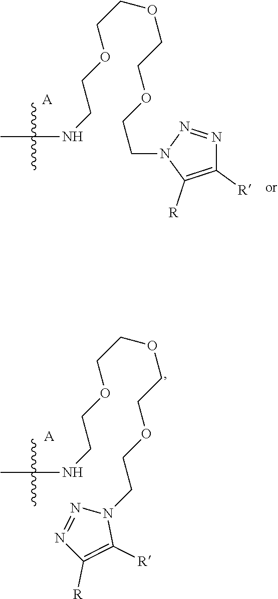

or a mixture of each regioisomer. Each R and R′ is as described or exemplified herein.

The SP² spacer, when present, is a moiety that connects the (AA)ₚ moiety to the payload. Suitable spacers include, but are not limited to, those described above as SP¹ spacers. Further suitable SP² spacers include, but are not limited to, those comprising alkylene or polyether, or both. The ends of the SP² spacers, for example, the portion of the spacer directly bonded to the payload, prodrug payload, or an AA, can be moieties derived from reactive moieties that are used for purposes of coupling the payload, prodrug payload, or AA to the SP² spacer during the chemical synthesis of the conjugate. In some examples, the ends of the SP² spacers, for example, the portion of the SP² spacer directly bonded to the payload, prodrug payload, or an AA, can be residues of reactive moieties that are used for purposes of coupling the payload, prodrug payload, or an AA to the spacer during the chemical synthesis of the conjugate.

In some embodiments, the SP² spacer, when present, is selected from the group consisting of —NH-(p-C₆H₄)—CH₂—, —NH-(p-C₆H₄)—CH₂OC(O)—, an amino acid, a dipeptide, a tripeptide, an oligopeptide, —O—, —N(H)—,

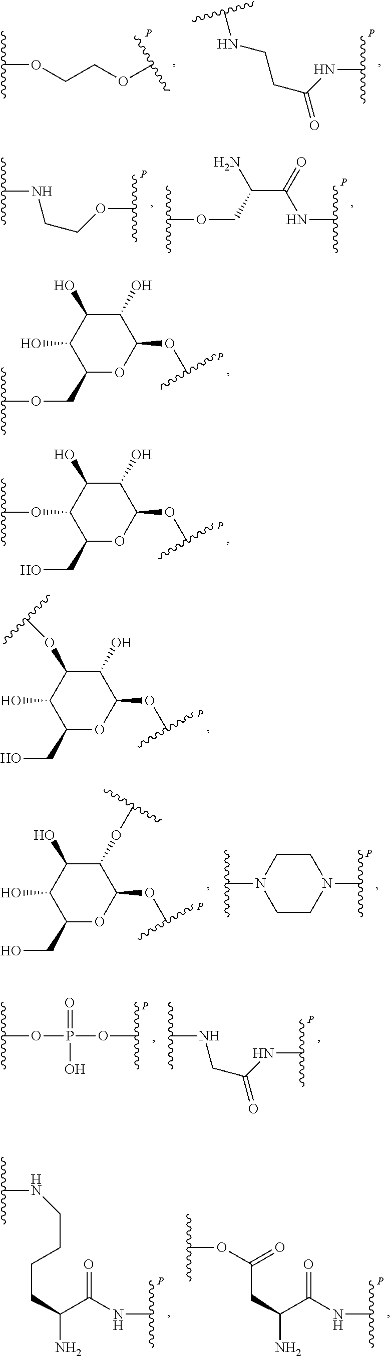

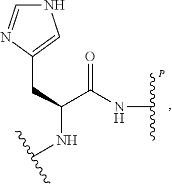

and any combinations thereof. In certain embodiments, each [symbol] is a bond to the payload or prodrug payload, and each [symbol] is a bond to (AA)ₚ.

In the above formulas, each (AA)ₚ is an amino acid or, optionally, a p-aminobenzyloxycarbonyl residue (PABC),

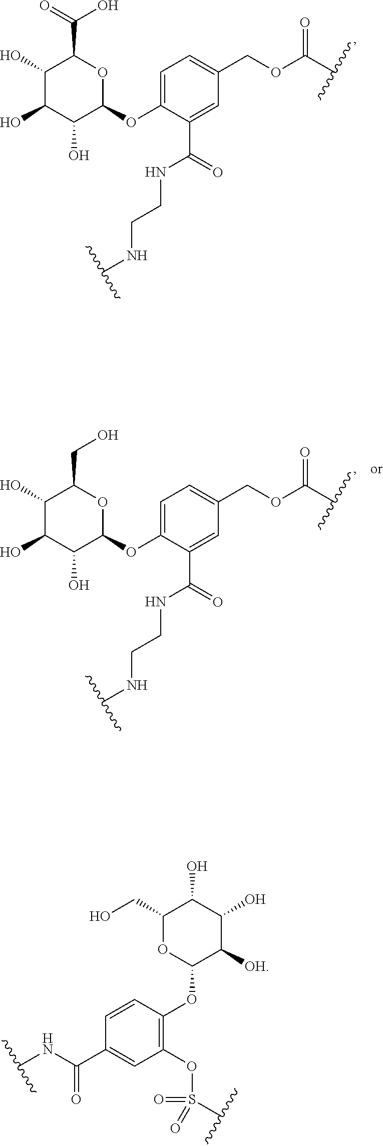 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O ; *NC(=O)c1ccc(O[C@@H]2O[C@H](CO)[C@H](O)[C@H](O)[C@H]2O)c(OS(*)(=O)=O)c1]

If PABC is present, then in particular embodiments only one PABC is present. In certain embodiments, the PABC residue, if present, is bonded to a terminal AA in the (AA)ₚ group, proximal to the payload or prodrug payload. If

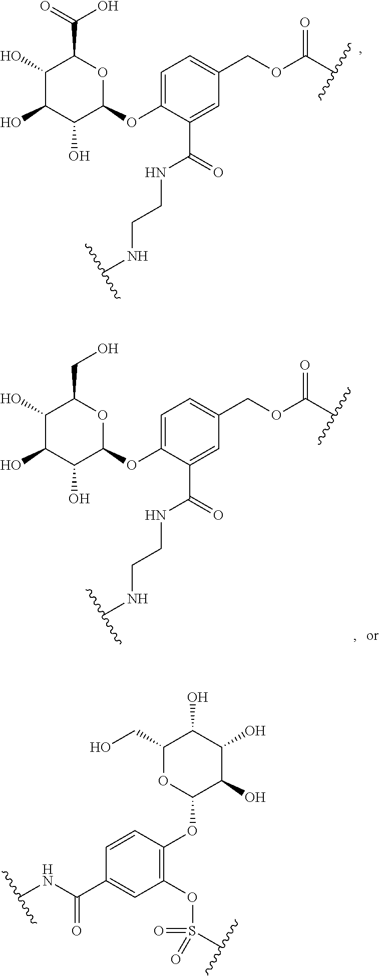 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O ; *NC(=O)c1ccc(O[C@@H]2O[C@H](CO)[C@H](O)[C@H](O)[C@H]2O)c(OS(*)(=O)=O)c1]

is present, then only

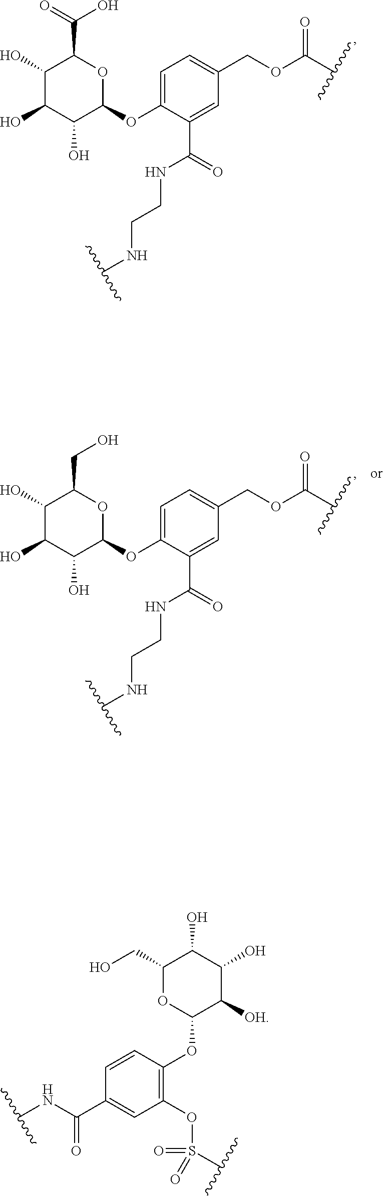 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O ; *NC(=O)c1ccc(O[C@@H]2O[C@H](CO)[C@H](O)[C@H](O)[C@H]2O)c(OS(*)(=O)=O)c1]

is present. In certain embodiments, the

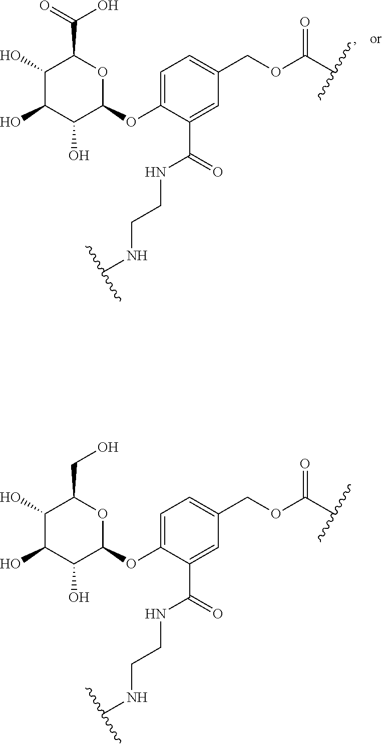 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O]

residue, if present, is bonded to the payload or prodrug payload via the benzyloxycarbonyl moiety, and no AA is present. In certain embodiments, the

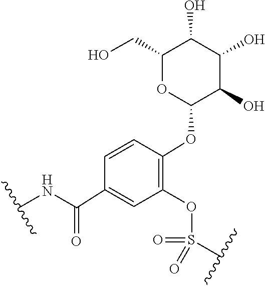 [SMILES: *NC(=O)c1ccc(O[C@@H]2O[C@H](CO)[C@H](O)[C@H](O)[C@H]2O)c(OS(*)(=O)=O)c1]

residue, if present, is bonded to the payload or prodrug payload via —O—. Suitable amino acids for each AA include natural, non-natural, standard, non-standard, proteinogenic, non-proteinogenic, and L- or D-α-amino acids. In some embodiments, the AA comprises alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or any combinations thereof (e.g., dipeptides, tripeptides, and oligopeptides, and the like). In certain embodiments, one or more side chains of the amino acids is linked to a side chain group, described below. In some embodiments, p is two. In some embodiments, the (AA)ₚ is valine-citrulline. In some embodiments, (AA)ₚ is citrulline-valine. In some embodiments, (AA)ₚ is valine-alanine. In some embodiments, (AA)ₚ is alanine-valine. In some embodiments, (AA)ₚ is valine-glycine. In some embodiments, (AA)ₚ is glycine-valine. In some embodiments, p is three. In some embodiments, the (AA)ₚ is valine-citrulline-PABC. In some embodiments, (AA)ₚ is citrulline-valine-PABC. In some embodiments, (AA)ₚ is glutamate-valine-citrulline. In some embodiments, (AA)ₚ is glutamine-valine-citrulline. In some embodiments, (AA)ₚ is lysine-valine-alanine. In some embodiments, (AA)ₚ is lysine-valine-citrulline. In some embodiments, p is four. In some embodiments, (AA)ₚ is glutamate-valine-citrulline-PAB. In some embodiments, (AA)ₚ is glutamine-valine-citrulline-PABC. Those of skill will recognize PABC as a residue of p-aminobenzyloxycarbonyl with the following structure:

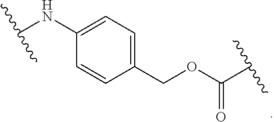 [SMILES: *Nc1ccc(COC(*)=O)cc1]

The PABC residue has been shown to facilitate cleavage of certain linkers in vitro and in vivo. Those of skill will recognize PAB as a divalent residue of p-aminobenzyl or —NH-(p-C₆H₄)—CH₂—.

In some embodiments, the linker is:

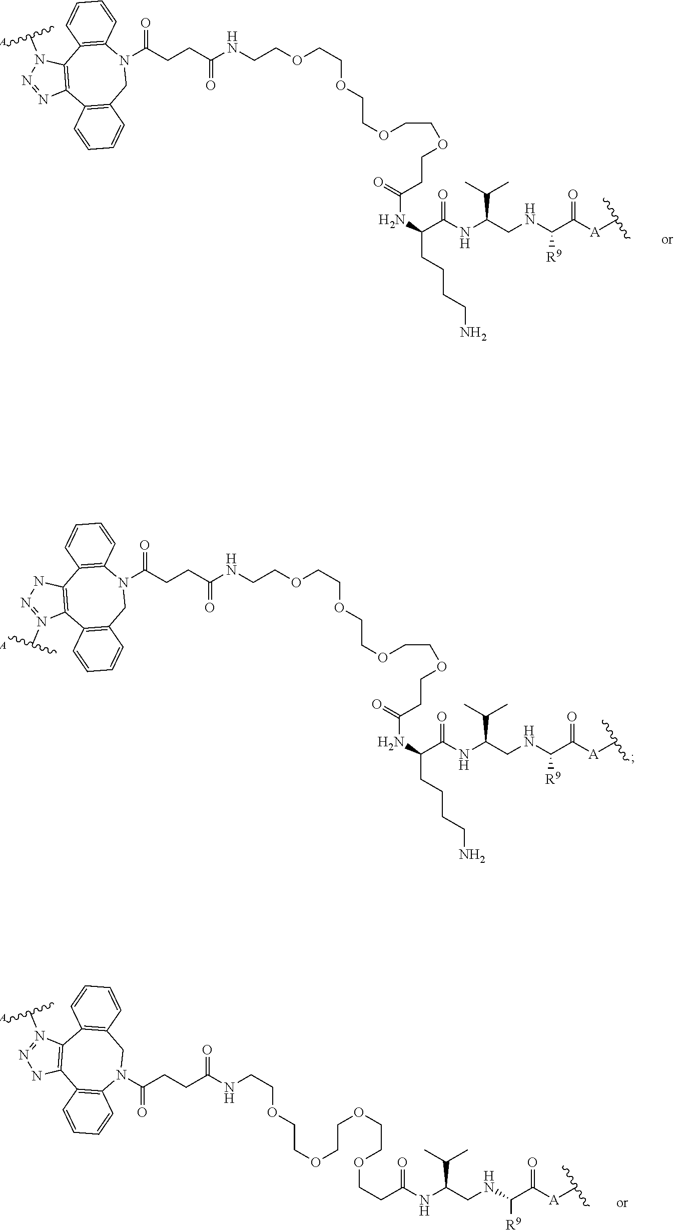

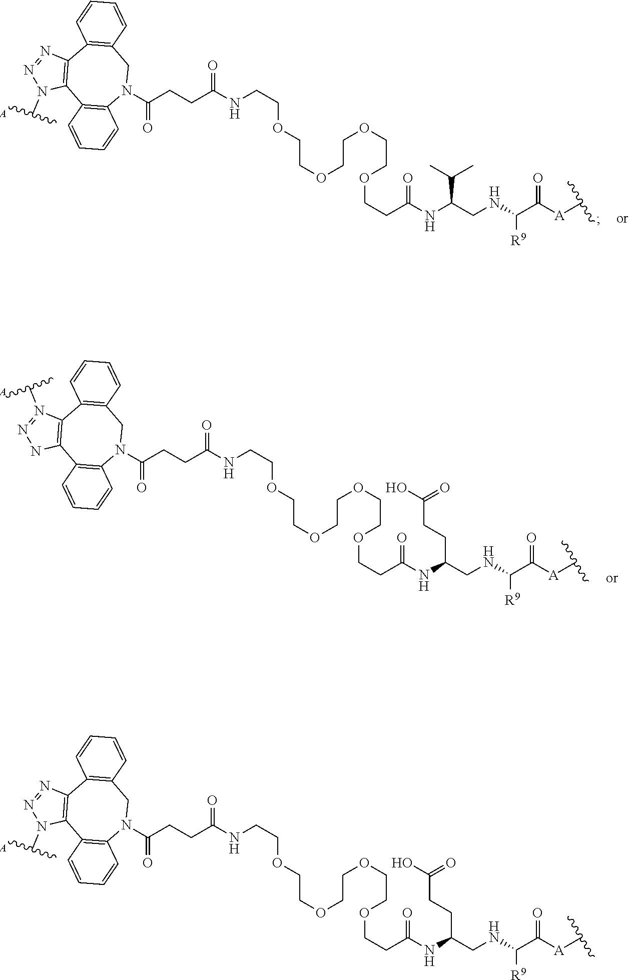

wherein
each [symbol] is a bond to a transglutaminase-modified binding agent;
each [symbol] is a bond to the payload;
each R⁹ is —CH₃ or —(CH₂)₃N(H)C(O)NH₂; and
each A is —O—, —NH—,

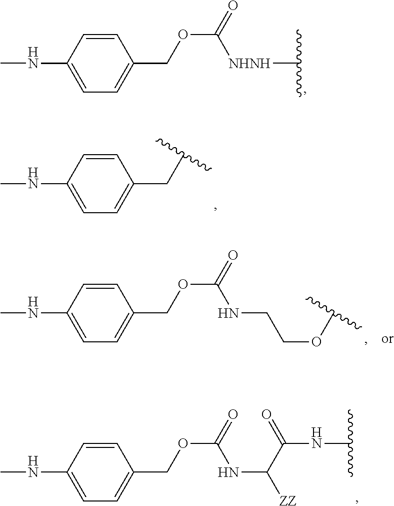

where ZZ is hydrogen, or a side chain for an amino acid as discussed elsewhere herein. By way of further example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

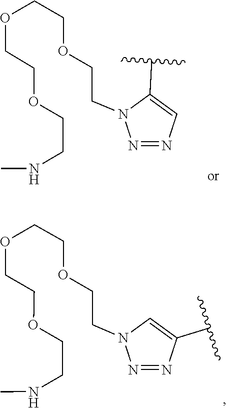 [SMILES: *c1cnnn1CCOCCOCCOCCNC ; *c1cn(CCOCCOCCOCCNC)nn1]

or a mixture thereof. Alternatively, in another embodiment, A is

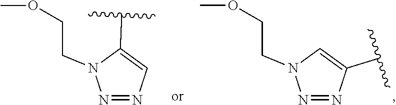

or a mixture thereof. In another embodiment, A is

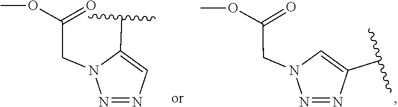 [SMILES: *c1cnnn1CC(=O)OC ; *c1cn(CC(=O)OC)nn1]

or a mixture thereof. In another embodiment, A is

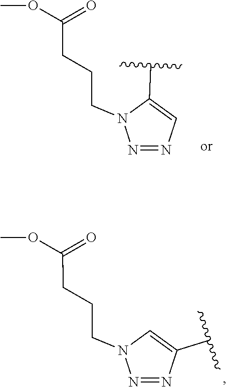 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent.

In some embodiments, the linker is:

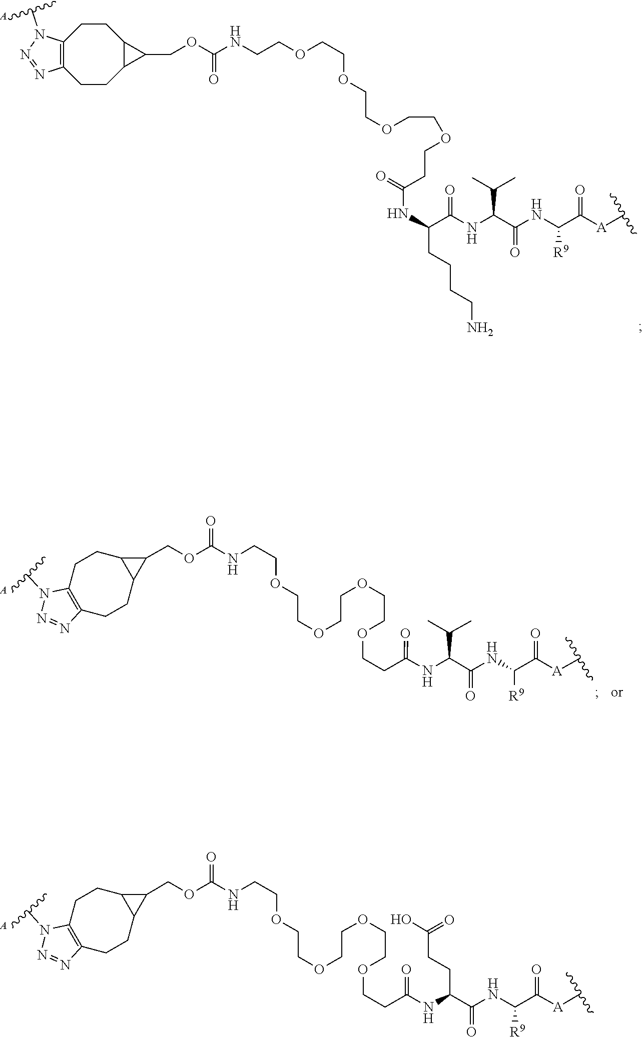

wherein:
each [symbol] is a bond to a transglutaminse-modified binding agent;
each [symbol] is a bond to the payload;
each R⁹ is —CH₃ or —(CH₂)₃N(H)C(O)NH₂; and
each A is —O—, —N(H)—,

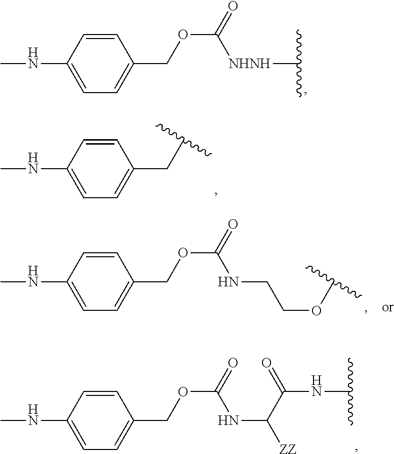

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

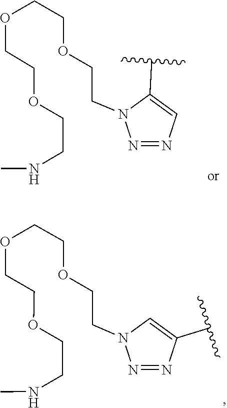 [SMILES: *c1cnnn1CCOCCOCCOCCNC ; *c1cn(CCOCCOCCOCCNC)nn1]

or a mixture thereof. Alternatively, in another embodiment, A is

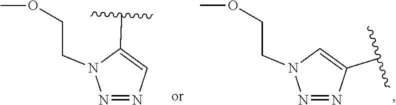

or a mixture thereof. In another embodiment, A is

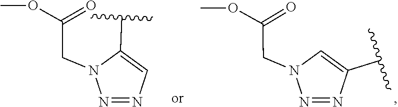 [SMILES: *c1cnnn1CC(=O)OC ; *c1cn(CC(=O)OC)nn1]

or a mixture thereof. In another embodiment, A is

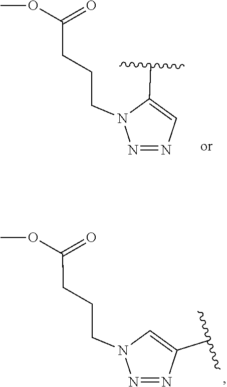 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent.

In any of the above embodiments, the (AA)ₚ group can be modified with one or more enhancement groups. Advantageously, the enhancement group can be linked to the side chain of any amino acid in (AA)ₚ. Useful amino acids for linking enhancement groups include lysine, asparagine, aspartate, glutamine, glutamate, and citrulline. The link to the enhancement group can be a direct bond to the amino acid side chain, or the link can be indirect via a spacer and/or reactive group. Useful spacers and reactive groups include any described above. The enhancement group can be any group deemed useful by those of skill in the art. For example, the enhancement group can be any group that imparts a beneficial effect to the compound, payload, linker payload, or antibody conjugate including, but not limited to, biological, biochemical, synthetic, solubilizing, imaging, detecting, and reactivity effects, and the like. In certain embodiments, the enhancement group is a hydrophilic group. In certain embodiments, the enhancement group is a cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose, and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the enhancement group is capable of improving solublity of the remainder of the conjugate. In certain embodiments, the alkyl, heteroalkyl, alkylenyl, or heteroalkylenyl sulfonic acid is substituted or non-substituted. In certain embodiments, the alkyl, heteroalkyl, alkylenyl, or heteroalkylenyl sulfonic acid is —(CH₂)₁₋₅SO₃H, —(CH₂)—NH—(CH₂)₁₋₅SO₃H, —(CH₂)ₙ—C(O)NH—(CH₂)₁₋₅SO₃H, —(CH₂CH₂O)m-C(O)NH—(CH₂)₁₋₅SO₃H, —(CH₂)ₙ—N((CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5. In some embodiments, the linker is:

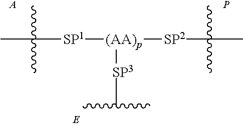

wherein:
SP¹ is a spacer;
SP² is a spacer;
SP³ is a spacer, linked to one AA of (AA)ₚ;
[symbol] is one or more bonds to the binding agent;
[symbol] is one or more bonds to the payload or prodrug payload;
[symbol] is one or more bonds to the enhancement group EG;
each AA is an amino acid; and
p is an integer from zero to ten.
   As discussed above, the bond to the binding agent can be direct, or via a spacer. In certain embodiments, the bond to the binding agent is via a PEG spacer to a glutamine residue of the binding agent.

The SP¹ spacer group is as described above. The SP² spacer group is as described above. Each (AA)ₚ group is as described above.

The SP³ spacer is a moiety that connects the (AA)ₚ moiety to the enhancement group (EG). Suitable SP³ spacers include, but are not limited to, those comprising alkylene or polyether, or both. The ends of the SP³ spacers, i.e., the portion of the SP³ spacer directly bonded to the enhancement group or an AA, can be moieties derived from reactive moieties that are used for purposes of coupling the enhancement group or an AA to the SP³ spacer during the chemical synthesis of the conjugate. In some examples, the ends of the SP³ spacers, i.e., the portion of the spacer directly bonded to the enhancement group or an AA, can be residues of reactive moieties that are used for purposes of coupling the enhancement group or an AA to the spacer during the chemical synthesis of the conjugate. In certain embodiments, SP³ is a spacer, linked to one and only one AA of (AA)ₚ. In certain embodiments, the SP³ spacer is linked to the side chain of a lysine residue of (AA)ₚ.

In some embodiments, the SP³ spacer is:

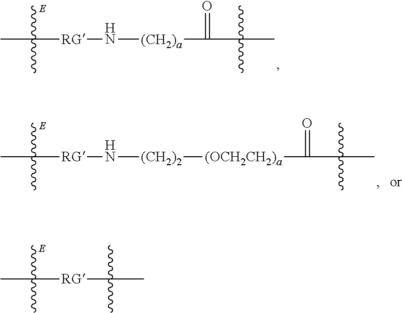

wherein:
RG′ is a reactive group residue following reaction of a reactive group RG with an enhancement agent EG;
[symbol] is a bond to the enhancement agent;
[symbol] is a bond to (AA)ₚ;
a is an integer from 2 to 8; and
p is an integer from zero to four.

The reactive group RG can be any reactive group known to those of skill in the art to be capable of forming one or more bonds to the enhancement agent. The reactive group RG is a moiety comprising a portion in its structure that is capable of reacting with the enhancement group to form a compound of Formula LPa, LPb, LPc, LPd, LPe, LPa′, LPb′, LPc′, LPd′, LPe′, A, B, C, D, E, A′, B′, C′, D′, or E′. Following conjugation to the enhancement group, the reactive group becomes the reactive group residue (RG′). The reactive group RG can be any reactive group described above. Illustrative reactive groups include, but are not limited to, those that comprise haloacetyl, isothiocyanate, succinimide, N-hydroxysuccinimide, or maleimide portions that are capable of reacting with the binding agent.

In certain embodiments, reactive groups include, but are not limited to, alkynes. In certain embodiments, the alkynes are alkynes capable of undergoing 1,3-cycloaddition reactions with azides in the absence of copper catalysts such as strained alkynes. Strained alkynes are suitable for strain-promoted alkyne-azide cycloadditions (SPAAC), cycloalkynes, e.g., cyclooctynes, ane benzannulated alkynes. Suitable alkynes include, but are not limited to, dibenzoazacyclooctyne or

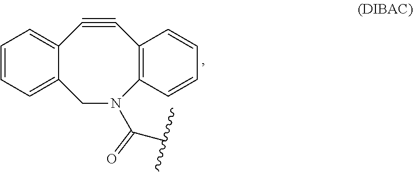 [SMILES: *C(=O)N1Cc2ccccc2C#Cc2ccccc21]

dibenzocyclooctyne or

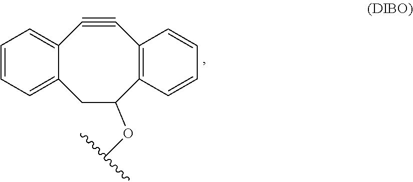 [SMILES: *OC1Cc2ccccc2C#Cc2ccccc21]

biarylazacyclooctynone or

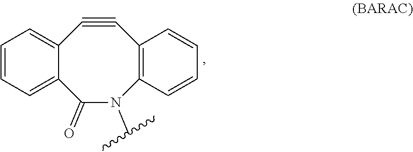 [SMILES: *N1C(=O)c2ccccc2C#Cc2ccccc21]

difluorinated cyclooctyne or

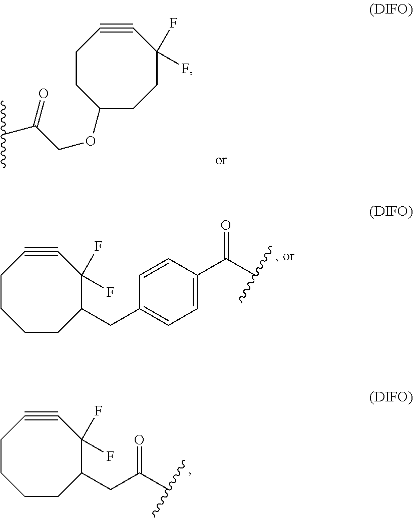 [SMILES: *C(=O)COC1CCC#CC(F)(F)CC1 ; *C(=O)c1ccc(CC2CCCCC#CC2(F)F)cc1 ; *C(=O)CC1CCCCC#CC1(F)F]

substituted, e.g., fluorinated alkynes, aza-cycloalkynes, bicycle[6.1.0]nonyne or

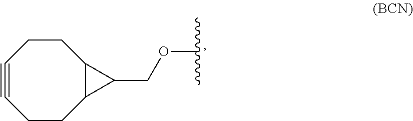 [SMILES: *OCC1C2CCC#CCCC21]

and derivatives thereof. Particularly useful alkynes include

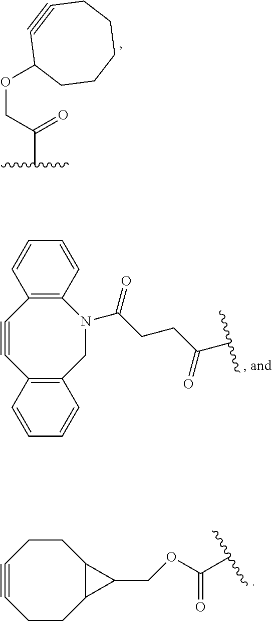 [SMILES: *C(=O)COC1C#CCCCCC1 ; *C(=O)CCC(=O)N1Cc2ccccc2C#Cc2ccccc21 ; *C(=O)OCC1C2CCC#CCCC21]

In some embodiments, the linker is:

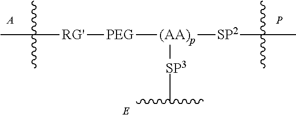

wherein:
RG′ is a reactive group residue following reaction of a reactive group RG with a binding agent;
PEG is —NH-PEG4-C(O)—;
SP² is a spacer;
SP³ is a spacer, linked to one AA residue of (AA)ₚ;
[symbol] is one or more bonds to the binding agent;
[symbol] is one or more bonds to the payload;
[symbol] is one or more bonds to the enhancement group EG;
each AA is an amino acid residue; and
p is an integer from zero to ten.
   As discussed above, the bond to the binding agent can be direct, or via a spacer. In certain embodiments, the bond to the binding agent is via a PEG spacer to a glutamine residue of the binding agent.

In certain embodiments, the linker is:

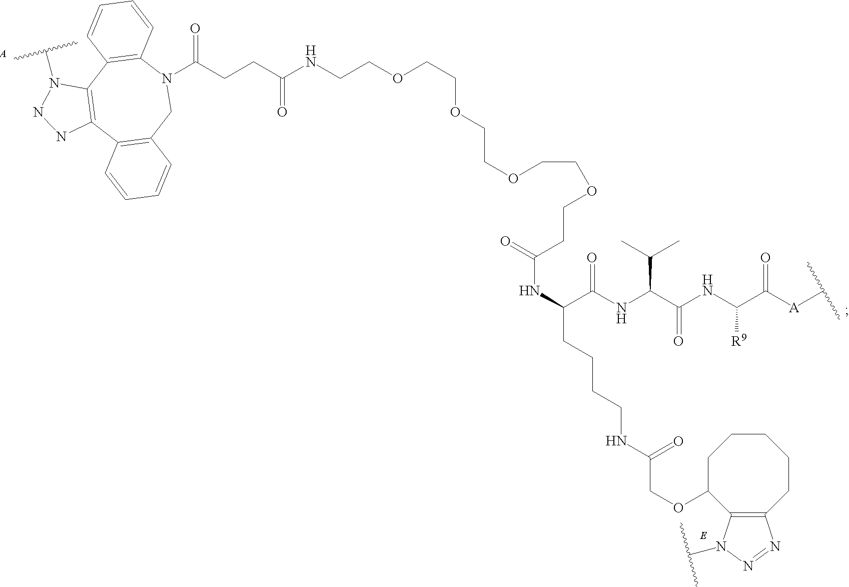

or a pharmaceutically acceptable salt, solvate, or stereoisomeric form thereof, or a regioisomer thereof, or a mixture of regioisomers thereof, wherein:
each [symbol] is a bond to a transglutaminase-modified binding agent;
each [symbol] is a bond to the payload;
each [symbol] is a bond to the enhancement agent;
each R⁹ is —CH₃ or —(CH₂)₃N(H)C(O)NH₂; and
each A is —O—, —N(H)—,

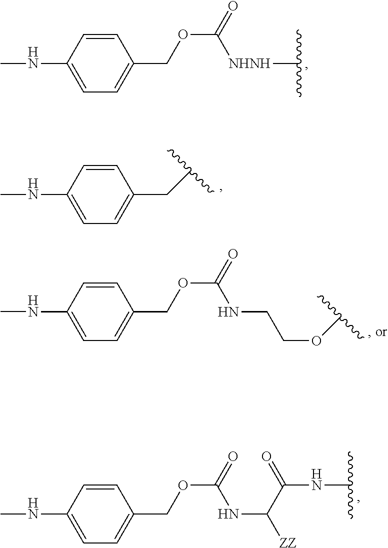

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

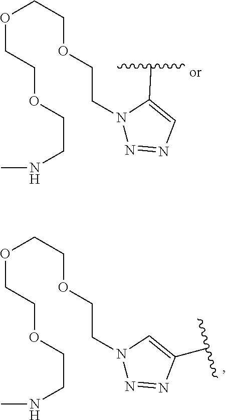 [SMILES: *c1cnnn1CCOCCOCCOCCNC ; *c1cn(CCOCCOCCOCCNC)nn1]

or a mixture thereof. Alternatively, in another embodiment, A is

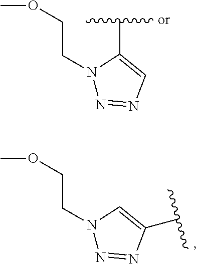

or a mixture thereof. In another embodiment, A is

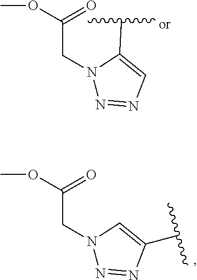 [SMILES: *c1cnnn1CC(=O)OC ; *c1cn(CC(=O)OC)nn1]

or a mixture thereof. In another embodiment, A is

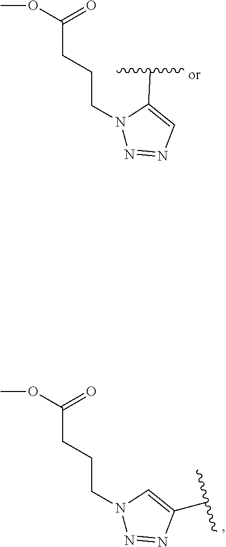 [SMILES: *c1cnnn1CCCC(=O)OC ; *c1cn(CCCC(=O)OC)nn1]

or a mixture thereof. In certain embodiments, 1,3-cycloaddition or SPAAC regioisomers, or mixture of regioisomers, are derived from PEG-N₃ derivitized antibodies treated with suitable alkynes. For example, in one embodiment, the linker is:

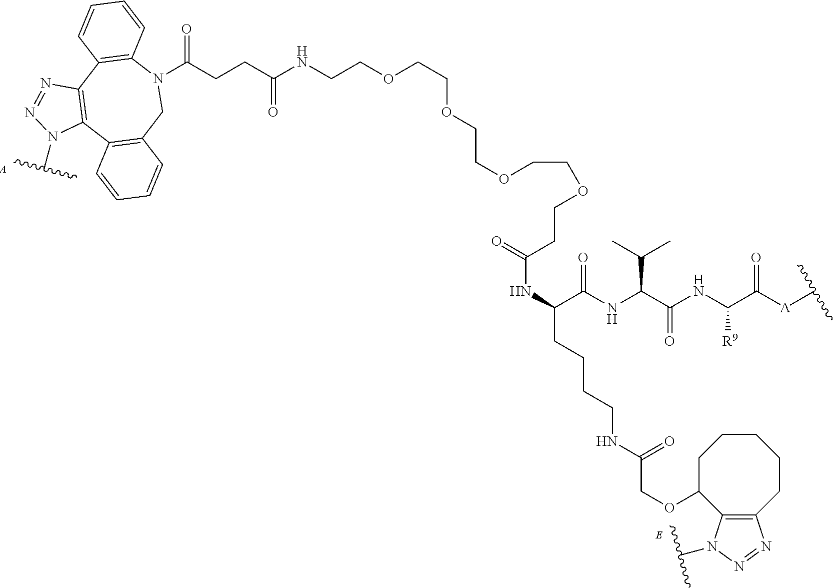

or a pharmaceutically acceptable salt, solvate, or stereoisomeric form thereof, or a regioisomer thereof, or a mixture of regioisomers thereof. By way of further example, in one embodiment, the linker is:

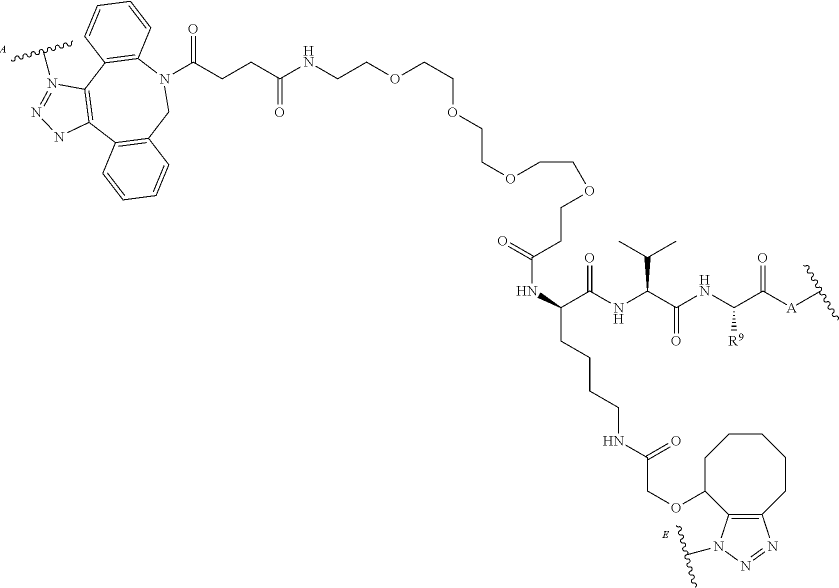

or a pharmaceutically acceptable salt, solvate, or stereoisomeric form thereof, or a regioisomer thereof, or a mixture of regioisomers thereof. By way of further example, the linker is:

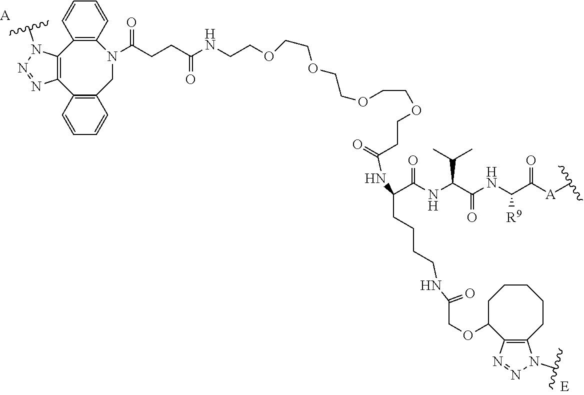

or a pharmaceutically acceptable salt, solvate, or stereoisomeric form thereof, or a regioisomer thereof, or a mixture of regioisomers thereof. By way of further example, in one embodiment, the linker is:

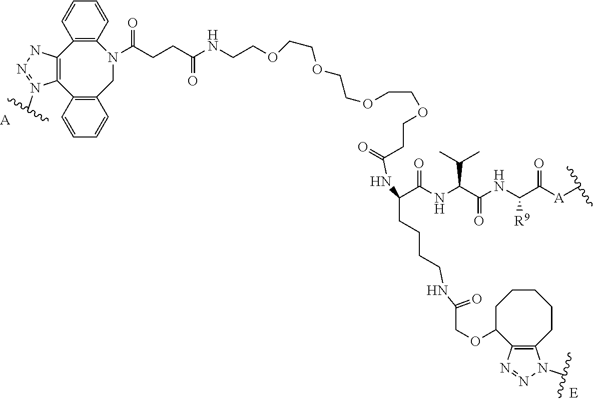

or a pharmaceutically acceptable salt, solvate, or stereoisomeric form thereof, or a regioisomer thereof, or a mixture of regioisomers thereof. As discussed above, the bond to the binding agent can be direct, or via a spacer. In certain embodiments, the bond to the binding agent is via a PEG spacer to a glutamine residue of the binding agent. In certain embodiments, the enhancement agent is a hydrophilic group. In certain embodiments, the enhancement agent is cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose, and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the alkyl, heteroalkyl, alkylenyl, or heteroalkylenyl sulfonic acid is —(CH₂)₁₋₅SO₃H, —(CH₂)—NH—(CH₂)₁—SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂)₁₋₅C(O)NH(CH₂)₁₋₅SO₃H)₂, —(CH₂)ₙ—C(O)N((CH₂)₁₋₅C(O)NH(CH₂)i-5SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5.

In some embodiments, the linker is:

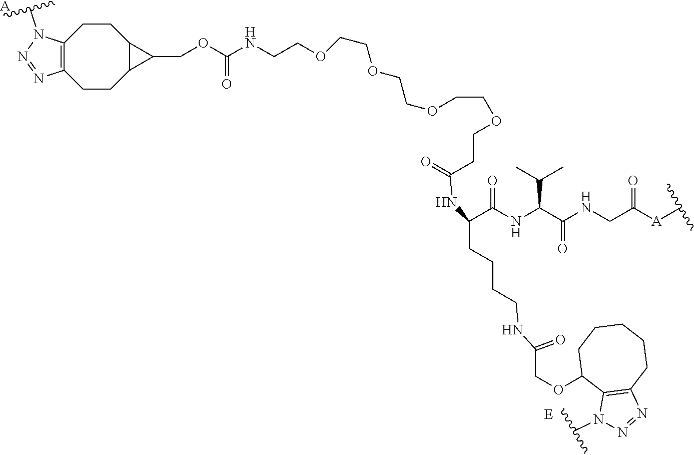

or a pharmaceutically acceptable salt, solvate, or stereoisomeric form thereof, or a regioisomer thereof, or mixture of regioisomers thereof, wherein:
each [symbol] is a bond to a transglutaminase-modified binding agent;
each [symbol] is a bond to the enhancement agent;
each [symbol] is a bond to the payload;
each R⁹ is —CH₃ or —(CH₂)₃N(H)C(O)NH₂; and
each A is —O—, —N(H)—,

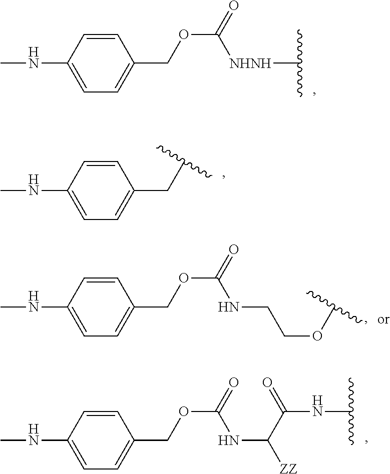

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

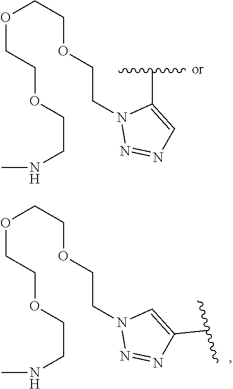

or a mixture thereof. Alternatively, in another embodiment, A is

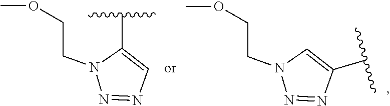

or a mixture thereof. In another embodiment, A is

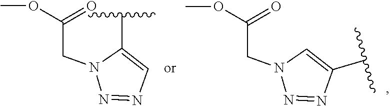 [SMILES: *c1cnnn1CC(=O)OC ; *c1cn(CC(=O)OC)nn1]

or a mixture thereof. In another embodiment, A is

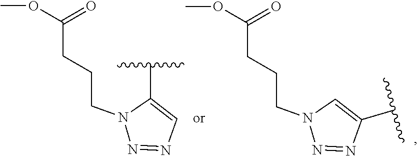 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent. In certain embodiments, the enhancement agent is a hydrophilic group. In certain embodiments, the enhancement agent is cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose, and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the alkyl, heteroalkyl, alkylenyl, or heteroalkylenyl sulfonic acid is —(CH₂)₁₋₅SO₃H, —(CH₂)—NH—(CH₂)₁—SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂)₁₋₅C(O)NH(CH₂)₁₋₅SO₃H)₂, —(CH₂)ₙ—C(O)N((CH₂)₁₋₅C(O)NH(CH₂)i-5SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5.

In some embodiments, the linker is:

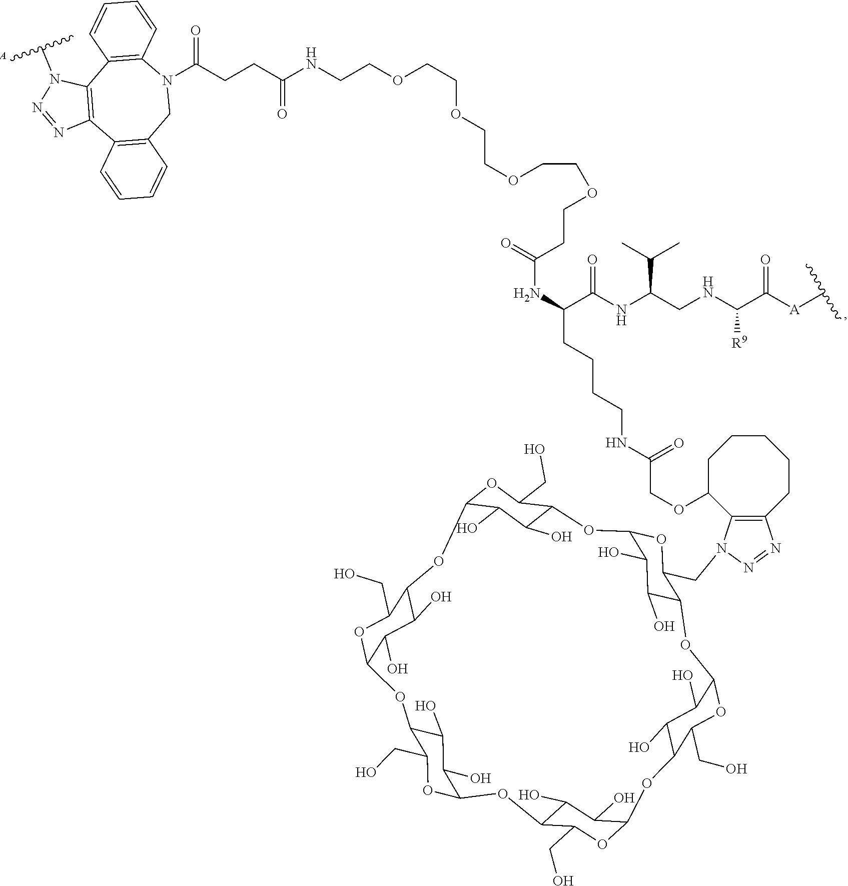

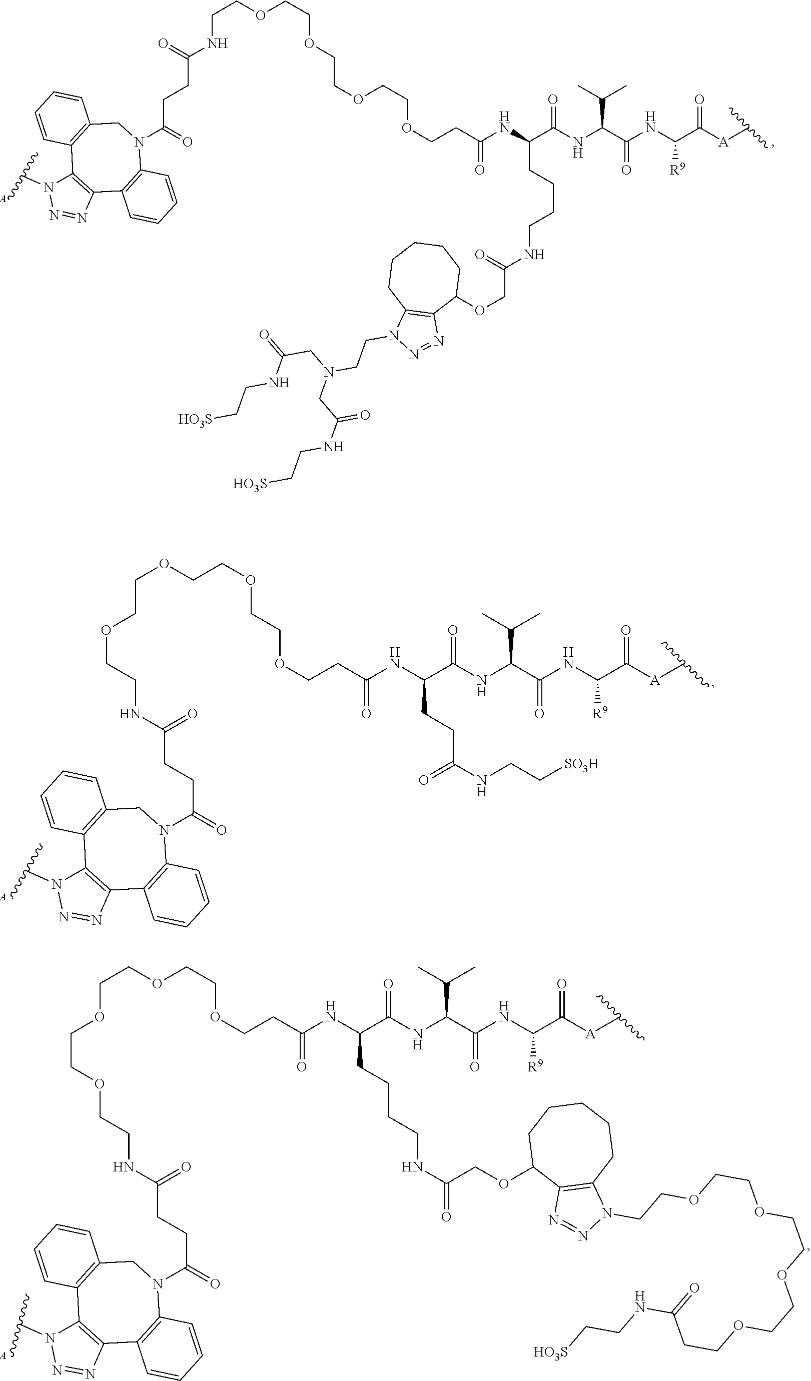

or a pharmaceutically acceptable salt, solvate, or stereoisomeric form thereof, or a regioisomer thereof, or mixture of regioisomers thereof, wherein:
each [symbol] is a bond to a transglutaminse-modified binding agent;
each [symbol] is a bond to the payload;
R⁹ is —CH₃ or —(CH₂)₃N(H)C(O)NH₂; and
A is —O—, —N(H)—,

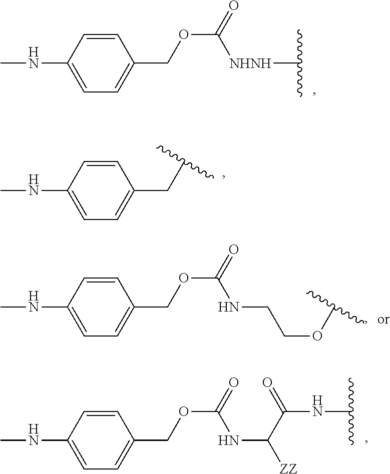

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

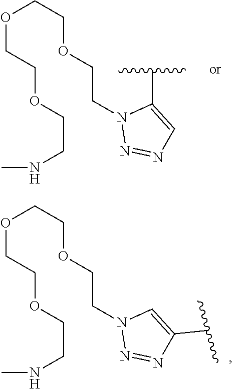

or a mixture thereof. Alternatively, in another embodiment, A is

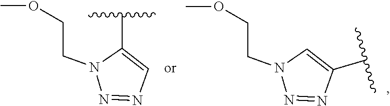

or a mixture thereof. In another embodiment, A is

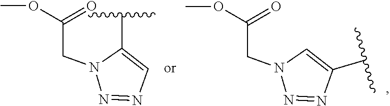 [SMILES: *c1cnnn1CC(=O)OC ; *c1cn(CC(=O)OC)nn1]

or a mixture thereof. In another embodiment, A is

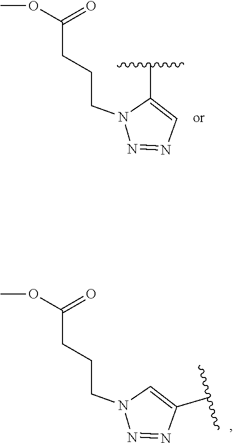 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent.

In some embodiments, the linker is:

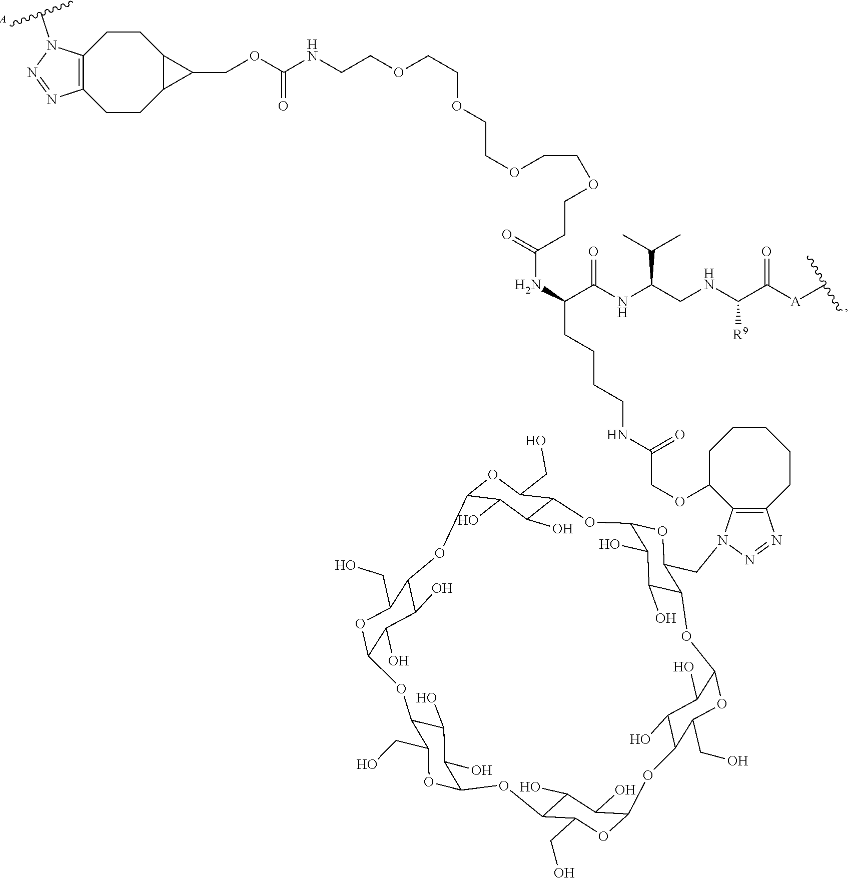

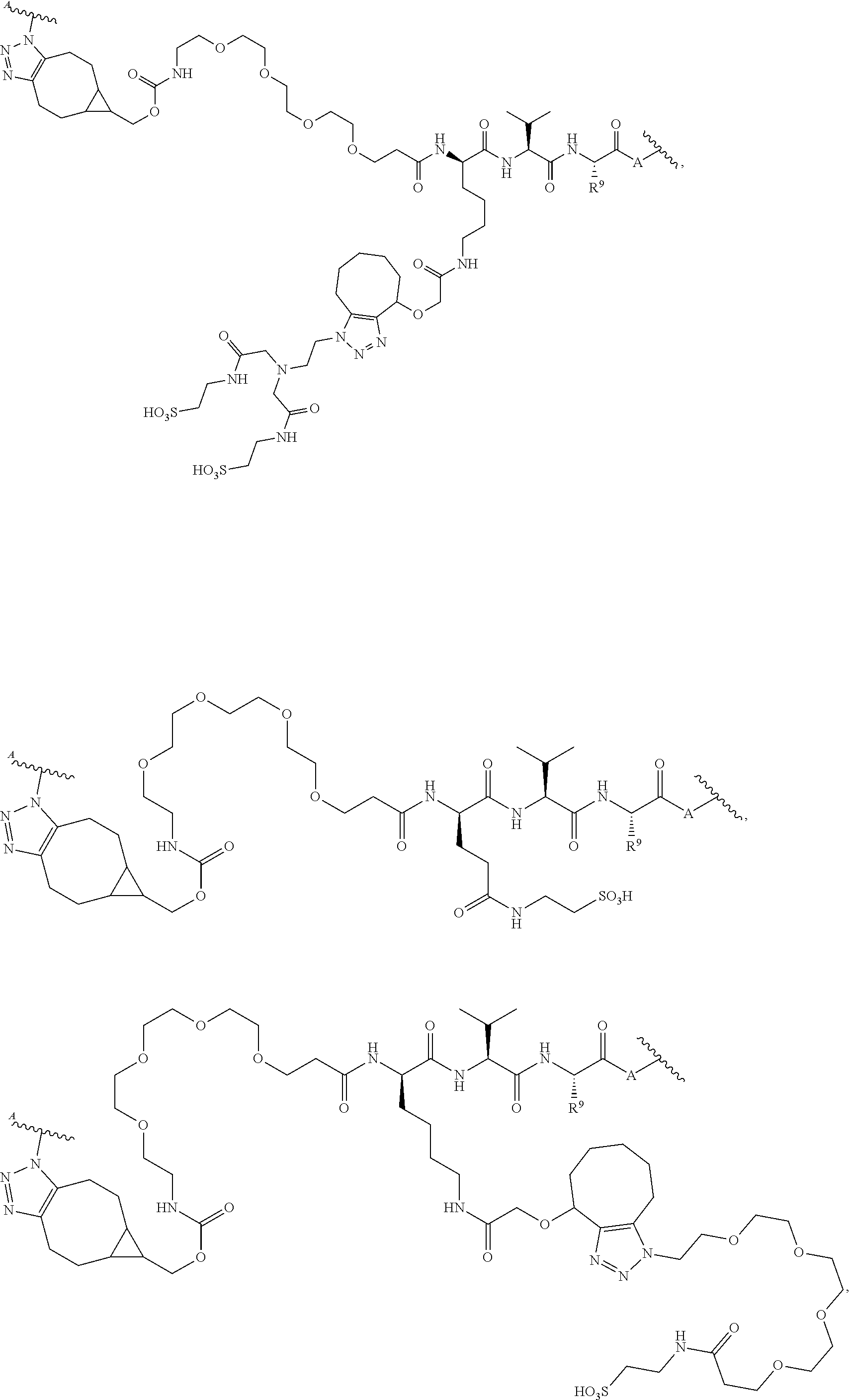

or a pharmaceutically acceptable salt, solvate, or stereoisomeric form thereof, or a regioisomer thereof, or mixture of regioisomers thereof, wherein:
each [symbol] is a bond to a transglutaminse-modified binding agent;
each [symbol] is a bond to the payload;
R⁹ is —CH₃ or —(CH₂)₃N(H)C(O)NH₂; and
A is —O—, —N(H)—,

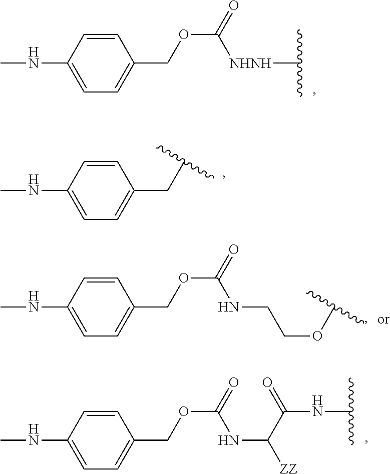

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

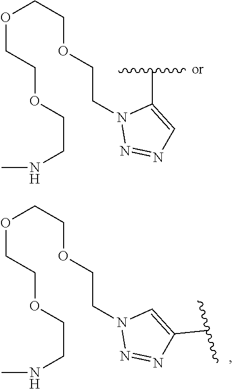

or a mixture thereof. Alternatively, in another embodiment, A is

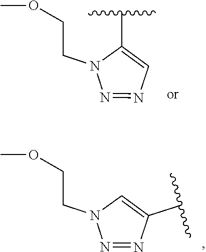

or a mixture thereof. In another embodiment, A is

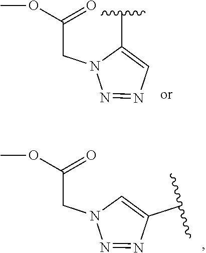 [SMILES: *c1cnnn1CC(=O)OC ; *c1cn(CC(=O)OC)nn1]

or a mixture thereof. In another embodiment, A is

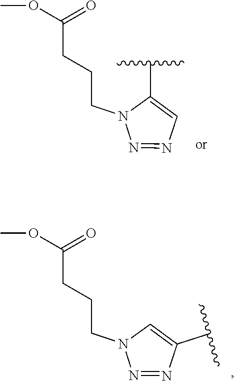 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent.

In some embodiments, the linker is:

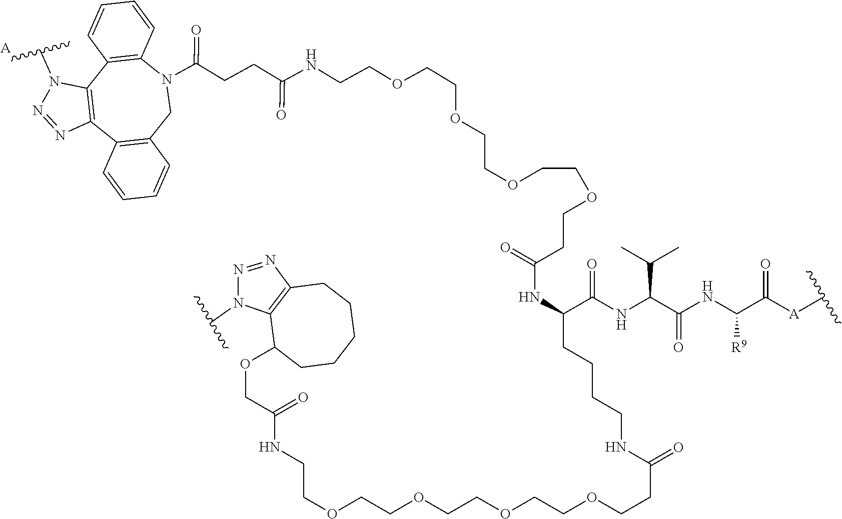

or a pharmaceutically acceptable salt, solvate, or stereoisomeric form thereof, or a regioisomer thereof, or mixture of regioisomers thereof, wherein:
each [symbol] is a bond to a transglutaminse-modified binding agent;
each [symbol] is a bond to the payload;
each [symbol] is a bond to the enhancement group;
each R⁹ is —CH₃ or —(CH₂)₃N(H)C(O)NH₂; and
each A is —O—, —N(H)—,

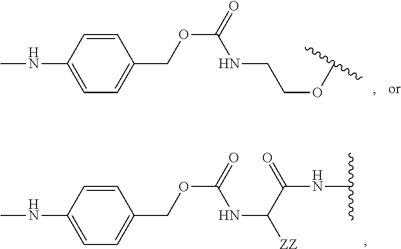

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

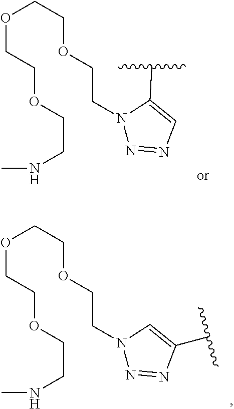

or a mixture thereof. Alternatively, in another embodiment, A is

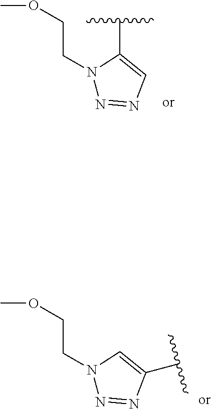

or a mixture thereof. In another embodiment, A is

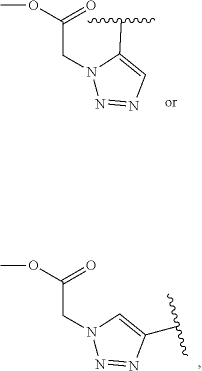 [SMILES: *c1cnnn1CC(=O)OC ; *c1cn(CC(=O)OC)nn1]

or a mixture thereof. In another embodiment, A is

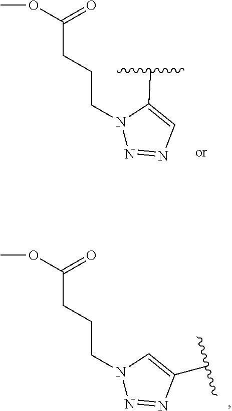 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent. In certain embodiments, the enhancement agent is a hydrophilic group. In certain embodiments, the enhancement agent is cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose, and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the alkyl, heteroalkyl, alkylenyl, or heteroalkylenyl sulfonic acid is —(CH₂)₁₋₅SO₃H, —(CH₂)—NH—(CH₂)₁—SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂)₁₋₅C(O)NH(CH₂)₁₋₅SO₃H)₂, —(CH₂)ₙ—C(O)N((CH₂)₁₋₅C(O)NH(CH₂)i-5SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5.

In some embodiments, the linker is:

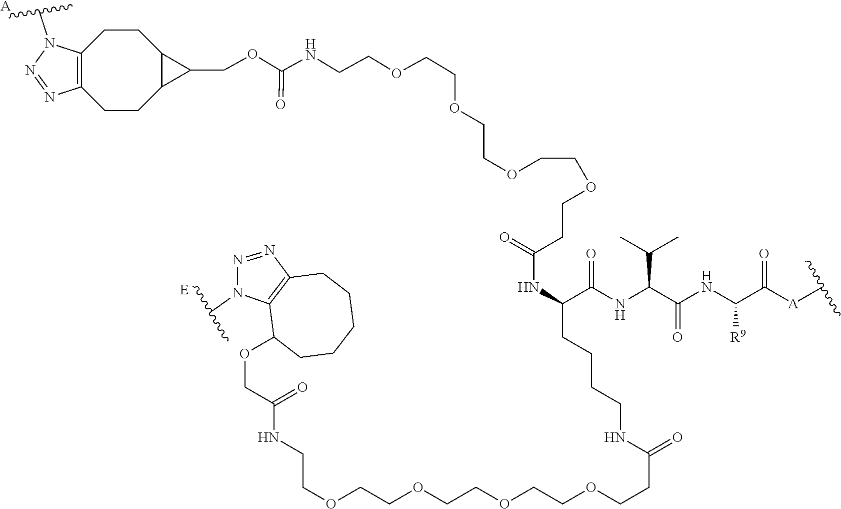

or a pharmaceutically acceptable salt, solvate, or stereoisomeric form thereof, or a regioisomer thereof, or mixture of regioisomers thereof, wherein:
each [symbol] is a bond to a transglutaminase-modified binding agent;
each [symbol] is a bond to the payload;
each R⁹ is —CH₃ or —(CH₂)₃N(H)C(O)NH₂; and
each A is —O—, —N(H)—,

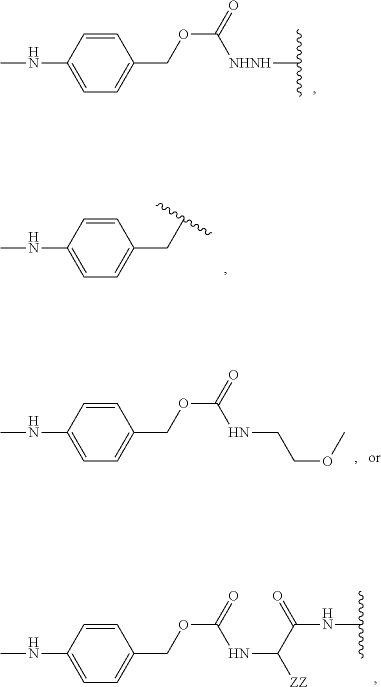

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

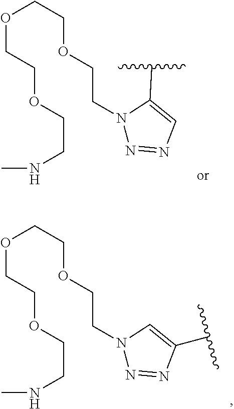 [SMILES: *c1cnnn1CCOCCOCCOCCNC ; *c1cn(CCOCCOCCOCCNC)nn1]

or a mixture thereof. Alternatively, in another embodiment, A is

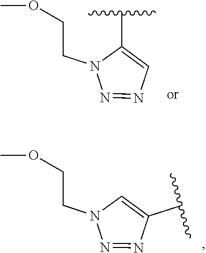

or a mixture thereof. In another embodiment, A is

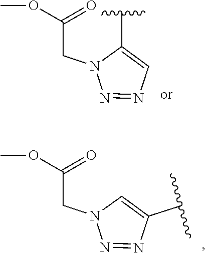 [SMILES: *c1cnnn1CC(=O)OC ; *c1cn(CC(=O)OC)nn1]

or a mixture thereof. In another embodiment, A is

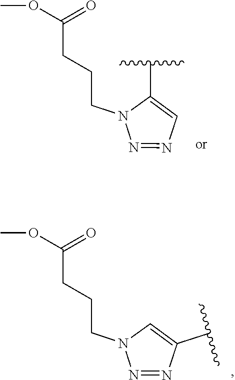 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent. In certain embodiments, the enhancement agent is a hydrophilic group. In certain embodiments, the enhancement agent is cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose, and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the alkyl, heteroalkyl, alkylenyl, or heteroalkylenyl sulfonic acid is —(CH₂)₁₋₅SO₃H, —(CH₂)—NH—(CH₂)₁—SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂)₁₋₅C(O)NH(CH₂)i-5SO₃H)₂, —(CH₂)ₙ—C(O)N((CH₂)₁₋₅C(O)NH(CH₂)i-5SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5.

In some embodiments, the linker is:

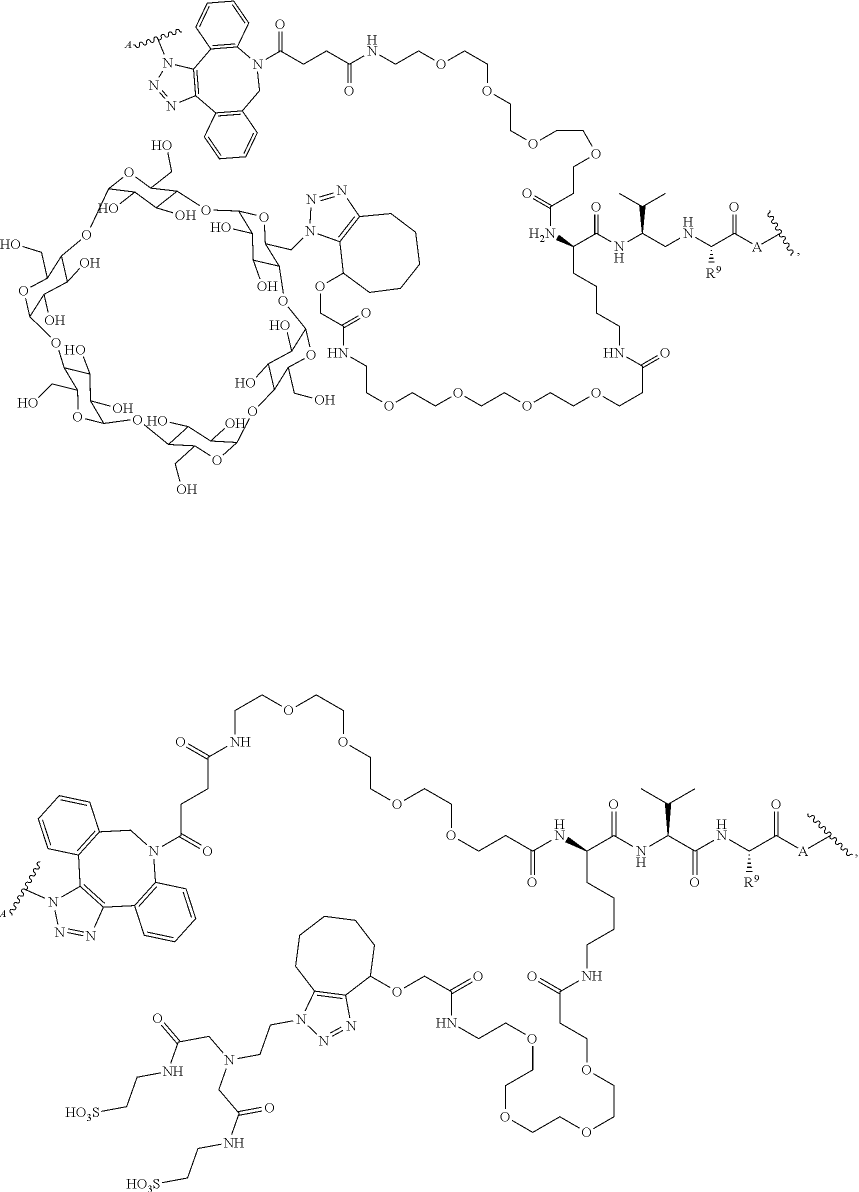

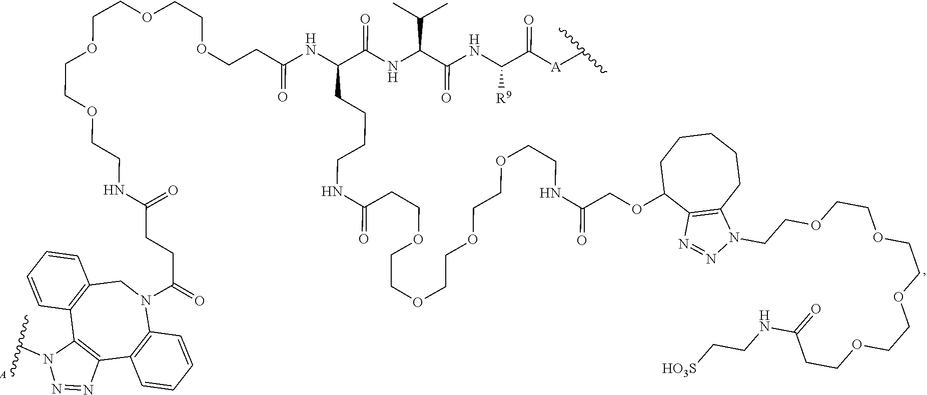

or a pharmaceutically acceptable salt, solvate, or stereoisomeric form thereof, or a regioisomer thereof, or mixture of regioisomers thereof, wherein:
each [symbol] is a bond to a transglutaminanse-modified binding agent;
each [symbol] is a bond to the payload;

R⁹ is —CH₃ or —(CH₂)₃N(H)C(O)NH₂; and
A is —O—, —N(H)—,

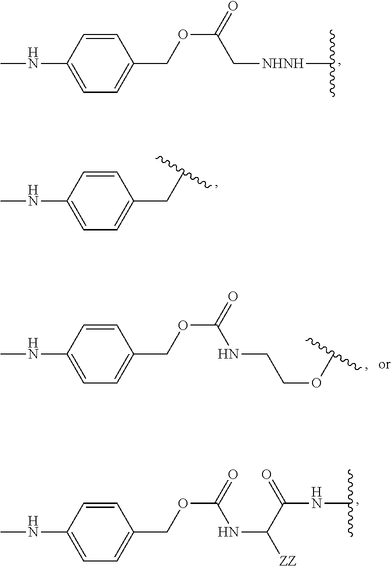

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

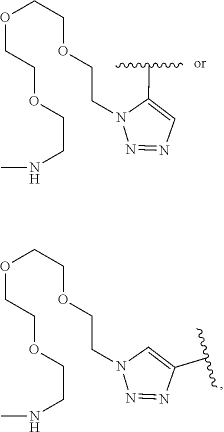 [SMILES: *c1cnnn1CCOCCOCCOCCNC ; *c1cn(CCOCCOCCOCCNC)nn1]

or a mixture thereof. Alternatively, in another embodiment, A is

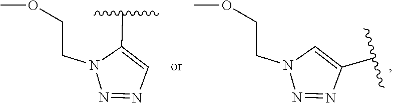

or a mixture thereof. In another embodiment, A is

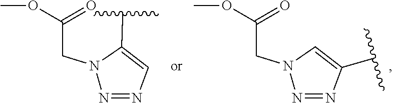 [SMILES: *c1cnnn1CC(=O)OC ; *c1cn(CC(=O)OC)nn1]

or a mixture thereof. In another embodiment, A is

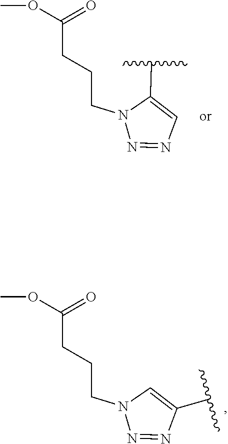 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent.

In some embodiments, the linker is:

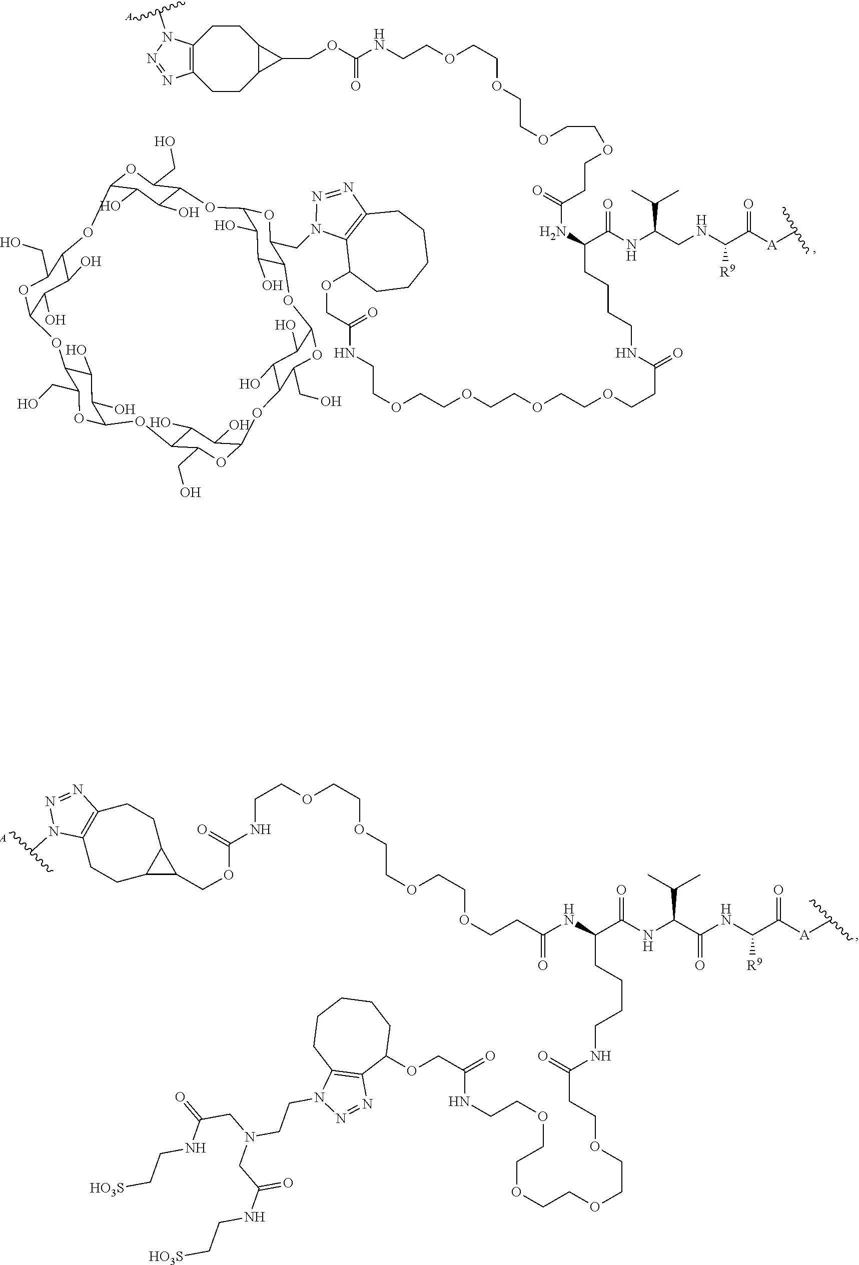

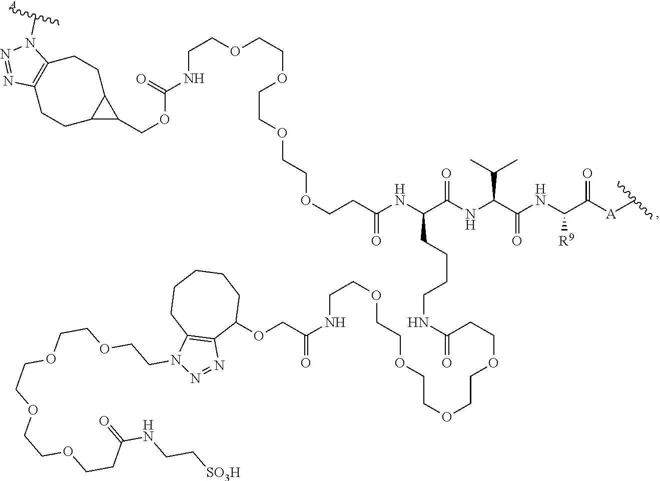

or a pharmaceutically acceptable salt, solvate, or stereoisomeric form thereof, or a regioisomer thereof, or mixture of regioisomers thereof, wherein:
each [symbol] is a bond to a transglutaminase-modified binding agent;
each [symbol] is a bond to the payload;
R⁹ is —CH₃ or —(CH₂)₃N(H)C(O)NH₂; and
A is —O—, —N(H)—,

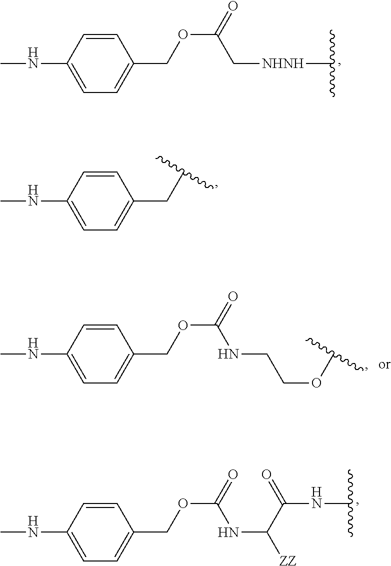

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

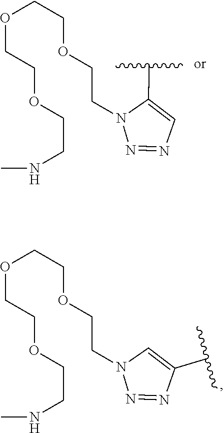 [SMILES: *c1cnnn1CCOCCOCCOCCNC ; *c1cn(CCOCCOCCOCCNC)nn1]

or a mixture thereof. Alternatively, in another embodiment, A is

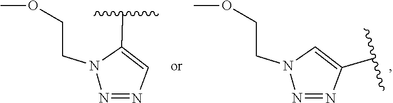

or a mixture thereof. In another embodiment, A is

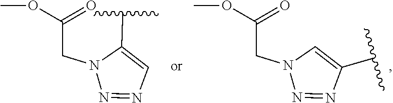 [SMILES: *c1cnnn1CC(=O)OC ; *c1cn(CC(=O)OC)nn1]

or a mixture thereof. In another embodiment, A is

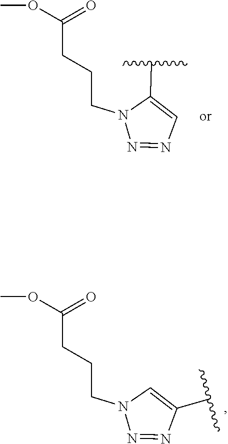 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent.

In particular embodiments, disclosed compounds, payloads, or prodrug payloads with an alkyne or terminal acetylene may be linked to a binding agent derivatized with -PEG-N₃ linked to a glutamine residue (viz. a transglutaminase-modified binding agent). Exemplary —N₃ derivatized binding agents (viz., transglutaminase-modified binding agents), methods for their preparation, and methods for their use are provided herein. In certain embodiments, a compound or payload with an alkyne described herein suitable for participation in 1,3-cycloadditions with binding agents derivatized with -PEG-N₃ provide regioisomeric 1,2,3-triazolyl linked moieties. For example, in certain embodiments, compounds or payloads linked to the binding agent may be

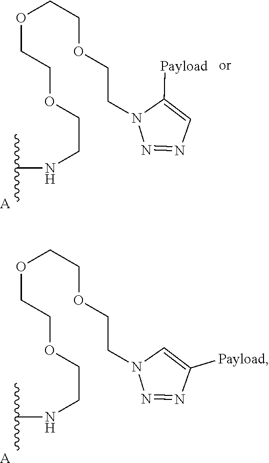

or a mixture thereof, where each [symbol] is a bond to the binding agent.

### Linker-Payloads

In certain embodiments, linker-payloads or linker-prodrug payloads (i.e., these descriptors are interchangeably used throughout) include any specific compound embraced by any one or more of Formulae I, Ia, II, III, IV, V, or VI above, bonded to a linker, wherein the linker(s) described herein include a moiety that is reactive with an antibody or antigen binding fragment thereof described herein. In particular embodiments, the linker is bonded to a heterocycle comprising nitrogen, R¹, R², R³, R⁶, or R⁷ in any one or more of Formulae I, Ia, II, III, IV, V, or VI above.

In one embodiment, the linker-payload has a Formula LPa, LPb, LPc, LPd, or LPe

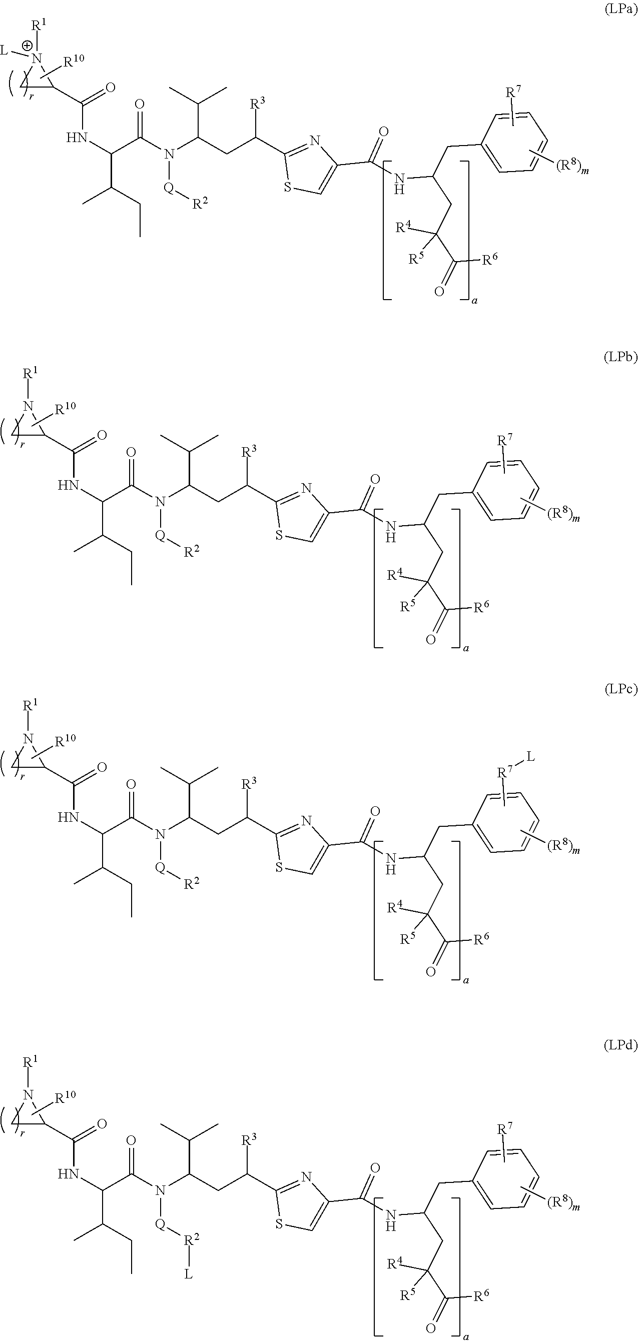

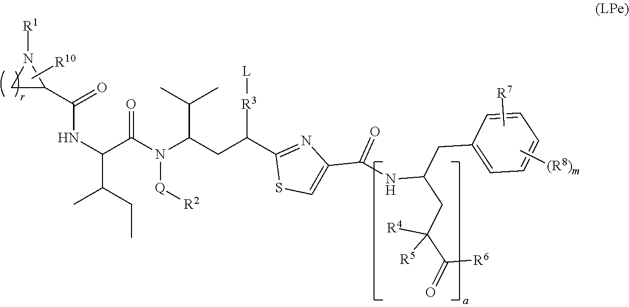

wherein L is a linker.

In one embodiment, the linker-payload has a Formula LPa, LPb, LPc, LPd, or LPe, wherein

L is a linker; and R⁷ is, independently in each instance, hydrogen, —OH, —O—, halogen, or —NR^{7a}R^{7b}, wherein R^{7a} and R^{7b} are, independently in each instance, a bond, hydrogen, alkyl, alkenyl, alkynyl, cycloalkyl, aryl, heteroaryl, acyl, —C(O)CH₂OH, —C(O)CH₂O—, a first N-terminal amino acid residue, a first N-terminal peptide residue, —CH₂CH₂NH₂, and —CH₂CH₂NH—, wherein alkyl, alkenyl, alkynyl, cycloalkyl, aryl, heteroaryl, and acyl are optionally substituted.

In one embodiment, the linker-payload has a structure of Formula LPa′

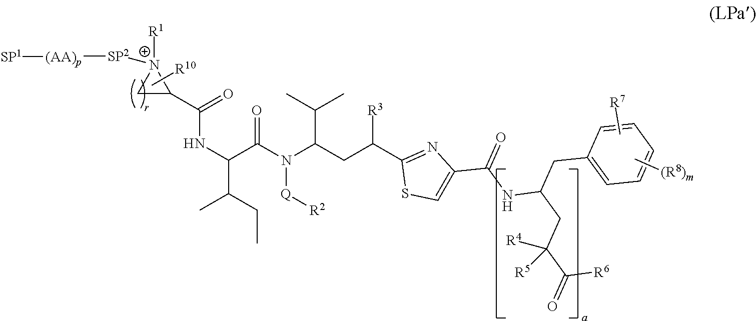

wherein SP′, (AA)ₚ, SP², R¹, Q, R², R³, R⁴, R⁵, R⁶, R⁷, R⁸, R¹⁰, r, and a are as described in any of the embodiments disclosed herein. In one embodiment, the linker-payload has a structure of Formula LPb′

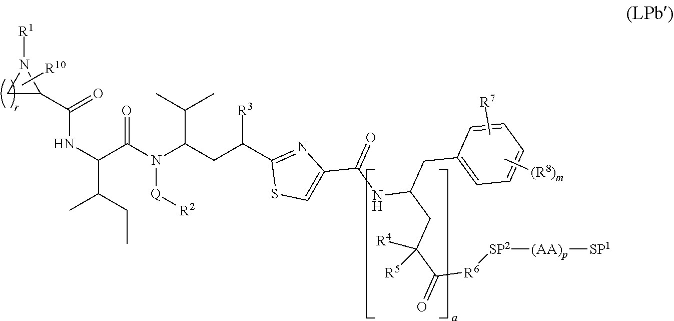

wherein SP¹, (AA)ₚ, SP², R¹, Q, R², R³, R⁴, R⁵, R⁶, R⁷, R⁸, R¹⁰, r, and a are as described in any of the embodiments disclosed herein. In one embodiment, the linker-payload has a structure of Formula LPc′

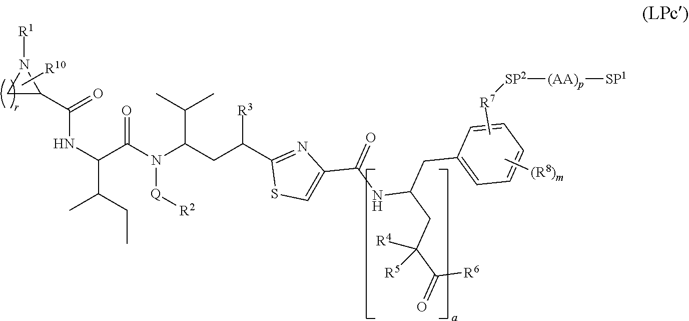

wherein SP¹, (AA)ₚ, SP², R¹, Q, R², R³, R⁴, R, R⁶, R⁷, R⁸, R¹⁰, r, and a are as described in any of the embodiments disclosed herein. In one embodiment, the linker-payload has a structure of Formula LPd′

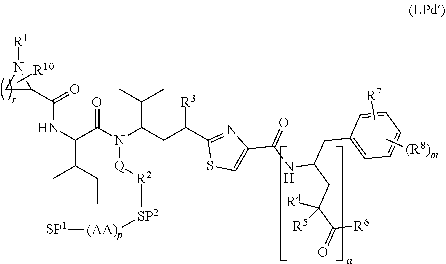

wherein SP¹, (AA)ₚ, SP², R¹, Q, R², R³, R⁴, R⁵, R⁶, R⁷, R⁸, R¹⁰, r, and a are as described in any of the embodiments disclosed herein. In one embodiment, the linker-payload has a structure of Formula LPe′

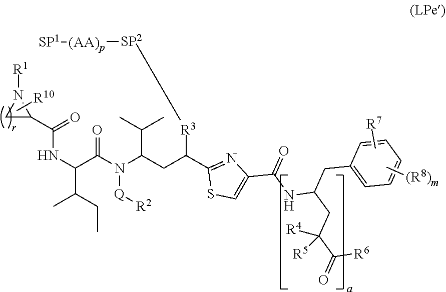

wherein SP′, (AA)ₚ, SP², R¹, Q, R², R³, R⁴, R⁵, R⁶, R⁷, R⁸, R¹⁰, r, and a are as described in any of the embodiments disclosed herein. In any of the embodiments in this paragraph, Formulae LPa′, LPb′, LPc′, LPd′, or LPe′ may be a pharmaceutically acceptable salt or prodrug thereof. In any of the embodiments in this paragraph, p is zero, one, two, three, four, five, six, seven, eight, nine, or ten. In one embodiment, the linker-payload has a structure of LPa′, LPb′, LPc′, LPd′, or LPe′, wherein the —SP²— spacer, when present, is

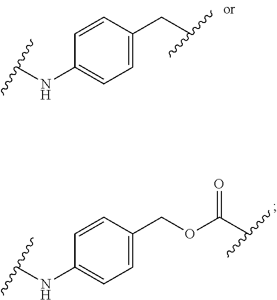 [SMILES: *Cc1ccc(N*)cc1 ; *Nc1ccc(COC(*)=O)cc1]

the second -(AA)ₚ- is

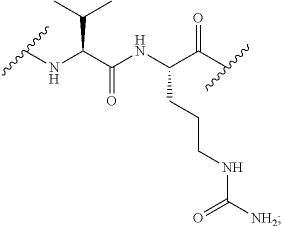 [SMILES: *N[C@H](C(=O)N[C@@H](CCCNC(N)=O)C(*)=O)C(C)C]

the —SP¹— spacer is

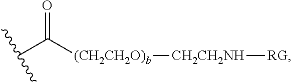

wherein RG is a reactive group; and b is an integer from one to four. In one embodiment, the linker-payload has a structure of LPa′, LPb′, LPc′, LPd′, or LPe′, wherein Q is —O—. In one embodiment, the linker-payload has a structure of LPa′, LPb′, LPc′, LPd′, or LPe′, wherein Q is —CH₂—; R¹ is C₁-C₁₀ alkyl; R² is alkyl; R⁴ and R⁵ are C₁-C₅ alkyl; R⁶ is —OH; R¹⁰ is absent; wherein r is four; and wherein a is one. In one embodiment, the linker-payload has a structure of LPc′, or a pharmaceutically acceptable salt thereof. In one embodiment, the linker-payload has a structure of LPc′, or a pharmaceutically acceptable salt thereof, wherein R⁷ is —NH—; and R⁸ is hydrogen or fluoro. In one embodiment, the linker-payload has a structure of LPc′, or a pharmaceutically acceptable salt thereof, wherein R⁷ is —NH—; and R⁸ is hydrogen. In one embodiment, the linker-payload has a structure of LPc′, or a pharmaceutically acceptable salt thereof, wherein R⁷ is —NH—; and R⁸ is fluoro. In one embodiment, the linker-payload has a structure of LPe′, or a pharmaceutically acceptable salt thereof. In one embodiment, the linker-payload has a structure of LPe′, or a pharmaceutically acceptable salt thereof, wherein R³ is —OC(O)N(H)CH₂CH₂NH—or —OC(O)N(H)CH₂CH₂OCH₂CH₂OCH₂CH₂OCH₂CH₂NH—. In one embodiment, the linker-payload has a structure of LPe′, or a pharmaceutically acceptable salt thereof, wherein R³ is —OC(O)N(H)CH₂CH₂NH—. In one embodiment, the linker-payload has a structure of LPe′, or a pharmaceutically acceptable salt thereof, wherein R³ is —OC(O)N(H)CH₂CH₂OCH₂CH₂OCH₂CH₂OCH₂CH₂NH—. In one embodiment, the linker-payload has a structure of LPa′, LPb′, LPc′, LPd′, or LPe′, wherein Q is —CH₂—; R¹ is hydrogen or C₁-C₁₀ alkyl; R² is alkyl; R⁴ and R⁵ are C₁-C₅ alkyl; R⁶ is —OH; wherein r is three or four; and wherein a is one. In one embodiment, the linker-payload has a structure of LPc′, or a pharmaceutically acceptable salt thereof. In one embodiment, the linker-payload has a structure of LPc′, or a pharmaceutically acceptable salt thereof, wherein R⁷ is —NH—; and R⁸ is hydrogen. In one embodiment, the linker-payload has a structure of LPa′, LPb′, LPc′, LPd′, or LPe′, wherein Q is —CH₂—; R¹ is hydrogen or C₁-C₁₀ alkyl; R² is alkyl; R⁴ and R⁵ are C₁-C₅ alkyl; R⁶ is —OH; R⁰ is absent; wherein r is four; and wherein a is one. In one embodiment, the linker-payload has a structure of LPc′, or a pharmaceutically acceptable salt thereof. In one embodiment, the linker-payload has a structure of LPc′, or a pharmaceutically acceptable salt thereof, wherein R⁷ is —NH—; and R⁸ is hydrogen. In one embodiment, the linker-payload has a structure of LPa′, LPb′, LPc′, LPd′, or LPe′, wherein Q is —O—; R¹ is hydrogen or C₁-C₁₀ alkyl; R² is alkyl or alkynyl; R³ is hydroxyl or —OC(O)C₁-C₅ alkyl; R⁴ and R⁵ are C₁-C₅ alkyl; R⁶ is —OH; R⁰, when present, is —C₁-C₅ alkyl; wherein r is three or four; and wherein a is one. In one embodiment, the linker-payload has a structure of LPc′, or a pharmaceutically acceptable salt thereof. In one embodiment, the linker-payload has a structure of LPc′, or a pharmaceutically acceptable salt thereof, wherein R⁷ is —NH—; and R⁸ is hydrogen. In one embodiment, the linker-payload has a structure of LPa′, LPb′, LPc′, LPd′, or LPe′, wherein Q is —CH₂— or —O—; R¹ is C₁-C₁₀ alkyl; R² is alkyl or alkynyl; R⁴ and R⁵ are C₁-C₅ alkyl; R⁶ is —NHSO₂(CH₂)ₐ₁-aryl-(CH₂)ₐ₂NR^{6a}R^{6b};
R¹⁰ is absent; wherein r is four; and wherein a, a1, and, a2 are, independently, zero or one. In one embodiment, the linker-payload has a structure of LPb′, or a pharmaceutically acceptable salt thereof. In one embodiment, the linker-payload has a structure of LPb′, or a pharmaceutically acceptable salt thereof, wherein R⁶ is

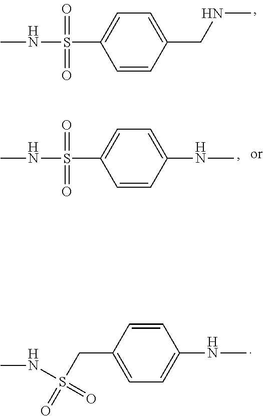

In one embodiment, the linker-payload has a structure of LPb′, or a pharmaceutically acceptable salt thereof, wherein R⁶ is

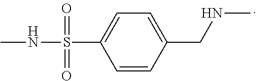

In one embodiment, the linker-payload has a structure of LPb′, or a pharmaceutically acceptable salt thereof, wherein R⁶ is

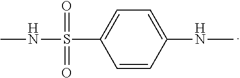

In one embodiment, the linker-payload has a structure of LPb′, or a pharmaceutically acceptable salt thereof, wherein R⁶ is

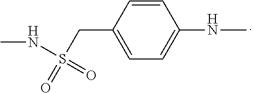

In one embodiment, the linker-payload has a structure of LPb′, or a pharmaceutically acceptable salt thereof, wherein a is zero; and R⁶ is

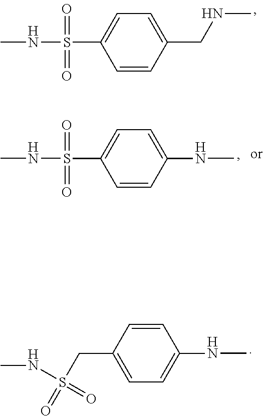

In one embodiment, the linker-payload has a structure of LPb′, or a pharmaceutically acceptable salt thereof, wherein a is zero; and R⁶ is

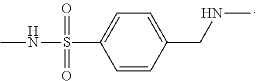

In one embodiment, the linker-payload has a structure of LPb′, or a pharmaceutically acceptable salt thereof, wherein a is zero; and R⁶ is

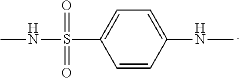

In one embodiment, the linker-payload has a structure of LPb′, or a pharmaceutically acceptable salt thereof, wherein a is zero; and R⁶ is

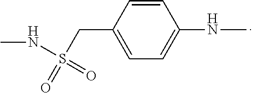

In one embodiment, the linker-payload has a structure of LPb′, or a pharmaceutically acceptable salt thereof, wherein a is one; and R⁶ is

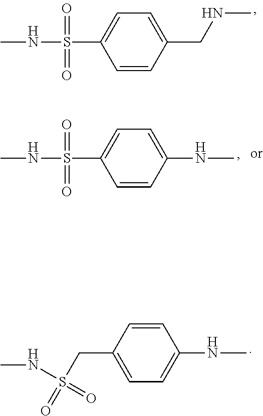

In one embodiment, the linker-payload has a structure of LPb′, or a pharmaceutically acceptable salt thereof, wherein a is one; and R⁶ is

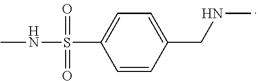

In one embodiment, the linker-payload has a structure of LPb′, or a pharmaceutically acceptable salt thereof, wherein a is one; and R⁶ is

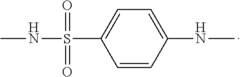

In one embodiment, the linker-payload has a structure of LPb′, or a pharmaceutically acceptable salt thereof, wherein a is one; and R⁶ is

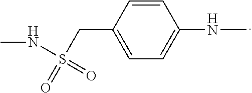

In one embodiment, the linker-payload has a structure of LPc′, or a pharmaceutically acceptable salt thereof, wherein R⁷ is —O—; and R⁸ is hydrogen.

In any of the foregoing embodiments, aryl includes phenyl, naphthyl, fluorenyl, azulenyl, anthryl, phenanthryl, and pyrenyl; heteroaryl includes furanyl, thiophenyl, pyrrolyl, oxazolyl, thiazolyl, imidazolyl, pyrazolyl, isoxazolyl, isothiazolyl, pyridyl, pyrazinyl, pyrimidinyl, pyridazinyl, quinolinyl, isoquinolinyl, cinnolinyl, quinazolinyl, quinoxalinyl, phthalazinyl, pteridinyl, benzofuranyl, dibenzofuranyl, benzothiophenyl, benzoxazolyl, benzthiazoyl, dibenzothiophenyl, indolyl, indolinyl, benzimidazolyl, indazolyl, and benztriazolyl; a heterocycle comprising nitrogen includes aziridinyl, azetidinyl, pyrrolidinyl, piperidinyl, azepanyl, and azocanyl; and acyl includes —C(O)R^{3c}, wherein R^{3c} comprises alkyl, alkenyl, alkynyl, cycloalkyl, aryl, and heteroaryl. In one embodiment, aryl is phenyl. In one embodiment, aryl is naphthyl. In one embodiment, aryl is fluorenyl. In one embodiment, aryl is azulenyl. In one embodiment, aryl is anthryl. In one embodiment, aryl is phenanthryl. In one embodiment, aryl is pyrenyl. In one embodiment, heteroaryl is furanyl. In one embodiment, heteroaryl is thiophenyl. In one embodiment, heteroaryl is pyrrolyl. In one embodiment, heteroaryl is oxazolyl. In one embodiment, heteroaryl is thiazolyl. In one embodiment, heteroaryl is imidazolyl. In one embodiment, heteroaryl is pyrazolyl. In one embodiment, heteroaryl is isoxazolyl. In one embodiment, heteroaryl is isothiazolyl. In one embodiment, heteroaryl is pyridyl. In one embodiment, heteroaryl is pyrazinyl. In one embodiment, heteroaryl is pyrimidinyl. In one embodiment, heteroaryl is pyridazinyl. In one embodiment, heteroaryl is quinolinyl. In one embodiment, heteroaryl is isoquinolinyl. In one embodiment, heteroaryl is cinnolinyl. In one embodiment, heteroaryl is quinazolinyl. In one embodiment, heteroaryl is quinoxalinyl. In one embodiment, heteroaryl is phthalazinyl. In one embodiment, heteroaryl is pteridinyl. In one embodiment, heteroaryl is benzofuranyl. In one embodiment, heteroaryl is dibenzofuranyl. In one embodiment, heteroaryl is benzothiophenyl. In one embodiment, heteroaryl is benzoxazolyl. In one embodiment, heteroaryl is benzthiazoyl. In one embodiment, heteroaryl is dibenzothiophenyl. In one embodiment, heteroaryl is indolyl. In one embodiment, heteroaryl is indolinyl. In one embodiment, heteroaryl is benzimidazolyl. In one embodiment, heteroaryl is indazolyl. In one embodiment, heteroaryl is benztriazolyl. In one embodiment, a heterocycle comprising nitrogen is aziridinyl. In one embodiment, a hetercycle comprising nitrogen is azetidinyl. In one embodiment, a heterocycle comprising nitrogen is pyrrolidinyl. In one embodiment, a heterocycle comprising nitrogen is piperidinyl. In one embodiment, a heterocycle comprising nitrogen is azepanyl. In one embodiment, a heterocycle comprising nitrogen is azocanyl. In one embodiment, acyl is —C(O)R^{3c}, and R^{3c} is alkyl. In one embodiment, acyl is —C(O)R^{3c}, and R^{3c} is alkenyl. In one embodiment, acyl is —C(O)R^{3c}, and R^{3c} is alkynyl. In one embodiment, acyl is —C(O)R^{3c}, and R^{3c} is cycloalkyl. In one embodiment, acyl is —C(O)R^{3c}, and R^{3c} is aryl. In one embodiment, acyl is —C(O)R^{3c}, and R^{3c} is heteroaryl.

In any preceding embodiment in this section, R⁷ is —O— or —NR^{7a}R^{7b}, wherein R^{7a} and R^{7b} are independently in each instance, a bond, hydrogen, alkyl, alkenyl, alkynyl, cycloalkyl, aryl, heteroaryl, acyl, a first N-terminal amino acid residue, or a first N-terminal peptide residue, wherein alkyl, alkenyl, alkynyl, cycloalkyl, aryl, heteroaryl, and acyl are optionally substituted. In certain embodiments R^{7a} is hydrogen and R^{7b} is a bond. In certain embodiments R⁷ is —O—. In certain embodiments R^{7a} is hydrogen and R^{7b} is a first N-terminal amino acid residue.

### Conjugates/Antibody Drug Conjugates (ADCs)

Provided herein are antibodies, or an antigen binding fragment thereof, wherein said antibody is conjugated to one or more compounds of Formula I, Ia, II, III, IV, V, or VI as described herein.

Provided herein are conjugates having a Formula A, B, C, D, or E

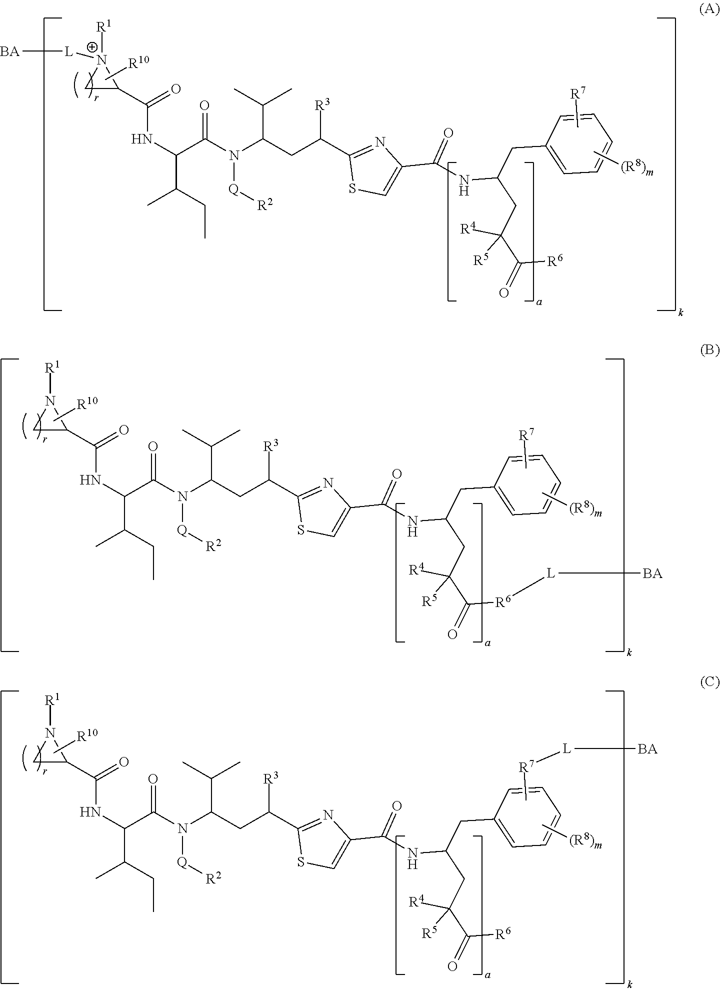

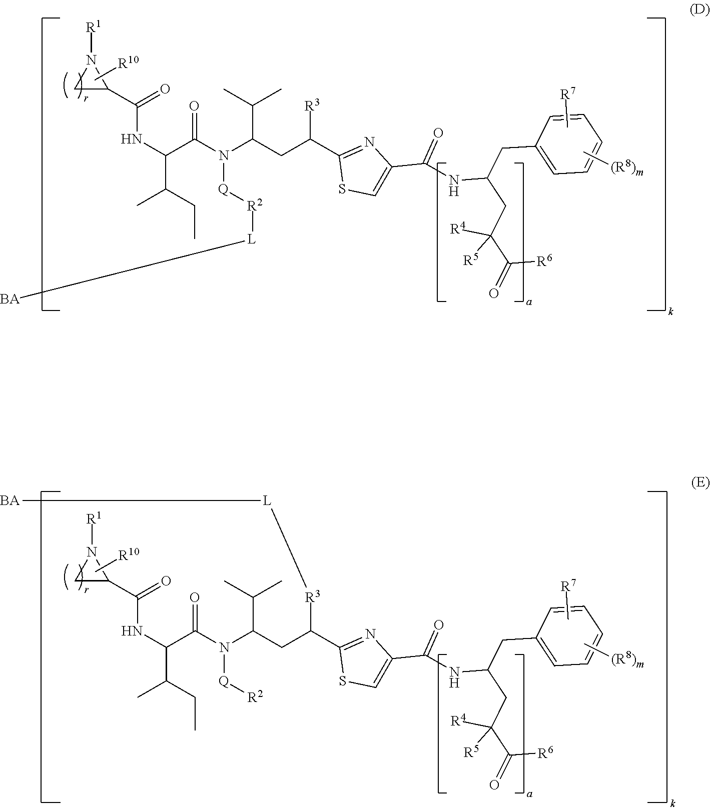

wherein L is a linker. In certain embodiments, R¹, Q, R², R³, R⁴, R⁵, R⁶, R⁷, R⁸, R¹⁰, m, r, and a are as described above in the context of Formula I, and k is 1, 2, 3, 4, 5, 6, 7, 8, 9, or 10. In certain embodiments, k is a range from 1-2, 1-3, 2-3, 2-4, 3-4, or 1-4.

Provided herein are conjugates of Formula

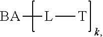

A, B, C, D, or E, wherein T is described elsewhere herein, or a pharmaceutically acceptable salt, solvate, regioisomeric, or stereoisomeric form thereof, wherein R⁷ is, independently in each instance, hydrogen, —OH, —O—, halogen, or —NR^{7a}R^{7b},

wherein R^{7a} and R^{7b} are, independently in each instance, a bond, hydrogen, alkyl, alkenyl, alkynyl, cycloalkyl, aryl, heteroaryl, acyl, —C(O)CH₂OH, —C(O)CH₂O—, a first N-terminal amino acid residue, a first N-terminal peptide residue, —CH₂CH₂NH₂, and —CH₂CH₂NH—, wherein alkyl, alkenyl, alkynyl, cycloalkyl, aryl, heteroaryl, and acyl are optionally substituted. In certain embodiments, R¹, Q, R², R³, R⁴, R⁵, R⁶, R⁷, R, R¹⁰, m, r, and a are as described above in the context of Formula I, and k is 1, 2, 3, 4, 5, 6, 7, 8, 9, or 10. In certain embodiments, k is a range from 1-2, 1-3, 2-3, 2-4, 3-4, or 1-4.

Provided herein are conjugates of A′, B′, C′, D′, or E′

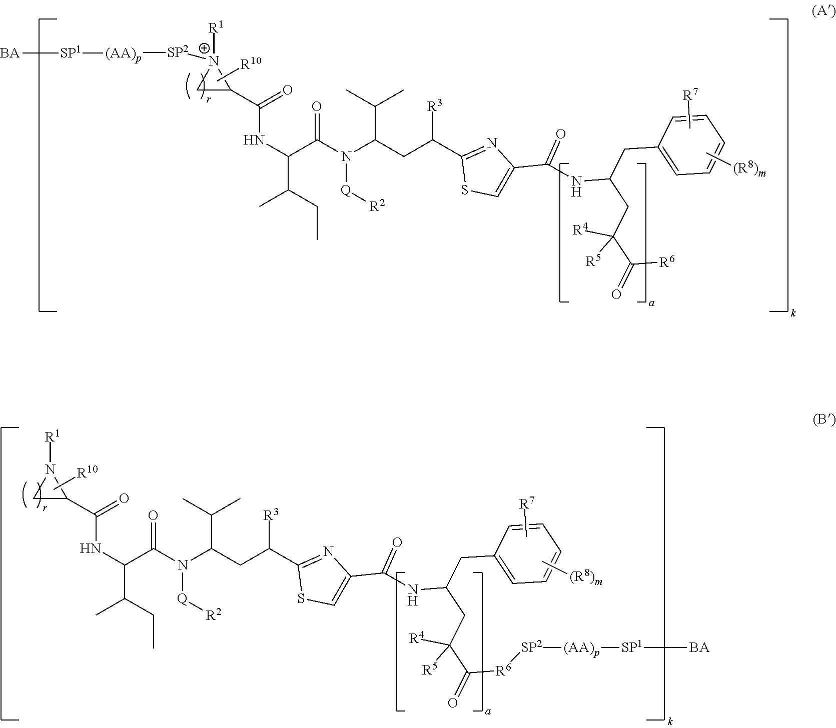

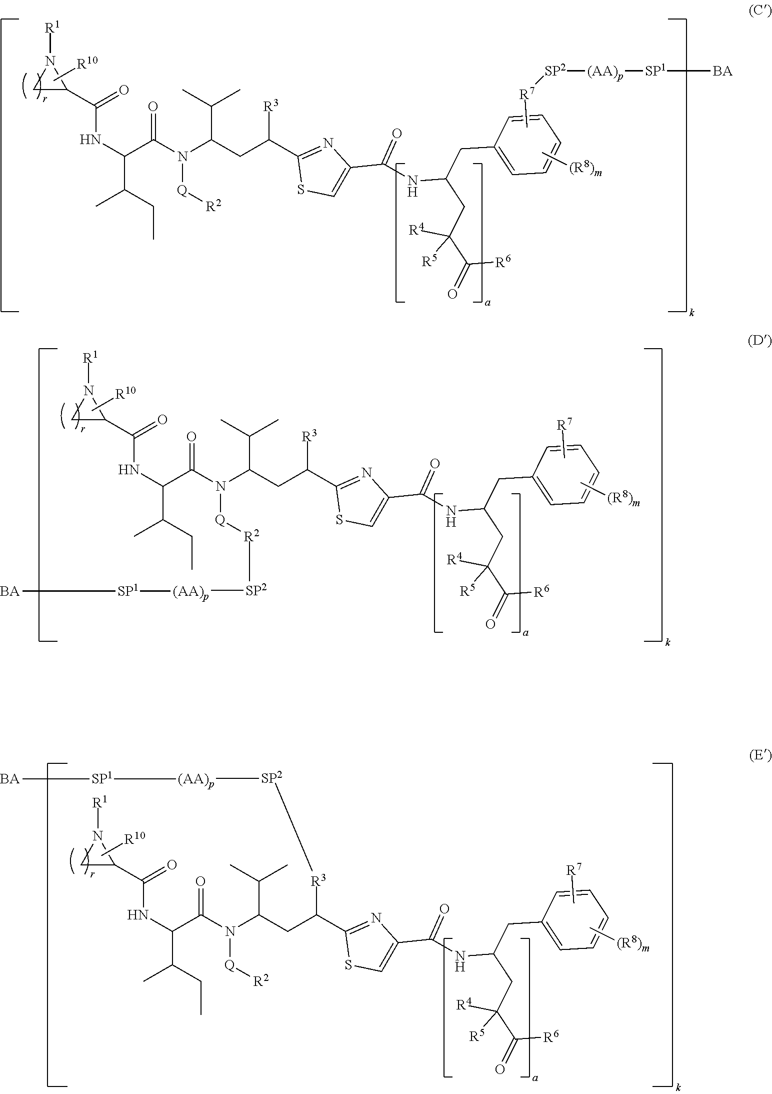

or a pharmaceutically acceptable salt, prodrug, solvate, regioisomeric, or stereoisomeric form thereof, wherein SP¹ and SP², when present, are spacer groups; each AA, when present, is a second amino acid residue; and p is an integer from zero to ten. In certain embodiments, R¹, Q, R², R³, R⁴, R⁵, R⁶, R⁷, R⁸, R¹⁰, m, r, and a are as described above in the context of Formula I, and k is 1, 2, 3, 4, 5, 6, 7, 8, 9, or 10. In certain embodiments, k is a range from 1-2, 1-3, 2-3, 2-4, 3-4, or 1-4. In certain embodiments, the -SP²- spacer, when present, is

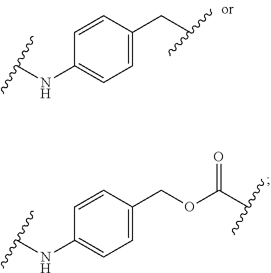 [SMILES: *Cc1ccc(N*)cc1 ; *Nc1ccc(COC(*)=O)cc1]

the second -(AA)ₚ- is

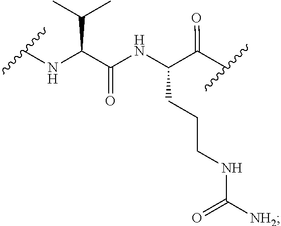 [SMILES: *N[C@H](C(=O)N[C@@H](CCCNC(N)=O)C(*)=O)C(C)C]

the —SP¹-spacer is

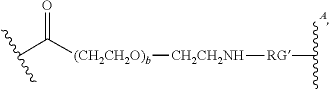

wherein RG′ is a reactive group residue following reaction of a reactive group RG with a binding agent; [symbol] is a bond, direct or indirect, to the binding agent; and b is an integer from one to four. In certain embodiments, p is as described above. In certain embodiments, b is one. In certain embodiments, b is two. In certain embodiments, b is three. In certain embodiments, b is four. In certain embodiments, Q is —O—. In certain embodiments, the conjugate has a structure of Formula A′, B′, C′, D′, or E′, wherein Q is —CH₂—; R¹ is C₁-C₁₀ alkyl; R² is alkyl; R⁴ and R⁵ are C₁-C₅ alkyl; R⁶ is —OH; R¹⁰ is absent; wherein r is four; and wherein a is one. In one embodiment, the conjugate has a structure of Formula C′, or a pharmaceutically acceptable salt thereof. In one embodiment, the conjugate has a structure of Formula C′, or a pharmaceutically acceptable salt thereof, wherein R⁷ is —NH—; and R⁸ is hydrogen or fluoro. In one embodiment, the conjugate has a structure of Formula C′, or a pharmaceutically acceptable salt thereof, wherein R⁷ is —NH—; and R⁸ is hydrogen. In one embodiment, the conjugate has a structure of Formula C′, or a pharmaceutically acceptable salt thereof, wherein R⁷ is —NH—; and R⁸ is fluoro. In one embodiment, the conjugate has a structure of Formula E′, or a pharmaceutically acceptable salt thereof. In one embodiment, the conjugate has a structure of Formula E′, or a pharmaceutically acceptable salt thereof, wherein R³ is —OC(O)N(H)CH₂CH₂NH—or —OC(O)N(H)CH₂CH₂OCH₂CH₂OCH₂CH₂OCH₂CH₂NH—. In one embodiment, the conjugate has a structure of Formula E′, or a pharmaceutically acceptable salt thereof, wherein R³ is —OC(O)N(H)CH₂CH₂NH—. In one embodiment, the conjugate has a structure of Formula E′, or a pharmaceutically acceptable salt thereof, wherein R³ is —OC(O)N(H)CH₂CH₂OCH₂CH₂OCH₂CH₂OCH₂CH₂NH—. In certain embodiments, the conjugate has a structure of Formula A′, B′, C′, D′, or E′, wherein Q is —CH₂—; R is hydrogen or C₁-C₁₀ alkyl; R² is alkyl; R⁴ and R⁵ are C₁-C₅ alkyl; R⁶ is —OH; wherein r is three or four; and wherein a is one. In one embodiment, the conjugate has a structure of Formula C′, or a pharmaceutically acceptable salt thereof. In one embodiment, the conjugate has a structure of Formula C′, or a pharmaceutically acceptable salt thereof, wherein R⁷ is —NH—; and R⁸ is hydrogen. In certain embodiments, the conjugate has a structure of Formula A′, B′, C′, D′, or E′, wherein Q is —CH₂—; R¹ is hydrogen or C₁-C₁₀ alkyl; R² is alkyl; R⁴ and R⁵ are C₁-C₅ alkyl; R⁶ is —OH; R⁰ is absent; wherein r is four; and wherein a is one. In one embodiment, the conjugate has a structure of Formula C′, or a pharmaceutically acceptable salt thereof. In one embodiment, the conjugate has a structure of Formula C′, or a pharmaceutically acceptable salt thereof, wherein R⁷ is —NH—; and R⁸ is hydrogen. In certain embodiments, the conjugate has a structure of Formula A′, B′, C′, D′, or E′, wherein Q is —O—; R¹ is hydrogen or C₁-C₁₀ alkyl; R² is alkyl or alkynyl; R³ is hydroxyl or —OC(O)C₁-C₅ alkyl; R⁴ and R⁵ are C₁-C₅ alkyl; R⁶ is —OH; R⁰, when present, is —C₁-C₅ alkyl; wherein r is three or four; and wherein a is one. In one embodiment, the conjugate has a structure of Formula C′, or a pharmaceutically acceptable salt thereof. In one embodiment, the conjugate has a structure of Formula C′, or a pharmaceutically acceptable salt thereof, R⁷ is —NH—; and R⁸ is hydrogen. In certain embodiments, the conjugate has a structure of Formula A′, B′, C′, D′, or E′, wherein Q is —CH₂— or —O—; R¹ is C₁-C₁₀ alkyl; R² is alkyl or alkynyl; R⁴ and R⁵ are C₁-C₅ alkyl; R⁶ is —NHSO₂(CH₂)ₐ₁-aryl-(CH₂)ₐ₂NR^{6a}R^{6b};
R¹⁰ is absent; wherein r is four; and wherein a, a1, and, a2 are, independently, zero or one. In one embodiment, the conjugate has a structure of Formula B′, or a pharmaceutically acceptable salt thereof. In one embodiment, the conjugate has a structure of Formula B′, or a pharmaceutically acceptable salt thereof, wherein R⁶ is

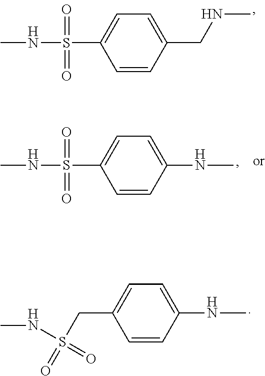

In one embodiment, the conjugate has a structure of Formula B′, or a pharmaceutically acceptable salt thereof, wherein R⁶ is

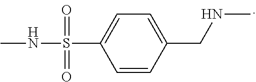

In one embodiment, the conjugate has a structure of Formula B′, or a pharmaceutically acceptable salt thereof, wherein R⁶ is

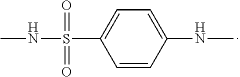

In one embodiment, the conjugate has a structure of Formula B′, or a pharmaceutically acceptable salt thereof, wherein R⁶ is

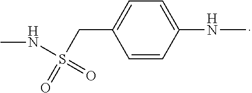

In one embodiment, the conjugate has a structure of Formula B′, or a pharmaceutically acceptable salt thereof, wherein a is zero; and R⁶ is

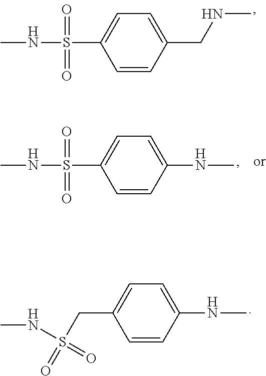

In one embodiment, the conjugate has a structure of Formula B′, or a pharmaceutically acceptable salt thereof, wherein a is zero; and R⁶ is

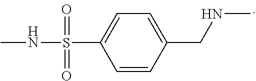

In one embodiment, the conjugate has a structure of Formula B′, or a pharmaceutically acceptable salt thereof, wherein a is zero; and R⁶ is

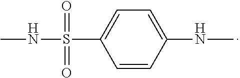

In one embodiment, the conjugate has a structure of Formula B′, or a pharmaceutically acceptable salt thereof, wherein a is zero; and R⁶ is

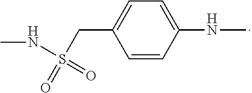

In one embodiment, the conjugate has a structure of Formula B′, or a pharmaceutically acceptable salt thereof, wherein a is one; and R⁶ is

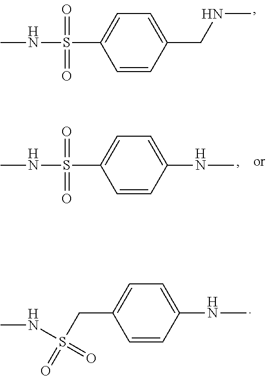

In one embodiment, the conjugate has a structure of Formula B′, or a pharmaceutically acceptable salt thereof, wherein a is one; and R⁶ is

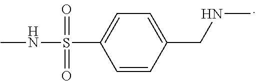

In one embodiment, the conjugate has a structure of Formula B′, or a pharmaceutically acceptable salt thereof, wherein a is one; and R⁶ is

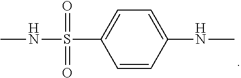

In one embodiment, the conjugate has a structure of Formula B′, or a pharmaceutically acceptable salt thereof, wherein a is one; and R⁶ is

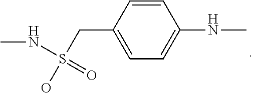

In one embodiment, the conjugate has a structure of Formula C′, or a pharmaceutically acceptable salt thereof, wherein R⁷ is —O—; and R¹ is hydrogen.

Provided herein are conjugates of Formula A, In certain embodiments, compounds conjugated to -L-BA in Formula A include one or more compounds of Formulae I, Ia, II, III, IV, V, and/or VI as described above, wherein BA is a binding agent; L is a linker; and k is 1, 2, 3, 4, 5, 6, 7, 8, 9, or 10. In certain embodiments, k is a range from 1-2, 1-3, 2-3, 2-4, 3-4, or 1-4. In any embodiment in this paragraph, BA is antibody, or antigen binding fragment thereof, wherein the antibody is conjugated to a compound of Formula I, as described above. In any embodiment in this paragraph, BA is antibody, or antigen binding fragment thereof, wherein the antibody is conjugated to a compound of Formula Ia, as described above. In any embodiment in this paragraph, BA is antibody, or antigen binding fragment thereof, wherein the antibody is conjugated to a compound of Formula II, as described above. In any embodiment in this paragraph, BA is antibody, or antigen binding fragment thereof, wherein the antibody is conjugated to a compound of Formula III, as described above. In any embodiment in this paragraph, BA is antibody, or antigen binding fragment thereof, wherein the antibody is conjugated to a compound of Formula IV, as described above. In any embodiment in this paragraph, BA is antibody, or antigen binding fragment thereof, wherein the antibody is conjugated to a compound of Formula V, as described above. In any embodiment in this paragraph, BA is antibody, or antigen binding fragment thereof, wherein the antibody is conjugated to a compound of Formula VI, as described above. In any of the embodiments in this paragraph, any one or more compounds of Formulae I, Ia, II, III, IV, V, and/or VI conjugated to -L-BA in Formula A are conjugated via the heterocycle comprising nitrogen, as described elsewhere herein. In certain embodiments, when Q is —O—, then R² is C₁-C₁₀ alkyl, C₁-C₁₀ alkynyl, a regioisomeric triazole, —C₁-C₁₀ alkylene-(5-membered heteroaryl), —C₁-C₃ alkylene-Q-(CH₂)ₙₙaryl, C₁-C₃ hydroxyalkyl, or C₁-C₁₀ alkylether. In certain embodiments in this paragraph, nn is one. In certain embodiments in this paragraph, nn is two. In certain embodiments in this paragraph, nn is three. In certain embodiments in this paragraph, nn is four. In certain embodiments in this paragraph, nn is five. In certain embodiments in this paragraph, nn is six. In certain embodiments in this paragraph, nn is seven. In certain embodiments in this paragraph, nn is eight. In certain embodiments in this paragraph, nn is nine. In certain embodiments in this paragraph, nn is ten. In certain embodiments in this paragraph, Qi is —CH₂—. In certain embodiments in this paragraph, Qi is —O—. In certain embodiments, when Q is —CH₂—, then R² is C₅-C₁₀ alkyl, C₁-C₁₀ alkynyl, —C₁-C₁₀ alkylene-(5-membered heteroaryl), —C₁-C₃ alkylene-Q¹-(CH₂)ₙₙaryl, C₁-C₃ hydroxyalkyl, or C₁-C₁₀ alkylether, In certain embodiments in this paragraph, nn is one. In certain embodiments in this paragraph, nn is two. In certain embodiments in this paragraph, nn is three. In certain embodiments in this paragraph, nn is four. In certain embodiments in this paragraph, nn is five. In certain embodiments in this paragraph, nn is six. In certain embodiments in this paragraph, nn is seven. In certain embodiments in this paragraph, nn is eight. In certain embodiments in this paragraph, nn is nine. In certain embodiments in this paragraph, nn is ten. In certain embodiments in this paragraph, Q¹ is —CH₂—. In certain embodiments in this paragraph, Q¹ is —O—.

Provided herein are conjugates of Formula B. In certain embodiments, compounds conjugated to -L-BA in Formula B include one or more compounds of Formulae I, Ia, II, III, IV, V, and/or VI, as described above, wherein BA is a binding agent; L is a linker; and k is 1, 2, 3, 4, 5, 6, 7, 8, 9, or 10. In certain embodiments, k is a range from 1-2, 1-3, 2-3, 2-4, 3-4, or 1-4. In any embodiment in this paragraph, BA is antibody, or antigen binding fragment thereof, wherein the antibody is conjugated to a compound of Formula I, as described above. In any embodiment in this paragraph, BA is antibody, or antigen binding fragment thereof, wherein the antibody is conjugated to a compound of Formula Ia, as described above. In any embodiment in this paragraph, BA is antibody, or antigen binding fragment thereof, wherein the antibody is conjugated to a compound of Formula II, as described above. In any embodiment in this paragraph, BA is antibody, or antigen binding fragment thereof, wherein the antibody is conjugated to a compound of Formula III, as described above. In any embodiment in this paragraph, BA is antibody, or antigen binding fragment thereof, wherein the antibody is conjugated to a compound of Formula IV, as described above. In any embodiment in this paragraph, BA is antibody, or antigen binding fragment thereof, wherein the antibody is conjugated to a compound of Formula V, as described above. In any embodiment in this paragraph, BA is antibody, or antigen binding fragment thereof, wherein the antibody is conjugated to a compound of Formula VI, as described above. In any of the embodiments in this paragraph, any one or more compounds of Formulae I, Ia, II, III, IV, V, and/or VI conjugated to -L -BA in Formula B are conjugated via divalent R⁶. In certain embodiments, when Q is —O—, then R² is C₁-C₁₀ alkyl, C₁-C₁₀ alkynyl, a regioisomeric triazole, —C₁-C₁₀ alkylene-(5-membered heteroaryl), —C₁-C₃ alkylene-Q¹-(CH₂)ₙₙaryl, C₁-C₃ hydroxyalkyl, or C₁-C₁₀ alkylether. In certain embodiments in this paragraph, nn is one. In certain embodiments in this paragraph, nn is two. In certain embodiments in this paragraph, nn is three. In certain embodiments in this paragraph, nn is four. In certain embodiments in this paragraph, nn is five. In certain embodiments in this paragraph, nn is six. In certain embodiments in this paragraph, nn is seven. In certain embodiments in this paragraph, nn is eight. In certain embodiments in this paragraph, nn is nine. In certain embodiments in this paragraph, nn is ten. In certain embodiments in this paragraph, Q¹ is —CH₂—. In certain embodiments in this paragraph, Q¹ is —O—. In certain embodiments, when Q is —CH₂—, then R² is C₅-C₁₀ alkyl, C₁-C₁₀ alkynyl, —C₁-C₁₀ alkylene-(5-membered heteroaryl), —C₁-C₃ alkylene-Qi-(CH₂)ₙₙaryl, C₁-C₃ hydroxyalkyl, or C₁-C₁₀ alkylether, In certain embodiments in this paragraph, nn is one. In certain embodiments in this paragraph, nn is two. In certain embodiments in this paragraph, nn is three. In certain embodiments in this paragraph, nn is four. In certain embodiments in this paragraph, nn is five. In certain embodiments in this paragraph, nn is six. In certain embodiments in this paragraph, nn is seven. In certain embodiments in this paragraph, nn is eight. In certain embodiments in this paragraph, nn is nine. In certain embodiments in this paragraph, nn is ten. In certain embodiments in this paragraph, Qi is —CH₂—. In certain embodiments in this paragraph, Qi is —O—.

Provided herein are conjugates of Formula C. In certain embodiments, compounds conjugated to -L-BA in Formula C include one or more compounds of Formulae I, Ia, II, III, IV, V, and/or VI as described above, wherein BA is a binding agent; L is a linker; and k is 1, 2, 3, 4, 5, 6, 7, 8, 9, or 10. In certain embodiments, k is a range from 1-2, 1-3, 2-3, 2-4, 3-4, or 1-4. In any embodiment in this paragraph, BA is antibody, or antigen binding fragment thereof, wherein the antibody is conjugated to a compound of Formula I, as described above. In any embodiment in this paragraph, BA is antibody, or antigen binding fragment thereof, wherein the antibody is conjugated to a compound of Formula Ia, as described above. In any embodiment in this paragraph, BA is antibody, or antigen binding fragment thereof, wherein the antibody is conjugated to a compound of Formula II, as described above. In any embodiment in this paragraph, BA is antibody, or antigen binding fragment thereof, wherein the antibody is conjugated to a compound of Formula III, as described above. In any embodiment in this paragraph, BA is antibody, or antigen binding fragment thereof, wherein the antibody is conjugated to a compound of Formula IV, as described above. In any embodiment in this paragraph, BA is antibody, or antigen binding fragment thereof, wherein the antibody is conjugated to a compound of Formula V, as described above. In any embodiment in this paragraph, BA is antibody, or antigen binding fragment thereof, wherein the antibody is conjugated to a compound of Formula VI, as described above. In any of the embodiments in this paragraph, any one or more compounds of Formulae I, Ia, II, III, IV, V, and/or VI conjugated to -L-BA in Formula C are conjugated via divalent R⁷. In certain embodiments, when Q is —O—, then R² is C₁-C₁₀ alkyl, C₁-C₁₀ alkynyl, a regioisomeric triazole, —C₁-C₁₀ alkylene-(5-membered heteroaryl), —C₁-C₃ alkylene-Qi-(CH₂)ₙₙaryl, C₁-C₃ hydroxyalkyl, or C₁-C₁₀ alkylether. In certain embodiments in this paragraph, nn is one. In certain embodiments in this paragraph, nn is two. In certain embodiments in this paragraph, nn is three. In certain embodiments in this paragraph, nn is four. In certain embodiments in this paragraph, nn is five. In certain embodiments in this paragraph, nn is six. In certain embodiments in this paragraph, nn is seven. In certain embodiments in this paragraph, nn is eight. In certain embodiments in this paragraph, nn is nine. In certain embodiments in this paragraph, nn is ten. In certain embodiments in this paragraph, Qi is —CH₂—. In certain embodiments in this paragraph, Qi is —O—. In certain embodiments, when Q is —CH₂—, then R² is C₅-C₁₀ alkyl, C₁-C₁₀ alkynyl, —C₁-C₁₀ alkylene-(5-membered heteroaryl), —C₁-C₃ alkylene-Qi-(CH₂)ₙₙaryl, C₁-C₃ hydroxyalkyl, or C₁-C₁₀ alkylether, In certain embodiments in this paragraph, nn is one. In certain embodiments in this paragraph, nn is two. In certain embodiments in this paragraph, nn is three. In certain embodiments in this paragraph, nn is four. In certain embodiments in this paragraph, nn is five. In certain embodiments in this paragraph, nn is six. In certain embodiments in this paragraph, nn is seven. In certain embodiments in this paragraph, nn is eight. In certain embodiments in this paragraph, nn is nine. In certain embodiments in this paragraph, nn is ten. In certain embodiments in this paragraph, Q¹ is —CH₂—. In certain embodiments in this paragraph, Q¹ is —O—.

Provided herein are conjugates of Formula D. In certain embodiments, compounds conjugated to -L-BA in Formula D include one or more compounds of Formulae I, Ia, II, III, IV, V, and/or VI as described above, wherein BA is a binding agent; L is a linker; and k is 1, 2, 3, 4, 5, 6, 7, 8, 9, or 10. In certain embodiments, k is a range from 1-2, 1-3, 2-3, 2-4, 3-4, or 1-4. In any embodiment in this paragraph, BA is antibody, or antigen binding fragment thereof, wherein the antibody is conjugated to a compound of Formula I, as described above. In any embodiment in this paragraph, BA is antibody, or antigen binding fragment thereof, wherein the antibody is conjugated to a compound of Formula Ia, as described above. In any embodiment in this paragraph, BA is antibody, or antigen binding fragment thereof, wherein the antibody is conjugated to a compound of Formula II, as described above. In any embodiment in this paragraph, BA is antibody, or antigen binding fragment thereof, wherein the antibody is conjugated to a compound of Formula III, as described above. In any embodiment in this paragraph, BA is antibody, or antigen binding fragment thereof, wherein the antibody is conjugated to a compound of Formula IV, as described above. In any embodiment in this paragraph, BA is antibody, or antigen binding fragment thereof, wherein the antibody is conjugated to a compound of Formula V, as described above. In any embodiment in this paragraph, BA is antibody, or antigen binding fragment thereof, wherein the antibody is conjugated to a compound of Formula VI, as described above. In any of the embodiments in this paragraph, any one or more compounds of Formulae I, Ia, II, III, IV, V, and/or VI conjugated to -L -BA in Formula D are conjugated via divalent R². In certain embodiments, when Q is —O—, then R² is C₁-C₁₀ alkylene, C₁-C₁₀ alkynylene, a regioisomeric C₁-C₁₀ triazolylene, a regioisomeric —C₁-C₁₀ alkylene-(5-membered heteroarylene), or —C₁-C₃ alkylene-Q-(CH₂)ₙₙarylene. In certain embodiments in this paragraph, nn is one. In certain embodiments in this paragraph, nn is two. In certain embodiments in this paragraph, nn is three. In certain embodiments in this paragraph, nn is four. In certain embodiments in this paragraph, nn is five. In certain embodiments in this paragraph, nn is six. In certain embodiments in this paragraph, nn is seven. In certain embodiments in this paragraph, nn is eight. In certain embodiments in this paragraph, nn is nine. In certain embodiments in this paragraph, nn is ten. In certain embodiments in this paragraph, Q¹ is —CH₂—. In certain embodiments in this paragraph, Q¹ is —O—. In certain embodiments, when Q is —CH₂—, then R² is C₅-C₁₀ alkylene, C₁-C₁₀ alkynylene, a regioisomeric C₁-C₁₀ triazolylene, a regioisomeric —C₁-C₁₀ alkylene-(5-membered heteroarylene), or —C₁-C₃ alkylene-Q-(CH₂)ₙₙarylene. In certain embodiments in this paragraph, nn is one. In certain embodiments in this paragraph, nn is two. In certain embodiments in this paragraph, nn is three. In certain embodiments in this paragraph, nn is four. In certain embodiments in this paragraph, nn is five. In certain embodiments in this paragraph, nn is six. In certain embodiments in this paragraph, nn is seven. In certain embodiments in this paragraph, nn is eight. In certain embodiments in this paragraph, nn is nine. In certain embodiments in this paragraph, nn is ten. In certain embodiments in this paragraph, Q¹ is —CH₂—. In certain embodiments in this paragraph, Q¹ is —O—.

Provided herein are conjugates of Formula E. In certain embodiments, compounds conjugated to -L-BA in Formula E include one or more compounds of Formulae I, Ia, II, III, IV, V, and/or VI as described above, wherein BA is a binding agent; L is a linker; and k is 1, 2, 3, 4, 5, 6, 7, 8, 9, or 10. In certain embodiments, k is a range from 1-2, 1-3, 2-3, 2-4, 3-4, or 1-4. In any embodiment in this paragraph, BA is antibody, or antigen binding fragment thereof, wherein the antibody is conjugated to a compound of Formula I, as described above. In any embodiment in this paragraph, BA is antibody, or antigen binding fragment thereof, wherein the antibody is conjugated to a compound of Formula Ia, as described above. In any embodiment in this paragraph, BA is antibody, or antigen binding fragment thereof, wherein the antibody is conjugated to a compound of Formula II, as described above. In any embodiment in this paragraph, BA is antibody, or antigen binding fragment thereof, wherein the antibody is conjugated to a compound of Formula III, as described above. In any embodiment in this paragraph, BA is antibody, or antigen binding fragment thereof, wherein the antibody is conjugated to a compound of Formula IV, as described above. In any embodiment in this paragraph, BA is antibody, or antigen binding fragment thereof, wherein the antibody is conjugated to a compound of Formula V, as described above. In any embodiment in this paragraph, BA is antibody, or antigen binding fragment thereof, wherein the antibody is conjugated to a compound of Formula VI, as described above. In any of the embodiments in this paragraph, any one or more compounds of Formulae I, Ia, II, III, IV, V, and/or VI conjugated to -L-BA in Formula E are conjugated via divalent R³. In certain embodiments, when Q is —O—, then R² is C₁-C₁₀ alkyl, C₁-C₁₀ alkynyl, a regioisomeric triazole, —C₁-C₁₀ alkylene-(5-membered heteroaryl), —C₁-C₃ alkylene-Qi-(CH₂)ₙₙaryl, C₁-C₃ hydroxyalkyl, or C₁-C₁₀ alkylether. In certain embodiments in this paragraph, nn is one. In certain embodiments in this paragraph, nn is two. In certain embodiments in this paragraph, nn is three. In certain embodiments in this paragraph, nn is four. In certain embodiments in this paragraph, nn is five. In certain embodiments in this paragraph, nn is six. In certain embodiments in this paragraph, nn is seven. In certain embodiments in this paragraph, nn is eight. In certain embodiments in this paragraph, nn is nine. In certain embodiments in this paragraph, nn is ten. In certain embodiments in this paragraph, Qi is —CH₂—. In certain embodiments in this paragraph, Qi is —O—. In certain embodiments, when Q is —CH₂—, then R² is C₅-C₁₀ alkyl, C₁-C₁₀ alkynyl, —C₁-C₁₀ alkylene-(5-membered heteroaryl), —C₁-C₃ alkylene-Qi-(CH₂)ₙₙaryl, C₁-C₃ hydroxyalkyl, or C₁-C₁₀ alkylether, In certain embodiments in this paragraph, nn is one. In certain embodiments in this paragraph, nn is two. In certain embodiments in this paragraph, nn is three. In certain embodiments in this paragraph, nn is four. In certain embodiments in this paragraph, nn is five. In certain embodiments in this paragraph, nn is six. In certain embodiments in this paragraph, nn is seven. In certain embodiments in this paragraph, nn is eight. In certain embodiments in this paragraph, nn is nine. In certain embodiments in this paragraph, nn is ten. In certain embodiments in this paragraph, Q¹ is —CH₂—. In certain embodiments in this paragraph, Q¹ is —O—.

In certain embodiments, the compound of Formula A′, B′, C′, D′, or E′ is selected from the group consisting of

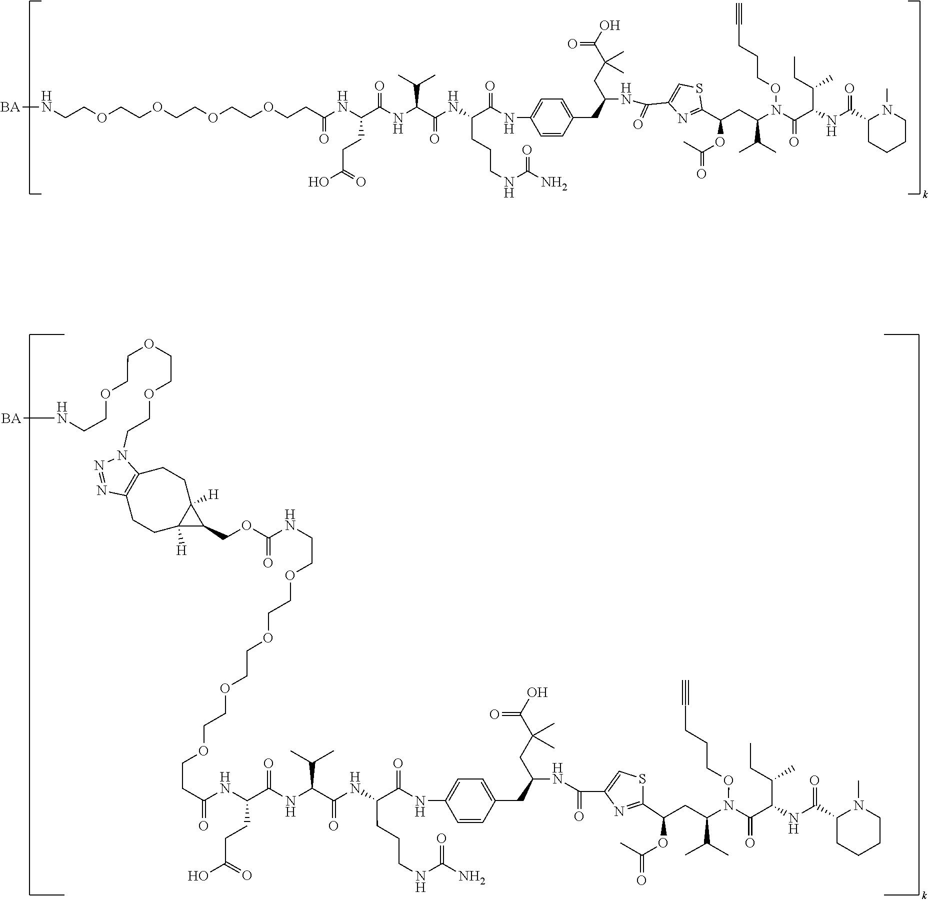

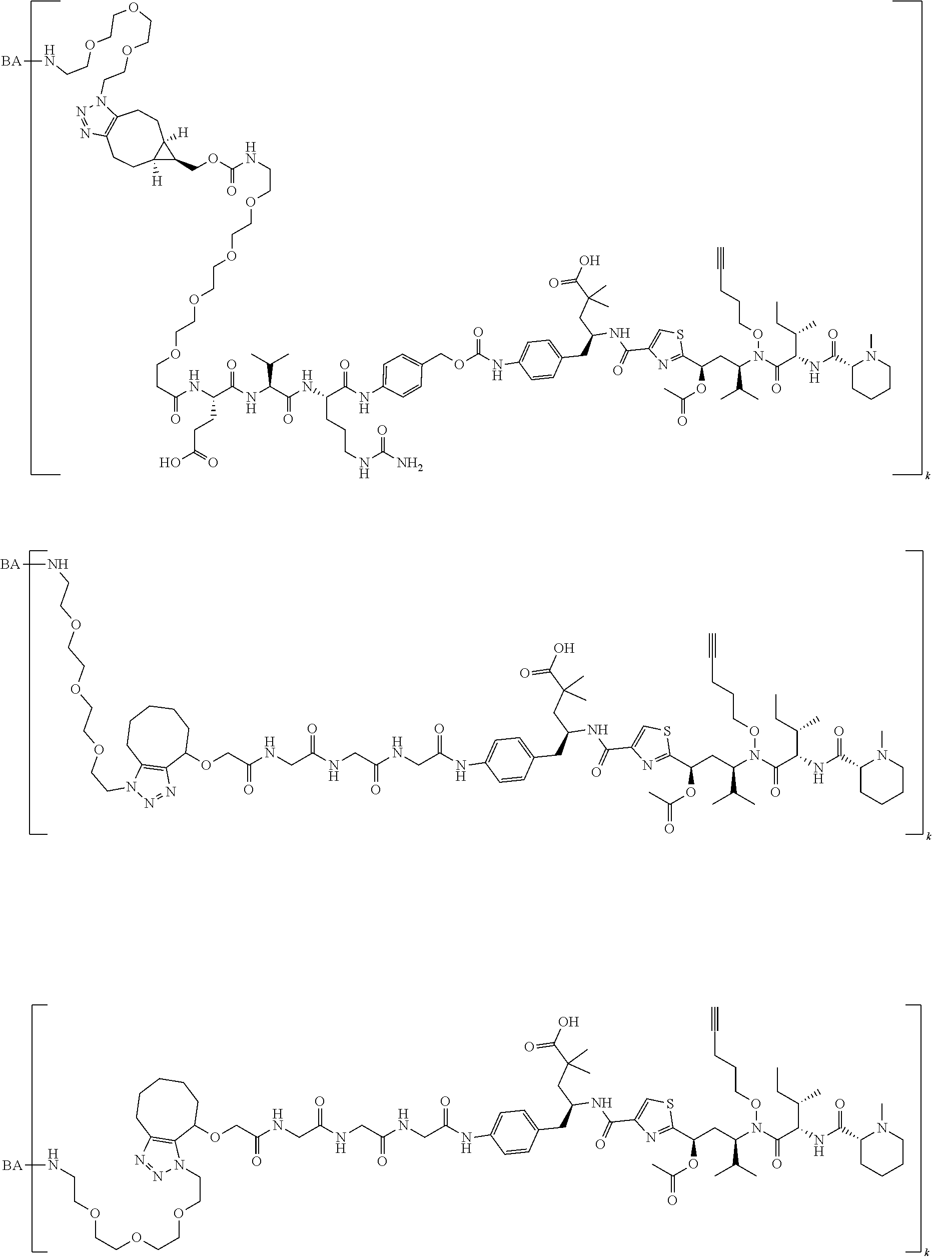

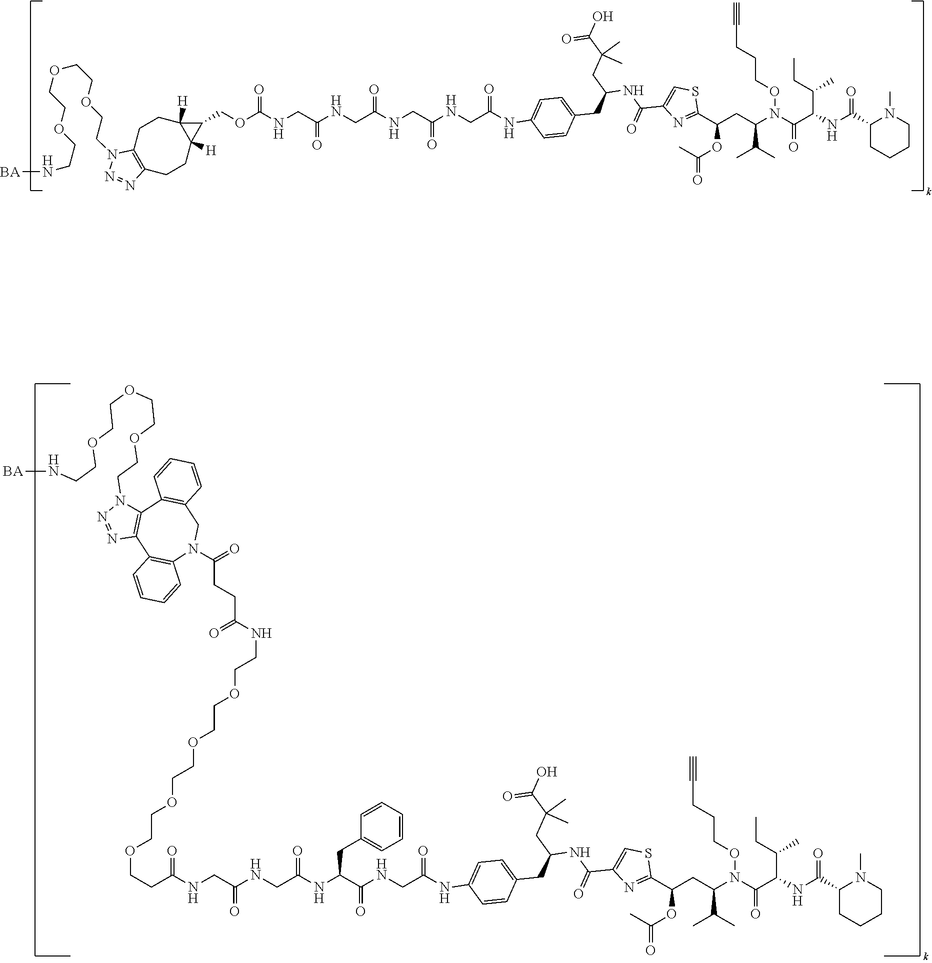

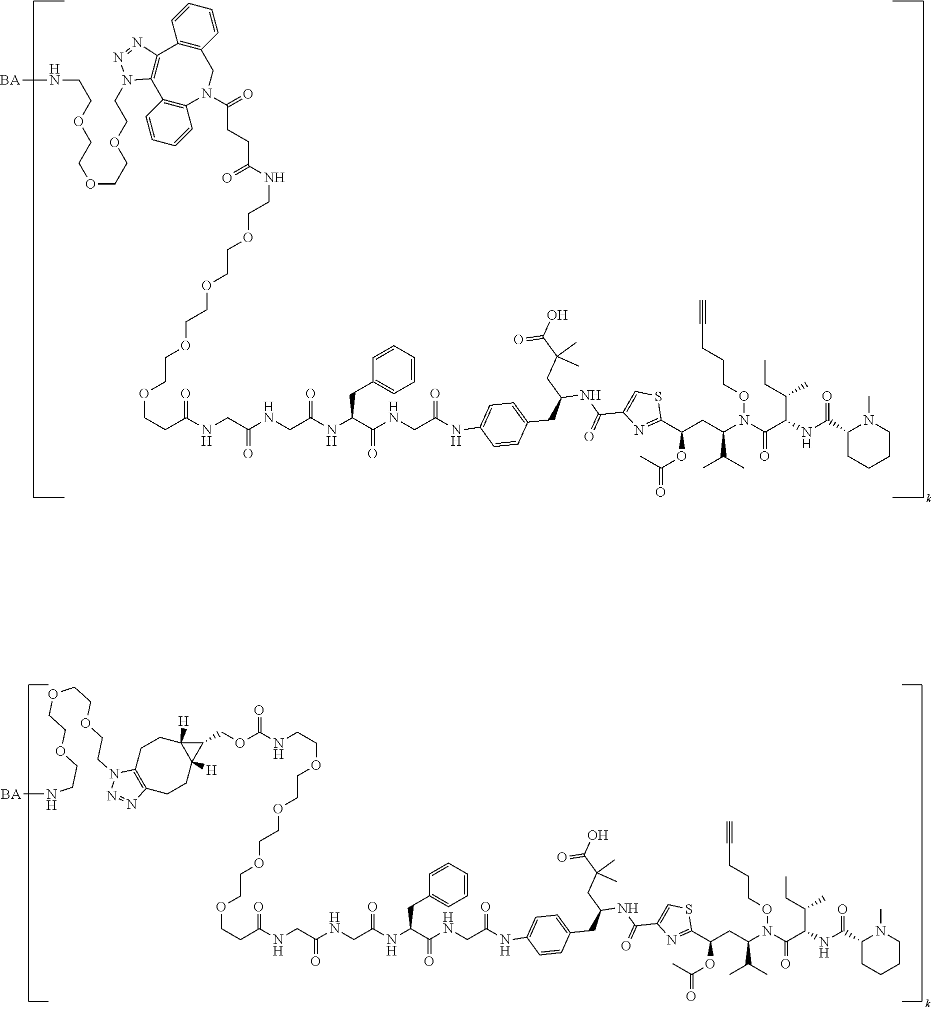

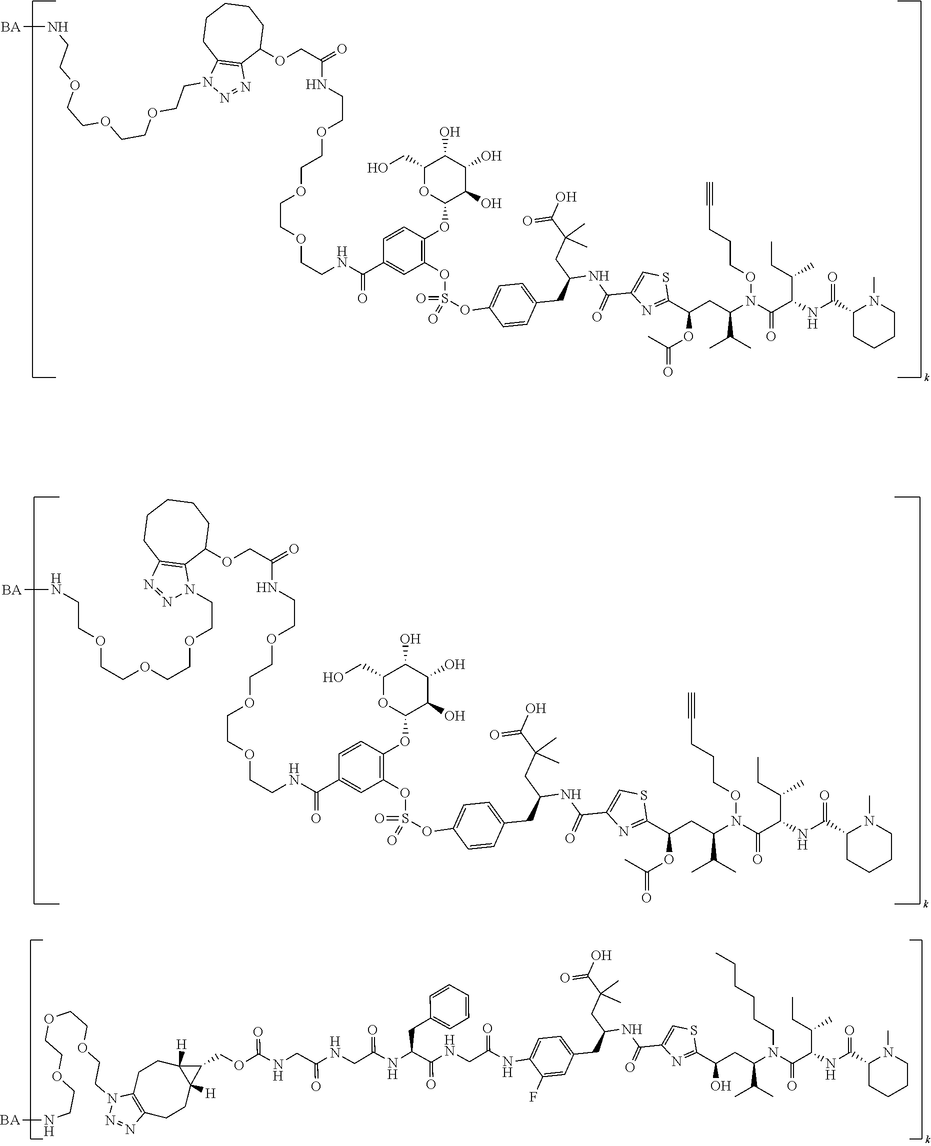

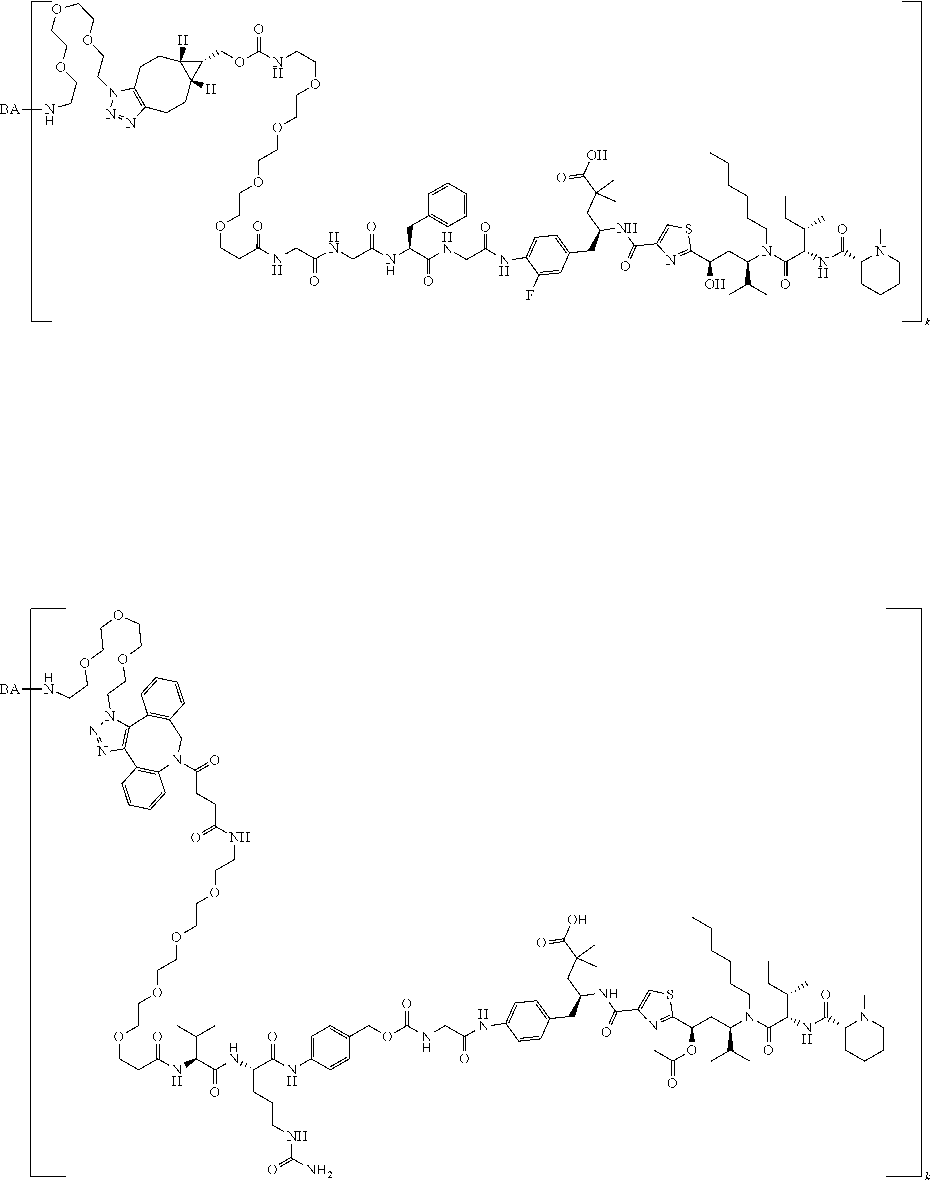

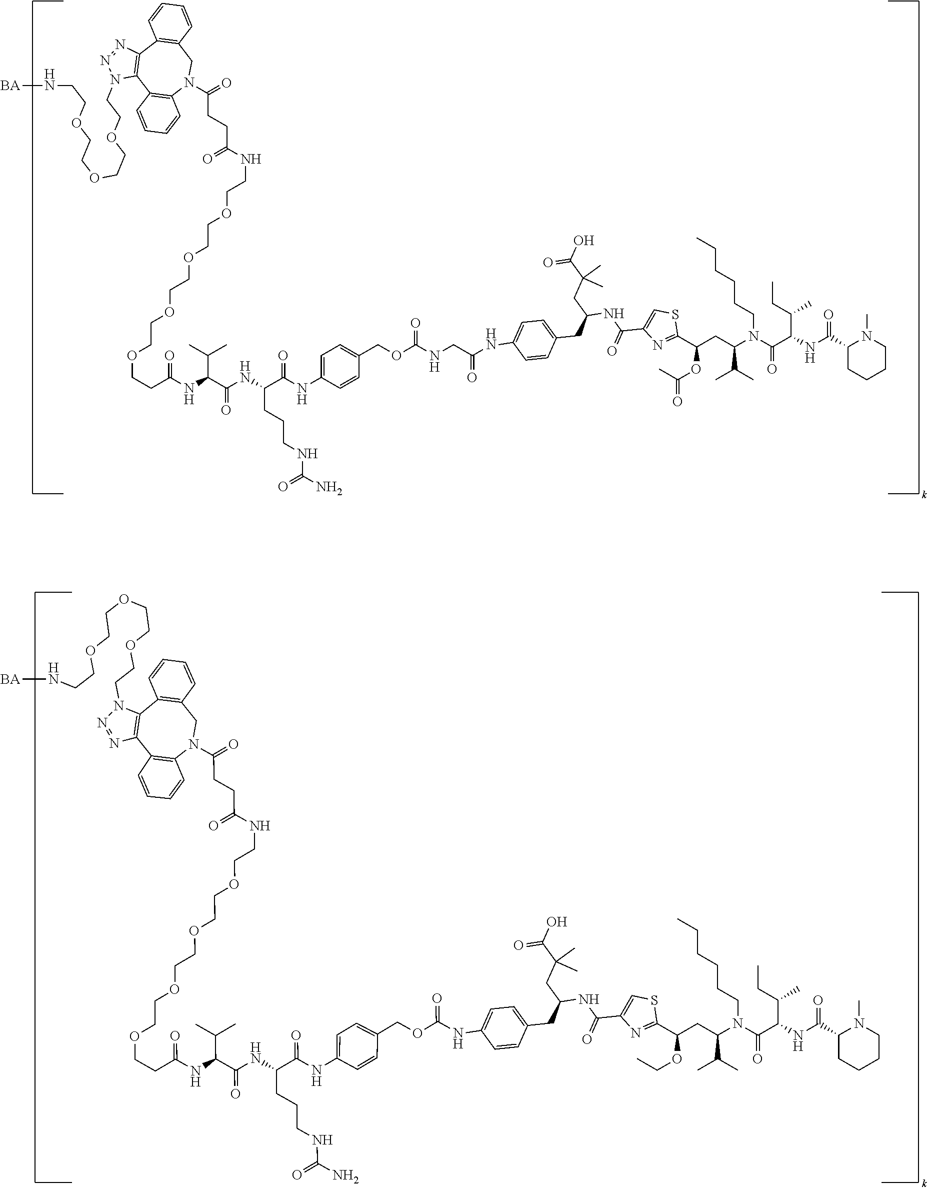

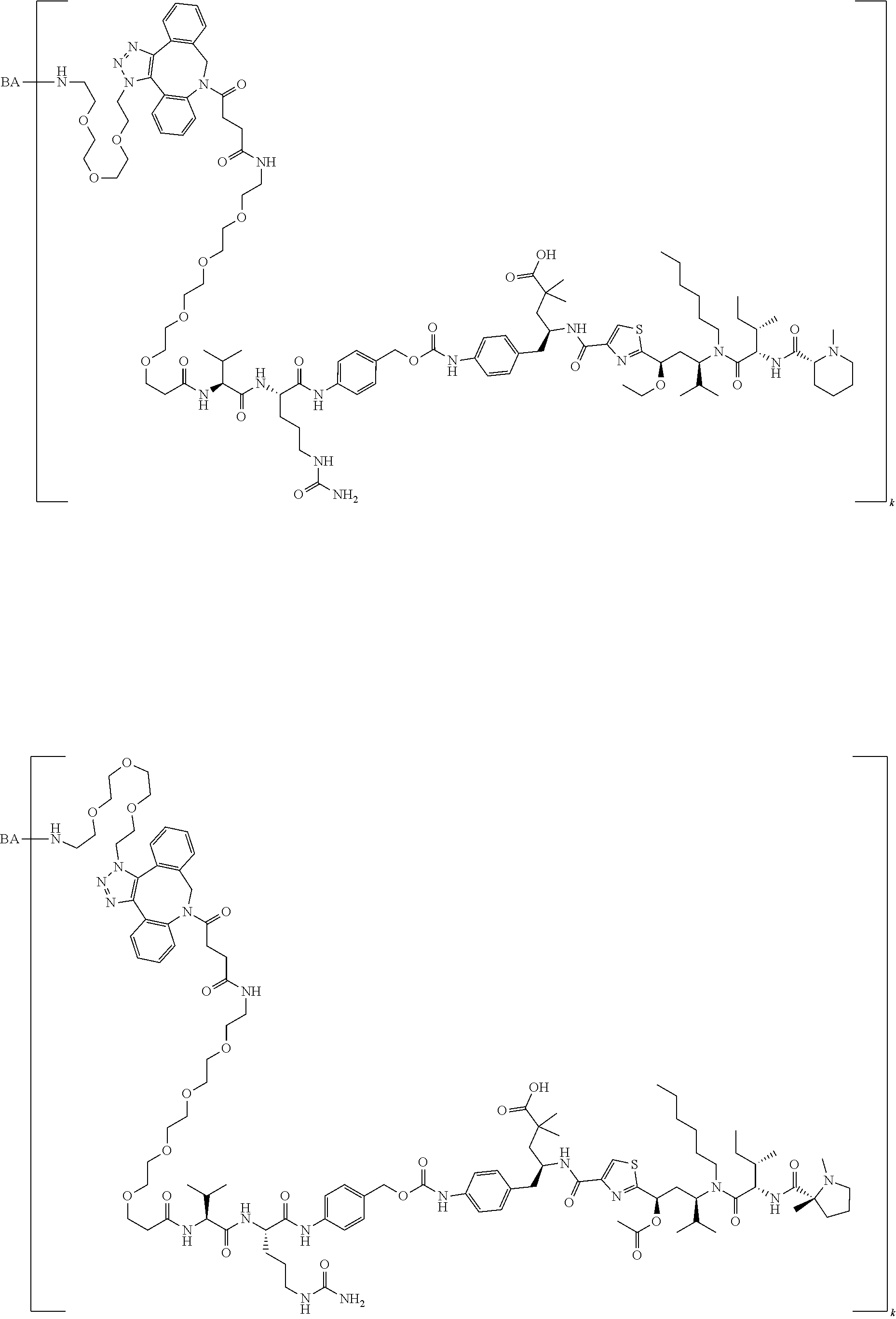

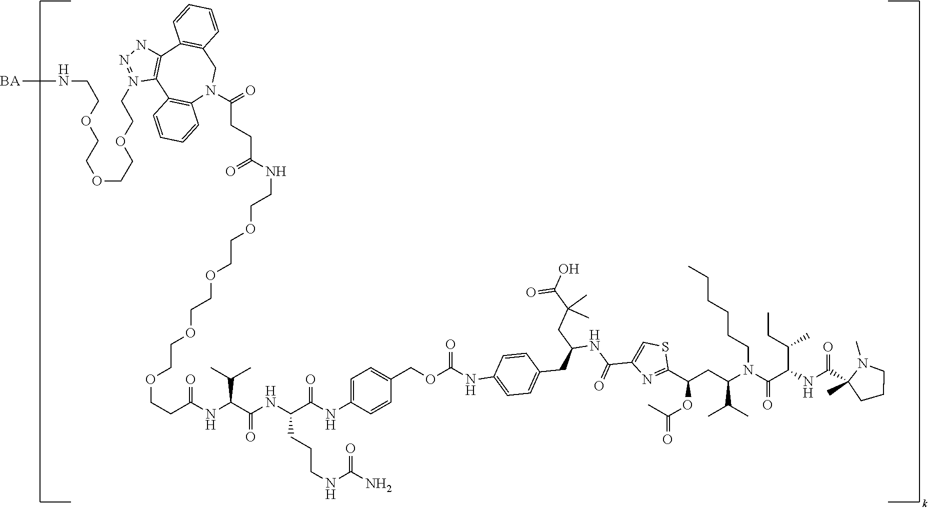

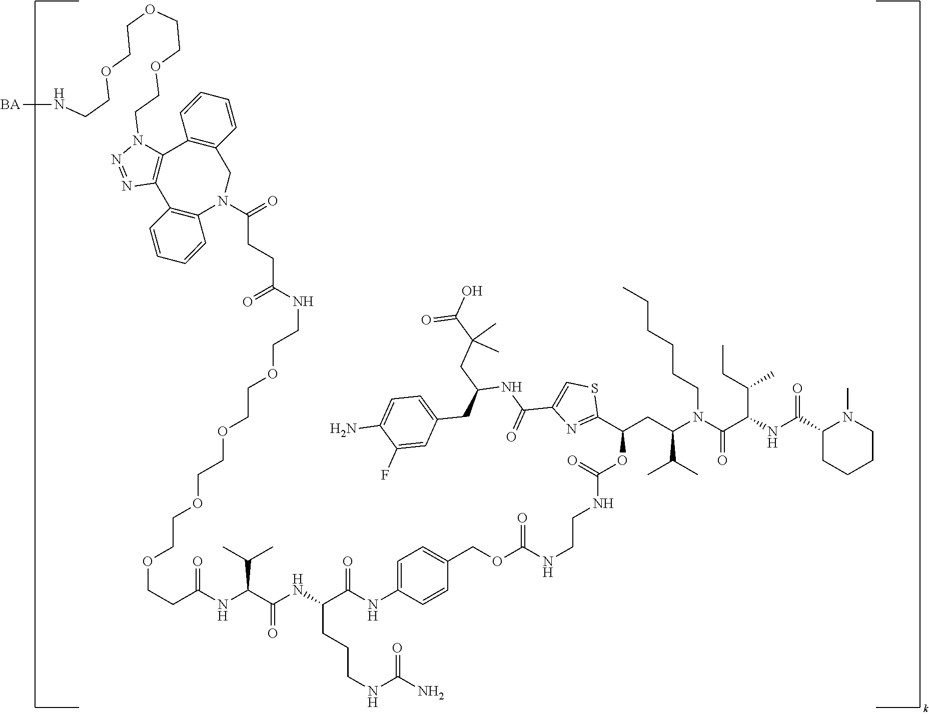

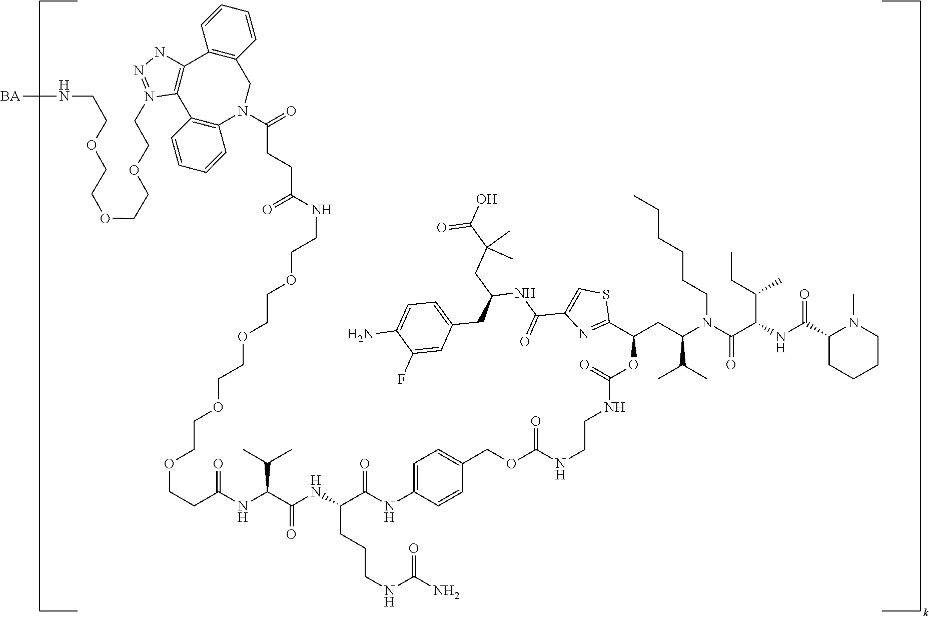

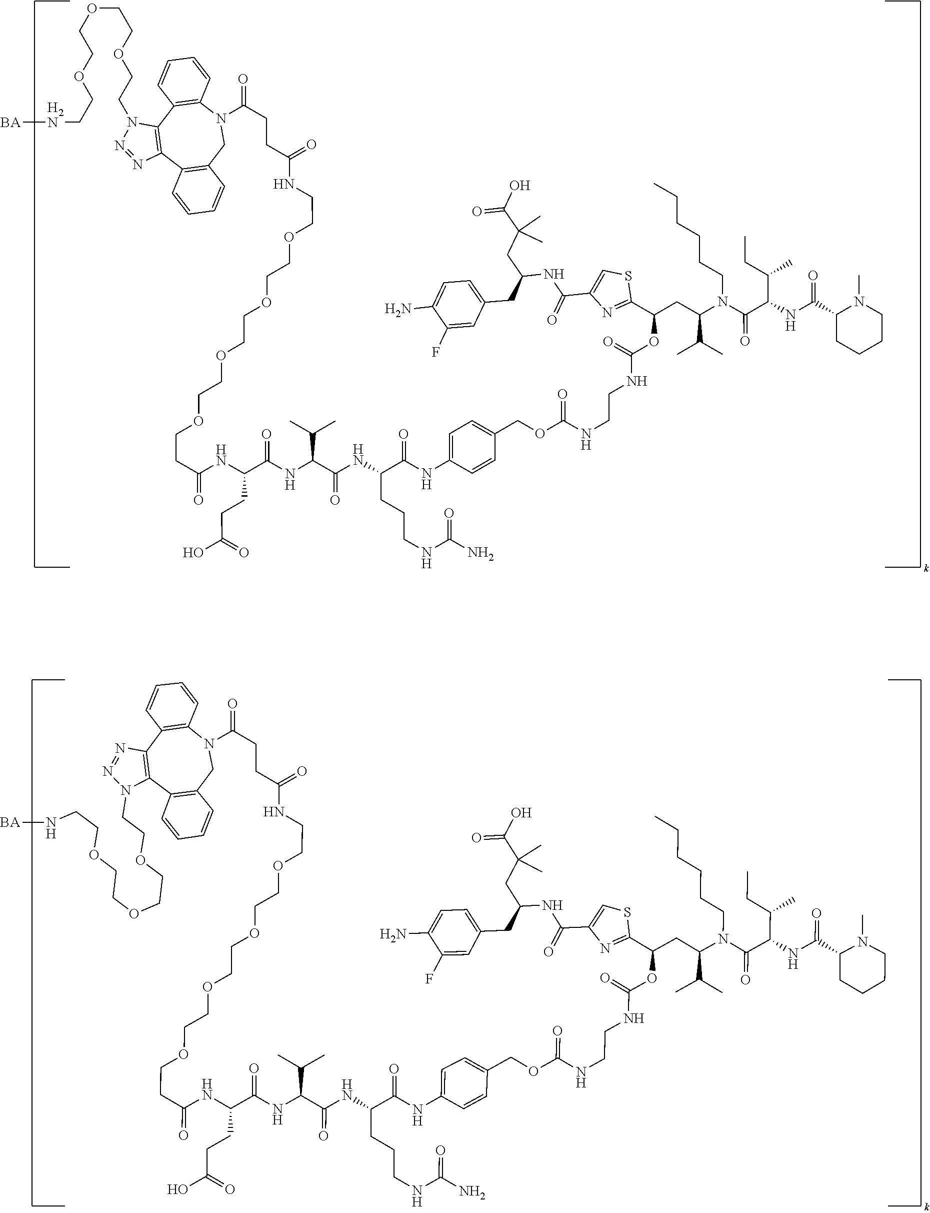

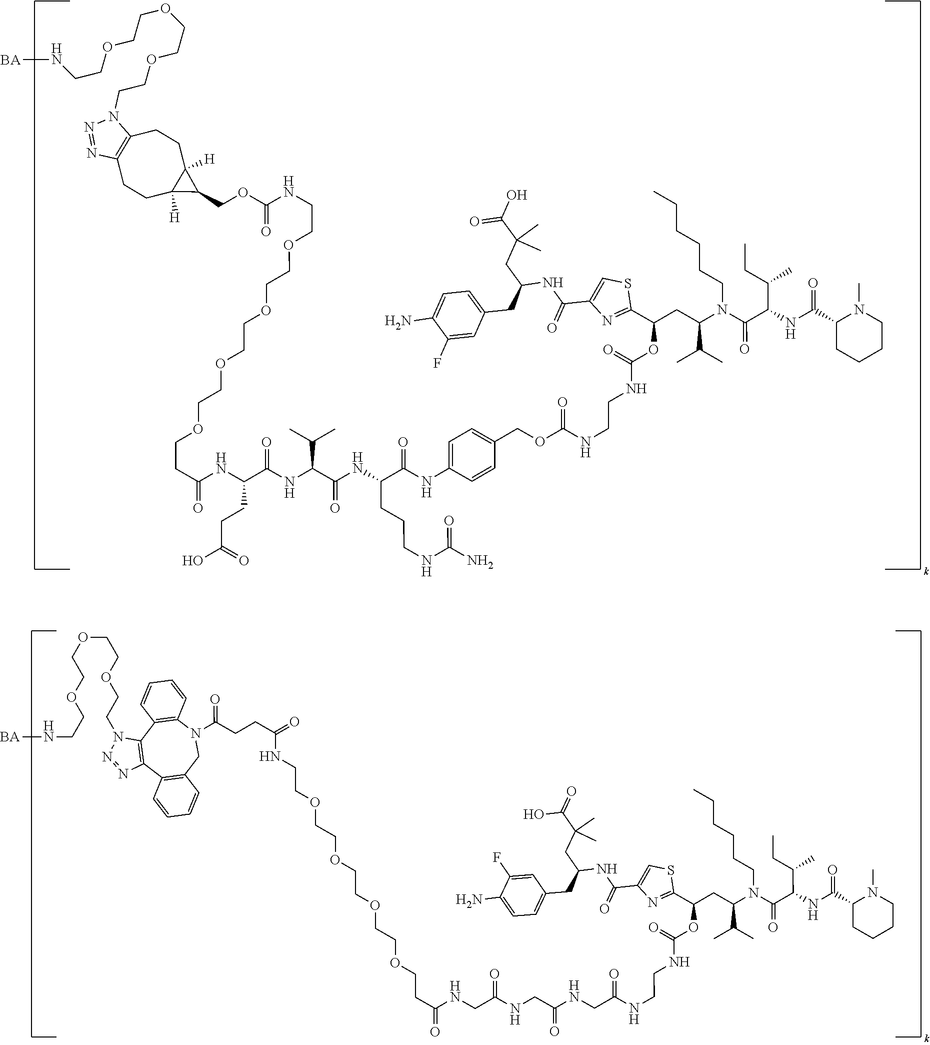

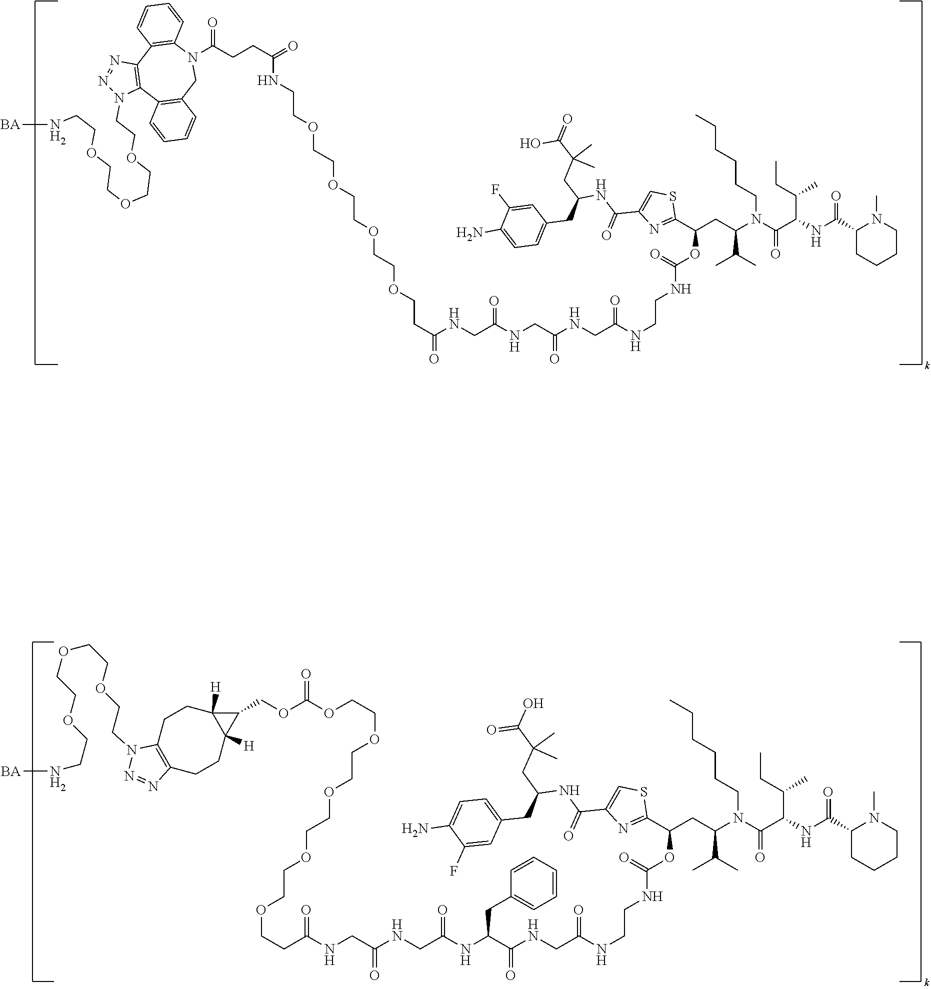

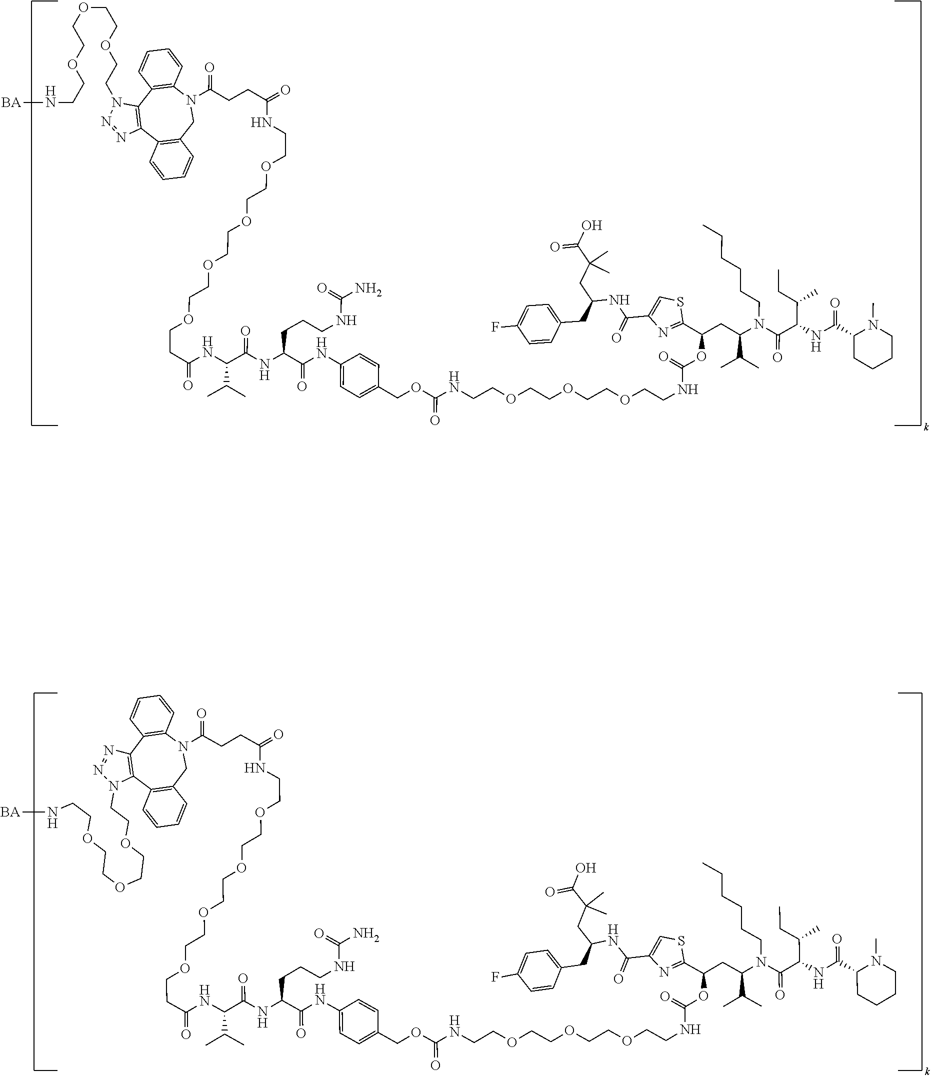

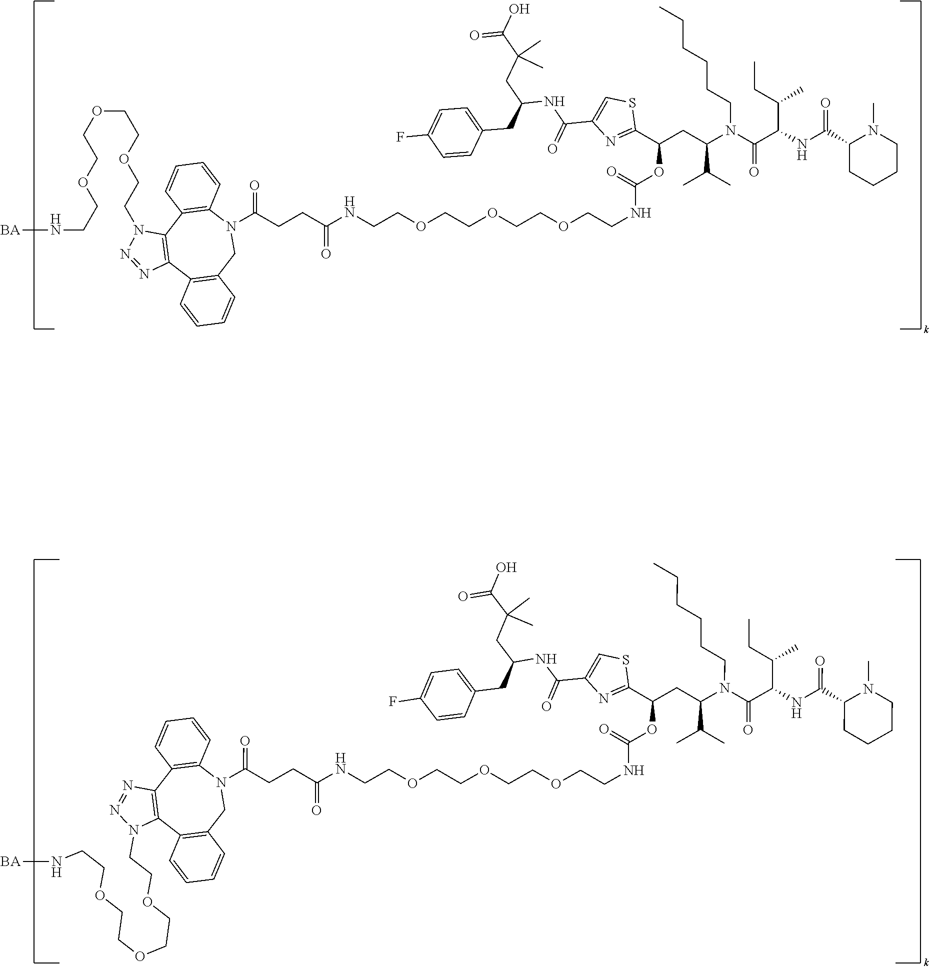

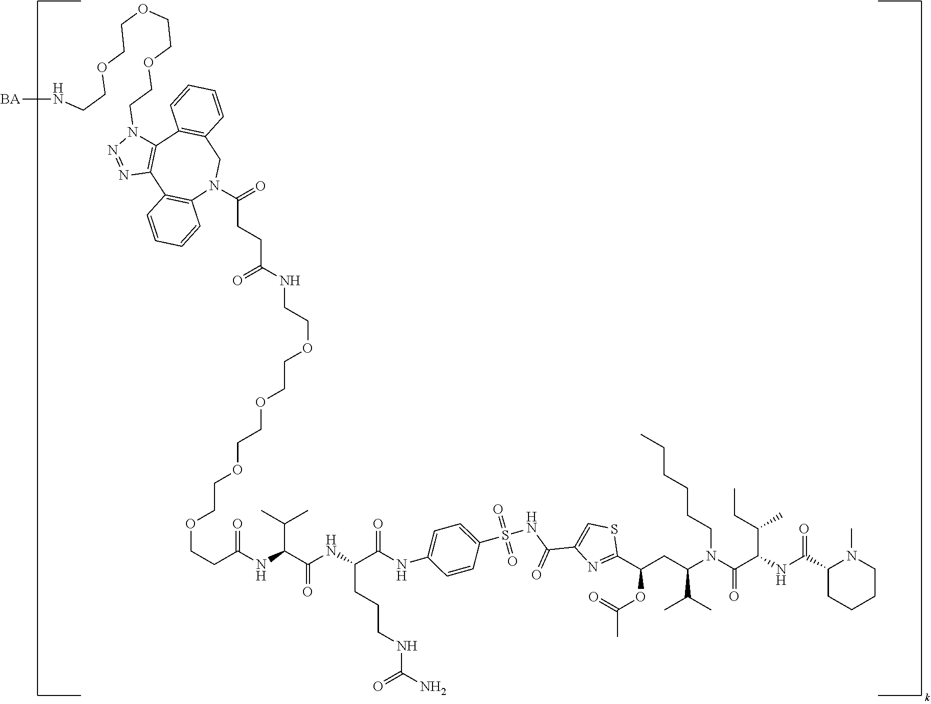

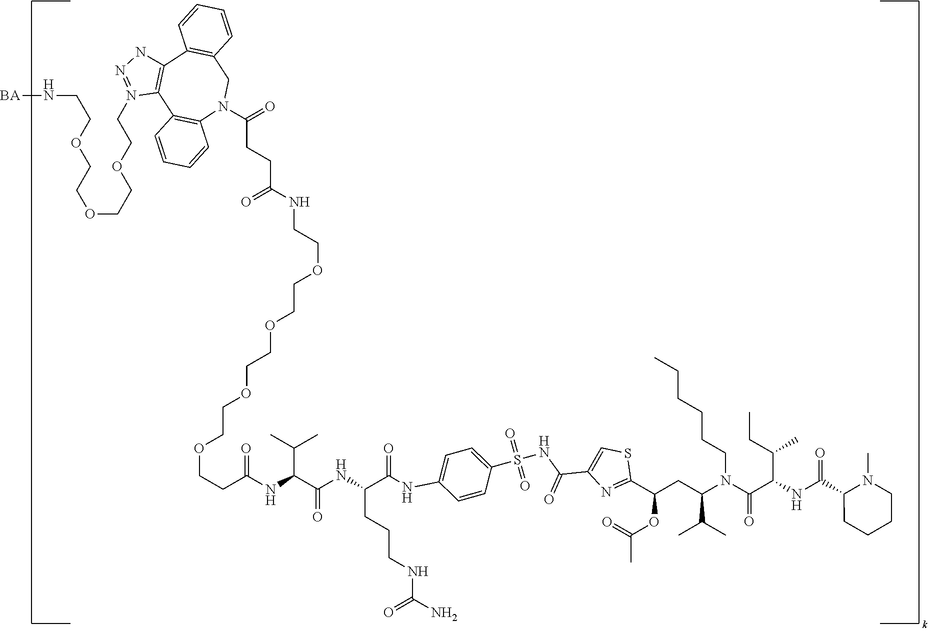

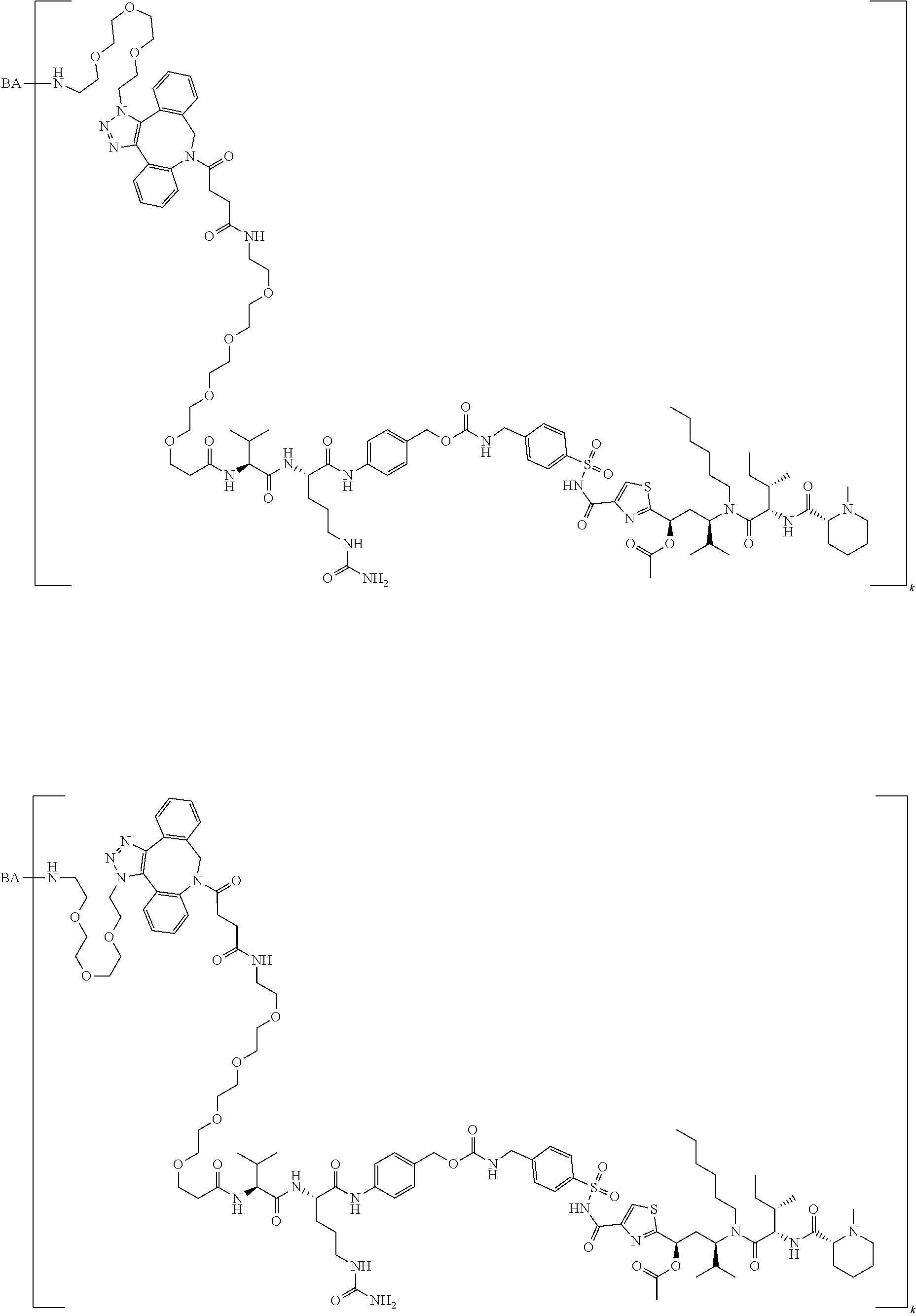

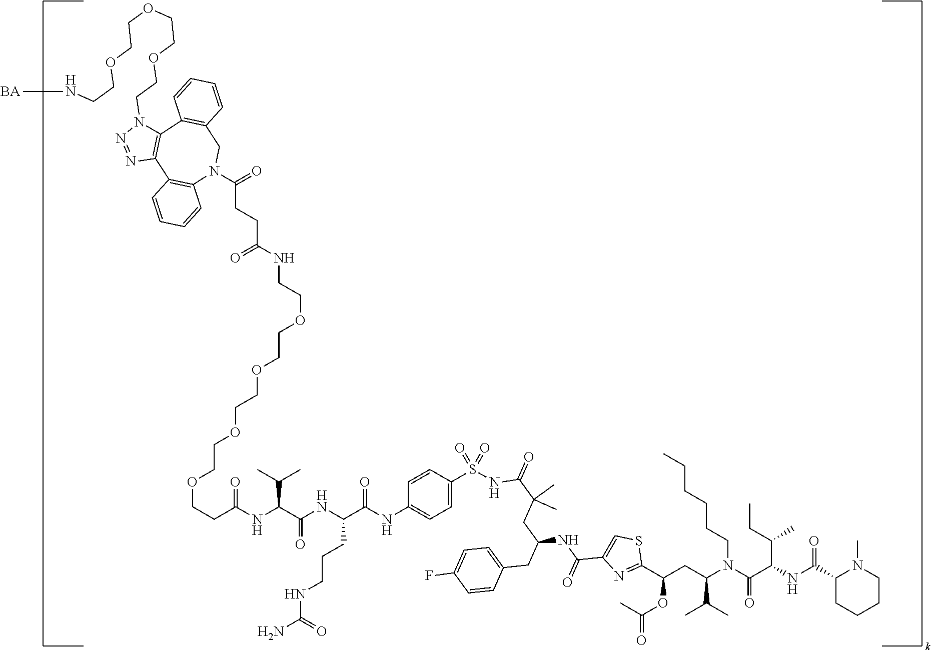

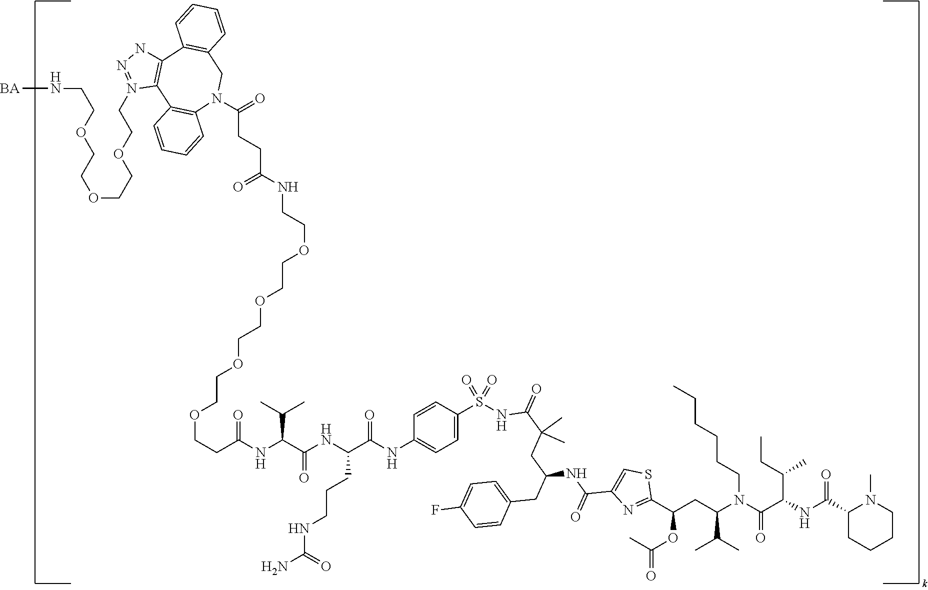

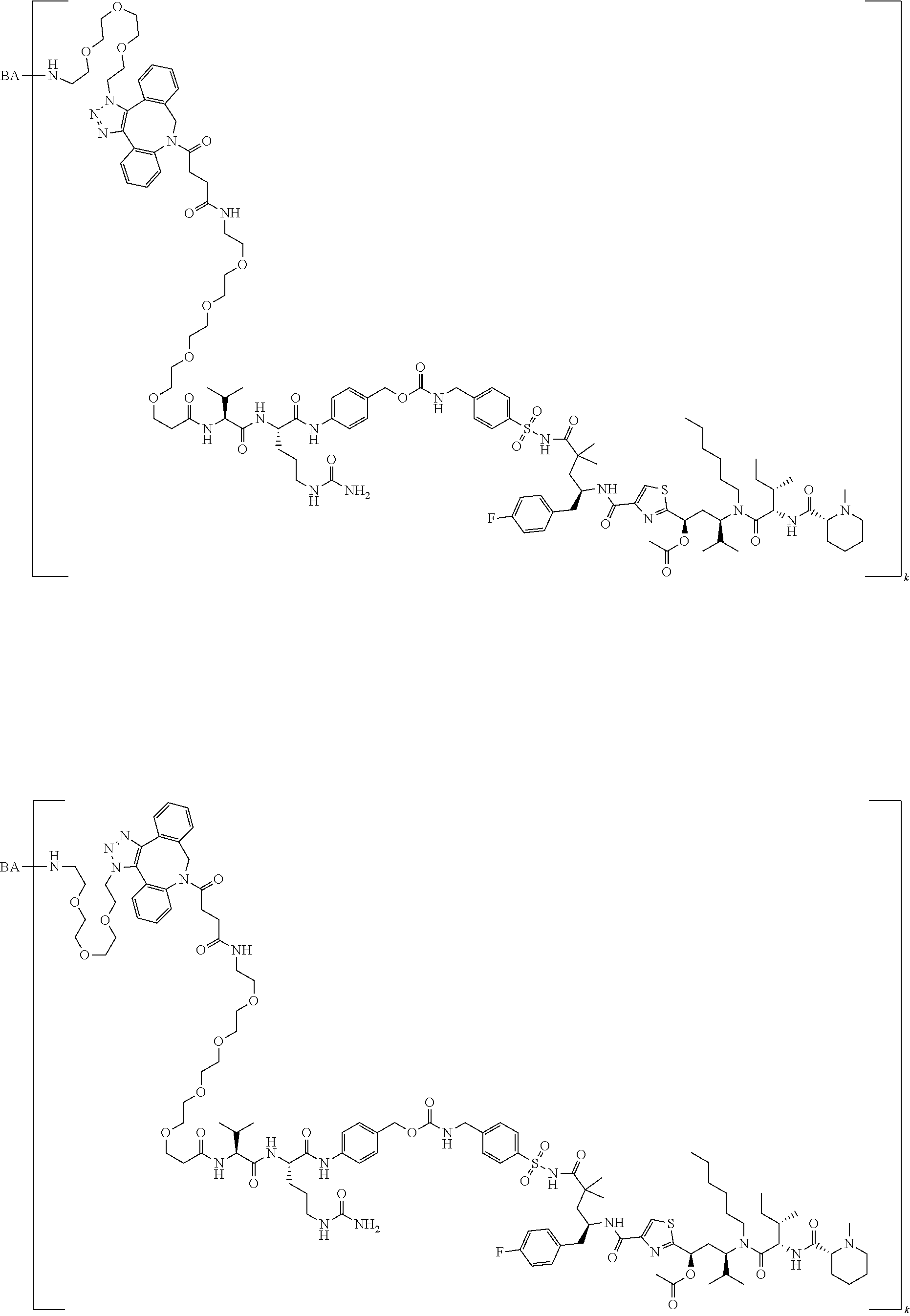

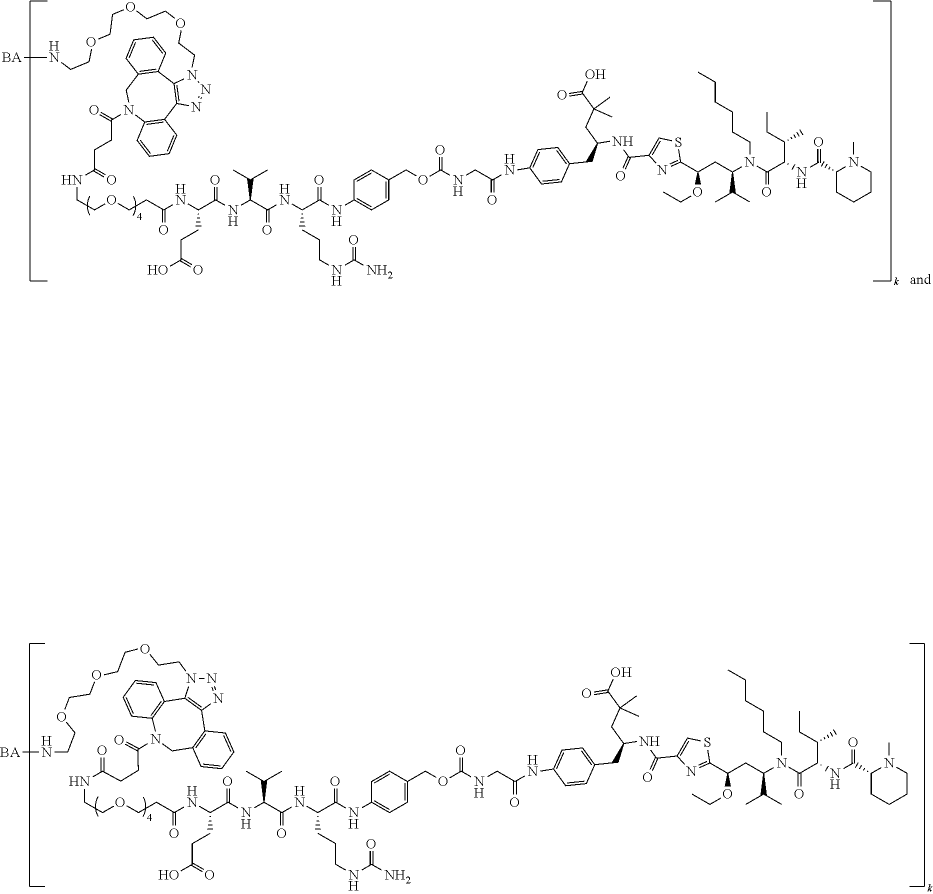

or a pharmaceutically acceptable salt thereof, wherein BA is a binding agent; and k is one, two, three, or four.

In certain embodiments, an antibody or antigen-binding fragment thereof can be conjugated directly, or via a linker, to any one or more of Formulae I, Ia, II, III, IV, V, and/or VI as described herein. In one embodiment, an antibody-drug conjugate includes an antibody or antigen binding fragment thereof conjugated to any one or more of Formulae I, Ia, II, III, IV, V, and/or VI as described herein, selected from the group consisting of

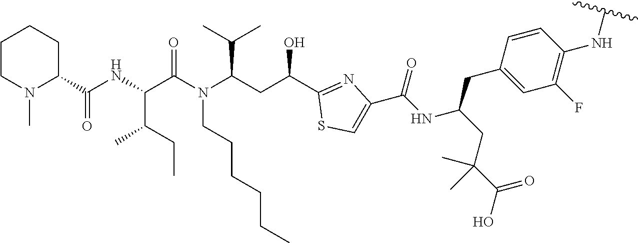 [SMILES: *Nc1ccc(C[C@@H](CC(C)(C)C(=O)O)NC(=O)c2csc([C@H](O)C[C@H](C(C)C)N(CCCCCC)C(=O)[C@@H](NC(=O)[C@H]3CCCCN3C)[C@@H](C)CC)n2)cc1F]

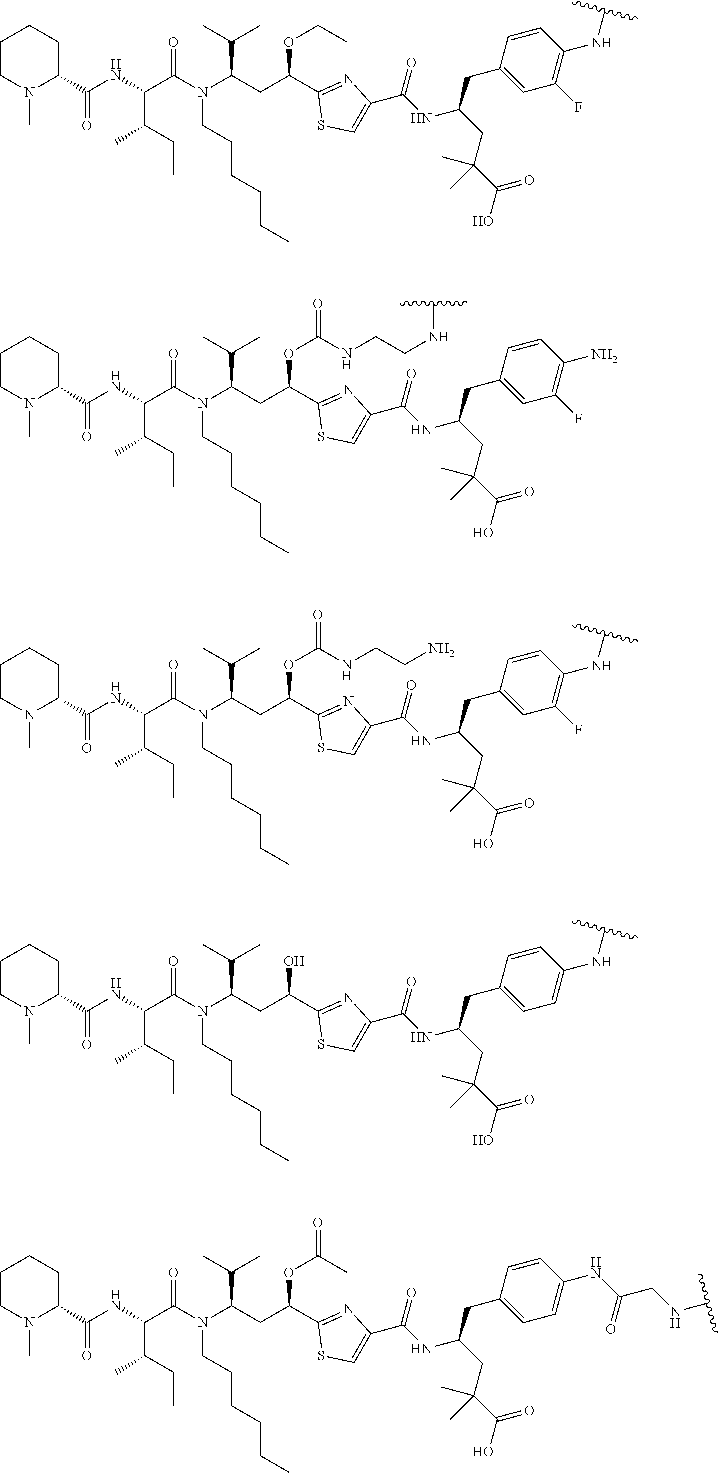

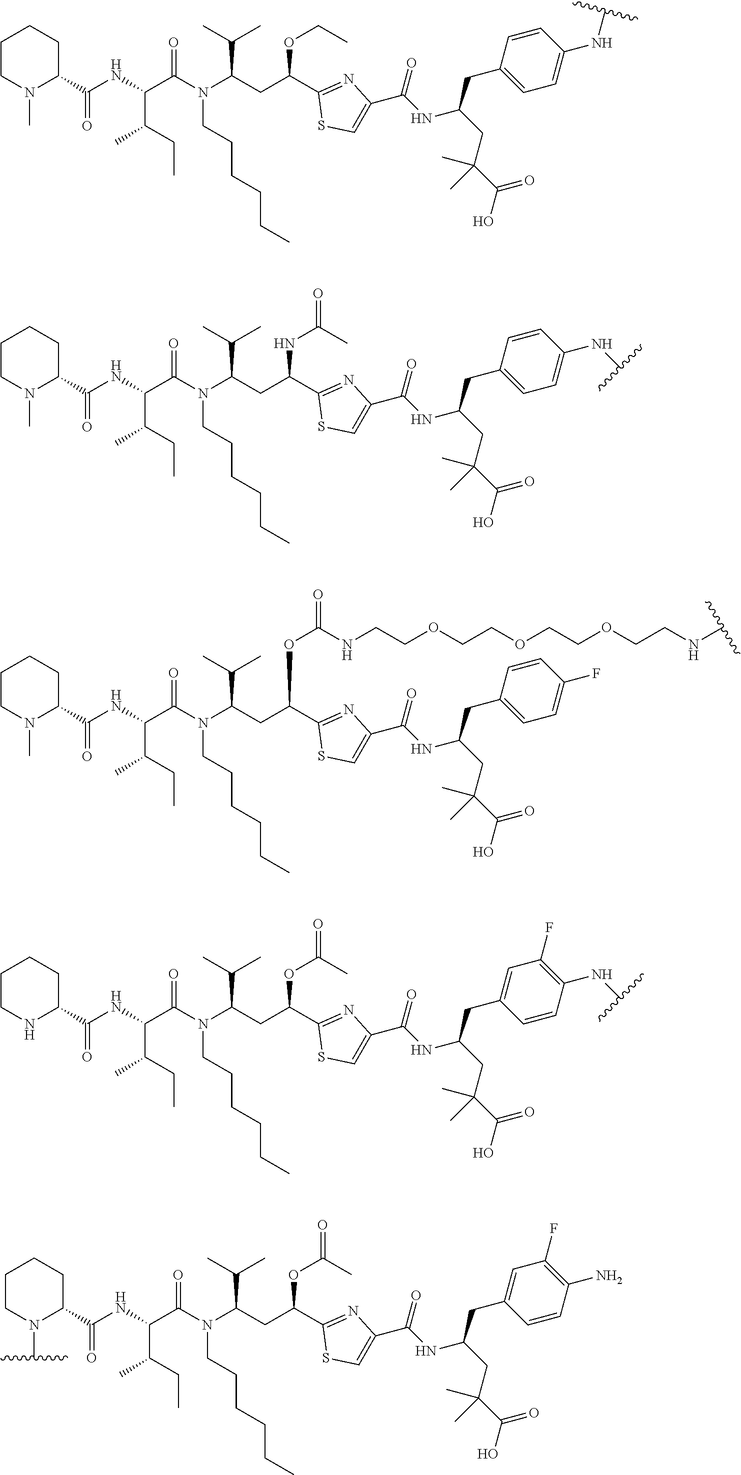

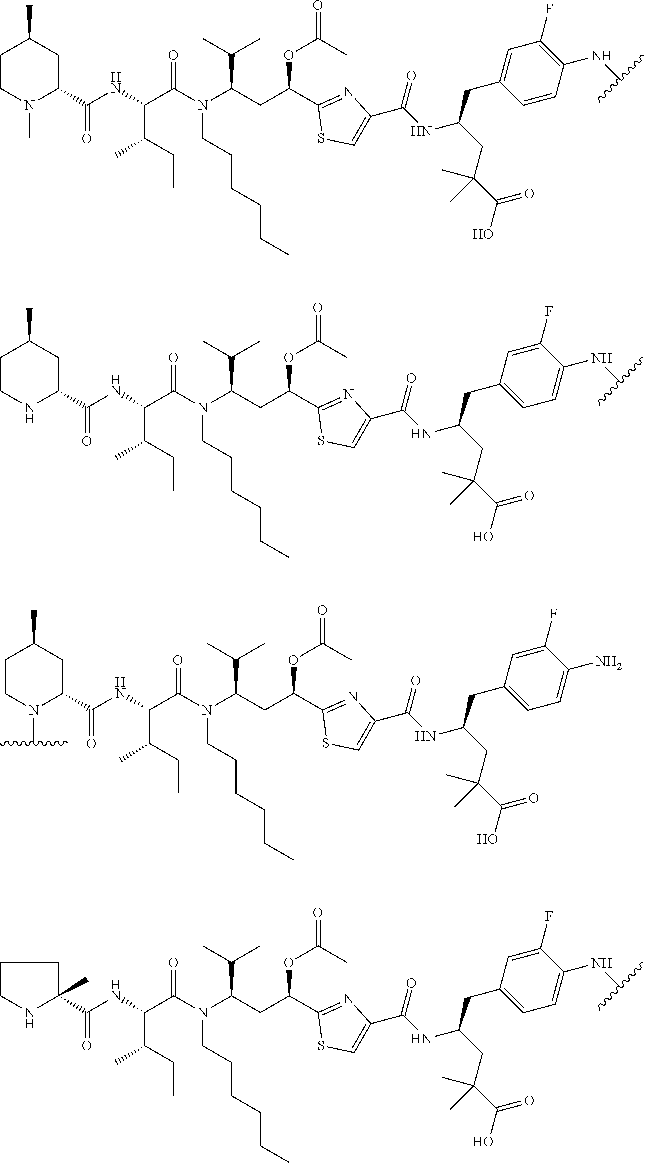

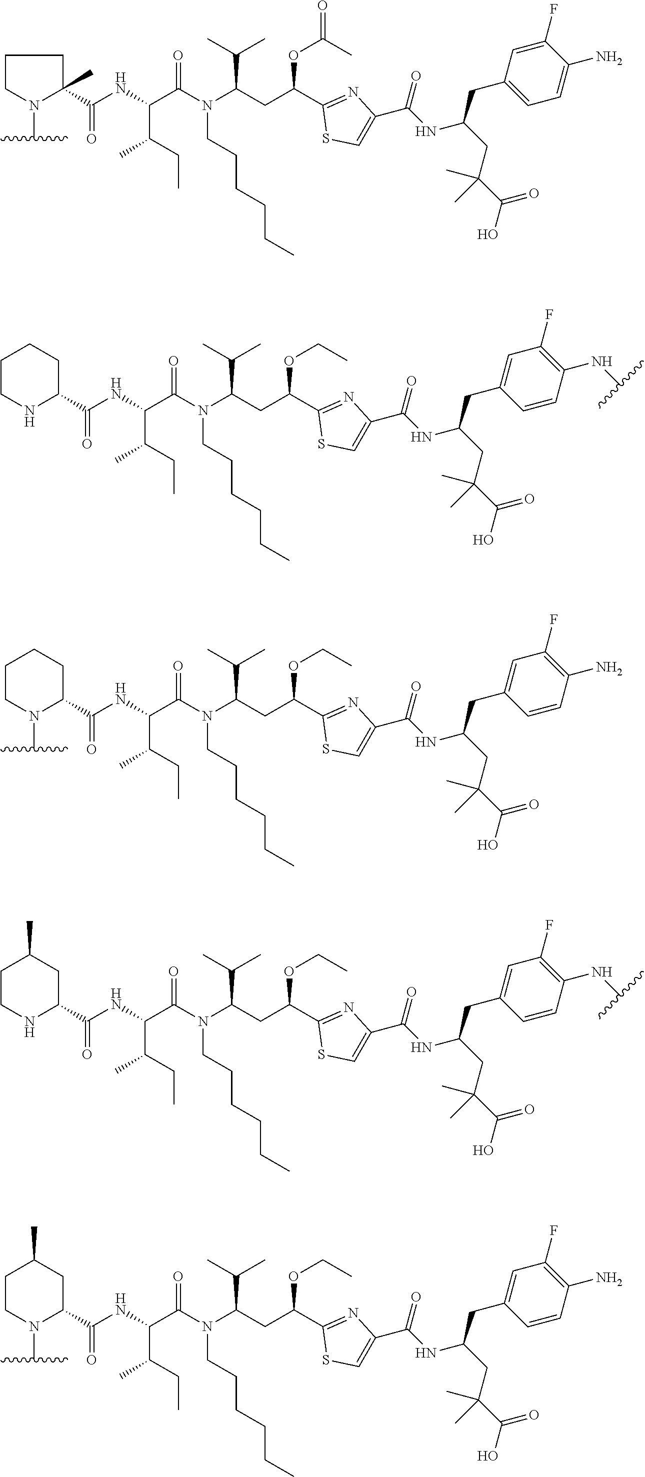

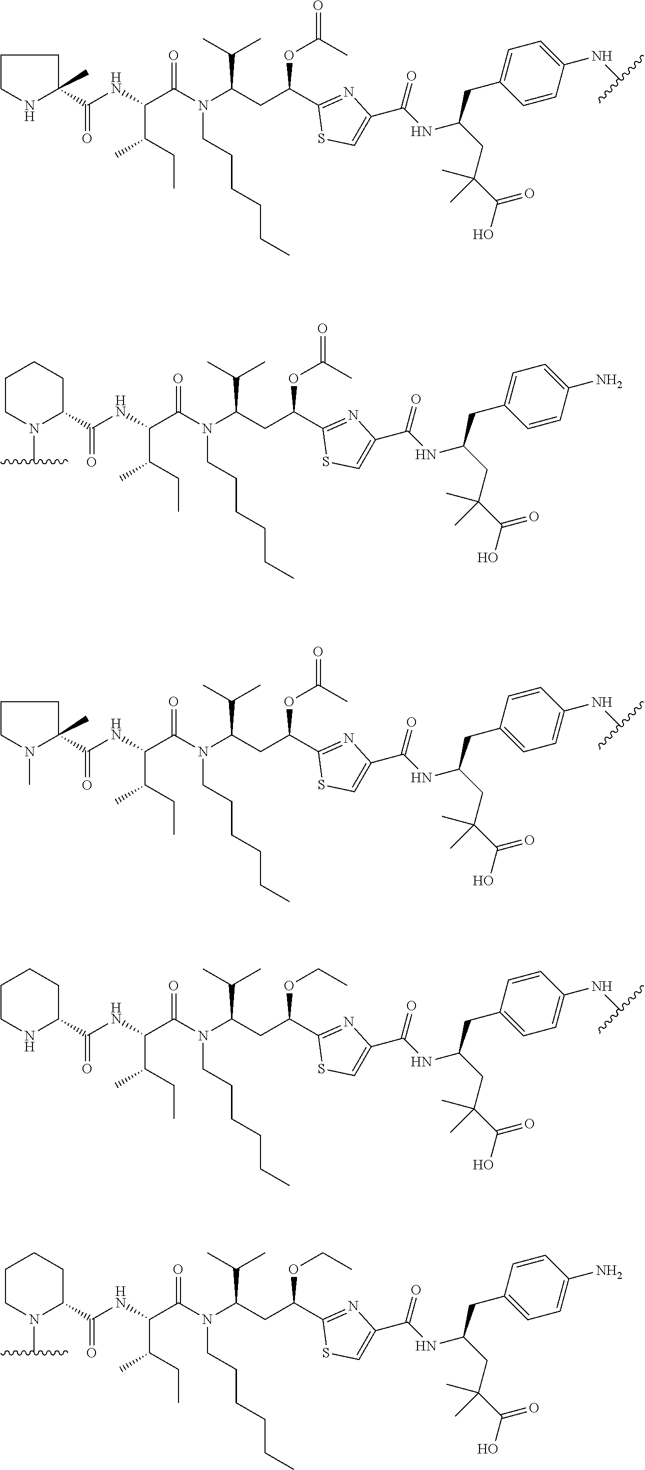

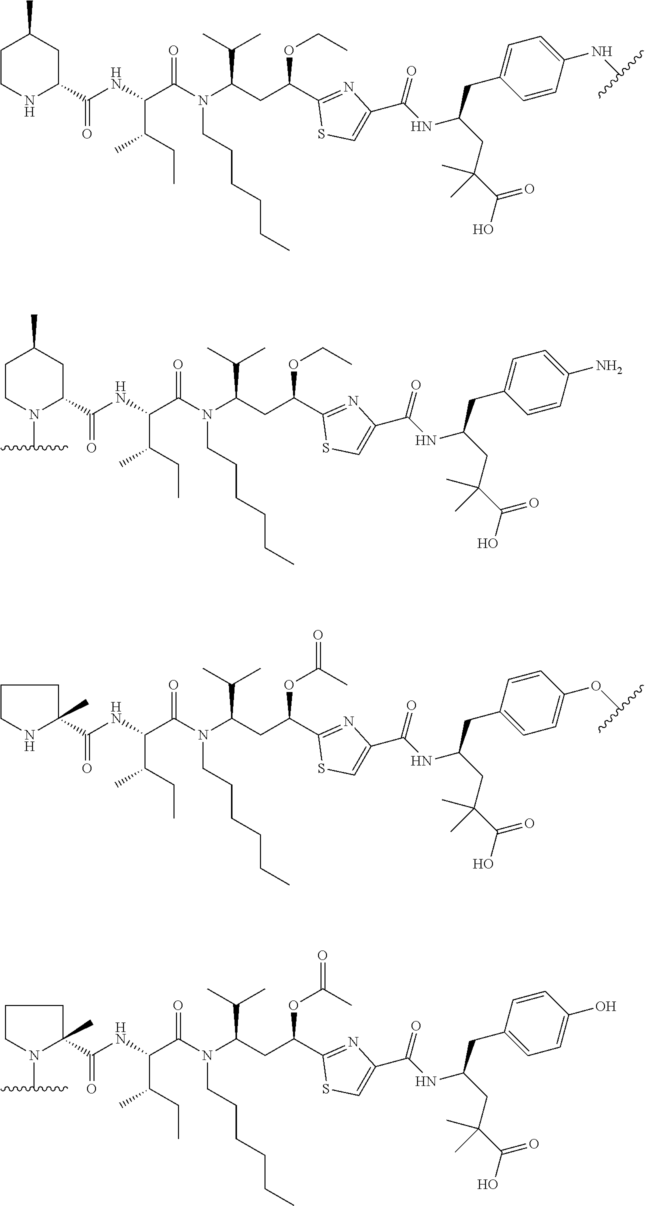

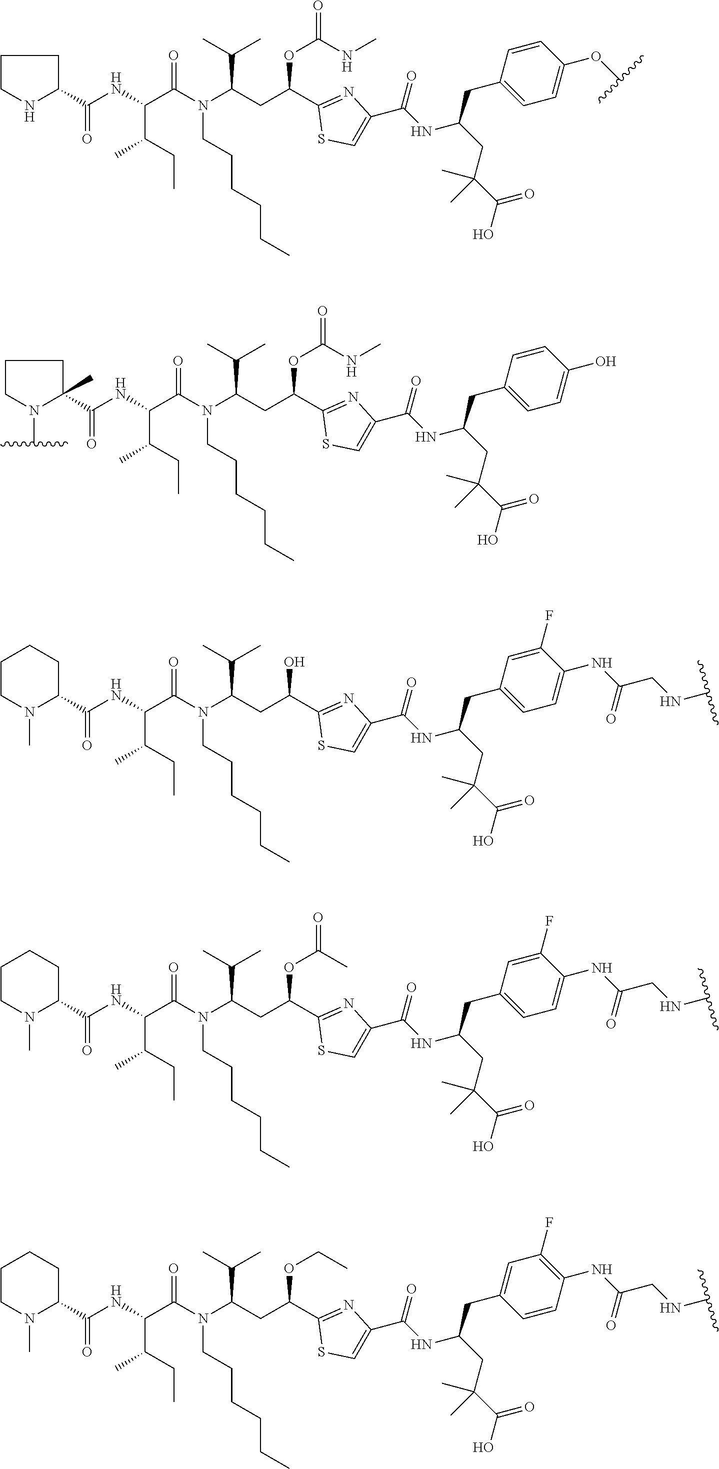

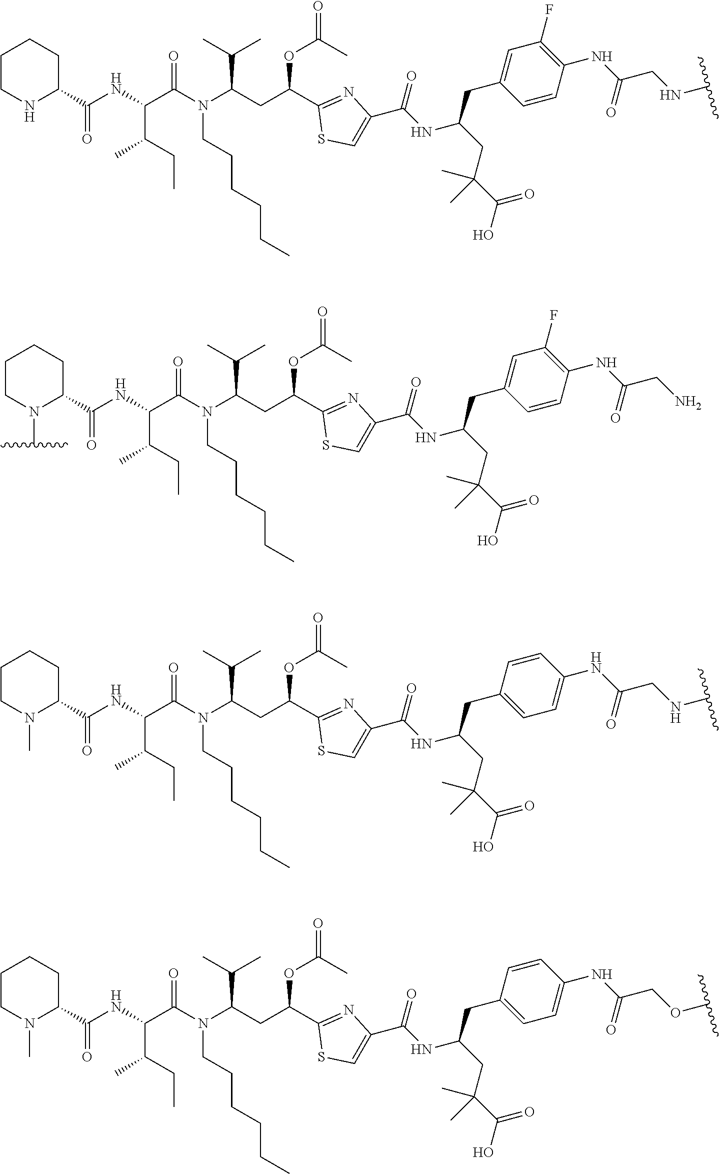

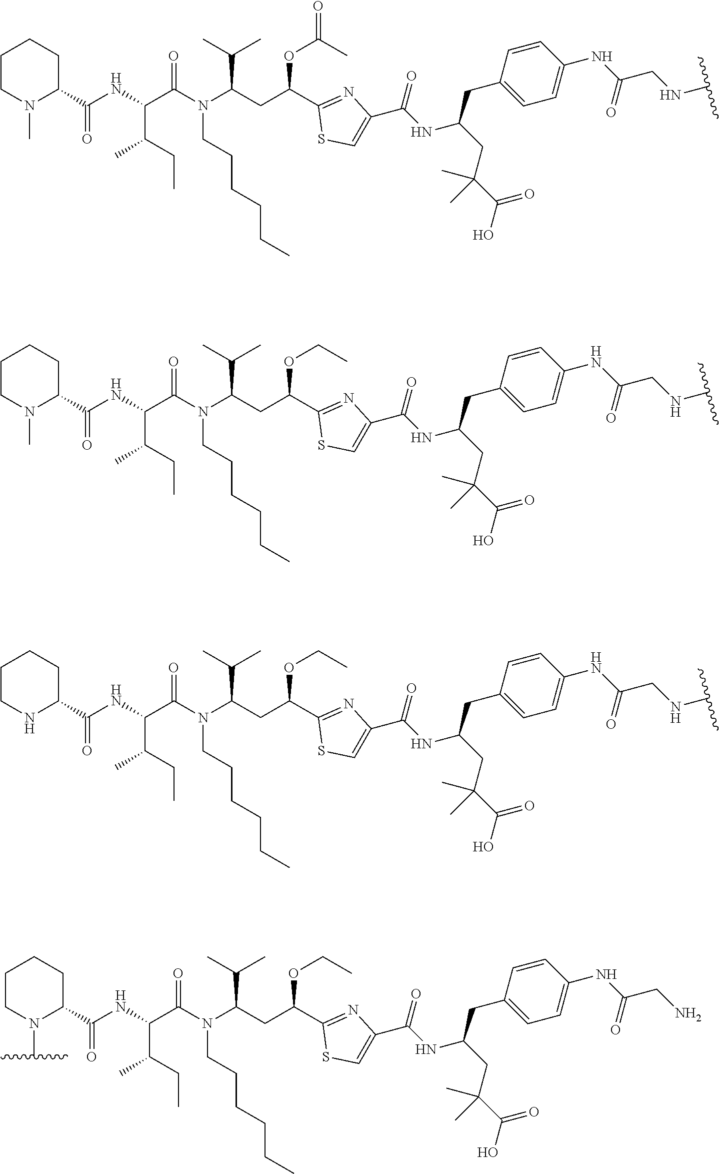

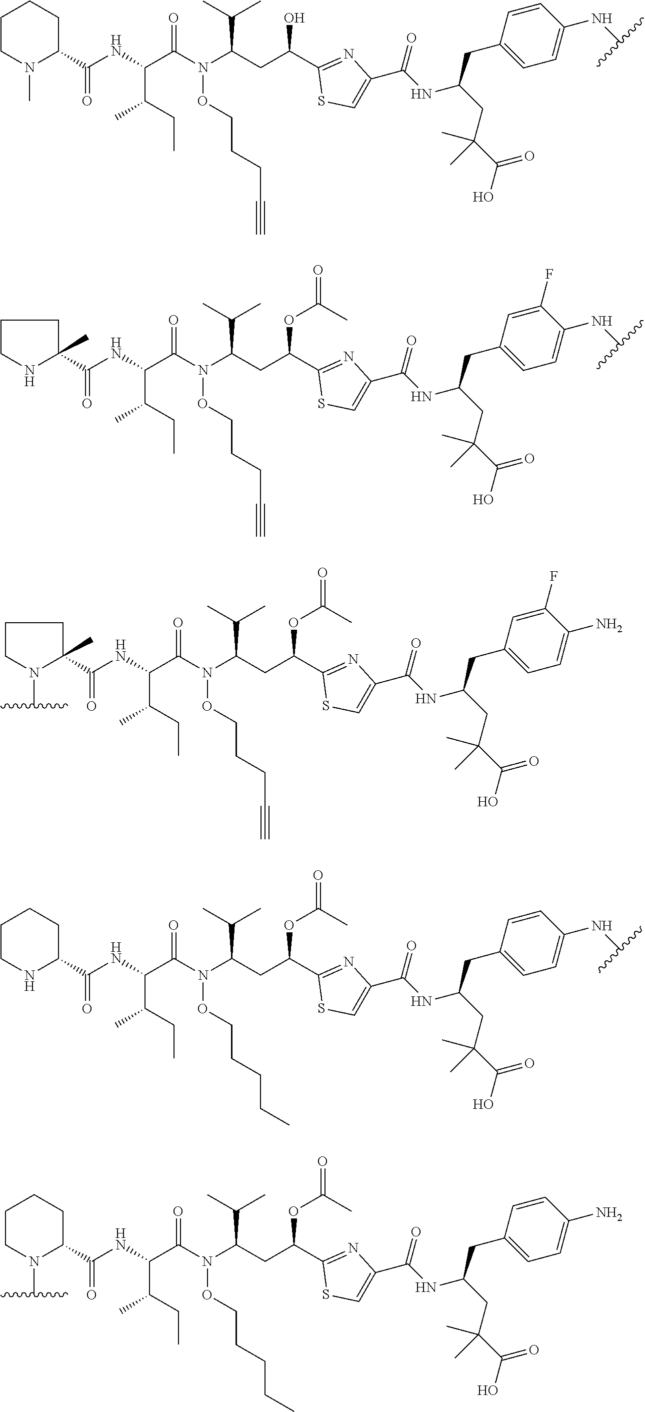

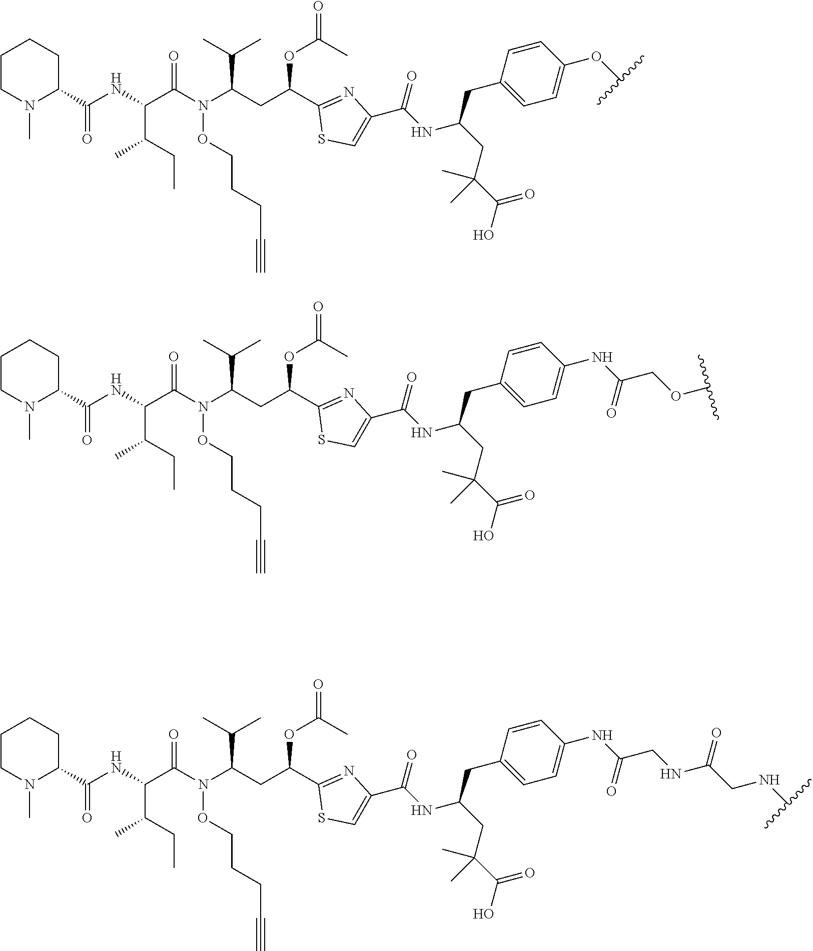 [SMILES: *Oc1ccc(C[C@@H](CC(C)(C)C(=O)O)NC(=O)c2csc([C@@H](C[C@H](C(C)C)N(OCCCC#C)C(=O)[C@@H](NC(=O)[C@H]3CCCCN3C)[C@@H](C)CC)OC(C)=O)n2)cc1 ; *OCC(=O)Nc1ccc(C[C@@H](CC(C)(C)C(=O)O)NC(=O)c2csc([C@@H](C[C@H](C(C)C)N(OCCCC#C)C(=O)[C@@H](NC(=O)[C@H]3CCCCN3C)[C@@H](C)CC)OC(C)=O)n2)cc1 ; *NCC(=O)NCC(=O)Nc1ccc(C[C@@H](CC(C)(C)C(=O)O)NC(=O)c2csc([C@@H](C[C@H](C(C)C)N(OCCCC#C)C(=O)[C@@H](NC(=O)[C@H]3CCCCN3C)[C@@H](C)CC)OC(C)=O)n2)cc1]

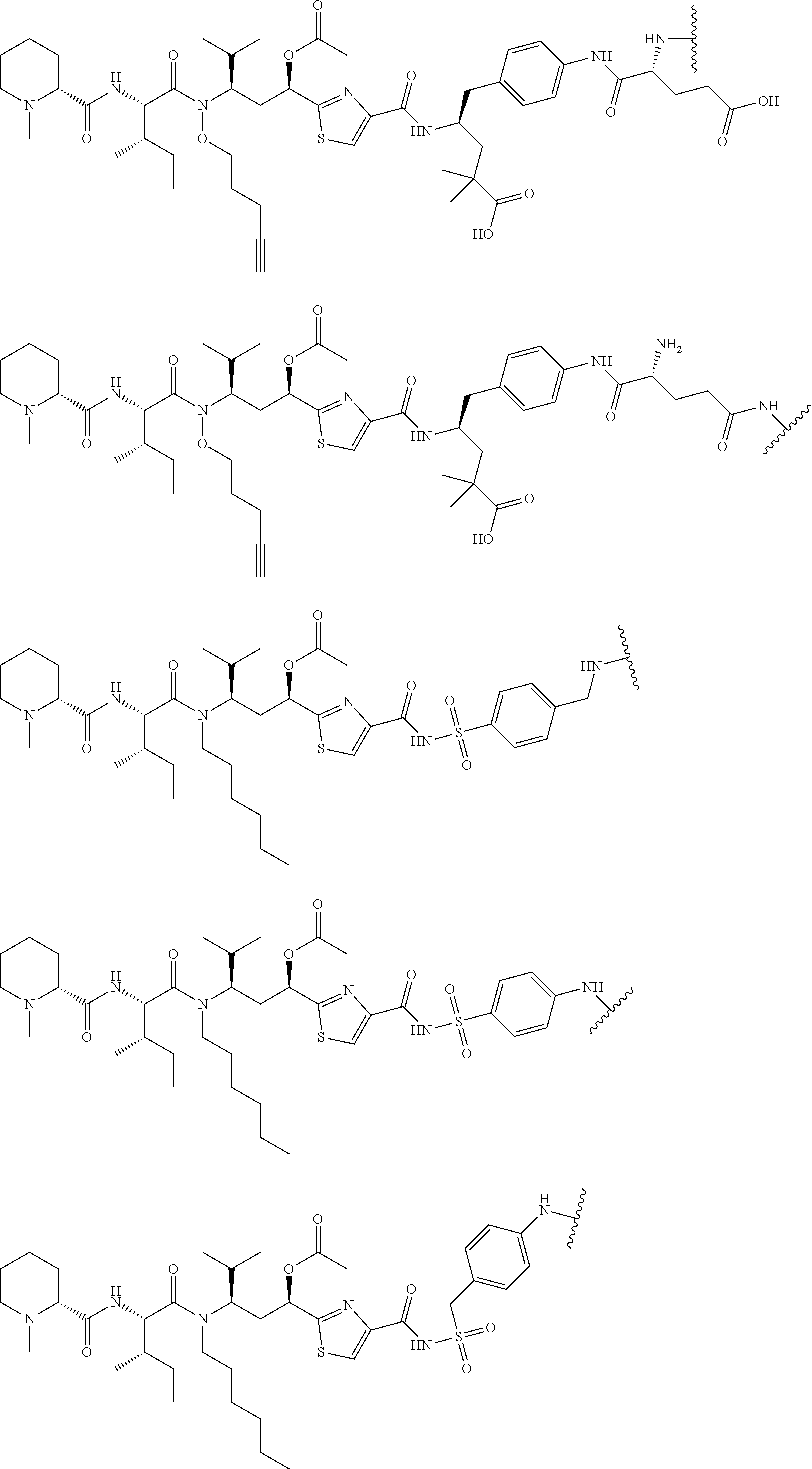

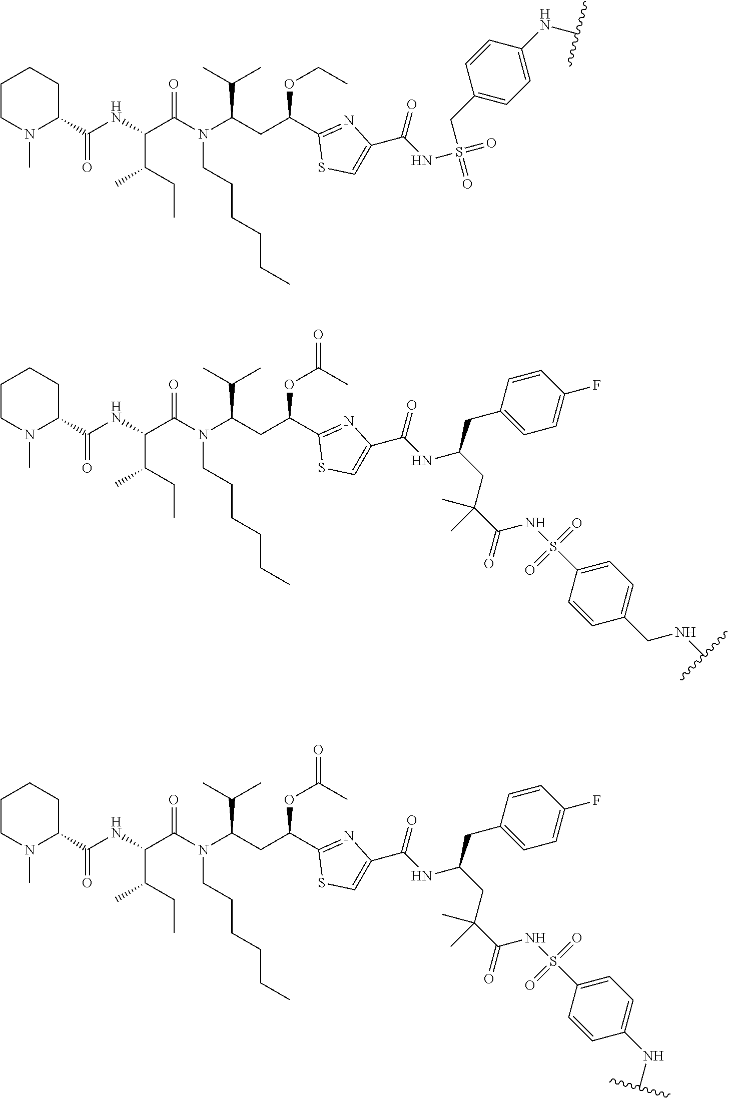 [SMILES: *Nc1ccc(CS(=O)(=O)NC(=O)c2csc([C@@H](C[C@H](C(C)C)N(CCCCCC)C(=O)[C@@H](NC(=O)[C@H]3CCCCN3C)[C@@H](C)CC)OCC)n2)cc1 ; *NCc1ccc(S(=O)(=O)NC(=O)C(C)(C)C[C@H](Cc2ccc(F)cc2)NC(=O)c2csc([C@@H](C[C@H](C(C)C)N(CCCCCC)C(=O)[C@@H](NC(=O)[C@H]3CCCCN3C)[C@@H](C)CC)OC(C)=O)n2)cc1 ; *Nc1ccc(S(=O)(=O)NC(=O)C(C)(C)C[C@H](Cc2ccc(F)cc2)NC(=O)c2csc([C@@H](C[C@H](C(C)C)N(CCCCCC)C(=O)[C@@H](NC(=O)[C@H]3CCCCN3C)[C@@H](C)CC)OC(C)=O)n2)cc1]

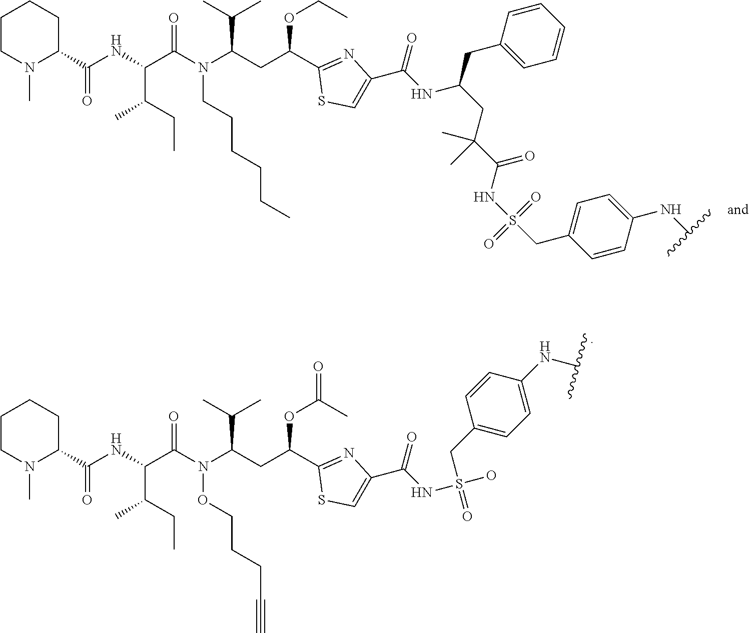

In any of the compound or conjugate embodiments provided, BA is an antibody, or antigen binding fragment thereof, that binds PRLR. In any of the compound or conjugate embodiments provided, BA is an antibody, or antigen binding fragment thereof, that binds STEAP2. In any of the compound or conjugate embodiments provided, BA is an antibody or antigen-binding fragment thereof, and conjugation is through at least one Q295 residue. In any of the compound or conjugate embodiments provided, BA is an antibody or antigen-binding fragment thereof, and conjugation is through two Q295 residues. In any of the compound or conjugate embodiments provided, BA is a N297Q antibody or antigen-binding fragment thereof. In any of the compound or conjugate embodiments provided, BA is a N297Q antibody or antigen-binding fragment thereof, and conjugation is through at least one Q295 and at least one Q297 residue. In any of the compound or conjugate embodiments provided, BA is a N297Q antibody or antigen-binding fragment thereof, and conjugation is through two Q295 residues and two Q297 residues. In particular embodiments, numbering is according to the EU numbering system.

In any of the embodiments above, BA is an anti-STEAP2 antibody. In certain embodiments, BA is the anti-STEAP2 antibody H1H7814N described in the Examples below. In certain embodiments, BA is the anti-STEAP2 antibody H1H7814N N297Q described in the Examples below. In certain embodiments, BA is an anti-STEAP2 antibody comprising an HCVR according to SEQ ID NO:1 and an LCVR according to SEQ ID NO:5. In certain embodiments, BA is an N297Q antibody comprising an HCVR according to SEQ ID NO:1 and an LCVR according to SEQ ID NO:5. In certain embodiments, BA is an anti-STEAP2 antibody comprising one, two, three, four, five, or six of HCDR1, HCDR2, HCDR3, LCDR1, LCDR2, and LCDR3 according to SEQ ID NOS:2, 3, 4, 6, 7, and 8, respectively. In certain embodiments, BA is an N297Q antibody comprising one, two, three, four, five, or six of HCDR1, HCDR2, HCDR3, LCDR1, LCDR2, and LCDR3 according to SEQ ID NOS:2, 3, 4, 6, 7, and 8, respectively. N297Q indicates that one or more residues 297 are mutated from asparagine (N) to glutamine (Q). In certain embodiments, each residue 297 is mutated to Q. In certain embodiments, numbering is according to the EU numbering system. In certain embodiments of this paragraph, k is from 1 to 4. In certain embodiments, k is 1, 2, 3, or 4. In certain embodiments, k is 4.

In any of the embodiments above, BA is an anti-PRLR antibody. In certain embodiments, BA is the anti-PRLR antibody H1H6958N2 described in the Examples below. In certain embodiments, BA is the anti-PRLR antibody H1H6958N2 N297Q described in the Examples below. In certain embodiments, BA is an anti-PRLR antibody comprising an HCVR according to SEQ ID NO:9 and an LCVR according to SEQ ID NO:13. In certain embodiments, BA is an N297Q antibody comprising an HCVR according to SEQ ID NO:9 and an LCVR according to SEQ ID NO:13. In certain embodiments, BA is an anti-PRLR antibody comprising one, two, three, four, five, or six of HCDR1, HCDR2, HCDR3, LCDR1, LCDR2, and LCDR3 according to SEQ ID NOS:10, 11, 12, 14, 15, and 16, respectively. In certain embodiments, BA is an N297Q antibody comprising one, two, three, four, five, or six of HCDR1, HCDR2, HCDR3, LCDR1, LCDR2, and LCDR3 according to SEQ ID NOS:10, 11, 12, 14, 15, and 16, respectively. N297Q indicates that one or more residues 297 are mutated from asparagine (N) to glutamine (Q). In certain embodiments, each residue 297 is mutated to Q. In certain embodiments, numbering is according to the EU numbering system. In certain embodiments of this paragraph, k is from 1 to 4. In certain embodiments, k is 1, 2, 3, or 4. In certain embodiments, k is 4.

In any preceding embodiment in this section, R⁷ is —NR^{7a}R^{7b}, wherein R^{7a} and R^{7b} are independently in each instance, hydrogen, alkyl, alkenyl, alkynyl, cycloalkyl, aryl, heteroaryl, acyl, and amino acid residue, wherein alkyl, alkenyl, alkynyl, cycloalkyl, aryl, heteroaryl, and acyl are optionally substituted. In certain embodiments R^{7a} is hydrogen and R^{7b} is an amino acid residue.

### Methods of Preparing Compounds or Payloads, and Linker-Payloads

The compounds provided herein can be prepared, isolated, or obtained by any method apparent to those of skill in the art. Exemplary methods of preparation are described in detail in the Examples below.

In certain embodiments, provided herein are compounds (e.g., linker-payloads or linker-prodrug payloads) selected from the group consisting of

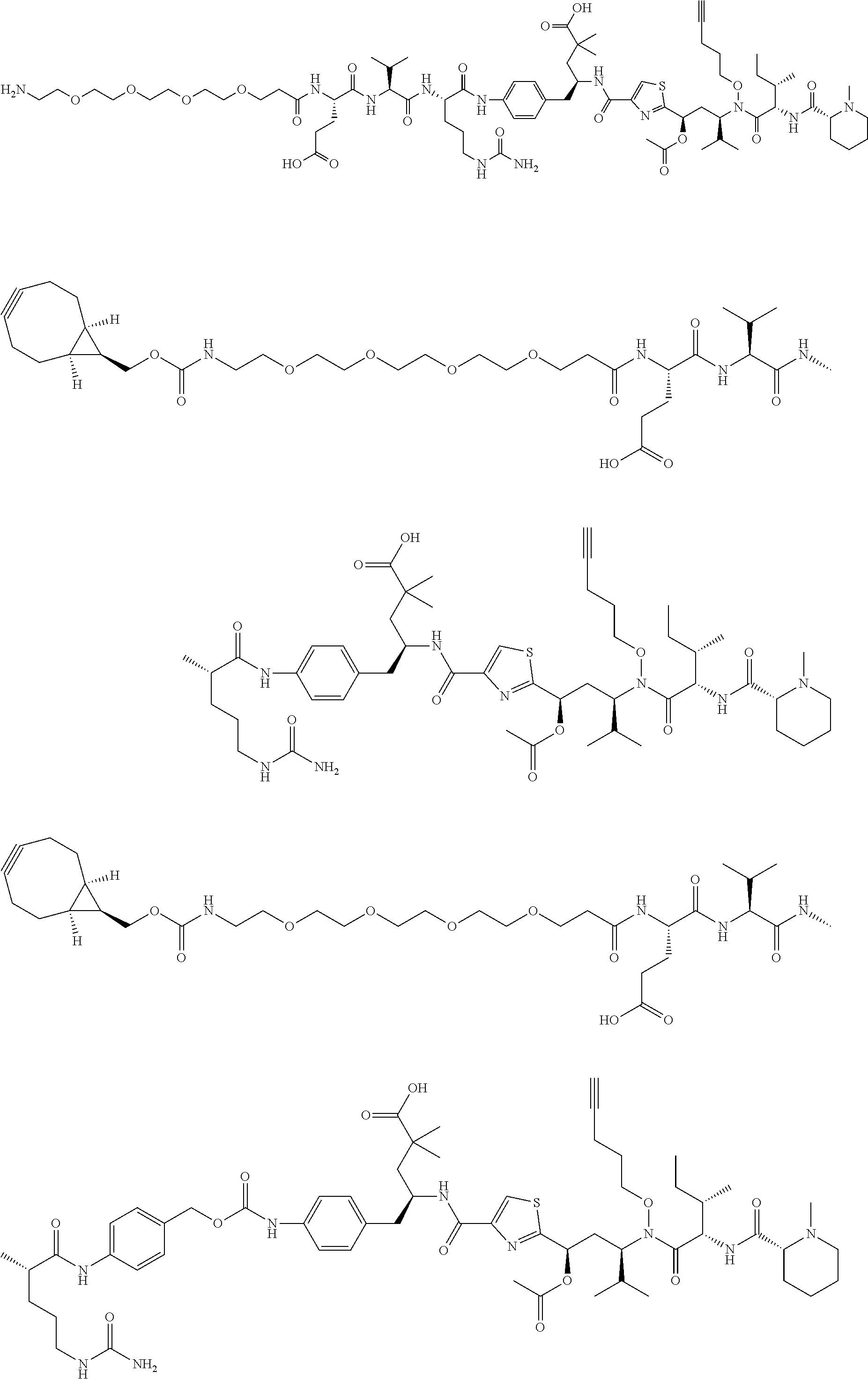

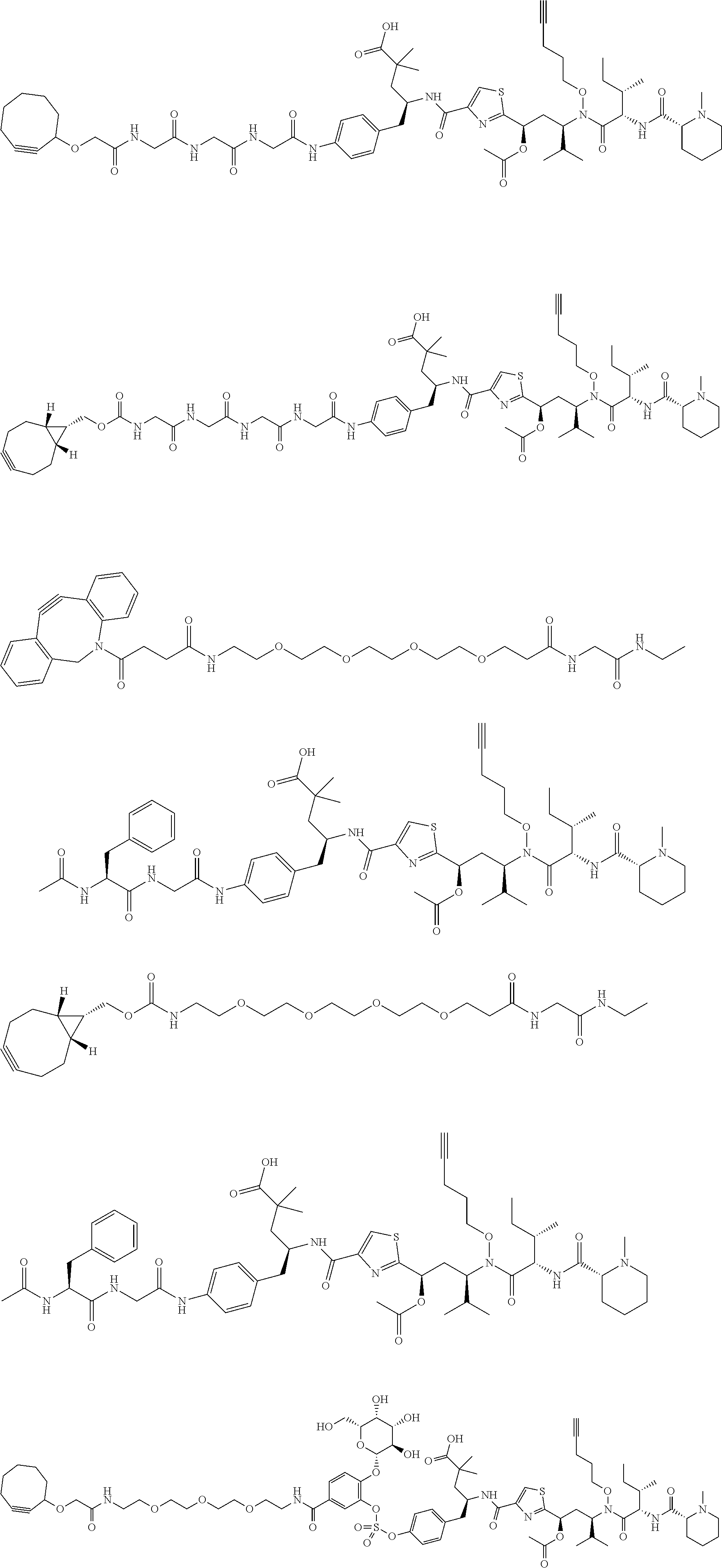

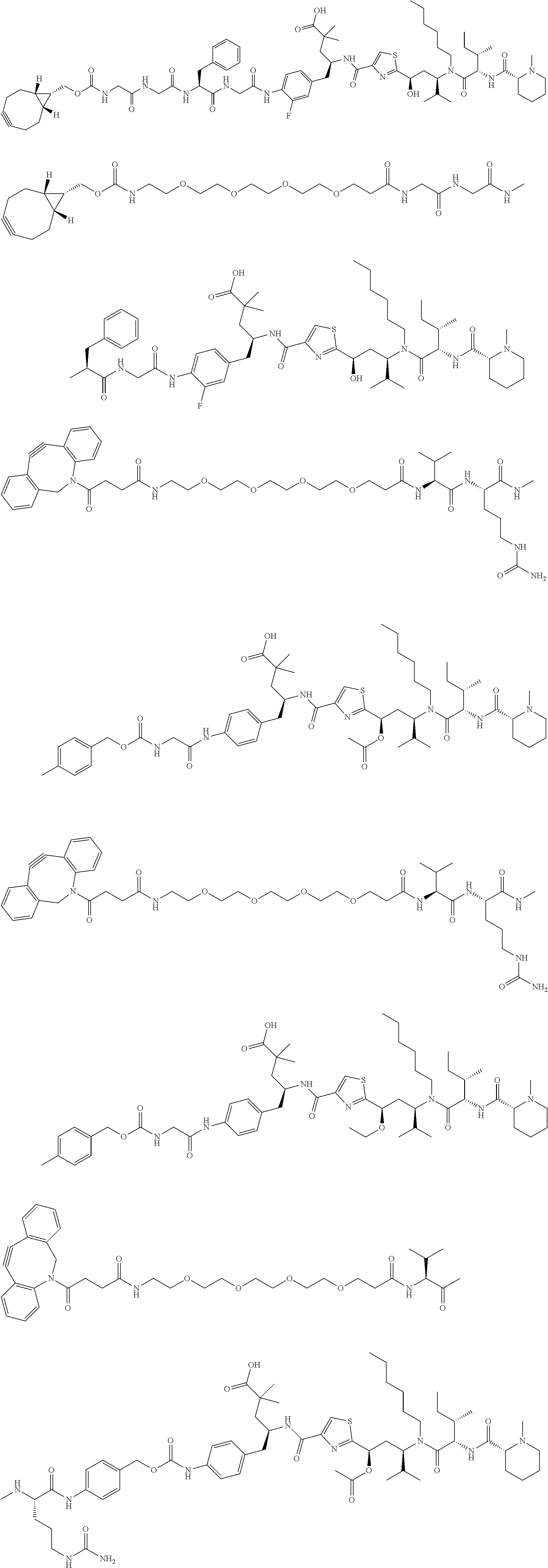

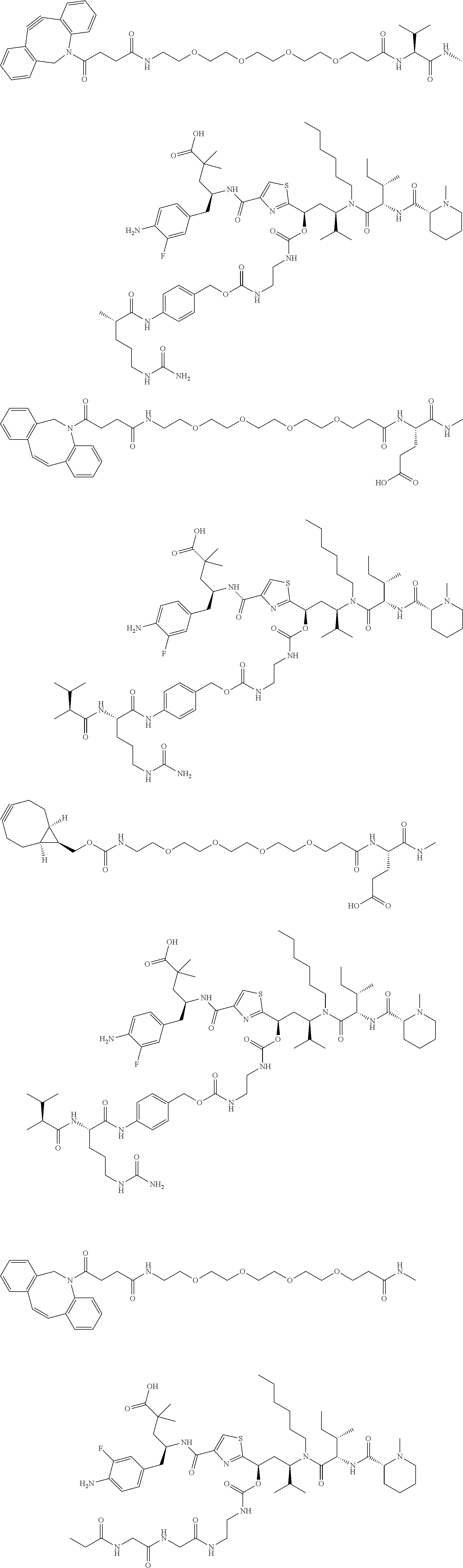

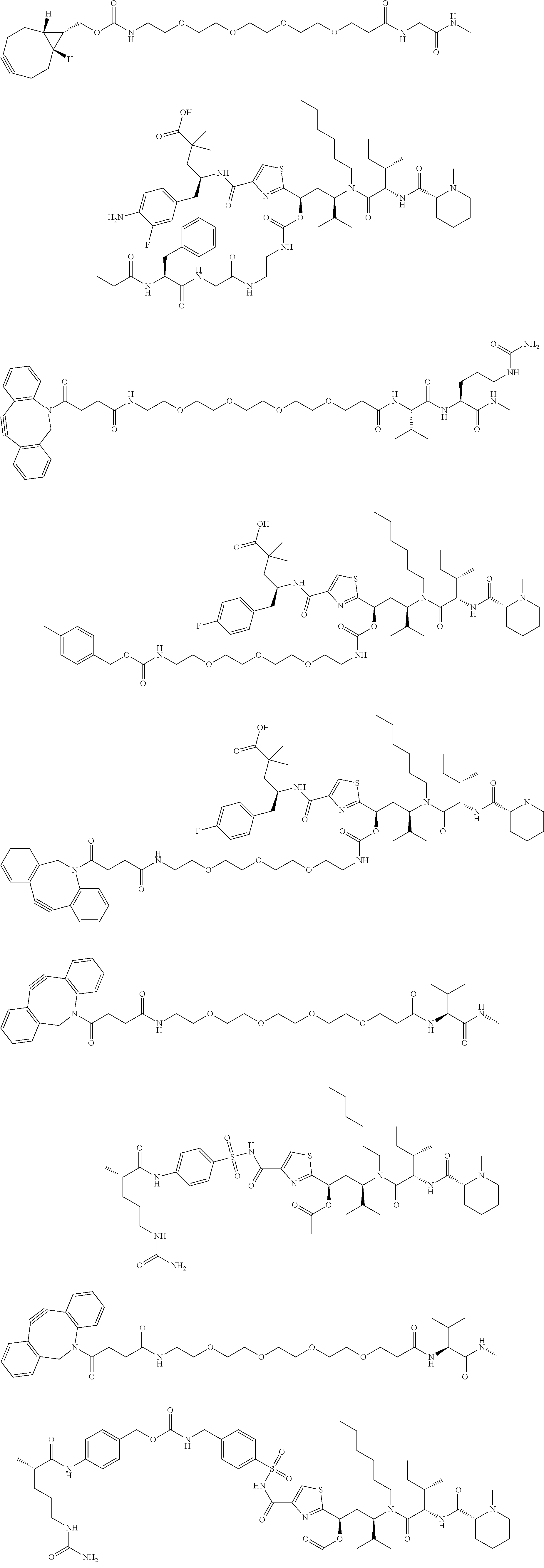

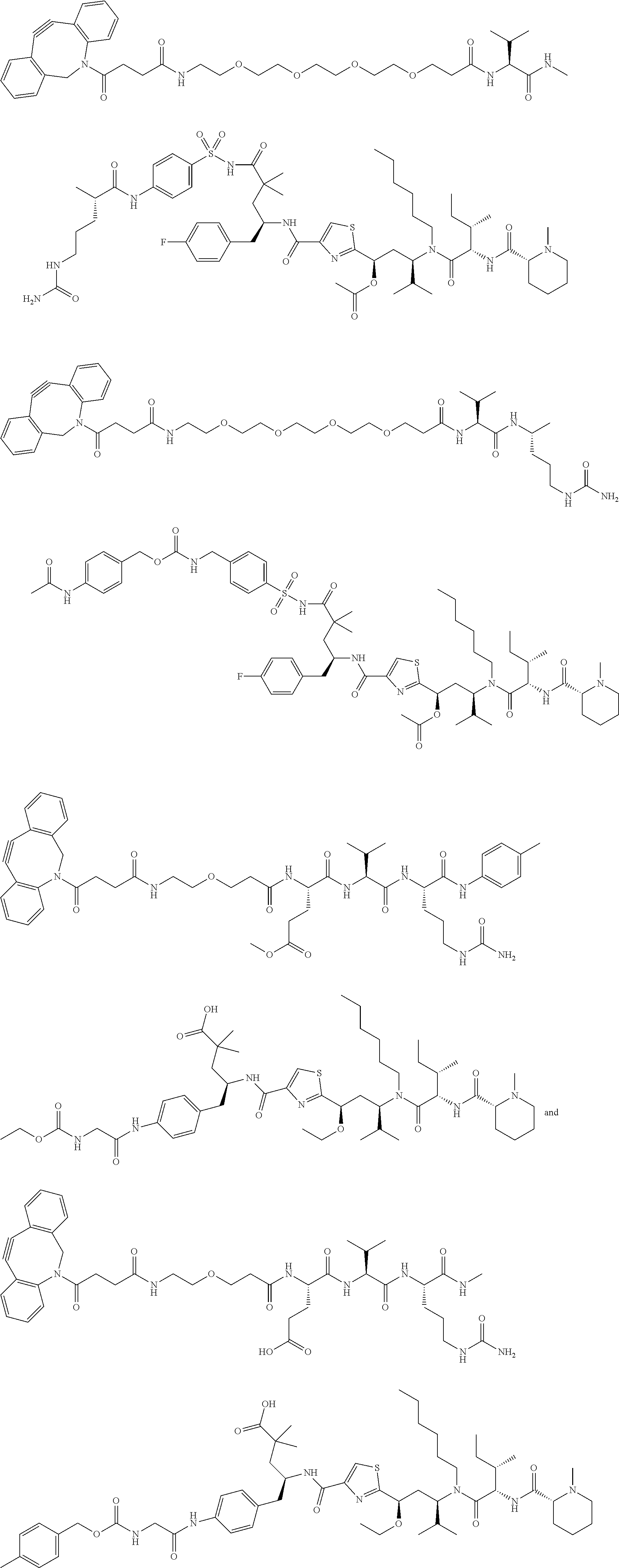

or a pharmaceutically acceptable salt thereof. In certain embodiments within this paragraph, all diastereomers are contemplated. For example, in one embodiment, the stereochemistry within

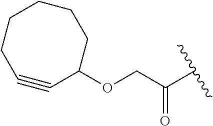 [SMILES: *C(=O)COC1C#CCCCCC1]

is undefined or racemic. By way of further example, in one embodiment, the stereochemistry within

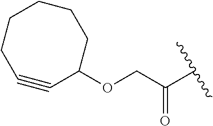 [SMILES: *C(=O)COC1C#CCCCCC1]

is (R)-. By way of further example, in one embodiment, the stereochemistry within

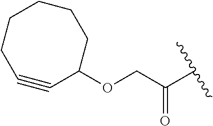 [SMILES: *C(=O)COC1C#CCCCCC1]

(S)-. By way of further example, in one embodiment, the stereochemistry within

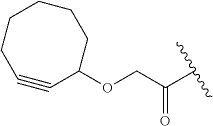 [SMILES: *C(=O)COC1C#CCCCCC1]

is (R)- in excess of (S)-. By way of further example, in one embodiment, the stereochemistry within

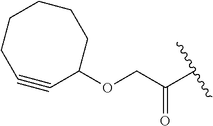 [SMILES: *C(=O)COC1C#CCCCCC1]

is (S)- in excess of (R)-.

The conjugates described herein can be synthesized by coupling the linker-payloads or linker-prodrug payloads described herein with a binding agent, for example, an antibody under standard conjugation conditions (see, e.g., Doronina et al. Nature Biotechnology 2003, 21, 778, which is incorporated herein by reference in its entirety). When the binding agent is an antibody, the antibody may be coupled to a linker-payload via one or more cysteine or lysine residues of the antibody. Linker-payloads can be coupled to cysteine residues, for example, by subjecting the antibody to a reducing agent, for example, dithiotheritol, to cleave the disulfide bonds of the antibody, purifying the reduced antibody, for example, by gel filtration, and subsequently treating the antibody with a linker-payload containing a suitable reactive moiety, for example, a maleimido group. Suitable solvents include, but are not limited to water, DMA, DMF, and DMSO. Linker-payloads or linker-prodrug payloads containing a reactive group, for example, an activated ester or acid halide group, can be coupled to lysine residues of the antibody. Suitable solvents include, but are not limited to, water, DMA, DMF, and DMSO. Conjugates can be purified using known protein techniques, including, for example, size exclusion chromatography, dialysis, and ultrafiltration/diafiltration.

Binding agents, for example antibodies, can also be conjugated via click chemistry reactions. In some embodiments of said click chemistry reactions, the linker-payload includes a reactive group, for example an alkyne, that is capable of undergoing a regioisomeric 1,3-cycloaddition reaction with an azide. Such suitable reactive groups are described above. The antibody includes one or more azide groups. Such antibodies include antibodies functionalized with, for example, azido-polyethylene glycol groups. In certain embodiments, such functionalized antibody is derived by treating an antibody having at least one glutamine residue, for example, heavy chain Gln295, with a primary amine compound in the presence of the enzyme transglutaminase (e.g., to generate a transglutaminase-modified antibody or antigen-binding fragment thereof). In certain embodiments, such functionalized or transglutaminase-modified antibody is derived by treating an antibody having at least one glutamine residue, for example, heavy chain Gln297, with a primary amine compound in the presence of the enzyme transglutaminase. Such antibodies include Asn297Gln (N297Q) mutants. In certain embodiments, such functionalized antibody is derived by treating an antibody having at least two glutamine residues, for example, heavy chain Gln295 and heavy chain Gln297, with a primary amine compound in the presence of the enzyme transglutaminase. Such antibodies include Asn297Gln (N297Q) mutants. In certain embodiments, the antibody has two heavy chains as described in this paragraph for a total of two or a total of four glutamine residues.

In certain embodiments, the antibody comprises two glutamine residues, one in each heavy chain. In particular embodiments, the antibody comprises a Q295 residue in each heavy chain. In further embodiments, the antibody comprises one, two, three, four, five, six, seven, eight, or more glutamine residues. These glutamine residues can be in heavy chains, light chains, or in both heavy chains and light chains. These glutamine residues can be wild-type residues, or engineered residues. The antibodies can be prepared according to standard techniques.

Those of skill will recognize that antibodies are often glycosylated at residue N297, near residue Q295 in a heavy chain sequence. Glycosylation at residue N297 can interfere with a transglutaminase at residue Q295 (Dennler et al., supra). Accordingly, in advantageous embodiments, the antibody is not glycosylated. In certain embodiments, the antibody is deglycoslated or aglycosylated. In particular embodiments, an antibody heavy chain has an N297 mutation. Alternatively stated, the antibody is mutated to no longer have an asparagine residue at position 297. In particular embodiments, an antibody heavy chain has an N297Q mutation. Such an antibody can be prepared by site-directed mutagenesis to remove or disable a glycosylation sequence or by site-directed mutagenesis to insert a glutamine residue at a site apart from any interfering glycosylation site or any other interfering structure. Such an antibody also can be isolated from natural or artificial sources.

The antibody without interfering glycosylation is then reacted or treated with a primary amine compound. In certain embodiments, an aglycosylated antibody is reacted or treated with a primary amine compound to produce a glutaminyl-modified antibody or transglutaminase-modified antibody. In certain embodiments, a deglycosylated antibody is reacted or treated with a primary amine compound to produce a glutaminyl-modified antibody or transglutaminase-modified antibody.

The primary amine can be any primary amine that is capable of forming a covalent bond with a glutamine residue in the presence of a transglutaminase. Useful primary amines are described herein. The transglutaminase can be any transglutaminase deemed suitable by those of skill in the art. In certain embodiments, the transglutaminase is an enzyme that catalyzes the formation of an isopeptide bond between a free amine group on the primary amine compound and the acyl group on the side chain of a glutamine residue. Transglutaminase is also known as protein-glutamine-7-glutamyltransferase. In particular embodiments, the transglutaminase is classified as EC 2.3.2.13. The transglutaminase can be from any source deemed suitable. In certain embodiments, the transglutaminase is microbial. Useful transglutaminases have been isolated from Streptomyces mobaraense, Streptomyces cinnamoneum, Streptomyces griseo-carneum, Streptomyces lavendulae, and Bacillus subtilis. Non-microbial transglutaminases, including mammalian transglutaminases, can also be used. In certain embodiments, the transglutaminase can be produced by any technique or obtained from any source deemed suitable by the practitioner of skill. In particular embodiments, the transglutaminase is obtained from a commercial source.

In particular embodiments, the primary amine compound comprises a reactive group capable of further reaction after transglutamination. In these embodiments, the glutaminyl-modified antibody or transglutaminase-modified antibody can be reacted or treated with a reactive payload or prodrug payload compound or a reactive linker-payload or linker-prodrug compound to form an antibody-payload conjugate or an antibody-linker-payload conjugate. In certain embodiments, the primary amine compound comprises an azide.

In certain embodiments, the glutaminyl-modified antibody or transglutaminase-modified antibody is reacted or treated with a reactive linker-payload to form an antibody-linker-payload conjugate. The reaction can proceed under conditions deemed suitable by those of skill in the art. In certain embodiments, the glutaminyl-modified antibody or transglutaminase-modified antibody is contacted with the reactive linker-payload or linker-prodrug payload compound under conditions suitable for forming a bond between the glutaminyl-modified antibody or transglutaminase-modified antibody and the linker-payload or linker-prodrug payload compound. Suitable reaction conditions are well known to those in the art. Exemplary reactions are provided in the Examples below.

### Pharmaceutical Compositions and Methods of Treatment

Provided herein are methods of treating and preventing diseases, conditions, or disorders comprising administering a therapeutically or prophylactically effective amount or one or more of the compounds disclosed herein, for example, one or more of the compounds of a formula provided herein. Diseases, disorders, and/or conditions include, but are not limited to, those associated with the antigens listed herein.

The compounds described herein can be administered alone or together with one or more additional therapeutic agents. The one or more additional therapeutic agents can be administered just prior to, concurrent with, or shortly after the administration of the compounds described herein. This disclosure also includes pharmaceutical compositions comprising any of the compounds described herein in combination with one or more additional therapeutic agents, and methods of treatment comprising administering such combinations to subjects in need thereof.

Suitable additional therapeutic agents include, but are not limited to, a second tubulysin, an autoimmune therapeutic agent, a hormone, a biologic, or a monoclonal antibody. Suitable therapeutic agents also include, but are not limited to any pharmaceutically acceptable salts, acids, or derivatives of a compound set forth herein.

In some embodiments of the methods described herein, multiple doses of a compound described herein (or a pharmaceutical composition comprising a combination of a compound described herein and any of the additional therapeutic agents mentioned herein) may be administered to a subject over a defined time course. The methods according to this embodiment of the disclosure comprise sequentially administering to a subject multiple doses of a compound described herein. As used herein, “sequentially administering” means that each dose of the compound is administered to the subject at a different point in time, e.g., on different days separated by a predetermined interval (e.g., hours, days, weeks, or months). This disclosure includes methods which comprise sequentially administering to the patient a single initial dose of a compound described herein, followed by one or more secondary doses of the compound, and optionally followed by one or more tertiary doses of the compound.

The terms “initial dose,” “secondary doses,” and “tertiary doses,” refer to the temporal sequence of administration of the compounds described herein. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses can all include the same amount the compound described herein, but generally can differ from one another in terms of frequency of administration. In certain embodiments, the amount of the compound included in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of this disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose the compound which is administered to a patient prior to the administration of the very next dose in the sequence with no intervening doses.

The methods according to this embodiment of the disclosure may comprise administering to a patient any number of secondary and/or tertiary doses of the compound. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient. The administration regimen may be carried out indefinitely over the lifetime of a particular subject, or until such treatment is no longer therapeutically needed or advantageous.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

This disclosure includes administration regimens in which 2 to 6 loading doses are administered to a patient at a first frequency (e.g., once a week, once every two weeks, once every three weeks, once a month, once every two months, etc.), followed by administration of two or more maintenance doses to the patient on a less frequent basis. For example, according to this embodiment of the disclosure, if the loading doses are administered at a frequency of once a month, then the maintenance doses may be administered to the patient once every six weeks, once every two months, once every three months, etc.

This disclosure includes pharmaceutical compositions of the compounds and/or conjugates described herein, e.g., the compounds Formulae I, Ia, II, III, IV, V, and VI, e.g., compositions comprising a compound described herein, a salt, stereoisomer, regioisomer, polymorph thereof, and a pharmaceutically acceptable carrier, diluent, and/or excipient. Examples of suitable carriers, diluents and excipients include, but are not limited to, buffers for maintenance of proper composition pH (e.g., citrate buffers, succinate buffers, acetate buffers, phosphate buffers, lactate buffers, oxalate buffers, and the like), carrier proteins (e.g., human serum albumin), saline, polyols (e.g., trehalose, sucrose, xylitol, sorbitol, and the like), surfactants (e.g., polysorbate 20, polysorbate 80, polyoxolate, and the like), antimicrobials, and antioxidants.

In some examples, set forth herein is a method of treating cancer comprising administering to a patient having said cancer a therapeutically effective amount of a compound of Formulae I, Ia, II, III, IV, V, and VI, or a pharmaceutical composition thereof. In some embodiments, provided herein is a method of treating cancer comprising administering to a patient having said cancer a therapeutically effective amount of a an antibody-tubulysin conjugate described herein, or a pharmaceutical composition thereof. In some embodiments, the binding agent, e.g., antibody, of the conjugates, e.g., antibody-drug conjugates described herein interact with or bind to tumor antigens, including antigens specific for a type of tumor or antigens that are shared, overexpressed, or modified on a particular type of tumor. Examples include, but are not limited to, alpha-actinin-4 with lung cancer, ARTC1 with melanoma, BCR-ABL fusion protein with chronic myeloid leukemia, B-RAF, CLPP or Cdc27 with melanoma, CASP-8 with squamous cell carcinoma, and hsp70-2 with renal cell carcinoma as well as the following shared tumor-specific antigens, for example, BAGE-1, GAGE, GnTV, KK-LC-1, MAGE-A2, NA88-A, TRP2-INT2. Further examples of tumor antigens include, but are not limited to, PSMA, PRLR, MUC16, HER2, EGFRvIII, and anti-STEAP2, and MET.

The compounds disclosed herein can be used for treating primary and/or metastatic tumors arising in the brain and meninges, oropharynx, lung and bronchial tree, gastrointestinal tract, male and female reproductive tract, muscle, bone, skin and appendages, connective tissue, spleen, immune system, blood forming cells and bone marrow, liver and urinary tract, and special sensory organs such as the eye. In certain embodiments, the compounds provided herein are used to treat one or more of the following cancers: renal cell carcinoma, pancreatic carcinoma, head and neck cancer (e.g., head and neck squamous cell carcinoma [HNSCC]), prostate cancer, castrate-resistant prostrate cancer, malignant gliomas, osteosarcoma, colorectal cancer, gastric cancer (e.g., gastric cancer with MET amplification), mesothelioma, malignant mesothelioma, multiple myeloma, ovarian cancer, lung cancer, small cell lung cancer, non-small cell lung cancer, synovial sarcoma, thyroid cancer, breast cancer, PRLR positive (PRLR+) breast cancer, melanoma, acute myelogenous leukemia, adult T-cell leukemia, astrocytomas, bladder cancer, cervical cancer, cholangiocarcinoma, endometrial cancer, esophageal cancer, glioblastomata, Kaposi's sarcoma, kidney cancer, leiomyosarcomas, liver cancer, lymphomas, MFH/fibrosarcoma, nasopharyngeal cancer, rhabdomyosarcoma, colon cancer, stomach cancer, uterine cancer, residual cancer wherein “residual cancer” means the existence or persistence of one or more cancerous cells in a subject following treatment with an anti-cancer therapy, and Wilms' tumor. In some embodiments, the cancer is breast cancer. In some embodiments, the cancer is prostate cancer.

In some examples, set forth herein is a method of preventing prostate cancer comprising administering to a patient having said disorder a prophylactically effective amount of a compound of Formulae I, Ia, II, III, IV, V, and VI, or a pharmaceutical composition thereof.

### EXAMPLES

Provided herein are novel tubulysins, protein conjugates thereof, and methods for treating diseases, disorders, and conditions including administering the tubulysins and conjugates.

Certain embodiments of this disclosure are illustrated by the following non-limiting examples. As used herein, the symbols and conventions used in these processes, schemes, and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the Examples, and throughout the specification:

| Abbreviation | Term or Phrase |
|---|---|
| ADC | Antibody-drug conjugate |
| Aglycosylated antibody | Antibody does not have any glycan |
| API | Atmospheric pressure ionization |
| aq | Aqueous |
| Boc | tert-butoxycarbonyl |
| COT | Cyclooctynol |
| CTRL | Antibody isotype control |
| Da | Dalton |
| DAD | Diode array detector |
| DAR | Drug to antibody ratio |
| DCM | Dichloromethane |
| DIBAC | 11,12-didehydro-5,6-dihydro-Dibenz[b,f]azocine |
| DIBAC-Suc | 11,12-didehydro-5,6-dihydro-Dibenz[b,f]azocine succinamic |
| | acid |
| DIBAC-Suc-PEG4- | {4-[(2S)-2-[(2S)-2-[1-(4-{2-azatricyclo[10.4.0.0^{4,9}]hexadeca- |
| VC-pAB-PNP | 1(12),4(9),5,7,13,15-hexaen-10-yn-2-yl}-4-oxobutanamido)- |
| | 3,6,9,12-tetraoxapentadecan-15-amido]-3-methylbutanamido]- |
| | 5-(carbamoylamino)pentanamido]phenyl}methyl 4-nitrophenyl |
| | carbonate |
| DIBACT | 3H-Benzo[c]-1,2,3-triazolo[4,5-e][1]benzazocine, 8,9-dihydro- |
| DIPEA | Diisopropylethylamine |
| DMF | N,N-dimethylformamide |
| DMSO | Dimethylsulfoxide |
| EC | Enzyme commission |
| ELSD | Evaporative light scattering detector |
| ESI | Electrospray ionization |
| Fmoc | N-(9-fluorenylmethyloxycarbonyl) |
| Fmoc-vcPAB-PNP | N-Fmoc-L-valine-L-citrulline-p-aminobenzyl alcohol p- |
| | nitrophenyl carbonate |
| g | Gram |
| HATU | 2-(7-Aza-1H-benzotriazole-1-yl)-1,1,3,3-tetramethyluronium |
| | hexafluorophosphate |
| HC | Heavy chain of immunoglobulin |
| HEK | Human embryonic kidney (cells) |
| HPLC | High performance liquid chromatography |
| hr, h, or hrs | Hours |
| LC | Light chain of immunoglobulin |
| MC | Maleimidocaproyl |
| mg | milligrams |
| min | minutes |
| mL | milliliters |
| mmh | myc-myc-hexahistidine tag |
| μL | microliters |
| mM | millimolar |
| μM | micromolar |
| MMAE | Monomethyl auristatin E |
| MS | Mass spectrometry |
| MsCl | Methanesulfonyl chloride |
| MSD | Mass-selective detector |
| MTG | Microbial transglutaminase (MTG EC 2.3.2.13, Zedira, Darmstadt, |
| | Germany) |
| MW | Molecular weight |
| ncADC | Non-Cytotoxic antibody drug conjugate |
| NHS | N-hydroxy succinimide |
| nM | nanomolar |
| NMR | Nuclear magnetic resonance |
| PAB | Para-aminobenzyloxy(carbonyl) |
| PBS | 10 mM sodium phosphate buffer and 150 mM sodium chloride |
| PBSg | 10 mM phosphate, 150 mM sodium chloride, 5% glycerol |
| PEG | Polyethyleneglycol |
| PNP | p-nitrophenyl |
| MC-VC-PAB-PNP | Maleimidocaproyl-L-valine-L-citrulline-p-aminobenzyl alcohol p- |
| | nitrophenyl carbonate |
| ppm | Parts per million (chemical shift, δ) |
| RP | Reversed phase |
| rt or RT | room temperature |
| SDS-PAGE | Sodium dodecylsulfate polyacrylamide gel electrophoresis |
| SEC | Size exclusion chromatography |
| Suc | Succinic acid |
| TCEP | Tris(2-carboxyethyl)phosphine hydrochloride |
| TEA | Triethylamine |
| TMS | tetramethylsilane |
| TFA | Trifluoroacetic acid |
| TG | Transglutaminase |
| THF | Tetrahydrofuran |
| TOF | Time-of-flight |
| TRSQ | Trastuzumab N297Q |
| UPLC | Ultra Performance Liquid Chromatography |
| UV | Ultraviolet |
| VA | Valine-alanine |
| VC | Valine-citrulline |
| VC-PAB | Valine-citrulline-para-aminobenzyloxy(carbonyl) |
| ZP3A | Azido-PEG₃-NH₂ or a residue thereof |

Reagents and solvents can be obtained from commercial sources such as Sinopharm Chemical Reagent Co. (SCRC), Sigma-Aldrich, Alfa, or other vendors, unless explicitly stated otherwise. ¹H NMR and other NMR spectra can be recorded on a Bruker AVIII 400 or Bruker AVIII 500. The data can be processed with Nuts software or MestReNova software, measuring proton shifts in parts per million (ppm) downfield from an internal standard tetramethylsilane (TMS).

HPLC-MS measurements can be run on an Agilent 1200 HPLC/6100 SQ System using the following conditions: Method A for HPLC-MS measurements include, as the Mobile Phase: A: Water (0.01% trifluoroacetic acid (TFA)), B: acetonitrile (0.01% TFA); Gradient Phase: 5% of B increases to 95% of B within 15 min; Flow Rate: 1.0 mL/min; Column: SunFire C18, 4.6×50 mm, 3.5 μm; Column Temperature: 50° C. Detectors: Analog to Digital Converter (ADC) Evaporative Light-scattering Detector (ELSD), Diode array detector (DAD) (214 nm and 254 nm), electrospray ionization-atmospheric ionization (ES-API). Method B for HPLC-MS measurements include, as the Mobile Phase: A: Water (10 mM NH₄HCO₃), B: acetonitrile; Gradient Phase: 5% to 95% of B within 15 min; Flow Rate: 1.0 mL/min; Column: XBridge C18, 4.6×50 mm, 3.5 μm; Column Temperature: 50° C. Detectors: ADC ELSD, DAD (214 nm and 254 nm), mass selective detector (MSD) (ES-API).

LC-MS measurements can be run on an Agilent 1200 HPLC/6100 SQ System using the following conditions: Method A for LC-MS measurements include, as the Instrument: WATERS 2767; column: Shimadzu Shim-Pack, PRC-ODS, 20×250 mm, 15 μm, two connected in series; Mobile Phase: A: Water (0.01% TFA), B: acetonitrile (0.01% TFA); Gradient Phase: 5% of B increases to 95% of B within 3 min; Flow Rate: 1.8-2.3 mL/min; Column: SunFire C18, 4.6×50 mm, 3.5 μm; Column Temperature: 50° C. Detectors: ADC ELSD, DAD (214 nm and 254 nm), ES-API. Method B for LC-MS measurement include, as the Instrument: Gilson GX-281; column: Xbridge Prep C18 10 μm OBD, 19×250 mm; Mobile Phase: A: Water (10 mM NH₄HCO₃), B: Acetonitrile; Gradient Phase: 5% to 95% of B within 3 min; Flow Rate: 1.8-2.3 mL/min; Column: XBridge C18, 4.6×50 mm, 3.5 μm; Column Temperature: 50° C. Detectors: ADC ELSD, DAD (214 nm and 254 nm), MSD (ES-API).

Preparative high-pressure liquid chromatography (Prep-HPLC) in an acidic or basic solvent system can be on a Gilson GX-281 instrument. The acidic solvent system includes a Waters SunFire 10 μm C18 column (100 Å, 250×19 mm), and solvent A for prep-HPLC is water/0.05% TFA and solvent B is acetonitrile. The elution conditions can be a linear gradient increase of solvent B from 5% to 100% over a time period of 20 min at a flow rate of 30 mL/min. The basic solvent system includes a Waters Xbridge 10 μm C18 column (100 Å, 250×19 mm), and solvent A for prep-HPLC is water/10 mM ammonium bicarbonate (NH₄HCO₃) and solvent B is acetonitrile. The elution conditions can be a linear gradient increase of solvent B from 5% to 100% over a time period of 20 min at a flow rate of 30 mL/min.

Flash chromatography can be performed on a Biotage instrument, with Agela Flash Column silica-CS cartridges; Reversed phase flash chromatography can be performed on Biotage instrument, with Boston ODS or Agela C18 cartridges.

Analytical chiral HPLC method—SFC conditions

a) Instrument: SFC Method Station (Thar, Waters)

b) Column: CHIRALPAK AD-H/AS-H/OJ-H/OD-H 4.6×100 mm, 5 μm (Daicel)

c) Column temperature: 40° C.

d) Mobile phase: CO₂/IPA (0.1% DEA)=55/45

e) Flow: 4.0 mL/min

f) Back Pressure: 120 Bar

g) Injection volume: 2 μL

Preparative chiral HPLC method—SFC conditions
a) Instrument: SFC-80 (Thar, Waters)
b) Column: CHIRALPAK AD-H/AS-H/OJ-H/OD-H 20×250 mm, 10 μm (Daicel)
c) Column temperature: 35° C.
d) Mobile phase: CO₂/IPA (0.2% Methanol Ammonia)=30/70
e) Flow rate: 80 g/min
f) Back pressure: 100 bar
g) Detection wavelength: 214 nm
h) Cycle time: 6.0 min
i) Sample solution: 1500 mg dissolved in 70 mL Methanol
j) Injection volume: 2 mL (loading: 42.86 mg/injection)

### Preparation Methods

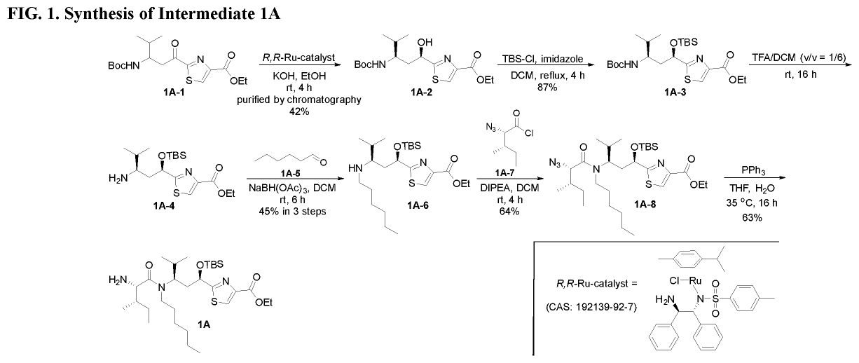

Intermediate 1A was synthesized as in FIG. 1.

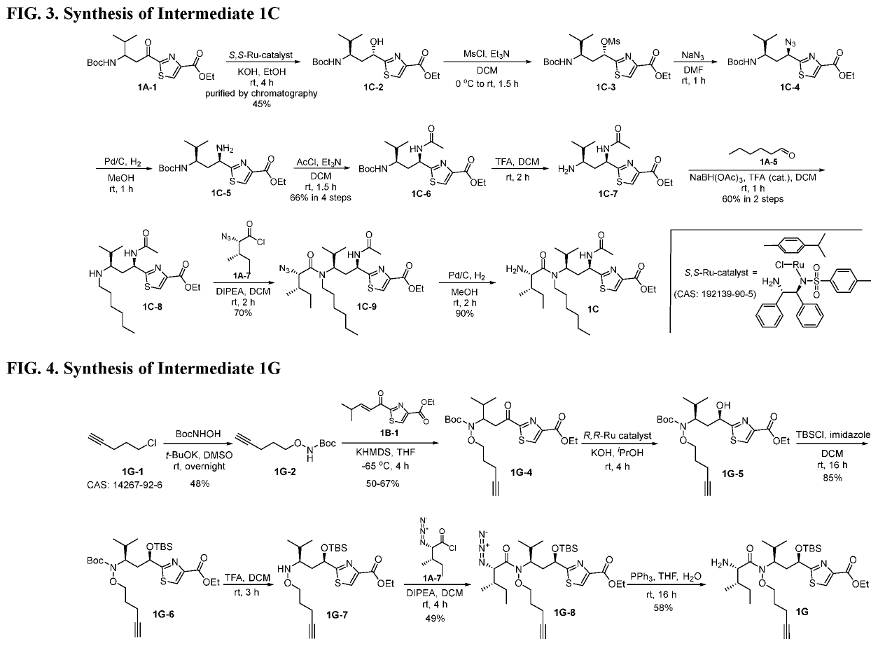

Compound 1A-1 (FIG. 1) was synthesized according to Organic & Biomolecular Chemistry (2013), 11(14), 2273-2287 and compound 1A-7 (FIG. 1) was synthesized according to WO 2008/138561 A1. Stereospecific reduction of ketone 1A-1 using a (R,R)—Ru-catalyst provided (R,R)-isomer 1A-2 (FIG. 1). Stereospecific reduction of ketone 1A-1 using a (S,S)—Ru-catalyst provided (S,R)-isomer 1C-2 (FIG. 3).

### Ethyl 2-[(1R,3R)-3-{[(tert-butoxy)carbonyl]amino}-1-hydroxy-4-methylpentyl]-1,3-thiazole-4-carboxylate (1A-2)

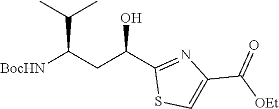 [SMILES: CCOC(=O)c1csc([C@H](O)C[C@@H](NC(=O)OC(C)(C)C)C(C)C)n1]

To a solution of compound 1A-1 (0.30 kg, 0.81 mol) in ethanol (4.5 L) were added R,R—Ru-catalyst (CAS: 192139-92-7, 26 g, 41 mmol) and potassium hydroxide (4.5 g, 81 mmol). After stirring at room temperature for 3 hours, and monitoring by LCMS, the reaction mixture was quenched with sat. aq. ammonium chloride (1.5 L). The volatiles were removed in vacuo and the residue was diluted with water (1.2 L). The aqueous mixture was extracted with ethyl acetate (2.0 L×2) and the combined organic extracts were washed with brine (0.50 L), dried over anhydrous sodium sulfate, and concentrated in vacuo. The crude product was purified by silica gel column chromatography (9-15% ethyl acetate in petroleum ether) to give compound 1A-2 (0.13 kg, 42% yield) as a white solid. ESI m/z: 373 (M+H)+, 395 (M+Na)+. TLC (silica gel): R_{f}=0.4 (33% ethyl acetate in petroleum ether; the R_{f} value for the other diastereoisomer was 0.2.), ¹H NMR (400 MHz, CDCl₃) δ 8.12 (s, 1H), 5.20 (d, J=4.4 Hz, 1H), 5.05-4.97 (m, 1H), 4.55 (d, J=10 Hz, 1H), 4.42 (q, J=7.2 Hz, 2H), 3.81-3.66 (m, 1H), 2.14-2.03 (m, 1H), 1.82-1.69 (m, 2H), 1.44 (s, 9H), 1.40 (t, J=7.2 Hz, 3H), 0.96 (d, J=2.0 Hz, 3H), 0.95 (d, J=2.4 Hz, 3H) ppm. >99.9% ee after chromatography via AS, AD, OD, and OJ columns.

### Ethyl 2-[(1R,3R)-3-{[(tert-butoxy)carbonyl]amino}-1-[(tert-butyldimethylsilyl)oxy]-4-methylpentyl]-1,3-thiazole-4-carboxylate (1A-3)

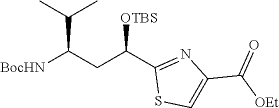 [SMILES: CCOC(=O)c1csc([C@@H](C[C@@H](NC(=O)OC(C)(C)C)C(C)C)O[Si](C)(C)C(C)(C)C)n1]

To a solution of compound 1A-2 (0.11 kg, 0.30 mol) in DCM (1.1 L) under nitrogen was subsequently added imidazole (0.12 kg, 1.8 mol) portionwise and tert-butyldimethylsilyl chloride (TBSCl) (0.14 kg, 0.90 mol) dropwise over 15 minutes. The reaction mixture was refluxed (35° C.) for 4 hours until 1A-2 was totally consumed, according to LCMS. After cooling to room temperature, the reaction mixture was quenched with sat. aq. ammonium chloride (0.40 L) and extracted with DCM (0.40 L×2). The combined organic solution was washed with brine, dried over anhydrous sodium sulfate, and concentrated in vacuo. The residue was dissolved into ethyl acetate (0.40 L) and concentrated in vacuo, which was repeated 10 times to give crude 1A-3 (0.14 kg, crude) as a yellow oil. Crude 1A-3 was used in the next step without further purification. ESI m/z: 487 (M+H)⁺, 509 (M+Na)+. ¹H NMR (400 MHz, CDCl₃) δ 8.09 (s, 1H), 5.18 (dd, J=9.2 and 2.0 Hz, 1H), 4.64 (d, J=9.2 Hz, 1H), 4.41 (q, J=7.2 Hz, 2H), 3.81-3.66 (m, 1H), 1.89-1.77 (m, 2H), 1.71-1.61 (m, 1H), 1.44 (s, 9H), 1.39 (t, J=7.2 Hz, 3H), 0.92 (s, 9H), 0.85-0.81 (m, 6H), 0.13 (s, 3H), −0.10 (s, 3H) ppm.

### Ethyl 2-[(1R,3R)-3-amino-1-[(tert-butyldimethylsilyl)oxy]-4-methylpentyl]-1,3-thiazole-4-carboxylate (1A-4)

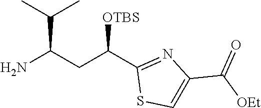 [SMILES: CCOC(=O)c1csc([C@@H](C[C@@H](N)C(C)C)O[Si](C)(C)C(C)(C)C)n1]

A solution of crude 1A-3 (0.14 kg, 0.29 mol) in DCM (1.4 L) was cooled to 0° C. To the cooled solution was added TFA (0.24 L) dropwise over 30 minutes. The resulting mixture was stirred at room temperature for 16 hours until 1A-3 was totally consumed, according to LCMS. The mixture was then cooled to 0° C. and quenched with sat. aq. sodium bicarbonate (2.8 L). The organic layer was washed with water (0.28 L×2) and brine (0.28 L), dried over anhydrous sodium sulfate, and concentrated in vacuo to give crude compound 1A-4 (0.14 kg, crude) as a semi-solid. Crude 1A-4 was used in the next step without further purification. ESI m/z: 387 (M+H)+. ¹H NMR (400 MHz, CDCl₃) δ 8.09 (s, 1H), 5.58-5.53 (m, 1H), 4.37 (q, J=7.2 Hz, 2H), 3.15-3.02 (m, 1H), 2.32-2.20 (m, 1H), 2.16-1.95 (m, 2H), 1.38 (t, J=7.2 Hz, 3H), 0.98-0.95 (m, 6H), 0.94 (s, 9H), 0.20 (s, 3H), 0.06 (s, 3H) ppm.

### Ethyl 2-[(1R,3R)-1-[(tert-butyldimethylsilyl)oxy]-3-(hexylamino)-4-methylpentyl]-1,3-thiazole-4-carboxylate (1A-6)

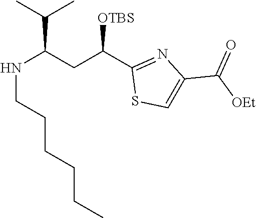 [SMILES: CCCCCCN[C@H](C[C@@H](O[Si](C)(C)C(C)(C)C)c1nc(C(=O)OCC)cs1)C(C)C]

To a solution of crude compound 1A-4 (90 g, 0.23 mol) in DCM (0.12 L) was added hexanal (1A-5, 20 g, 0.20 mol) dropwise over 10 minutes under nitrogen. The reaction mixture was stirred at room temperature for 3 hours before sodium triacetoxyborohydride (0.15 kg, 0.70 mol) was added portionwise into the reaction mixture under nitrogen at 0° C. The reaction mixture was then stirred at room temperature for an hour, and monitored by LCMS. The resulting mixture was quenched with sat. aq. sodium bicarbonate (0.20 L) and diluted with water (0.20 L). The organic layer was washed with water (0.20 L) and brine (0.20 L), dried over anhydrous sodium sulfate, and concentrated in vacuo. The residue was purified by silica gel column chromatography (9-50% ethyl acetate in petroleum ether) to give compound 1A-6 (45 g, 41% yield in 3 steps) as a white solid. ESI m/z: 471 (M+H)⁺. ¹H NMR (400 MHz, CDCl₃) δ 8.12 (s, 1H), 5.27 (t, J=5.6 Hz, 1H), 4.46-4.36 (m, 2H), 3.00-2.87 (m, 2H), 2.80-2.68 (m, 1H), 2.20-2.06 (m, 3H), 1.75-1.62 (m, 1H), 1.40 (t, J=7.2 Hz, 3H), 1.34-1.21 (m, 8H), 0.94 (s, 9H), 0.93-0.85 (m, 9H), 0.20 (s, 3H), 0.06 (s, 3H) ppm.

### Ethyl 2-[(1R,3R)-3-[(2S,3S)-2-azido-N-hexyl-3-methylpentanamido]-1-[(tert-butyldimethylsilyl)oxy]-4-methylpentyl]-1,3-thiazole-4-carboxylate (1A-8)

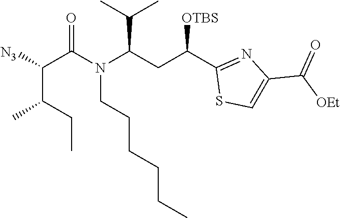 [SMILES: CCCCCCN(C(=O)[C@@H](N=[N+]=[N-])[C@@H](C)CC)[C@H](C[C@@H](O[Si](C)(C)C(C)(C)C)c1nc(C(=O)OCC)cs1)C(C)C]

To a cooled solution of compound 1A-6 (6.0 g, 13 mmol) in DCM (60 mL) at 0° C. was subsequently added DIPEA (8.2 g, 64 mmol) dropwise over 2 minutes and compound 1A-7 (7.9 g, 45 mmol) dropwise over 5 minutes under nitrogen. The reaction mixture was slowly warmed to room temperature and was allowed to stir for an hour until 1A-6 was totally consumed, according to LCMS. To the resulting mixture was added brine (12 mL). The aqueous layer was extracted with DCM (18 mL), and the combined DCM solution was dried over anhydrous sodium sulfate and concentrated in vacuo. The crude product was purified by silica gel column chromatography (10% ethyl acetate in petroleum ether) to give compound 1A-8 (5.0 g, 64% yield) as a yellow oil. ESI m/z: 610 (M+H)⁺, 632 (M+Na)⁺. ¹H NMR (400 MHz, CDCl₃) δ 8.10 (s, 1H), 4.99-4.91 (m, 1H), 4.47-4.32 (m, 3H), 3.32-3.16 (m, 2H), 2.88-3.02 (m, 1H), 2.29-2.19 (m, 1H), 2.10-2.06 (m, 1H), 1.88-1.73 (m, 2H), 1.39 (t, J=7.2 Hz, 3H), 1.35-1.20 (m, 10H), 1.03-0.95 (m, 6H), 0.94 (s, 9H), 0.93-0.85 (m, 9H), 0.16 (s, 3H), −0.10 (s, 3H) ppm. Optical Rotation: +99.5° (Temperature: 19.8° C., concentration: 1.25 mg/mL in methanol).

### Ethyl 2-[(1R,3R)-3-[(2S,3S)-2-amino-N-hexyl-3-methylpentanamido]-1-[(tert-butyldimethylsilyl)oxy]-4-methylpentyl]-1,3-thiazole-4-carboxylate (1A)

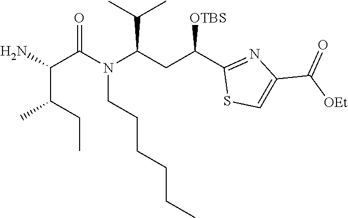 [SMILES: CCCCCCN(C(=O)[C@@H](N)[C@@H](C)CC)[C@H](C[C@@H](O[Si](C)(C)C(C)(C)C)c1nc(C(=O)OCC)cs1)C(C)C]

To a solution of compound 1A-8 (5.0 g, 8.2 mmol) in THE (50 mL) and water (2.5 mL) was added triphenylphosphine (15 g, 57 mmol) dropwise over 5 minutes at room temperature under nitrogen. The reaction mixture was stirred at 35° C. for 16 hours, and monitored by LCMS. The volatiles were then removed in vacuo and the residue was dissolved in ethyl acetate (10 mL). To the mixture was added zinc chloride (3.3 g, 25 mmol), and the suspension was stirred at room temperature for 2 hours. The resulting suspension was filtered and the filtrate was concentrated in vacuo. The residue was purified by silica gel column chromatography (50% ethyl acetate in petroleum ether) to give intermediate 1A (3.0 g, 63% yield) as a yellow solid. ESI m/z: 584 (M+H)⁺. ¹H NMR (400 MHz, CDCl₃) δ 8.50 (s, 1H), 4.86-4.77 (m, 1H), 4.39-4.23 (m, 2H), 3.74-3.64 (m, 1H), 3.29-3.16 (m, 1H), 3.12-2.99 (m, 2H), 2.19-2.03 (m, 2H), 1.98-1.88 (m, 1H), 1.86-1.74 (m, 1H), 1.68-1.54 (m, 2H), 1.32 (t, J=7.2 Hz, 3H), 1.35-1.20 (m, 10H), 1.03-0.94 (m, 6H), 0.90 (s, 9H), 0.88-0.77 (m, 9H), 0.13 (s, 3H), −0.11 (s, 3H) ppm. Optical Rotation: +41.3° (Temperature: 19.8° C., concentration: 1.16 mg/mL in methanol).

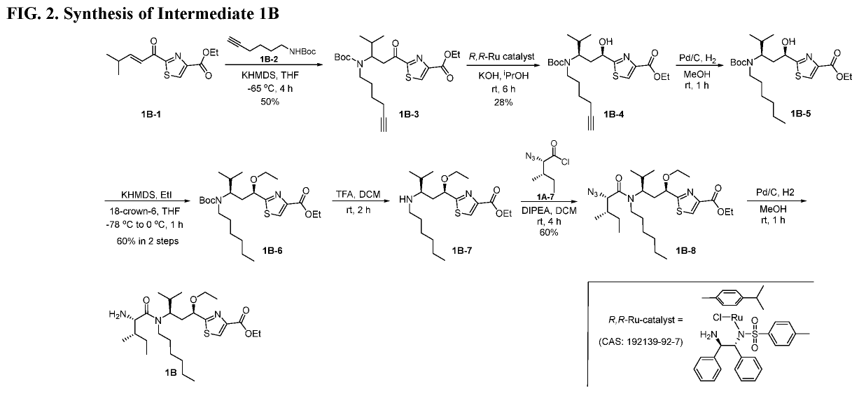

Intermediate 1B was synthesized as in FIG. 2.

Compound 1B-1 was synthesized according to WO 2008/138561 A1.

### Ethyl 2-(3-{[(tert-butoxy)carbonyl](hex-5-yn-1-yl)amino}-4-methylpentanoyl)-1,3-thiazole-4-carboxylate (1B-3)

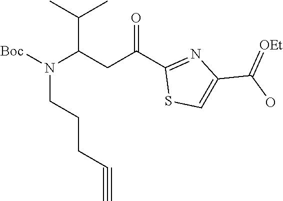

To a −65° C. solution of compound 1B-2 (73 g, 0.37 mol) in dry THE (1.2 L) was subsequently added dropwise KHMIDS (1 M in THF, 0.37 L, 0.37 mol) over 30 minutes followed by a solution of compound 1B-1 (62 g, 0.25 mol) in THE (0.20 L) over 30 minutes keeping the temperature below −60° C. The reaction mixture was stirred at −65° C. for 4 hours until 1B-1 was totally consumed, according to TLC. The resulting mixture was quenched with sat. aq. ammonium chloride (0.30 L). The aqueous layer was extracted ethyl acetate (0.5 L×3). All the organics were combined and washed with brine (0.5 L), dried over anhydrous sodium sulfate, and concentrated in vacuo. The residue was purified by silica gel column chromatography (10% ethyl acetate in petroleum ether) to give compound 1B-3 (55 g, 50% yield) as a yellow oil. ESI m/z: 351 (M−Boc+H)⁺. ¹H NMR (400 MHz, CDCl₃) δ 8.42 (s, 1H), 4.44 (q, J=7.2 Hz, 2H), 4.09 (br s, 1H), 3.70-3.42 (m, 2H), 3.30-2.99 (m, 2H), 2.25-2.15 (m, 2H), 2.12-1.90 (m, 2H), 1.70-1.55 (m, 2H), 1.55-1.43 (m, 5H), 1.42 (s, 9H), 1.00 (d, J=6.6 Hz, 3H), 0.93 (d, J=6.6 Hz, 3H) ppm.

### Ethyl 2-[(1R,3R)-3-{[(tert-butoxy)carbonyl](hex-5-yn-1-yl)amino}-1-hydroxy-4-methylpentyl]-1,3-thiazole-4-carboxylate (1B-4)

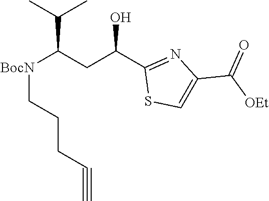 [SMILES: C#CCCCN(C(=O)OC(C)(C)C)[C@H](C[C@@H](O)c1nc(C(=O)OCC)cs1)C(C)C]

To a solution of compound 1B-3 (54 g, 0.12 mol) in isopropanol (0.60 L) were added R,R—Ru-catalyst (CAS: 192139-92-7, 3.9 g, 6.0 mmol) and potassium hydroxide (0.73 g, 12 mmol). After stirring at room temperature for 6 hours until 1B-3 was totally consumed, according to TLC, the reaction mixture was quenched with sat. aq. ammonium chloride (0.3 L). The mixture was extracted with ethyl acetate (0.5 L×3) and the combined organic extracts were washed with brine (0.5 L), dried over anhydrous sodium sulfate, and concentrated in vacuo. The crude product was purified by silica gel column chromatography (10-20% ethyl acetate in petroleum ether) to give compound 1B-4 (15 g, 28% yield) as yellow oil. ESI m/z: 453 (M+H)⁺, 475 (M+Na)⁺.

### Ethyl 2-[(1R,3R)-3-{[(tert-butoxy)carbonyl](hexyl)amino}-1-hydroxy-4-methylpentyl]-1,3-thiazole-4-carboxylate (1R-5)

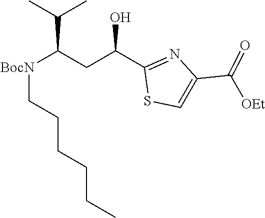 [SMILES: CCCCCCN(C(=O)OC(C)(C)C)[C@H](C[C@@H](O)c1nc(C(=O)OCC)cs1)C(C)C]

To a solution of compound 1B-4 (0.45 g, 1.0 mmol) in methanol (10 mL) was added 10% Palladium on carbon (50 mg, 11 wt %) under nitrogen. The suspension was degassed and purged with hydrogen 3 times, and was then stirred at room temperature under a hydrogen balloon for an hour. The reaction was monitored by LCMS. The resulting suspension was filtered through Celite and the filtrate was concentrated in vacuo to give crude product 1B-5 (0.45 g, crude) as a white solid. Crude 1B-5 was used in the next step without further purification. ESI m/z: 457 (M+H)⁺, 479 (M+Na)⁺.

### Ethyl 2-[(1R,3R)-3-{[(tert-butoxy)carbonyl](hexyl)amino}-1-ethoxy-4-methylpentyl]-1,3-thiazole-4-carboxylate (1B-6)

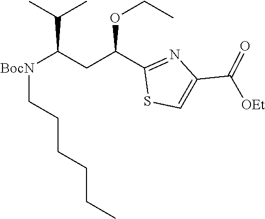 [SMILES: CCCCCCN(C(=O)OC(C)(C)C)[C@H](C[C@@H](OCC)c1nc(C(=O)OCC)cs1)C(C)C]

To a solution of compound 1B-5 (0.44 g, 1.0 mmol) and 18-crown-6 (0.53 g, 2.0 mmol) in THE (10 mL) was added a solution of KHMDS in THE (1.0 M, 2.0 mL, 2.0 mmol) dropwise over 5 minutes at −78° C. under nitrogen. The reaction mixture was stirred at −78° C. for 30 minutes before the addition of ethyliodide (0.78 g, 5.0 mmol). The mixture was then slowly warmed to room temperature, stirred for an hour, and monitored by LCMS. After cooling to −10° C., the resulting mixture was quenched by water (20 mL) and extracted with ethyl acetate (20 mL×3). The combined organic solution was washed with brine (20 mL), dried over anhydrous sodium sulfate, and concentrated in vacuo. The crude product was purified by prep-HPLC (5-95% acetonitrile in aq. ammonium bicarbonate (10 mM)) to give compound 1B-6 (0.29 g, 60% yield in 2 steps) as a white solid. ESI m/z: 485 (M+H), 507 (M+Na)⁺.

### Ethyl 2-[(1R,3R)-1-ethoxy-3-(hexylamino)-4-methylpentyl]-1,3-thiazole-4-carboxylate (1B-7)

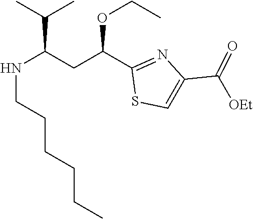 [SMILES: CCCCCCN[C@H](C[C@@H](OCC)c1nc(C(=O)OCC)cs1)C(C)C]

To a solution of compound 1B-6 (0.20 g, 0.41 mmol) in DCM (5.0 mL) was added TFA (1.0 mL) dropwise at room temperature. The mixture was stirred at room temperature for 2 hours until Boc was totally removed according to LCMS. The volatiles were removed in vacuo to provide crude product 1B-7 (0.12 g, crude) as a white solid. Crude 1B-7 was used in the next step without further purification. ESI m/z: 385 (M+H)⁺.

### Ethyl 2-[(1R,3R)-3-[(2S,3S)-2-azido-N-hexyl-3-methylpentanamido]-1-ethoxy-4-methylpentyl]-1,3-thiazole-4-carboxylate (1B-8)

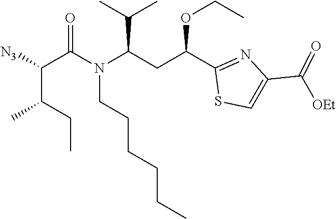 [SMILES: CCCCCCN(C(=O)[C@@H](N=[N+]=[N-])[C@@H](C)CC)[C@H](C[C@@H](OCC)c1nc(C(=O)OCC)cs1)C(C)C]

Following a similar procedure for 1A-8 except using 1B-6 (0.15 g, 0.39 mmol) instead of 1A-6, compound 1B-8 (0.12 g, 60% yield) was obtained as a white solid. ESI m/z: 520 (M+H)⁺, 542 (M+Na)⁺.

### Ethyl 2-[(1R,3R)-3-[(2S,3S)-2-amino-N-hexyl-3-methylpentanamido]-1-ethoxy-4-methylpentyl]-1,3-thiazole-4-carboxylate (1B)

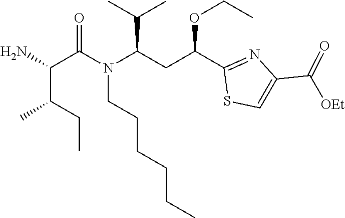 [SMILES: CCCCCCN(C(=O)[C@@H](N)[C@@H](C)CC)[C@H](C[C@@H](OCC)c1nc(C(=O)OCC)cs1)C(C)C]

To a solution of compound 1B-8 (0.10 g, 0.19 mmol) in methanol (10 mL) was added 10% Palladium on carbon (50 mg, 50 wt %) under nitrogen. The suspension was degassed and purged with hydrogen 3 times. The reaction was then stirred at room temperature under a hydrogen balloon for an hour, and monitored by LCMS. The resulting suspension was filtered through Celite and the filtrate was concentrated in vacuo to give intermediate 1B (0.16 g, 85% yield) as a white solid. Intermediate 1B was used in the next step without purification. ESI m/z: 498 (M+H)⁺.

Intermediate 1C was synthesized as in FIG. 3.

### Ethyl 2-[(1S,3R)-3-{[(tert-butoxy)carbonyl]amino}-1-hydroxy-4-methylpentyl]-1,3-thiazole-4-carboxylate (1C-2)

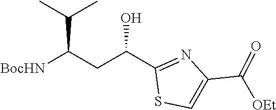 [SMILES: CCOC(=O)c1csc([C@@H](O)C[C@@H](NC(=O)OC(C)(C)C)C(C)C)n1]

Following a similar procedure for 1A-2 except using S,S—Ru-catalyst (CAS: 192139-90-5) instead of R,R—Ru-catalyst, compound 1C-2 (1.7 g, 45% yield, 80e.e %.) was obtained as a colorless oil. ESI m/z: 373 (M+H)⁺. TLC (silica gel): R_{f}=0.3 (33% ethyl acetate in petroleum ether; the R_{f} value for the other diastereoisomer was 0.4.).

A small amount of the product was separated by chiral-HPLC (Column: R′R WHELK 20*250 mm, 10 μm (Daicel), Mobile phase: CO₂/MeOH (0.2% methanol ammonia)=90/10) to give enantiopure product 1C-2 (>99.9% ee). Chiral HPLC: >99.9% using an AS, AD, OD, and OJ column. ¹H NMR (400 MHz, CDCl₃) δ 8.42 (s, 1H), 6.53 (d, J=9.3 Hz, 1H), 6.25 (d, J=4.7 Hz, 1H), 4.81 (d, J=4.8 Hz, 1H), 4.30-4.27 (m, 2H), 3.53 (s, 1H), 2.06-1.89 (m, 1H), 1.77-1.70 (m, 2H), 1.34 (s, 9H), 1.30 (t, J=7.2 Hz, 3H), 0.81 (d, J=3.4 Hz, 3H), 0.78 (d, J=3.4 Hz, 3H) ppm.

### Ethyl 2-[(1S,3R)-3-{[(tert-butoxy)carbonyl]amino}-1-(methanesulfonyloxy)-4-methylpentyl]-1,3-thiazole-4-carboxylate (1C-3)

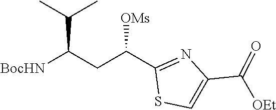 [SMILES: CCOC(=O)c1csc([C@H](C[C@@H](NC(=O)OC(C)(C)C)C(C)C)OS(C)(=O)=O)n1]

To a suspension of compound 1C-2 (1.4 g, 4.0 mmol, 80% ee) in DCM (50 mL) was subsequently added triethylamine (0.60 g, 6.0 mmol) and methanesulfonyl chloride (0.55 g, 4.8 mmol) dropwise at 0° C. After the reaction turned clear, the reaction mixture was stirred at 0° C. for an hour, then at room temperature for 30 minutes, and monitored by TLC. The solution was successively washed with aq. hydrochloride (1 N, 50 mL), water (50 mL), aq. sodium carbonate (10%, 50 mL), and brine (50 mL). The resulting organic solution was dried over anhydrous sodium sulfate and concentrated in vacuo to give crude compound 1C-3 (1.6 g, crude) as a yellow oil. Crude 1C-3 was used in the next step without further purification. ESI m/z: 451 (M+H)⁺.

### Ethyl 2-[(1R,3R)-1-azido-3-{[(tert-butoxy)carbonyl]amino}-4-methylpentyl]-1,3-thiazole-4-carboxylate (1C-4)

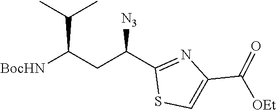 [SMILES: CCOC(=O)c1csc([C@@H](C[C@@H](NC(=O)OC(C)(C)C)C(C)C)N=[N+]=[N-])n1]

To a stirred mixture of compound 1C-3 (1.6 g, crude) in DMF (10 mL) was added sodium azide (1.2 g, 18 mmol) at room temperature. The reaction mixture was stirred at room temperature for an hour, and monitored by LCMS. The mixture was then diluted with water (50 mL) and extracted with ethyl acetate (50 mL×3). The combined organic solution was washed with water (50 mL) and brine (50 mL), dried over anhydrous sodium sulfate, and concentrated in vacuo to give crude compound 1C-4 (1.3 g, crude) as a yellow oil. ESI m/z: 398 (M+H)⁺.

### Ethyl 2-[(1R,3R)-1-amino-3-{[(tert-butoxy)carbonyl]amino}-4-methylpentyl]-1,3-thiazole-4-carboxylate (1C-5)

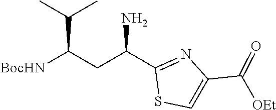 [SMILES: CCOC(=O)c1csc([C@H](N)C[C@@H](NC(=O)OC(C)(C)C)C(C)C)n1]

To a solution of compound 1C-4 (1.3 g, crude) in methanol (50 mL) was added 10% Palladium on carbon (0.12 g, 10 wt %) under nitrogen. The suspension was degassed and purged with hydrogen 3 times. The reaction was then stirred at room temperature under a hydrogen balloon for an hour, and monitored by LCMS. The resulting suspension was filtered through Celite and the filtrate was concentrated in vacuo to give crude compound 1C-5 (1.0 g, crude) as a yellow oil. Crude 1C-5 was used in the next step without further purification. ESI m/z: 371 (M+H)⁺.

### Ethyl 2-[(1R,3R)-3-{[(tert-butoxy)carbonyl]amino}-1-acetamido-4-methylpentyl]-1,3-thiazole-4-carboxylate (1C-6)

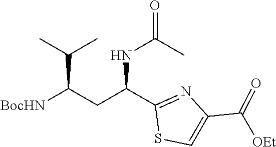 [SMILES: CCOC(=O)c1csc([C@@H](C[C@@H](NC(=O)OC(C)(C)C)C(C)C)NC(C)=O)n1]

To a stirred suspension of compound 1C-5 (1.0 g, crude) in DCM (50 mL) was subsequently added triethylamine (0.45 g, 4.5 mmol) and acetylchloride (0.28 g, 3.6 mmol) at 0° C. After the reaction turned clear, the reaction mixture was stirred at room temperature for 1.5 hours, and monitored by LCMS. The resulting solution was then washed with aq. hydrochloride (1 N, 50 mL), water (50 mL), aq. sodium carbonate (10%, 50 mL), brine (50 mL), dried over anhydrous sodium sulfate, and concentrated in vacuo. The residue was purified by silica gel column chromatography (15-20% ethyl acetate in petroleum ether) to give compound 1C-6 (1.0 g, 66% yield in 4 steps) as a yellow oil. ESI m/z: 413 (M+H)⁺.

### Ethyl 2-[(1R,3R)-3-amino-1-acetamido-4-methylpentyl]-1,3-thiazole-4-carboxylate (1C-7)

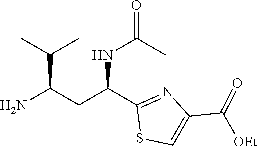 [SMILES: CCOC(=O)c1csc([C@@H](C[C@@H](N)C(C)C)NC(C)=O)n1]

To a solution of compound 1C-6 (1.3 g, 3.0 mmol) in DCM (20 mL) was added TFA (4 mL) at 0° C. The mixture was stirred at room temperature for an hour, and monitored by LCMS. The volatiles were removed in vacuo to give crude compound 1C-7 (1.0 g, crude) as a yellow solid. Crude 1C-7 was used in the next step without further purification. ESI m/z: 314 (M+H)⁺.

### Ethyl 2-[(1R,3R)-1-acetamido-3-(hexylamino)-4-methylpentyl]-1,3-thiazole-4-carboxylate (1C-8)

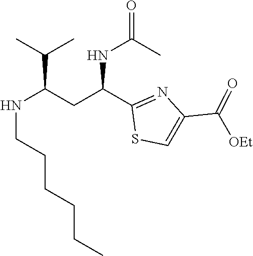 [SMILES: CCCCCCN[C@H](C[C@@H](NC(C)=O)c1nc(C(=O)OCC)cs1)C(C)C]

To a solution of crude compound 1C-7 (0.70 g, 2.2 mmol) in DCM (30 mL) under nitrogen was subsequently added hexanal (1A-5, 0.26 g, 2.6 mmol) dropwise over 5 minutes, sodium triacetoxyborohydride (0.70 g, 3.3 mmol), and 2 drops of TFA. The reaction mixture was stirred at room temperature for an hour, and monitored by LCMS. The resulting mixture was washed with water (20 mL), aq. sodium carbonate (10%, 20 mL), brine (20 mL), dried over anhydrous sodium sulfate, and concentrated in vacuo. The residue was purified by chiral-HPLC (Column: IG 20*250 mm, 10 μm, Mobile phase: CO₂/methanol (0.2% methanol ammonia)=80/20) to give compound 1C-8 (0.52 g, 60% yield in 2 steps) as a colorless oil. ESI m/z: 398 (M+H)⁺. ¹H NMR (400 MHz, DMSO_{d6}) δ 8.77 (d, J=7.8 Hz, 1H), 8.39 (s, 1H), 5.33-5.26 (m, 1H), 4.38-4.18 (m, 2H), 2.56-2.50 (m, 1H), 2.39-2.30 (m, 2H), 1.89 (s, 3H), 1.83-1.70 (m, 2H), 1.37-1.19 (m, 12H), 0.85-0.79 (m, 9H) ppm. >99.9% ee using IG columns.

### Ethyl 2-[(1R,3R)-3-[(2S,3S)-2-azido-N-hexyl-3-methylpentanamido]-1-acetamido-4-methylpentyl]-1,3-thiazole-4-carboxylate (1C-9)

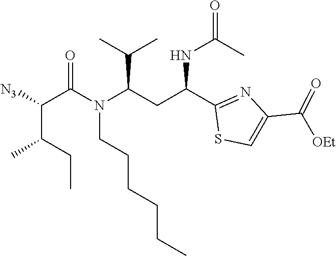 [SMILES: CCCCCCN(C(=O)[C@@H](N=[N+]=[N-])[C@@H](C)CC)[C@H](C[C@@H](NC(C)=O)c1nc(C(=O)OCC)cs1)C(C)C]

To a mixture of compound 1C-8 (0.20 g, 0.50 mmol) in DCM (5 mL) was subsequently added DIPEA (0.13 g, 1.0 mmol) and compound 1A-7 (0.18 g, 1.0 mmol). The mixture was stirred at room temperature for 2 hours, and monitored by LCMS. The volatiles were removed in vacuo and the residue was purified by silica gel column chromatography (15-20% ethyl acetate in petroleum ether) to give compound 1C-9 (0.19 g, 70% yield) as a yellow oil. ESI m/z: 537 (M+H)⁺.

### Ethyl 2-[(1R,3R)-3-[(2S,3S)-2-amino-N-hexyl-3-methylpentanamido]-1-acetamido-4-methylpentyl]-1,3-thiazole-4-carboxylate (1C)

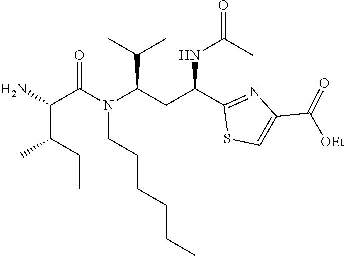 [SMILES: CCCCCCN(C(=O)[C@@H](N)[C@@H](C)CC)[C@H](C[C@@H](NC(C)=O)c1nc(C(=O)OCC)cs1)C(C)C]

To a solution of compound 1C-9 (0.19 g, 0.35 mmol) in methanol (10 mL) was added 10% Palladium on carbon (20 mg, 10 wt %) under nitrogen. The suspension was degassed and purged with hydrogen 3 times. The reaction was then stirred at room temperature under a hydrogen balloon for 2 hours, and monitored by LCMS. The resulting suspension was filtered through Celite and the filtrate was concentrated in vacuo. The residue was purified by silica gel column chromatography (50% ethyl acetate in petroleum ether) to give intermediate 1C (0.15 g, 90% yield) as a yellow oil. ESI m/z: 511 (M+H)⁺.

Intermediate 1G was synthesized as in FIG. 4, and as shown in U.S. patent application Ser. No. 16/724,164, filed Dec. 20, 2019. The synthesis of the corresponding compound from U.S. patent application Ser. No. 16/724,164 is incorporated herein by reference.

Intermediates: MEP

Intermediates MEPa-e were commercially available. CAS numbers and structures appear below.

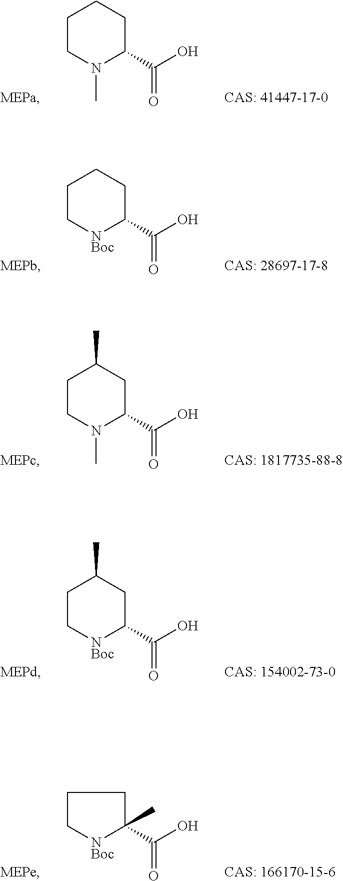

Intermediates: TUP

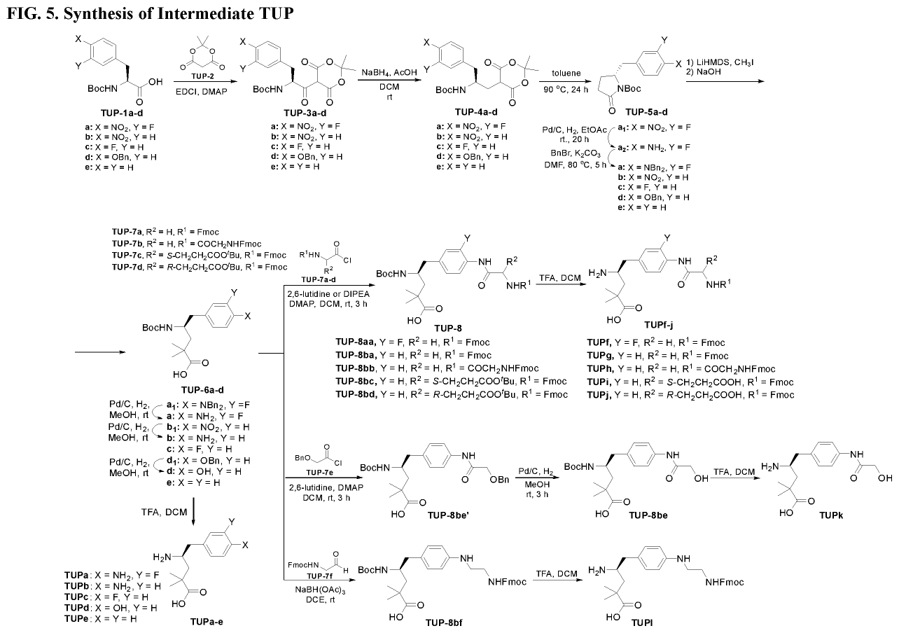

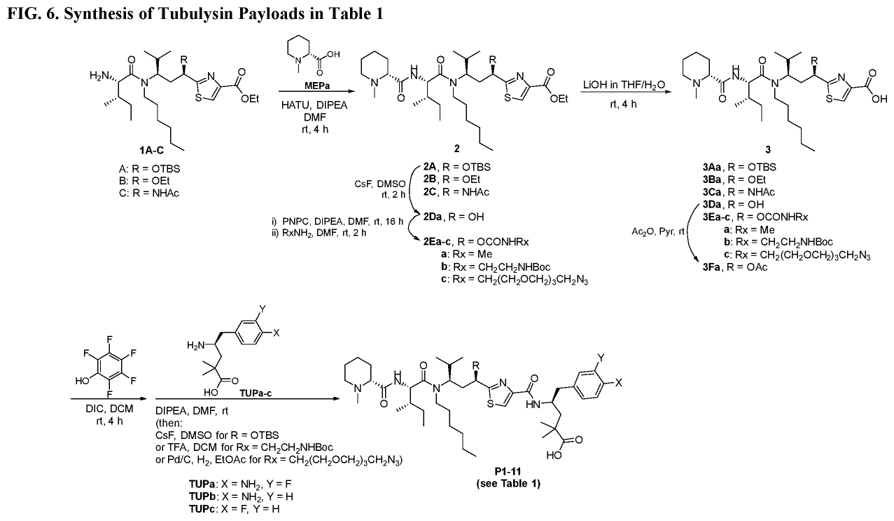

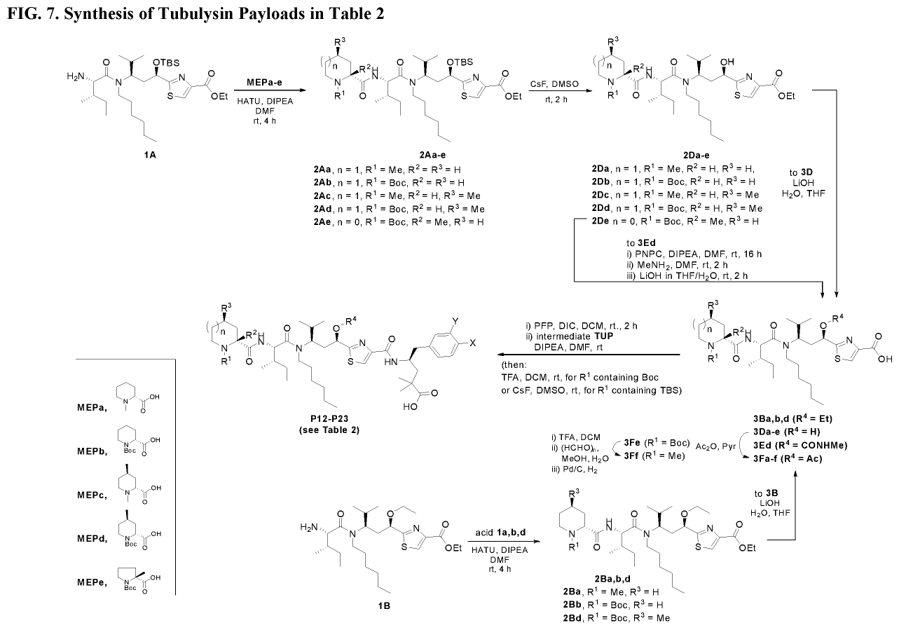

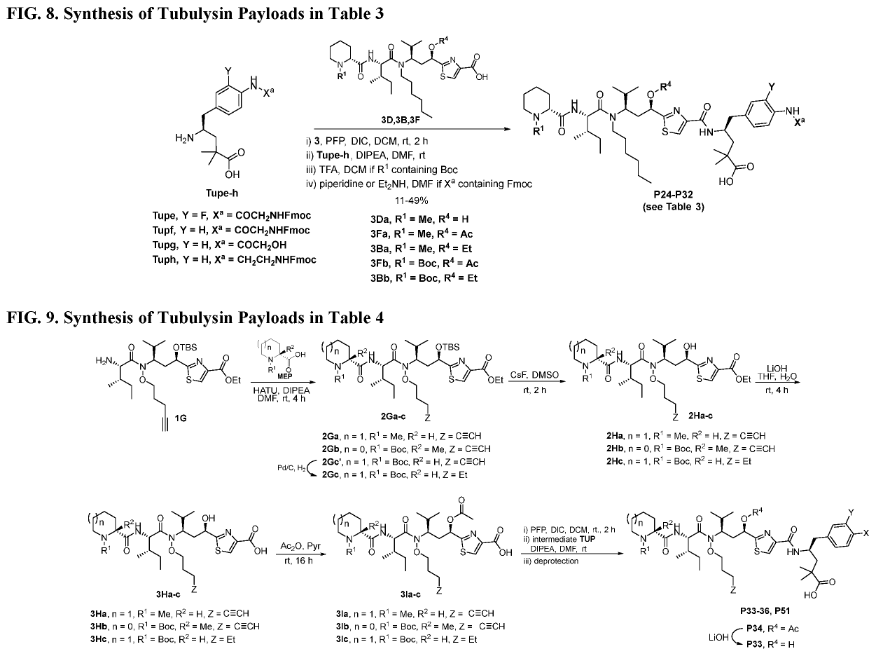

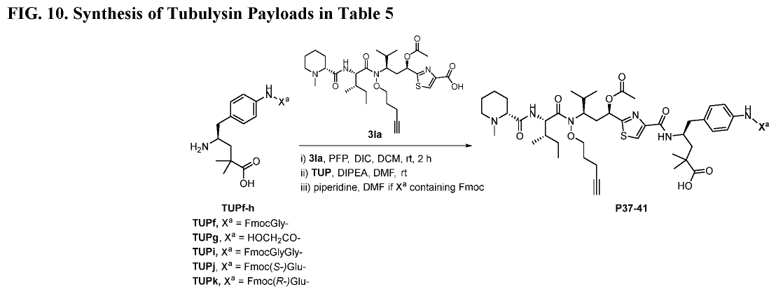

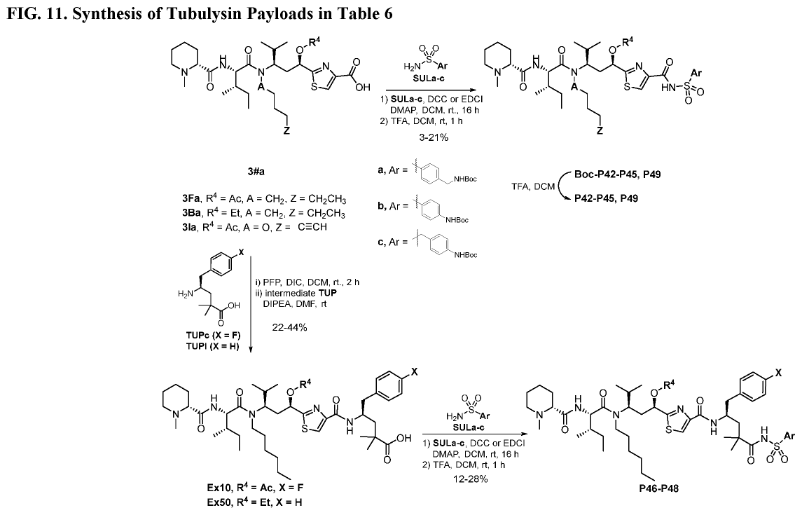

Intermediates TUPa-1 were synthesized as in FIG. 5. Intermediates TUPa-e were synthesized as in U.S. patent application Ser. No. 16/724,164, filed Dec. 20, 2019. The syntheses of the corresponding compounds from U.S. patent application Ser. No. 16/724,164 are incorporated herein by reference. Intermediates TUPf-1 were synthesized following the procedures below.

### (4S)-4-Amino-5-[4-(2-{[(9H-fluoren-9-ylmethoxy)carbonyl]amino}acetamido)-3-fluorophenyl]-2,2-dimethylpentanoic Acid (TUPf)

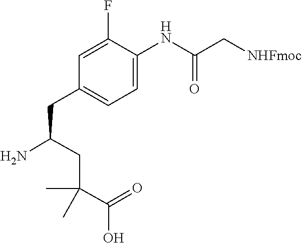

To a solution of Fmoc-Gly-OH (0.25 g, 0.85 mmol) in DCM (10 mL) was added oxalyl chloride (0.16 g, 1.3 mmol) and a drop of DMF. The reaction mixture was stirred at room temperature for an hour, and monitored by LCMS. The volatiles were removed in vacuo and the residue was dissolved in DMF (4 mL). To the solution were added TUP-6a (30 mg, 85 mol) and DIPEA (0.11 g, 0.85 mmol). The reaction mixture was stirred at room temperature for an hour, and monitored by LCMS. The resulting mixture was directly purified by reversed phase flash chromatography (0-100% acetonitrile in aq. TFA (0.01%)) to give compound TUP-8aa (45 mg, 84% yield) as a white solid. ESI m/z: 656 (M+Na)⁺, 534 (M−Boc+H)⁺.

To a solution of compound TUP-8aa (45 mg, 71 μmol) in DCM (0.6 mL) was added TFA (0.2 mL). The reaction mixture was stirred at RT for 3 hours, and monitored by LCMS. The volatiles were removed in vacuo and the residue was purified by reversed phase flash chromatography (0-30% acetonitrile in aq. ammonium bicarbonate (10 mM)) to give TUPf (36 mg, 94% yield) as a white solid. ESI m/z: 534 (M+H)⁺. ¹H NMR (400 MHz, DMSO_{d6}) δ 9.77 (s, 1H), 7.90 (d, J=7.6 Hz, 2H), 7.74-7.71 (m, 3H), 7.67 (t, J=6.0 Hz, 1H), 7.43 (t, J=7.6 Hz, 2H), 7.34 (t, J=7.2 Hz, 2H), 7.20 (d, J=10.4 Hz, 1H), 7.05 (t, J=8.4 Hz, 1H), 4.33-4.29 (m, 2H), 4.25 (d, J=6.4 Hz, 1H), 3.86 (d, J=5.6 Hz, 2H), 3.44-3.39 (m, 3H), 2.78 (d, J=6.4 Hz, 2H), 1.77-1.74 (m, 2H), 1.10 (s, 3H), 1.07 (s, 3H) ppm.

### (4S)-4-Amino-5-[4-(2-{[(9H-fluoren-9-ylmethoxy)carbonyl]amino}acetamido)phenyl]-2,2-dimethylpentanoic Acid (TUPg)

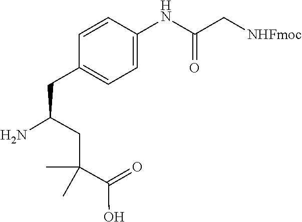

To a solution of TUP-6b (0.34 g, 1.0 mmol) in DCM (5.0 mL) was added 2,6-lutidine (21 mg, 2.0 mmol), DMAP (12 mg, 0.10 mmol) and Fmoc-Gly-Cl (TUP-7a) (0.38 g, 1.2 mmol). The reaction mixture was stirred at room temperature for 3 hours, and monitored by LCMS. The resulting mixture was diluted with ethyl acetate (50 mL), washed with water and brine, dried over anhydrous sodium sulfate, and concentrated in vacuo. The residue was purified by reversed phase flash chromatography (0-100% acetonitrile in aq. TFA (0.3%)) to give compound TUP-8ba (0.28 g, 45% yield) as a white solid. ESI m/z 516 (M−Boc+H)⁺.

To a solution of TUP-8ba (61 mg, 0.10 mmol) in DCM (5 mL) was added TFA (1.0 mL). The mixture was stirred at room temperature for 2 hours until Boc was totally removed in vacuo, according to LCMS. The volatiles were removed in vacuo to give crude product TUPg (51 mg, >100% crude yield) as a white solid. ESI m/z 516 (M+H)⁺.

### (4S)-4-Amino-5-{4-[2-(2-{[(9H-fluoren-9-ylmethoxy)carbonyl]amino}acetamido)acetamido]phenyl}-2,2-dimethylpentanoic Acid (TUPh)

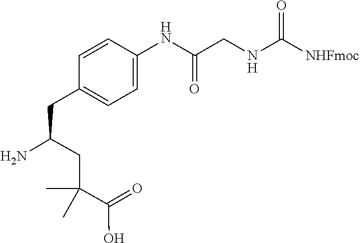

To a solution of Fmoc-Gly-Gly-OH (0.30 g, 0.85 mmol) in dry DCM (10 mL) was added oxalyl chloride (0.17 g, 1.3 mmol) and DMF (3 mg, 43 μmol). The reaction mixture was stirred at room temperature for half an hour, and monitored by LCMS and TLC (10% methanol in DCM). The volatiles were removed in vacuo and the residue was added to a solution of TUP-6b (0.34 g, 1.0 mmol) in dry DMF (5 mL). To the stirred reaction mixture was added DIPEA (0.33 g, 2.6 mmol) dropwise. The mixture was stirred at room temperature for 3 hours, and monitored by LCMS. The resulting mixture was directly purified by reversed phase flash chromatography (0-30% acetonitrile in aq. ammonium bicarbonate (10 mM)) to give TUP-8bb (0.15 g) as a white solid. ESI m/z: 695 (M+Na)⁺.

To a solution of TUP-8bb (0.15 g) in DCM (6 mL) was added TFA (2 mL), and the reaction mixture was stirred at room temperature for 3 hours until Boc was totally removed according to LCMS. The resulting mixture was concentrated in vacuo and the residue was purified by reversed phase flash chromatography (0-30% acetonitrile in aq. TFA (0.01%)) to give intermediate TUPh (80 mg, 14% yield from TUP-6b) as a white solid. ESI m/z: 573 (M+H)⁺.

### (4S)-4-Amino-5-{4-[(2S)-4-carboxy-2-{[(9H-fluoren-9-ylmethoxy)carbonyl]amino}butanamido]phenyl}-2,2-dimethylpentanoic Acid (TUPi)

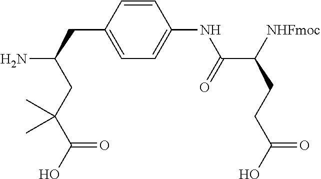

To a solution of Fmoc-Glu(O^{t}Bu)-OH (0.16 g, 0.37 mmol) in dry DCM (6 mL) was added oxalyl chloride (0.15 g, 1.2 mmol) at 0° C. The mixture was stirred at room temperature for an hour, and monitored by LCMS. The volatiles were removed in vacuo to provide crude Fmoc-Glu(O^{t}Bu)-Cl (0.16 g), which was used in the next step without further purification.

To a mixture of TUP-6b (66 mg, 0.20 mmol) and DIPEA (52 mg, 0.40 mmol) in DMF (2 mL) was added crude Fmoc-Glu(O^{t}Bu)-Cl (0.13 g). The reaction mixture was stirred at room temperature for 2 hours, and monitored by LCMS. The resulting mixture was purified directly by flash chromatography (0-10% methanol in DCM) to give TUP-8bc (0.20 g) as a yellow oil. ESI m/z: 766 (M+Na)⁺.

To a solution of TUP-8bc (0.18 g) in DCM (4 mL) was added TFA (1 mL). The reaction mixture was stirred at room temperature for an hour, and monitored by LCMS. The volatiles were removed in vacuo to give TUPi (0.14 g, >100% crude yield, TFA salt) as a yellow solid. ESI m/z: 588 (M+H)⁺.

### (4S)-4-Amino-5-{4-[(2R)-4-carboxy-2-{[(9H-fluoren-9-ylmethoxy)carbonyl]amino}butanamido]phenyl}-2,2-dimethylpentanoic Acid (TUPj)

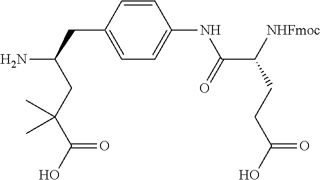

Following a similar procedure for TUPi except starting from Fmoc-D-Glu(O^{t}Bu)-OH, TUPj (0.13 g, >100% crude yield, TFA salt) was obtained as a yellow solid. ESI m/z: 588 (M+H)⁺.

### (4S)-4-Amino-5-[4-(2-hydroxyacetamido)phenyl]-2,2-dimethylpentanoic Acid (TUPk)

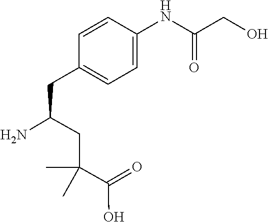 [SMILES: CC(C)(C[C@@H](N)Cc1ccc(NC(=O)CO)cc1)C(=O)O]

To a solution of TUP-6b (0.34 g, 1.0 mmol) in DCM (5.0 mL) was added 2,6-lutidine (21 mg, 2.0 mmol), DMAP (12 mg, 0.10 mmol), and benzyloxyacetyl chloride (TUP-7e) (0.22 g, 1.2 mmol). The reaction mixture was stirred at room temperature for 3 hours, and monitored by LCMS. The resulting mixture was diluted with ethyl acetate (50 mL), washed with water and brine, dried over anhydrous sodium sulfate, and concentrated in vacuo. The residue was purified by reversed phase flash chromatography (0-100% acetonitrile in aq. TFA (0.3%)) to give compound TUP-8be′ (0.22 g, 45% yield) as a white solid. ESI m/z 385 (M −Boc+H)⁺.

To a solution of compound TUP-8be′ (0.10 g, 0.21 mmol) in methanol (5 mL) was added 10% palladium on carbon (20 mg) under nitrogen. The mixture was degassed and purged with hydrogen 3 times. The reaction was then stirred at room temperature under a hydrogen balloon for 3 hours, and monitored by LCMS. The reaction mixture was diluted with methanol and filtered through Celite. The filtrate was concentrated in vacuo to give crude compound TUP-8be (80 mg, >100% crude yield) as a white solid. ESI m/z 395 (M+H)⁺.

To a solution of crude TUP-8be (39 mg, 0.10 mmol) in DCM (5 mL) was added TFA (1.0 mL). The mixture was stirred at room temperature for 2 hours until Boc was totally removed in vacuo, according to LCMS. The volatiles were removed in vacuo to give crude compound TUPk (30 mg, >100% crude yield) as a white solid. ESI m/z 295 (M+H)⁺.

### (4S)-4-Amino-5-{4-[(2-{[(9H-fluoren-9-ylmethoxy)carbonyl]amino}ethyl)amino]phenyl}-2,2-dimethylpentanoic Acid (TUPl)

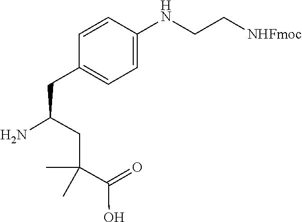

To a solution of TUP-6b (0.20 g, 0.60 mmol) in DCE (25 mL) was subsequently added Fmoc-aminoacetaldehyde (0.17 g, 0.60 mmol) and sodium triacetoxyborohydride (0.13 g, 0.60 mmol). The reaction mixture was stirred at room temperature for an hour, and monitored by LCMS. The resulting mixture was quenched with sat. aq. sodium bicarbonate at 0° C. The organic layer was washed with sat. aq. sodium bicarbonate and brine, dried over anhydrous magnesium sulfate, and filtered. The filtrate was concentrated in vacuo and the crude product was purified by silica gel column chromatography (0-50% ethyl acetate in petroleum ether) to give TUP-8bf (70 mg, 19% yield) as a white solid. ESI m/z: 602 (M+H)⁺.

To a solution of TUP-8bf (70 mg, 0.12 mmol) in DCM (5 mL) was added TFA (1.0 mL), and the mixture was stirred at room temperature for 2 hours until Boc was totally removed in vacuo according to LCMS. The volatiles were removed in vacuo. The residue was purified by prep-HPLC (0-100% acetonitrile in aq. ammonium bicarbonate (10 mM)) to give compound TUPl (56 mg, 96% yield) as a white solid. ESI m/z 502 (M+H)⁺.

General Procedure I

Amidation With MEP: Synthesis of Intermediate 2

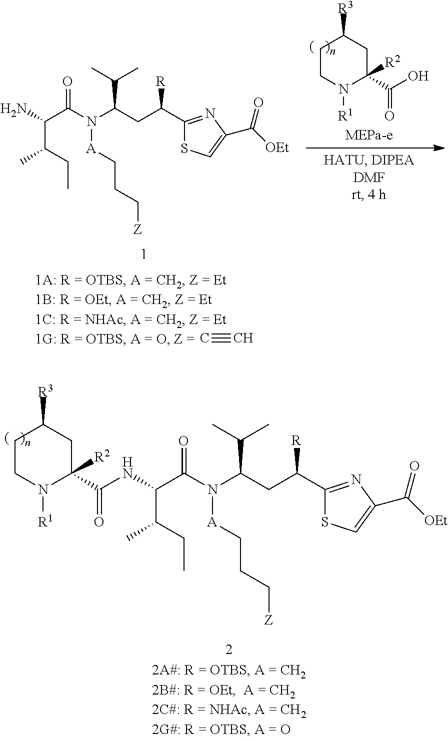

To a solution of intermediate 1A-C,G (1.0 equiv) in DMF (20 mM) was subsequently added DIPEA (2.0 equiv), HATU (1.5 equiv) and acid MEPa-e (1.2 equiv) at 0° C. The reaction mixture was stirred at room temperature for an hour until the starting material was consumed, according to LCMS. The resulting mixture was quenched with water, and extracted with ethyl acetate (x 3). The combined organic solution was washed with brine, dried over anhydrous sodium sulfate, and concentrated in vacuo to give crude amide 2. Crude amide 2 was used in the next step without further purification.

General Procedure II

TBS-Deprotection: From 2A # to 2D # and From 2G # to 2H #

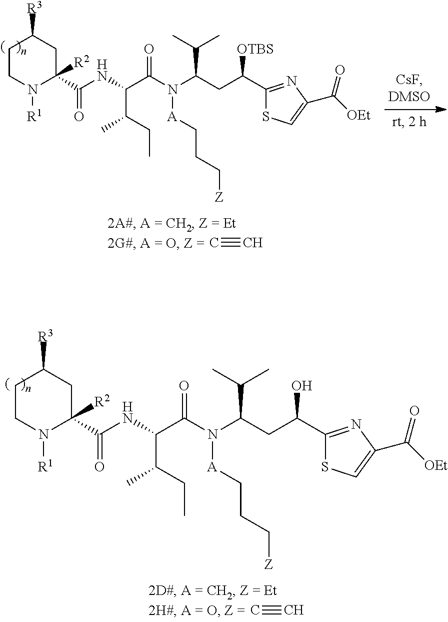

To a solution of TBS protected compounds 2A # or 2G # (1.0 equiv) in DMSO (0.15-0.20 mM) was added cesium fluoride (2.0 equiv). The mixture was stirred at room temperature for 2 hours, and monitored by LCMS. The mixture was filtered and the filtrate was concentrated in vacuo. The residue was purified by reversed phase flash chromatography (0-70% acetonitrile in water) to give alcohols 3D # or 2H # as oils.

General Procedure III

Synthesis of Carbamates 2E #

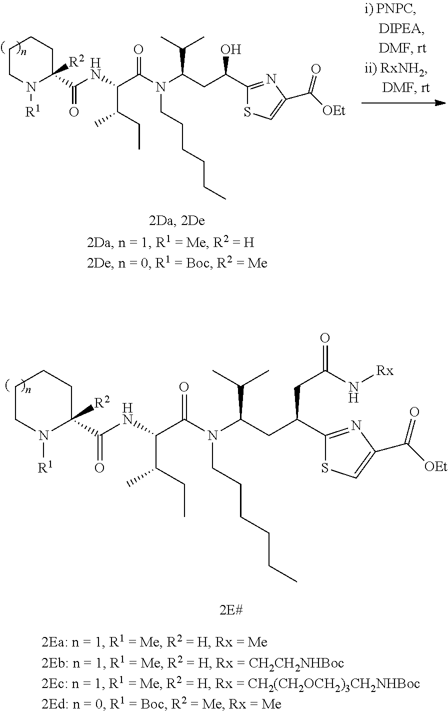

To a solution of compound 2 Da or 2De (1.0 equiv) in DMF (25 mM) was added DIPEA (3.0 equiv) and 4-nitrobenzoic anhydride (5.0 equiv). The mixture was stirred at room temperature for 16 hours, and monitored by LCMS. The reaction solution was diluted with water and extracted with ethyl acetate (x 3). The combined organic solution was dried over anhydrous sodium sulfate and concentrated in vacuo. The residue was dissolved in DMF (50 mM). To the solution was added amine (RxNH₂) (2.0 equiv) and DIPEA (2.0 equiv). The mixture was stirred at room temperature for an hour, and monitored by LCMS. The resulting mixture was purified directly by reversed phase flash chromatography (5-95% acetonitrile in water) to give compound 2E # (60-71% yield in 2 steps from 2D #) as a light yellow solid.

General Procedure IV

Hydrolysis to Obtain Acids 3

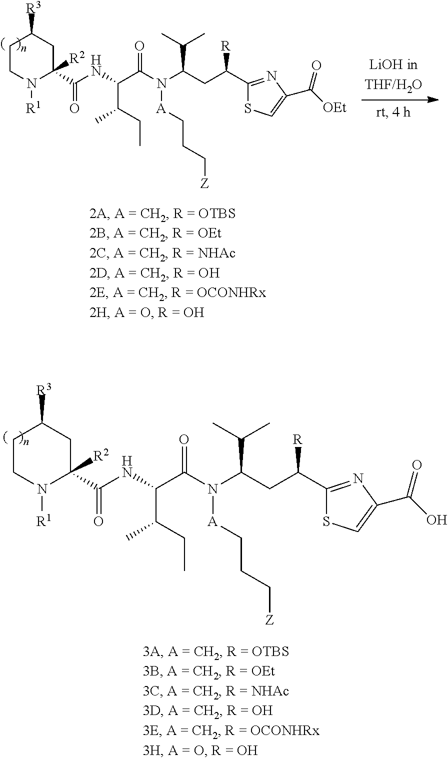

To a solution of ethyl ester 2A-E,H (1.0 equiv) in THE (0.1 M) was added aq. lithium hydroxide (0.5 M, 6.0 equiv). The mixture was stirred at room temperature for 4 hours until the hydrolysis was completed, according to LCMS. The reaction mixture was then acidified with acetic acid to pH 3 and concentrated to ⅓ volume. The residual aqueous solution was extracted with ethyl acetate (x3) and the combined organic layer was washed with brine, dried over anhydrous sodium sulfate, and concentrated in vacuo to give the corresponding acid 3A-E,H. Acid 3A-E,H was used in the next step without further purification.

General Procedure V

Acetylation of 3F and 3I

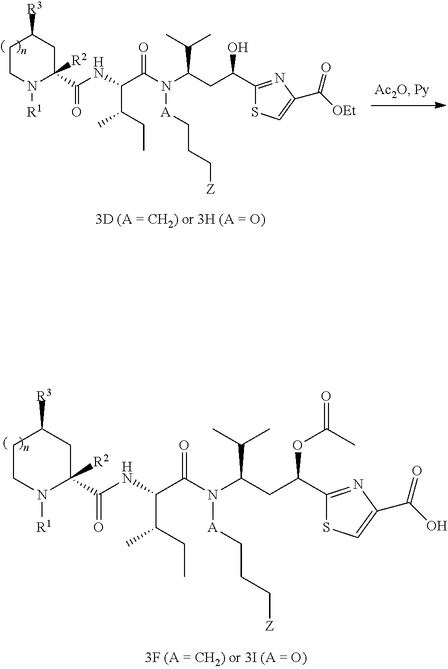

To a solution of compound 3D or 3H (1.0 equiv) in pyridine (50-60 mM) was added acetic anhydride (2.0 equiv) and DMAP (0.02 equiv). The reaction mixture was stirred at room temperature for 4-16 hours, and monitored by LCMS. The resulting mixture was concentrated in vacuo, and the residue was purified by reversed phase flash chromatography (0-25% acetonitrile in aq. ammonium bicarbonate (0.08%)) to give compound 3F or 31 as a white solid.

General Procedure VI

Synthesis of Tubulysin Payloads or Protected Tubulysin Payloads

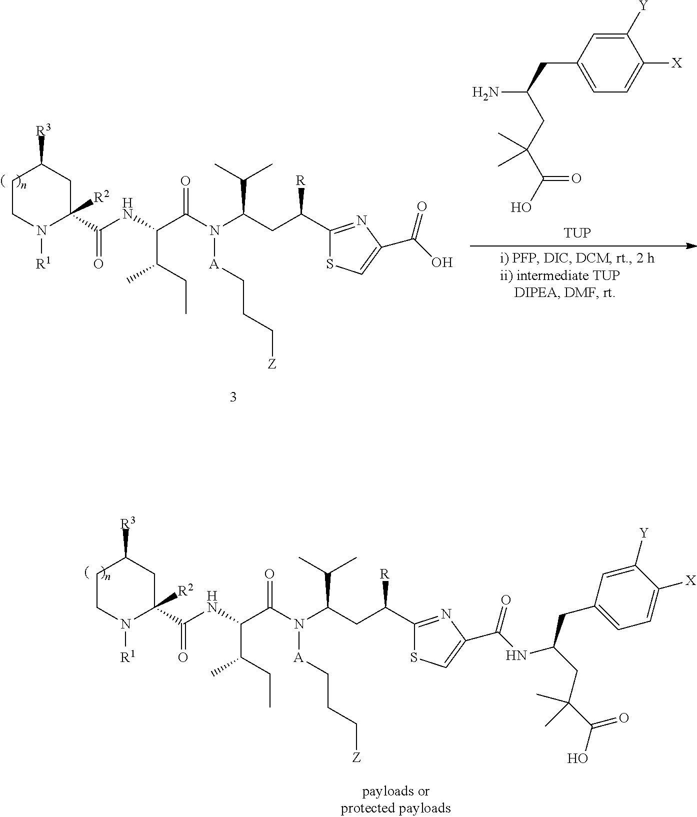

To a solution of acid 3 (1.0 equiv) in DCM (30 mM) was added pentafluorophenol (PFP) (2.5 equiv) and N,N′-diisopropylcarbodiimide (DIC) (2.5 equiv). The reaction mixture was stirred at room temperature for 2 hours, and monitored by LCMS. The resulting mixture was concentrated in vacuo to give pentafluorophenol ester, which was dissolved in DCM (50 mM). To the solution was added intermediate TUP (1.5 equiv) and DIPEA (4.0 equiv). The reaction mixture was stirred at room temperature for 4 hours, and monitored by LCMS. The resulting mixture was purified directly by prep-HPLC to give the corresponding amide (7-57% yield, protected tubulysin payload or tubulysin payload directly) as a white solid.

General Procedure VII

Synthesis of N-acylsulfonamides

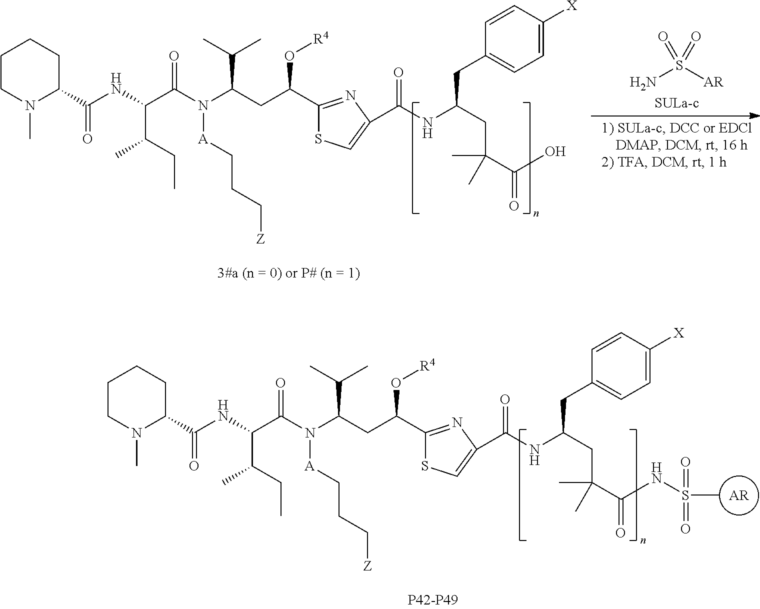

To a stirred mixture of sulfonamide SULa-c (1.0 equiv), acid 3 #a or P # (1.0 equiv), and DMAP (1.5 equiv) in DCM (25 mM) was added DCC (1.5 equiv) or EDCI (1.2 equiv) at room temperature. The resulting solution was stirred at room temperature overnight, and monitored by LCMS. The reaction mixture was concentrated and the residue was purified by reversed phase flash chromatography (0-100% acetonitrile in water) to give crude N-acylsulfonamides containing DCU. The crude was repurified by prep-HPLC (0-100% acetonitrile in aq. ammonium bicarbonate (10 mM)) to give pure Boc-payload as a white solid, which was dissolved in DCM (2.5 mM). To the solution was added TFA (VTFA/VDCM=1:1), and the reaction mixture was stirred at room temperature for an hour until Boc was totally removed, according to LCMS. The resulting mixture was concentrated in vacuo and the residue was purified by prep-HPLC (5-100% acetonitrile in aq. ammonium bicarbonate (10 mM)) to give payload P42-49 as a white solid.

General Procedure VIII

Synthesis of vc-Tub and vcPAB-Tub (L1-3a-d)

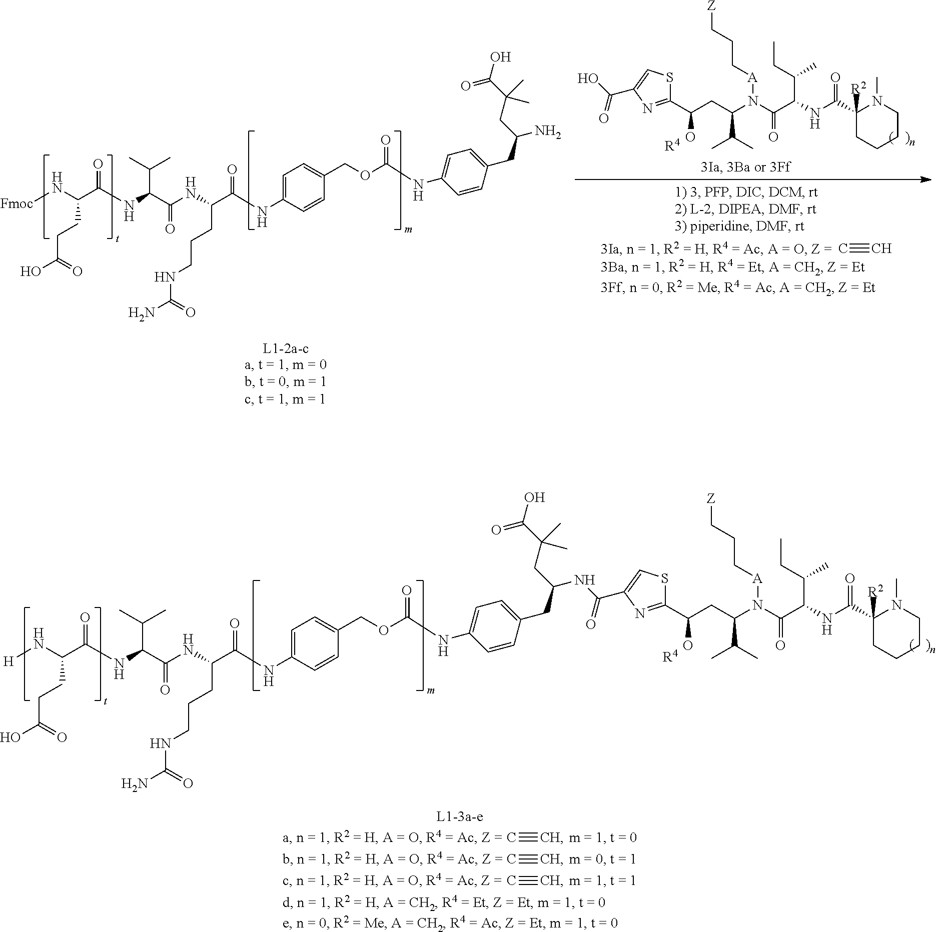

To a solution of acid 3 (1.0 equiv) in DCM (30 mM) were added pentafluorophenol (PFP) (2.5 equiv) and N,N′-diisopropylcarbodiimide (DIC) (2.5 equiv). The reaction mixture was stirred at room temperature for 2 hours, and monitored by LCMS. The resulting mixture was concentrated in vacuo to give the corresponding pentafluorophenol ester, which was added into a mixture of compound L1-2 (1.0 equiv) and DIPEA (3.0 equiv) in DMF (15 mM). The reaction mixture was stirred at room temperature overnight, and monitored by LCMS. The resulting mixture was purified directly by reversed phase flash chromatography (0-100% acetonitrile in water) to give compound Fmoc-L1-3 as a white solid, which was dissolved in DMF (40 mM). To the solution was added piperidine (3.0 equiv), and the mixture was stirred at room temperature for 2 hours until Fmoc was totally removed, according to LCMS. The resulting mixture was purified directly by reversed phase flash chromatography (0-100% acetonitrile in aq. TFA (0.01%)) to give compound L1-3 (25-67% yield in 3 steps from acid 3).

General Procedure IX

Amidation From Amines With OSu Esters

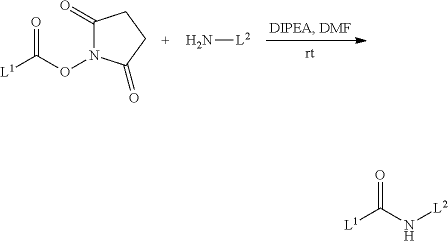

To a solution of amine (L²-NH₂) (1.0 equiv) in DMF (10 mM) was added OSu ester (L¹-COOSu) (1.2-1.3 equiv) and DIPEA (2.5-3.0 equiv). The reaction solution was stirred at room temperature for 2 hours, and monitored by LCMS. The resulting solution was purified directly by reversed phase flash chromatography (0-100% acetonitrile in aq. ammonium bicarbonate (10 mM)) to give amide (Lt-CONH-L²) as a white solid.

General Procedure X

Synthesis of Carbamates From Amines with vcPAB-PNP Esters

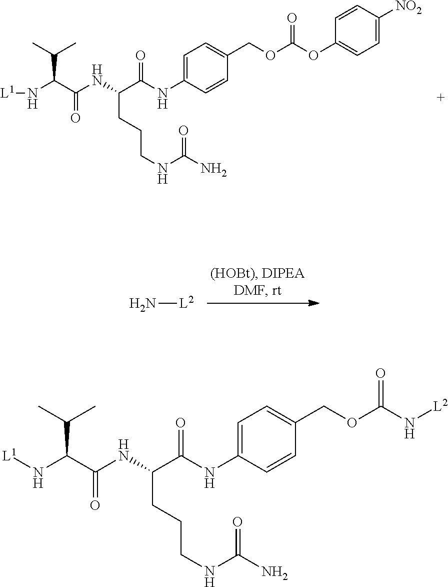

To a solution of amine (L²-NH₂) (1.0 equiv) in DMF (16 mM) was added Lt-vcPAB-PNP (1.0 equiv), HOBt (1.0 equiv or without HOBt), and DIPEA (3.0 equiv). The mixture was stirred at room temperature for 1-4 hours, and monitored by LCMS. The reaction mixture was purified directly by reversed phase flash chromatography (0-100% acetonitrile in aq. ammonium bicarbonate (10 mM)) to give the desired carbamate as a white solid.

TABLE 1-1
Compound List of Tubulysins

| | | | | | | HPLC | HPLC |
|---|---|---|---|---|---|---|---|
| | | | | | | purity | RT |
| # | Structures | cLogP | MF | MW | Mass m/z | (%) | (min) |
| P1 | 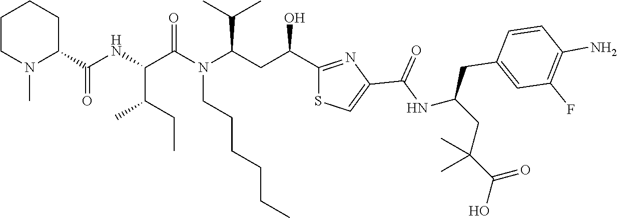 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] | 4.04 | C₄₂H₆₇FN₆O₆S | 803.1 | 402 (M/2 + H) | >99 | 6.93 (A) |
| P3 | 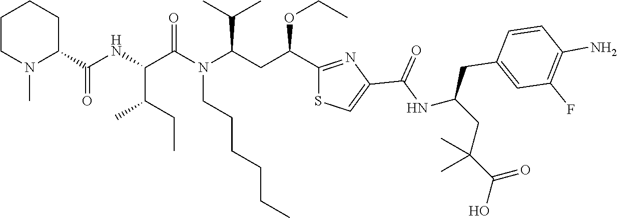 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] | 5.04 | C₄₄H₇₁FN₆O₆S | 831.1 | 831.5 (M + H) | >99 | 9.18 (B) |
| P5 | 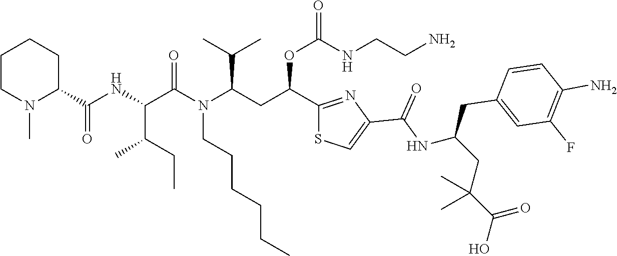 [SMILES: CCCCCCN(C(=O)[C@@H](NC(=O)[C@H]1CCCCN1C)[C@@H](C)CC)[C@H](C[C@@H](OC(=O)NCCN)c1nc(C(=O)N[C@@H](Cc2ccc(N)c(F)c2)CC(C)(C)C(=O)O)cs1)C(C)C] | 3.87 | C₄₅H₇₃FN₈O₇S | 889.2 | 445 (M/2 + H) | >99 | 8.09 (B) |
| P6 | 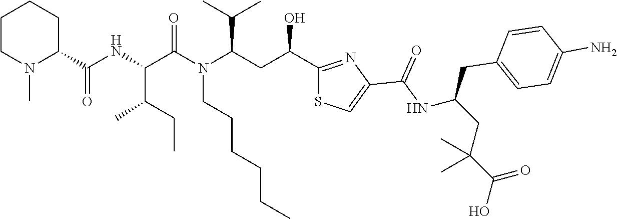 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)cc2)CC(C)(C)C(=O)O)cs1)C(C)C] | 3.90 | C₄₂H₆₈FN₆O₆S | 785.1 | 393 (M/2 + H) | 99 | 6.13 (A) |
| P7 | 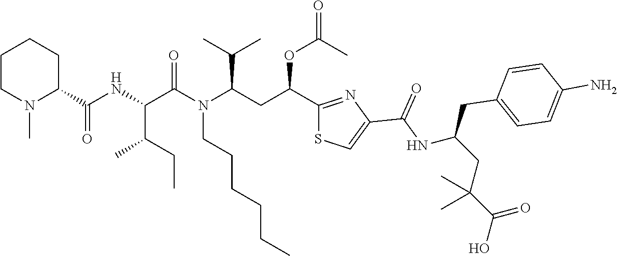 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] | 4.34 | C₄₄H₇₀N₆O₇S | 827.1 | 828 (M + H) | 99 | 5.59 (A) |
| P8 | 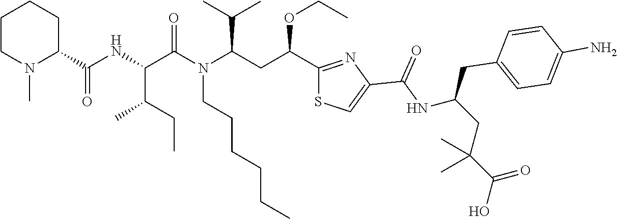 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)cc2)CC(C)(C)C(=O)O)cs1)C(C)C] | 4.90 | C₄₄H₇₂N₆O₆S | 813.2 | 813.5 (M + H) | 99 | 8.93 (B) |
| P9 | 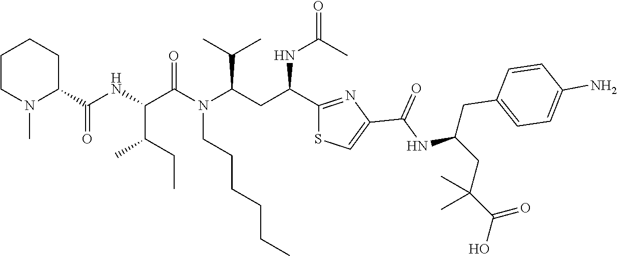 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(N)cc2)CC(C)(C)C(=O)O)cs1)C(C)C] | 3.62 | C₄₄H₇₁N₇O₆S | 826.2 | 826 (M + H) | >99 | 7.98 (B) |
| P10 | 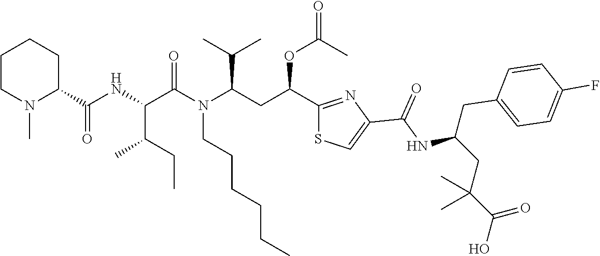 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)O)cs1)C(C)C] | 5.31 | C₄₄H₆₈FN₅O₇S | 830.1 | 830.5 (M + H) | >99 | 9.63 (B) |
| P11 | 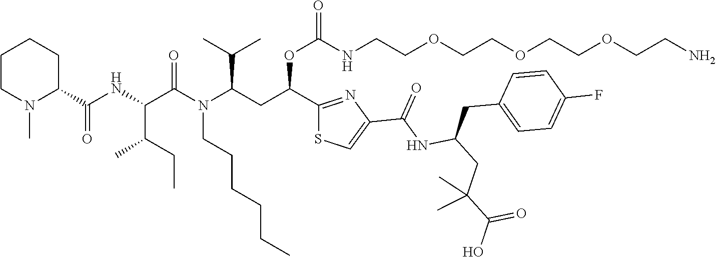 [SMILES: CCCCCCN(C(=O)[C@@H](NC(=O)[C@H]1CCCCN1C)[C@@H](C)CC)[C@H](C[C@@H](OC(=O)NCCOCCOCCOCCN)c1nc(C(=O)N[C@@H](Cc2ccc(F)cc2)CC(C)(C)C(=O)O)cs1)C(C)C] | 4.57 | C₅₁H₈₄FN₇O₁₀S | 1006 | 504 (M/2 + H) | 99 | 6.62 (A) |

TABLE 1-2
Cytotoxicity of Tubulysin Payloads Modified on the R group

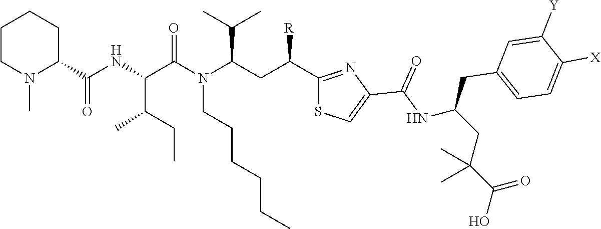

| | | | | HCT- | HCT-15 with |
|---|---|---|---|---|---|
| | Structures | | | 15 IC₅₀ | verapamil |
| # | R | X | Y | (nM) | IC₅₀ (nM) |
| P1 | OH | NH₂ | F | 3.00 | 0.26 |
| P3 | OEt | NH₂ | F | 0.07 | 0.01 |
| P5 | OCONHCH₂CH₂NH₂ | NH₂ | F | 38.6 | 4.44 |
| P6 | OH | NH₂ | H | 16.3 | 0.78 |
| P7 | OAc | NH₂ | H | 0.02 | 0.02 |
| P8 | OEt | NH₂ | H | 0.24 | 0.06 |
| P9 | NHAc | NH₂ | H | 2.07 | 0.30 |
| P10 | OAc | F | H | 0.24 | 0.09 |
| P11 | OCONH(CH₂CH₂O)₃CH₂CH₂NH₂ | F | H | 157 | |

Synthesis of Intermediates 2Aa, 2B, 2C, and 2 Da

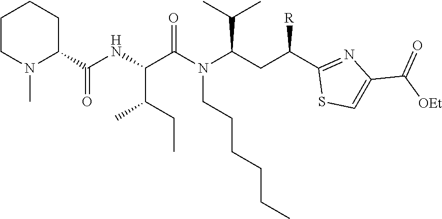

### Ethyl 2-[(1R,3R)-1-[(tert-butyldimethylsilyl)oxy]-3-[(2S,3S)—N-hexyl-3-methyl-2-{[(2R)-1-methylpiperidin-2-yl]formamido}pentanamido]-4-methylpentyl]-1,3-thiazole-4-carboxylate (2Aa)

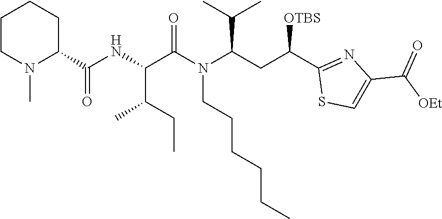 [SMILES: CCCCCCN(C(=O)[C@@H](NC(=O)[C@H]1CCCCN1C)[C@@H](C)CC)[C@H](C[C@@H](O[Si](C)(C)C(C)(C)C)c1nc(C(=O)OCC)cs1)C(C)C]

Following General Procedure I starting from intermediate 1A (54 mg, 92 μmol) with acid MEPa, crude compound 2Aa (60 mg, crude) was obtained as a white solid. ESI m/z: 710 (M+H)⁺.

### Ethyl 2-[(1R,3R)-1-ethoxy-3-[(2S,3S)—N-hexyl-3-methyl-2-{[(2R)-1-methylpiperidin-2-yl]formamido}pentanamido]-4-methylpentyl]-1,3-thiazole-4-carboxylate (2B)

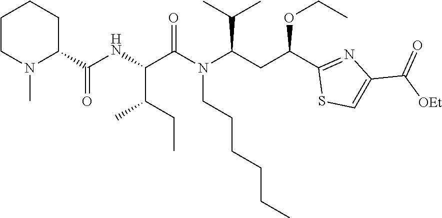 [SMILES: CCCCCCN(C(=O)[C@@H](NC(=O)[C@H]1CCCCN1C)[C@@H](C)CC)[C@H](C[C@@H](OCC)c1nc(C(=O)OCC)cs1)C(C)C]

Following General Procedure I starting from intermediate 1B (50 mg, 0.10 mmol) with acid MEPa, compound 2B (31 mg, 50% yield) was obtained as a white solid after purification by prep-HPLC (Method B). ESI m/z: 623 (M+H)⁺.

### Ethyl 2-[(1R,3R)-1-acetamido-3-[(2S,3S)—N-hexyl-3-methyl-2-{[(2R)-1-methylpiperidin-2-yl]formamido}pentanamido]-4-methylpentyl]-1,3-thiazole-4-carboxylate (2C)

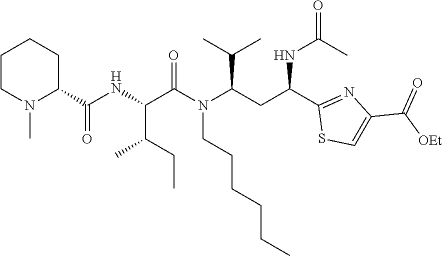 [SMILES: CCCCCCN(C(=O)[C@@H](NC(=O)[C@H]1CCCCN1C)[C@@H](C)CC)[C@H](C[C@@H](NC(C)=O)c1nc(C(=O)OCC)cs1)C(C)C]

Following General Procedure I starting from intermediate 1C (50 mg, 98 μmol) with acid MEPa, compound 2C (50 mg, 80% crude yield) was obtained as a yellow oil. ESI m/z: 636 (M+H)⁺.

### Ethyl 2-[(1R,3R)-3-[(2S,3S)—N-hexyl-3-methyl-2-{[(2R)-1-methylpiperidin-2-yl]formamido}pentanamido]-1-hydroxy-4-methylpentyl]-1,3-thiazole-4-carboxylate (2 Da)

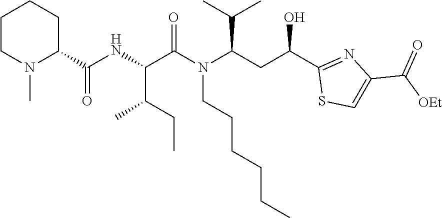 [SMILES: CCCCCCN(C(=O)[C@@H](NC(=O)[C@H]1CCCCN1C)[C@@H](C)CC)[C@H](C[C@@H](O)c1nc(C(=O)OCC)cs1)C(C)C]

Following General Procedure II starting from crude compound 2Aa (0.50 g) in DMSO (6 mL), compound 2 Da (0.32 g, 75% yield in 2 steps) was obtained as a light yellow oil. ESI m/z: 595 (M+H)⁺.

Synthesis of Carbamates 2Ea, 2Eb, and 2Ec

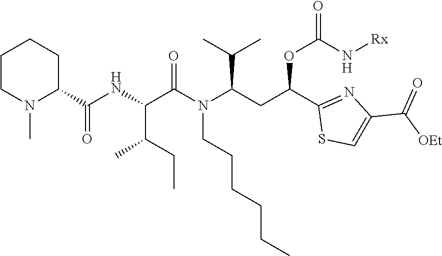

### Ethyl 2-[(1R,3R)-3-[(2S,3S)—N-hexyl-3-methyl-2-{[(2R)-1-methylpiperidin-2-yl]formamido}pentanamido]-4-methyl-1-[(methylcarbamoyl)oxy]pentyl]-1,3-thiazole-4-carboxylate (2Ea)

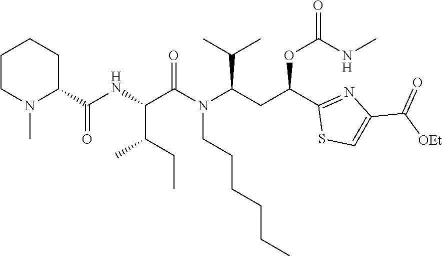 [SMILES: CCCCCCN(C(=O)[C@@H](NC(=O)[C@H]1CCCCN1C)[C@@H](C)CC)[C@H](C[C@@H](OC(=O)NC)c1nc(C(=O)OCC)cs1)C(C)C]

Following General Procedure III using methylamine, carbamate 2Ea (30 mg, 71% yield in 2 steps from 2 Da) was obtained as a light yellow solid. ESI m/z: 652 (M+H)⁺.

### Ethyl 2-[(1R,3R)-1-{[(2-{[(tert-butoxy)carbonyl]amino}ethyl)carbamoyl]oxy}-3-[(2S,3S)—N-hexyl-3-methyl-2-{[(2R)-1-methylpiperidin-2-yl]formamido}pentanamido]-4-methylpentyl]-1,3-thiazole-4-carboxylate (2Eb)

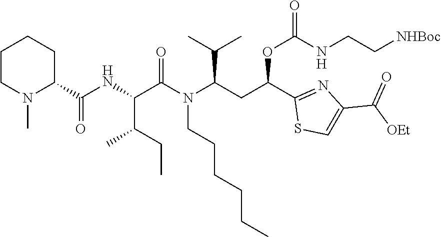 [SMILES: CCCCCCN(C(=O)[C@@H](NC(=O)[C@H]1CCCCN1C)[C@@H](C)CC)[C@H](C[C@@H](OC(=O)NCCNC(=O)OC(C)(C)C)c1nc(C(=O)OCC)cs1)C(C)C]

Following General Procedure III using N-Boc-ethylenediamine, carbamate 2Eb (78 mg, 60% yield in 2 steps from 2 Da) was obtained as a light yellow solid (contaminated with a trace amount of 2 Da according to LCMS). ESI m/z: 781 (M+H)⁺.

### Ethyl 2-[(1R,3R)-1-{[(2-{2-[2-(2-azidoethoxy)ethoxy]ethoxy}ethyl)carbamoyl]oxy}-3-[(2S,3S)—N-hexyl-3-methyl-2-{[(2R)-1-methylpiperidin-2-yl]formamido}pentanamido]-4-methylpentyl]-1,3-thiazole-4-carboxylate (2Ec)

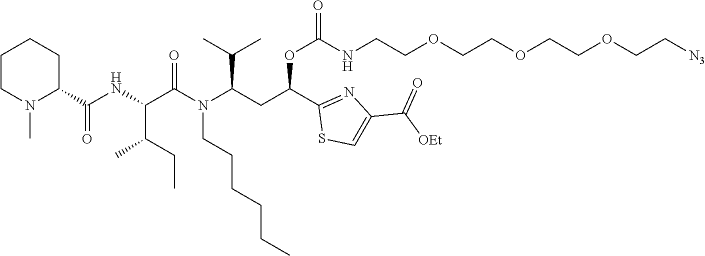 [SMILES: CCCCCCN(C(=O)[C@@H](NC(=O)[C@H]1CCCCN1C)[C@@H](C)CC)[C@H](C[C@@H](OC(=O)NCCOCCOCCOCCN=[N+]=[N-])c1nc(C(=O)OCC)cs1)C(C)C]

Following General Procedure III using 11-azido-3,6,9-trioxaundecan-1-amine, carbamate 2Ec (0.22 g, 64% yield in 2 steps from 2 Da) was obtained as a light yellow oil. ESI m/z: 839 (M+H)⁺. ¹H NMR (400 MHz, DMSO_{d6}) δ 8.40 (s, 1H), 7.62 (d, J=7.2 Hz, 1H), 7.55 (t, J=4.8 Hz, 1H), 5.55 (d, J=10.0 Hz, 1H), 4.48 (t, J=7.6 Hz, 1H), 4.30 (q, J=5.6 Hz, 2H), 3.61-3.58 (m, 2H), 3.55-3.50 (m, 8H), 3.40-3.37 (m, 4H), 3.32-3.30 (m, 1H), 3.14-3.07 (m, 2H), 2.99-2.94 (m, 1H), 2.83-3.80 (m, 1H), 2.48-2.45 (m, 1H), 2.15-2.09 (m, 1H), 2.06 (s, 3H), 1.95-1.77 (m, 4H), 1.62-1.41 (m, 6H), 1.36-1.23 (m, 12H), 1.13-1.05 (m, 2H), 0.92 (d, J=7.5 Hz, 3H), 0.89-0.81 (m, 9H), 0.69 (br s, 3H) ppm.

Synthesis of Intermediate 3Aa, 3Ba, 3C, 3 Da, 3Ea, 3Eb, 3Ec, and 3Fa

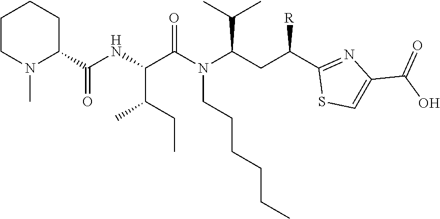

### 2-[(1R,3R)-1-[(tert-Butyldimethylsilyl)oxy]-3-[(2S,3S)—N-hexyl-3-methyl-2-{[(2R)-1-methylpiperidin-2-yl]formamido}pentanamido]-4-methylpentyl]-1,3-thiazole-4-carboxylic acid (3Aa)

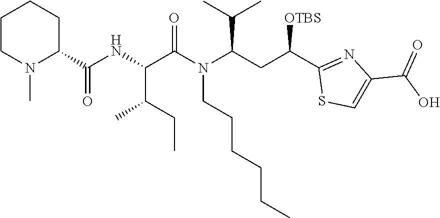 [SMILES: CCCCCCN(C(=O)[C@@H](NC(=O)[C@H]1CCCCN1C)[C@@H](C)CC)[C@H](C[C@@H](O[Si](C)(C)C(C)(C)C)c1nc(C(=O)O)cs1)C(C)C]

Following General Procedure IV from 2Aa (0.27 g, crude), acid 3Aa (0.18 g, 70% yield in 2 steps from intermediate 1A) was obtained as a yellow solid after purification by prep-HPLC (Method A). ESI m/z: 681 (M+H)⁺.

### 2-[(1R,3R)-1-Ethoxy-3-[(2S,3S)—N-hexyl-3-methyl-2-{[(2R)-1-methylpiperidin-2-yl]formamido}pentanamido]-4-methylpentyl]-1,3-thiazole-4-carboxylic acid (3Ba)

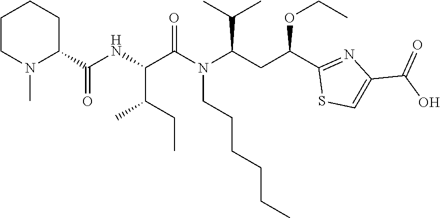 [SMILES: CCCCCCN(C(=O)[C@@H](NC(=O)[C@H]1CCCCN1C)[C@@H](C)CC)[C@H](C[C@@H](OCC)c1nc(C(=O)O)cs1)C(C)C]

Following General Procedure IV from 2Ba (62 mg, 0.10 mmol), acid 3Ba (46 mg, 80% yield) was obtained as a white solid after purification by reversed phase flash chromatography (5-100% acetonitrile in aq. TFA (0.03%)). ESI m/z: 595 (M+H)⁺.

### 2-[(1R,3R)-1-Acetamido-3-[(2S,3S)—N-hexyl-3-methyl-2-{[(2R)-1-methylpiperidin-2-yl]formamido}pentanamido]-4-methylpentyl]-1,3-thiazole-4-carboxylic Acid (3C)

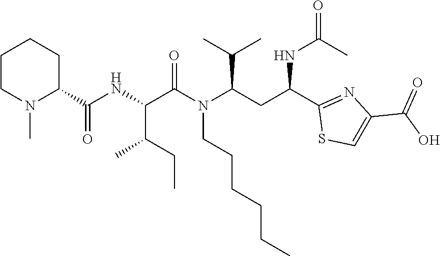 [SMILES: CCCCCCN(C(=O)[C@@H](NC(=O)[C@H]1CCCCN1C)[C@@H](C)CC)[C@H](C[C@@H](NC(C)=O)c1nc(C(=O)O)cs1)C(C)C]

Following General Procedure IV from 2C (50 mg, 79 mmol), acid 3C (40 mg, 84% yield) was obtained as a white solid after purification by prep-HPLC (Method A). ESI m/z: 608 (M+H)⁺.

### 2-[(1R,3R)-3-[(2S,3S)—N-Hexyl-3-methyl-2-{[(2R)-1-methylpiperidin-2-yl]formamido}pentanamido]-1-hydroxy-4-methylpentyl]-1,3-thiazole-4-carboxylic Acid (3 Da)

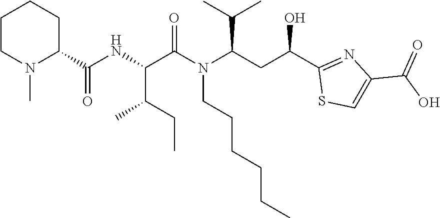 [SMILES: CCCCCCN(C(=O)[C@@H](NC(=O)[C@H]1CCCCN1C)[C@@H](C)CC)[C@H](C[C@@H](O)c1nc(C(=O)O)cs1)C(C)C]

Following General Procedure IV from 2 Da (0.15 g, 0.24 mmol), crude acid 3 Da (0.14 g, 94% yield) was obtained as an off-white solid, and used in the next step without further purification. ESI m/z: 567 (M+H)⁺.

### 2-[(1R,3R)-3-[(2S,3S)—N-Hexyl-3-methyl-2-{[(2R)-1-methylpiperidin-2-yl]formamido}pentanamido]-4-methyl-1-[(methylcarbamoyl)oxy]pentyl]-1,3-thiazole-4-carboxylic Acid (3Ea)

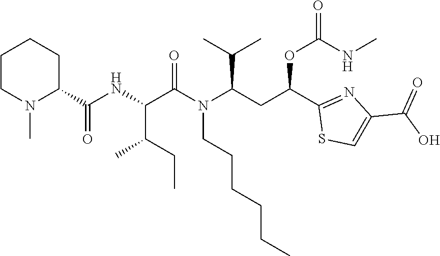 [SMILES: CCCCCCN(C(=O)[C@@H](NC(=O)[C@H]1CCCCN1C)[C@@H](C)CC)[C@H](C[C@@H](OC(=O)NC)c1nc(C(=O)O)cs1)C(C)C]

Following General Procedure IV from 2Ea, acid 3Ea (0.10 g, 85% yield) was obtained as a white solid, and used in the next step without further purification. ESI m/z: 624 (M+H)⁺.

### 2-[(1R,3R)-1-{[(2-{[(tert-Butoxy)carbonyl]amino}ethyl)carbamoyl]oxy}-3-[(2S,3S)—N-hexyl-3-methyl-2-{[(2R)-1-methylpiperidin-2-yl]formamido}pentanamido]-4-methylpentyl]-1,3-thiazole-4-carboxylic Acid (3Eb)

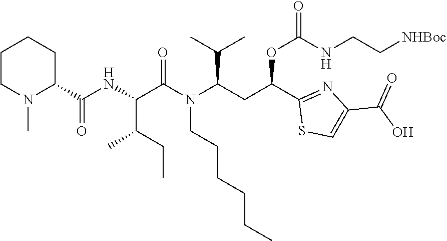 [SMILES: CCCCCCN(C(=O)[C@@H](NC(=O)[C@H]1CCCCN1C)[C@@H](C)CC)[C@H](C[C@@H](OC(=O)NCCNC(=O)OC(C)(C)C)c1nc(C(=O)O)cs1)C(C)C]

Following General Procedure IV from 2Eb, acid 3Eb (52 mg, 70% yield) was obtained as a white solid after purification by prep-HPLC (Method B). ESI m/z: 753 (M+H)⁺. ¹H NMR (400 MHz, DMSO_{d6}) δ 7.74 (s, 1H), 7.60 (s, 1H), 7.42 (s, 1H), 6.80 (s, 1H), 5.50 (d, J=8.4 Hz, 1H), 4.48 (t, J=9.2 Hz, 1H), 3.65-3.57 (m, 1H), 2.97 (s, 5H), 2.81 (d, J=11.6 Hz, 1H), 2.49-2.45 (m, 1H), 2.20-2.11 (m, 2H), 2.08 (s, 3H), 1.94-1.88 (m, 3H), 1.82-1.75 (m, 1H), 1.70-1.44 (m, 6H), 1.37 (s, 10H), 1.29 (s, 6H), 1.22-1.04 (m, 2H), 0.93 (d, J=6.4 Hz, 3H), 0.88-0.80 (m, 10H), 0.72 (br s, 3H) ppm.

### 2-[(1R,3R)-1-{[(2-{2-[2-(2-Azidoethoxy)ethoxy]ethoxy}ethyl)carbamoyl]oxy}-3-[(2S,3S)—N-hexyl-3-methyl-2-{[(2R)-1-methylpiperidin-2-yl]formamido}pentanamido]-4-methylpentyl]-1,3-thiazole-4-carboxylic Acid (3Ec)

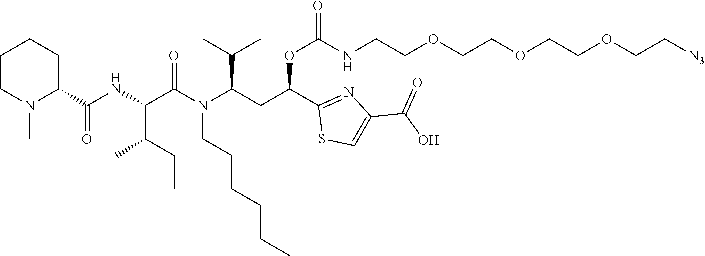 [SMILES: CCCCCCN(C(=O)[C@@H](NC(=O)[C@H]1CCCCN1C)[C@@H](C)CC)[C@H](C[C@@H](OC(=O)NCCOCCOCCOCCN=[N+]=[N-])c1nc(C(=O)O)cs1)C(C)C]

Following General Procedure IV from 2Ec, acid 3Ec (0.20 g, 94% yield) was obtained as a colorless viscous oil, and used in the next step without further purification. ESI m/z: 811.5 (M+H)⁺.

### 2-[(1R,3R)-1-(Acetyloxy)-3-[(2S,3S)—N-hexyl-3-methyl-2-{[(2R)-1-methylpiperidin-2-yl]formamido}pentanamido]-4-methylpentyl]-1,3-thiazole-4-carboxylic Acid (3Fa)

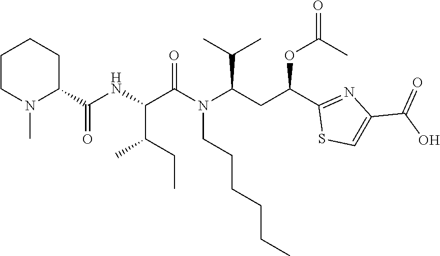 [SMILES: CCCCCCN(C(=O)[C@@H](NC(=O)[C@H]1CCCCN1C)[C@@H](C)CC)[C@H](C[C@@H](OC(C)=O)c1nc(C(=O)O)cs1)C(C)C]

Following General Procedure V from compound 3 Da (0.13 g, 0.22 mmol), acid 3Fa (0.12 g, 90% yield) was obtained as a white solid after purification by reversed phase flash chromatography (0-25% acetonitrile in aq. ammonium bicarbonate (0.08%)). ESI m/z: 609 (M+H)⁺.

Synthesis of Tubulysin Payloads in Table 1

### P1: (4S)-5-(4-amino-3-fluorophenyl)-4-({2-[(1R,3R)-3-[(2S,3S)—N-hexyl-3-methyl-2-{[(2R)-1-methylpiperidin-2-yl]formamido}pentanamido]-1-hydroxy-4-methylpentyl]-1,3-thiazol-4-yl}formamido)-2,2-dimethylpentanoic Acid (P1)

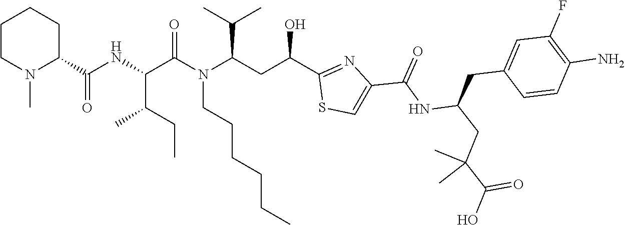 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C]

To a solution of P2 (see P2) (20 mg, 23 μmol) in aq. THF (80 vol %, 2.0 mL) was added lithium hydroxide (11 mg, 0.23 mmol), and the mixture was stirred at room temperature overnight, and monitored by LCMS. The reaction mixture was then acidified by aq. HCl (1 M) to pH 3, and extracted with ethyl acetate. The combined organic solution was dried over sodium sulfate and concentrated in vacuo. The residue was purified by prep-HPLC (0-100% acetonitrile in aq. ammonium bicarbonate (10 mM)) to give payload P1 (17 mg, 90% yield) as a white solid. ESI m/z: 402 (M/2+H)⁺, 804.5 (M+H)⁺. ¹H NMR (400 MHz, methanol_{d4}) δ 8.05 (s, 1H), 6.88-6.74 (m, 3H), 4.69-4.61 (m, 2H), 4.33-4.31 (m, 1H), 3.82-3.76 (m, 1H), 3.02-2.95 (m, 1H), 2.81-2.70 (m, 3H), 2.30-2.29 (m, 1H), 2.20-2.14 (m, 5H), 2.00-1.94 (m, 3H), 1.76-1.55 (m, 9H), 1.40-1.21 (m, 9H), 1.19 (s, 3H), 1.16-1.13 (m, 4H), 1.05-0.90 (m, 15H) ppm.

### P3: (4S)-5-(4-amino-3-fluorophenyl)-4-({2-[(1R,3R)-1-ethoxy-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic Acid (P3)

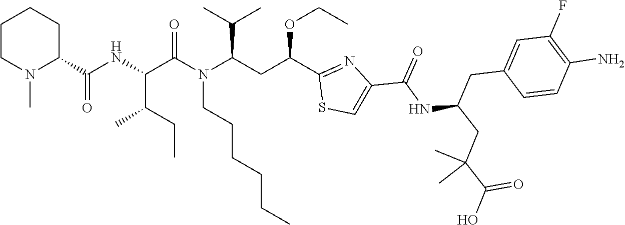 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C]

Following General Procedure VI from compound 3Ba with compound TUPa, payload P3 (23 mg, 70% yield) was obtained as a white solid. ESI m/z: 831.5 (M+H)⁺. ¹H NMR (400 MHz, methanol_{d4}) δ 7.96 (s, 1H), 6.71-6.58 (m, 3H), 4.56 (d, J=9.6 Hz, 1H), 4.28 (d, J=12.8 Hz, 1H), 4.24-4.17 (m, 1H), 3.78-3.68 (m, 1H), 3.62-3.55 (m, 1H), 3.49-3.35 (m, 2H), 3.10-3.07 (m, 2H), 2.88-2.82 (m, 1H), 2.62-2.60 (m, 2H), 2.11 (s, 3H), 1.94-1.78 (m, 5H), 1.77-1.70 (m, 4H), 1.53-1.41 (m, 4H), 1.24 (s, 3H), 1.23 (s, 3H), 1.18-1.17 (m, 4H), 1.14-1.11 (m, 3H), 1.02 (d, J=10.0 Hz, 6H), 0.89-0.87 (m, 6H), 0.82-0.79 (m, 6H), 0.72 (d, J=6.0 Hz, 3H) ppm.

### P5: (4S)-5-(4-amino-3-fluorophenyl)-4-({2-[(1R,3R)-1-{[(2-aminoethyl)carbamoyl]oxy}-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic Acid (P5)

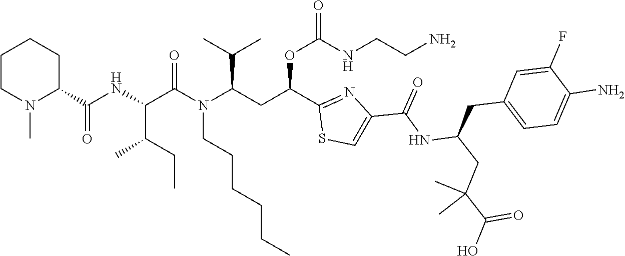 [SMILES: CCCCCCN(C(=O)[C@@H](NC(=O)[C@H]1CCCCN1C)[C@@H](C)CC)[C@H](C[C@@H](OC(=O)NCCN)c1nc(C(=O)N[C@@H](Cc2ccc(N)c(F)c2)CC(C)(C)C(=O)O)cs1)C(C)C]

Following General Procedure VI from 3Eb with TUPa, Boc-P5 (20 mg, ESI m/z: 445 (M/2+H)⁺) was obtained after purification by reversed phase flash chromatography (0-100% acetonitrile in water for 30 minutes and then 100% methanol for 20 minutes). To a suspension of Boc-P5 in DCM (3.6 mL) was added TFA (0.4 mL) and the mixture was stirred until clear. The resulting mixture was stirred for another 2 hours until Boc was totally removed, according to LCMS. The reaction mixture was concentrated in vacuo and the residue was purified by reversed phase flash chromatography (0-100% acetonitrile in water), and then by prep-HPLC (0-100% acetonitrile in aq. ammonium bicarbonate (10 mM)) to give payload P5 (9 mg, 19% yield from 3Eb) as a white solid. ESI m/z: 445 (M/2+H)⁺. ¹H NMR (400 MHz, DMSO_{d6}) δ 8.14 (s, 1H), 7.88 (br s, 2H), 7.75 (d, J=12.4 Hz, 1H), 6.68-6.61 (m, 2H), 5.56-5.53 (m, 1H), 4.94 (s, 2H), 4.47 (t, J=9.6 Hz, 1H), 4.19-4.14 (m, 1H), 3.73-3.65 (m, 1H), 3.07-2.92 (m, 3H), 2.84-2.55 (m, 5H), 2.17-1.73 (m, 10H), 1.61-1.41 (m, 7H), 1.36 (d, J=4.0 Hz, 2H), 1.33-1.27 (m, 7H), 1.20-1.02 (m, 9H), 0.94 (d, J=6.0 Hz, 3H), 0.85-0.79 (m, 11H), 0.69 (br s, 3H) ppm.

### P6: (4S)-5-(4-aminophenyl)-4-({2-[(1R,3R)-3-[(2S,3S)—N-hexyl-3-methyl-2-{[(2R)-1-methylpiperidin-2-yl]formamido}pentanamido]-1-hydroxy-4-methylpentyl]-1,3-thiazol-4-yl}formamido)-2,2-dimethylpentanoic Acid (P6)

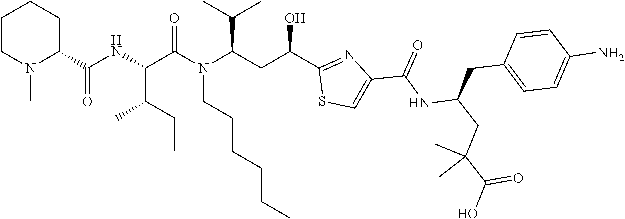 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)cc2)CC(C)(C)C(=O)O)cs1)C(C)C]

Following General Procedure VI from compound 3Aa (60 mg, crude) with compound TUPb, TBS-P6 was obtained. Without further purification, TBS-P6 was then dissolved in DMSO (3.0 mL). To the solution was added cesium fluoride (28 mg, 0.19 mmol), and the mixture was stirred at room temperature for 3 hours, and monitored by LCMS. The resulting mixture was filtered and the filtrate was purified by prep-HPLC (Method A) to give payload P6 (23 mg, 47% yield from 2Aa) as a white solid. ESI m/z: 393 (M/2+H)⁺. ¹H NMR (500 MHz, DMSO_{d6}) δ 8.07 (s, 1H), 7.92-7.66 (m, 1H), 7.51-7.20 (m, 1H), 6.79 (d, J=8.0 Hz, 2H), 6.44 (d, J=8.0 Hz, 2H), 6.32 (d, J=5.6 Hz, 1H), 4.99-4.77 (m, 2H), 4.64-4.43 (m, 2H), 4.43-4.12 (m, 1H), 3.76 (t, J=14.4 Hz, 1H), 3.10-2.93 (m, 1H), 2.91-2.77 (m, 1H), 2.06 (s, 3H), 2.02-1.72 (m, 6H), 1.64-1.40 (m, 7H), 1.38-1.23 (m, 8H), 1.19-1.07 (m, 2H), 1.03 (d, J=9.2 Hz, 7H), 0.92-0.75 (m, 15H), 0.72 (br s, 3H) ppm.

### P7: (4S)-4-({2-[(1R,3R)-1-(acetyloxy)-3-[(2S,3S)—N-hexyl-3-methyl-2-{[(2R)-1-methylpiperidin-2-yl]formamido}pentanamido]-4-methylpentyl]-1,3-thiazol-4-yl}formamido)-5-(4-aminophenyl)-2,2-dimethylpentanoic Acid (P7)

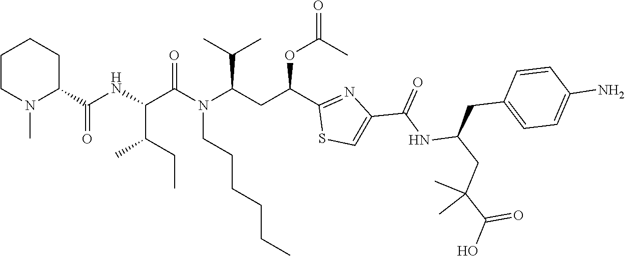 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C]

Following General Procedure VI from compound 3Fa with compound TUPb, payload P7 (4.0 mg, 50% yield from 3Fa) was obtained as a white solid. ESI m/z: 828 (M+H)⁺. ¹H NMR (400 MHz, DMSO_{d6}) δ 8.19 (s, 1H), 8.04 (s, 1H), 7.65 (d, J=8.9 Hz, 1H), 6.81 (d, J=8.1 Hz, 2H), 6.44 (d, J=8.2 Hz, 2H), 5.64 (d, J=13.0 Hz, 1H), 4.84 (s, 2H), 4.49 (t, J=9.3 Hz, 1H), 4.43-4.20 (m, 1H), 4.11 (s, 1H), 3.67 (d, J=14.8 Hz, 2H), 3.01 (d, J=11.0 Hz, 2H), 2.83 (d, J=11.4 Hz, 1H), 2.68 (d, J=4.7 Hz, 2H), 2.28 (dd, J=24.7, 12.1 Hz, 2H), 2.13 (s, 3H), 2.07 (s, 3H), 1.98-1.88 (m, 2H), 1.87-1.81 (m, 2H), 1.73 (s, 1H), 1.59 (s, 2H), 1.54 (s, 2H), 1.45 (s, 2H), 1.29 (s, 6H), 1.19-1.06 (m, 2H), 1.00 (d, J=9.8 Hz, 6H), 0.95 (d, J=6.4 Hz, 3H), 0.83 (dd, J=16.5, 9.4 Hz, 10H), 0.68 (d, J=5.8 Hz, 3H) ppm.

### P8: (4S)-5-(4-aminophenyl)-4-({2-[(1R,3R)-1-ethoxy-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic Acid (P8)

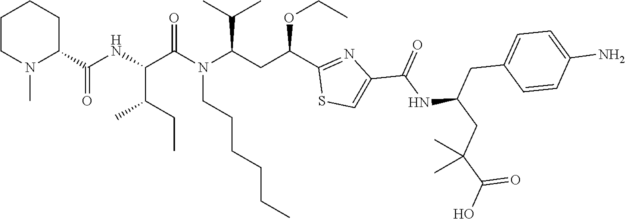 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)cc2)CC(C)(C)C(=O)O)cs1)C(C)C]

Following General Procedure VI from compound 3Ba with compound TUPb, payload P8 (16 mg, 23% yield) was obtained as a white solid. ESI m/z: 813.5 (M+H)⁺. ¹H NMR (400 MHz, DMSO_{d6}) δ 8.14 (s, 1H), 7.80-7.67 (br s, 1H), 7.48-7.41 (br s, 1H), 6.79 (d, J=8.4 Hz, 2H), 6.44 (d, J=8.0 Hz, 2H), 4.93-4.81 (br s, 2H), 4.51 (t, J=9.6 Hz, 1H), 4.34-4.28 (m, 1H), 4.17-4.12 (m, 1H), 3.79-3.71 (m, 2H), 3.03-2.94 (m, 2H), 2.86-2.83 (m, 1H), 2.64-2.59 (m, 2H), 2.08 (s, 3H), 1.99-1.74 (m, 7H), 1.68-1.37 (m, 9H), 1.33-1.23 (m, 9H), 1.17 (t, J=7.2 Hz, 3H), 1.03 (d, J=8.0 Hz, 6H), 0.91-0.82 (m, 12H), 0.74-0.65 (m, 3H) ppm.

### P9: (4S)-5-(4-aminophenyl)-4-({2-[(1R,3R)-1-acetamido-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic Acid (P9)

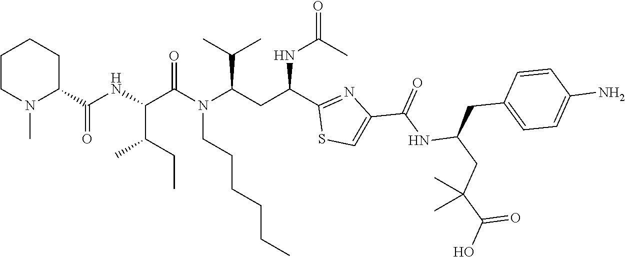 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(N)cc2)CC(C)(C)C(=O)O)cs1)C(C)C]

Following General Procedure VI from compound 3C with compound TUPb, payload P9 (6.4 mg, 12% yield from compound 3C) was obtained as a white sold after purification by prep-HPLC (Method A). ESI m/z: 826 (M+H)⁺. ¹H NMR (500 MHz, DMSO_{d6}) δ 8.66 (d, J=7.3 Hz, 1H), 8.03 (s, 1H), 7.58 (s, 1H), 7.39 (s, 1H), 6.81 (d, J=8.2 Hz, 2H), 6.45 (d, J=8.2 Hz, 2H), 4.91-4.80 (m, 2H), 4.46 (t, J=9.3 Hz, 1H), 4.20 (s, 1H), 3.68-3.62 (m, 1H), 3.01-2.58 (m, 4H), 2.15-1.98 (m, 5H), 1.97-1.71 (m, 9H), 1.68-1.40 (m, 6H), 1.40-1.16 (m, 9H), 1.10-1.00 (m, 8H), 0.97 (d, J=6.4 Hz, 3H), 0.92-0.73 (m, 10H), 0.68 (s, 3H) ppm.

### P10: (4S)-4-({2-[(1R,3R)-1-(acetyloxy)-3-[(2S,3S)—N-hexyl-3-methyl-2-{[(2R)-1-methylpiperidin-2-yl]formamido}pentanamido]-4-methylpentyl]-1,3-thiazol-4-yl}formamido)-5-(4-fluorophenyl)-2,2-dimethylpentanoic Acid (P10)

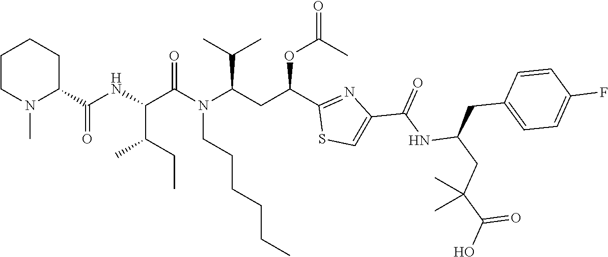 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)O)cs1)C(C)C]

Following General Procedure VI from compound 3Fa with compound TUPc, payload P10 (7.0 mg, 26% yield from 3Fa) was obtained as a white solid. ESI m/z: 830.5 (M+H)⁺. ¹H NMR (400 MHz, DMSO_{d6}) δ 8.16 (s, 1H), 7.75 (br s, 1H), 7.67 (d, J=9.6 Hz, 1H), 7.19 (dd, J=8.0 and 6.0 Hz, 2H), 7.06 (t, J=8.8 Hz, 2H), 5.64 (d, J=12.0 Hz, 1H), 4.48 (t, J=9.2 Hz, 1H), 4.27-4.23 (m, 1H), 3.73-3.65 (m, 1H), 3.02-2.93 (m, 1H), 2.84-2.75 (m, 3H), 2.33-1.83 (m, 11H), 1.90-1.40 (m, 8H), 1.28-1.23 (m, 11H), 1.17-1.13 (m, 1H), 1.06 (d, J=4.0 Hz, 6H), 0.96 (d, J=6.4 Hz, 3H), 0.87-0.79 (m, 9H), 0.68 (d, J=6.0 Hz, 3H) ppm.

### P1: (4S)-4-({2-[(1R,3R)-1-{[(2-{2-[2-(2-aminoethoxy)ethoxy]ethoxy}ethyl)carbamoyl]oxy}-3-[(2S,3S)—N-hexyl-3-methyl-2-{[(2R)-1-methylpiperidin-2-yl]formamido}pentanamido]-4-methylpentyl]-1,3-thiazol-4-yl}formamido)-5-(4-fluorophenyl)-2,2-dimethylpentanoic Acid (P11)

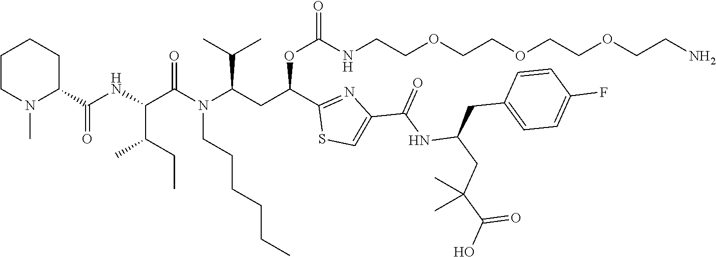 [SMILES: CCCCCCN(C(=O)[C@@H](NC(=O)[C@H]1CCCCN1C)[C@@H](C)CC)[C@H](C[C@@H](OC(=O)NCCOCCOCCOCCN)c1nc(C(=O)N[C@@H](Cc2ccc(F)cc2)CC(C)(C)C(=O)O)cs1)C(C)C]

Following General Procedure VI from compound 3Ec with compound TUPc, azido-P11 (40 mg, ESI m/z 1032 (M+H)⁺) was obtained after purification by reversed phase flash chromatography (0-100% methanol in aq. ammonium bicarbonate (10 mM)). Azido-P11 was dissolved in ethyl acetate (20 mL), and to the solution was added 10% palladium on carbon (40 mg) under nitrogen. The suspension was degassed and purged with hydrogen. The mixture was stirred at room temperature under a hydrogen balloon for 2 hours, and monitored by LCMS. The mixture was then filtered through Celite. The filtrate was concentrated and the residue was purified by reversed phase flash chromatography (0-100% methanol in aq. ammonium bicarbonate (10 mM)) to give payload P11 (28 mg, 20% yield from 3Ec) as a white solid. ESI m/z: 504 (M/2+H)⁺. ¹H NMR (400 MHz, DMSO_{d6}) δ 8.24-8.22 (m, 1H), 8.14 (s, 1H), 7.67-7.61 (m, 2H), 7.21-7.19 (m, 2H), 7.10-7.06 (m, 2H), 5.58-5.55 (m, 1H), 4.48 (t, J=9.2 Hz, 1H), 4.15 (br s, 1H), 3.83-3.69 (m, 10H), 3.35-3.32 (m, 2H), 3.23-3.17 (m, 1H), 3.04-2.94 (m, 3H), 2.87-2.82 (m, 2H), 2.74-2.67 (m, 3H), 2.15-2.12 (m, 1H), 2.08 (s, 3H), 2.03-1.61 (m, 6H), 1.63-1.37 (m, 8H), 1.31-1.24 (m, 9H), 1.17-1.05 (m, 2H), 1.01 (s, 3H), 0.97 (s, 3H), 0.94 (d, J=6.4 Hz, 3H), 0.87-0.81 (m, 10H), 0.71 (br s, 3H) ppm.

### P50: (4S)-4-({2-[(1R,3R)-1-ethoxy-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethyl-5-phenylpentanoic Acid (P50)

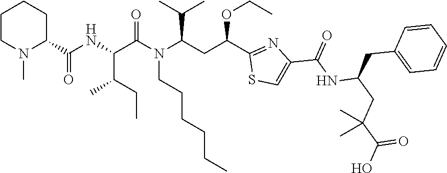 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccccc2)CC(C)(C)C(=O)O)cs1)C(C)C]

Following General Procedure VI from compound 3Ba with compound TUPe, P50 (30 mg, 60% yield from 3Ba) was obtained as a white solid after purification by reversed phase flash chromatography (0-100% methanol in aq. ammonium bicarbonate (10 mM)). ESI m/z: 798 (M+H)⁺.

TABLE 2-1
Compound List of Tubulysins Modified on MEP

| | | | | | | HPLC purity | HPLC RT |
|---|---|---|---|---|---|---|---|
| # | Structures | cLogP | MF | MW | Mass m/z | (%) | (min) |
| P12 | 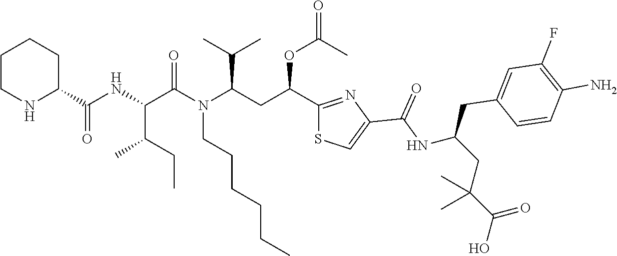 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] | 4.26 | C₄₃H₆₇FN₆O₇S | 831.1 | 416 (M/2 + H) | >99 | 7.28 (A) |
| P13 | 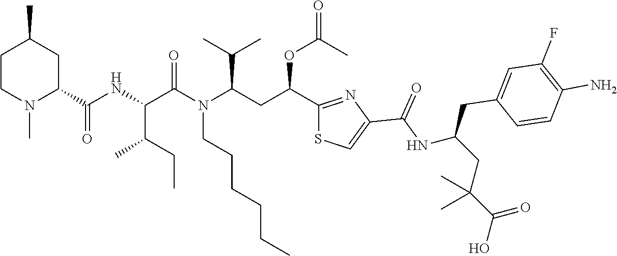 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] | 4.74 | C₄₅H₇₁FN₆O₇S | 859.2 | 430 (M/2 + H) | 96 | 7.39 (A) |
| P14 | 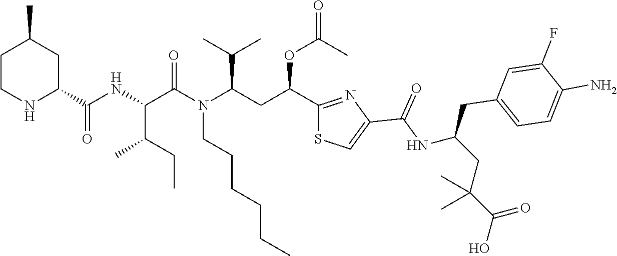 [SMILES: CCCCCCN(C(=O)[C@@H](NC(=O)[C@H]1C[C@H](C)CCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] | 4.54 | C₄₄H₆₉FN₆O₇S | 845.1 | 847 (M + H) | 99 | 7.57 (A) |
| P15 | 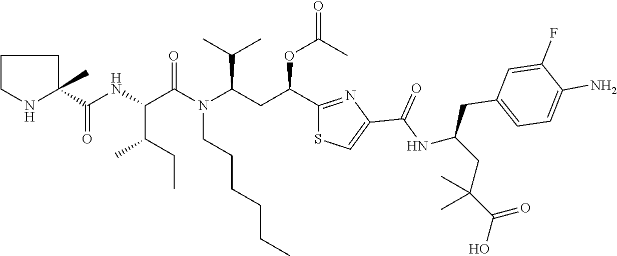 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] | 4.25 | C₄₃H₆₇FN₆O₇S | 831.1 | 416 (M/2 + H) | >99 | 7.51 (A) |
| P16 | 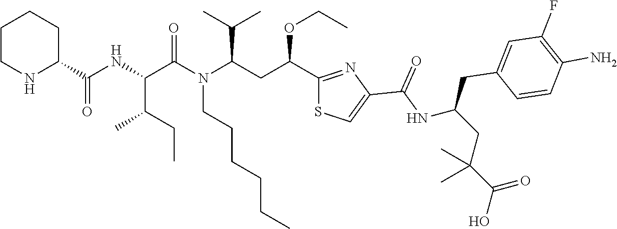 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] | 4.82 | C₄₃H₆₉FN₆O₆S | 817.1 | 817 (M + H) | 99 | 9.56 (B) |
| P17 | 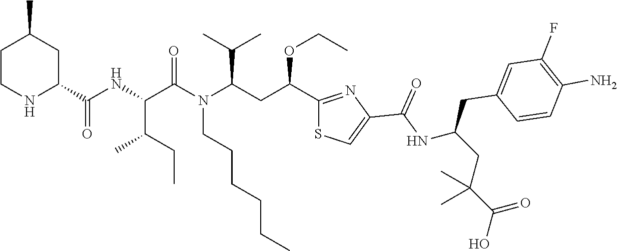 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] | 5.10 | C₄₄H₇₁FN₆O₆S | 831.1 | 831 (M + H) | 99 | 7.49 (A) |
| P18 | 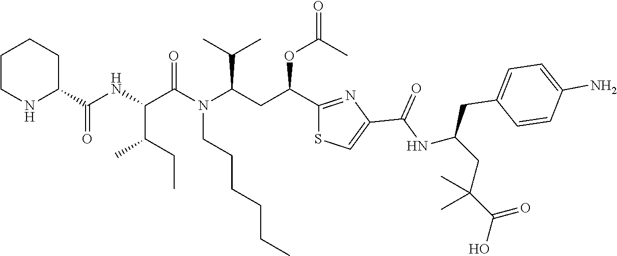 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] | 4.11 | C₄₃H₆₈N₆O₇S | 813.1 | 813 (M + H) | 99 | 8.88 (B) |
| P19 | 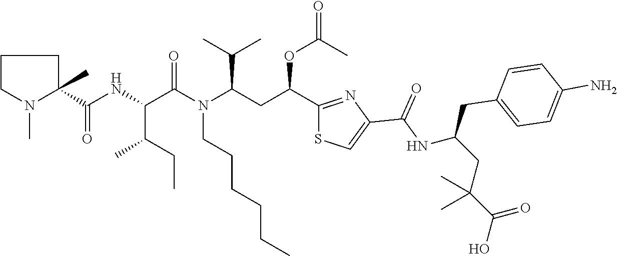 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] | 4.26 | C₄₄H₇₀N₆O₇S | 827.1 | 827 (M + H) | >99 | 8.90 (B) |
| P20 | 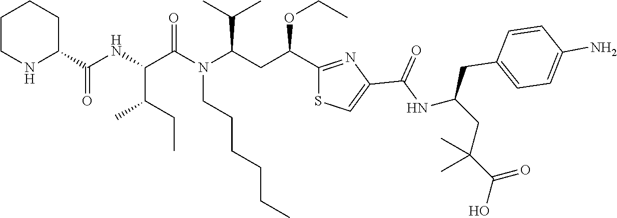 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)cc2)CC(C)(C)C(=O)O)cs1)C(C)C] | 4.66 | C₄₃H₇₀N₆O₆S | 799.1 | 799 (M + H) | >99 | 8.92 (B) |
| P21 | 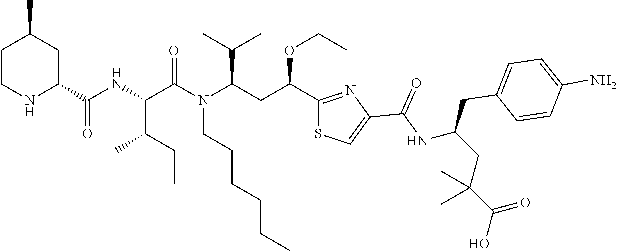 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)cc2)CC(C)(C)C(=O)O)cs1)C(C)C] | 4.95 | C₄₄H₇₂N₆O₆S | 813.2 | 814 (M + H) | 99 | 6.29 (A) |
| P22 | 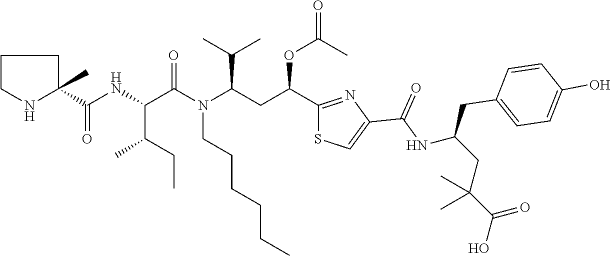 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C] | 4.63 | C₄₃H₆₇N₅O₈S | 814.1 | 815 (M + H) | >99 | 8.57 (B) |
| P23 | 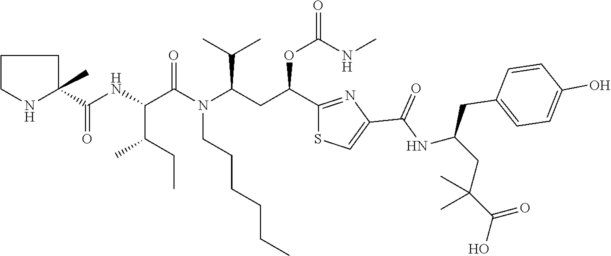 [SMILES: CCCCCCN(C(=O)[C@@H](NC(=O)[C@@]1(C)CCCN1)[C@@H](C)CC)[C@H](C[C@@H](OC(=O)NC)c1nc(C(=O)N[C@@H](Cc2ccc(O)cc2)CC(C)(C)C(=O)O)cs1)C(C)C] | 4.52 | C₄₃H₆₈N₆O₈S | 829.11 | 829 (M + H) | >99 | 8.93 (B) |

TABLE 2-2
Cytotoxicity of Tubulysin Payloads Modified on MEP

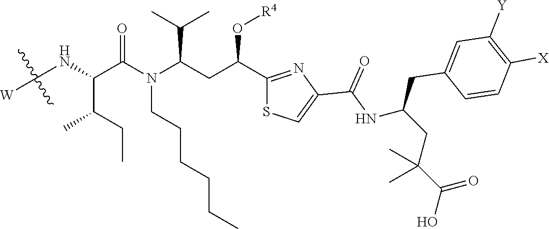

| | | | | | | HCT-15 with |
|---|---|---|---|---|---|---|
| | Structures | | | | HCT-15 | verapamil IC₅₀ |
| # | W | R⁴ | X | Y | IC₅₀ (nM) | (nM) |
| P12 | 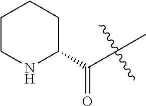 [SMILES: *C(=O)[C@H]1CCCCN1] | Ac | NH₂ | F | 0.34 | 0.03 |
| P13 | 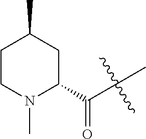 [SMILES: *C(=O)[C@H]1C[C@H](C)CCN1C] | Ac | NH₂ | F | 0.15 | 0.13 |
| P14 | 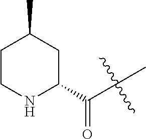 [SMILES: *C(=O)[C@H]1C[C@H](C)CCN1] | Ac | NH₂ | F | 1.21 | 0.10 |
| P15 | 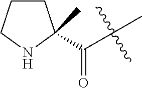 [SMILES: *C(=O)[C@@]1(C)CCCN1] | Ac | NH₂ | F | 1.01 | 0.19 |
| P3 | 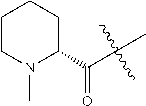 [SMILES: *C(=O)[C@H]1CCCCN1C] | Et | NH₂ | F | 0.07 | 0.01 |
| P16 | 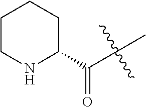 [SMILES: *C(=O)[C@H]1CCCCN1] | Et | NH₂ | F | 1.96 | 0.18 |
| P17 | 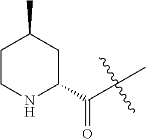 [SMILES: *C(=O)[C@H]1C[C@H](C)CCN1] | Et | NH₂ | F | 5.17 | 0.67 |
| P18 | 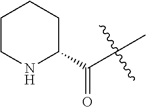 [SMILES: *C(=O)[C@H]1CCCCN1] | Ac | NH₂ | H | 0.98 | 0.05 |
| P19 | 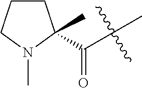 [SMILES: *C(=O)[C@@]1(C)CCCN1C] | Ac | NH₂ | H | 0.20 | 0.01 |
| P20 | 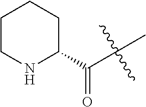 [SMILES: *C(=O)[C@H]1CCCCN1] | Et | NH₂ | H | 6.97 | 0.52 |
| P21 | 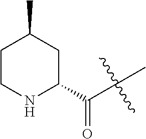 [SMILES: *C(=O)[C@H]1C[C@H](C)CCN1] | Et | NH₂ | H | 11.1 | 1.47 |
| P22 | 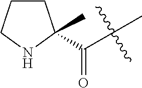 [SMILES: *C(=O)[C@@]1(C)CCCN1] | Ac | OH | H | 0.27 | 0.08 |
| P23 | 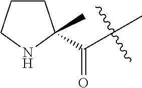 [SMILES: *C(=O)[C@@]1(C)CCCN1] | CONHMe | OH | H | 3.60 | 0.06 |

Synthesis of Intermediates 2A and 2B

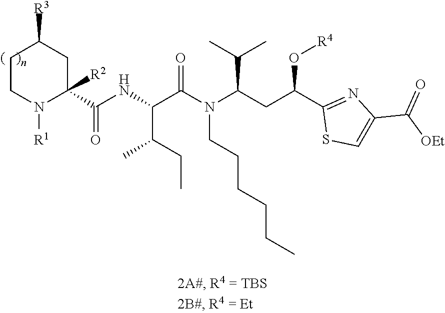

### Ethyl 2-[(1R,3R)-1-[(tert-butyldimethylsilyl)oxy]-3-[(2S,3S)—N-hexyl-3-methyl-2-{[(2R)-1-methylpiperidin-2-yl]formamido}pentanamido]-4-methylpentyl]-1,3-thiazole-4-carboxylate (2Aa)

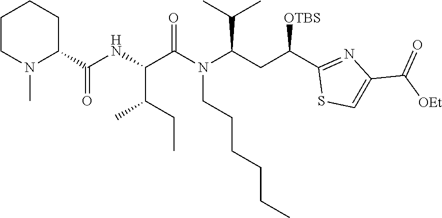 [SMILES: CCCCCCN(C(=O)[C@@H](NC(=O)[C@H]1CCCCN1C)[C@@H](C)CC)[C@H](C[C@@H](O[Si](C)(C)C(C)(C)C)c1nc(C(=O)OCC)cs1)C(C)C]

Following General Procedure I starting from intermediate 1A (54 mg, 92 μmol) with acid MEPa, crude compound 2Aa (60 mg, crude) was obtained as a white solid. ESI m/z: 710 (M+H)⁺.

### tert-Butyl (2R)-2-{[(1S,2S)-1-{[(1R,3R)-1-[(tert-butyldimethylsilyl)oxy]-1-[4-(ethoxycarbonyl)-1,3-thiazol-2-yl]-4-methylpentan-3-yl](hexyl)carbamoyl}-2-methylbutyl]carbamoyl}piperidine-1-carboxylate (2Ab)

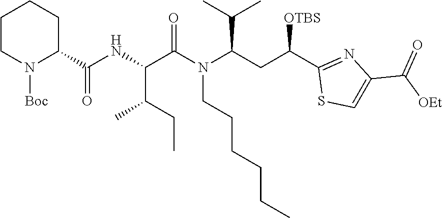 [SMILES: CCCCCCN(C(=O)[C@@H](NC(=O)[C@H]1CCCCN1C(=O)OC(C)(C)C)[C@@H](C)CC)[C@H](C[C@@H](O[Si](C)(C)C(C)(C)C)c1nc(C(=O)OCC)cs1)C(C)C]

Following General Procedure I starting from intermediate 1A with acid MEPb, crude compound 2Ab (0.30 g) was obtained as a white solid. ESI m/z: 795.5 (M+H)⁺.

### Ethyl 2-[(1R,3R)-1-[(tert-butyldimethylsilyl)oxy]-3-[(2S,3S)-2-{[(2R,4R)-1,4-dimethylpiperidin-2-yl]formamido}-N-hexyl-3-methylpentanamido]-4-methylpentyl]-1,3-thiazole-4-carboxylate (2Ac)

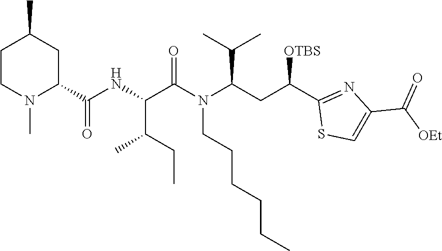 [SMILES: CCCCCCN(C(=O)[C@@H](NC(=O)[C@H]1C[C@H](C)CCN1C)[C@@H](C)CC)[C@H](C[C@@H](O[Si](C)(C)C(C)(C)C)c1nc(C(=O)OCC)cs1)C(C)C]

Following General Procedure I starting from intermediate 1A with acid MEPc, crude compound 2Ac (0.28 g) was obtained as a white solid. ESI m/z: 723 (M+H)⁺.

### tert-Butyl (2R,4R)-2-{[(1S,2S)-1-{[(1R,3R)-1-[(tert-butyldimethylsilyl)oxy]-1-[4-(ethoxycarbonyl)-1,3-thiazol-2-yl]-4-methylpentan-3-yl](hexyl)carbamoyl}-2-methylbutyl]carbamoyl}-4-methylpiperidine-1-carboxylate (2Ad)

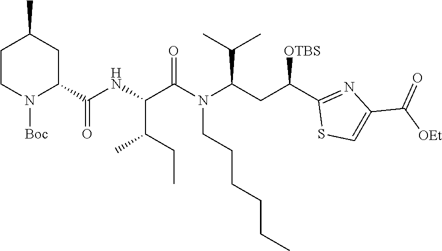 [SMILES: CCCCCCN(C(=O)[C@@H](NC(=O)[C@H]1C[C@H](C)CCN1C(=O)OC(C)(C)C)[C@@H](C)CC)[C@H](C[C@@H](O[Si](C)(C)C(C)(C)C)c1nc(C(=O)OCC)cs1)C(C)C]

Following General Procedure I starting from intermediate 1A (0.10 g, 0.17 mmol) with acid MEPd, compound 2Ad (0.10 g, 72% yield) was obtained as a white solid after purification by reversed phase flash chromatography (0-100% acetonitrile in water). ESI m/z: 809.5 (M+H)⁺.

### Ethyl 2-[(1R,3R)-3-[(2S,3S)-2-{[(2R)-1-[(tert-butoxy)carbonyl]-2-methylpyrrolidin-2-yl]formamido}-N-hexyl-3-methylpentanamido]-1-[(tert-butyldimethylsilyl)oxy]-4-methylpentyl]-1,3-thiazole-4-carboxylate (2Ae)

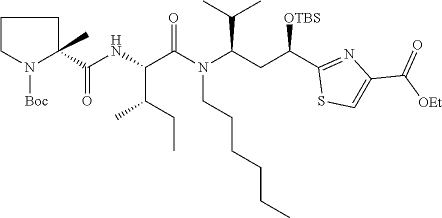 [SMILES: CCCCCCN(C(=O)[C@@H](NC(=O)[C@@]1(C)CCCN1C(=O)OC(C)(C)C)[C@@H](C)CC)[C@H](C[C@@H](O[Si](C)(C)C(C)(C)C)c1nc(C(=O)OCC)cs1)C(C)C]

Following General Procedure I starting from intermediate 1A with acid MEPe, crude compound 2Ae (0.30 g, crude) was obtained as a white solid. ESI m/z: 795.5 (M+H)⁺.

### Ethyl 2-[(1R,3R)-1-ethoxy-3-[(2S,3S)—N-hexyl-3-methyl-2-{[(2R)-1-methylpiperidin-2-yl]formamido}pentanamido]-4-methylpentyl]-1,3-thiazole-4-carboxylate (2Ba)

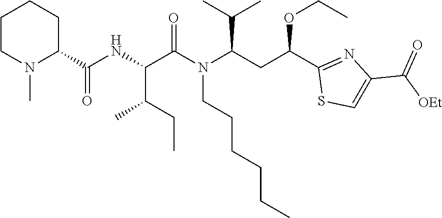 [SMILES: CCCCCCN(C(=O)[C@@H](NC(=O)[C@H]1CCCCN1C)[C@@H](C)CC)[C@H](C[C@@H](OCC)c1nc(C(=O)OCC)cs1)C(C)C]

Following General Procedure I starting from intermediate 1B (50 mg, 0.10 mmol) with acid MEPa, compound 2Ba (31 mg, 50% yield) was obtained as a white solid after purification by prep-HPLC (Method B). ESI m/z: 623 (M+H)⁺.

### tert-Butyl (2R)-2-{[(1S,2S)-1-{[(1R,3R)-1-ethoxy-1-[4-(ethoxycarbonyl)-1,3-thiazol-2-yl]-4-methylpentan-3-yl](hexyl)carbamoyl}-2-methylbutyl]carbamoyl}piperidine-1-carboxylate (2Bb)

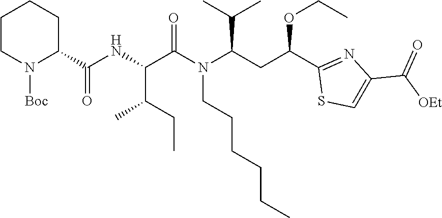 [SMILES: CCCCCCN(C(=O)[C@@H](NC(=O)[C@H]1CCCCN1C(=O)OC(C)(C)C)[C@@H](C)CC)[C@H](C[C@@H](OCC)c1nc(C(=O)OCC)cs1)C(C)C]

Following General Procedure I starting from intermediate 1B (50 mg, 0.10 mmol) with acid MEPb, compound 2Bb (60 mg, 84% yield) was obtained as a white solid after purification by prep-HPLC (Method B). ESI m/z: 709 (M+H)⁺.

### tert-Butyl (2R,4R)-2-{[(1S,2S)-1-{[(1R,3R)-1-ethoxy-1-[4-(ethoxycarbonyl)-1,3-thiazol-2-yl]-4-methylpentan-3-yl](hexyl)carbamoyl}-2-methylbutyl]carbamoyl}-4-methylpiperidine-1-carboxylate (2Bc)

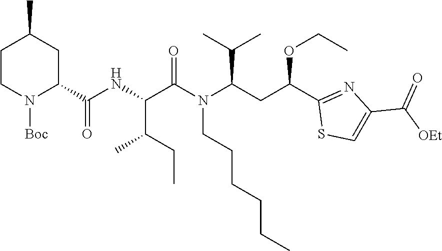 [SMILES: CCCCCCN(C(=O)[C@@H](NC(=O)[C@H]1C[C@H](C)CCN1C(=O)OC(C)(C)C)[C@@H](C)CC)[C@H](C[C@@H](OCC)c1nc(C(=O)OCC)cs1)C(C)C]

Following General Procedure I starting from intermediate 1B (0.10 g, 0.20 mmol) with acid MEPd, compound 2Bc (0.10 g, 69% yield) was obtained as a white solid after purification by prep-HPLC (Method B). ESI m/z: 724 (M+H)⁺.

Synthesis of Intermediate 2D

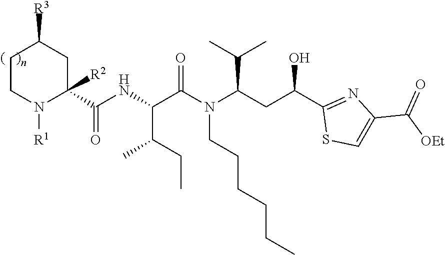

### Ethyl 2-[(1R,3R)-3-[(2S,3S)—N-hexyl-3-methyl-2-{[(2R)-1-methylpiperidin-2-yl]formamido}pentanamido]-1-hydroxy-4-methylpentyl]-1,3-thiazole-4-carboxylate (2 Da)

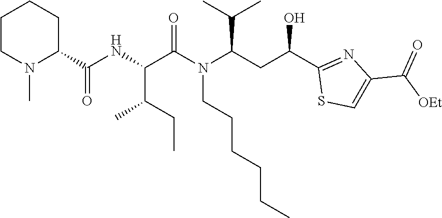 [SMILES: CCCCCCN(C(=O)[C@@H](NC(=O)[C@H]1CCCCN1C)[C@@H](C)CC)[C@H](C[C@@H](O)c1nc(C(=O)OCC)cs1)C(C)C]

Following General Procedure II starting from crude compound 2Aa (0.50 g) in DMSO (6 mL), compound 2 Da (0.32 g, 75% yield in 2 steps) was obtained as a light yellow oil. ESI m/z: 595 (M+H)⁺.

### tert-Butyl (2R)-2-{[(1S,2S)-1-{[(1R,3R)-1-[4-(ethoxycarbonyl)-1,3-thiazol-2-yl]-1-hydroxy-4-methylpentan-3-yl](hexyl)carbamoyl}-2-methylbutyl]carbamoyl}piperidine-1-carboxylate (2Db)

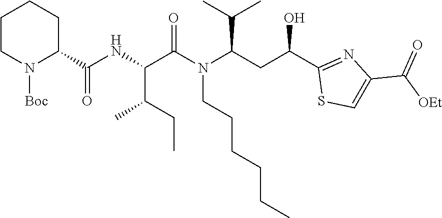 [SMILES: CCCCCCN(C(=O)[C@@H](NC(=O)[C@H]1CCCCN1C(=O)OC(C)(C)C)[C@@H](C)CC)[C@H](C[C@@H](O)c1nc(C(=O)OCC)cs1)C(C)C]

Following General Procedure II starting from crude compound 2Ab, compound 2Db (0.21 g, 99% yield) was obtained as an off-white solid. ESI m/z: 681 (M+H)⁺.

### Ethyl 2-[(1R,3R)-3-[(2S,3S)-2-{[(2R,4R)-1,4-dimethylpiperidin-2-yl]formamido}-N-hexyl-3-methylpentanamido]-1-hydroxy-4-methylpentyl]-1,3-thiazole-4-carboxylate (2Dc)

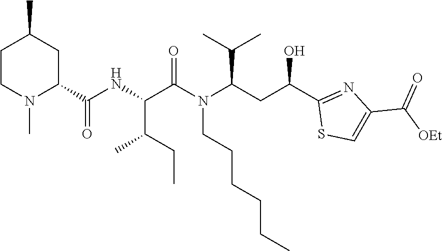 [SMILES: CCCCCCN(C(=O)[C@@H](NC(=O)[C@H]1C[C@H](C)CCN1C)[C@@H](C)CC)[C@H](C[C@@H](O)c1nc(C(=O)OCC)cs1)C(C)C]

Following General Procedure II starting from crude compound 2Ac (0.21 g, 0.35 mmol), compound 2Dc (0.21 g, 99% yield in 2 steps) was obtained as an off-white solid. ESI m/z: 609 (M+H)⁺.

### tert-Butyl (2R,4R)-2-{[(1S,2S)-1-{[(1R,3R)-1-[4-(ethoxycarbonyl)-1,3-thiazol-2-yl]-1-hydroxy-4-methylpentan-3-yl](hexyl)carbamoyl}-2-methylbutyl]carbamoyl}-4-methylpiperidine-1-carboxylate (2Dd)

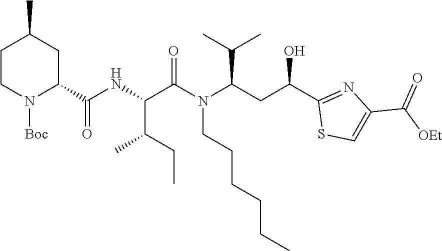 [SMILES: CCCCCCN(C(=O)[C@@H](NC(=O)[C@H]1C[C@H](C)CCN1C(=O)OC(C)(C)C)[C@@H](C)CC)[C@H](C[C@@H](O)c1nc(C(=O)OCC)cs1)C(C)C]

Following General Procedure II starting from compound 2Ad (0.10 g, 0.12 mmol), compound 2Dd (75 mg, 87% yield) was obtained as a white solid. ESI m/z: 695 (M+H)⁺.

### Ethyl 2-[(1R,3R)-3-[(2S,3S)-2-{[(2R)-1-[(tert-butoxy)carbonyl]-2-methylpyrrolidin-2-yl]formamido}-N-hexyl-3-methylpentanamido]-1-hydroxy-4-methylpentyl]-1,3-thiazole-4-carboxylate (2De)

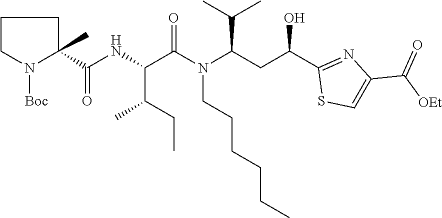 [SMILES: CCCCCCN(C(=O)[C@@H](NC(=O)[C@@]1(C)CCCN1C(=O)OC(C)(C)C)[C@@H](C)CC)[C@H](C[C@@H](O)c1nc(C(=O)OCC)cs1)C(C)C]

Following General Procedure II starting from crude compound 2Ae (0.30 g), compound 2De (0.18 g, 90% yield in 2 steps) was obtained as an off-white solid. ESI m/z: 681 (M+H)⁺.

Synthesis of Intermediate 3B

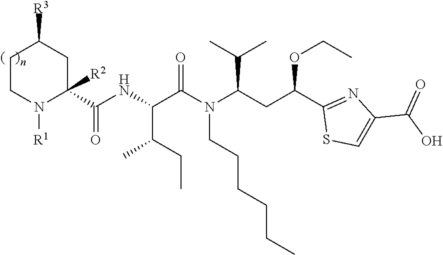

### 2-[(1R,3R)-1-Ethoxy-3-[(2S,3S)—N-hexyl-3-methyl-2-{[(2R)-1-methylpiperidin-2-yl]formamido}pentanamido]-4-methylpentyl]-1,3-thiazole-4-carboxylic Acid (3Ba)

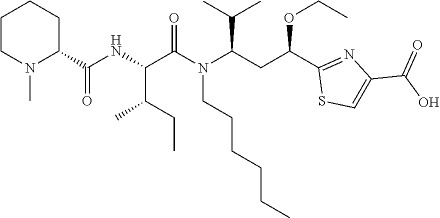 [SMILES: CCCCCCN(C(=O)[C@@H](NC(=O)[C@H]1CCCCN1C)[C@@H](C)CC)[C@H](C[C@@H](OCC)c1nc(C(=O)O)cs1)C(C)C]

Following General Procedure IV from 2Ba (62 mg, 0.10 mmol), acid 3Ba (46 mg, 80% yield) was obtained as a white solid after purification by reversed phase flash chromatography (5-100% acetonitrile in aq. TFA (0.03%)). ESI m/z: 595 (M+H)⁺.

### 2-[(1R,3R)-3-[(2S,3S)-2-{[(2R)-1-[(tert-Butoxy)carbonyl]piperidin-2-yl]formamido}-N-hexyl-3-methylpentanamido]-1-ethoxy-4-methylpentyl]-1,3-thiazole-4-carboxylic Acid (3Bb)

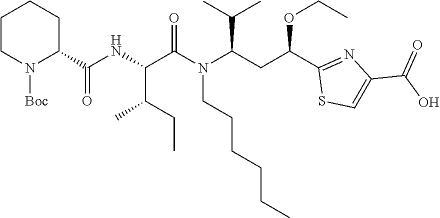 [SMILES: CCCCCCN(C(=O)[C@@H](NC(=O)[C@H]1CCCCN1C(=O)OC(C)(C)C)[C@@H](C)CC)[C@H](C[C@@H](OCC)c1nc(C(=O)O)cs1)C(C)C]

Following General Procedure IV from 2Bb (60 mg, 85 μmol), acid 3Bb (35 mg, 57% yield) was obtained as a white solid after purification by reversed phase flash chromatography (5-100% acetonitrile in aq. ammonium bicarbonate (10 mM)). ESI m/z: 681 (M+H)⁺.

### 2-[(1R,3R)-3-[(2S,3S)-2-{[(2R,4R)-1-[(tert-Butoxy)carbonyl]-4-methylpiperidin-2-yl]formamido}-N-hexyl-3-methylpentanamido]-1-ethoxy-4-methylpentyl]-1,3-thiazole-4-carboxylic Acid (3Bc)

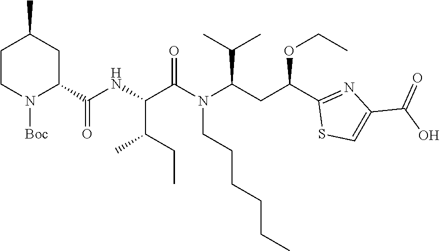 [SMILES: CCCCCCN(C(=O)[C@@H](NC(=O)[C@H]1C[C@H](C)CCN1C(=O)OC(C)(C)C)[C@@H](C)CC)[C@H](C[C@@H](OCC)c1nc(C(=O)O)cs1)C(C)C]

Following General Procedure IV from 2Bc (0.10 g, 89 μmol), acid 3Bc (64 mg, 71% yield) was obtained as a white solid after purification by reversed phase flash chromatography (0-30% acetonitrile in water). ESI m/z: 695 (M+H)⁺. ¹H NMR (400 MHz, DMSO_{d6}) δ 8.31 (s, 1H), 7.98 (d, J=9.6 Hz, 1H), 4.62-4.57 (m, 2H), 4.32-4.29 (m, 1H), 3.90-3.82 (m, 1H), 3.81-3.73 (m, 1H), 3.52-3.48 (m, 1H), 3.46-3.42 (m, 1H), 3.10-3.01 (m, 1H), 2.14-2.05 (m, 1H), 1.97-1.84 (m, 4H), 1.61-1.52 (m, 2H), 1.49-1.42 (m, 1H), 1.37 (s, 5H), 1.32 (m, 9H), 1.27-1.24 (m, 1H), 1.16-1.12 (m, 5H), 0.92-0.80 (m, 20H), 0.74-0.69 (m, 3H) ppm.

Synthesis of Intermediate 3D

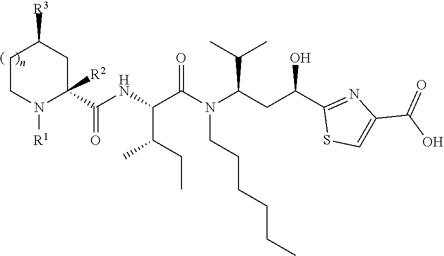

### 2-[(1R,3R)-3-[(2S,3S)—N-Hexyl-3-methyl-2-{[(2R)-1-methylpiperidin-2-yl]formamido}pentanamido]-1-hydroxy-4-methylpentyl]-1,3-thiazole-4-carboxylic Acid (3 Da)

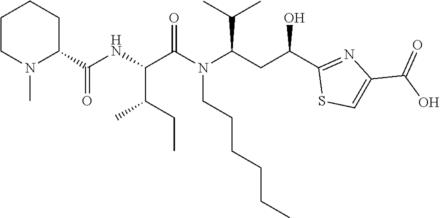 [SMILES: CCCCCCN(C(=O)[C@@H](NC(=O)[C@H]1CCCCN1C)[C@@H](C)CC)[C@H](C[C@@H](O)c1nc(C(=O)O)cs1)C(C)C]

Following General Procedure IV from 2 Da (0.15 g, 0.24 mmol), crude acid 3 Da (0.14 g, 94% yield) was obtained as an off-white solid, and used in the next step without further purification. ESI m/z: 567 (M+H)⁺.

### 2-[(1R,3R)-3-[(2S,3S)-2-{[(2R)-1-[(tert-Butoxy)carbonyl]piperidin-2-yl]formamido}-N-hexyl-3-methylpentanamido]-1-hydroxy-4-methylpentyl]-1,3-thiazole-4-carboxylic Acid (3Db)

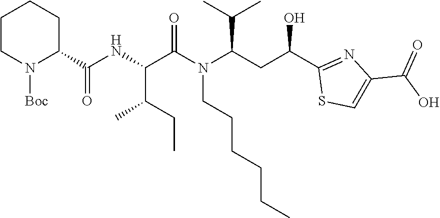 [SMILES: CCCCCCN(C(=O)[C@@H](NC(=O)[C@H]1CCCCN1C(=O)OC(C)(C)C)[C@@H](C)CC)[C@H](C[C@@H](O)c1nc(C(=O)O)cs1)C(C)C]

Following General Procedure IV from 2Db (0.21 g, 0.31 mmol), crude acid 3Db (0.18 g, 89% crude yield) was obtained as an off-white solid, and used in the next step without further purification. ESI m/z: 653 (M+H)⁺.

### 2-[(1R,3R)-3-[(2S,3S)-2-{[(2R,4R)-1,4-Dimethylpiperidin-2-yl]formamido}-N-hexyl-3-methylpentanamido]-1-hydroxy-4-methylpentyl]-1,3-thiazole-4-carboxylic Acid (3Dc)

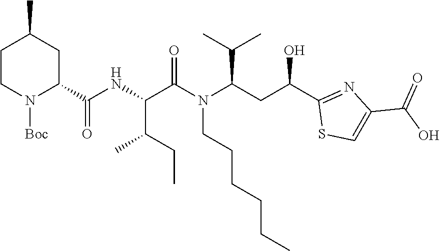 [SMILES: CCCCCCN(C(=O)[C@@H](NC(=O)[C@H]1C[C@H](C)CCN1C(=O)OC(C)(C)C)[C@@H](C)CC)[C@H](C[C@@H](O)c1nc(C(=O)O)cs1)C(C)C]

Following General Procedure IV from 2Dc (0.21 g, 0.35 mmol), crude acid 3Dc (0.18 g, 89% crude yield) was obtained as an off-white solid, and used in the next step without further purification. ESI m/z: 580 (M+H)⁺.

### 2-[(1R,3R)-3-[(2S,3S)-2-{[(2R,4R)-1-[(tert-Butoxy)carbonyl]-4-methylpiperidin-2-yl]formamido}-N-hexyl-3-methylpentanamido]-1-hydroxy-4-methylpentyl]-1,3-thiazole-4-carboxylic Acid (3Dd)

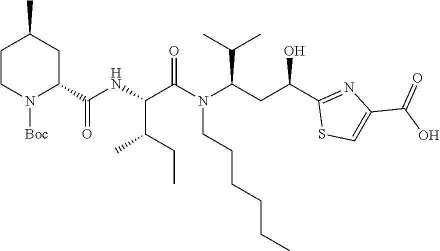 [SMILES: CCCCCCN(C(=O)[C@@H](NC(=O)[C@H]1C[C@H](C)CCN1C(=O)OC(C)(C)C)[C@@H](C)CC)[C@H](C[C@@H](O)c1nc(C(=O)O)cs1)C(C)C]

Following General Procedure IV from 2Dd (75 mg, 0.11 mmol), acid 3Dd (50 mg, 69% yield) was obtained as a white solid after purification by reversed phase flash chromatography (0-70% acetonitrile in water). ESI m/z: 689 (M+Na)⁺, 567 (M−Boc+H)⁺.

### 2-[(1R,3R)-3-[(2S,3S)-2-{[(2R)-1-[(tert-Butoxy)carbonyl]-2-methylpyrrolidin-2-yl]formamido}-N-hexyl-3-methylpentanamido]-1-hydroxy-4-methylpentyl]-1,3-thiazole-4-carboxylic Acid (3De)

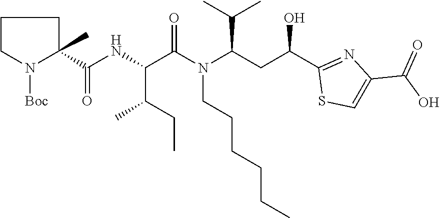 [SMILES: CCCCCCN(C(=O)[C@@H](NC(=O)[C@@]1(C)CCCN1C(=O)OC(C)(C)C)[C@@H](C)CC)[C@H](C[C@@H](O)c1nc(C(=O)O)cs1)C(C)C]

Following General Procedure IV from 2De (75 mg, 0.11 mmol), crude acid 3De (66 mg, 92% yield) was obtained as an off-white solid, and used in the next step without further purification. ESI m/z: 653 (M+H)⁺.

Synthesis of Intermediate 3Ed

### 2-[(1R,3R)-3-[(2S,3S)-2-{[(2R)-1-[(tert-butoxy)carbonyl]-2-methylpyrrolidin-2-yl]formamido}-N-hexyl-3-methylpentanamido]-4-methyl-1-[(methylcarbamoyl)oxy]pentyl]-1,3-thiazole-4-carboxylic Acid (3Ed)

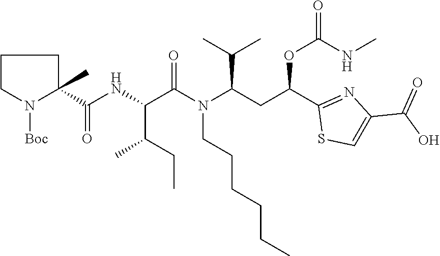 [SMILES: CCCCCCN(C(=O)[C@@H](NC(=O)[C@@]1(C)CCCN1C(=O)OC(C)(C)C)[C@@H](C)CC)[C@H](C[C@@H](OC(=O)NC)c1nc(C(=O)O)cs1)C(C)C]

Successively following General Procedure III and IV starting from 2De (0.10 g, 0.15 mmol), acid 3Ed (63 mg, 68% yield) was obtained as an off-white solid, and used in the next step without further purification. ESI m/z 710 (M+H)⁺. ¹H NMR (400 MHz, DMSO_{d6}) δ 12.98 (s, 1H), 8.38 (s, 1H), 7.37 (d, J=4.4 Hz, 1H), 7.25-7.21 (m, 1H), 5.56-5.52 (m, 1H), 4.48-4.46 (m, 1H), 3.63 (br s, 1H), 3.47 (br s, 1H), 3.17-2.67 (m, 2H), 2.55 (d, J=4.4 Hz, 3H), 2.15-2.11 (m, 1H), 2.06-1.99 (m, 1H), 1.93-1.58 (m, 8H), 1.51-1.31 (m, 19H), 1.08-1.02 (m, 1H), 0.92 (d, J=6.4 Hz, 3H), 0.88-0.78 (m, 10H), 0.70 (br s, 3H) ppm.

Synthesis of Intermediate 3F

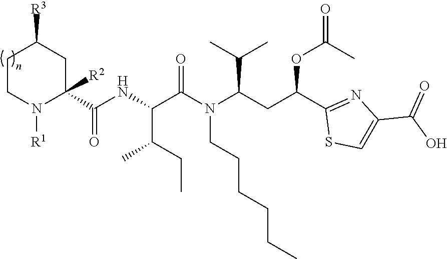

### 2-[(1R,3R)-1-(Acetyloxy)-3-[(2S,3S)—N-hexyl-3-methyl-2-{[(2R)-1-methylpiperidin-2-yl]formamido}pentanamido]-4-methylpentyl]-1,3-thiazole-4-carboxylic Acid (3Fa)

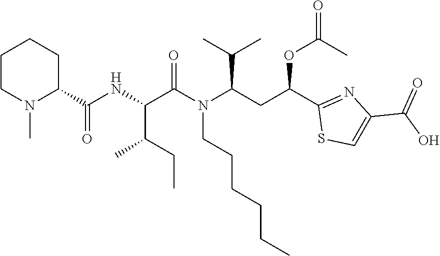 [SMILES: CCCCCCN(C(=O)[C@@H](NC(=O)[C@H]1CCCCN1C)[C@@H](C)CC)[C@H](C[C@@H](OC(C)=O)c1nc(C(=O)O)cs1)C(C)C]

Following General Procedure V from compound 3 Da (0.13 g, 0.22 mmol), acid 3Fa (0.12 g, 90% yield) was obtained as a white solid after purification by reversed phase flash chromatography (0-25% acetonitrile in aq. ammonium bicarbonate (0.08%)). ESI m/z: 609 (M+H)⁺.

### 2-[(1R,3R)-1-(Acetyloxy)-3-[(2S,3S)-2-{[(2R)-1-[(tert-butoxy)carbonyl]piperidin-2-yl]formamido}-N-hexyl-3-methylpentanamido]-4-methylpentyl]-1,3-thiazole-4-carboxylic Acid (3Fb)

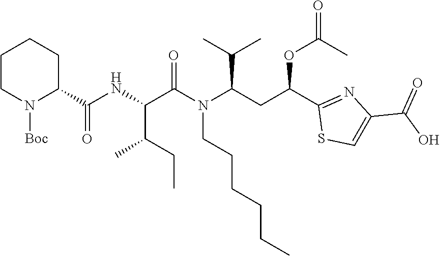 [SMILES: CCCCCCN(C(=O)[C@@H](NC(=O)[C@H]1CCCCN1C(=O)OC(C)(C)C)[C@@H](C)CC)[C@H](C[C@@H](OC(C)=O)c1nc(C(=O)O)cs1)C(C)C]

Following General Procedure V from compound 3Db (0.18 g, 0.28 mmol), acid 3Fb (0.18 g, 94% yield) was obtained as a white solid after purification by reversed phase flash chromatography (0-25% acetonitrile in aq. ammonium bicarbonate (0.08%)). ESI m/z: 695 (M+H)⁺.

### 2-[(1R,3R)-1-(Acetyloxy)-3-[(2S,3S)-2-{[(2R,4R)-1,4-dimethylpiperidin-2-yl]formamido}-N-hexyl-3-methylpentanamido]-4-methylpentyl]-1,3-thiazole-4-carboxylic Acid (3Fc)

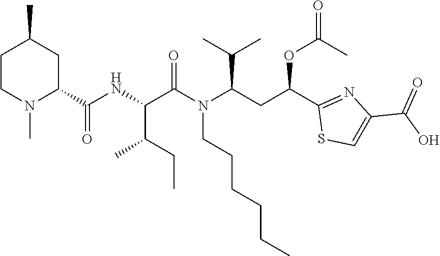 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)O)cs1)C(C)C]

Following General Procedure V from compound 3Dc (0.18 g, 0.31 mmol), acid 3Fc (0.17 g, 88% yield) was obtained as a white solid after purification by reversed phase flash chromatography (0-50% acetonitrile in aq. ammonium bicarbonate (10 mM)). ESI m/z: 623 (M+H)⁺.

### 2-[(1R,3R)-1-(Acetyloxy)-3-[(2S,3S)-2-{[(2R,4R)-1-[(tert-Butoxy)carbonyl]-4-methylpiperidin-2-yl]formamido}-N-hexyl-3-methylpentanamido]-4-methylpentyl]-1,3-thiazole-4-carboxylic Acid (3Fd)

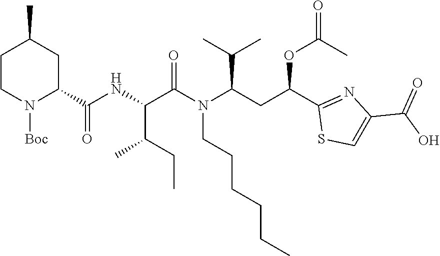 [SMILES: CCCCCCN(C(=O)[C@@H](NC(=O)[C@H]1C[C@H](C)CCN1C(=O)OC(C)(C)C)[C@@H](C)CC)[C@H](C[C@@H](OC(C)=O)c1nc(C(=O)O)cs1)C(C)C]

Following General Procedure V from compound 3Dd (50 mg, 75 μmol), acid 3Fd (42 mg, 45% yield) was obtained as a white solid after purification by reversed phase flash chromatography (0-75% acetonitrile in aq. ammonium bicarbonate (0.08%)). ESI m/z: 709 (M+H)⁺, 609 (M−Boc+H)⁺, 731 (M+Na)⁺. ¹H NMR (400 MHz, DMSO_{d6}) δ 8.24 (s, 1H), 7.69 (s, 1H), 7.52 (s, 1H), 5.64 (d, J=12.0 Hz, 1H), 4.62-4.47 (m, 2H), 3.90-3.81 (m, 1H), 3.80-3.72 (m, 1H), 3.30 (s, 1H), 3.05-2.99 (m, 1H), 2.34-2.28 (m, 1H), 2.22-2.15 (m, 1H), 2.01 (s, 3H), 2.07-1.96 (m, 1H), 1.93-1.87 (m, 1H), 1.57-1.51 (m, 2H), 1.48-1.44 (m, 1H), 1.38 (s, 4H), 1.32 (s, 7H), 1.30-1.26 (m, 5H), 1.24-1.22 (m, 1H), 0.97-0.93 (m, 4H), 0.87-0.65 (m, 18H) ppm.

### 2-[(1R,3R)-1-(Acetyloxy)-3-[(2S,3S)-2-{[(2R)-1-[(tert-butoxy)carbonyl]-2-methylpyrrolidin-2-yl]formamido}-N-hexyl-3-methylpentanamido]-4-methylpentyl]-1,3-thiazole-4-carboxylic Acid (3Fe)

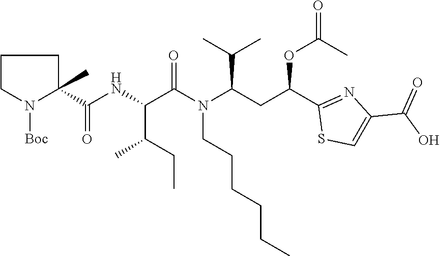 [SMILES: CCCCCCN(C(=O)[C@@H](NC(=O)[C@@]1(C)CCCN1C(=O)OC(C)(C)C)[C@@H](C)CC)[C@H](C[C@@H](OC(C)=O)c1nc(C(=O)O)cs1)C(C)C]

Following General Procedure V from compound 3De (66 mg, 0.10 mmol), acid 3Fe (50 mg, 71% yield) was obtained as a white solid after purification by reversed phase flash chromatography (0-100% acetonitrile in aq. ammonium bicarbonate (10 mM)). ESI m/z: 695 (M+H)⁺. ¹H NMR (400 MHz, DMSO_{d6}) δ 13.11 (s, 1H), 8.45 (s, 1H), 7.40-7.32 (m, 1H), 5.63 (d, J=13.2 Hz, 1H), 4.51-4.45 (m, 1H), 4.41-4.40 (m, 1H), 3.75-3.42 (m, 2H), 3.36-3.29 (m, 1H), 3.04-2.89 (m, 1H), 2.27-2.20 (m, 1H), 2.11-2.08 (m, 4H), 2.02-1.51 (m, 9H), 1.46-1.43 (m, 3H), 1.39-1.36 (m, 9H), 1.34-1.28 (m, 6H), 1.07-0.98 (m, 1H), 0.93 (d, J=6.4 Hz, 3H), 0.88-0.82 (m, 9H), 10.67 (d, J=4.4 Hz, 3H) ppm.

### 2-[(1R,3R)-1-(Acetyloxy)-3-[(2S,3S)-2-{[(2R)-1,2-dimethylpyrrolidin-2-yl]formamido}-N-hexyl-3-methylpentanamido]-4-methylpentyl]-1,3-thiazole-4-carboxylic Acid (3Ff)

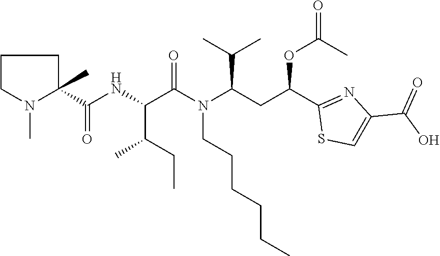 [SMILES: CCCCCCN(C(=O)[C@@H](NC(=O)[C@@]1(C)CCCN1C)[C@@H](C)CC)[C@H](C[C@@H](OC(C)=O)c1nc(C(=O)O)cs1)C(C)C]

To a solution of compound 3Fe (0.40 g, 0.58 mmol) in DCM (3 mL) was added TFA (1 mL), and the mixture was stirred at room temperature for 3 hours until Boc was totally removed, according to LCMS. The mixture was concentrated in vacuo and the residue was purified by reversed phase flash chromatography (10-30% acetonitrile in water) to give the intermediate (0.32 g, 78% yield, TFA salt, ESI m/z 595 (M+H)⁺) as a white solid.

To a solution of the intermediate (50 mg, 84 μmol) in methanol (2 mL) and H₂O (2 mL) was added paraformaldehyde (76 mg, 0.84 mmol), and the mixture was stirred at room temperature for 10 minutes before the addition of 10% palladium on charcoal (50 mg) under nitrogen. The resulting suspension was degassed, purged with hydrogen 3 times, stirred under hydrogen atmosphere at room temperature overnight, and monitored by LCMS. The reaction mixture was then filtered through Celite and the filtrate was concentrated in vacuo to give compound 3Ff (36 mg, 71% yield) as a white solid. ESI m/z 609 (M+H)⁺.

Synthesis of Tubulysin Payloads in Table 2

### P12: (4S)-4-({2-[(1R,3R)-1-(acetyloxy)-3-[(2S,3S)—N-hexyl-3-methyl-2-[(2R)-piperidin-2-ylformamido]pentanamido]-4-methylpentyl]-1,3-thiazol-4-yl}formamido)-5-(4-amino-3-fluorophenyl)-2,2-dimethylpentanoic Acid (P12)

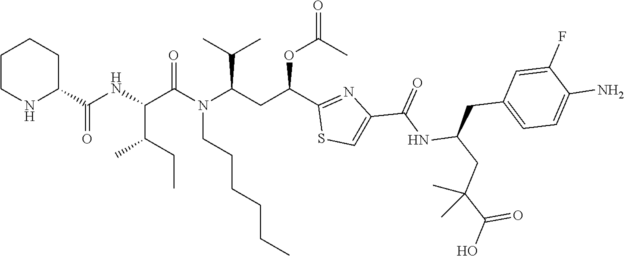 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C]

Following General Procedure VI from compound 3Fb (0.10 g, 0.14 mmol) with compound TUPa, Boc-P12 (30 mg, ESI m/z: 416 (M/2+H)⁺) was obtained as a white solid, and was dissolved into DCM (3 mL). To the solution was added TFA (1 mL), and the reaction mixture was stirred at room temperature for 4 hours until Boc was totally removed according to LCMS. The resulting mixture was concentrated in vacuo and the residue was purified by prep-HPLC (5-100% acetonitrile in aq. formic acid (0.1%)) to give P12 (8.8 mg, 7.5% yield from 3Fb) as a white solid. ESI m/z 831.4 (M+H)⁺. ¹H NMR (400 MHz, DMSO_{d6}) δ 8.37 (s, 1H), 8.16 (s, 1H), 7.76-7.60 (m, 2H), 6.75 (d, J=12.4 Hz, 1H), 6.68-6.59 (m, 2H), 5.66 (d, J=12.8 Hz, 1H), 4.93-4.89 (m, 2H), 4.49 (t, J=9.2 Hz, 1H), 4.21 (s, 1H), 3.80-3.67 (m, 2H), 3.22-3.17 (m, 2H), 3.10-3.02 (m, 2H), 2.87-2.82 (m, 1H), 2.67-2.56 (m, 2H), 2.32-2.23 (m, 2H), 2.14 (s, 3H), 1.84-1.80 (m, 3H), 1.70-1.60 (m, 4H), 1.49-1.44 (m, 2H), 1.36-1.20 (m, 8H), 1.06-1.05 (m, 7H), 0.95 (d, J=6.8 Hz, 3H), 0.88-0.73 (m, 10H), 0.65 (d, J=6.0 Hz, 3H) ppm. ¹⁹F NMR (376 MHz, DMSO_{d6}) 6-135.5 ppm.

### P13: (4S)-4-({2-[(1R,3R)-1-(acetyloxy)-3-[(2S,3S)-2-{[(2R,4R)-1,4-dimethylpiperidin-2-yl]formamido}-N-hexyl-3-methylpentanamido]-4-methylpentyl]-1,3-thiazol-4-yl}formamido)-5-(4-amino-3-fluorophenyl)-2,2-dimethylpentanoic Acid (P13)

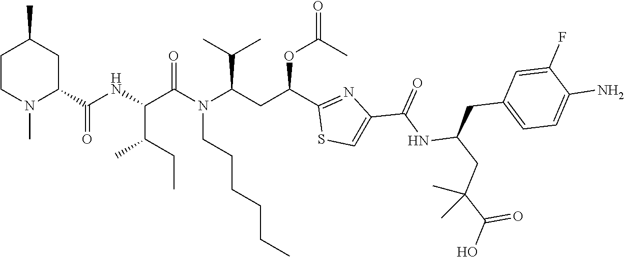 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C]

Following General Procedure VI from compound 3Fc with compound TUPa, P13 (11 mg, 13% yield) was obtained as a white solid. ESI m/z: 430 (M/2+H)⁺. ¹H NMR (400 MHz, DMSO_{d6}) δ 8.17 (s, 1H), 7.90 (s, 1H), 6.75 (d, J=12.0 Hz, 1H), 6.67-6.60 (m, 2H), 5.65 (d, J=12.4 Hz, 1H), 4.94 (s, 2H), 4.48 (t, J=9.6 Hz, 1H), 4.20 (s, 1H), 3.78-3.71 (m, 1H), 3.22-3.13 (m, 1H), 2.98-2.87 (m, 2H), 2.66-2.58 (m, 2H), 2.39-2.32 (m, 2H), 2.3 (s, 4H), 2.14 (s, 3H), 1.91-1.82 (m, 3H), 1.66-1.60 (m, 3H), 1.48-1.42 (m, 3H), 1.33-1.24 (m, 9H), 1.18-1.01 (m, 8H), 0.95 (d, J=6.4 Hz, 3H), 0.85-0.79 (m, 13H), 0.70 (d, J=4.8 Hz, 3H) ppm. ¹⁹F NMR (376 MHz, DMSO_{d6}) 6-135.5 ppm.

### P14: (4S)-4-({2-[(1R,3R)-1-(acetyloxy)-3-[(2S,3S)—N-hexyl-3-methyl-2-{[(2R,4R)-4-methylpiperidin-2-yl]formamido}pentanamido]-4-methylpentyl]-1,3-thiazol-4-yl}formamido)-5-(4-amino-3-fluorophenyl)-2,2-dimethylpentanoic Acid (P14)

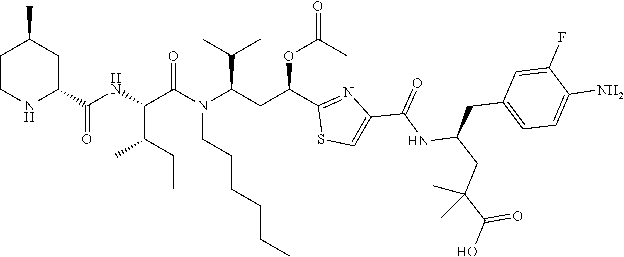 [SMILES: CCCCCCN(C(=O)[C@@H](NC(=O)[C@H]1C[C@H](C)CCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C]

Following General Procedure VI from compound 3Fd (21 mg, 30 μmol) with compound TUPa, Boc-P14 (15 mg, ESI m/z: 946 (M+H)⁺) was obtained as a white solid after purification by reversed phase flash chromatography (0-60% acetonitrile in water). Boc-P14 (15 mg) was dissolved in DCM (3 mL) and to the solution was added TFA (1 mL). The reaction mixture was stirred at room temperature for 3 hours until Boc was totally removed according to LCMS. The resulting mixture was concentrated in vacuo and the residue was purified by prep-HPLC (10-95% acetonitrile in aq. formic acid (0.1%)) to give P14 (8.1 mg, 32% yield from 3Fd) as a white solid. ESI m/z 847 (M+H)⁺, 423 (M/2+H)⁺. ¹H NMR (400 MHz, DMSO_{d6}) δ 8.50 (s, 1H), 8.17 (s, 1H), 7.58-7.50 (m, 1H), 6.75 (d, J=12.8 Hz, 1H), 6.67-6.60 (m, 2H), 5.68-5.63 (m, 1H), 4.92 (s, 2H), 4.54 (t, J=9.6 Hz, 1H), 4.26-4.18 (m, 1H), 3.7-3.72 (m, 1H), 3.66 (t, J=11.6 Hz, 1H), 3.09-2.98 (m, 2H), 2.97-2.79 (m, 3H), 2.64-2.58 (m, 2H), 2.34-2.21 (m, 2H), 2.15 (s, 3H), 1.92-1.79 (m, 5H), 1.69-1.63 (m, 2H), 1.60-1.52 (m, 2H), 1.49-1.43 (m, 1H), 1.34-1.21 (m, 7H), 1.09-1.04 (m, 7H), 0.98-0.93 (m, 6H), 0.89-0.78 (m, 10H), 0.71 (d, J=6.4 Hz, 3H) ppm.

### P15: (4S)-4-({2-[(1R,3R)-1-(acetyloxy)-3-[(2S,3S)—N-hexyl-3-methyl-2-{[(2R)-2-methylpyrrolidin-2-yl]formamido}pentanamido]-4-methylpentyl]-1,3-thiazol-4-yl}formamido)-5-(4-amino-3-fluorophenyl)-2,2-dimethylpentanoic Acid (P15)

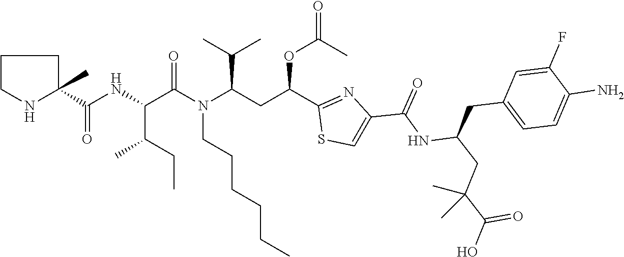 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C]

Following General Procedure VI from compound 3Fe (100 mg, 0.14 mmol) with compound TUPa, Boc-P15 (30 mg, ESI m/z: 931.5 (M+H)⁺) was obtained as an off-white solid after purification by reversed phase flash chromatography (0-30% acetonitrile in aq. ammonium bicarbonate (10 mM)). Boc-P15 (30 mg) was dissolved in DCM (3 mL) and to the solution was added TFA (1 mL). The reaction mixture was stirred at room temperature for 4 hours until Boc was totally removed according to LCMS. The resulting mixture was concentrated in vacuo and the residue was purified by prep-HPLC (0-100% acetonitrile in aq. formic acid (0.1%)) to give P15 (8.8 mg, 7.6% yield from 3Ff) as a white solid. ESI m/z 416 (M/2+H)⁺. ¹H NMR (400 MHz, DMSO_{d6}) δ 8.17 (s, 1H), 8.12 (d, J=10.4 Hz, 1H), 7.61-7.56 (m, 1H), 6.75 (d, J=12.4 Hz, 1H), 6.68-6.59 (m, 2H), 5.65 (d, J=11.6 Hz, 1H), 4.95 (s, 2H), 4.41 (t, J=9.6 Hz, 1H), 4.21 (s, 1H), 3.80-3.67 (m, 2H), 3.22-3.17 (m, 2H), 3.10-3.02 (m, 2H), 2.87-2.82 (m, 1H), 2.67-2.56 (m, 2H), 2.33-2.22 (m, 2H), 2.14 (s, 3H), 1.84-1.80 (m, 3H), 1.70-1.60 (m, 4H), 1.49-1.44 (m, 2H), 1.36-1.20 (m, 8H), 1.06-1.05 (m, 7H), 0.95 (d, J=6.8 Hz, 3H), 0.88-0.73 (m, 10H), 0.65 (d, J=5.6 Hz, 3H) ppm. ¹⁹F NMR (376 MHz, DMSO_{d6}) δ −135.5 ppm.

### P16: (4S)-5-(4-amino-3-fluorophenyl)-4-({2-[(1R,3R)-1-ethoxy-3-[(2S,3S)—N-hexyl-3-methyl-2-[(2R)-piperidin-2-ylformamido]pentanamido]-4-methylpentyl]-1,3-thiazol-4-yl}formamido)-2,2-dimethylpentanoic Acid (P16)

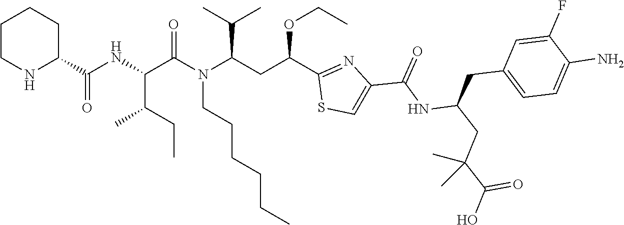 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)c(F)c2)CC(C)(C)C(=O)O)cs1)C(C)C]

Following General Procedure VI for payloads from compound 3Bb (35 mg, 51 mol) with compound TUPa, Boc-P16 (50 mg, ESI m/z: 917.5 (M+H)⁺) was obtained as a yellow oil. Boc-P16 was dissolved in DCM (4 mL). To the solution was added TFA (1 mL) and the reaction mixture was stirred at room temperature for an hour until Boc was totally removed according to LCMS. The resulting mixture was concentrated in vacuo and the residue was purified by prep-HPLC (0-100% acetonitrile in aq. TFA (0.1%)) to give P16 (10 mg, 21% yield from 3Bb, dual-TFA salt) as a white solid. ESI m/z: 817 (M+H)⁺. ¹H NMR (400 MHz, DMSO_{d6}) δ 12.03 (br s, 1H), 8.84 (d, J=9.2 Hz, 2H), 8.66 (d, J=9.9 Hz, 1H), 8.16 (s, 1H), 7.42 (s, 1H), 6.73 (d, J=13.0 Hz, 1H), 6.69-6.52 (m, 2H), 4.92 (s, 2H), 4.60 (t, J=13.2 Hz, 1H), 4.32 (d, J=10.6 Hz, 1H), 4.22-4.20 (m, 1H), 3.74 (s, 1H), 3.68-3.55 (m, 2H), 3.22-2.90 (m, 5H), 2.64-2.54 (m, 2H), 2.27-2.21 (m, 2H), 2.12-1.37 (m, 13H), 1.40-1.22 (m, 7H), 1.18 (t, J=6.8 Hz, 3H), 1.06 (d, J=5.1 Hz, 6H), 0.94-0.78 (m, 13H), 0.74 (d, J=6.1 Hz, 3H) ppm. ¹⁹F NMR (376 MHz, DMSO_{d6}) 6-73.5, −135.4 ppm.

### P17: (4S)-5-(4-amino-3-fluorophenyl)-4-({2-[(1R,3R)-1-ethoxy-3-[(2S,3S)—N-hexyl-3-methyl-2-{[(2R,4R)-4-methylpiperidin-2-yl]formamido}pentanamido]-4-methylpentyl]-1,3-thiazol-4-yl}formamido)-2,2-dimethylpentanoic Acid (P17)

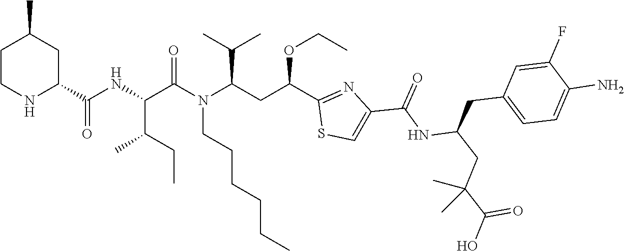 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)c(F)c2)CC(C)(C)C(=O)O)cs1)C(C)C]

Following General Procedure VI for payloads from compound 3Bc (32 mg, 46 mol) with compound TUPa, Boc-P17 (25 mg, ESI m/z: 931.5 (M+H)⁺) was obtained as a white solid. Boc-P17 was dissolved in DCM (3 mL). To the solution was added TFA (1 mL), and the mixture was stirred at room temperature for 3 hours until Boc was totally removed according to LCMS. The resulting mixture was concentrated in vacuo and the residue was purified by prep-HPLC (5-90% acetonitrile in aq. formic acid (0.01%)) to give P17 (9.7 mg, 26% yield from 3Bc) as a white solid. ESI m/z: 831 (M+H)⁺. ¹H NMR (400 MHz, DMSO_{d6}) δ 8.19-8.16 (m, 1H), 6.74 (d, J=12.8 Hz, 1H), 6.67-6.60 (m, 2H), 4.96-4.90 (m, 2H), 4.58-4.51 (m, 1H), 4.32-4.28 (m, 1H), 4.23-4.14 (m, 1H), 3.08-2.99 (m, 3H), 2.94-2.87 (m, 2H), 2.81-2.74 (m, 1H), 2.65-2.59 (m, 2H), 2.00-1.82 (m, 7H), 1.64-1.53 (m, 4H), 1.51-1.46 (m, 1H), 1.33-1.25 (m, 6H), 1.20-1.15 (m, 4H), 1.13-1.11 (m, 1H), 1.07 (s, 3H), 1.05 (s, 3H), 0.94-0.80 (m, 19H), 0.77-0.70 (m, 3H) ppm. ¹⁹F NMR (376 MHz, DMSO_{d6}) 6-135.4 ppm.

### P18: (4S)-4-({2-[(1R,3R)-1-(acetyloxy)-3-[(2S,3S)—N-hexyl-3-methyl-2-[(2R)-piperidin-2-ylformamido]pentanamido]-4-methylpentyl]-1,3-thiazol-4-yl}formamido)-5-(4-aminophenyl)-2,2-dimethylpentanoic Acid (P18)

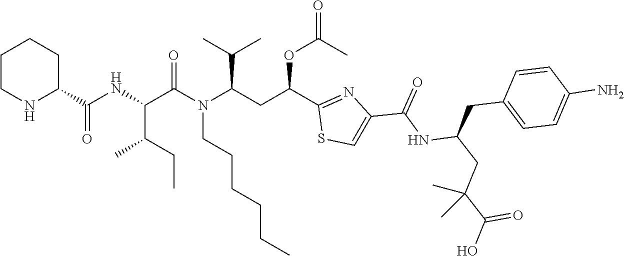 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C]

Following General Procedure VI for payloads from compound 3Fb (20 mg, 29 mol) with compound TUPb, Boc-P18 (15 mg, ESI m/z: 913 (M+H)⁺) was obtained as a white solid after purification by prep-HPLC (5-95% acetonitrile in aq. TFA (0.01%)). To a solution of Boc-P18 (15 mg) in DCM (0.6 mL) was added TFA (0.2 mL), and the mixture was stirred at room temperature for 3 hours until Boc was totally removed according to LCMS. The resulting mixture was concentrated in vacuo and the residue was purified by prep-HPLC (0-100% acetonitrile in aq. ammonium bicarbonate (10 mM)) to give P18 (4.2 mg, 18% yield from 3Fb) as a white solid. ESI m/z: 813 (M+H)⁺. ¹H NMR (400 MHz, DMSO_{d6}) δ 8.36 (s, 1H), 8.16 (s, 1H), 7.81-7.54 (m, 2H), 6.80 (d, J=8.3 Hz, 2H), 6.44 (d, J=8.3 Hz, 2H), 5.65 (d, J=13.3 Hz, 1H), 4.98-4.71 (m, 2H), 4.48 (t, J=9.5 Hz, 1H), 4.25-4.08 (m, 2H), 3.02-2.94 (m, 2H), 2.90-2.80 (m, 2H), 2.68-2.59 (m, 1H), 2.30-2.21 (m, 2H), 2.14 (s, 3H), 2.03-1.95 (m, 2H), 1.86-1.79 (m, 2H), 1.71-1.55 (m, 4H), 1.49-1.41 (m, 2H), 1.34-1.21 (m, 12H), 1.04 (s, 3H), 1.03 (s, 3H), 0.96 (d, J=6.4 Hz, 3H), 0.88-0.79 (m, 9H), 0.69 (d, J=6.2 Hz, 3H) ppm. >99.9% ee via an R′R WHELK column.

### P19: (4S)-4-({2-[(1R,3R)-1-(acetyloxy)-3-[(2S,3S)-2-{[(2R)-1,2-dimethylpyrrolidin-2-yl]formamido}-N-hexyl-3-methylpentanamido]-4-methylpentyl]-1,3-thiazol-4-yl}formamido)-5-(4-aminophenyl)-2,2-dimethylpentanoic Acid (P19)

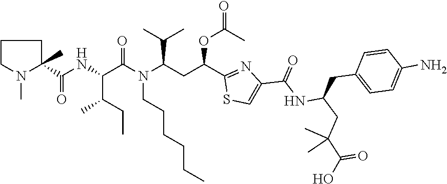 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C]

Following General Procedure VI for payloads from compound 3Ff (36 mg, 59 mol) with compound TUPb, P19 (3.2 mg, 6.7% yield) was obtained as a white solid after purification by prep-HPLC (5-95% acetonitrile in aq. TFA (0.1%)). ESI m/z: 827 (M+H)⁺. ¹H NMR (500 MHz, DMSO_{d6}) δ 8.43 (s, 1H), 8.17 (s, 1H), 7.75-7.72 (d, J=10.4 Hz, 1H), 7.66 (s, 1H), 6.81-6.79 (d, J=8.0 Hz, 2H), 6.45-6.43 (d, J=8.0 Hz, 2H), 5.66-5.63 (d, J=8.8 Hz, 1H), 4.86 (s, 2H), 4.48-4.42 (d, J=9.2 Hz, 1H), 4.17 (s, 1H), 3.62-3.54 (m, 1H), 3.06-2.96 (m, 2H), 2.68-2.55 (m, 2H), 2.45-2.41 (m, 2H), 2.33-2.27 (m, 1H), 2.21 (s, 3H), 2.13 (s, 3H), 1.85-1.88 (m, 1H), 1.77-1.75 (m, 4H), 1.62-1.54 (m, 3H), 1.51-1.45 (m, 3H), 1.29-1.24 (m, 6H), 1.08 (s, 3H), 1.03-1.02 (d, J=3.6 Hz, 7H), 0.96-0.95 (d, J=6.4 Hz, 3H), 0.88-0.79 (m, 10H), 0.68-0.66 (d, J=6.0 Hz, 3H) ppm.

### P20: (4S)-5-(4-aminophenyl)-4-({2-[(1R,3R)-1-ethoxy-3-[(2S,3S)—N-hexyl-3-methyl-2-[(2R)-piperidin-2-ylformamido]pentanamido]-4-methylpentyl]-1,3-thiazol-4-yl}formamido)-2,2-dimethylpentanoic Acid (P20)

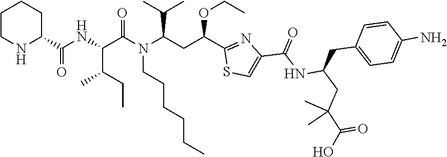 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)cc2)CC(C)(C)C(=O)O)cs1)C(C)C]

Following General Procedure VI for payloads from compound 3Bb (35 mg, 51 mol) with compound TUPb, Boc-P20 (50 mg, ESI m/z: 899 (M+H)⁺) was obtained as a yellow oil. Boc-P20 was dissolved in DCM (4 mL). To the solution was added TFA (1 mL), and the reaction mixture was stirred at room temperature for an hour, and monitored by LCMS. The resulting mixture was concentrated in vacuo and the residue was purified by prep-HPLC (0-100% acetonitrile in aq. ammonium bicarbonate (10 mM)) to give P20 (9.1 mg, 22% yield from 3Bb) as a white solid. ESI m/z: 799 (M+H)⁺. ¹H NMR (500 MHz, DMSO_{d6}) δ 8.38 (s, 1H), 8.15 (s, 1H), 6.79 (d, J=8.1 Hz, 2H), 6.44 (d, J=8.1 Hz, 2H), 4.56-4.20 (m, 6H), 3.05-2.89 (m, 5H), 2.70-2.60 (m, 2H), 1.99-1.78 (m, 5H), 1.65-1.40 (m, 8H), 1.30-1.16 (m, 9H), 1.15-1.10 (m, 4H), 1.03-1.00 (m, 6H), 0.91-0.81 (m, 14H), 0.72 (s, 3H) ppm.

### P21: (4S)-5-(4-aminophenyl)-4-({2-[(1R,3R)-1-ethoxy-3-[(2S,3S)—N-hexyl-3-methyl-2-{[(2R,4R)-4-methylpiperidin-2-yl]formamido}pentanamido]-4-methylpentyl]-1,3-thiazol-4-yl}formamido)-2,2-dimethylpentanoic Acid (P21)

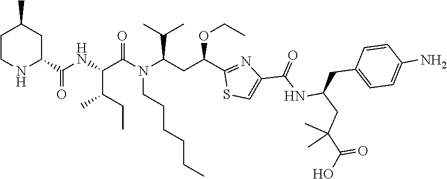 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)cc2)CC(C)(C)C(=O)O)cs1)C(C)C]

Following General Procedure VI for payloads from compound 3Bc (32 mg, 46 mol) with compound TUPb, Boc-P21 (25 mg, ESI m/z: 914 (M+H)⁺) was obtained as a white solid. Boc-P21 was dissolved in DCM (3 mL). To the solution was added TFA (1 mL), and the reaction mixture was stirred at room temperature for 3 hours until Boc was totally removed according to LCMS. The resulting mixture was concentrated in vacuo and the residue was purified by prep-HPLC (10-95% acetonitrile in aq. formic acid (0.01%)) to give P21 (11 mg, 29% yield) as a white solid. ESI m/z: 407 (M/2+H)⁺. ¹H NMR (400 MHz, DMSO_{d6}) δ 8.86-8.74 (m, 1H), 8.16 (s, 1H), 8.15 (s, 1H), 8.79 (d, J=8.4 Hz, 2H), 8.44 (d, J=8.0 Hz, 2H), 4.62-4.54 (m, 1H), 4.34-4.29 (m, 1H), 4.22-4.14 (m, 1H), 4.98-3.88 (m, 1H), 3.72-3.63 (m, 1H), 3.57-3.53 (m, 1H), 3.52-3.46 (m, 2H), 3.10-3.00 (m, 4H), 2.64-2.57 (m, 1H), 2.55-2.52 (m, 1H), 2.00-1.83 (m, 7H), 1.80-1.72 (m, 3H), 1.68-1.62 (m, 1H), 1.50-1.44 (m, 1H), 1.36-1.26 (m, 7H), 1.20-1.16 (m, 3H), 1.13-1.09 (m, 1H), 1.06-0.98 (m, 10H), 0.94-0.92 (m, 3H), 0.90-0.85 (m, 7H), 0.84-0.79 (m, 4H), 0.77-0.72 (m, 3H) ppm.

### P22: (4S)-4-({2-[(1R,3R)-1-(acetyloxy)-3-[(2S,3S)—N-hexyl-3-methyl-2-{[(2R)-2-methylpyrrolidin-2-yl]formamido}pentanamido]-4-methylpentyl]-1,3-thiazol-4-yl}formamido)-5-(4-hydroxyphenyl)-2,2-dimethylpentanoic Acid (P22)

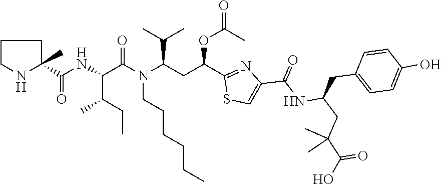 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C]

Following General Procedure VI for payloads from compound 3Fd (49 mg, 70 mol) with compound TUPd, Boc-P22 (22 mg, ESI m/z: 814 (M+H)⁺) was obtained as a white solid after purification by reversed phase flash chromatography (0-100% acetonitrile in aq. TFA (0.01%)). To a suspension of Boc-P22 in DCM (4.5 mL) was added TFA (0.5 mL). After the suspension turned clear, the reaction solution was stirred at room temperature for an hour until Boc was totally removed according to LCMS. The resulting mixture was concentrated in vacuo and the residue was purified by reversed phase flash chromatography (0-100% acetontrile in aq. ammonium bicarbonate (10 mM)) to give P22 (10 mg, 50% yield) as a white solid. ESI m/z: 814 (M+H)⁺. ¹H NMR (400 MHz, DMSO_{d6}) δ 9.26 (s, 1H), 8.18 (s, 1H), 8.12 (d, J=10.0 Hz, 1H), 7.74 (br s, 1H), 6.94 (d, J=8.4 Hz, 2H), 6.63 (d, J=8.4 Hz, 2H), 5.65 (d, J=13.2 Hz, 1H), 4.40 (t, J=9.6 Hz, 1H), 4.22 (br s, 1H), 3.67-3.60 (m, 1H), 3.05-2.89 (m, 2H), 2.72-2.59 (m, 2H), 2.33-2.23 (m, 1H), 2.14 (br s, 4H), 1.98-1.91 (m, 1H), 1.92-1.80 (m, 3H), 1.76-1.40 (m, 8H), 1.26 (br s, 10H), 1.06-0.99 (m, 7H), 0.96 (d, J=6.4 Hz, 3H), 0.88-0.81 (m, 10H), 0.65 (d, J=5.6 Hz, 3H) ppm.

### P23: (4S)-4-({2-[(1R,3R)-3-[(2S,3S)—N-hexyl-3-methyl-2-{[(2R)-2-methylpyrrolidin-2-yl]formamido}pentanamido]-4-methyl-1-[(methylcarbamoyl)oxy]pentyl]-1,3-thiazol-4-yl}formamido)-5-(4-hydroxyphenyl)-2,2-dimethylpentanoic Acid (P23)

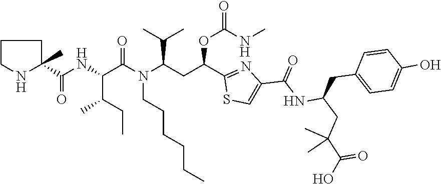 [SMILES: CCCCCCN(C(=O)[C@@H](NC(=O)[C@@]1(C)CCCN1)[C@@H](C)CC)[C@H](C[C@@H](OC(=O)NC)c1nc(C(=O)N[C@@H](Cc2ccc(O)cc2)CC(C)(C)C(=O)O)cs1)C(C)C]

Following General Procedure VI for payloads from compound 3Ed with compound TUPd, Boc-P23 (25 mg) was obtained as a white solid. Boc-P23 was then suspended in DCM (3.6 mL). To the suspension was added TFA (0.4 mL), and the mixture turned clear. The reaction solution was stirred at room temperature for an hour, and monitored by LCMS. The resulting mixture was concentrated in vacuo and the crude product was purified by prep-HPLC (0-100% acetonitrile in aq. ammonium bicarbonate (10 mM)) to give P23 (15 mg, 22% yield from 3Ed) as a white solid. ESI m/z: 829 (M+H)⁺. ¹H NMR (400 MHz, DMSO_{d6}) δ 9.23 (s, 1H), 8.16-8.12 (m, 2H), 7.43-7.42 (m, 2H), 6.93 (d, J=8.4 Hz, 2H), 6.63 (d, J=8.4 Hz, 2H), 5.58-5.54 (m, 1H), 4.40 (t, J=9.6 Hz, 1H), 4.25 (br s, 1H), 3.58 (br s, 1H), 3.04-2.91 (m, 2H), 2.73-2.61 (m, 3H), 2.57 (d, J=4.4 Hz, 3H), 2.17-1.96 (m, 3H), 1.90-1.72 (m, 4H), 1.66-1.42 (m, 6H), 1.26 (br s, 10H), 1.05 (br s, 7H), 0.95 (d, J=6.4 Hz, 3H), 0.88-0.81 (m, 9H), 0.67 (br s, 3H) ppm.

TABLE 3-1
Compound List of Tubulysin Modified on Substituted-Tup-Aniline

| | | | | | | HPLC | HPLC |
|---|---|---|---|---|---|---|---|
| | | | | | Mass | purity | RT |
| # | Structures | cLogP | MF | MW | m/z | (%) | (min) |
| P24 | 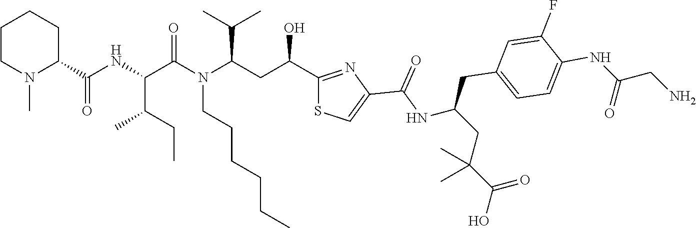 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(NC(=O)CN)c(F)c2)CC(C)(C)C(=O)O)cs1)C(C)C] | 3.23 | C₄₄H₇₀FN₇O₇S | 860.1 | 861 (M + H) | 95 | 7.68 (B) |
| P25 | 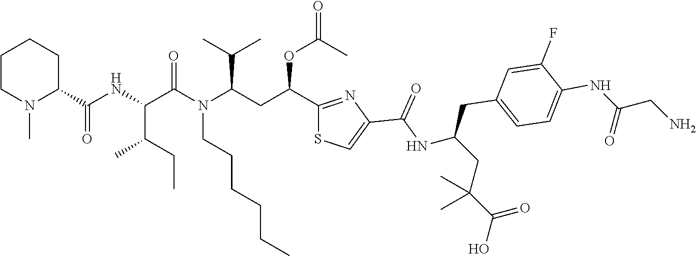 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] | 3.68 | C₄₆H₇₂FN₇O₈S | 902.2 | 452 (M/2 + H) | 98 | 6.27 (A) |
| P26 | 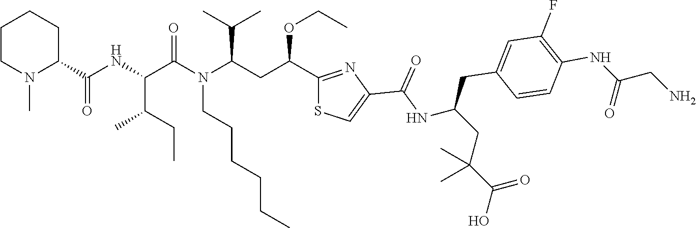 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)c(F)c2)CC(C)(C)C(=O)O)cs1)C(C)C] | 4.23 | C₄₆H₇₄FN₇O₇S | 888.2 | 889 (M + H) | 99 | 8.26 (B) |
| P27 | 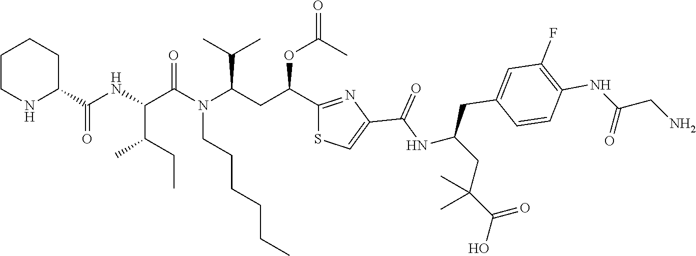 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] | 3.16 | C₄₅H₇₀FN₇O₈S | 888.2 | 889 (M + H) | 98 | 5.91 (A) |
| P28 | 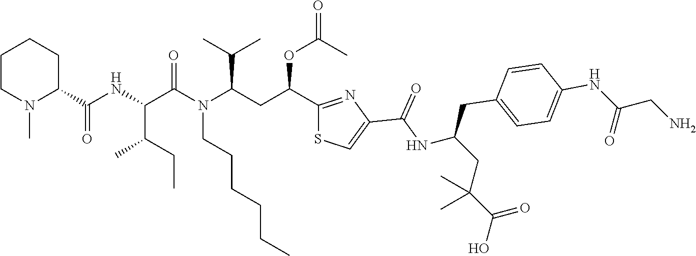 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] | 3.53 | C₄₆H₇₃N₇O₈S | 884.2 | 443 (M/2 + H) | 95 | 7.99 (B) |
| P29 | 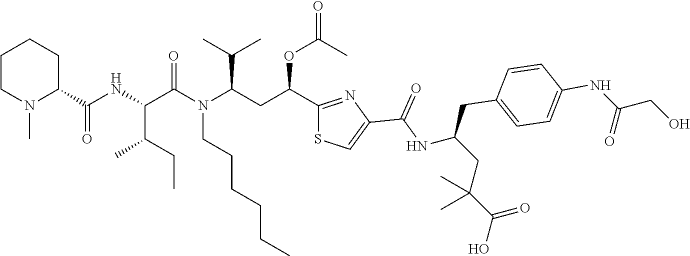 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CO)cc2)CC(C)(C)C(=O)O)cs1)C(C)C] | 3.57 | C₄₆H₇₂N₆O₉S | 885.2 | 885 (M + H) | 98 | 8.06 (B) |
| P30 | 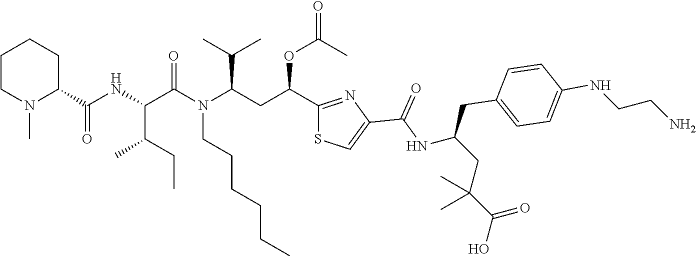 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCCN)cc2)CC(C)(C)C(=O)O)cs1)C(C)C] | 4.15 | C₄₆H₇₅N₇O₇S | 870.2 | 436 (M/2 + H) | 96 | 5.76 (A) |
| P31 | 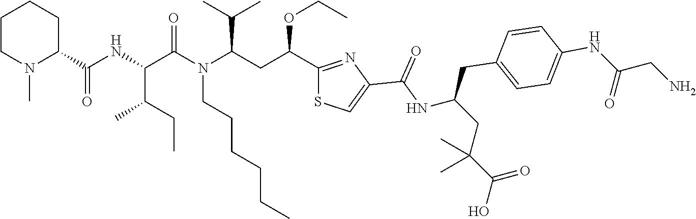 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] | 4.09 | C₄₆H₇₅N₇O₇S | 870.2 | 436 (M/2 + H) | 99 | 8.42 (B) |
| P32 | 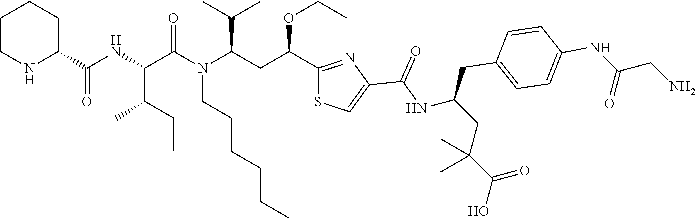 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] | 3.58 | C₄₅H₇₃N₇O₇S | 856.2 | 857 (M + H); 429 (M/2 + H) | 99 | 8.66 (B) |

TABLE 3-2
Modification on Substituted-Tup-Aniline

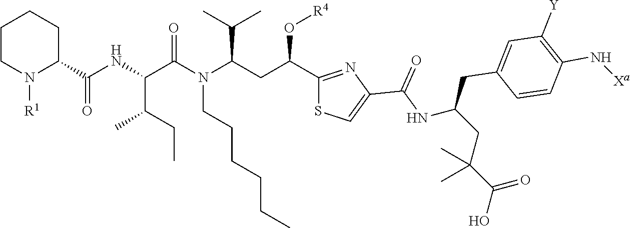

| | | | | | | | | HCT-15 with | | SK-BR-3 | |
|---|---|---|---|---|---|---|---|---|---|---|---|
| | | | | | | HCT-15 | | verapamil | | | Ratio |
| | Structures | | | | | IC₅₀ | Ratio | IC₅₀ | Ratio | IC₅₀ | to Xa = |
| # | R¹ | R⁴ | X^{a} | Y | cLogP | (nM) | to Xa = H | (nM) | to Xa = H | (nM) | H |
| P24 | Me | H | COCH₂NH₂ | F | 3.23 | 15.5 | 5.2x | 0.82 | 3.2x | 0.86 | 22x |
| P25 | Me | Ac | COCH₂NH₂ | F | 3.68 | 0.08 | 4.0x | 0.04 | 4.0x | 0.03 | 1.9x |
| P26 | Me | Et | COCH₂NH₂ | F | 4.23 | 0.25 | 4.9x | 0.05 | 2.8x | 0.05 | 2.6x |
| P27 | H | Ac | COCH₂NH₂ | F | 3.16 | 1.48 | 6.4x | 0.09 | 3.8x | 0.11 | 3.9x |
| P28 | Me | Ac | COCH₂NH₂ | H | 3.53 | 0.17 | 6.9x | 0.11 | 5.8x | 0.03 | 2.5x |
| P29 | Me | Ac | COCH₂OH | H | 3.57 | 4.68 | 104x | 0.67 | 35x | | |
| P30 | Me | Ac | CH₂CH₂NH₂ | H | 4.15 | 2.64 | 155x | 0.42 | 22x | 0.45 | 34x |
| P31 | Me | Et | COCH₂NH₂ | H | 4.09 | 0.81 | 9.7x | 0.29 | 4.3x | 0.07 | 2.4x |
| P32 | H | Et | COCH₂NH₂ | H | 3.58 | >100 | | >100 | | >1 | |

Synthesis of Tubulysin Payloads in Table 3

### P24: (4S)-5-[4-(2-aminoacetamido)-3-fluorophenyl]-4-({2-[(1R,3R)-3-[(2S,3S)—N-hexyl-3-methyl-2-{[(2R)-1-methylpiperidin-2-yl]formamido}pentanamido]-1-hydroxy-4-methylpentyl]-1,3-thiazol-4-yl}formamido)-2,2-dimethylpentanoic Acid (P24)

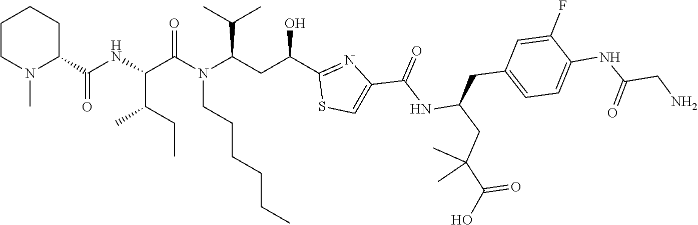 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(NC(=O)CN)c(F)c2)CC(C)(C)C(=O)O)cs1)C(C)C]

Following General Procedure VI from compound 3 Da (45 mg, 79 μmol) with intermediate TUPf, Fmoc-P24 (45 mg, ESI m/z: 542 (M/2+H)⁺) was obtained as a white solid after purification by reversed phase flash chromatography (0-100% acetonitrile in aq. TFA (0.01%)). To a solution of Fmoc-P24 (45 mg) in DMF (3 mL) was added piperidine (14 mg, 0.17 mmol), and the reaction mixture was stirred at room temperature for 3 hours until Fmoc was totally removed according to LCMS. The resulting mixture was directly purified by reversed phase flash chromatography (0-50% acetonitrile in aq. formic acid (0.01%)) to give P24 (10 mg, 15% yield from 3 Da) as a white solid. ESI m/z: 861 (M+H)⁺, 431 (M/2+H)⁺. ¹H NMR (400 MHz, DMSO_{d6}) δ 8.12 (s, 1H), 8.04 (t, J=8.4 Hz, 1H), 7.84-7.72 (m, 1H), 7.07 (d, J=12.4 Hz, 1H), 6.96 (d, J=8.4 Hz, 1H), 6.30 (s, 1H), 4.54-4.44 (m, 2H), 4.14 (s, 1H), 3.73 (t, J=10.8 Hz, 1H), 3.26 (s, 2H), 3.03 (s, 1H), 2.84-2.78 (m, 2H), 2.76-2.71 (m, 1H), 2.02 (s, 3H), 1.94-1.90 (m, 2H), 1.87-1.79 (m, 3H), 1.58-1.52 (m, 3H), 1.49-1.44 (m, 2H), 1.30-1.22 (m, 8H), 1.20-1.16 (m, 1H), 1.16-1.08 (m, 3H), 1.01-0.95 (m, 7H), 0.91 (d, J=6.4 Hz, 3H), 0.88-0.79 (m, 12H), 0.74 (s, 3H) ppm. ¹⁹F NMR (376 MHz, DMSO_{d6}) 6-129.7 ppm.

### P25: (4S)-4-({2-[(1R,3R)-1-(acetyloxy)-3-[(2S,3S)—N-hexyl-3-methyl-2-{[(2R)-1-methylpiperidin-2-yl]formamido}pentanamido]-4-methylpentyl]-1,3-thiazol-4-yl}formamido)-5-[4-(2-aminoacetamido)-3-fluorophenyl]-2,2-dimethylpentanoic Acid (P25)

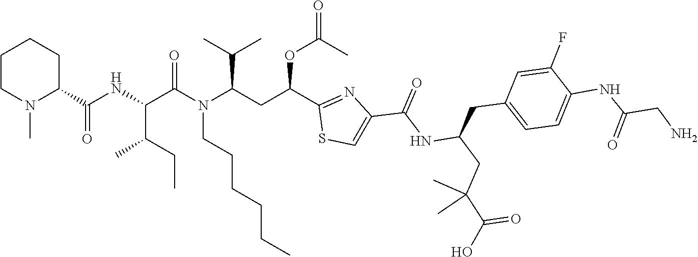 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C]

Following General Procedure VI from compound 3Fa (23 mg, 38 μmol) with intermediate TUPf, Fmoc-P25 (40 mg, ESI m/z: 1124 (M+H)⁺) was obtained as a white solid after purification by reversed phase flash chromatography (0-100% acetonitrile in aq. TFA (0.01%)). To a solution of Fmoc-P25 (40 mg) in DMF (4 mL) was added diethylamine (1 mL), and the reaction mixture was stirred at room temperature for an hour until Fmoc was totally removed according to LCMS. The resulting mixture was directly purified by prep-HPLC (0-100% acetonitrile in aq. TFA (0.01%)) to give P25 (15 mg, 39% yield from 3Fa, TFA salt) as a white solid. ESI m/z: 452 (M/2+H)⁺. ¹H NMR (400 MHz, DMSO_{d6}) δ 10.15 (s, 1H), 9.72 (s, 1H), 9.12 (d, J=9.3 Hz, 1H), 8.16 (s, 1H), 8.08 (s, 2H), 7.93-7.82 (m, 1H), 7.78 (t, J=8.3 Hz, 1H), 7.24 (s, 1H), 7.14-7.08 (m, 1H), 7.05-6.96 (m, 1H), 5.68-5.59 (m, 1H), 4.52 (t, J=9.0 Hz, 1H), 4.31-4.22 (m, 1H), 3.81 (s, 2H), 3.68-3.55 (m, 1H), 3.11-3.03 (m, 2H), 2.97-2.89 (m, 1H), 2.83-2.71 (m, 2H), 2.69-2.60 (m, 3H), 2.35-2.25 (m, 2H), 2.13 (s, 3H), 2.02-1.90 (m, 3H), 1.83-1.72 (m, 3H), 1.63-1.54 (m, 2H), 1.49-1.21 (m, 11H), 1.16 (t, J=7.3 Hz, 2H), 1.09 (s, 3H), 1.07 (s, 3H), 0.96 (d, J=6.4 Hz, 3H), 0.91-0.78 (m, 9H), 0.71 (d, J=6.1 Hz, 3H) ppm. ¹⁹F NMR (376 MHz, DMSO_{d6}) 6-73.5, −125.6 ppm. >99.9% ee using AD, AS, OD, and OJ columns.

### P26: (4S)-5-[4-(2-aminoacetamido)-3-fluorophenyl]-4-({2-[(1R,3R)-1-ethoxy-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic Acid (P26)

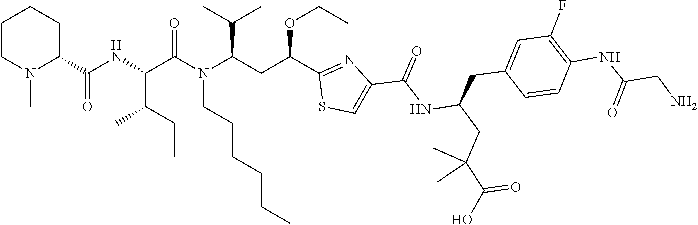 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)c(F)c2)CC(C)(C)C(=O)O)cs1)C(C)C]

Following General Procedure VI from compound 3Ba (50 mg, 84 μmol) with intermediate TUPf, Fmoc-P26 (30 mg, ESI m/z: 556 (M/2+H)⁺) was obtained as a white solid after purification by reversed phase flash chromatography (0-100% acetonitrile in aq. TFA (0.01%)). To a solution of Fmoc-P26 (65 mg) in DMF (4 mL) was added piperidine (20 μL), and the reaction mixture was stirred at room temperature for an hour until Fmoc was totally removed according to LCMS. The resulting mixture was directly purified by prep-HPLC (0-100% acetonitrile in aq. ammonium bicarbonate (10 mM)) to give P26 (10 mg, 13% yield from 3Ba) as a white solid. ESI m/z: 889 (M+H)⁺, 445 (M/2+H)⁺. ¹H NMR (400 MHz, DMSO_{d6}) δ 8.14 (s, 1H), 8.04 (t, J=8.3 Hz, 1H), 7.72 (s, 1H), 7.54 (s, 1H), 7.06 (d, J=11.0 Hz, 1H), 6.95 (d, J=8.4 Hz, 1H), 4.59-4.43 (m, 1H), 4.32-4.27 (m, 2H), 3.76-3.72 (m, 1H), 3.59-3.50 (m, 2H), 2.97-2.84 (m, 3H), 2.76 (d, J=6.0 Hz, 2H), 2.09 (s, 3H), 1.97-1.79 (m, 7H), 1.74-1.69 (m, 1H), 1.68-1.35 (m, 8H), 1.25-1.23 (m, 7H), 1.17 (t, J=7.0 Hz, 4H), 1.09 (s, 3H), 1.07 (s, 3H), 1.06-0.82 (m, 14H), 0.70 (s, 3H) ppm. ¹⁹F NMR (376 MHz, DMSO_{d6}) δ −129.5 ppm.

### P27: (4S)-4-({2-[(1R,3R)-1-(acetyloxy)-3-[(2S,3S)—N-hexyl-3-methyl-2-[(2R)-piperidin-2-ylformamido]pentanamido]-4-methylpentyl]-1,3-thiazol-4-yl}formamido)-5-[4-(2-aminoacetamido)-3-fluorophenyl]-2,2-dimethylpentanoic Acid (P27)

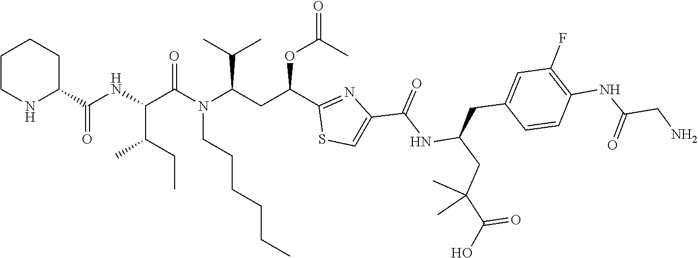 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C]

Following General Procedure VI from compound 3Fb (46 mg, 66 μmol) with intermediate TUPf, Fmoc-Boc-P27 (65 mg, ESI m/z: 1111 (M−Boc+H)⁺, 1233 (M+Na)⁺) was obtained as a white solid after purification by reversed phase flash chromatography (0-100% acetonitrile in aq. TFA (0.01%)). To a solution of Fmoc-Boc-P27 (65 mg) in DCM (6 mL) was added TFA (2 mL), and the reaction mixture was stirred at room temperature for 3 hours, and monitored by LCMS. The volatiles were removed in vacuo to give crude Fmoc-P27 (ESI m/z: 1110 (M+H)⁺) as a white solid. Fmoc-P27 was dissolved in DMF (5 mL). To the solution was added diethylamine (1 mL) and the reaction mixture was stirred at room temperature for an hour, and monitored by LCMS. The resulting mixture was directly purified by prep-HPLC (0-100% acetonitrile in aq. TFA (0.01%)) to give P27 (12 mg, TFA salt, 20% yield from 3Fb) as a white solid. ESI m/z: 889 (M+H)⁺, 445 (M/2+H)⁺. ¹H NMR (400 MHz, DMSO_{d6}) δ 8.29 (s, 1H), 8.15 (s, 1H), 8.07-8.00 (m, 1H), 7.87-7.79 (m, 1H), 7.76-7.67 (m, 1H), 7.07 (dd, J=12.1 and 1.5 Hz, 1H), 6.97 (dd, J=8.8 and 0.6 Hz, 1H), 5.66 (d, J=13.0 Hz, 1H), 4.52-4.45 (m, 1H), 4.33-4.21 (m, 2H), 2.91-2.81 (m, 3H), 2.80-2.74 (m, 2H), 2.70-2.62 (m, 1H), 2.35-2.31 (m, 1H), 2.28-2.19 (m, 2H), 2.14 (s, 3H), 2.04-1.96 (m, 2H), 1.92-1.79 (m, 3H), 1.77-1.66 (m, 3H), 1.64-1.54 (m, 2H), 1.53-1.42 (m, 3H), 1.36-1.18 (m, 12H), 1.15-1.10 (m, 7H), 0.95 (d, J=6.5 Hz, 3H), 0.89-0.74 (m, 9H), 0.70 (d, J=6.7 Hz, 3H) ppm. ¹⁹F NMR (376 MHz, DMSO_{d6}) 6-73.41, −129.5 ppm.

### P28: (4S)-4-({2-[(1R,3R)-1-(acetyloxy)-3-[(2S,3S)—N-hexyl-3-methyl-2-{[(2R)-1-methylpiperidin-2-yl]formamido}pentanamido]-4-methylpentyl]-1,3-thiazol-4-yl}formamido)-5-[4-(2-aminoacetamido)phenyl]-2,2-dimethylpentanoic Acid (P28)

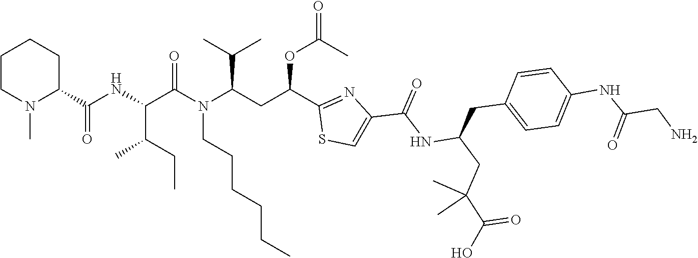 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C]

Following General Procedure VI from compound 3Fa with intermediate TUPg, Fmoc-P28 (26 mg, 23% yield, ESI m/z: 554 (M/2+H)⁺) was obtained as a white solid. Fmoc-P28 was dissolved in DMF (3 mL). To the solution was added piperidine (10 mg, 0.12 mmol), and the reaction mixture was stirred at room temperature for 3 hours until Fmoc was totally removed, according to LCMS. The resulting solution was directly purified by prep-HPLC (0-100% acetonitrile in aq. ammonium bicarbonate (10 mM)) to give payload P28 (12 mg, 11% yield from 3Fa) as a white solid. ESI m/z 443 (M/2+H)⁺. ¹H NMR (500 MHz, DMSO_{d6}) δ 8.16 (s, 1H), 7.66 (br s, 1H), 7.64 (br s, 1H), 7.51 (d, J=8.5 Hz, 2H), 7.20 (br s, 1H), 7.09 (d, J=8.5 Hz, 2H), 5.64 (d, J=13 Hz, 1H), 5.32 (t, J=5.0 Hz, 1H), 4.74 (t, J=8.5, 1H), 4.30-4.23 (m, 1H), 3.71-3.62 (m, 1H), 3.22 (s, 2H), 3.01-2.94 (m, 1H), 2.84-2.81 (m, 1H), 2.74-2.69 (m, 2H), 2.65-2.63 (m, 1H), 2.37-2.34 (m, 1H), 2.29-2.22 (m, 1H), 2.13 (s, 3H), 2.07 (s, 3H), 2.02-1.96 (m, 2H), 1.93-1.84 (m, 3H), 1.69-1.59 (m, 3H), 1.54-1.43 (m, 4H), 1.27-1.21 (m, 11H), 1.06 (s, 3H), 1.05 (s, 3H), 0.95 (d, J=6.0 Hz, 3H), 0.86-0.79 (m, 9H), 0.68 (d, J=6.0 Hz, 3H) ppm.

### P29: (4S)-4-({2-[(1R,3R)-1-(acetyloxy)-3-[(2S,3S)—N-hexyl-3-methyl-2-{[(2R)-1-methylpiperidin-2-yl]formamido}pentanamido]-4-methylpentyl]-1,3-thiazol-4-yl}formamido)-5-[4-(2-hydroxyacetamido)phenyl]-2,2-dimethylpentanoic Acid (P29)

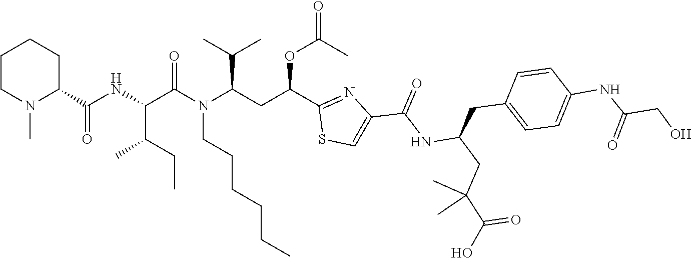 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CO)cc2)CC(C)(C)C(=O)O)cs1)C(C)C]

Following General Procedure VI from compound 3Fa with intermediate TUPk, P29 (22 mg, 25% yield) was obtained as a white solid. ESI m/z 885.3 (M+H)⁺. ¹H NMR (500 MHz, DMSO_{d6}) δ 12.10 (br s, 1H), 9.53 (s, 1H), 8.15 (s, 1H), 7.65 (br s, 1H), 7.62 (br s, 1H), 7.58 (d, J=8.5 Hz, 2H), 7.08 (d, J=8.5 Hz, 2H), 5.66-5.61 (m, 2H), 4.48 (t, J=10 Hz, 1H), 4.30-4.22 (m, 1H), 3.94 (d, J=5.0 Hz, 2H), 3.72-3.60 (m, 1H), 3.00-2.96 (m, 1H), 2.84-2.81 (m, 1H), 2.78-2.67 (m, 2H), 2.30-2.23 (m, 1H), 2.13 (s, 3H), 2.07 (s, 1H), 2.02-1.85 (m, 5H), 1.73-1.60 (m, 5H), 1.55-1.33 (m, 5H), 1.31-1.23 (m, 9H), 1.18-1.08 (m, 2H), 1.06 (s, 3H), 1.05 (s, 3H), 0.95 (d, J=6.5 Hz, 3H), 0.86-0.80 (m, 9H), 0.68 (d, J=6.5 Hz, 3H) ppm.

### P30: (4S)-4-({2-[(1R,3R)-1-(acetyloxy)-3-[(2S,3S)—N-hexyl-3-methyl-2-{[(2R)-1-methylpiperidin-2-yl]formamido}pentanamido]-4-methylpentyl]-1,3-thiazol-4-yl}formamido)-5-{4-[(2-aminoethyl)amino]phenyl}-2,2-dimethylpentanoic Acid (P30)

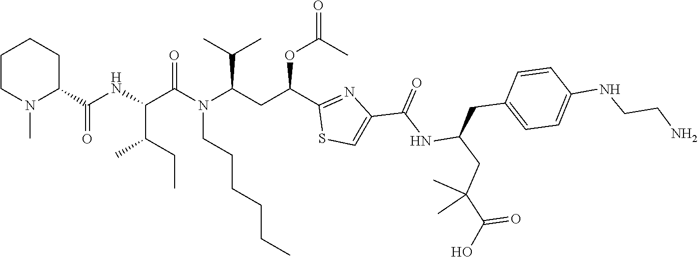 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCCN)cc2)CC(C)(C)C(=O)O)cs1)C(C)C]

Following General Procedure VI from compound 3Fa (42 mg, 69 μmol) with intermediate TUPl, Fmoc-P30 (52 mg, ESI m/z: 1094 (M+H)⁺) was obtained as a white solid. Fmoc-P30 was dissolved in DMF (1 mL). To the solution was added diethylamine (1 mL), and the reaction mixture was stirred at room temperature for an hour until Fmoc was totally removed according to LCMS. The reaction mixture was directly purified by prep-HPLC (0-100% acetonitrile in aq. TFA (0.03%)) to give P30 (33 mg, 49% yield from 3Fa, TFA salt) as a white solid. ESI m/z: 872 (M+H)⁺. ¹H NMR (400 MHz, DMSO_{d6}) δ 8.17 (s, 1H), 7.74 (d, J=8.9 Hz, 1H), 7.64 (d, J=8.5 Hz, 1H), 6.93 (d, J=8.4 Hz, 2H), 6.50 (d, J=8.4 Hz, 2H), 5.65 (d, J=12.8 Hz, 1H), 5.58 (s, 1H), 4.48 (t, J=9.3 Hz, 1H), 4.20 (s, 1H), 3.76-3.66 (m, 1H), 2.98-2.87 (m, 12H), 2.14 (s, 3H), 2.08 (s, 3H), 1.92-1.80 (m, 3H), 1.69-1.59 (m, 3H), 1.32-1.29 (m, 4H), 1.19-1.12 (m, 14H), 1.05 (d, J=4.9 Hz, 6H), 0.95 (d, J=6.4 Hz, 3H), 0.90-0.79 (m, 9H), 0.69 (d, J=5.6 Hz, 3H) ppm. ¹⁹F NMR (376 MHz, DMSO_{d6}) 6-73.56 ppm.

### P31: (4S)-5-[4-(2-aminoacetamido)phenyl]-4-({2-[(1R,3R)-1-ethoxy-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic Acid (P31)

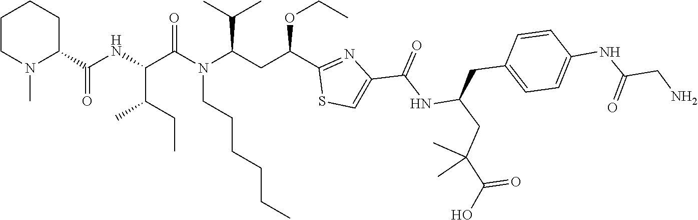 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C]

Following General Procedure VI from compound 3Ba with intermediate TUPg, Fmoc-P31 (27 mg, ESI m/z: 557 (M/2+H)⁺) was obtained as a white solid. Fmoc-P31 was dissolved in DMF (3 mL). To the solution was added piperidine (10 mg, 0.12 mmol), and the reaction mixture was stirred at room temperature for 3 hours until Fmoc was totally removed, according to LCMS. The resulting solution was purified directly by prep-HPLC (0-100% acetonitrile in aq. TFA (0.03%)) to give payload P31 (12 mg, 11% yield) as a white solid. ESI m/z 435.7 (M/2+H)⁺, 870.5 (M+H)⁺. ¹H NMR (400 MHz, DMSO_{d6}) δ 12.10 (s, 1H), 10.37 (s, 1H), 9.80-9.68 (br s, 1H), 9.13 (d, J=8.4 Hz, 1H), 8.18-8.11 (m, 3H), 7.72-7.56 (br s, 1H), 7.46 (d, J=8.4 Hz, 2H), 7.16 (d, J=8.0 Hz, 2H), 4.55 (t, J=8.8 Hz, 1H), 4.34-4.31 (m, 1H), 4.26-4.22 (m, 1H), 3.78-3.66 (m, 3H), 3.24-3.09 (m, 3H), 2.79-2.74 (m, 1H), 2.69-2.65 (m, 4H), 2.12-1.57 (m, 14H), 1.47-1.36 (m, 11H), 1.16 (t, J=6.8 Hz, 3H), 1.05 (d, J=8.4 Hz, 6H), 0.93-0.82 (m, 12H), 0.77-0.67 (m, 3H) ppm. ¹⁹F NMR (376 MHz, DMSO_{d6}) 6-73.5 ppm.

### P32: (4S)-5-[4-(2-aminoacetamido)phenyl]-4-({2-[(1R,3R)-1-ethoxy-3-[(2S,3S)—N-hexyl-3-methyl-2-[(2R)-piperidin-2-ylformamido]pentanamido]-4-methylpentyl]-1,3-thiazol-4-yl}formamido)-2,2-dimethylpentanoic Acid (P32)

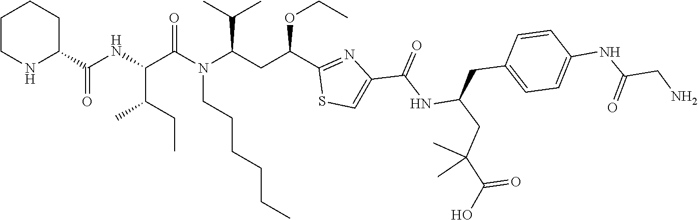 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C]

Following General Procedure VI from compound 3Bb with intermediate TUPg, Fmoc-Boc-P32 (50 mg, crude, ESI m/z: 1178.5 (M+H)⁺) was obtained as yellow oil. Fmoc-Boc-P32 was dissolved in DCM (4 mL). To the solution was added TFA (1 mL), and the reaction solution was stirred at room temperature for an hour until Boc was totally removed, according to LCMS. The resulting mixture was concentrated in vacuo and the residue (ESI m/z: 1079 (M+H)⁺) was dissolved in DCM (4 mL). To the solution was added piperidine (20 L), and the mixture was stirred at room temperature for an hour until Fmoc was removed in vacuo, according to LCMS. The resulting mixture was concentrated in vacuo and the residue was purified by prep-HPLC (0-100% acetonitrile in aq. TFA (0.03%)) to give P32 (10 mg, 18% yield from 3Bb, dual-TFA salt) as a white solid. ESI m/z: 857 (M+H)⁺, 429 (M/2+H)⁺. ¹H NMR (400 MHz, DMSO_{d6}) δ 12.01 (s, 1H), 10.35 (s, 1H), 8.83 (d, J=9.3 Hz, 1H), 8.42 (s, 1H), 8.35-8.06 (m, 4H), 7.62 (s, 1H), 7.46 (d, J=8.5 Hz, 2H), 7.15 (d, J=8.4 Hz, 2H), 4.60 (t, J=14 Hz, 1H), 4.50-4.25 (m, 2H), 3.74 (s, 3H), 3.68-3.43 (m, 3H), 3.22-3.10 (m, 1H), 3.09-2.90 (m, 2H), 2.77-2.63 (m, 2H), 2.17-2.02 (m, 1H), 2.02-1.37 (m, 23H), 1.17 (t, J=7.0 Hz, 3H), 1.04 (d, J=8.8 Hz, 6H), 0.93-0.75 (m, 12H), 0.74 (d, J=6.1 Hz, 3H) ppm.

TABLE 4-1
Compound List of N—O Tubulysin Payloads

| | | | | | | HPLC | HPLC |
|---|---|---|---|---|---|---|---|
| | | | | | Mass | purity | RT |
| # | Structures | cLogP | MF | MW | m/z | (%) | (min) |
| P33 | 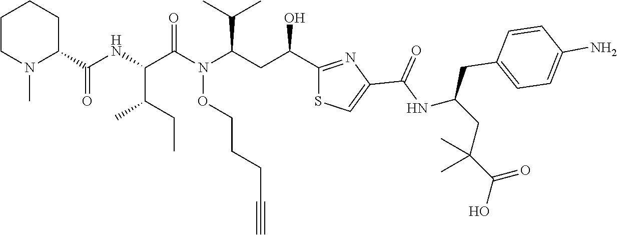 [SMILES: C#CCCCON(C(=O)[C@@H](NC(=O)[C@H]1CCCCN1C)[C@@H](C)CC)[C@H](C[C@@H](O)c1nc(C(=O)N[C@@H](Cc2ccc(N)cc2)CC(C)(C)C(=O)O)cs1)C(C)C] | 2.80 | C₄₁H₆₂N₆O₇S | 783.0 | 783 (M + H) | >99 | 7.37 (B) |
| P34 | 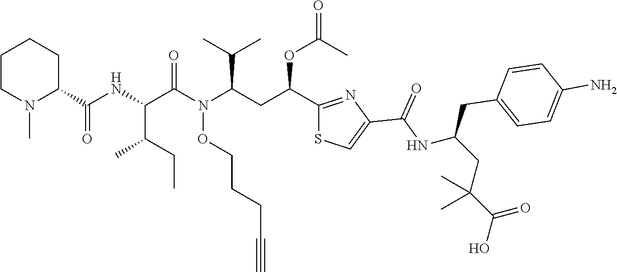 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] | 3.24 | C₄₃H₆₄N₆O₈S | 825.1 | 413 (M/2 + H) | 95 | 6.98 (A) |
| P35 | 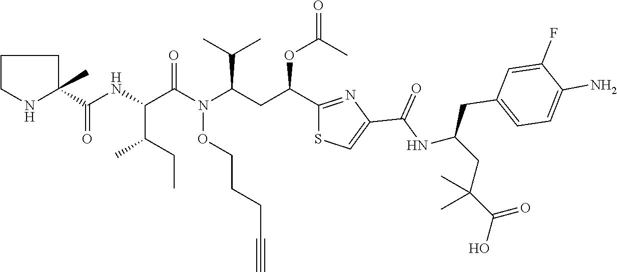 [SMILES: C#CCCCON(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] | 3.14 | C₄₂H₆₁FN₆O₈S | 829.0 | 415 (M/2 + H) | 99 | 7.80 (B) |
| P36 | 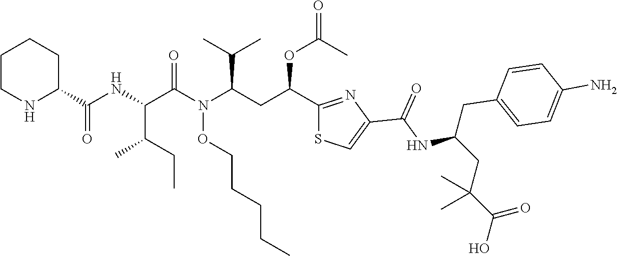 [SMILES: CCCCCON(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] | 3.81 | C₄₂H₆₆N₆O₈S | 815.1 | 408 (M/2 + H) | 99 | 6.35 (A) |
| P51 | 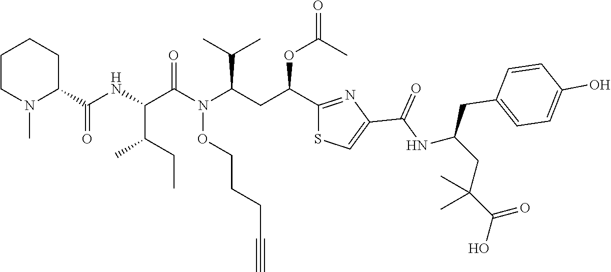 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] | 3.93 | C₄₃H₆₃N₅O₉S | 826.1 | 826 (M + H) | 95 | 6.50 (A) |

TABLE 4-2
Cytotoxicity of Tubulysin Payloads in Table 4

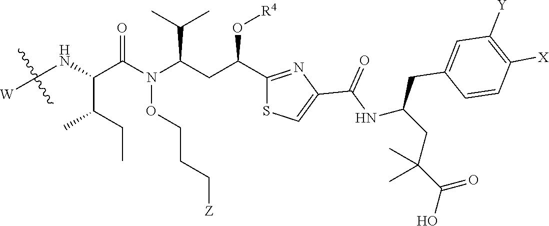

| | Structures | | | | | | HCT-15 IC₅₀ | HCT-15 with |
|---|---|---|---|---|---|---|---|---|
| # | W | R⁴ | Z | X | Y | cLogP | (nM) | verapamil IC₅₀ (nM) |
| P33 | 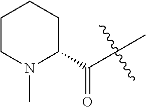 [SMILES: *C(=O)[C@H]1CCCCN1C] | H | C≡CH | NH₂ | H | 2.80 | 81.7 | 28.1 |
| P34 | 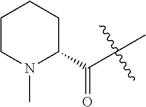 [SMILES: *C(=O)[C@H]1CCCCN1C] | Ac | C≡CH | NH₂ | H | 3.24 | 0.41 | 0.24 |
| P35 | 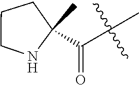 [SMILES: *C(=O)[C@@]1(C)CCCN1] | Ac | C≡CH | NH₂ | F | 3.14 | 4.87 | |
| P36 | 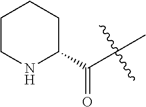 [SMILES: *C(=O)[C@H]1CCCCN1] | Ac | Et | NH₂ | H | 3.81 | 12.2 | |
| P51 | 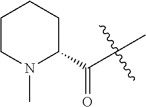 [SMILES: *C(=O)[C@H]1CCCCN1C] | Ac | C≡CH | OH | H | 3.93 | 0.41 | 0.16 |

Synthesis of Intermediate 2G

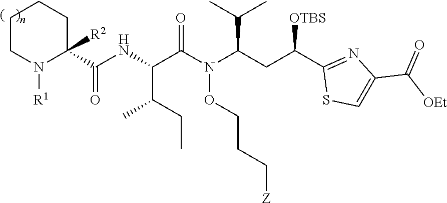

### Ethyl 2-[(1R,3R)-1-[(tert-butyldimethylsilyl)oxy]-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazole-4-carboxylate (2Ga)

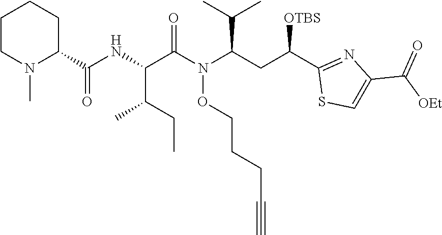 [SMILES: C#CCCCON(C(=O)[C@@H](NC(=O)[C@H]1CCCCN1C)[C@@H](C)CC)[C@H](C[C@@H](O[Si](C)(C)C(C)(C)C)c1nc(C(=O)OCC)cs1)C(C)C]

Following General Procedure I from intermediate 1G (1.8 g, 3.1 mmol) with MEPa, compound 2Ga (1.7 g, 78% yield) was obtained as a viscous oil. ESI m/z: 707 (M+H)⁺. ¹H NMR (500 MHz, methanol_{d4}) δ 8.38 (s, 1H), 5.03 (d, J=8.1 Hz, 1H), 4.85 (t, J=6.3 Hz, 1H), 4.40 (q, J=7.5 Hz, 3H), 4.13-4.07 (m, 1H), 4.03-3.86 (m, 2H), 3.52-3.45 (m, 1H), 3.35-3.29 (m, 1H), 2.87 (s, 3H), 2.51-2.35 (m, 3H), 2.25 (t, J=2.4 Hz, 1H), 2.23-2.15 (m, 1H), 2.10-1.53 (m, 11H), 1.40 (t, J=7.1 Hz, 3H), 1.27-1.19 (m, 1H), 1.08-0.94 (m, 12H), 0.94 (s, 9H), 0.13 (s, 3H), −0.16 (s, 3H) ppm.

### Ethyl 2-[(1R,3R)-3-[(2S,3S)-2-{[(2R)-1-[(tert-butoxy)carbonyl]-2-methylpyrrolidin-2-yl]formamido}-3-methyl-N-(pent-4-yn-1-yloxy)pentanamido]-1-[(tert-butyldimethylsilyl)oxy]-4-methylpentyl]-1,3-thiazole-4-carboxylate (2Gb)

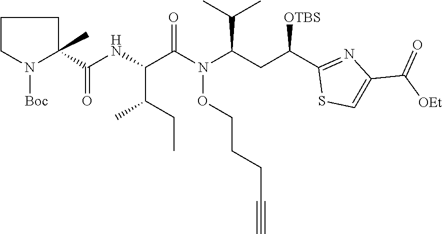 [SMILES: C#CCCCON(C(=O)[C@@H](NC(=O)[C@@]1(C)CCCN1C(=O)OC(C)(C)C)[C@@H](C)CC)[C@H](C[C@@H](O[Si](C)(C)C(C)(C)C)c1nc(C(=O)OCC)cs1)C(C)C]

Following General Procedure I from intermediate 1G (0.14 g, 0.24 mmol) with acid MEPf (56 mg, 0.24 mmol), compound 2Gb (0.15 g, 80% yield) was obtained as a yellow oil after purification by silica gel column chromatography (0-20% ethyl acetate in petroleum ether). ESI m/z: 793 (M+H)⁺.

### tert-Butyl (2R)-2-{[(1S,2S)-1-{[(1R,3R)-1-[(tert-butyldimethylsilyl)oxy]-1-[4-(ethoxycarbonyl)-1,3-thiazol-2-yl]-4-methylpentan-3-yl](pent-4-yn-1-yloxy)carbamoyl}-2-methylbutyl]carbamoyl}piperidine-1-carboxylate (2Gc′)

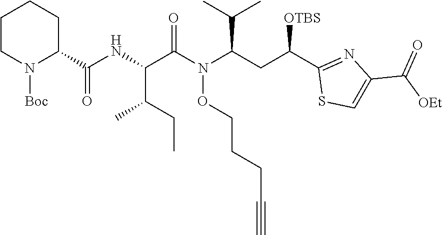 [SMILES: C#CCCCON(C(=O)[C@@H](NC(=O)[C@H]1CCCCN1C(=O)OC(C)(C)C)[C@@H](C)CC)[C@H](C[C@@H](O[Si](C)(C)C(C)(C)C)c1nc(C(=O)OCC)cs1)C(C)C]

Following General Procedure I from intermediate 1G (0.11 g, 0.19 mmol) with acid MEPb (43 mg, 0.19 mmol), compound 2Gc′ (0.11 g, 78% yield) was obtained as a white solid, and used in the next step without further purification. ESI m/z: 793 (M+H)⁺.

### tert-Butyl (2R)-2-{[(1S,2S)-1-{[(1R,3R)-1-[(tert-butyldimethylsilyl)oxy]-1-[4-(ethoxycarbonyl)-1,3-thiazol-2-yl]-4-methylpentan-3-yl](pentyloxy)carbamoyl}-2-methylbutyl]carbamoyl}piperidine-1-carboxylate (2Gc)

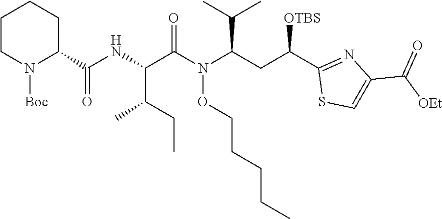 [SMILES: CCCCCON(C(=O)[C@@H](NC(=O)[C@H]1CCCCN1C(=O)OC(C)(C)C)[C@@H](C)CC)[C@H](C[C@@H](O[Si](C)(C)C(C)(C)C)c1nc(C(=O)OCC)cs1)C(C)C]

To a solution of compound 2Gc′ (0.11 g, 0.14 mmol) in ethyl acetate (10 mL) was added wet palladium on carbon (10% Pd, 11 mg, 10 wt %) under nitrogen. The mixture was degassed, purged with hydrogen 3 times, stirred under a hydrogen balloon at room temperature for 30 minutes, and monitored by LCMS. The resulting suspension was filtered through Celite and the filtrate was concentrated in vacuo to give crude compound 2Gc (0.11 g, crude) as a white solid. Crude 2Gc was used in the next step without further purification. ESI m/z: 797 (M+H)⁺.

Synthesis of Intermediate 2H

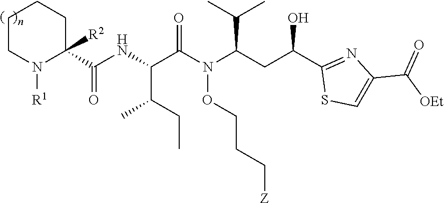

### Ethyl 2-[(1R,3R)-1-hydroxy-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazole-4-carboxylate (2Ha)

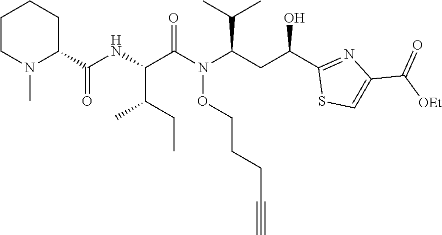 [SMILES: C#CCCCON(C(=O)[C@@H](NC(=O)[C@H]1CCCCN1C)[C@@H](C)CC)[C@H](C[C@@H](O)c1nc(C(=O)OCC)cs1)C(C)C]

Following General Procedure II from compound 2Ga, compound 2Ha (43 mg, 86% yield) was obtained as a white solid. ESI m/z: 593 (M+H)⁺.

### Ethyl 2-[(1R,3R)-3-[(2S,3S)-2-{[(2R)-1-[(tert-butoxy)carbonyl]-2-methylpyrrolidin-2-yl]formamido}-3-methyl-N-(pent-4-yn-1-yloxy)pentanamido]-1-hydroxy-4-methylpentyl]-1,3-thiazole-4-carboxylate (2Hb)

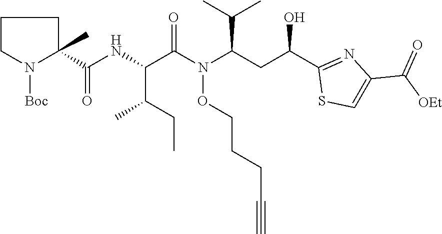 [SMILES: C#CCCCON(C(=O)[C@@H](NC(=O)[C@@]1(C)CCCN1C(=O)OC(C)(C)C)[C@@H](C)CC)[C@H](C[C@@H](O)c1nc(C(=O)OCC)cs1)C(C)C]

Following General Procedure II from 2Gb (0.13 g, 0.16 mmol), compound 2Hb (95 mg, 88% yield) was obtained as a yellow oil after purification by silica gel column chromatography (0-20% ethyl acetate in petroleum ether). ESI m/z: 679 (M+H)⁺, 701 (M+Na)⁺.

### tert-Butyl (2R)-2-{[(1S,2S)-1-{[(1R,3R)-1-[4-(ethoxycarbonyl)-1,3-thiazol-2-yl]-1-hydroxy-4-methylpentan-3-yl](pentyloxy)carbamoyl}-2-methylbutyl]carbamoyl}piperidine-1-carboxylate (2Hc)

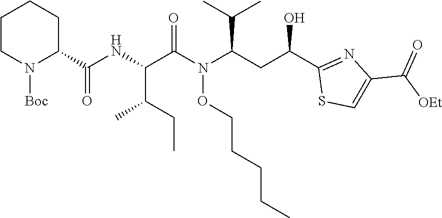 [SMILES: CCCCCON(C(=O)[C@@H](NC(=O)[C@H]1CCCCN1C(=O)OC(C)(C)C)[C@@H](C)CC)[C@H](C[C@@H](O)c1nc(C(=O)OCC)cs1)C(C)C]

Following General Procedure II from crude compound 2Gc (0.11 g), compound 2Hc (96 mg, 74% yield in 3 steps from intermediate 1G) was obtained as a white solid after purification by reversed phase flash chromatography (0-60% acetonitrile in aq. ammonium bicarbonate (10 mM)). ESI m/z: 683 (M+H)⁺.

Synthesis of Intermediate 3H

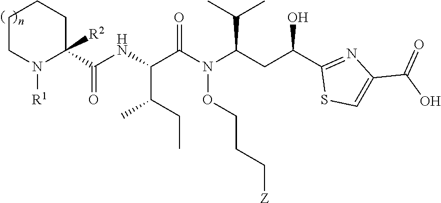

### 2-[(1R,3R)-1-Hydroxy-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazole-4-carboxylic Acid (3Ha)

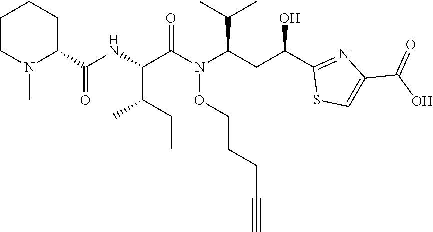 [SMILES: C#CCCCON(C(=O)[C@@H](NC(=O)[C@H]1CCCCN1C)[C@@H](C)CC)[C@H](C[C@@H](O)c1nc(C(=O)O)cs1)C(C)C]

Following General Procedure IV from 2Ha, compound 3Ha (37 mg, 90% yield) was obtained as a white solid. ESI m/z: 565 (M+H)⁺.

### 2-[(1R,3R)-3-[(2S,3S)-2-{[(2R)-1-[(tert-Butoxy)carbonyl]-2-methylpyrrolidin-2-yl]formamido}-3-methyl-N-(pent-4-yn-1-yloxy)pentanamido]-1-hydroxy-4-methylpentyl]-1,3-thiazole-4-carboxylic Acid (3Hb)

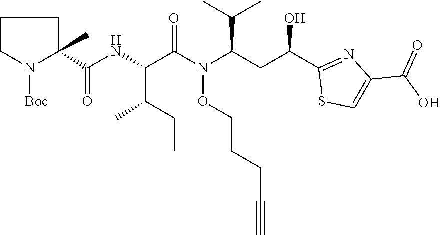 [SMILES: C#CCCCON(C(=O)[C@@H](NC(=O)[C@@]1(C)CCCN1C(=O)OC(C)(C)C)[C@@H](C)CC)[C@H](C[C@@H](O)c1nc(C(=O)O)cs1)C(C)C]

Following General Procedure IV from 2Hb (80 mg, 0.11 mmol), crude compound 3Hb (70 mg, 90% crude yield) was obtained as a yellow oil. ESI m/z: 673 (M+Na)⁺, 551.3 (M−Boc+H)⁺.

### 2-[(1R,3R)-3-[(2S,3S)-2-{[(2R)-1-[(tert-Butoxy)carbonyl]piperidin-2-yl]formamido}-3-methyl-N-(pentyloxy)pentanamido]-1-hydroxy-4-methylpentyl]-1,3-thiazole-4-carboxylic Acid (3Hc)

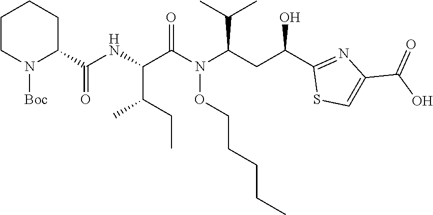 [SMILES: CCCCCON(C(=O)[C@@H](NC(=O)[C@H]1CCCCN1C(=O)OC(C)(C)C)[C@@H](C)CC)[C@H](C[C@@H](O)c1nc(C(=O)O)cs1)C(C)C]

Following General Procedure IV from compound 2Hc (96 mg, 0.14 mmol), compound 3Hc (69 mg, crude) was obtained as a white solid, and was used in the next step without purification. ESI m/z: 677 (M+Na)⁺.

Synthesis of Intermediate 31

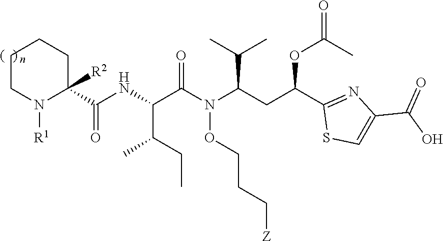

### 2-[(1R,3R)-1-(Acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazole-4-carboxylic Acid (3Ia)

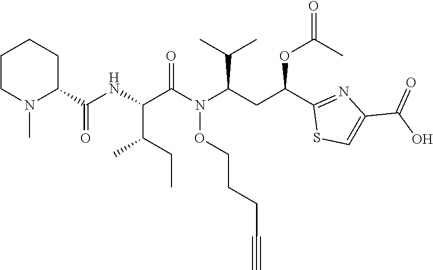 [SMILES: C#CCCCON(C(=O)[C@@H](NC(=O)[C@H]1CCCCN1C)[C@@H](C)CC)[C@H](C[C@@H](OC(C)=O)c1nc(C(=O)O)cs1)C(C)C]

Following General Procedure V from 3Ha, compound 3Ia (18 mg, 93% yield) was obtained as a white solid. ESI m/z: 607 (M+H)⁺.

### 2-[(1R,3R)-1-(Acetyloxy)-3-[(2S,3S)-2-{[(2R)-1-[(tert-butoxy)carbonyl]-2-methylpyrrolidin-2-yl]formamido}-3-methyl-N-(pent-4-yn-1-yloxy)pentanamido]-4-methylpentyl]-1,3-thiazole-4-carboxylic Acid (3Ib)

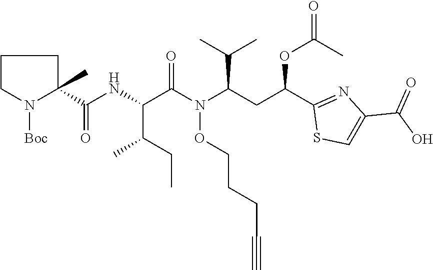 [SMILES: C#CCCCON(C(=O)[C@@H](NC(=O)[C@@]1(C)CCCN1C(=O)OC(C)(C)C)[C@@H](C)CC)[C@H](C[C@@H](OC(C)=O)c1nc(C(=O)O)cs1)C(C)C]

Following General Procedure V from compound 3Hb (65 mg, 0.10 mmol), compound 3Ib (55 mg, 72% yield from 2Hb) was obtained as a white solid after purification by reversed phase flash chromatography (0-100% acetonitrile in aq. TFA (0.01%)). ESI m/z: 693 (M+H)⁺, 593 (M−Boc+H)⁺.

### 2-[(1R,3R)-1-(Acetyloxy)-3-[(2S,3S)-2-{[(2R)-1-[(tert-butoxy)carbonyl]piperidin-2-yl]formamido}-3-methyl-N-(pentyloxy)pentanamido]-4-methylpentyl]-1,3-thiazole-4-carboxylic Acid (3Ic)

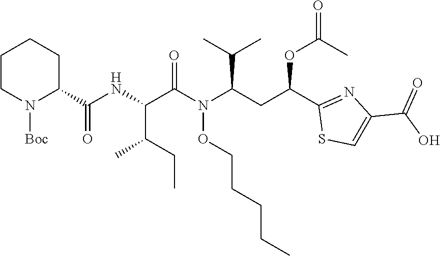 [SMILES: CCCCCON(C(=O)[C@@H](NC(=O)[C@H]1CCCCN1C(=O)OC(C)(C)C)[C@@H](C)CC)[C@H](C[C@@H](OC(C)=O)c1nc(C(=O)O)cs1)C(C)C]

Following General Procedure V from crude compound 3Hc (69 mg), compound 3Ic (63 mg, 65% yield in 2 steps from 2Hc) was obtained as a white solid after purification by reversed phase flash chromatography (0-20% acetonitrile in aq. ammonium bicarbonate (10 mM)). ESI m/z: 719 (M+Na)⁺.

Synthesis of Tubulysin Payloads in Table 4

### P33: (4S)-5-(4-aminophenyl)-4-({2-[(1R,3R)-1-hydroxy-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-2,2-dimethylpentanoic Acid (P33)

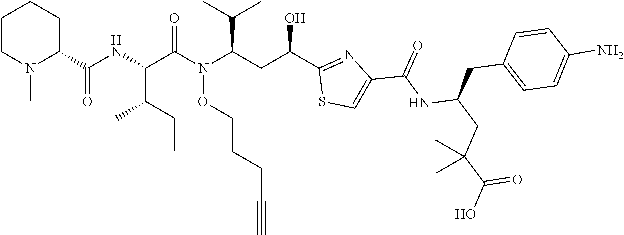 [SMILES: C#CCCCON(C(=O)[C@@H](NC(=O)[C@H]1CCCCN1C)[C@@H](C)CC)[C@H](C[C@@H](O)c1nc(C(=O)N[C@@H](Cc2ccc(N)cc2)CC(C)(C)C(=O)O)cs1)C(C)C]

To a solution of P34 (9.4 mg, 11 μmol, see below) in aq. THF (80 vol %, 2.0 mL) was added lithium hydroxide (5.5 mg, 0.23 mmol). The mixture was stirred at room temperature overnight and monitored by LCMS. The reaction mixture was then acidified by aq. HCl (1 M) to pH 3, and extracted with ethyl acetate. The combined organic solution was dried over sodium sulfate and concentrated in vacuo. The residue was purified by prep-HPLC (0-100% acetonitrile in aq. ammonium bicarbonate (10 mM)) to give payload P33 (8.0 mg, 90% yield) as a white solid. ESI m/z: 783.4 (M+H)⁺. ¹H NMR (400 MHz, DMSO_{d6}) δ 8.08 (s, 1H), 7.64 (d, J=9.6 Hz, 1H), 6.82 (d, J=8.4 Hz, 2H), 6.45 (d, J=8.0 Hz, 2H), 6.33-6.32 (br s, 1H), 4.85-4.83 (br s, 1H), 4.77-4.75 (m, 1H), 4.73-4.66 (m, 1H), 4.31-4.29 (m, 1H), 4.14-4.07 (m, 3H), 2.84-2.81 (m, 2H), 2.68-2.64 (m, 1H), 2.45-2.31 (m, 4H), 2.07 (s, 3H), 2.01-1.95 (m, 3H), 1.91-1.81 (m, 5H), 1.61-1.58 (m, 3H), 1.54-1.35 (m, 5H), 1.23 (s, 1H), 1.19-1.07 (m, 2H), 1.02-0.99 (m, 6H), 0.96-0.90 (m, 9H), 0.88-0.80 (m, 3H) ppm.

### P34: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-(4-aminophenyl)-2,2-dimethylpentanoic Acid (P34)

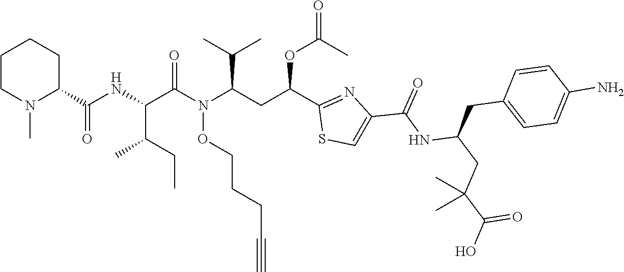 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C]

Following General Procedure VI from compound 3Ia with compound TUPb, payload P34 (5.3 mg, 27% yield) was obtained as a white solid. ESI m/z: 413.3 (M/2+H)⁺, 825.3 (M+H)⁺(30%). ¹H NMR (500 MHz, DMSO_{d6}) δ 8.16 (s, 1H), 7.78 (d, J=8.5 Hz, 1H), 7.56 (d, J=10.0 Hz, 1H), 6.81 (d, J=8.5 Hz, 2H), 6.44 (d, J=8.0 Hz, 2H), 5.81 (d, J=11.0 Hz, 1H), 4.90-4.74 (m, 3H), 4.26-4.23 (m, 1H), 4.15-4.05 (m, 3H), 2.85-2.82 (m, 2H), 2.77 (br s, 1H), 2.68-2.64 (m, 1H), 2.36-2.31 (m, 3H), 2.13 (s, 3H), 2.09 (s, 3H), 2.03-1.96 (m, 2H), 1.89-1.78 (m, 4H), 1.62-1.35 (m, 9H), 1.19-1.05 (m, 2H), 1.04 (s, 3H), 1.00 (s, 3H), 0.96 (d, J=5.5 Hz, 3H), 0.89-0.82 (m, 9H) ppm.

### P35: (4S)-4-({2-[(1R,3R)-1-(acetyloxy)-4-methyl-3-[(2S,3S)-3-methyl-2-{[(2R)-2-methylpyrrolidin-2-yl]formamido}-N-(pent-4-yn-1-yloxy)pentanamido]pentyl]-1,3-thiazol-4-yl}formamido)-5-(4-amino-3-fluorophenyl)-2,2-dimethylpentanoic Acid (P35)

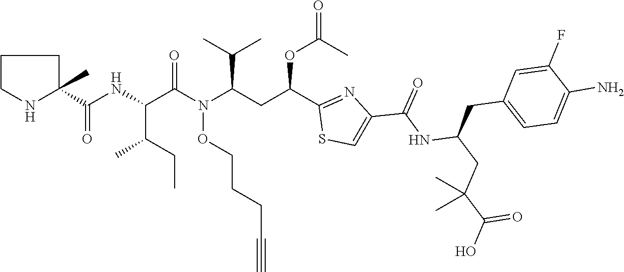 [SMILES: C#CCCCON(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C]

Following General Procedure VI from compound 3Ib (30 mg, 43 μmol) with TUPa, Boc-P35 (30 mg) was obtained as a white solid. Boc-P35 was dissolved in DCM (2 mL). To the solution was added TFA (0.5 mL) and the mixture was stirred at room temperature for an hour until Boc was totally removed according to LCMS. The resulting mixture was concentrated in vacuo and the residue was purified by prep-HPLC (0-100% acetonitrile in aq. ammonium bicarbonate (10 mM)) to give P35 (15 mg, 42% yield from 3Ib) as a white solid. ESI m/z: 415 (M/2+H)⁺, 829.5 (M+H). ¹H NMR (400 MHz, DMSO_{d6}) δ 8.23 (d, J=10.1 Hz, 1H), 8.15 (s, 1H), 7.65 (d, J=9.0 Hz, 1H), 6.80-6.75 (m, 1H), 6.68-6.59 (m, 2H), 5.84 (d, J=8.7 Hz, 1H), 4.88 (s, 2H), 4.73-4.67 (m, 1H), 4.21-4.06 (m, 4H), 3.00-2.89 (m, 1H), 2.83 (t, J=2.5 Hz, 1H), 2.71-2.54 (m, 3H), 2.46 (s, 1H), 2.42-2.23 (m, 4H), 2.13 (s, 3H), 2.02-1.98 (m, 2H), 1.94-1.53 (m, 7H), 1.51-1.38 (m, 3H), 1.27 (s, 3H), 1.08 (s, 3H), 1.05 (s, 3H), 0.95 (d, J=6.6 Hz, 3H), 0.88-0.82 (m, 9H) ppm.

### P36: (4S)-4-({2-[(1R,3R)-1-(acetyloxy)-4-methyl-3-[(2S,3S)-3-methyl-N-(pentyloxy)-2-[(2R)-piperidin-2-ylformamido]pentanamido]pentyl]-1,3-thiazol-4-yl}formamido)-5-(4-aminophenyl)-2,2-dimethylpentanoic Acid (P36)

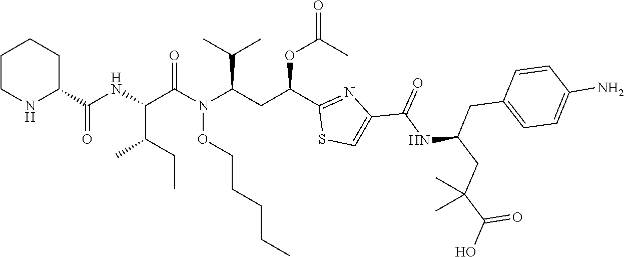 [SMILES: CCCCCON(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C]

Following General Procedure VI from compound 3Ic (30 mg, 43 μmol), compound Boc-P36 (19 mg, ESI m/z: 915.5 (M+H)⁺) was obtained after purification by reversed phase flash chromatography (0-100% acetonitrile in aq. TFA (0.01%)). To a solution of Boc-P36 (19 mg) in DCM (0.6 mL) was added TFA (0.2 mL), and the mixture was stirred at room temperature for 3 hours until Boc was totally removed, according to LCMS. The resulting mixture was concentrated in vacuo and the residue was purified by prep-HPLC (0-30% acetonitrile in aq. ammonium bicarbonate (10 mM)) to give P36 (4.4 mg, 13% yield from 3Ic) as a white solid. ESI m/z: 815.5 (M+H)⁺, 408 (M/2+H)⁺. ¹H NMR (400 MHz, DMSO_{d6}) δ 8.17 (s, 1H), 7.83-7.68 (m, 2H), 7.32-7.25 (m, 2H), 6.81 (d, J=8.3 Hz, 2H), 6.44 (d, J=8.3 Hz, 2H), 5.81 (dd, J=10.4 and 1.9 Hz, 1H), 4.90-4.76 (m, 3H), 4.20-4.07 (m, 3H), 4.05-3.89 (m, 3H), 2.90-2.85 (m, 2H), 2.70-2.61 (m, 2H), 2.39-2.28 (m, 3H), 2.13 (s, 3H), 2.07-1.95 (m, 3H), 1.93-1.82 (m, 2H), 1.81-1.71 (m, 2H), 1.70-1.55 (m, 6H), 1.51-1.40 (m, 3H), 1.03 (s, 3H), 1.01 (s, 3H), 0.97 (d, J=6.6 Hz, 3H), 0.90-0.79 (m, 12H) ppm.

### P51: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-(4-hydroxyphenyl)-2,2-dimethylpentanoic Acid (P51)

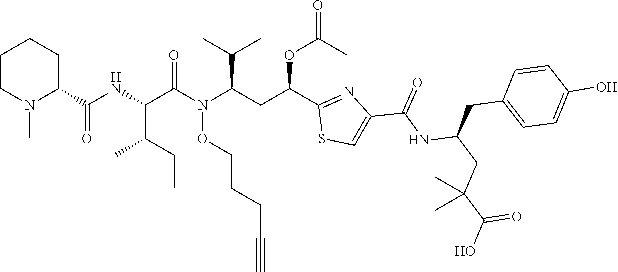 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C]

Following General Procedure VI from compound 3Ia with TUPd, payload P51 (15 mg, 12% yield from 3Ia) was obtained as a white solid. ESI m/z 826.5 (M+H)⁺. ¹H NMR (400 MHz, DMSO_{d6}) δ 9.16 (s, 1H), 8.16 (s, 1H), 7.65 (d, J=8.4 Hz, 1H), 7.59 (d, J=9.6 Hz, 1H), 6.95 (d, J=8.4 Hz, 2H), 6.62 (d, J=8.4 Hz, 2H), 5.82 (d, J=10.0 Hz, 1H), 4.76 (t, J=8.4 Hz, 1H), 4.23-4.20 (m, 2H), 4.09-4.01 (m, 2H), 2.90-2.80 (m, 2H), 2.73-2.68 (m, 1H), 2.63-2.58 (m, 1H), 2.39-2.31 (m, 3H), 2.14-2.06 (m, 6H), 2.01-1.87 (m, 3H), 1.84-1.80 (m, 3H), 1.66-1.61 (m, 3H), 1.56-1.53 (m, 1H), 1.46-1.36 (m, 3H), 1.22-1.11 (m, 2H), 1.05-1.03 (m, 7H), 0.96 (d, J=6.4 Hz, 3H), 0.88-0.81 (m, 10H) ppm.

TABLE 5-1
Compound List of Aminoacid-P34

| | | | | | | HPLC | HPLC |
|---|---|---|---|---|---|---|---|
| | | | | | Mass | purity | RT |
| # | Structures | cLogP | MF | MW | m/z | (%) | (min) |
| P37 | 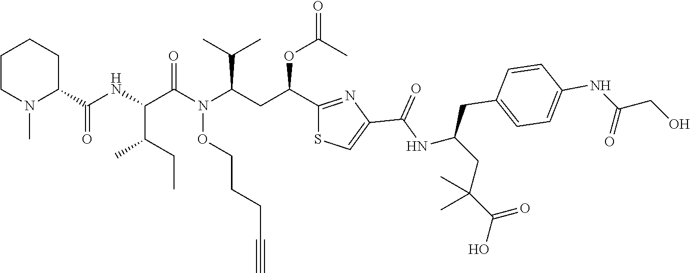 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CO)cc2)CC(C)(C)C(=O)O)cs1)C(C)C] | 2.47 | C₄₅H₆₆N₆O₁₀S | 883.1 | 442 (M/2 + H) | 99 | 7.03 (B) |
| P39 | 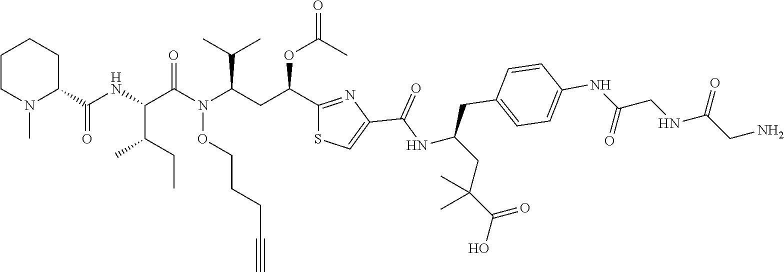 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)CN)cc2)CC(C)(C)C(=O)O)cs1)C(C)C] | 1.27 | C₄₇H₇₀N₈O₁₀S | 939.2 | 470 (M/2 + H) | 99 | 6.54 (B) |
| P41 | 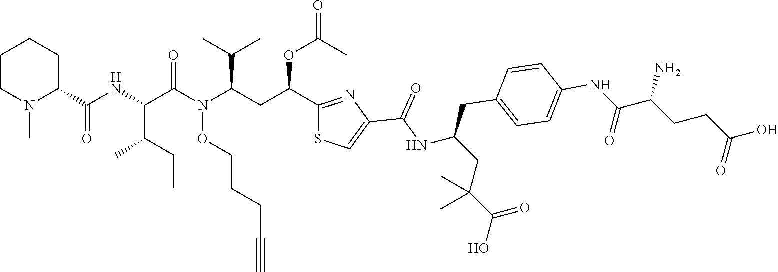 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](N)CCC(=O)O)cc2)CC(C)(C)C(=O)O)cs1)C(C)C] | 0.39 | C₄₈H₇₁N₇O₁₁S | 954.2 | 478 (M/2 + H) | 96 | 5.95 (A) |

TABLE 5-2
Modification on Aminoacid-P34

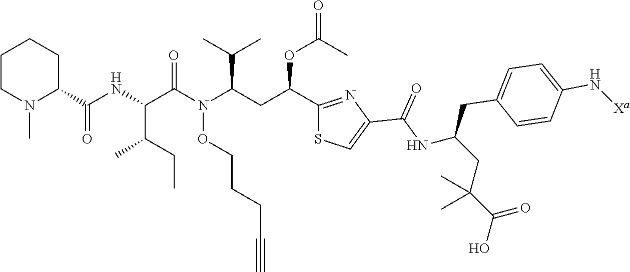

| | | | HCT- | HCT-15 with |
|---|---|---|---|---|
| | Structures | | 15 IC₅₀ | verapamil IC₅₀ |
| # | X^{a} | cLogP | (nM) | (nM) |
| P37 | COCH₂OH | 2.47 | 102 | |
| P39 | GlyGly | 1.27 | 0.62 | |
| P41 | (D)-Glu | 0.39 | 82.7 | |

Synthesis of Tubulysin Payloads P37-P41 in Table 5

### P37: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-[4-(2-hydroxyacetamido)phenyl]-2,2-dimethylpentanoic Acid (P37)

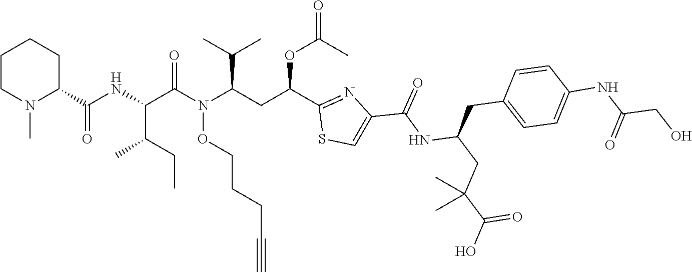 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CO)cc2)CC(C)(C)C(=O)O)cs1)C(C)C]

Following General Procedure VI from compound 3Ia (30 mg, 50 μmol) with intermediate TUPk (15 mg, 51 μmol), payload P37 (21 mg, 48% yield) was obtained as a white solid. ESI m/z: 883 (M+H)⁺. ¹H NMR (400 MHz, DMSO_{d6}) δ 9.54 (s, 1H), 8.16 (s, 1H), 7.77 (d, J=9.0 Hz, 1H), 7.60-7.53 (m, 3H), 7.10 (d, J=8.5 Hz, 2H), 5.82 (dd, J=10.8 and 1.7 Hz, 1H), 5.63 (s, 1H), 4.80-4.70 (m, 1H), 4.29-4.20 (m, 2H), 4.11-4.01 (m, 2H), 3.95 (s, 2H), 2.88-2.65 (m, 5H), 2.41-2.26 (m, 4H), 2.13 (s, 3H), 2.10 (s, 3H), 2.05-1.89 (m, 4H), 1.86-1.78 (m, 3H), 1.70-1.59 (m, 3H), 1.56-1.51 (m, 1H), 1.48-1.34 (m, 3H), 1.23 (s, 1H), 1.19-1.13 (m, 1H), 1.07 (s, 3H), 1.04 (s, 3H), 0.96 (d, J=6.6 Hz, 3H), 0.90-0.80 (m, 9H) ppm.

### P38: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-[4-(2-aminoacetamido)phenyl]-2,2-dimethylpentanoic Acid (P38)

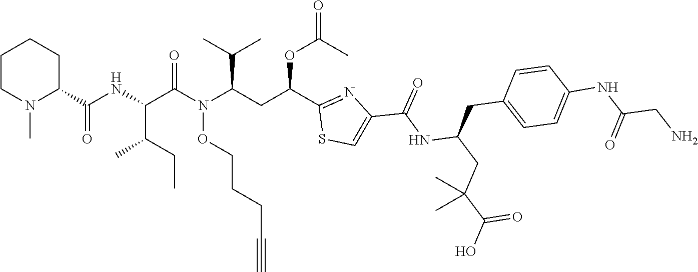 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C]

Following General Procedure VI from 3Ia (15 mg, 25 μmol) with intermediate TUPg, Fmoc-P38 (6.1 mg, ESI m/z: 553 (M/2+H)⁺) was obtained as a white solid. Fmoc-P38 was dissolved in DMF (2 mL). To the solution was added piperidine (20 μL) and the reaction mixture was stirred at room temperature for 2 hours until Fmoc was totally removed according to LCMS. The resulting mixture was directly purified by prep-HPLC (0-100% acetonitrile in aq. TFA (0.01%)) to give P38 (2.8 mg, 11% yield in 3 steps from 3Ia, TFA salt) as a white solid. ESI m/z: 442 (M/2+H)⁺. ¹H NMR (400 MHz, DMSO_{d6}) δ 10.33 (s, 1H), 8.18 (s, 1H), 8.14-8.04 (m, 3H), 7.83 (d, J=9.5 Hz, 1H), 7.45 (d, J=8.5 Hz, 2H), 7.17 (d, J=8.5 Hz, 2H), 5.85-5.79 (m, 1H), 4.78-4.72 (m, 1H), 4.32-4.18 (m, 3H), 4.12-4.01 (m, 2H), 3.74 (s, 2H), 2.85 (t, J=2.5 Hz, 1H), 2.83-2.75 (m, 2H), 2.72-2.65 (m, 2H), 2.64-2.57 (m, 2H), 2.41-2.30 (m, 5H), 2.13 (s, 3H), 2.09-2.00 (m, 3H), 2.01-1.92 (m, 2H), 1.89-1.82 (m, 3H), 1.78-1.72 (m, 2H), 1.71-1.64 (m, 2H), 1.58-1.51 (m, 1H), 1.49-1.35 (m, 3H), 1.17-1.12 (m, 1H), 1.06 (s, 3H), 1.04 (s, 3H), 0.97 (d, J=6.5 Hz, 3H), 0.91-0.87 (m, 4H), 0.84 (t, J=7.4 Hz, 3H) ppm.

### P39: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-{4-[2-(2-aminoacetamido)acetamido]phenyl}-2,2-dimethylpentanoic Acid (P39)

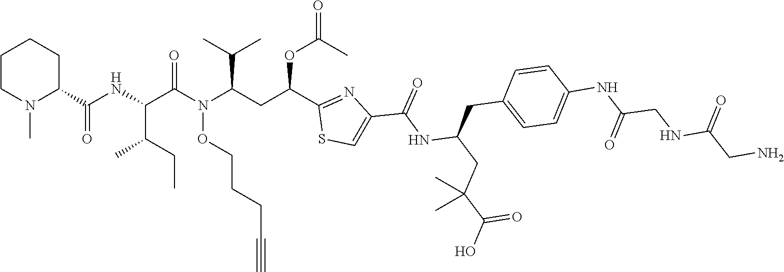 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)CN)cc2)CC(C)(C)C(=O)O)cs1)C(C)C]

Following General Procedure VI from 3Ia (60 mg, 99 μmol) with intermediate TUPh, Fmoc-P39 (70 mg, ESI m/z: 1162 (M+H)⁺) was obtained as a white solid. Fmoc-P39 was dissolved in DMF (2 mL). To the solution was added piperidine (18 mg, 0.21 mmol) and the reaction mixture was stirred at room temperature for 2 hours until Fmoc was totally removed according to LCMS. The resulting mixture was purified directly by prep-HPLC (10-95% acetonitrile in aq. ammonium bicarbonate (10 mM)) to give P39 (24 mg, 26% yield in 3 steps from 3Ia) as a white solid. ESI m/z: 470 (M/2+H)⁺, 939 (M+H)⁺. ¹H NMR (400 MHz, DMSO_{d6}) δ 9.89 (s, 1H), 8.21 (s, 1H), 8.16 (s, 1H), 7.84 (d, J=8.0 Hz, 1H), 7.58 (d, J=9.2 Hz, 1H), 7.45 (d, J=8.4 Hz, 2H), 7.10 (d, J=8.4 Hz, 2H), 5.81 (d, J=10.0 Hz, 1H), 4.76 (t, J=8.4 Hz, 1H), 4.29-4.20 (m, 2H), 4.10-4.01 (m, 2H), 3.90 (s, 2H), 3.16 (s, 2H), 2.88-2.76 (m, 3H), 2.71-2.64 (m, 1H), 2.40-2.30 (m, 3H), 2.13 (s, 3H), 2.10 (m, 3H), 2.00-1.90 (m, 3H), 1.86-1.78 (m, 3H), 1.68-1.58 (m, 3H), 1.54-1.49 (m, 1H), 1.47-1.32 (m, 3H), 1.19-1.10 (m, 1H), 1.06-1.02 (m, 7H), 0.96 (d, J=6.4 Hz, 3H), 0.90-0.80 (m, 11H) ppm.

### P40: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-{4-[(2S)-2-amino-4-carboxybutanamido]phenyl}-2,2-dimethylpentanoic Acid (P40)

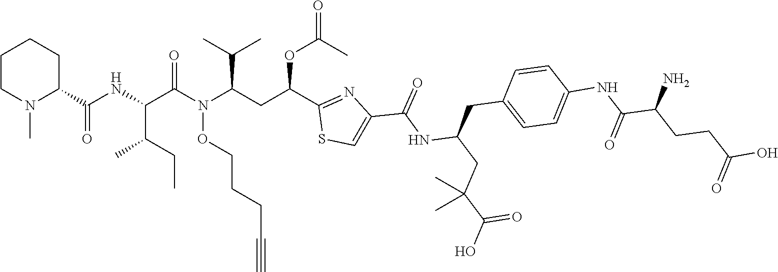 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@@H](N)CCC(=O)O)cc2)CC(C)(C)C(=O)O)cs1)C(C)C]

Following General Procedure VI from 3Ia (20 mg, 33 μmol) with intermediate TUPi, Fmoc-P40 (30 mg, ESI m/z: 589 (M/2+H)⁺) was obtained as a white solid. Fmoc-P40 was dissolved in DMF (2 mL). To the solution was added piperidine (5.0 mg, 59 μmol) and the reaction mixture was stirred at room temperature for an hour until Fmoc was totally removed according to LCMS. The resulting mixture was purified directly by prep-HPLC (0-100% acetonitrile in aq. ammonium bicarbonate (10 mM)) to give P40 (15 mg, 48% yield in 3 steps from 3Ia) as a white solid. ESI m/z: 478 (M/2+H)⁺. ¹H NMR (400 MHz, DMSO_{d6}) δ 8.16 (s, 1H), 7.87 (d, J=8.6 Hz, 1H), 7.56 (d, J=9.4 Hz, 1H), 7.50 (d, J=8.4 Hz, 2H), 7.11 (d, J=8.5 Hz, 2H), 5.82 (d, J=9.3 Hz, 1H), 4.75 (t, J=8.4 Hz, 1H), 4.27-4.19 (m, 3H), 4.09-4.02 (m, 3H), 2.91-2.76 (m, 4H), 2.72-2.64 (m, 1H), 2.44-2.22 (m, 6H), 2.13 (s, 3H), 2.10 (s, 3H), 2.04-1.74 (m, 7H), 1.69-1.60 (m, 4H), 1.51-1.35 (m, 5H), 1.05 (s, 3H), 1.03 (s, 3H), 0.96 (d, J=6.6 Hz, 3H), 0.88-0.81 (m, 9H) ppm.

### P41: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-{4-[(2R)-2-amino-4-carboxybutanamido]phenyl}-2,2-dimethylpentanoic Acid (P41)

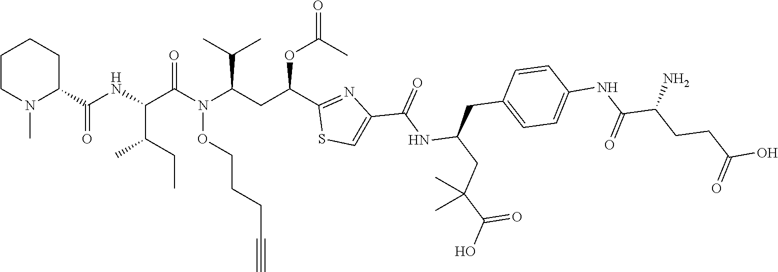 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](N)CCC(=O)O)cc2)CC(C)(C)C(=O)O)cs1)C(C)C]

Following General Procedure VI from 3Ia (80 mg, 0.13 mmol) with intermediate TUPj, Fmoc-P40 (65 mg, ESI m/z: 589 (M/2+H)⁺) was obtained as a white solid. Fmoc-P40 was dissolved in DMF (2 mL). To the solution was added piperidine (5.0 mg, 59 μmol) and the reaction mixture was stirred at room temperature for an hour until Fmoc was totally removed according to LCMS. The resulting mixture was purified directly by prep-HPLC (0-100% acetonitrile in aq. ammonium bicarbonate (10 mM)) to give P40 (30 mg, 24% yield in 3 steps from 3Ia) as a white solid. ESI m/z: 478 (M/2+H)⁺. ¹H NMR (400 MHz, DMSO_{d6}) δ 8.16 (s, 1H), 7.78 (d, J=8.6 Hz, 1H), 7.62 (d, J=9.4 Hz, 1H), 7.48 (d, J=8.4 Hz, 2H), 7.11 (d, J=8.5 Hz, 2H), 5.82 (d, J=9.3 Hz, 1H), 4.75 (t, J=8.4 Hz, 1H), 4.26-4.05 (m, 6H), 2.96-2.50 (m, 5H), 2.44-2.22 (m, 6H), 2.13 (s, 3H), 2.10 (s, 3H), 2.04-1.74 (m, 7H), 1.69-1.60 (m, 4H), 1.51-1.35 (m, 5H), 1.05 (s, 3H), 1.03 (s, 3H), 0.96 (d, J=6.6 Hz, 3H), 0.88-0.81 (m, 9H) ppm.

TABLE 6-1
Compound List of N-acylsulfonamides

| | | | | | | HPLC | HPLC |
|---|---|---|---|---|---|---|---|
| | | | | | Mass | purity | RT |
| # | Structures | cLogP | MF | MW | m/z | (%) | (min) |
| P42 | 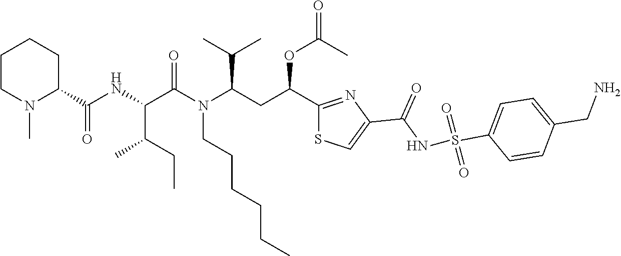 [SMILES: CCCCCCN(C(=O)[C@@H](NC(=O)[C@H]1CCCCN1C)[C@@H](C)CC)[C@H](C[C@@H](OC(C)=O)c1nc(C(=O)NS(=O)(=O)c2ccc(CN)cc2)cs1)C(C)C] | 4.19 | C₃₈H₆₀N₆O₇S₂ | 777.05 | 777 (M + H) | 96 | 8.24 (B) |
| P43 | 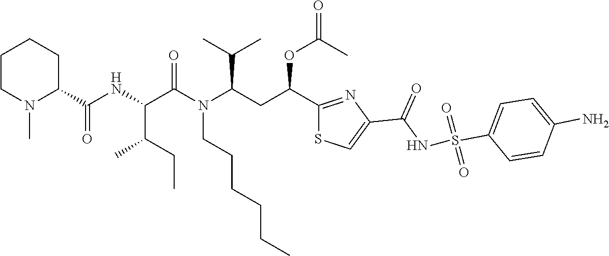 [SMILES: CCCCCCN(C(=O)[C@@H](NC(=O)[C@H]1CCCCN1C)[C@@H](C)CC)[C@H](C[C@@H](OC(C)=O)c1nc(C(=O)NS(=O)(=O)c2ccc(N)cc2)cs1)C(C)C] | 3.80 | C₃₇H₅₈N₆O₇S₂ | 763.03 | 763 (M + H) | 92 | 8.06 (B) |
| P44 | 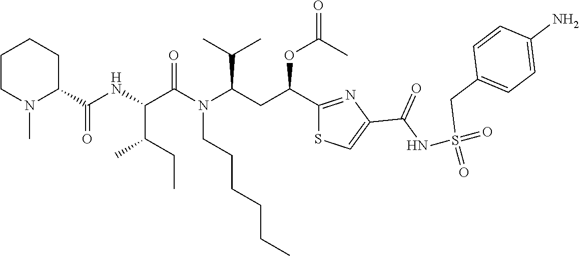 | 3.64 | C₃₈H₆₀N₆O₇S₂ | 777.05 | 777 (M + H) | 95 | 7.46 (B) |
| P45 | 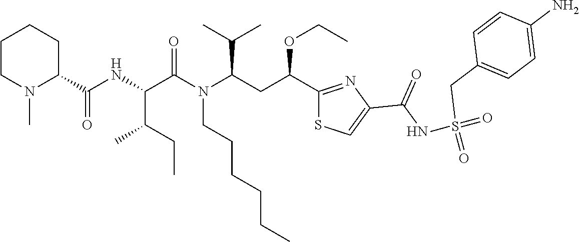 | 4.19 | C₃₈H₆₂N₆O₆S₂ | 763.07 | 763 (M + H) | 96 | 7.78 (B) |
| P46 | 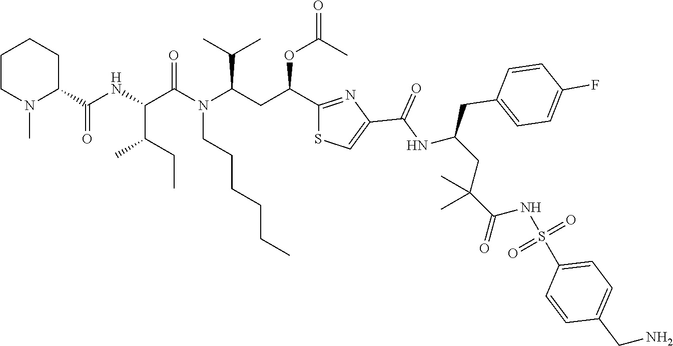 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)NS(=O)(=O)c2ccc(CN)cc2)cs1)C(C)C] | 6.93 | C₅₁H₇₆FN₇O₈S₂ | 998.33 | 500 (M/2 + H) | 98 | 8.56 (B) |
| P47 | 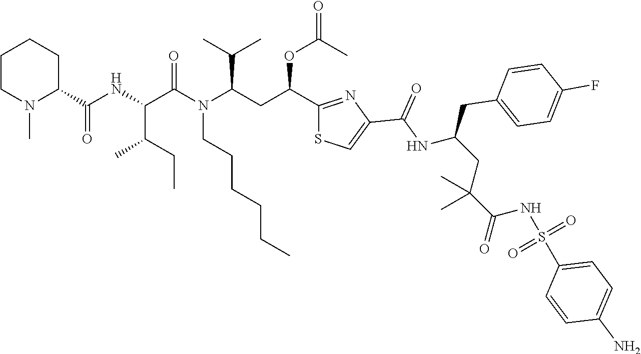 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)NS(=O)(=O)c2ccc(N)cc2)cs1)C(C)C] | 6.66 | C₅₀H₇₄FN₇O₈S₂ | 984.30 | 985 (M + H) | 99 | 8.44 (B) |
| P48 | 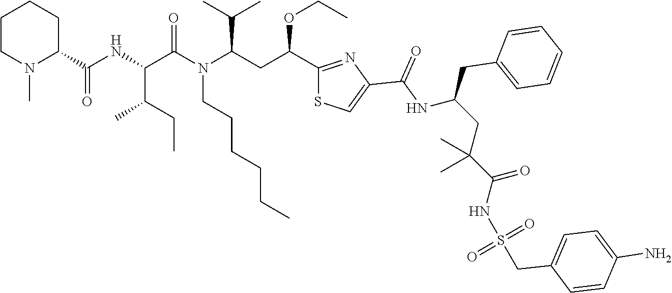 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccccc2)CC(C)(C)C(=O)NS(=O)(=O)Cc2ccc(N)cc2)cs1)C(C)C] | 6.79 | C₅₁H₇₉N₇O₇S₂ | 966.36 | 966 (M + H) | 99 | 7.83 (B) |
| P49 | 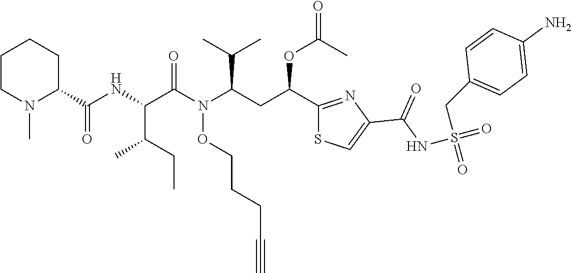 [SMILES: C#CCCCON(C(=O)[C@@H](NC(=O)[C@H]1CCCCN1C)[C@@H](C)CC)[C@H](C[C@@H](OC(C)=O)c1nc(C(=O)NS(=O)(=O)Cc2ccc(N)cc2)cs1)C(C)C] | 2.53 | C₃₇H₅₄N₆O₈S₂ | 774.99 | 775 (M + H) | 99 | 7.54 (B) |

TABLE 6-2
N-acylsulfonamides

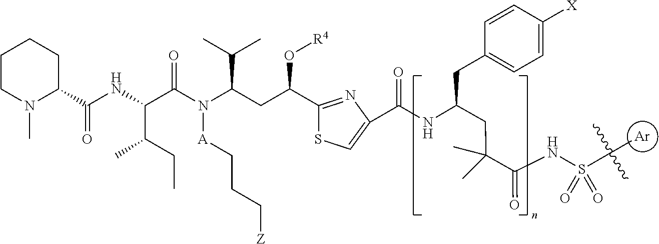

| | | | | | | | | | HCT-15 |
|---|---|---|---|---|---|---|---|---|---|
| | | | | | | | | | with |
| | Structures | | | | | | | HCT-15 | verapamil |
| # | A | Z | R⁴ | n | X | Ar | cLogP | IC₅₀ (nM) | IC₅₀ (nM) |
| P42 | CH₂ | Et | Ac | 0 | / | 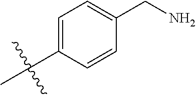 [SMILES: *c1ccc(CN)cc1] | 4.19 | >100 | 48.4 |
| P43 | CH₂ | Et | Ac | 0 | / | 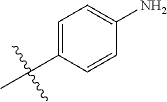 [SMILES: *c1ccc(N)cc1] | 3.80 | 4.99 | 1.42 |
| P44 | CH₂ | Et | Ac | 0 | / | 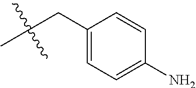 [SMILES: *Cc1ccc(N)cc1] | 3.64 | 25.8 | |
| P45 | CH₂ | Et | Et | 0 | / | 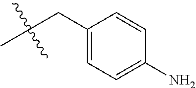 [SMILES: *Cc1ccc(N)cc1] | 4.19 | 75.7 | |
| P46 | CH₂ | Et | Ac | 1 | F | 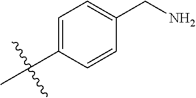 [SMILES: *c1ccc(CN)cc1] | 6.93 | >100 | |
| P47 | CH₂ | Et | Ac | 1 | F | 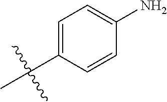 [SMILES: *c1ccc(N)cc1] | 6.66 | 53.5 | |
| P48 | CH₂ | Et | Et | 1 | H | 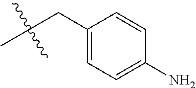 [SMILES: *Cc1ccc(N)cc1] | 6.79 | 65.8 | |
| P49 | O | C≡CH | Ac | 0 | / | 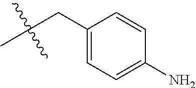 [SMILES: *Cc1ccc(N)cc1] | 2.53 | 163 | |

Synthesis of Tubulysin Payloads P37-P41 in Table 5

### P42: (1R,3R)-1-(4-{[4-(aminomethyl)benzenesulfonyl]carbamoyl}-1,3-thiazol-2-yl)-3-[(2S,3S)—N-hexyl-3-methyl-2-{[(2R)-1-methylpiperidin-2-yl]formamido}pentanamido]-4-methylpentyl Acetate (P42)

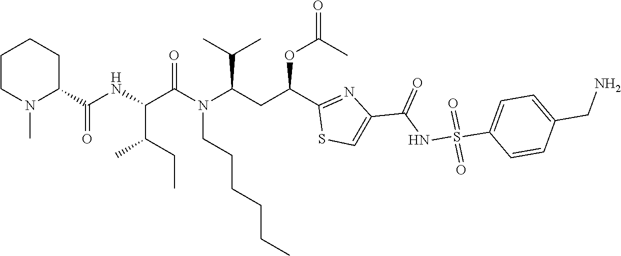 [SMILES: CCCCCCN(C(=O)[C@@H](NC(=O)[C@H]1CCCCN1C)[C@@H](C)CC)[C@H](C[C@@H](OC(C)=O)c1nc(C(=O)NS(=O)(=O)c2ccc(CN)cc2)cs1)C(C)C]

Following General Procedure VII for N-acylsulfonamides from compound 3Fa with sulfonamide SULa, payload P42 (8 mg, 21% yield from 3Fa) was obtained as a white solid. ESI m/z 777 (M+H)⁺. ¹H NMR (400 MHz, DMSO_{d6}) δ 8.23 (s, 1H), 7.92 (s, 1H), 7.82 (d, J=8.4 Hz, 2H), 7.75 (br s, 1H), 7.44 (d, J=8.4 Hz, 2H), 5.54 (d, J=13.2 Hz, 1H), 4.48 (t, J=9.2 Hz, 1H), 4.05 (s, 2H), 3.62-3.57 (m, 1H), 3.02-2.94 (m, 1H), 2.85 (d, J=11.2 Hz, 1H), 2.58 (br s, 1H), 2.21-1.99 (m, 9H), 1.88-1.82 (m, 2H), 1.64-1.63 (m, 3H), 1.53-1.40 (m, 5H), 1.37-1.24 (m, 7H), 1.18-1.05 (m, 2H), 0.92 (d, J=6.4 Hz, 3H), 0.88-0.80 (m, 9H), 0.69 (br s, 3H) ppm.

### P43: (1R,3R)-1-{4-[(4-aminobenzenesulfonyl)carbamoyl]-1,3-thiazol-2-yl}-3-[(2S,3S)—N-hexyl-3-methyl-2-{[(2R)-1-methylpiperidin-2-yl]formamido}pentanamido]-4-methylpentyl Acetate (P43)

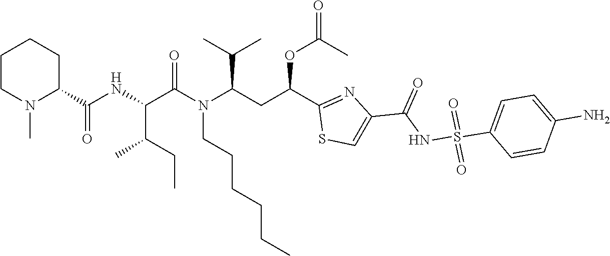 [SMILES: CCCCCCN(C(=O)[C@@H](NC(=O)[C@H]1CCCCN1C)[C@@H](C)CC)[C@H](C[C@@H](OC(C)=O)c1nc(C(=O)NS(=O)(=O)c2ccc(N)cc2)cs1)C(C)C]

Following General Procedure VII from compound 3Fa with sulfonamide SULb, payload P43 (3 mg, 34% yield from 3Fa) was obtained as a white solid. ESI m/z 763 (M+H)⁺. ¹H NMR (400 MHz, DMSO_{d6}) δ 7.99 (s, 1H), 7.51 (d, J=8.4 Hz, 2H), 6.48 (d, J=8.0 Hz, 2H), 5.54 (d, J=11.2 Hz, 2H), 7.51 (t, J=14.4 Hz, 1H), 3.63-3.55 (m, 2H), 3.17-2.99 (m, 7H), 2.14-2.07 (m, 6H), 2.14 (br s, 2H), 1.91-1.39 (m, 9H), 1.35-1.20 (m, 7H), 1.14-1.07 (m, 2H), 0.94-0.79 (m, 15H) ppm.

### P44: (1R,3R)-1-(4-{[(4-aminophenyl)methanesulfonyl]carbamoyl}-1,3-thiazol-2-yl)-3-[(2S,3S)—N-hexyl-3-methyl-2-{[(2R)-1-methylpiperidin-2-yl]formamido}pentanamido]-4-methylpentyl Acetate (P44)

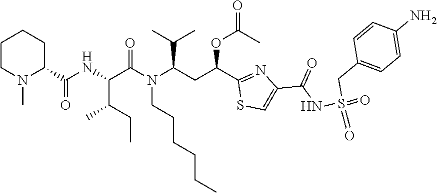 [SMILES: CCCCCCN(C(=O)[C@@H](NC(=O)[C@H]1CCCCN1C)[C@@H](C)CC)[C@H](C[C@@H](OC(C)=O)c1nc(C(=O)NS(=O)(=O)Cc2ccc(N)cc2)cs1)C(C)C]

Following General Procedure VII from compound 3Fa with sulfonamide SULc, payload P44 (6.1 mg, 20% yield from 3Fa) was obtained as a white solid. ESI m/z 777 (M+H)⁺. ¹H NMR (400 MHz, DMSO_{d6}) δ 7.93 (s, 1H), 6.90 (d, J=8.3 Hz, 2H), 6.43 (d, J=8.4 Hz, 2H), 5.56 (d, J=9.8 Hz, 1H), 4.51 (t, J=9.4 Hz, 1H), 4.30-4.16 (m, 2H), 3.68-3.57 (m, 1H), 3.09-2.95 (m, 3H), 2.70-2.65 (m, 1H), 2.37-2.30 (m, 1H), 2.25-2.13 (m, 2H), 2.09 (s, 3H), 2.03-1.87 (m, 3H), 1.84-1.70 (m, 2H), 1.69-1.36 (m, 8H), 1.36-1.17 (m, 9H), 1.16-1.03 (m, 2H), 0.94 (d, J=6.5 Hz, 3H), 0.91-0.60 (m, 13H) ppm.

### P45: N-[(4-aminophenyl)methanesulfonyl]-2-[(1R,3R)-1-ethoxy-3-[(2S,3S)—N-hexyl-3-methyl-2-{[(2R)-1-methylpiperidin-2-yl]formamido}pentanamido]-4-methylpentyl]-1,3-thiazole-4-carboxamide (P45)

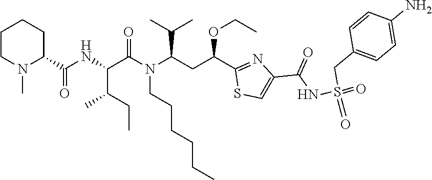 [SMILES: CCCCCCN(C(=O)[C@@H](NC(=O)[C@H]1CCCCN1C)[C@@H](C)CC)[C@H](C[C@@H](OCC)c1nc(C(=O)NS(=O)(=O)Cc2ccc(N)cc2)cs1)C(C)C]

Following General Procedure VII from compound 3Ba (30 mg, 51 μmol) with sulfonamide SULc, payload P45 (5.0 mg, 13% yield from 3Ba) was obtained as a white solid. ESI m/z 763 (M+H)⁺. ¹H NMR (500 MHz, DMSO_{d6}) δ 9.21 (s, 1H), 8.05 (s, 1H), 6.91 (d, J=8.3 Hz, 2H), 6.44 (d, J=8.3 Hz, 2H), 4.52 (t, J=9.6 Hz, 1H), 4.41-4.14 (m, 3H), 3.76-3.67 (m, 1H), 3.34-3.29 (m, 4H), 2.92-2.81 (m, 3H), 2.49-2.37 (m, 3H), 2.07-1.81 (m, 5H), 1.67-1.50 (m, 5H), 1.33-1.23 (m, 9H), 1.11-1.07 (m, 5H), 0.91-0.78 (m, 16H) ppm.

### P46: (1R,3R)-1-(4-{[(2S)-4-{[4-(aminomethyl)benzenesulfonyl]carbamoyl}-1-(4-fluorophenyl)-4,4-dimethylbutan-2-yl]carbamoyl}-1,3-thiazol-2-yl)-3-[(2S,3S)—N-hexyl-3-methyl-2-{[(2R)-1-methylpiperidin-2-yl]formamido}pentanamido]-4-methylpentyl Acetate (P46)

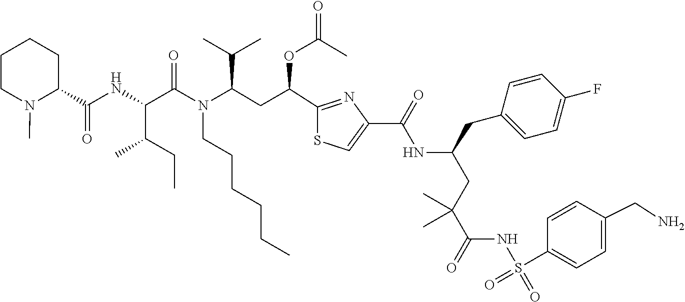 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)NS(=O)(=O)c2ccc(CN)cc2)cs1)C(C)C]

Following General Procedure VII from payload P10 with sulfonamide SULa, payload P46 (6 mg, 67% yield from P10) was obtained as a white solid. ESI m/z 500 (M/2+H)⁺. ¹H NMR (400 MHz, DMSO_{d6}) δ 8.17 (s, 1H), 8.00 (br s, 2H), 7.83 (d, J=8.0 Hz, 1H), 7.72 (d, J=8.0 Hz, 3H), 7.38 (d, J=8.0 Hz, 2H), 7.17-7.13 (m, 2H), 7.04-7.00 (m, 2H), 5.61 (d, J=13.2 Hz, 1H), 4.48 (t, J=8.8 Hz, 1H), 4.14-4.08 (m, 1H), 4.00 (s, 2H), 3.71-3.62 (m, 1H), 3.03-2.67 (m, 5H), 2.34-2.27 (m, 2H), 2.11 (s, 3H), 2.10-1.76 (s, 7H), 1.68-1.52 (m, 10H), 1.50-1.40 (m, 7H), 1.36-1.04 (m, 2H), 0.96 (d, J=6.0 Hz, 3H), 0.92 (d, J=3.6 Hz, 6H), 0.91-0.82 (m, 9H), 0.68 (br s, 3H) ppm. ¹⁹F NMR (376 MHz, DMSO_{d6}) 6-117.5 ppm.

### P47: (1R,3R)-1-(4-{[(2S)-4-[(4-aminobenzenesulfonyl)carbamoyl]-1-(4-fluorophenyl)-4,4-dimethylbutan-2-yl]carbamoyl}-1,3-thiazol-2-yl)-3-[(2S,3S)—N-hexyl-3-methyl-2-{[(2R)-1-methylpiperidin-2-yl]formamido}pentanamido]-4-methylpentyl Acetate (P47)

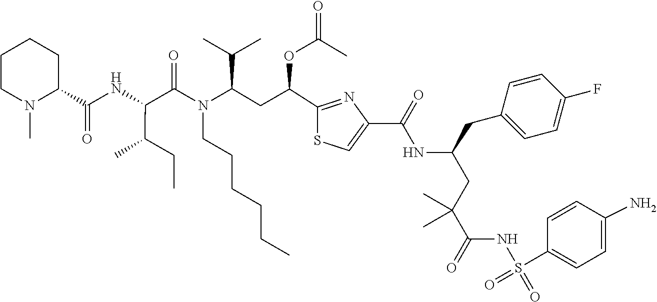 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)NS(=O)(=O)c2ccc(N)cc2)cs1)C(C)C]

Following General Procedure VII from payload P10 with sulfonamide SULb, payload P47 (6.5 mg, 72% yield from P10) was obtained as a white solid. ESI m/z 985 (M+H)⁺. ¹H NMR (400 MHz, DMSO_{d6}) δ 11.11 (s, 1H), 8.14 (s, 1H), 7.79 (s, 2H), 7.48 (d, J=8.4 Hz, 2H), 7.11-7.07 (m, 2H), 7.04-6.99 (m, 2H), 6.53 (d, J=7.6 Hz, 2H), 6.08-6.02 (m, 2H), 5.61 (d, J=12.8 Hz, 1H), 4.48 (t, J=9.2 Hz, 1H), 4.06-4.03 (m, 1H), 3.64 (t, J=8.4 Hz, 1H), 3.01-2.86 (m, 2H), 2.75-2.63 (m, 2H), 2.36-2.30 (m, 1H), 2.20-2.10 (m, 6H), 2.05-1.97 (m, 1H), 1.88-1.84 (m, 2H), 1.78-1.75 (m, 2H), 1.66 (m, 3H), 1.55 (m, 2H), 1.51-1.46 (m, 2H), 1.39-1.36 (m, 1H), 1.25-1.24 (m, 9H), 1.14-1.05 (m, 1H), 1.00-0.95 (m, 9H), 0.84-0.80 (m, 10H), 0.70-0.68 (d, J=6.0 Hz, 3H) ppm. ¹⁹F NMR (400 MHz, DMSO_{d6}) −117.3 ppm.

### P48: (2S,3S)—N-[(1R,3R)-1-(4-{[(2S)-4-{[(4-aminophenyl)methanesulfonyl]carbamoyl}-4,4-dimethyl-1-phenylbutan-2-yl]carbamoyl}-1,3-thiazol-2-yl)-1-ethoxy-4-methylpentan-3-yl]-N-hexyl-3-methyl-2-{[(2R)-1-methylpiperidin-2-yl]formamido}pentanamide (P48)

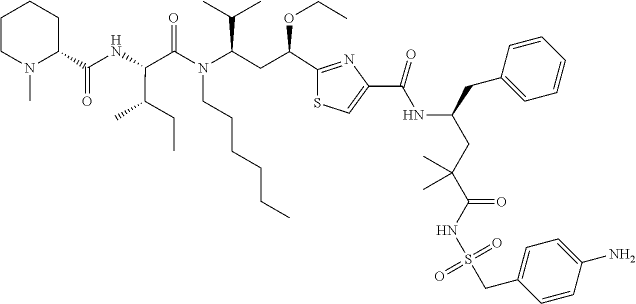 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccccc2)CC(C)(C)C(=O)NS(=O)(=O)Cc2ccc(N)cc2)cs1)C(C)C]

Following General Procedure VII from payload P50 with sulfonamide SULc, payload P48 (5.0 mg, 6.5% yield from P50) was obtained as a white solid. ESI m/z 966 (M+H)⁺. ¹H NMR (500 MHz, DMSO_{d6}) δ 8.16 (s, 1H), 7.80 (br s, 1H), 7.44-7.07 (m, 7H), 6.71 (d, J=7.8 Hz, 2H), 6.38 (d, J=8.1 Hz, 2H), 5.01 (br s, 1H), 4.52 (t, J=9.5 Hz, 1H), 4.24-4.18 (m, 2H), 4.10-4.00 (m, 1H), 3.76-3.65 (m, 1H), 3.01-2.67 (m, 6H), 2.27-2.21 (m, 3H), 1.94-1.81 (m, 6H), 1.70-1.44 (m, 6H), 1.34-1.22 (m, 9H), 1.05-0.98 (m, 10H), 0.91-0.81 (m, 15H), 0.72-0.63 (m, 3H) ppm.

### P49: (1R,3R)-1-(4-{[(4-aminophenyl)methanesulfonyl]carbamoyl}-1,3-thiazol-2-yl)-4-methyl-3-[(2S,3S)-3-methyl-2-{[(2R)-1-methylpiperidin-2-yl]formamido}-N-(pent-4-yn-1-yloxy)pentanamido]pentyl Acetate (P49)

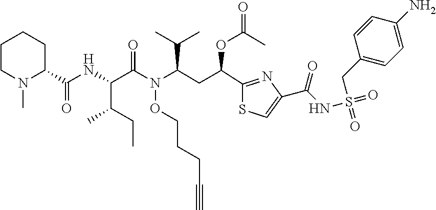 [SMILES: C#CCCCON(C(=O)[C@@H](NC(=O)[C@H]1CCCCN1C)[C@@H](C)CC)[C@H](C[C@@H](OC(C)=O)c1nc(C(=O)NS(=O)(=O)Cc2ccc(N)cc2)cs1)C(C)C]

Following General Procedure VII from intermediate 3Ia (30 mg, 49 μmol) with sulfonamide SULc, payload P49 (1.2 mg, 3.1% yield from 3Ia) was obtained as a white solid after purification by prep-HPLC (0-100% acetonitrile in aq. ammonium bicarbonate (10 mM)) twice. ESI m/z 775 (M+H)⁺. ¹H NMR (400 MHz, methanol_{d4}) δ 8.09 (s, 1H), 7.13 (d, J=8.4 Hz, 2H), 6.64 (d, J=8.4 Hz, 2H), 5.92 (d, J=10.8 Hz, 1H), 5.36 (t, J=4.4 Hz, 1H), 4.82 (d, J=11.2 Hz, 1H), 4.59-4.46 (m, 2H), 4.30-4.24 (m, 1H), 4.02-3.95 (m, 1H), 2.64-2.58 (m, 3H), 2.48-2.39 (m, 1H), 2.38 (t, J=2.8 Hz, 1H), 2.33-2.29 (m, 3H), 2.23-2.17 (m, 1H), 2.15 (s, 3H), 2.10-2.04 (m, 2H), 1.98-1.87 (m, 2H), 1.83-1.52 (m, 7H), 1.22-1.13 (m, 1H), 1.04 (s, 3H), 1.03 (s, 3H), 0.99-0.90 (m, 8H) ppm.

TABLE 7
Structures of Linker-Tubulysins.

| # | Structures | Linker name | Payload |
|---|---|---|---|
| LP2 | 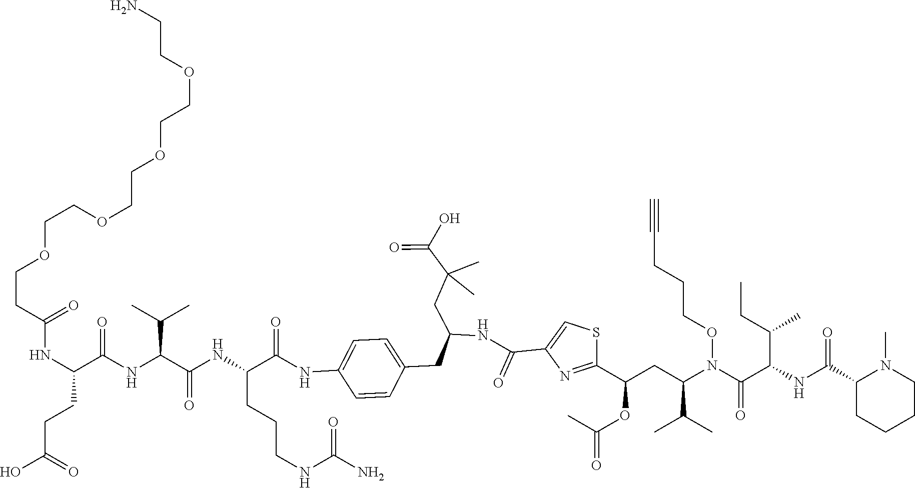 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](CCCNC(N)=O)NC(=O)[C@@H](NC(=O)[C@H](CCC(=O)O)NC(=O)CCOCCOCCOCCOCCN)C(C)C)cc2)CC(C)(C)C(=O)O)cs1)C(C)C] | NH₂-PEG₄- Evc | P34 |
| LP3 | 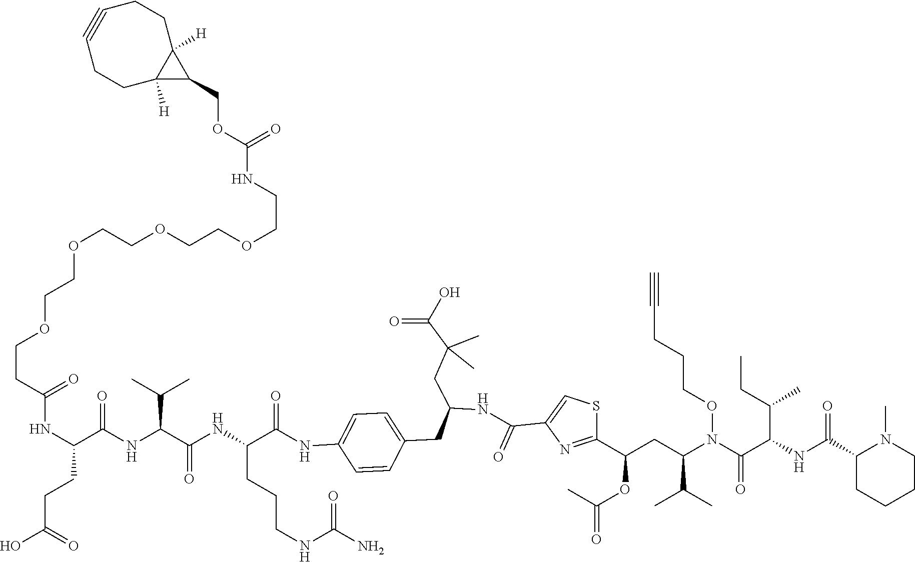 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](CCCNC(N)=O)NC(=O)[C@@H](NC(=O)[C@H](CCC(=O)O)NC(=O)CCOCCOCCOCCOCCNC(=O)OC[C@@H]3[C@@H]4CCC#CCC[C@@H]43)C(C)C)cc2)CC(C)(C)C(=O)O)cs1)C(C)C] | BCN-PEG₄- Evc | P34 |
| LP4 | 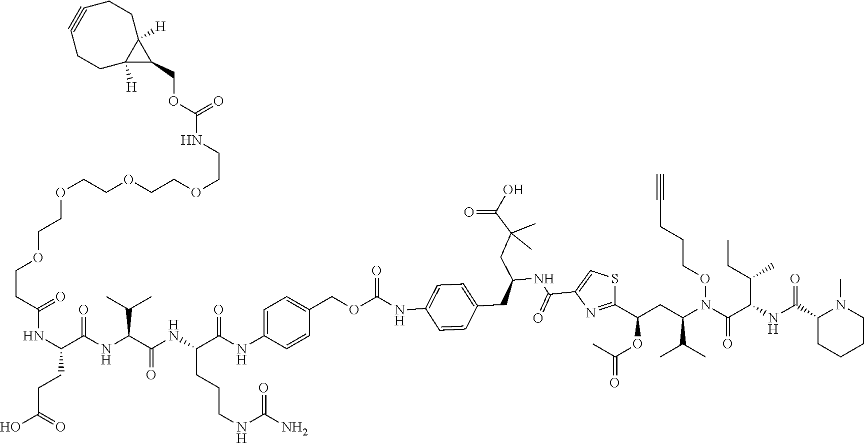 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)OCc3ccc(NC(=O)[C@H](CCCNC(N)=O)NC(=O)[C@@H](NC(=O)[C@H](CCC(=O)O)NC(=O)CCOCCOCCOCCOCCNC(=O)OC[C@@H]4[C@@H]5CCC#CCC[C@@H]54)C(C)C)cc3)cc2)CC(C)(C)C(=O)O)cs1)C(C)C] | BCN-PEG₄- EvcPAB | P34 |
| LP5 | 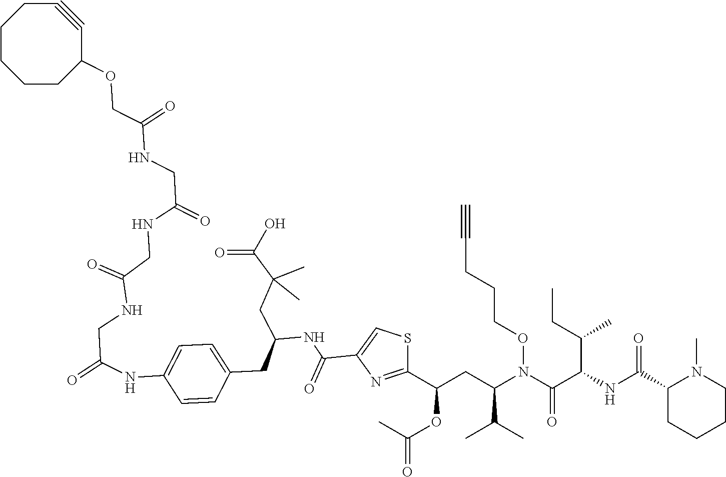 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)CNC(=O)CNC(=O)COC3C#CCCCCC3)cc2)CC(C)(C)C(=O)O)cs1)C(C)C] | COT-GGG | P34 |
| LP6 | 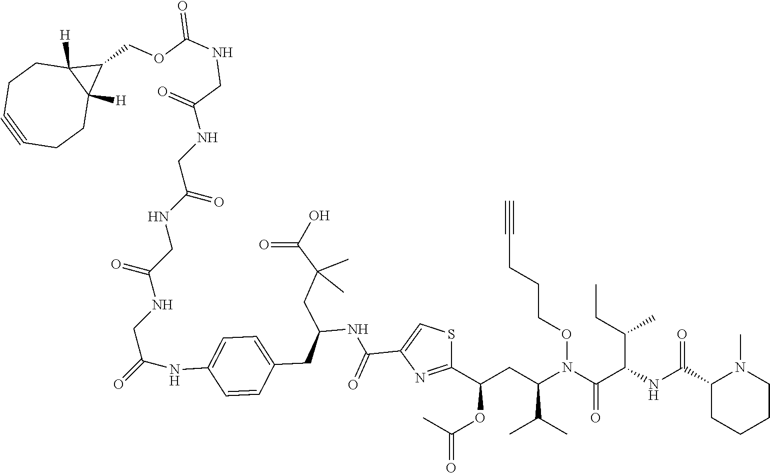 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)CNC(=O)CNC(=O)CNC(=O)OC[C@@H]3[C@@H]4CCC#CCC[C@@H]43)cc2)CC(C)(C)C(=O)O)cs1)C(C)C] | BCN-GGGG (SEQ ID NO: 22) | P34 |
| LP7 | 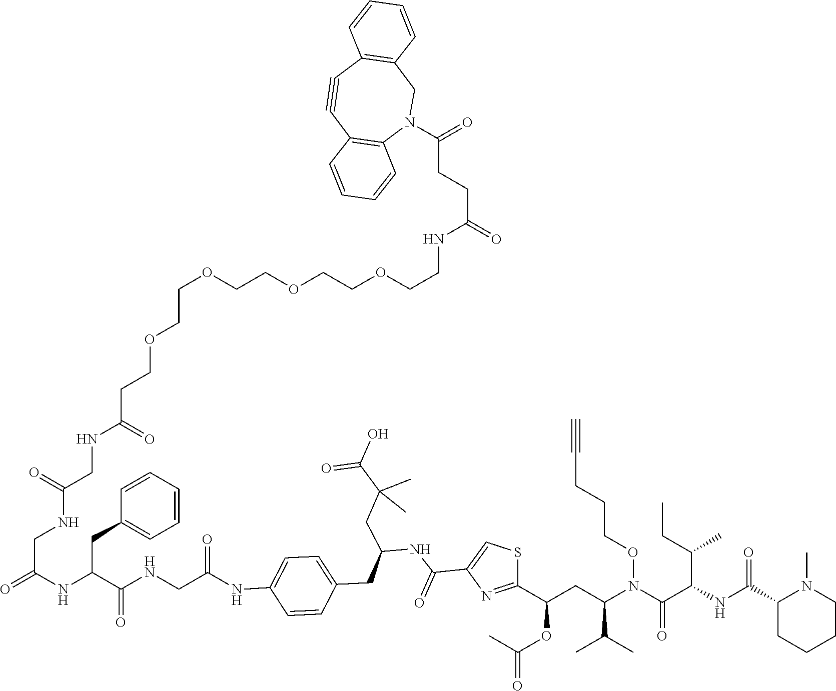 | DIBAC- PEG₄-GGFG (SEQ ID NO: 23) | P34 |
| LP8 | 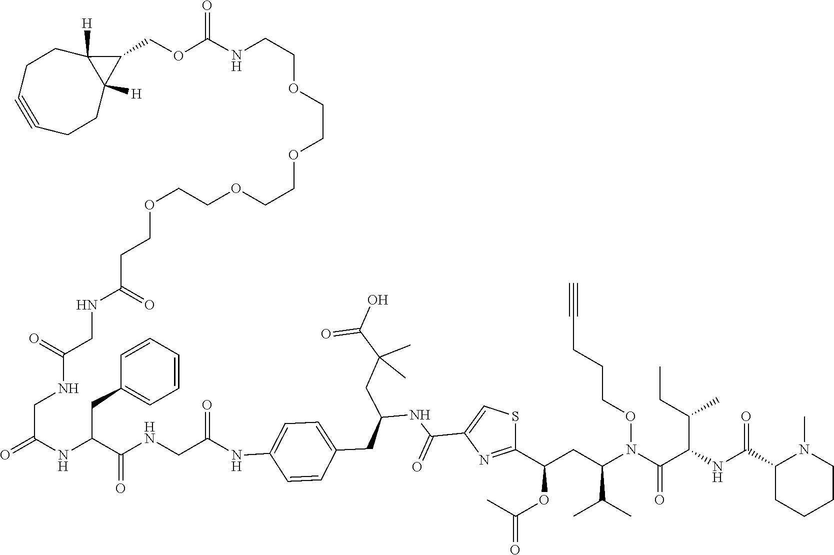 | BCN-PEG₄- GGFG (SEQ ID NO: 23) | P34 |
| LP9 | 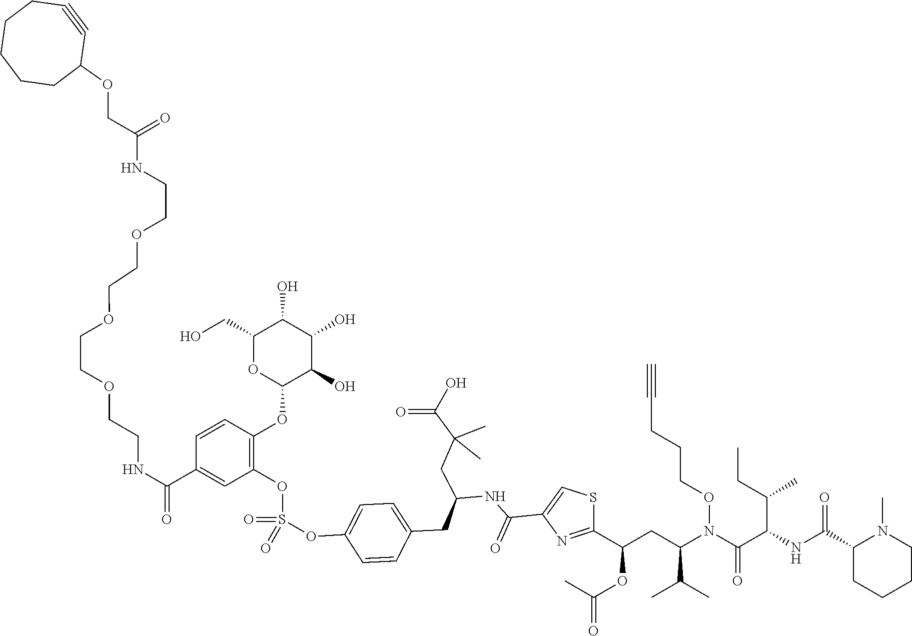 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(OS(=O)(=O)Oc3cc(C(=O)NCCOCCOCCOCCNC(=O)COC4C#CCCCCC4)ccc3O[C@@H]3O[C@H](CO)[C@H](O)[C@H](O)[C@H]3O)cc2)CC(C)(C)C(=O)O)cs1)C(C)C] | COT-PEG₄- HOPAS | P51 |
| LP10 | 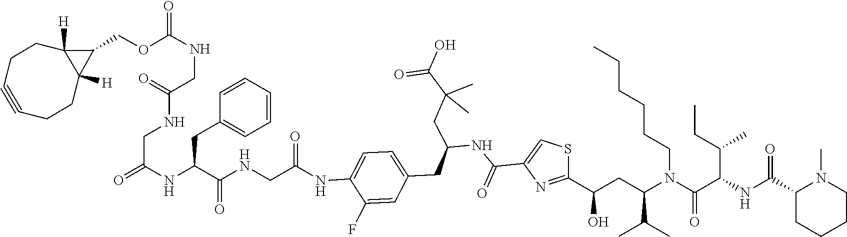 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(NC(=O)CNC(=O)[C@H](Cc3ccccc3)NC(=O)CNC(=O)CNC(=O)OC[C@@H]3[C@@H]4CCC#CCC[C@@H]43)c(F)c2)CC(C)(C)C(=O)O)cs1)C(C)C] | BCN-GGFG (SEQ ID NO: 23) | P1 |
| LP11 | 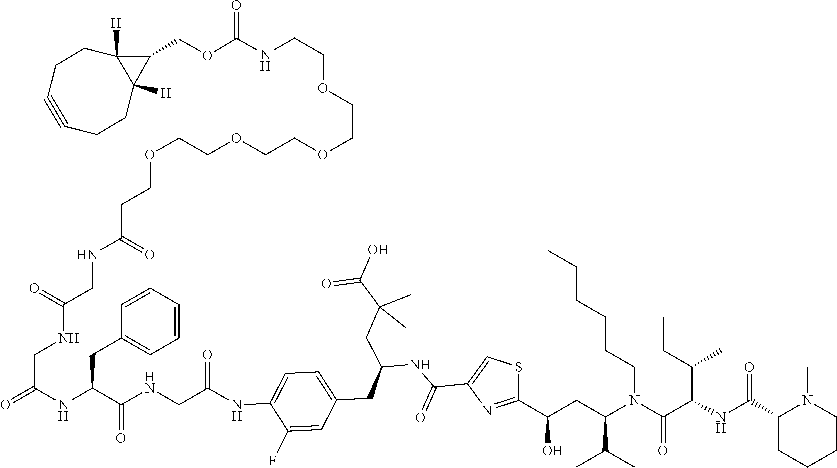 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(NC(=O)CNC(=O)[C@H](Cc3ccccc3)NC(=O)CNC(=O)CNC(=O)CCOCCOCCOCCOCCNC(=O)OC[C@@H]3[C@@H]4CCC#CCC[C@@H]43)c(F)c2)CC(C)(C)C(=O)O)cs1)C(C)C] | BCN-PEG₄- GGFG (SEQ ID NO: 23) | P1 |
| LP12 | 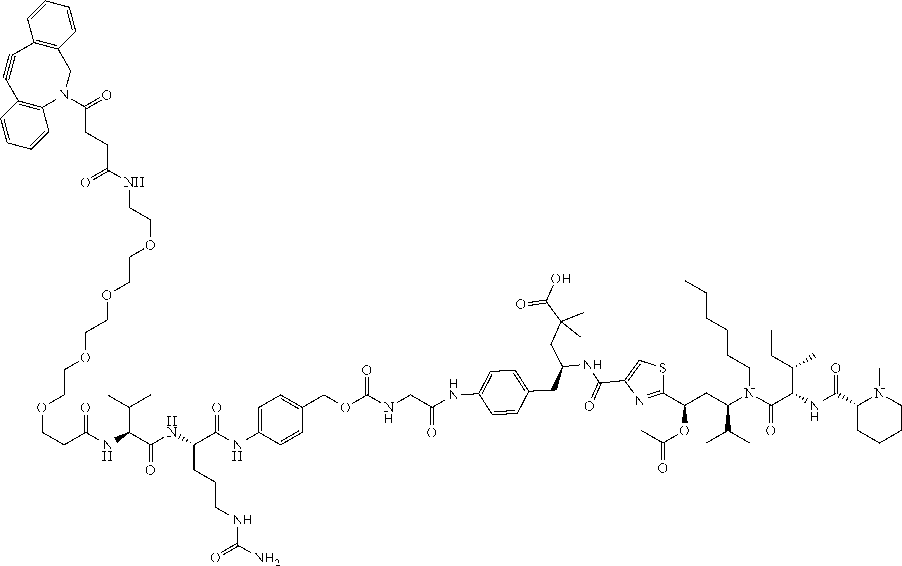 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CNC(=O)OCc3ccc(NC(=O)[C@H](CCCNC(N)=O)NC(=O)[C@@H](NC(=O)CCOCCOCCOCCOCCNC(=O)CCC(=O)N4Cc5ccccc5C#Cc5ccccc54)C(C)C)cc3)cc2)CC(C)(C)C(=O)O)cs1)C(C)C] | DIBAC- PEG₄- vcPAB | P28 |
| LP13 | 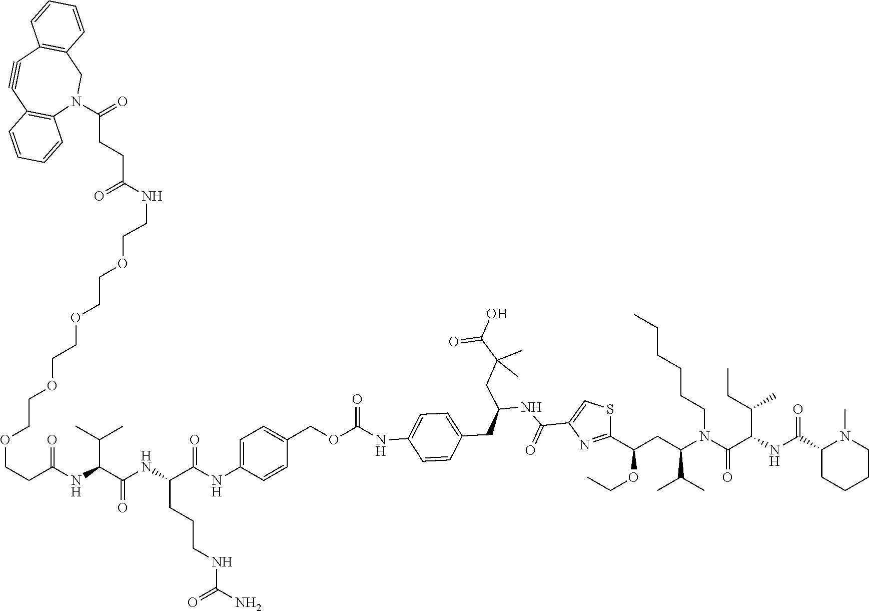 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)OCc3ccc(NC(=O)[C@H](CCCNC(N)=O)NC(=O)[C@@H](NC(=O)CCOCCOCCOCCOCCNC(=O)CCC(=O)N4Cc5ccccc5C#Cc5ccccc54)C(C)C)cc3)cc2)CC(C)(C)C(=O)O)cs1)C(C)C] | DIBAC- PEG₄- vcPAB | P8 |
| LP14 | 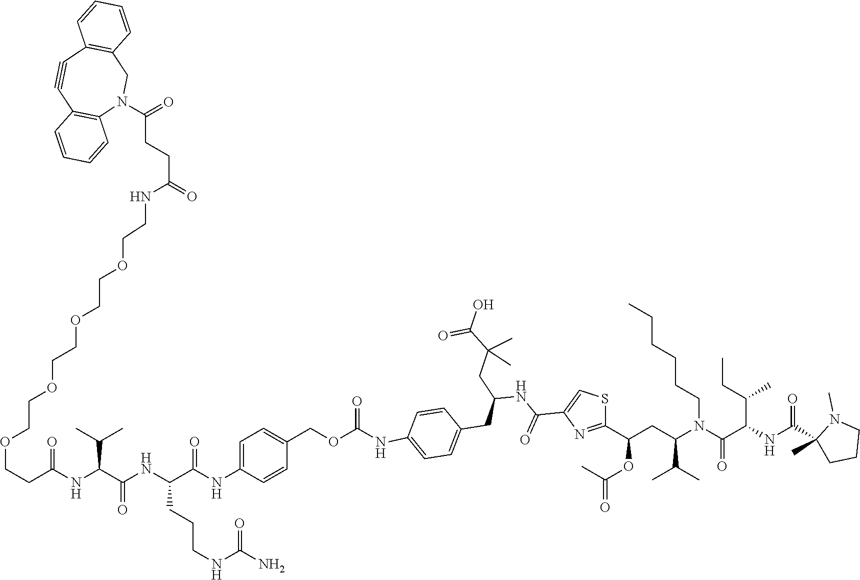 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(NC(=O)OCc3ccc(NC(=O)[C@H](CCCNC(N)=O)NC(=O)[C@@H](NC(=O)CCOCCOCCOCCOCCNC(=O)CCC(=O)N4Cc5ccccc5C#Cc5ccccc54)C(C)C)cc3)cc2)CC(C)(C)C(=O)O)cs1)C(C)C] | DIBAC- PEG₄- vcPAB | P19 |
| LP15 | 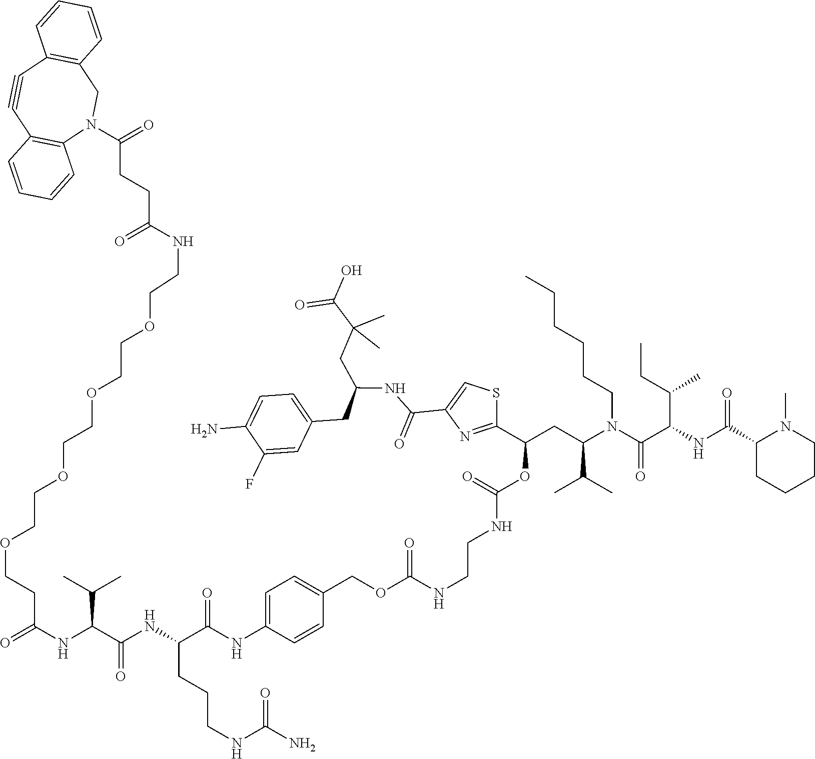 [SMILES: CCCCCCN(C(=O)[C@@H](NC(=O)[C@H]1CCCCN1C)[C@@H](C)CC)[C@H](C[C@@H](OC(=O)NCCNC(=O)OCc1ccc(NC(=O)[C@H](CCCNC(N)=O)NC(=O)[C@@H](NC(=O)CCOCCOCCOCCOCCNC(=O)CCC(=O)N2Cc3ccccc3C#Cc3ccccc32)C(C)C)cc1)c1nc(C(=O)N[C@@H](Cc2ccc(N)c(F)c2)CC(C)(C)C(=O)O)cs1)C(C)C] | DIBAC- PEG₄- vcPAB | P5 |
| LP16 | 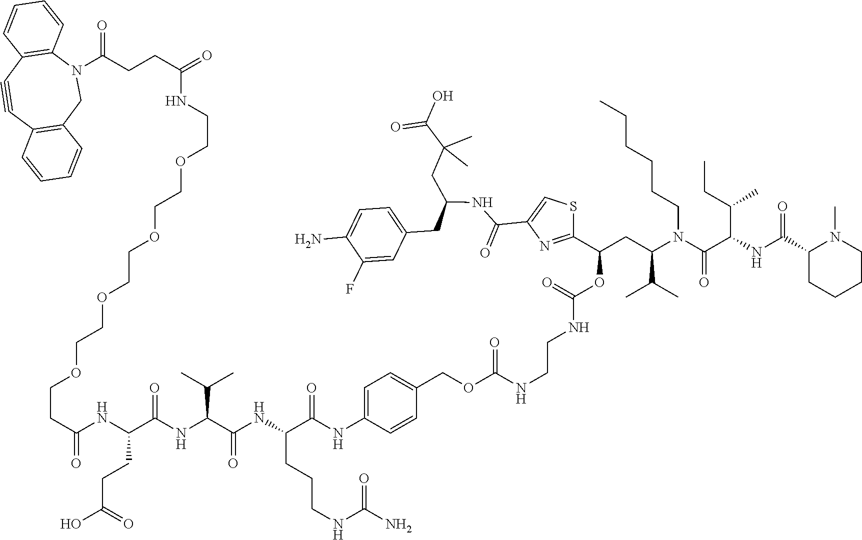 [SMILES: CCCCCCN(C(=O)[C@@H](NC(=O)[C@H]1CCCCN1C)[C@@H](C)CC)[C@H](C[C@@H](OC(=O)NCCNC(=O)OCc1ccc(NC(=O)[C@H](CCCNC(N)=O)NC(=O)[C@@H](NC(=O)[C@H](CCC(=O)O)NC(=O)CCOCCOCCOCCOCCNC(=O)CCC(=O)N2Cc3ccccc3C#Cc3ccccc32)C(C)C)cc1)c1nc(C(=O)N[C@@H](Cc2ccc(N)c(F)c2)CC(C)(C)C(=O)O)cs1)C(C)C] | DIBAC- PEG₄- EvcPAB | P5 |
| LP17 | 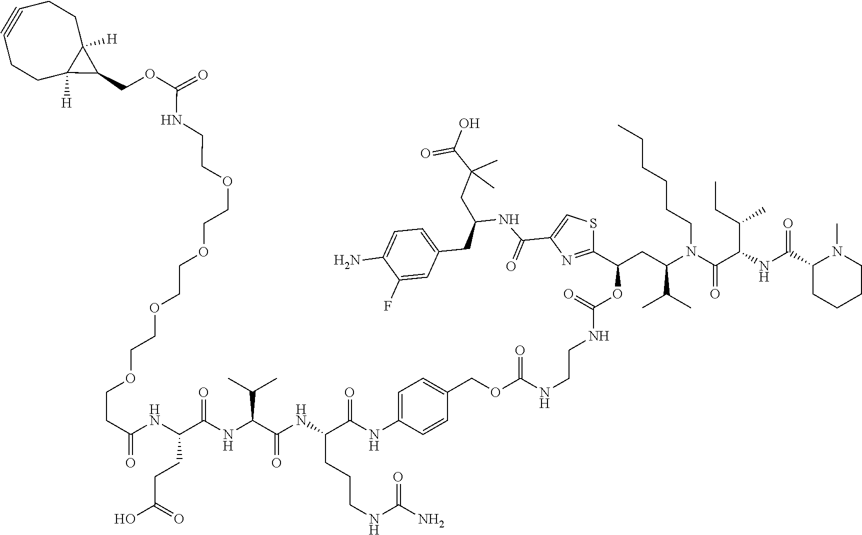 [SMILES: CCCCCCN(C(=O)[C@@H](NC(=O)[C@H]1CCCCN1C)[C@@H](C)CC)[C@H](C[C@@H](OC(=O)NCCNC(=O)OCc1ccc(NC(=O)[C@H](CCCNC(N)=O)NC(=O)[C@@H](NC(=O)[C@H](CCC(=O)O)NC(=O)CCOCCOCCOCCOCCNC(=O)OC[C@@H]2[C@@H]3CCC#CCC[C@@H]32)C(C)C)cc1)c1nc(C(=O)N[C@@H](Cc2ccc(N)c(F)c2)CC(C)(C)C(=O)O)cs1)C(C)C] | BCN-PEG₄- EvcPAB | P5 |
| LP18 | 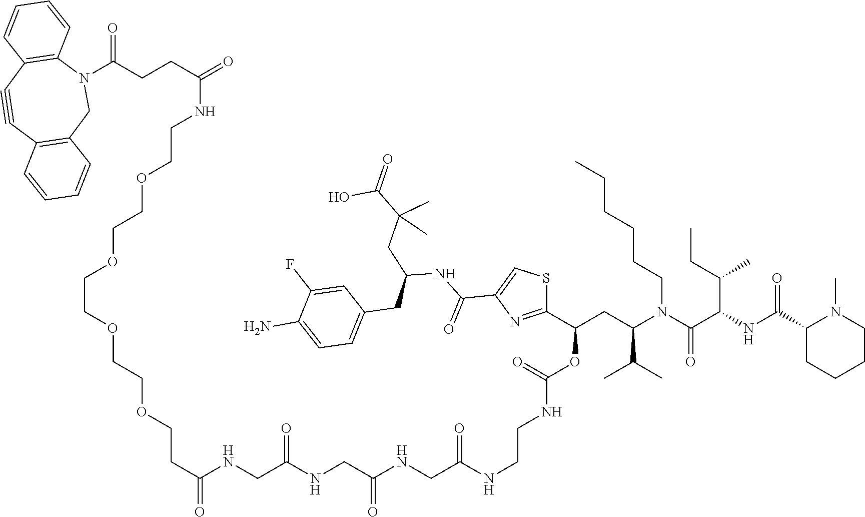 [SMILES: CCCCCCN(C(=O)[C@@H](NC(=O)[C@H]1CCCCN1C)[C@@H](C)CC)[C@H](C[C@@H](OC(=O)NCCNC(=O)CNC(=O)CNC(=O)CNC(=O)CCOCCOCCOCCOCCNC(=O)CCC(=O)N1Cc2ccccc2C#Cc2ccccc21)c1nc(C(=O)N[C@@H](Cc2ccc(N)c(F)c2)CC(C)(C)C(=O)O)cs1)C(C)C] | DIBAC- PEG₄-GGG | P5 |
| LP19 | 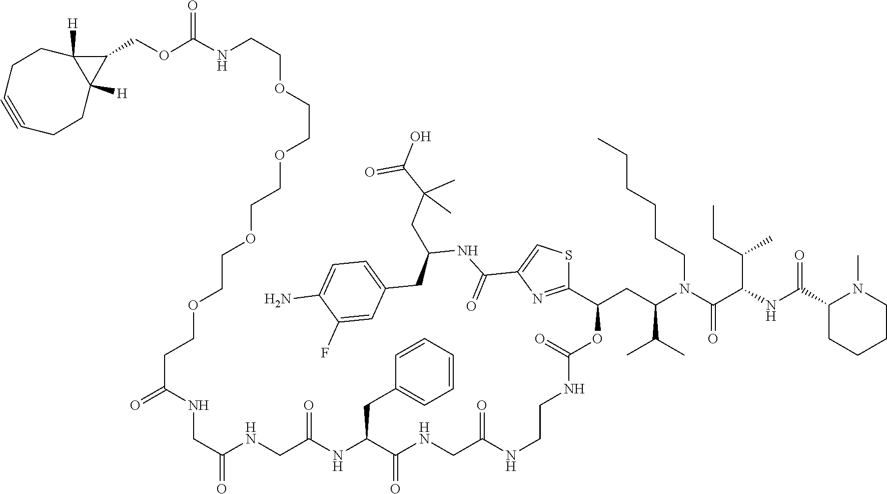 [SMILES: CCCCCCN(C(=O)[C@@H](NC(=O)[C@H]1CCCCN1C)[C@@H](C)CC)[C@H](C[C@@H](OC(=O)NCCNC(=O)CNC(=O)[C@H](Cc1ccccc1)NC(=O)CNC(=O)CNC(=O)CCOCCOCCOCCOCCNC(=O)OC[C@@H]1[C@@H]2CCC#CCC[C@@H]21)c1nc(C(=O)N[C@@H](Cc2ccc(N)c(F)c2)CC(C)(C)C(=O)O)cs1)C(C)C] | BCN-PEG₄- GGFG (SEQ ID NO: 23) | P5 |
| LP20 | 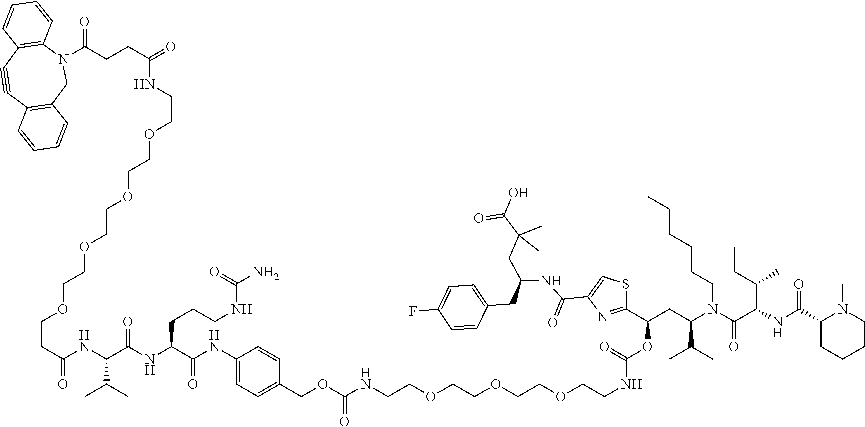 [SMILES: CCCCCCN(C(=O)[C@@H](NC(=O)[C@H]1CCCCN1C)[C@@H](C)CC)[C@H](C[C@@H](OC(=O)NCCOCCOCCOCCNC(=O)OCc1ccc(NC(=O)[C@H](CCCNC(N)=O)NC(=O)[C@@H](NC(=O)CCOCCOCCOCCOCCNC(=O)CCC(=O)N2Cc3ccccc3C#Cc3ccccc32)C(C)C)cc1)c1nc(C(=O)N[C@@H](Cc2ccc(F)cc2)CC(C)(C)C(=O)O)cs1)C(C)C] | DIBAC- PEG₄- vcPAB | P11 |
| LP21 | 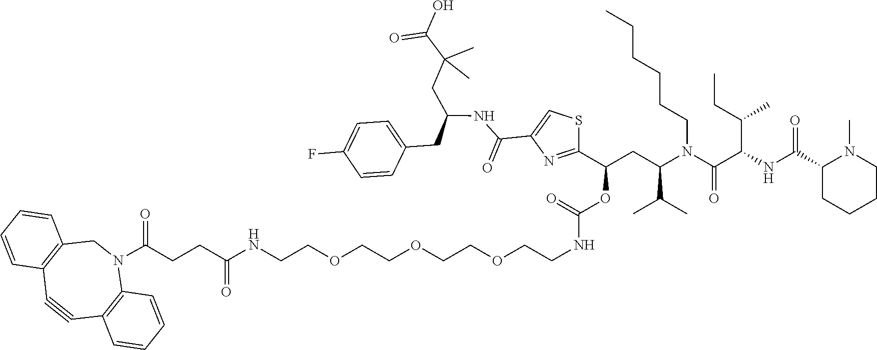 [SMILES: CCCCCCN(C(=O)[C@@H](NC(=O)[C@H]1CCCCN1C)[C@@H](C)CC)[C@H](C[C@@H](OC(=O)NCCOCCOCCOCCNC(=O)CCC(=O)N1Cc2ccccc2C#Cc2ccccc21)c1nc(C(=O)N[C@@H](Cc2ccc(F)cc2)CC(C)(C)C(=O)O)cs1)C(C)C] | DIBAC | P11 |
| LP22 | 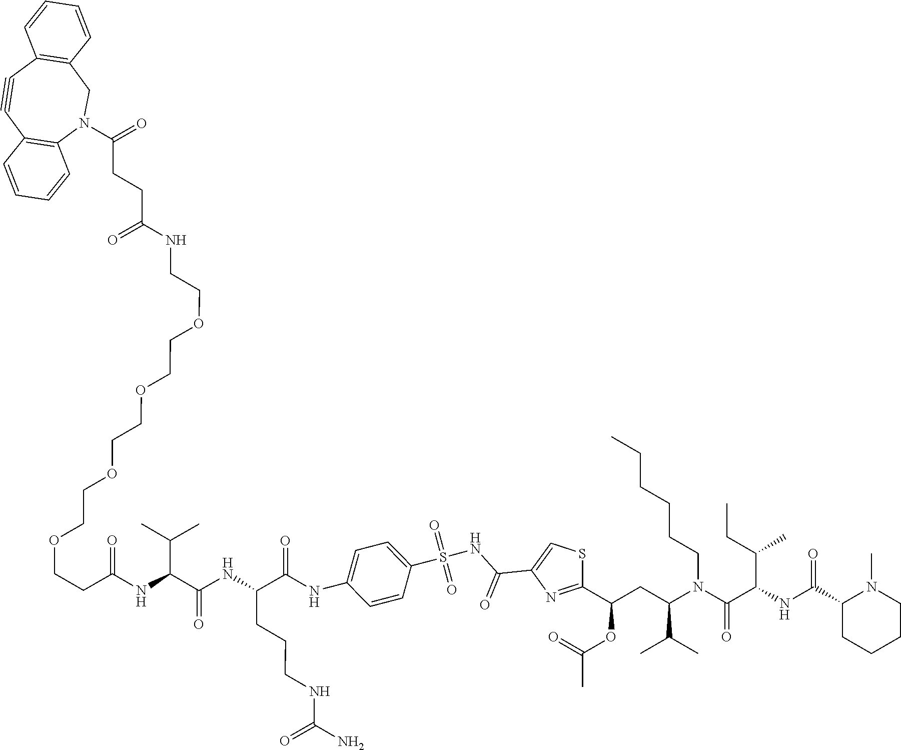 [SMILES: CCCCCCN(C(=O)[C@@H](NC(=O)[C@H]1CCCCN1C)[C@@H](C)CC)[C@H](C[C@@H](OC(C)=O)c1nc(C(=O)NS(=O)(=O)c2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)cs1)C(C)C] | DIBAC- PEG₄-vc | P43 |
| LP23 | 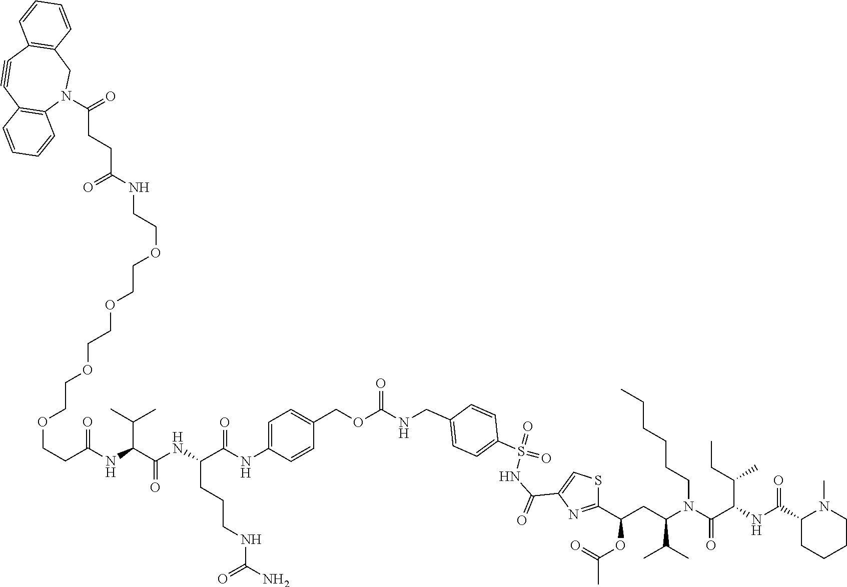 [SMILES: CCCCCCN(C(=O)[C@@H](NC(=O)[C@H]1CCCCN1C)[C@@H](C)CC)[C@H](C[C@@H](OC(C)=O)c1nc(C(=O)NS(=O)(=O)c2ccc(CNC(=O)OCc3ccc(NC(=O)[C@H](CCCNC(N)=O)NC(=O)[C@@H](NC(=O)CCOCCOCCOCCOCCNC(=O)CCC(=O)N4Cc5ccccc5C#Cc5ccccc54)C(C)C)cc3)cc2)cs1)C(C)C] | DIBAC- PEG₄- vcPAB | P42 |
| LP24 | 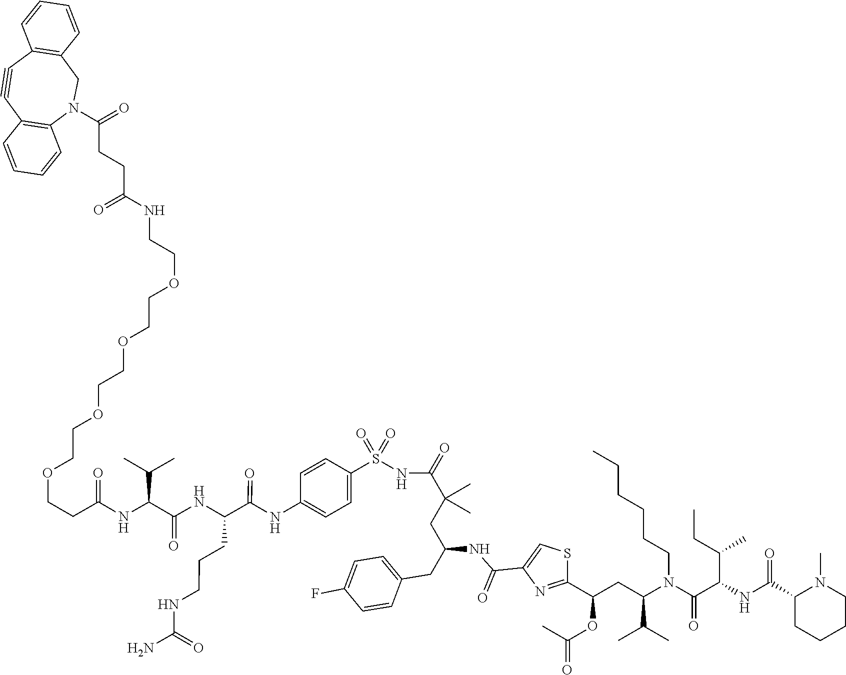 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)NS(=O)(=O)c2ccc(NC(=O)[C@H](CCCNC(N)=O)NC(=O)[C@@H](NC(=O)CCOCCOCCOCCOCCNC(=O)CCC(=O)N3Cc4ccccc4C#Cc4ccccc43)C(C)C)cc2)cs1)C(C)C] | DIBAC- PEG₄-vc | P47 |
| LP25 | 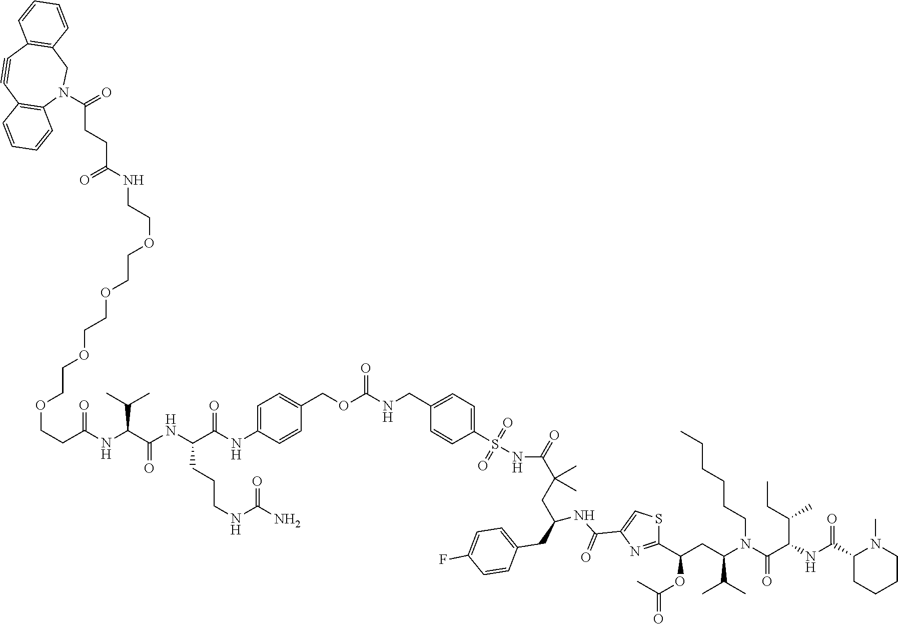 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)NS(=O)(=O)c2ccc(CNC(=O)OCc3ccc(NC(=O)[C@H](CCCNC(N)=O)NC(=O)[C@@H](NC(=O)CCOCCOCCOCCOCCNC(=O)CCC(=O)N4Cc5ccccc5C#Cc5ccccc54)C(C)C)cc3)cc2)cs1)C(C)C] | DIBAC- PEG₄- vcPAB | P46 |
| LP26 | 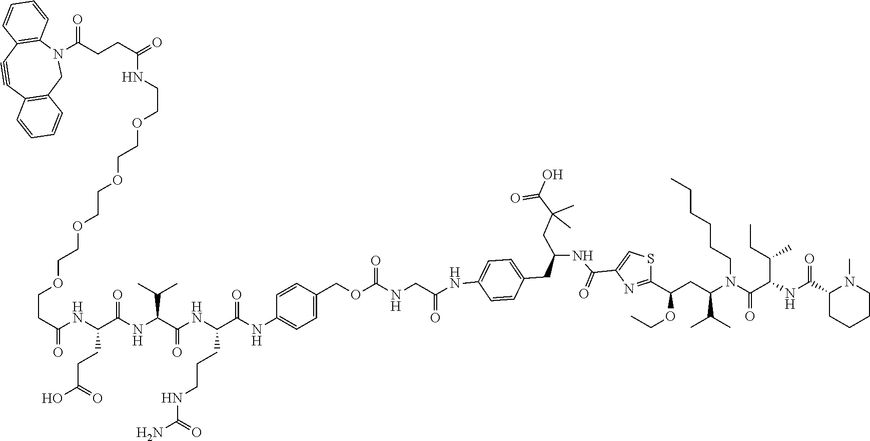 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CNC(=O)OCc3ccc(NC(=O)[C@H](CCCNC(N)=O)NC(=O)[C@@H](NC(=O)[C@H](CCC(=O)O)NC(=O)CCOCCOCCOCCOCCNC(=O)CCC(=O)N4Cc5ccccc5C#Cc5ccccc54)C(C)C)cc3)cc2)CC(C)(C)C(=O)O)cs1)C(C)C] | DIBAC- PEG₄- EvcPAB- Gly | P8 |

TABLE 8
Chemical Properties of Tubulysin Linker-payloads.

| | | | | | | HPLC | HPLC |
|---|---|---|---|---|---|---|---|
| | | | | | | purity | RT |
| # | Linker-payloads | cLogP | MF | MW | Mass m/z | (%) | (min) |
| LP2 | NH₂-PEG₄-Evc-P34 | −1.98 | C₇₀H₁₁₂N₁₂O₁₉S | 1457.8 | 729 | 99 | 6.63 |
| | | | | | (M/2 + H) | | (B) |
| LP3 | BCN-PEG₄-Evc-P34 | 2.62 | C₈₁H₁₂₄N₁₂O₂₁S | 1634.0 | 817 | 99 | 7.06 |
| | | | | | (M/2 + H) | | (B) |
| LP4 | BCN-PEG₄-EvcPAB-P34 | 4.19 | C₈₉H₁₃₁N₁₃O₂₃S | 1783.2 | 893 | 99 | 6.95 |
| | | | | | (M/2 + H) | | (A) |
| LP5 | COT-GGG-P34 | 2.22 | C₅₉H₈₅N₉O₁₃S | 1160.4 | 1161 | 99 | 7.76 |
| | | | | | (M + H) | | (B) |
| LP6 | BCN-GGGG-P34 | 1.74 | C₆₂H₈₈N₁₀O₁₄S | 1229.5 | 615 | 99 | 7.51 |
| | (SEQ ID NO: 22) | | | | (M/2 + H) | | (B) |
| LP7 | DIBAC-PEG₄-GGFG-P34 | 3.03 | C₈₈H₁₁₆N₁₂O₁₉S | 1678.0 | 839 | 99 | 7.94 |
| | (SEQ ID NO: 23) | | | | (M/2 + H) | | (B) |
| LP8 | BCN-PEG₄-GGFG-P34 | 2.91 | C₈₀H₁₁₅N₁₁O₁₉S | 1566.9 | 784 | 99 | 7.34 |
| | (SEQ ID NO: 23) | | | | (M/2 + H) | | (B) |
| LP9 | COT-PEG₃-HOPAS-P51 | 2.92 | C₇₄H₁₀₇N₇O₂₄S₂ | 1542.8 | 772 | 99 | 8.01 |
| | | | | | (M/2 + H) | | (B) |
| LP10 | BCN-GGFG-P1 | 4.77 | C₆₈H₉₇FN₁₀O₁₂S | 1297.6 | 649 | 99 | 8.56 |
| | (SEQ ID NO: 23) | | | | (M/2 + H) | | (B) |
| LP11 | BCN-PEG₄-GGFG-P1 | 3.71 | C₇₉H₁₁₈FN₁₁O₁₇S | 1544.9 | 773 | 97 | 7.20 |
| | (SEQ ID NO: 23) | | | | (M/2 + H) | | (A) |
| LP12 | DIBAC-PEG₄-vcPAB-G-P7 | 5.52 | C₉₅H₁₃₄N₁₄O₂₀S | 1824.3 | 913 | 99 | 8.13 |
| | | | | | (M/2 + H) | | (B) |
| LP13 | DIBAC-PEG₄-vcPAB-P8 | 7.19 | C₉₃H₁₃₃N₁₃O₁₈S | 1753.2 | 877 | 99 | 8.78 |
| | | | | | (M + H) | | (B) |
| LP14 | DIBAC-PEG₄-vcPAB-P19 | 6.57 | C₉₃H₁₃₁N₁₃O₁₉S | 1767.2 | 884 | 99 | 8.99 |
| | | | | | (M/2 + H) | | (B) |
| LP15 | DIBAC-PEG₄-vcPAB-P5 | 5.64 | C₉₄H₁₃₄FN₁₅O₁₉S | 1829.3 | 611 | 95 | 8.39 |
| | | | | | (M/3 + H); | | (B) |
| | | | | | 915 | | |
| | | | | | (M/2 + H) | | |
| LP16 | DIBAC-PEG₄-EvcPAB-P5 | 4.41 | C₉₉H₁₄₁FN₁₆O₂₂S | 1958.4 | 653 | 99 | 7.79 |
| | | | | | (M/3 + H); | | (B) |
| | | | | | 980 | | |
| | | | | | (M/2 + H) | | |
| LP17 | BCN-PEG₄-EvcPAB-P5 | 4.29 | C₉₁H₁₄₀FN₁₅O₂₂S | 1847.3 | 925 | 99 | 7.50 |
| | | | | | (M/2 + H) | | (B) |
| LP18 | DIBAC-PEG₄-GGG-P5 | 2.02 | C₈₁H₁₁₆FN₁₃O₁₇S | 1595.0 | 798 | 99 | 8.50 |
| | | | | | (M/2 + H) | | (B) |
| LP19 | BCN-PEG₄-GGFG-P5 | 3.02 | C₈₂H₁₂₄FN₁₃O₁₈S | 1631.0 | 816 | 99 | 7.72 |
| | (SEQ ID NO: 23) | | | | (M/2 + H) | | (B) |
| LP20 | DIBAC-PEG₄-vcPAB-P11 | 6.33 | C₁₀₀H₁₄₅FN₁₄O₂₂S | 1946.4 | 649 | 99 | 9.44 |
| | | | | | (M/3 + H); | | (B) |
| | | | | | 974 | | |
| | | | | | (M/2 + H) | | |
| LP21 | DIBAC-P11 | 4.41 | C₇₀H₉₇FN₈O₁₂S | 1293.7 | 647 | 95 | 11.75 |
| | | | | | (M/2 + H) | | (B) |
| LP22 | DIBAC-PEG₄-vc-P43 | 4.53 | C₇₈H₁₁₂N₁₂O₁₇S₂ | 1553.9 | 777 | 99 | 8.15 |
| | | | | | (M/2 + H) | | (B) |
| LP23 | DIBAC-PEG₄-vcPAB-P42 | 5.82 | C₈₇H₁₂₁N₁₃O₁₉S₂ | 1717.8 | 573 | 95 | 8.32 |
| | | | | | (M/3 + H); | | (B) |
| | | | | | 859 | | |
| | | | | | (M/2 + H) | | |
| LP24 | DIBAC-PEG₄-vc-P47 | 7.34 | C₉₁H₁₂₈FN₁₃O₁₈S₂ | 1775.2 | 888 | 99 | 8.76 |
| | | | | | (M/2 + H) | | (B) |
| LP25 | DIBAC-PEG₄-vcPAB-P46 | 8.63 | C₁₀₀H₁₃₇FN₁₄O₂₀S | 1938.4 | 647 | 99 | 8.41 |
| | | | | | (M/3 + H) | | (B) |
| LP26 | DIBAC-PEG₄-EvcPAB-Gly-P8 | 4.87 | C₁₀₀H₁₄₃N₁₅O₂₂S | 1939.4 | 647 | 99.9 | 7.35 |
| | | | | | (M/3 + H) | | (A) |

TABLE 9A
Linker-P34

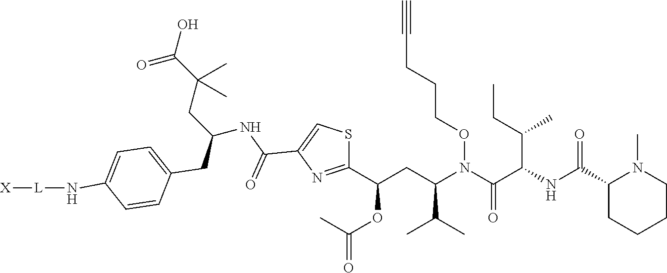

| Payload | X-L-P | |
|---|---|---|
| # | # | X-L- |
| P34 | LP2 | NH₂-PEG₄-Evc- |
| | LP3 | BCN-PEG₄-Evc- |
| | LP4 | BCN-PEG₄- |
| | | EvcPAB- |
| | LP5 | COT-GGG- |
| | LP6 | BCN-GGGG- |
| | | (SEQ ID NO: 22) |
| | LP7 | DIBAC-PEG₄- |
| | | GGFG- |
| | | (SEQ ID NO: 23) |
| | LP8 | BCN-PEG₄-GGFG- |
| | | (SEQ ID NO: 23) |

TABLE 9B
Linker-payloads via Tup-phenol

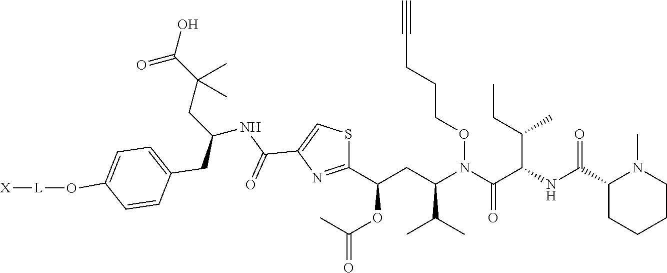

| Payload | X-L-P | |
|---|---|---|
| # | # | X-L- |
| P51 | LP9 | COT-PEG₃-HOPAS- |

TABLE 9C
Other Linker-payloads via Tup-aniline

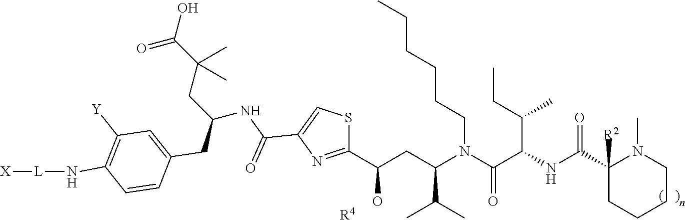

| Payload | | | | | X-L-P | |
|---|---|---|---|---|---|---|
| # | n | R² | R⁴ | Y | # | X-L- |
| P1 | 1 | H | H | F | LP10 | BCN-GGFG- |
| | | | | | | (SEQ ID NO: 23) |
| | | | | | LP11 | BCN-PEG₄- |
| | | | | | | GGFG- |
| | | | | | | (SEQ ID NO: 23) |
| P7 | 1 | H | Ac | H | LP12 | DIBAC-PEG₄- |
| | | | | | | vcPAB-G- |
| P8 | 1 | H | Et | H | LP13 | DIBAC-PEG₄- |
| | | | | | | vcPAB- |
| | | | | | LP26 | DIBAC-PEG₄- |
| | | | | | | EvcPAB-Gly-P8 |
| P19 | 0 | Me | Ac | H | LP14 | DIBAC-PEG₄- |
| | | | | | | vcPAB- |

TABLE 9D
Linker-carbamate-Tub

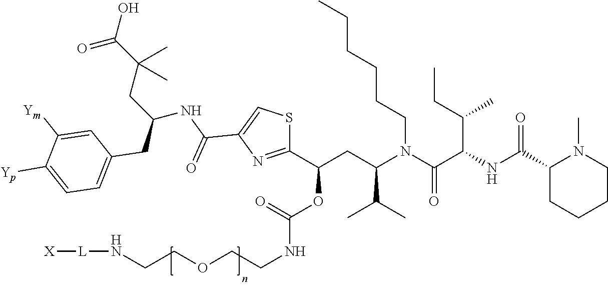

| Payload | | | | X-L-P | |
|---|---|---|---|---|---|
| # | n | Yₚ | Yₘ | # | X-L- |
| P5 | 0 | NH2 | F | LP15 | DIBAC-PEG₄- |
| | | | | | vcPAB- |
| | | | | LP16 | DIBAC-PEG₄- |
| | | | | | EvcPAB- |
| | | | | LP17 | BCN-PEG₄- |
| | | | | | EvcPAB- |
| | | | | LP18 | DIBAC-PEG₄- |
| | | | | | GGG- |
| | | | | LP19 | BCN-PEG₄- |
| | | | | | GGFG |
| | | | | | (SEQ ID NO: 23) |
| P11 | 3 | F | H | LP20 | DIBAC-PEG₄- |
| | | | | | vcPAB- |
| | | | | LP21 | DIBAC- |

TABLE 9E
Linker-N-acylsulfonamide-Tub

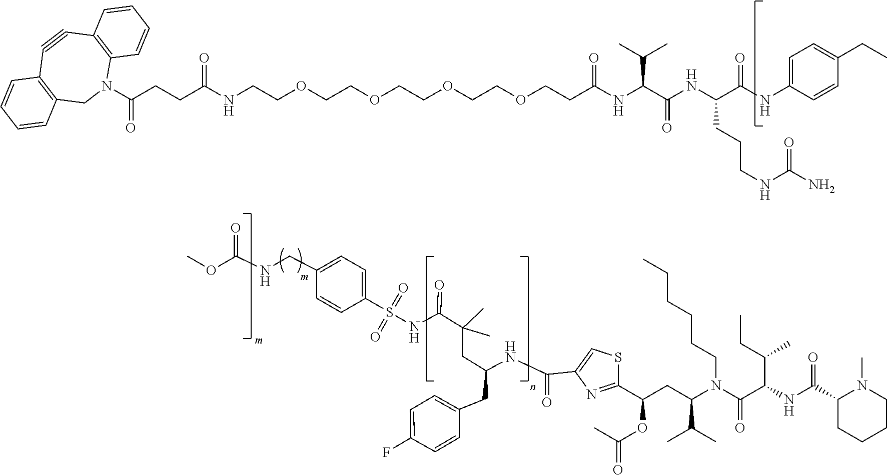

| Payload | X-L-P | | | |
|---|---|---|---|---|
| # | # | X-L | m | n |
| P43 | LP22 | DIBAC-PEG₄-vc- | 0 | 0 |
| P42 | LP23 | DIBAC-PEG₄-vcPAB- | 1 | 0 |
| P47 | LP24 | DIBAC-PEG₄-vc- | 0 | 1 |
| P46 | LP25 | DIBAC-PEG₄-vcPAB- | 1 | 1 |

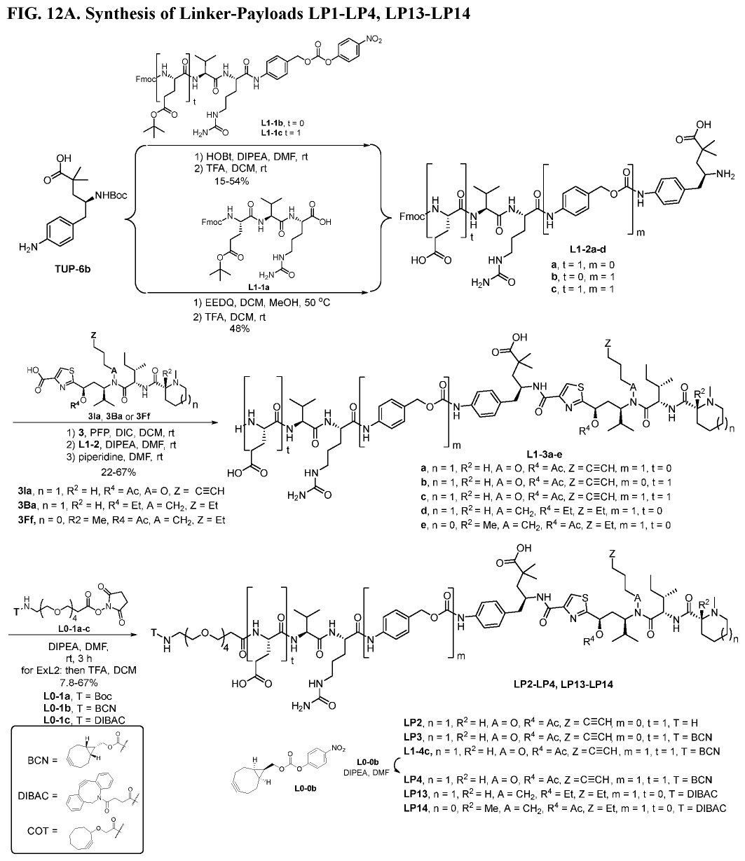

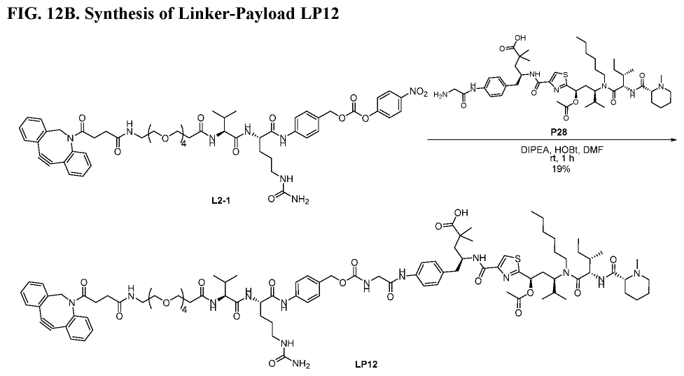

Synthesis of vcPAB-Linker-payloads LP2-LP4 and LP13-LP14 as in FIG. 12A.

### (2S)-2-[(2S)-2-[(2S)-5-(tert-Butoxy)-2-{[(9H-fluoren-9-ylmethoxy)carbonyl]amino}-5-oxopentanamido]-3-methylbutanamido]-5-(carbamoylamino)pentanoic Acid (L1-1a)

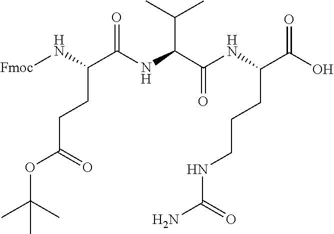

Following General Procedure IX using H-Val-Cit-OH (0.73 g, 2.1 mmol) with Fmoc-Glu(O^{t}Bu)-OSu (1.2 g, 2.3 mmol), provided Fmoc-Glu(O^{t}Bu)-Val-Cit-OH (L1-la) (0.60 g, 33% yield) as a white solid. ESI m/z: 682 (M+H)⁺.

### tert-Butyl (4S)-4-{[(1S)-1-{[(1S)-4-(carbamoylamino)-1-[(4-{[(4-nitrophenoxycarbonyl)oxy]methyl}phenyl)carbamoyl]butyl]carbamoyl}-2-methylpropyl]carbamoyl}-4-{[(9H-fluoren-9-ylmethoxy)carbonyl]amino}butanoate (L1-1c)

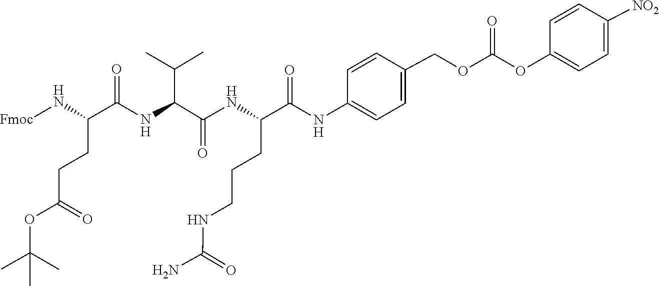

To a solution of Fmoc-Glu(O^{t}Bu)-OH (0.56 g, 1.3 mmol) in DMF (5 mL) were added HATU (0.50 g, 1.3 mmol) and DIPEA (0.34 g, 2.6 mmol). The reaction mixture was stirred at room temperature for 10 minutes before the addition of vcPAB (0.50 g, 1.3 mmol). The mixture was stirred at room temperature for an hour, and monitored by LCMS. The resulting mixture was purified by reversed phase flash chromatography (0-100% acetonitrile in aq. ammonium bicarbonate (10 mM)) to give Fmoc-Glu-Val-Cit-PAB (ESI m/z: 787 (M+H)⁺) as a white solid. Fmoc-Glu-Val-Cit-PAB was dissolved in DMF (5 mL). To the solution was added bis(4-nitrophenyl) carbonate (0.52 g, 1.7 mmol), DMAP (0.16 g, 1.3 mmol), and DIPEA (0.84 g, 6.5 mmol). The reaction mixture was stirred at room temperature for an hour, and monitored by LCMS. The resulting mixture was purified by reversed phase flash chromatography (0-100% acetonitrile in water) to give compound L1-1c (0.78 g, 63% yield) as a white solid. ESI m/z: 952 (M+H)⁺.

### (4S)-4-Amino-5-{4-[(2S)-5-(carbamoylamino)-2-[(2S)-2-[(2S)-4-carboxy-2-{[(9H-fluoren-9-ylmethoxy)carbonyl]amino}butanamido]-3-methylbutanamido]pentanamido]phenyl}-2,2-dimethylpentanoic acid (L1-2a)

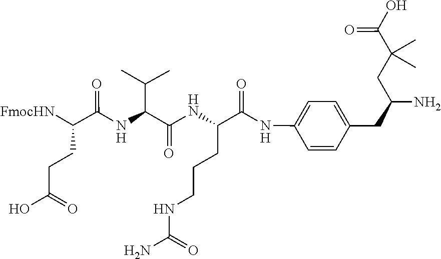

To a solution of Fmoc-Glu(O^{t}Bu)-Val-Cit-OH (L1-1a) (0.60 g, 0.88 mmol) in methanol (15 mL) was added EEDQ (0.23 g, 0.93 mmol) and TUP-6b (0.61 g, 1.8 mmol). The reaction mixture was stirred at 50° C. for 4 hours, and monitored by LCMS. The resulting mixture was filtered and the filtrate was concentrated in vacuo. The residue (0.80 g) was dissolved in DCM (9 mL). To the solution was added TFA (3 mL), and the mixture was stirred at room temperature for 2 hours until both Boc and ^{t}Bu were totally removed, according to LCMS. The resulting mixture was concentrated in vacuo and the residue was purified by reversed phase flash chromatography (0-40% acetonitrile in aq. ammonium bicarbonate (10 mM)) to give L1-2a (0.36 g, 48% yield from L1-1a) as a white solid. ESI m/z: 844 (M+H)⁺.

### (4S)-4-Amino-5-(4-{[({4-[(2S)-5-(carbamoylamino)-2-[(2S)-2-{[(9H-fluoren-9-ylmethoxy)carbonyl]amino}-3-methylbutanamido]pentanamido]phenyl}methoxy)carbonyl]amino}phenyl)-2,2-dimethylpentanoic Acid (L1-2b)

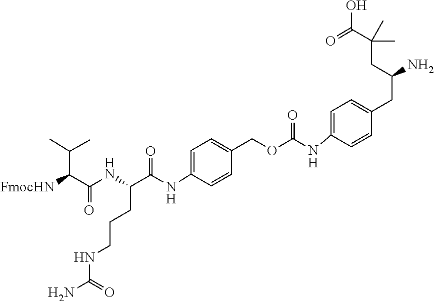

Following General Procedure X using Fmoc-vcPAB-PNP (L1-1b) (50 mg, 65 μmol) and amine TUP-6b (20 mg, 59 μmol) with HOBt, Boc-L1-2b (31 mg, ESI m/z 964 (M+H)⁺) was obtained as a white solid. Boc-L1-2b was dissolved in DCM (4 mL). To the solution was added TFA (0.5 mL), and the reaction mixture was stirred at room temperature for half an hour until Boc was totally removed, according to LCMS. The volatiles were removed in vacuo to give compound L1-2b (37 mg, 54% yield, TFA salt) as a brown oil. ESI m/z 433 (M/2+H)⁺.

### (4S)-4-Amino-5-(4-{[({4-[(2S)-5-(carbamoylamino)-2-[(2S)-2-[(2S)-4-carboxy-2-{[(9H-fluoren-9-ylmethoxy)carbonyl]amino}butanamido]-3-methylbutanamido]pentanamido]phenyl}methoxy)carbonyl]amino}phenyl)-2,2-dimethylpentanoic Acid (L1-2c)

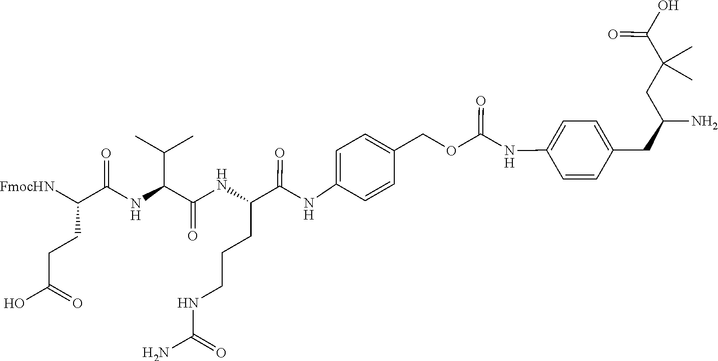

Following General Procedure X using Fmoc-Glu(O^{t}Bu)-Val-Cit-PAB-PNP (L1-1c) (0.10 g, 0.11 mmol) and amine TUP-6b with HOBt, Boc-L1-2c (ESI m/z: 1151 (M+H)⁺) was obtained as a white solid. Boc-L1-2c was dissolved in DCM (5 mL). To the solution was added TFA (1 mL), the reaction mixture was stirred at room temperature for an hour, and monitored by LCMS. The resulting mixture was concentrated in vacuo and the residue was purified by reversed phase flash chromatography (0-100% acetonitrile in aq. TFA (0.01%)) to give L1-2c (16 mg, 15% yield from L1-1c) as a white solid. ESI m/z: 994 (M+H)⁺.

### (4S)-4-({2-[(1R,3R)-1-(Acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-(4-{[({4-[(2S)-2-[(2S)-2-amino-3-methylbutanamido]-5-(carbamoylamino)pentanamido]phenyl}methoxy)carbonyl]amino}phenyl)-2,2-dimethylpentanoic Acid (L1-3a)

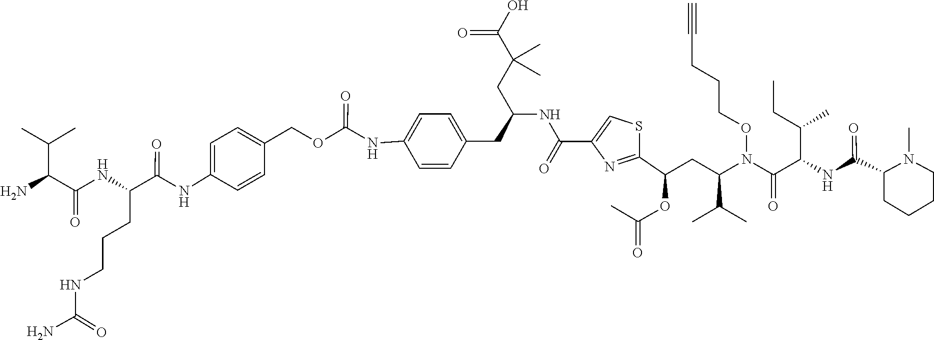

Following General Procedure VIII from L1-2b with 3Ia, compound L1-3a (17 mg, 67% yield from 3Ia) was obtained as a white solid. ESI m/z 615.8 (M/2+H)⁺.

### (4S)-4-({2-[(1R,3R)-1-(Acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-{4-[(2S)-2-[(2S)-2-[(2S)-2-amino-4-carboxybutanamido]-3-methylbutanamido]-5-(carbamoylamino)pentanamido]phenyl}-2,2-dimethylpentanoic Acid (L1-3b)

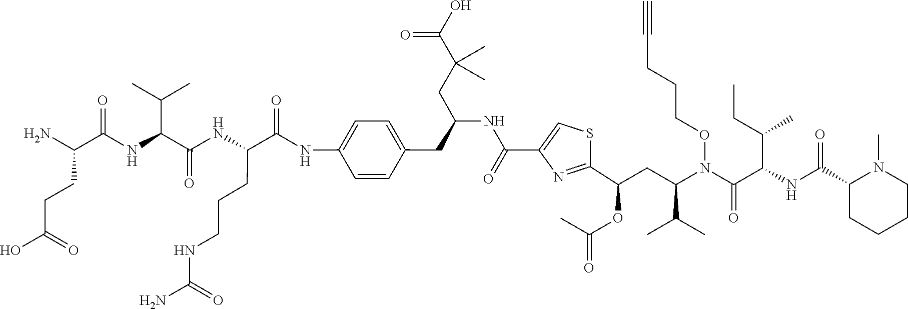 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)[C@H](CCCNC(N)=O)NC(=O)[C@@H](NC(=O)[C@@H](N)CCC(=O)O)C(C)C)cc2)CC(C)(C)C(=O)O)cs1)C(C)C]

Following General Procedure VIII from L1-2a with 3Ia (80 mg, 0.13 mmol), Fmoc-L1-3b (50 mg, ESI m/z: 717 (M/2+H)⁺) was obtained as a white solid after purification by reversed phase flash chromatography (0-100% acetonitrile in water). To a solution of Fmoc-L1-3b (16 mg) in DMF (1 mL) was added piperidine (4 mg, 47 μmol, excess), and the mixture was stirred at room temperature for 3 hours until Fmoc was totally removed according to LCMS. The resulting mixture was purified directly by reversed phase flash chromatography (0-70% acetonitrile in water) to give compound L1-3b (11 mg, 22% yield from 3Ia) as a white solid. ESI m/z 606 (M/2+H)⁺.

### (4S)-4-({2-[(1R,3R)-1-(Acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-(4-{[({4-[(2S)-2-[(2S)-2-[(2S)-2-amino-4-carboxybutanamido]-3-methylbutanamido]-5-(carbamoylamino)pentanamido]phenyl}methoxy)carbonyl]amino}phenyl)-2,2-dimethylpentanoic Acid (L1-3c)

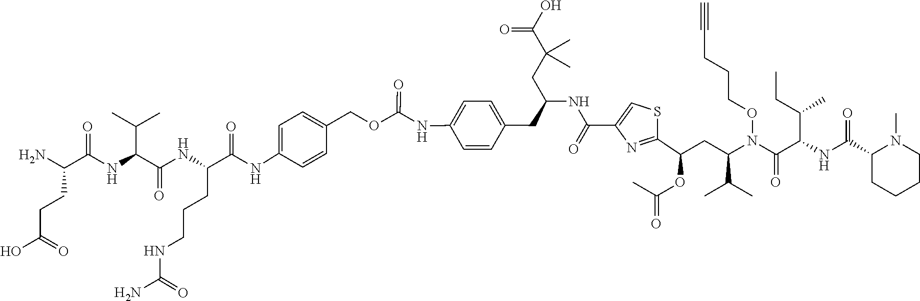 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)OCc3ccc(NC(=O)[C@H](CCCNC(N)=O)NC(=O)[C@@H](NC(=O)[C@@H](N)CCC(=O)O)C(C)C)cc3)cc2)CC(C)(C)C(=O)O)cs1)C(C)C]

Following General Procedure VIII from L1-2c with 3Ia, compound L1-3c (75 mg, 50% yield from 3Ia) was obtained as a white solid. ESI m/z 680.5 (M/2+H)⁺.

### (4S)-5-(4-{[({4-[(2S)-2-[(2S)-2-Amino-3-methylbutanamido]-5-(carbamoylamino)pentanamido]phenyl}methoxy)carbonyl]amino}phenyl)-4-({2-[(1R,3R)-1-ethoxy-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic Acid (L1-3d)

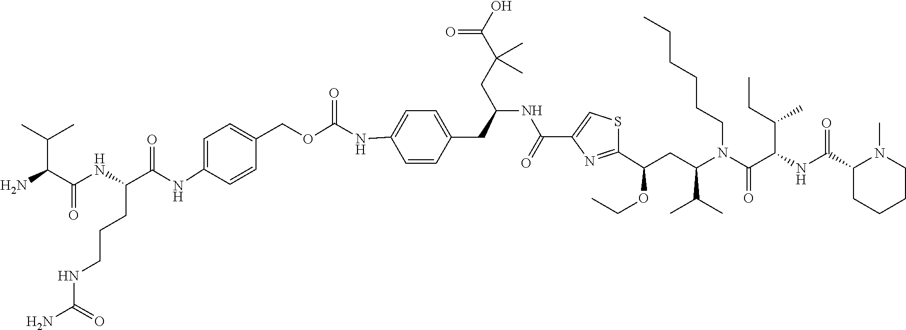 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)OCc3ccc(NC(=O)[C@H](CCCNC(N)=O)NC(=O)[C@@H](N)C(C)C)cc3)cc2)CC(C)(C)C(=O)O)cs1)C(C)C]

Following General Procedure VIII from L1-2b with 3Ba, compound L1-3d (17 mg, 66% yield from 3Ba) was obtained as a white solid. ESI m/z 610 (M/2+H)⁺.

### (4S)-4-({2-[(1R,3R)-1-(Acetyloxy)-3-[(2S,3S)-2-{[(2R)-1,2-dimethylpyrrolidin-2-yl]formamido}-N-hexyl-3-methylpentanamido]-4-methylpentyl]-1,3-thiazol-4-yl}formamido)-5-(4-{[({4-[(2S)-2-[(2S)-2-amino-3-methylbutanamido]-5-(carbamoylamino)pentanamido]phenyl}methoxy)carbonyl]amino}phenyl)-2,2-dimethylpentanoic Acid (L1-3e)

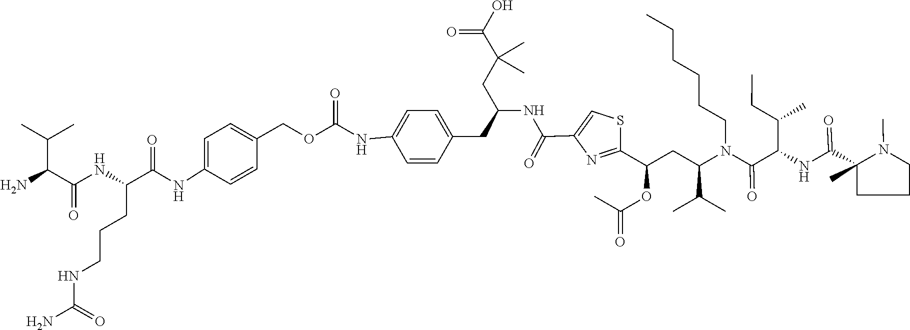 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(NC(=O)OCc3ccc(NC(=O)[C@H](CCCNC(N)=O)NC(=O)[C@@H](N)C(C)C)cc3)cc2)CC(C)(C)C(=O)O)cs1)C(C)C]

Following General Procedure VIII from L1-2b with 3Ff, compound L1-3e (20 mg, 37% yield from 3Ff) was obtained as a white solid. ESI m/z 617 (M/2+H)⁺.

### LP2: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-{4-[(2S)-2-[(2S)-2-[(2S)-2-(1-amino-3,6,9,12-tetraoxapentadecan-15-amido)-4-carboxybutanamido]-3-methylbutanamido]-5-(carbamoylamino)pentanamido]phenyl}-2,2-dimethylpentanoic Acid (LP2)

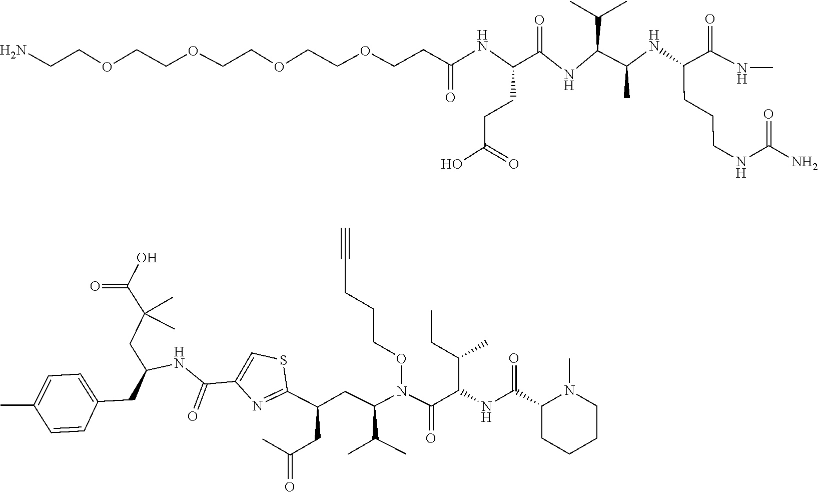

Following General Procedure IX from amine L1-3b (28 mg, 23 μmol) and OSu ester L0-1a, Boc-LP2 (26 mg) was obtained as a white solid. Boc-LP2 was dissolved in DCM (4 mL). To the solution was added TFA (1 mL), and the reaction mixture was stirred at room temperature for 4 hours until Boc was totally removed according to LCMS. The resulting mixture was concentrated in vacuo and the residue was purified by prep-HPLC (10-95% acetonitrile in aq. formic acid (0.01%)) to give linker-payload LP2 (11 mg, 33% yield from L1-3b) as a white solid. ESI m/z 729 (M/2+H)⁺. ¹H NMR (400 MHz, DMSO_{d6}) δ 9.90 (s, 1H), 8.46-8.42 (m, 1H), 8.36-8.30 (m, 1H), 8.18-8.16 (m, 1H), 7.89-7.79 (m, 2H), 7.61 (d, J=9.6 Hz, 2H), 7.48 (d, J=8.0 Hz, 2H), 7.10 (d, J=8.0 Hz, 2H), 6.31 (s, 1H), 5.82 (d, J=10.4 Hz, 1H), 5.55 (s, 1H), 4.76 (t, J=8.0 Hz, 1H), 4.34-4.30 (m, 1H), 4.28-4.24 (m, 2H), 4.19-4.16 (m, 1H), 4.09-4.03 (m, 2H), 3.65-3.61 (m, 2H), 3.59-3.53 (m, 9H), 3.52-3.47 (m, 10H), 2.99-2.94 (m, 2H), 2.89 (t, J=5.2 Hz, 2H), 2.87-2.83 (m, 2H), 2.80-2.76 (m, 1H), 2.70-2.66 (m, 1H), 2.43-2.41 (m, 1H), 2.38-2.32 (m, 3H), 2.13 (s, 4H), 2.10 (s, 3H), 2.03-1.93 (m, 4H), 1.86-1.80 (m, 4H), 1.68-1.59 (m, 5H), 1.54-1.50 (m, 1H), 1.48-1.41 (m, 3H), 1.40-1.32 (m, 3H), 1.18-1.12 (m, 1H), 1.07-1.02 (m, 7H), 0.95 (d, J=6.4 Hz, 3H), 0.89-0.80 (m, 18H) ppm.

### LP3: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-{4-[(2S)-2-[(2S)-2-[(2S)-2-[1-({[endo-bicyclo[6.1.0]non-4-yn-9-ylmethoxy]carbonyl}amino)-3,6,9,12-tetraoxapentadecan-15-amido]-4-carboxybutanamido]-3-methylbutanamido]-5-(carbamoylamino)pentanamido]phenyl}-2,2-dimethylpentanoic Acid (LP3)

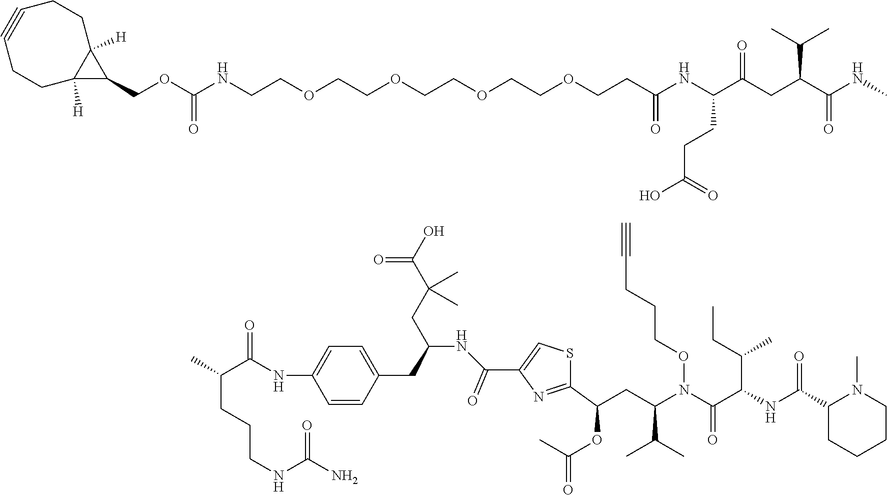

Following General Procedure IX from amine L1-3b (50 mg, 41 μmol) with OSu ester L0-1b, linker-payload LP3 (15 mg, 22% yield) was obtained as a white solid. ESI m/z: 817 (M/2+H)⁺. ¹H NMR (400 MHz, DMSO_{d6}) δ 9.91-9.89 (m, 1H), 8.17 (s, 2H), 8.08 (d, J=7.6 Hz, 1H), 7.79-7.70 (m, 2H), 7.70-7.60 (m, 1H), 7.47 (d, J=8.4 Hz, 2H), 7.13-7.08 (m, 3H), 6.00 (t, J=7.6 Hz, 1H), 5.82 (d, J=10.4 Hz, 1H), 5.43 (s, 2H), 4.76 (t, J=8.0 Hz, 1H), 4.38-4.31 (m, 2H), 4.30-4.22 (m, 2H), 4.21-4.16 (m, 1H), 4.08-4.00 (m, 4H), 3.61-3.55 (m, 2H), 3.51-3.46 (m, 13H), 3.41-3.36 (m, 4H), 3.14-3.09 (m, 2H), 3.06-2.99 (m, 1H), 2.96-2.90 (m, 1H), 2.86-2.84 (m, 1H), 2.82-2.76 (m, 1H), 2.70-2.64 (m, 1H), 2.44-2.40 (m, 1H), 2.39-2.30 (m, 5H), 2.26-2.20 (m, 4H), 2.18-2.10 (m, 11H), 2.03-1.92 (m, 4H), 1.86-18.0 (m, 3H), 1.70-1.62 (m, 4H), 1.56-1.50 (m, 3H), 1.44-1.35 (m, 4H), 1.29-1.23 (m, 1H), 1.09-1.02 (m, 7H), 0.96 (d, J=6.4 Hz, 3H), 0.90-0.79 (m, 20H) ppm.

### LP4: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-(4-{[({4-[(2S)-2-[(2S)-2-[(2S)-2-[1-({[endo-bicyclo[6.1.0]non-4-yn-9-ylmethoxy]carbonyl}amino)-3,6,9,12-tetraoxapentadecan-15-amido]-4-carboxybutanamido]-3-methylbutanamido]-5-(carbamoylamino)pentanamido]phenyl}methoxy)carbonyl]amino}phenyl)-2,2-dimethylpentanoic Acid (LP4)

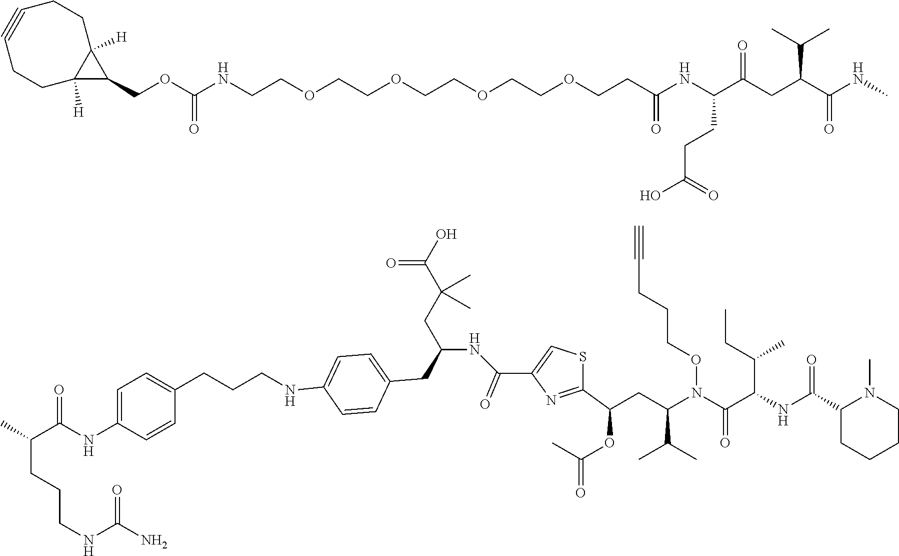

Following General Procedure IX from amine L1-3c and OSu ester L0-1a, Boc-L1-4c (35 mg, ESI m/z: 854 (M/2+H)⁺) was obtained as a white solid. Boc-L1-4c was dissolved in DCM (4 mL). To the solution was added TFA (1 mL), and the reaction mixture was stirred at room temperature for an hour until Boc was totally removed according to LCMS. The resulting mixture was concentrated in vacuo and the residue was purified by prep-HPLC (0-100% acetonitrile in aq. TFA (0.01%)) to give L1-4c (36 mg, ESI m/z: 804 (M/2+H)⁺) as a white solid. L1-4c was dissolved in DMF (3 mL). To the solution were added L0-Ob (9.0 mg, 29 μmol), HOBt (2.0 mg, 10 μmol) and DIPEA (5.0 mg, 39 μmol), the reaction mixture was stirred at room temperature overnight, and monitored by LCMS. The resulting mixture was directly purified by prep-HPLC (0-100% acetonitrile in aq. TFA (0.01%)) to give LP4 (4.0 mg, 7.8% yield from L1-3c) as a white solid. ESI m/z: 893 (M/2+H)⁺. ¹H NMR (400 MHz, DMSO_{d6}) δ 10.05 (s, 1H), 9.64 (s, 1H), 8.26 (s, 1H), 8.15 (s, 1H), 8.10 (d, J=7.7 Hz, 1H), 7.76 (d, J=8.2 Hz, 1H), 7.72 (d, J=9.0 Hz, 1H), 7.61 (m, 3H), 7.34 (m, 4H), 7.12-7.06 (m, 3H), 5.82 (d, J=11.4 Hz, 1H), 5.48 (s, 2H), 5.05 (s, 2H), 4.79-4.72 (m, 1H), 4.40-4.15 (m, 6H), 4.03 (m, 4H), 3.61-3.55 (m, 3H), 3.48 (d, J=5.5 Hz, 14H), 3.13-3.09 (m, 3H), 3.06-2.88 (m, 3H), 2.87-2.72 (m, 3H), 2.79-2.64 (m, 2H), 2.43-2.29 (m, 7H), 2.26-2.20 (m, 4H), 2.15-2.10 (m, 10H), 2.05-1.78 (m, 9H), 1.69-1.60 (m, 5H), 1.55-1.47 (m, 3H), 1.38-1.34 (m, 2H), 1.28-1.24 (m, 2H), 1.06 (s, 3H), 1.04 (s, 3H), 0.96 (d, J=6.5 Hz, 3H), 0.90-0.79 (m, 18H) ppm.

### LP13: (4S)-5-(4-{[({4-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methoxy)carbonyl]amino}phenyl)-4-({2-[(1R,3R)-1-ethoxy-4-methyl-3-[(2S,3S)-3-methyl-2-{[(2R)-1-methylpiperidin-2-yl]formamido}-N-(pentyloxy)pentanamido]pentyl]-1,3-thiazol-4-yl}formamido)-2,2-dimethylpentanoic Acid (LP13)

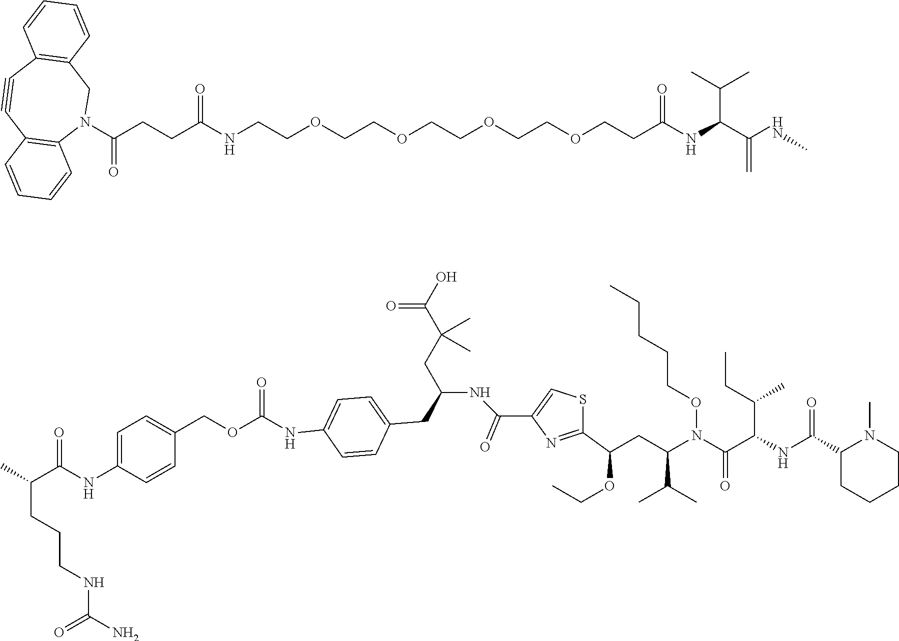

Following General Procedure IX from amine L1-3d and OSu ester L0-1c, linker-payload LP13 (24 mg, 33% yield) was obtained as a white solid. ESI m/z: 877 (M/2+H)⁺. ¹H NMR (500 MHz, DMSO_{d6}) δ 10.0 (s, 1H), 9.66 (s, 1H), 8.14 (s, 1H), 8.11 (d, J=7.5 Hz, 1H), 7.87 (d, J=9.0 Hz, 1H), 7.66 (t, J=5.5 Hz, 1H), 7.68-7.66 (m, 1H), 7.62-7.60 (m, 3H), 7.51-7.45 (m, 4H), 7.39-7.32 (m, 7H), 7.30-7.28 (m, 1H), 7.04 (d, J=8.5 Hz, 2H), 5.99 (t, J=6.0 Hz, 1H), 5.41 (s, 2H), 5.04-5.01 (m, 3H), 4.51 (t, J=9.0 Hz, 1H), 4.40-4.36 (m, 1H), 4.30-4.27 (m, 2H), 4.23-4.20 (m, 1H), 5.04-5.01 (m, 3H), 3.74-3.68 (m, 2H), 3.62-3.55 (m, 4H), 3.47-3.45 (m, 11H), 3.30-3.28 (m, 3H), 3.10-2.54 (m, 9H), 2.47-2.44 (m, 1H), 2.39-2.35 (m, 1H), 2.25-2.20 (m, 1H), 2.10-2.07 (m, 2H), 2.02-1.34 (m, 19H), 1.28-1.21 (m, 9H), 1.17 (t, J=7.0 Hz, 3H), 1.06 (s, 3H), 1.05 (s, 3H), 0.91 (d, J=6.5 Hz, 3H), 0.87-0.80 (m, 15H), 0.70 (s, 3H) ppm.

### LP14: (4S)-4-({2-[(1R,3R)-1-(Acetyloxy)-3-[(2S,3S)-2-{[(2R)-1,2-dimethylpyrrolidin-2-yl]formamido}-N-hexyl-3-methylpentanamido]-4-methylpentyl]-1,3-thiazol-4-yl}formamido)-5-(4-{[({4-[(2S)-2-[(2S)-2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methoxy)carbonyl]amino}phenyl)-2,2-dimethylpentanoic Acid (LP14)

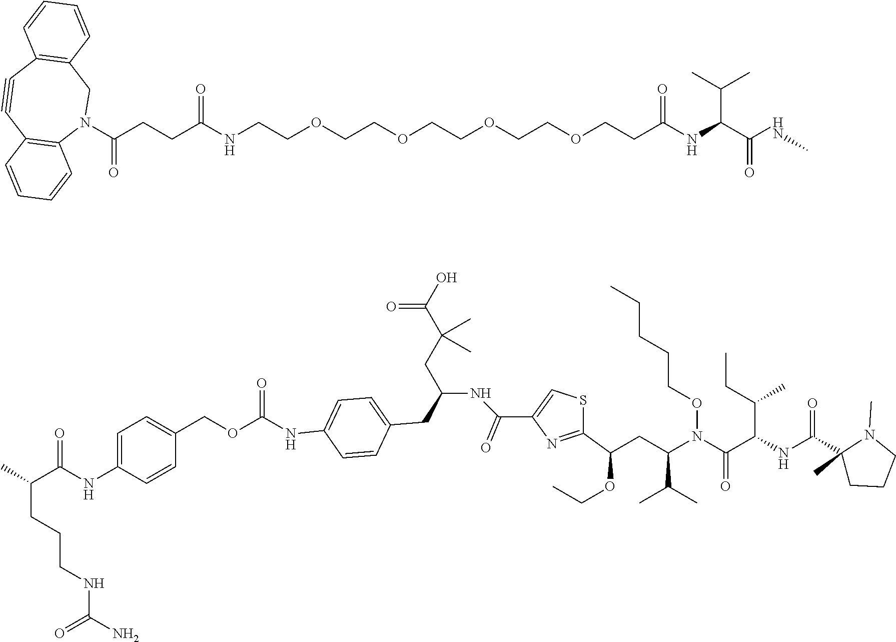

Following General Procedure IX from amine L1-3e and OSu ester L0-1c, linker-payload LP14 (6 mg, 54% yield) was obtained as a white solid. ESI m/z: 884 (M/2+H)⁺. ¹H NMR (500 MHz, DMSO_{d6}) δ 10.0 (s, 1H), 9.67 (s, 1H), 8.16 (s, 1H), 8.11 (d, J=7.0 Hz, 1H), 7.86 (d, J=8.5 Hz, 1H), 7.75-7.72 (m, 2H), 7.68-7.66 (m, 1H), 7.61 (d, J=8.0 Hz, 3H), 7.37-7.32 (m, 6H), 7.30-7.28 (m, 1H), 7.05 (d, J=8.5 Hz, 2H), 5.98-5.96 (m, 1H), 5.66-5.64 (m, 1H), 5.40 (s, 2H), 5.04 (s, 2H), 4.44 (t, J=9.5 Hz, 1H), 4.40-4.36 (m, 1H), 4.32-4.26 (m, 1H), 4.24-4.21 (m, 1H), 3.62-2.92 (m, 30H), 2.70-2.19 (m, 10H), 2.13 (s, 3H), 2.02-1.33 (m, 18H), 1.31-1.12 (m, 12H), 1.08 (s, 3H), 1.06 (s, 3H), 0.96 (d, J=6.5 Hz, 3H), 0.86-0.81 (m, 15H), 0.67 (d, J=5.0 Hz, 3H) ppm.

Synthesis of LP12 as in FIG. 12B.

### LP12: (4S)-4-({2-[(1R,3R)-1-(Acetyloxy)-3-[(2S,3S)—N-hexyl-3-methyl-2-{[(2R)-1-methylpiperidin-2-yl]formamido}pentanamido]-4-methylpentyl]-1,3-thiazol-4-yl}formamido)-5-[4-(2-{[({4-[(2S)-2-[(2S)-2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methoxy)carbonyl]amino}acetamido)phenyl]-2,2-dimethylpentanoic Acid (LP12)

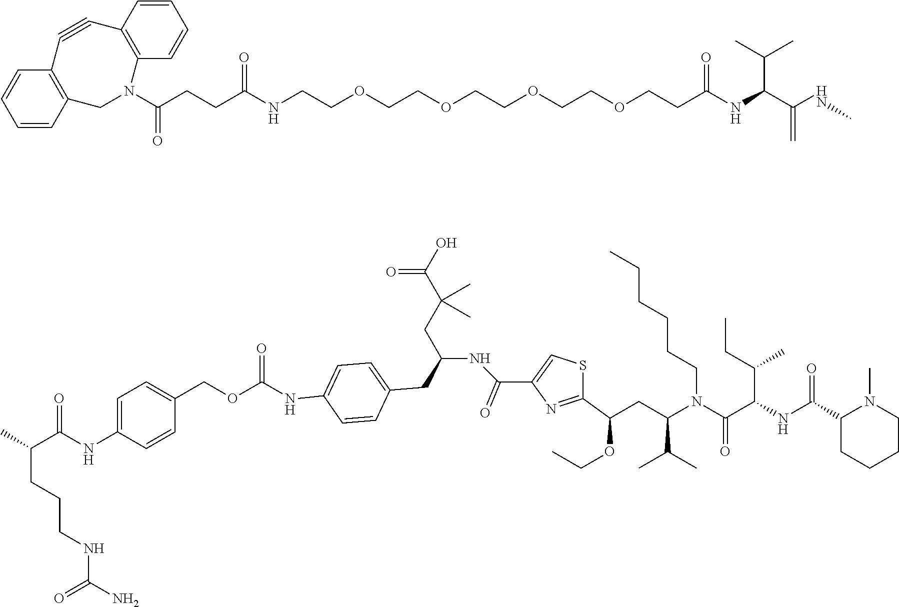

To a solution of payload P28 (70 mg, 79 μmol) in DMF (5 mL) was added compound L2-1 (86 mg, 79 μmol), HOBt (11 mg, 79 μmol) and DIPEA (31 mg, 0.24 mmol). The mixture was stirred at room temperature for an hour, and monitored by LCMS. The reaction mixture was purified directly by reversed phase flash chromatography (30-70% acetonitrile in aq. ammonium bicarbonate (10 mM)) to give linker-payload LP12 (27 mg, 19% yield) as a white solid. ESI: 913 (M/2+H)⁺. ¹H NMR (400 MHz, DMSO_{d6}) δ 12.13 (s, 1H), 9.99 (s, 1H), 9.87 (s, 1H), 8.15 (s, 1H), 8.12 (d, J=4.0 Hz, 1H), 7.87 (d, J=8.8 Hz, 1H), 7.75 (t, J=5.2 Hz, 1H), 7.69-7.57 (m, 6H), 7.52-7.44 (m, 5H), 7.40-7.27 (m, 5H), 7.10 (d, J=8.4 Hz, 2H), 5.97 (t, J=5.6 Hz, 1H), 5.65 (d, J=12.8 Hz, 1H), 5.41 (s, 2H), 5.05-5.01 (d, J=13.6 Hz, 1H), 4.97 (s, 2H), 4.49 (t, J=9.2 Hz, 1H), 4.41-4.35 (m, 1H), 4.27-4.21 (m, 2H), 3.76 (d, J=6.4 Hz, 2H), 3.64-3.56 (m, 3H), 3.48-3.45 (m, 13H), 3.29-3.28 (m, 2H), 3.11-3.06 (m, 2H), 3.05-2.93 (m, 4H), 2.84-2.67 (m, 3H), 2.59-2.54 (m, 1H), 2.46-2.44 (m, 1H), 2.40-2.32 (m, 2H), 2.25-2.20 (m, 2H), 2.13 (s, 3H), 2.07 (s, 3H), 2.03-1.86 (m, 7H), 1.80-1.70 (m, 4H), 1.62-1.60 (m, 4H), 1.54-1.51 (m, 1H), 1.46-1.36 (m, 4H), 1.29 (m, 7H), 1.06-1.05 (m, 7H), 0.96-0.94 (m, 3H), 0.87-0.80 (m, 17H), 0.69-0.68 (m, 3H) ppm.

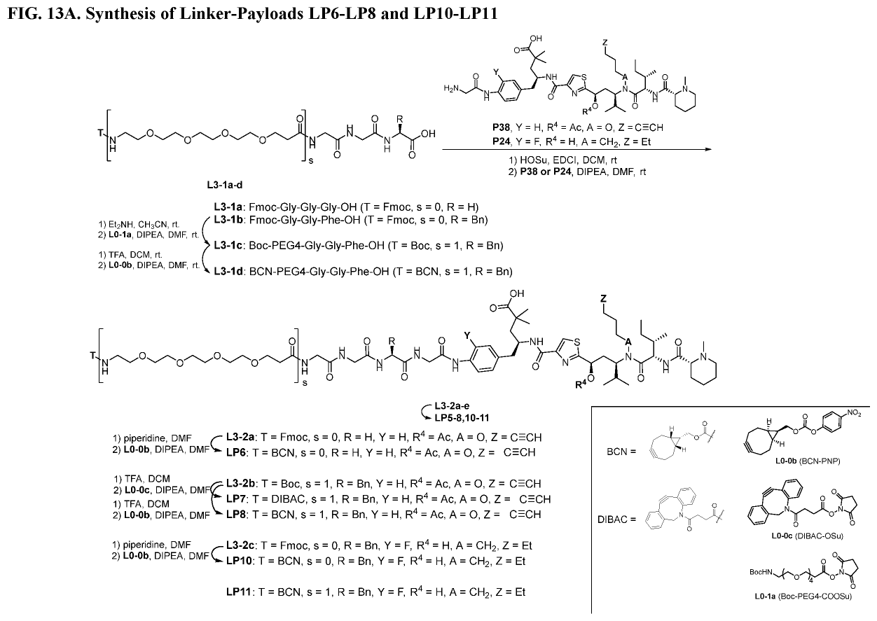

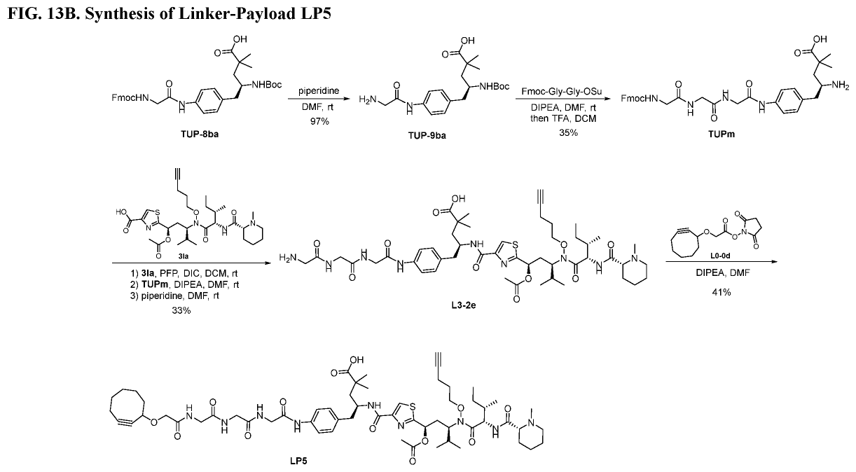

Synthesis of Peptide-linker-payloads LP6-LP8 and LP10-LP11 as in FIG. 13A.

### (4S)-4-({2-[(1R,3R)-1-(Acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-[4-(2-{2-[2-(2-{[(9H-fluoren-9-ylmethoxy)carbonyl]amino}acetamido)acetamido]acetamido}acetamido)phenyl]-2,2-dimethylpentanoic Acid (L3-2a)

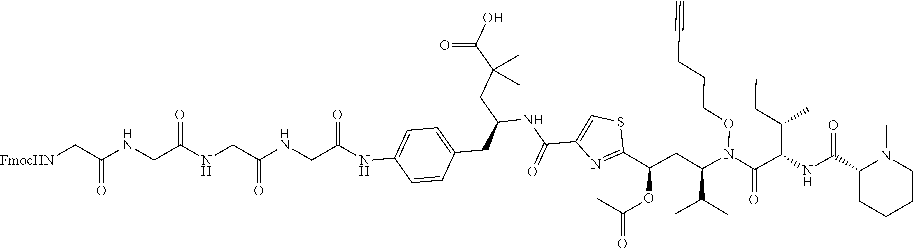

To a solution of Fmoc-Gly-Gly-Gly-OH (L3-1a) (0.40 g, 1.0 mmol) in DCM (40 mL) was added HOSu (0.25 g, 2.2 mmol) and EDCI (0.42 g, 2.2 mmol). The reaction mixture was stirred at room temperature for 24 hours. The resulting mixture was diluted with DCM (50 mL) and washed with water (50 mL). The organic phase was dried over anhydrous sodium sulfate and concentrated to give OSu ester (0.30 g, ESI m/z: 509 (M+H)⁺). OSu ester was used directly without further purification. Following General Procedure IX using the OSu ester (51 mg) and amine P38 (88 mg, 0.10 mmol), compound L3-2a (63 mg, 49% yield from P38) was obtained as a white solid. ESI m/z: 638 (M/2+H)⁺.

### LP6: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-{4-[2-(2-{2-[2-({[endo-bicyclo[6.1.0]non-4-yn-9-ylmethoxy]carbonyl}amino)acetamido]acetamido}acetamido)acetamido]phenyl}-2,2-dimethylpentanoic Acid (LP6)

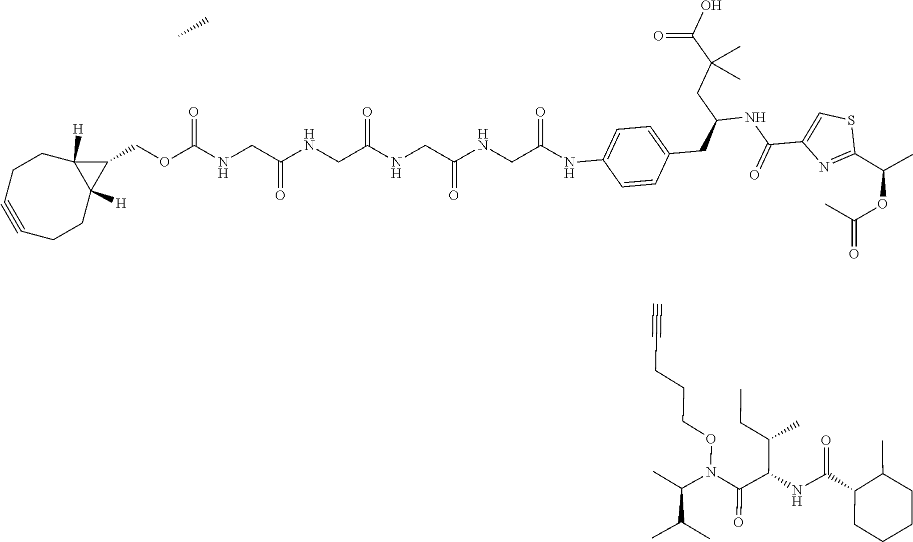

To a solution of L3-2a (25 mg, 20 μmol) in DMF (1 mL) was added piperidine (3.4 mg, 40 μmol), and the mixture was stirred at room temperature for 2 hours until Fmoc was totally removed according to LCMS. The resulting mixture was purified directly by reversed phase flash chromatography (10-95% acetonitrile in aq. ammonium bicarbonate (10 mM)) to give an amine (20 mg, ESI m/z: 527 (M/2+H)⁺) as a white solid. The amine was dissolved in DMF (1 mL). To the solution was added DIPEA (5.9 mg, 46 μmol) and compound L0-Ob (6.0 mg, 19 μmol), the mixture was stirred at room temperature for 2 hours, and monitored by LCMS. The resulting mixture was purified directly by reversed phase flash chromatography (0-70% acetonitrile in aq. ammonium bicarbonate (10 mM)) to give linker-payload LP6 (20 mg, 81% yield) as a white solid. ESI m/z: 615 (M/2+H)⁺. ¹H NMR (400 MHz, DMSO_{d6}) δ 9.80 (s, 1H), 8.25-8.20 (m, 2H), 8.15-8.10 (m, 2H), 7.85-7.80 (m, 1H), 7.65-7.60 (m, 1H), 7.50 (d, J=6.8 Hz, 2H), 7.45-7.40 (m, 1H), 7.10 (d, J=6.8 Hz, 2H), 5.85 (d, J=8.4 Hz, 1H), 4.75 (t, J=7.6 Hz, 1H), 4.30-4.25 (m, 2H), 4.10-4.00 (m, 3H), 3.90-3.85 (m, 2H), 3.75-3.70 (m, 3H), 3.65-3.60 (m, 2H), 2.80-2.60 (m, 5H), 2.30-2.10 (m, 5H), 2.10-2.00 (m, 11H), 2.00-1.65 (m, 8H), 1.70-1.10 (m, 13H), 1.07 (s, 3H), 1.03 (s, 3H), 0.98-0.95 (m, 3H), 0.90-0.80 (m, 10H) ppm.

### (2S)-2-{2-[2-(1-{[(tert-Butoxy)carbonyl]amino}-3,6,9,12-tetraoxapentadecan-15-amido)acetamido]acetamido}-3-phenylpropanoic Acid (L3-1c)

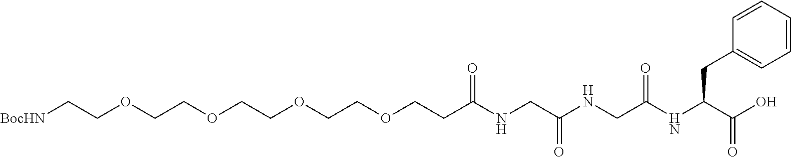 [SMILES: CC(C)(C)OC(=O)NCCOCCOCCOCCOCCC(=O)NCC(=O)NCC(=O)N[C@@H](Cc1ccccc1)C(=O)O]

To a solution of Fmoc-Gly-Gly-Phe-OH (L3-1b) (0.62 g, 1.2 mmol) in acetonitrile (5 mL) was added diethylamine (1 mL), the reaction mixture was stirred at room temperature for 3 hours, and monitored by LCMS. The volatiles were removed in vacuo and the residue (0.35 g, ESI m/z: 280 (M+H)⁺) was used for the amidation directly. Following General Procedure IX using the residue and OSu ester L0-la, Boc-PEG4-Gly-Gly-Phe-OH (L3-1c) (0.25 g, 32% yield) was obtained as a white solid after purification by reversed phase flash chromatography (0-100% acetonitrile in aq. TFA (0.05%)). ESI m/z: 627 (M+H)⁺.

### (4S)-4-({2-[(1R,3R)-1-(Acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-(4-{2-[(2S)-2-{2-[2-(1-{[(tert-butoxy)carbonyl]amino}-3,6,9,12-tetraoxapentadecan-15-amido)acetamido]acetamido}-3-phenylpropanamido]acetamido}phenyl)-2,2-dimethylpentanoic Acid (L3-2b)

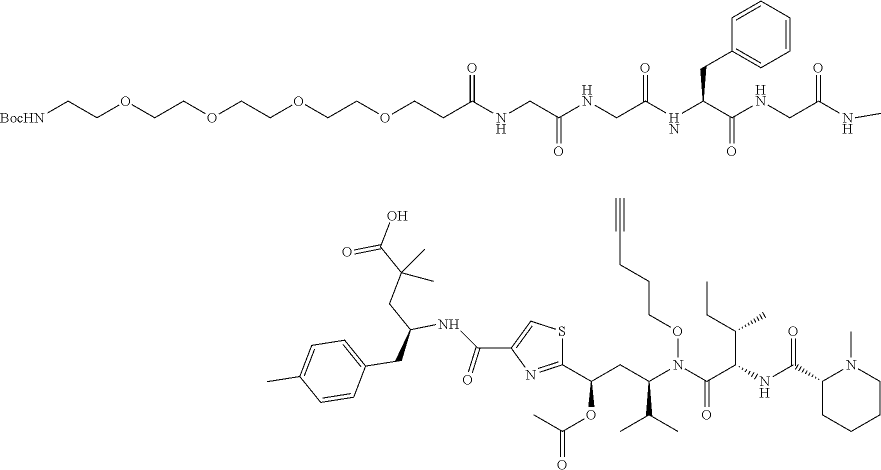

To a solution of Boc-PEG4-Gly-Gly-Phe-OH (L3-1c) (0.12 g, 0.20 mmol) in DCM (10 mL) were added HOSu (46 mg, 0.40 mmol) and EDCI (77 mg, 0.40 mmol), and the reaction mixture was stirred at room temperature for 2 hours. The resulting mixture was diluted with DCM (100 mL) and washed with water (50 mL). The organic phase was dried over anhydrous sodium sulfate and concentrated to give OSu ester (0.14 g, ESI m/z: 746 (M+Na)⁺). OSu ester was used directly without further purification. Following General Procedure IX using the OSu ester (98 mg) and amine P38 (80 mg, 91 μmol), compound L3-2b (0.10 g, 74% yield from P38) was obtained as a white solid. ESI m/z: 696 ((M−Boc)/2+H)⁺.

### LP7: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-(4-{2-[(2S)-2-(2-{2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]acetamido}acetamido)-3-phenylpropanamido]acetamido}phenyl)-2,2-dimethylpentanoic Acid (LP7)

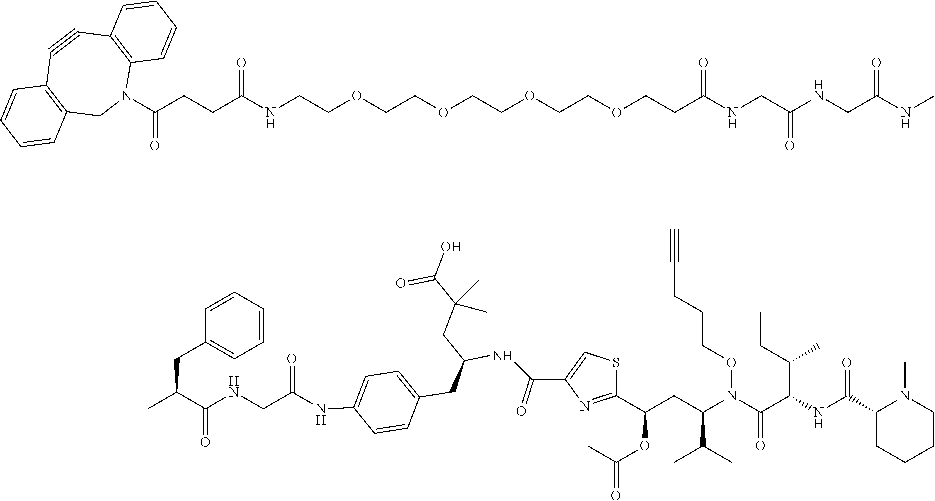

To a solution of L3-2b (1.0 g, 0.67 mmol) in DCM (20 mL) was added TFA (5 mL), and the mixture was stirred at room temperature for 2 hours until Boc was totally removed according to LCMS. The resulting mixture was purified directly by reversed phase flash chromatography (0-100% acetonitrile in aq. TFA (0.05%)) to give amine L3-3b (0.30 g, ESI m/z: 696 (M/2+H)⁺) as a white solid. Following General Procedure IX using amine L3-3b (80 mg) and compound L0-0c (24 mg, 60 μmol), linker-payload LP7 (25 mg, 8% yield from L3-2b) was obtained as a white solid after purification by reversed phase flash chromatography (0-100% acetonitrile in aq. ammonium bicarbonate (0.05%)). ESI m/z: 839 (M/2+H)⁺. ¹H NMR (400 MHz, DMSO_{d6}) δ 9.77 (s, 1H), 8.41-8.40 (m, 1H), 8.21-8.17 (m, 3H), 8.06-8.05 (m, 1H), 7.79-7.77 (m, 1H), 7.69-7.67 (m, 1H), 7.63-7.56 (m, 2H), 7.51-7.43 (m, 5H), 7.40-7.28 (m, 3H), 7.25-7.24 (m, 4H), 7.19-7.16 (m, 1H), 7.13-7.11 (m, 2H), 5.90-5.75 (m, 1H), 5.05-5.00 (m, 1H), 4.78-4.73 (m, 1H), 4.52-4.49 (m, 1H), 4.26-4.24 (m, 1H), 4.17-4.03 (m, 3H), 3.87-3.84 (m, 2H), 3.79-3.74 (m, 1H), 3.69-3.68 (m, 2H), 3.62-3.57 (m, 4H), 3.46-3.42 (m, 13H), 3.29-3.27 (m, 2H), 3.10-3.03 (m, 3H), 2.86-2.79 (m, 4H), 2.68-2.54 (m, 2H), 2.40-2.32 (m, 6H), 2.27-2.20 (m, 1H), 2.12 (s, 3H), 2.09 (s, 3H), 2.03-1.92 (m, 4H), 1.84-1.72 (m, 4H), 1.63-1.05 (m, 10H), 1.02-0.95 (m, 9H), 0.89-0.80 (m, 9H) ppm.

### LP8: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-(4-{2-[(2S)-2-(2-{2-j[1-({[endo-bicyclo[6.1.0]non-4-yn-9-ylmethoxy]carbonyl}amino)-3,6,9,12-tetraoxapentadecan-15-amido]acetamido}acetamido)-3-phenylpropanamido]acetamido}phenyl)-2,2-dimethylpentanoic Acid (LP8)

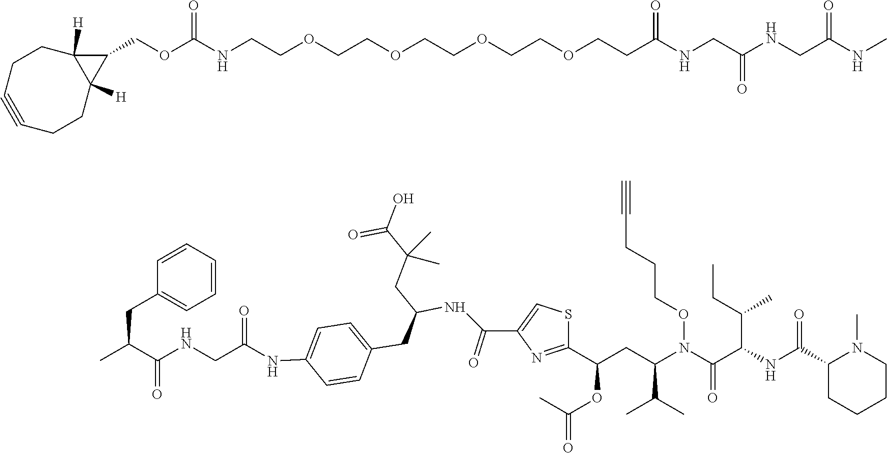

To a solution of amine L3-3b (27 mg, 19 μmol; obtained above) in DMF (3 mL) was added HOBt (1.4 mg, 10 μmol), DIPEA (8.0 mg, 62 μmol) and compound L0-Ob (13 mg, 41 μmol). The mixture was stirred at room temperature for 2 hours, and monitored by LCMS. The resulting mixture was purified directly by reversed phase flash chromatography (0-100% acetonitrile in aq. ammonium bicarbonate (0.05%)) to give linker-payload LP8 (24 mg, 25% yield from L3-2b) as a white solid. ESI m/z: 784 (M/2+H)⁺. ¹H NMR (400 MHz, DMSO_{d6}) δ 9.76 (d, J=4.8 Hz, 1H), 8.41-8.38 (m, 1H), 8.20-8.16 (m, 2H), 8.05-8.04 (m, 1H), 7.87-7.84 (m, 1H), 7.57 (d, J=10.0 Hz, 1H), 7.48 (d, J=8.8 Hz, 2H), 7.26-7.24 (m, 4H), 7.21-7.15 (m, 1H), 7.13-7.10 (m, 3H), 5.81 (d, J=10.4 Hz, 1H), 4.78-4.74 (m, 1H), 4.53-4.47 (m, 1H), 4.28-4.20 (m, 2H), 4.07-4.02 (m, 4H), 3.89-3.78 (m, 3H), 3.70-3.68 (m, 2H), 3.63-3.56 (m, 3H), 3.48-3.47 (m, 14H), 3.21-3.03 (m, 4H), 2.85-2.78 (m, 4H), 2.43-2.30 (m, 8H), 2.26-2.10 (m, 11H), 2.05-1.91 (m, 6H), 1.84-1.76 (m, 4H), 1.66-1.60 (m, 3H), 1.55-1.33 (m, 8H), 1.06-1.03 (m, 6H), 0.96-0.95 (m, 3H), 0.89-0.81 (m, 9H) ppm.

### (4S)-5-(4-{2-[(2S)-2-[2-(2-{[(9H-Fluoren-9-ylmethoxy)carbonyl]amino}acetamido)acetamido]-3-phenylpropanamido]acetamido}-3-fluorophenyl)-4-({2-[(1R,3R)-3-[(2S,3S)—N-hexyl-3-methyl-2-{[(2R)-1-methylpiperidin-2-yl]formamido}pentanamido]-1-hydroxy-4-methylpentyl]-1,3-thiazol-4-yl}formamido)-2,2-dimethylpentanoic Acid (L3-2c)

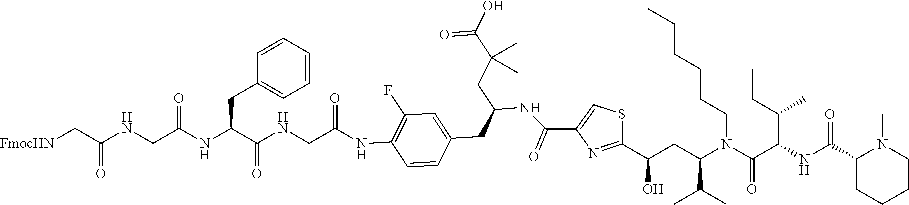

To a solution of Fmoc-Gly-Gly-Phe-OH (L3-1b) (0.10 g, 0.20 mmol) in DCM (10 mL) was added HOSu (46 mg, 0.40 mmol) and EDCI (77 mg, 0.40 mmol). The reaction mixture was stirred at room temperature for 4 hours. The resulting mixture was diluted with DCM (50 mL) and washed with water (50 mL). The organic phase was dried over anhydrous sodium sulfate and concentrated in vacuo. The residue was purified by reversed phase flash chromatography (0-50% acetonitrile in water) to give OSu ester (54 mg, ESI m/z: 599 (M+H)⁺) as a white solid. Following General Procedure IX using the OSu ester (54 mg) and amine P24 (75 mg, 87 μmol), compound L3-2c (25 mg, 21% yield from P24) was obtained as a white solid. ESI m/z: 672 (M/2+H)⁺.

### LP10: (4S)-5-(4-{2-[(2S)-2-{2-[2-({[endo-bicyclo[6.1.0]non-4-yn-9-ylmethoxy]carbonyl}amino)acetamido]acetamido}-3-phenylpropanamido]acetamido}-3-fluorophenyl)-4-({2-[(1R,3R)-3-[(2S,3S)—N-hexyl-3-methyl-2-{[(2R)-1-methylpiperidin-2-yl]formamido}pentanamido]-1-hydroxy-4-methylpentyl]-1,3-thiazol-4-yl}formamido)-2,2-dimethylpentanoic Acid (LP10)

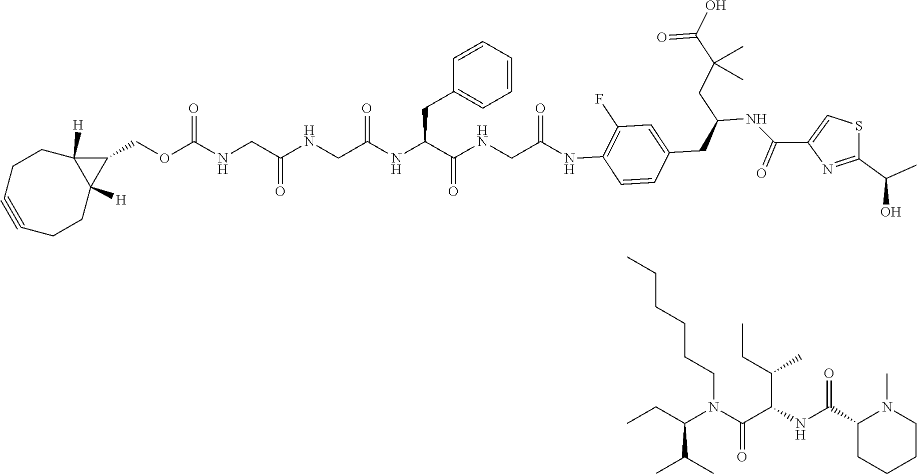

To a solution of L3-2c (25 mg, 19 μmol) in DMF (1 mL) was added piperidine (6.0 mg, 74 μmol), and the mixture was stirred at room temperature for 3 hours until Fmoc was totally removed according to LCMS. The resulting mixture was purified directly by reversed phase flash chromatography (10-95% acetonitrile in aq. ammonium bicarbonate (10 mM)) to give an amine (17 mg, ESI m/z: 561 (M/2+H)⁺) as a white solid. The amine was dissolved in DMF (3 mL). To the solution was added HOBt (3.0 mg, 22 μmol), DIPEA (8.0 mg, 62 μmol), and compound L0-Ob (10 mg, 30 μmol). The mixture was stirred at room temperature for 3 hours, and monitored by LCMS. The resulting mixture was directly purified by reversed phase flash chromatography (10-95% acetonitrile in aq. ammonium bicarbonate (10 mM)) to give linker-payload LP10 (7.8 mg, 40% yield) as a white solid. ESI m/z: 649 (M/2+H)⁺. ¹H NMR (400 MHz, DMSO_{d6}) δ 9.64 (s, 1H), 8.42 (t, J=5.6 Hz, 1H), 8.17 (d, J=9.2 Hz, 1H), 8.09 (s, 1H), 7.99 (t, J=6.0 Hz, 1H), 7.78 (t, J=8.0 Hz, 2H), 7.36 (t, J=6.0 Hz, 1H), 7.26-7.22 (m, 5H), 7.19-7.15 (m, 1H), 7.06 (d, J=12.0 Hz, 1H), 6.97 (d, J=8.0 Hz, 1H), 4.56-4.51 (m, 3H), 4.26-4.19 (m, 1H), 4.05 (d, J=8.0 Hz, 2H), 3.95-3.91 (m, 2H), 3.78 (d, J=5.6 Hz, 1H), 4.05 (d, J=6.0 Hz, 1H), 3.61-3.58 (m, 3H), 3.10-3.08 (m, 1H), 3.06-3.04 (m, 1H), 2.85-2.75 (m, 5H), 2.23-2.21 (m, 1H), 2.18-2.16 (m, 1H), 2.15-2.12 (m, 3H), 2.11-2.09 (m, 1H), 2.06 (s, 3H), 1.95-1.90 (m, 2H), 1.87-1.79 (m, 3H), 1.57-1.47 (m, 6H), 1.33-1.29 (m, 6H), 1.26-1.23 (m, 3H), 1.16-1.11 (m, 2H), 1.07-1.01 (m, 7H), 0.92-0.79 (m, 19H), 0.75-0.70 (m, 3H) ppm. ¹⁹F NMR (376 MHz, DMSO_{d6}) 6-132.9 ppm.

### (2S)-2-(2-{2-[1-({[endo-Bicyclo[6.1.0]non-4-yn-9-ylmethoxy]carbonyl}amino)-3,6,9,12-tetraoxapentadecan-15-amido]acetamido}acetamido)-3-phenylpropanoic Acid (L3-1d)

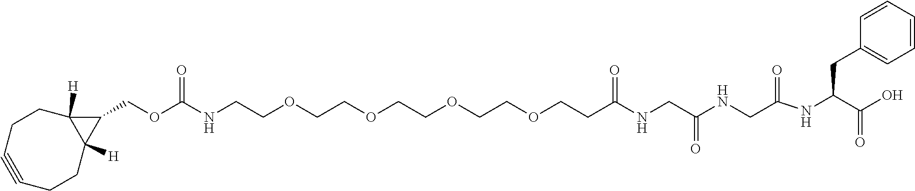 [SMILES: O=C(CCOCCOCCOCCOCCNC(=O)OC[C@@H]1[C@@H]2CCC#CCC[C@@H]21)NCC(=O)NCC(=O)N[C@@H](Cc1ccccc1)C(=O)O]

To a solution of Boc-PEG4-Gly-Gly-Phe-OH (L3-1c) (50 mg, 80 μmol) in DCM (3 mL) was added TFA (1 mL), and the mixture was stirred at room temperature for 3 hours until Boc was totally removed according to LCMS. The resulting mixture was concentrated in vacuo and lyophilized to give a residue (ESI m/z: 527 (M+H)⁺). The residue was dissolved in DMF (3 mL). To the solution was added HOBt (12 mg, 85 μmol), DIPEA (22 mg, 0.17 mmol), and compound L0-Ob (27 mg, 85 μmol). The mixture was stirred at room temperature for 3 hours, and monitored by LCMS. The resulting mixture was directly purified by reversed phase flash chromatography (0-70% acetonitrile in water) to give BCN-PEG4-Gly-Gly-Phe-OH (25 mg, 44% yield) as a white solid. ESI m/z: 703 (M+H)⁺.

### LP11: (4S)-5-(4-{2-[(2S)-2-(2-{2-[1-({[endo-bicyclo[6.1.0]non-4-yn-9-ylmethoxy]carbonyl}amino)-3,6,9,12-tetraoxapentadecan-15-amido]acetamido}acetamido)-3-phenylpropanamido]acetamido}-3-fluorophenyl)-4-({2-[(1R,3R)-3-[(2S,3S)—N-hexyl-3-methyl-2-{[(2R)-1-methylpiperidin-2-yl]formamido}pentanamido]-1-hydroxy-4-methylpentyl]-1,3-thiazol-4-yl}formamido)-2,2-dimethylpentanoic Acid (LP11)

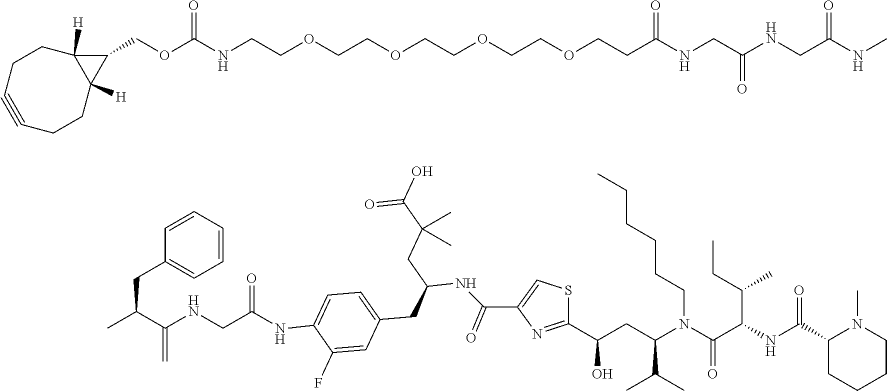

To a solution of BCN-PEG4-Gly-Gly-Phe-OH (L3-1d) (25 mg, 36 μmol) in DCM (3 mL) was added HOSu (8.0 mg, 72 μmol) and EDCI (14 mg, 72 μmol). The reaction mixture was stirred at room temperature for 3 hours. The resulting mixture was concentrated in vacuo and the residue was purified by reversed phase flash chromatography (0-50% acetonitrile in water) to give OSu ester (13 mg, ESI m/z: 822 (M+Na)⁺) as a white solid. Following General Procedure IX using the OSu ester (13 mg) and amine P24 (14 mg, 16 μmol), linker-payload LP11 (3.8 mg, 15% yield from P24) was obtained as a white solid. ESI m/z: 773 (M/2+H)⁺. ¹H NMR (400 MHz, DMSO_{d6}) δ 9.62 (s, 1H), 8.38 (s, 1H), 8.18 (t, J=4.4 Hz, 1H), 8.12 (d, J=8.4 Hz, 1H), 8.07 (s, 1H), 8.04-7.97 (m, 1H), 7.78 (t, J=8.4 Hz, 1H), 7.26-7.22 (m, 4H), 7.19-7.15 (m, 1H), 7.12-7.08 (m, 1H), 7.05 (d, J=12.8 Hz, 1H), 6.96 (d, J=8.8 Hz, 1H), 6.73-6.63 (m, 1H), 6.31-6.26 (m, 1H), 5.76 (s, 1H), 4.56-4.51 (m, 2H), 4.02 (d, J=8.0 Hz, 2H), 3.95-3.91 (m, 2H), 3.76 (d, J=6.0 Hz, 1H), 3.73 (d, J=6.0 Hz, 1H), 3.69 (d, J=5.6 Hz, 2H), 3.62-3.57 (m, 3H), 3.50-3.46 (m, 13H), 3.18-3.16 (m, 1H), 3.14-3.08 (m, 4H), 3.06-3.03 (m, 1H), 2.84-2.75 (m, 4H), 2.39 (t, J=6.8 Hz, 3H), 2.35-2.31 (m, 1H), 2.24-2.21 (m, 1H), 2.20-2.18 (m, 1H), 2.17-2.12 (m, 4H), 2.10-2.07 (m, 1H), 2.06 (s, 2H), 2.01-1.99 (m, 1H), 1.86-1.81 (m, 2H), 1.55-1.47 (m, 5H), 1.40-1.38 (m, 1H), 1.37-1.34 (m, 1H), 1.33-1.28 (m, 6H), 1.27-1.22 (m, 5H), 1.08-1.02 (m, 7H), 0.93-0.78 (m, 19H), 0.76-0.67 (m, 3H) ppm. ¹⁹F NMR (376 MHz, DMSO_{d6}) δ−135.4 ppm.

Synthesis of Peptide-linker-payload LP5 as in FIG. 13B.

### (4S)-5-[4-(2-Aminoacetamido)phenyl]-4-{[(tert-butoxy)carbonyl]amino}-2,2-dimethylpentanoic Acid (TUP-9ba)

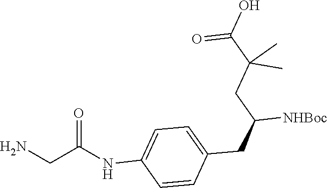 [SMILES: CC(C)(C)OC(=O)N[C@@H](Cc1ccc(NC(=O)CN)cc1)CC(C)(C)C(=O)O]

To a solution of TUP-8ba (0.80 g, 1.3 mmol) in DMF (3 mL) was added piperidine (0.33 g, 3.9 mmol), and the reaction mixture was stirred at room temperature for 2 hours until Fmoc was totally removed according to LCMS. The resulting mixture was purified directly by reversed phase flash chromatography (0-30% acetonitrile in aq. ammonium bicarbonate (10 mM)) to give compound TUP-9ba (0.50 g, 97% yield) as a white solid. ESI m/z: 787 (2M+H)⁺. ¹H NMR (400 MHz, DMSO_{d6}) δ 9.85 (s, 1H), 7.51 (d, J=8.0 Hz, 2H), 7.06 (d, J=8.4 Hz, 2H), 6.60 (d, J=8.4 Hz, 1H), 3.67-3.61 (m, 1H), 3.25 (s, 2H), 2.85-2.81 (m, 1H), 1.74-1.65 (m, 1H), 1.55-1.48 (m, 2H), 1.30 (s, 9H), 1.21 (s, 2H), 1.01 (s, 6H) ppm.

### (4S)-4-Amino-5-(4-{2-[2-(2-{[(9H-fluoren-9-ylmethoxy)carbonyl]amino}acetamido)acetamido]acetamido}phenyl)-2,2-dimethylpentanoic Acid (TUPm)

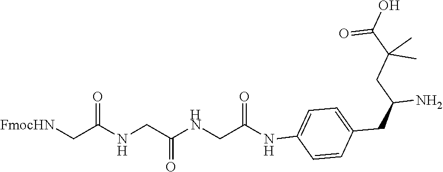

To a solution of Fmoc-Gly-Gly-OH (L3-le) (0.25 g, 0.64 mmol) in DCM (5 mL) were added HOSu (0.16 g, 1.4 mmol) and EDCI (0.27 g, 1.4 mmol). The reaction mixture was stirred at room temperature for 4 hours. The resulting mixture was concentrated in vacuo and the residue was purified by reversed phase flash chromatography (0-40% acetonitrile in water) to give OSu ester (0.32 g, ESI m/z: 452 (M+H)⁺) as a white solid. Following General Procedure IX using the OSu ester (0.32 g) and amine TUP-9ba (0.25 g, 0.64 mmol), compound TUPm (0.16 g, 35% yield from TUP-9ba) was obtained as a white solid. ESI m/z: 630 (M+H)⁺.

### (4S)-4-({2-[(1R,3R)-1-(Acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-(4-{2-[2-(2-aminoacetamido)acetamido]acetamido}phenyl)-2,2-dimethylpentanoic Acid (L3-2e)

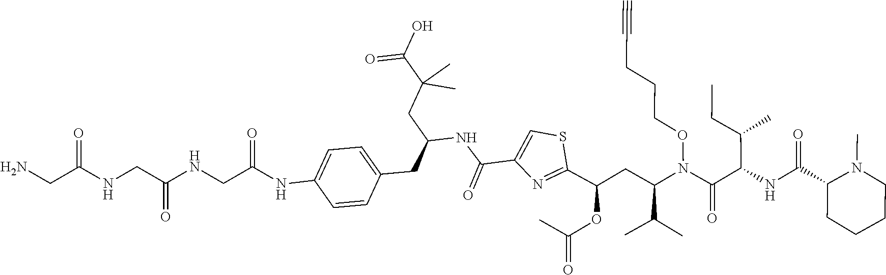

Following General Procedure VI from 3Ia (90 mg, 0.15 mmol) with TUPm, compound Fmoc-L3-2e (90 mg, ESI m/z: 610 (M/2+H)⁺) was obtained as a white solid. Fmoc-L3-2e was dissolved in DMF (3 mL). To the solution was added piperidine (25 mg, 0.30 mmol), and the reaction mixture was stirred at room temperature for 3 hours until Fmoc was totally removed according to LCMS. The resulting mixture was purified directly by reversed phase flash chromatography (0-50% acetonitrile in water) to give compound L3-2e (50 mg, 33% yield from 3Ia) as a white solid. ESI m/z: 997 (M+H)⁺.

### LP5: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-{4-[2-(2-{2-[2-(cyclooct-2-yn-1-yloxy)acetamido]acetamido}acetamido)acetamido]phenyl}-2,2-dimethylpentanoic Acid (LP5)

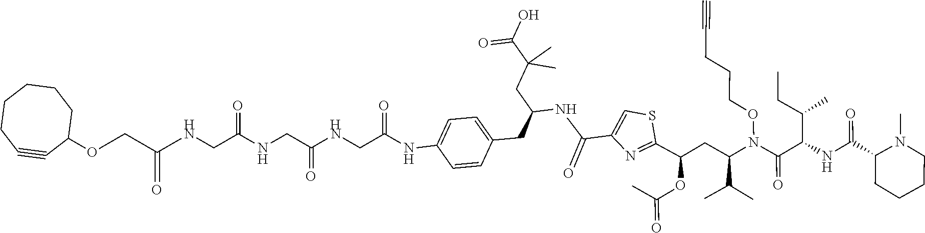

Following General Procedure IX using amine L3-2e (50 mg, 50 μmol) with OSu ester L0-Od (28 mg, 0.10 mmol), linker-payload LP5 (23 mg, 41% yield) was obtained as a white solid after purification by prep-HPLC (0-100% acetonitrile in aq. ammonium bicarbonate (10 mM)). ESI m/z: 1161 (M+H)⁺. ¹H NMR (400 MHz, DMSO_{d6}) δ 9.70 (s, 1H), 8.30 (t, J=4.8 Hz, 1H), 8.24 (t, J=5.2 Hz, 1H), 8.16 (s, 1H), 7.86 (t, J=4.8 Hz, 1H), 7.76 (d, J=9.6 Hz, 1H), 7.60 (s, 1H), 7.48 (d, J=8.0 Hz, 2H), 7.11 (d, J=7.6 Hz, 2H), 5.82 (d, J=10.0 Hz, 1H), 4.76 (t, J=8.4 Hz, 1H), 4.34-4.30 (m, 1H), 4.28-4.22 (m, 2H), 4.10-4.04 (m, 2H), 3.96-3.91 (m, 1H), 3.87-3.84 (m, 2H), 3.83-3.74 (m, 6H), 2.87-2.83 (m, 2H), 2.81-2.76 (m, 1H), 2.71-2.66 (m, 1H), 2.39-2.32 (m, 3H), 2.2-2.19 (m, 1H), 2.18-2.15 (s, 1H), 2.13 (s, 3H), 2.11 (s, 3H), 2.00-1.90 (m, 4H), 1.87-1.55 (m, 13H), 1.45-1.35 (m, 4H), 1.08-1.03 (m, 7H), 0.96 (d, J=6.0 Hz, 3H), 0.90-0.81 (m, 11H) ppm.

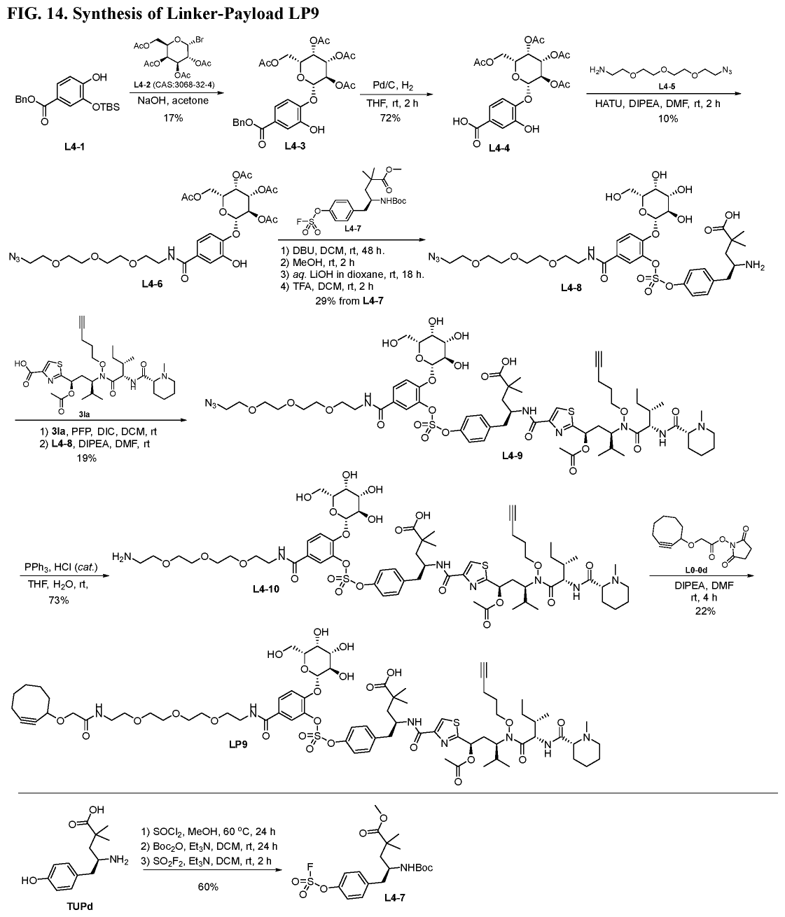

Synthesis of HOPAS-linker-payload LP9 as in FIG. 14.

### Benzyl 3-hydroxy-4-{[(2S,3R,4S,5S,6R)-3,4,5-tris(acetyloxy)-6-[(acetyloxy)methyl]oxan-2-yl]oxy}benzoate (L4-3)

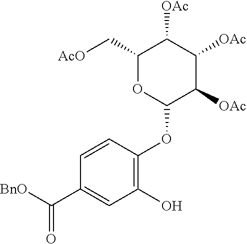

To a solution of compound L4-1 (0.36 g, 1.0 mmol) in acetone (5 mL) was added compound L4-2 (CAS: 3068-32-4, 0.53 g, 1.3 mmol) and aq. sodium hydroxide (1.1 M, 1 mL). The reaction mixture was stirred at room temperature for 24 hours, and monitored by LCMS. The volatiles were removed in vacuo and the residual aq. solution was purified by reversed phase flash chromatography (0-100% acetonitrile in aq. TFA (0.01%)) to give compound L4-3 (0.10 g, 17% yield) as a colorless oil. ESI m/z: 592 (M+18)*.

### 3-Hydroxy-4-{[(2S,3R,4S,5S,6R)-3,4,5-tris(acetyloxy)-6-[(acetyloxy)methyl]oxan-2-yl]oxy}benzoic Acid (L4-4)

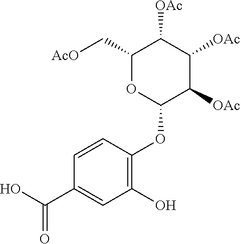 [SMILES: CC(=O)OC[C@H]1O[C@@H](Oc2ccc(C(=O)O)cc2O)[C@H](OC(C)=O)[C@@H](OC(C)=O)[C@H]1OC(C)=O]

To a solution of compound L4-3 (57 mg, 99 μmol) in THE (5 mL) was added palladium on carbon (containing 10% palladium, 6 mg, 10 wt %) under nitrogen. The reaction mixture was purged with hydrogen 3 times, stirred at room temperature under a hydrogen balloon for 2 hours, and monitored by LCMS. The resulting mixture was filtered through Celite and the filtrate was concentrated in vacuo. The residual oil was purified by reversed phase flash chromatography (0-100% acetonitrile in aq. TFA (0.01%)) to give compound L4-4 (35 mg, 72% yield) as a white solid. ESI m/z: 485 (M+H)⁺.

### [(2R,3S,4S,5R,6S)-3,4,5-Tris(acetyloxy)-6-{4-[(2-{2-[2-(2-azidoethoxy)ethoxy]ethoxy}ethyl)carbamoyl]-2-hydroxyphenoxy}oxan-2-yl]methyl Acetate (L4-6)

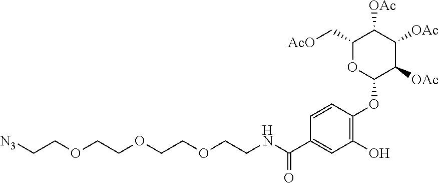

To a solution of compound L4-4 (35 mg, 72 μmol) in DMF (1 mL) was added HATU (16 mg, 72 μmol) and DIPEA (18 mg, 0.14 mmol). The reaction mixture was stirred at room temperature for 10 minutes before the addition of amine L4-5 (16 mg, 72 μmol). The mixture was stirred at room temperature for 2 hours, and monitored by LCMS. The resulting mixture was purified directly by reversed phase flash chromatography (0-100% acetonitrile in aq. TFA (0.01%)) to give compound L4-6 (5.0 mg, 10% yield) as a white solid. ESI m/z: 685 (M+H)⁺.

### (Methyl (4S)-4-{[(tert-butoxy)carbonyl]amino}-5-{4-[(fluorosulfonyl)oxy]phenyl}-2,2-dimethylpentanoate (L4-7)

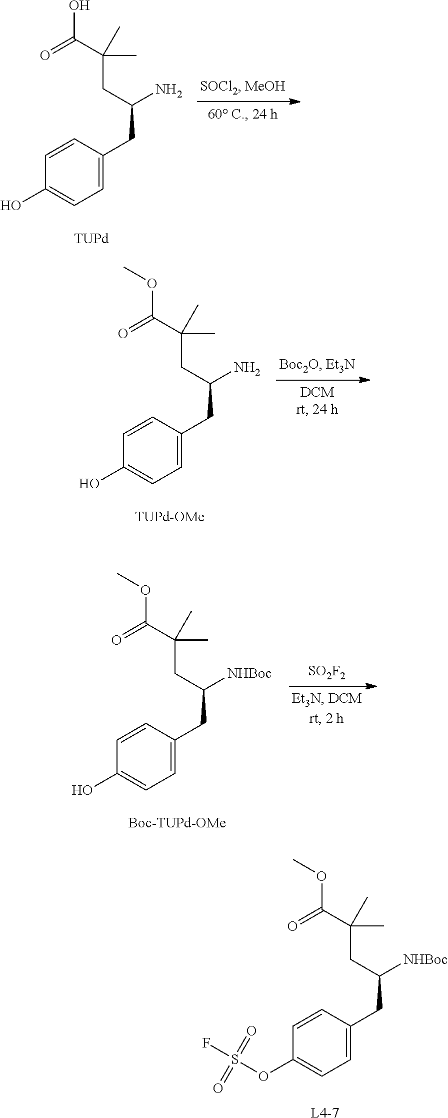

To a solution of TUPd (0.24 mg, 1.0 mmol) in methanol (3 mL) was added thionyl chloride (24 mg). The reaction mixture was stirred at 60° C. for 24 hours, and monitored by LCMS. The volatiles were removed in vacuo and the residual oil (0.26 g, ESI m/z: 296 (M+H)⁺) was dissolved in DCM (2 mL). To the solution was added triethylamine (0.22 g, 2.2 mmol) and Boc₂O (0.44 g, 2.0 mmol). The mixture was stirred at room temperature for 24 hours, and monitored by LCMS. The volatiles were removed in vacuo and the residue was purified by reversed phase flash chromatography (0-100% acetonitrile in aq. ammonium bicarbonate (10 mM)) to give Boc-TUPd-OMe (0.26 g, ESI m/z: 352 (M+H)⁺) as colorless oil. Boc-TUPd-OMe was dissolved in DCM (20 mL). To the solution was added triethylamine (76 mg, 0.75 mmol), and sulfuryl fluoride (0.5-1.0 L) was bubbled through the stirred solution at room temperature for 2 hours. The reaction was monitored by LCMS. The volatiles were removed in vacuo to give crude L4-7 (0.26 g, 60% yield from TUPd), and was used in the next step without further purification. ESI m/z: 434 (M+H)⁺.

### (4S)-4-Amino-5-{4-[({5-[(2-{2-[2-(2-azidoethoxy)ethoxy]ethoxy}ethyl)carbamoyl]-2-{[(2S,3R,4S,5R,6R)-3,4,5-trihydroxy-6-(hydroxymethyl)oxan-2-yl]oxy}phenoxy}sulfonyl)oxy]phenyl}-2,2-dimethylpentanoic Acid (L4-8)

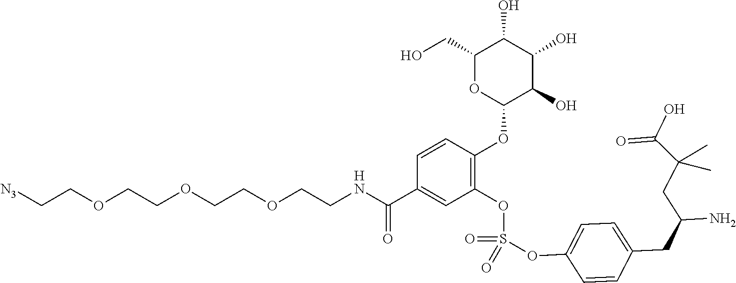

To a solution of compound L4-6 (68 mg, 0.10 mmol) in DCM (2 mL) was added DBU (76 mg, 0.2 mmol) and compound L4-7 (43 mg, 0.10 mmol). The reaction mixture was stirred at room temperature for 48 hours, and monitored by LCMS. To the reaction solution was then added methanol (2 mL), and the mixture was stirred at room temperature for 2 hours. The volatiles were removed in vacuo and the residue was purified by reversed phase flash chromatography (0-100% acetonitrile in aq. TFA (0.01%)) to give a colorless oil (40 mg, ESI m/z: 830 (M−Boc+H)⁺). The colorless oil was dissolved in ethanol (2 mL). To the solution was added aq. lithium hydroxide (2 mL, 66 mM), and the reaction mixture was stirred at room temperature for 18 hours. To the resulting mixture was added diluted aq. hydrochloride (1 M) to adjust the pH to pH 7.0. The volatiles were removed in vacuo and the residue was purified by reversed phase flash chromatography (0-100% acetonitrile in aq. TFA (0.01%)) to give Boc-L4-8 (30 mg, ESI m/z: 816 (M−Boc+H)⁺). Boc-L4-8 was dissolved in DCM (2 mL). To the solution was added TFA (0.2 mL), and the mixture was stirred at room temperature for 2 hours until Boc was totally removed according to LCMS. The resulting mixture was concentrated in vacuo and the residual oil was purified by reversed phase flash chromatography (0-100% acetonitrile in aq. TFA (0.01%)) to give compound L4-8 (24 mg, 29% yield from L4-6) as a white solid. ESI m/z: 816 (M+H)⁺.

### (4S)-4-({2-[(1R,3R)-1-(Acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-{4-[({5-[(2-{2-[2-(2-azidoethoxy)ethoxy]ethoxy}ethyl)carbamoyl]-2-{[(2S,3R,4S,5R,6R)-3,4,5-trihydroxy-6-(hydroxymethyl)oxan-2-yl]oxy}phenoxy}sulfonyl)oxy]phenyl}-2,2-dimethylpentanoic Acid (L4-9)

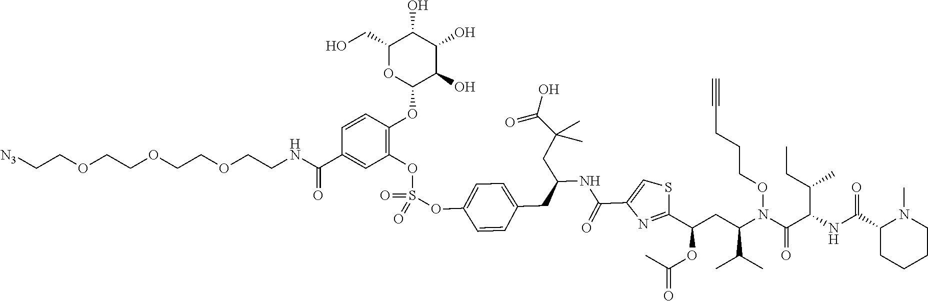

Following General Procedure VI from acid 3Ia (15 mg, 25 μmol) with amine L4-8, compound L4-9 (6.6 mg, 19% yield from 3Ia) was obtained as a white solid. ESI m/z: 703 (M/2+H)⁺.

### (4S)-4-({2-[(1R,3R)-1-(Acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-{4-[({5-[(2-{2-[2-(2-aminoethoxy)ethoxy]ethoxy}ethyl)carbamoyl]-2-{[(2S,3R,4S,5R,6R)-3,4,5-trihydroxy-6-(hydroxymethyl)oxan-2-yl]oxy}phenoxy}sulfonyl)oxy]phenyl}-2,2-dimethylpentanoic Acid (L4-10)

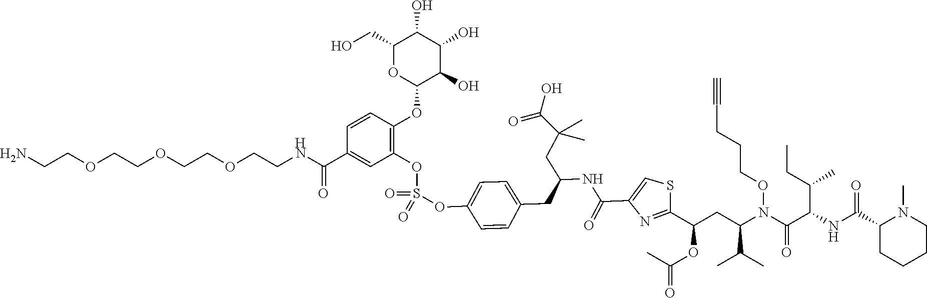

To a solution of compound L4-9 (4.2 mg, 3.0 μmol) in DMF (1.0 mL) was added triphenylphosphine (1.5 mg, 5.8 μmol) and a drop of water (˜0.02 mL). The reaction mixture was stirred at room temperature for 2 hours, and monitored by LCMS. The reaction mixture was directly purified by reversed phase flash chromatography (0-100% acetonitrile in aq. TFA (0.01%)) to give compound L4-10 (3.0 mg, 73% yield) as a white solid. ESI m/z: 691 (M/2+H)⁺.

### LP9: (4S)-4-({2-[(1R,3R)-1-(acetyloxy)-4-methyl-3-[(2S,3S)-3-methyl-2-{[(2R)-1-methylpiperidin-2-yl]formamido}-N-(pent-4-yn-1-yloxy)pentanamido]pentyl]-1,3-thiazol-4-yl}formamido)-5-[4-({[5-({2-[2-(2-{2-[2-(cyclooct-2-yn-1-yloxy)acetamido]ethoxy}ethoxy)ethoxy]ethyl}carbamoyl)-2-{[(2S,3R,4S,5R,6R)-3,4,5-trihydroxy-6-(hydroxymethyl)oxan-2-yl]oxy}phenoxy]sulfonyl}oxy)phenyl]-2,2-dimethylpentanoic Acid (LP9)

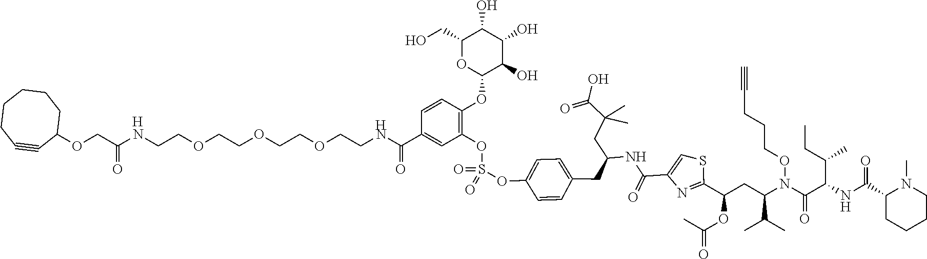

Following General Procedure IX using amine L4-10 (20 mg, 15 μmol) with OSu ester L0-Od (6.0 mg, 21 μmol), linker-payload LP9 (5.1 mg, 22% yield) was obtained as a white solid. ESI m/z: 772 (M/2+H)⁺. ¹H NMR (400 MHz, DMSO_{d6}) δ 8.75 (s, 1H), 8.45 (s, 3H), 8.20 (s, 1H), 8.00-7.90 (m, 2H), 7.55-7.50 (m, 1H), 7.45-7.30 (m, 3H), 7.25-7.20 (m, 1H), 5.85-5.80 (m, 1H), 5.40-5.35 (m, 1H), 4.75-4.70 (m, 2H), 4.50-4.35 (m, 5H), 4.30-4.25 (m, 3H), 4.20-4.00 (m, 4H), 3.85-3.75 (m, 4H), 3.65-3.60 (m, 4H), 2.75-2.60 (m, 3H), 2.60-2.50 (m, 3H), 2.40-2.30 (m, 2H), 2.20-1.95 (m, 14H), 1.90-1.60 (m, 14H), 1.50-1.20 (m, 16H), 1.10-0.90 (m, 9H), 0.85-0.80 (m, 6H), 0.70-0.60 (m, 3H) ppm.

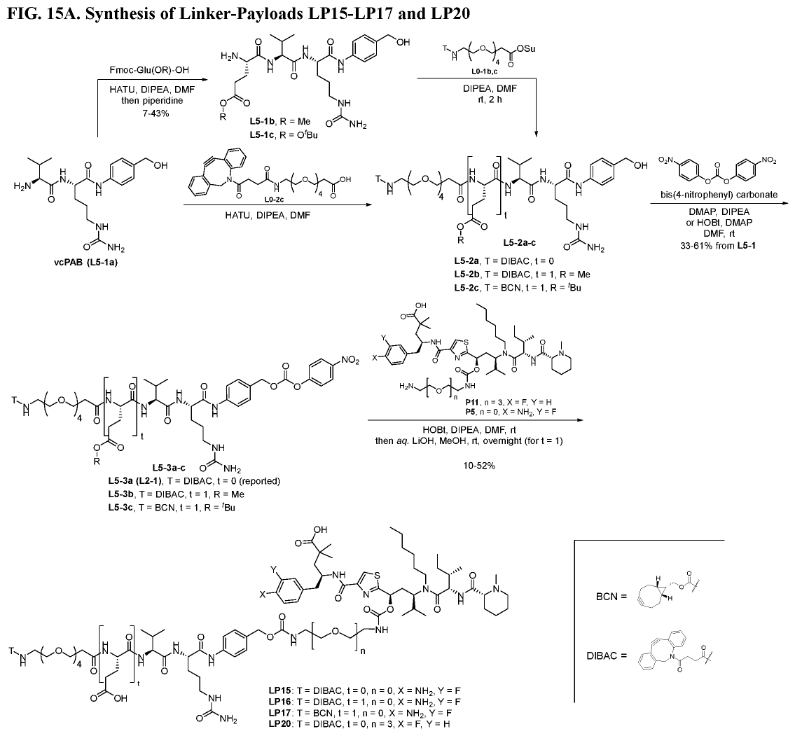

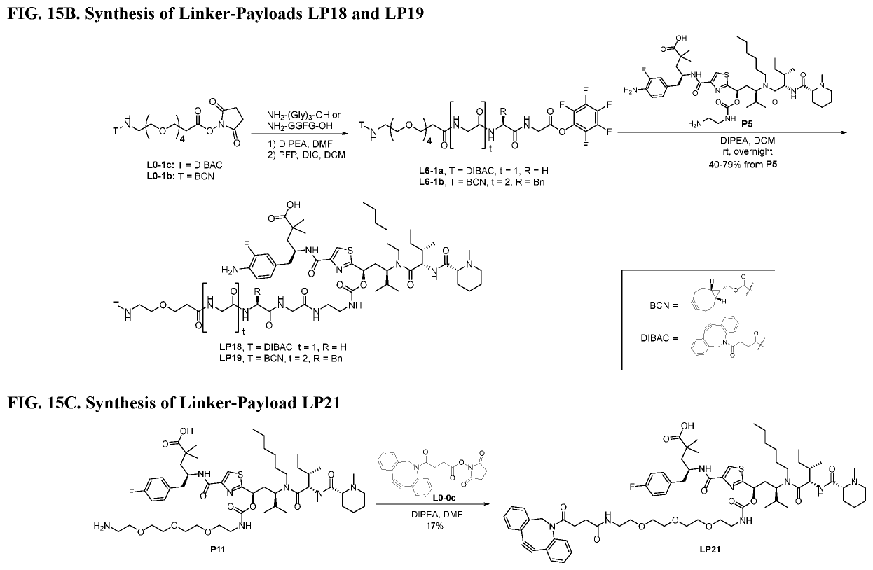

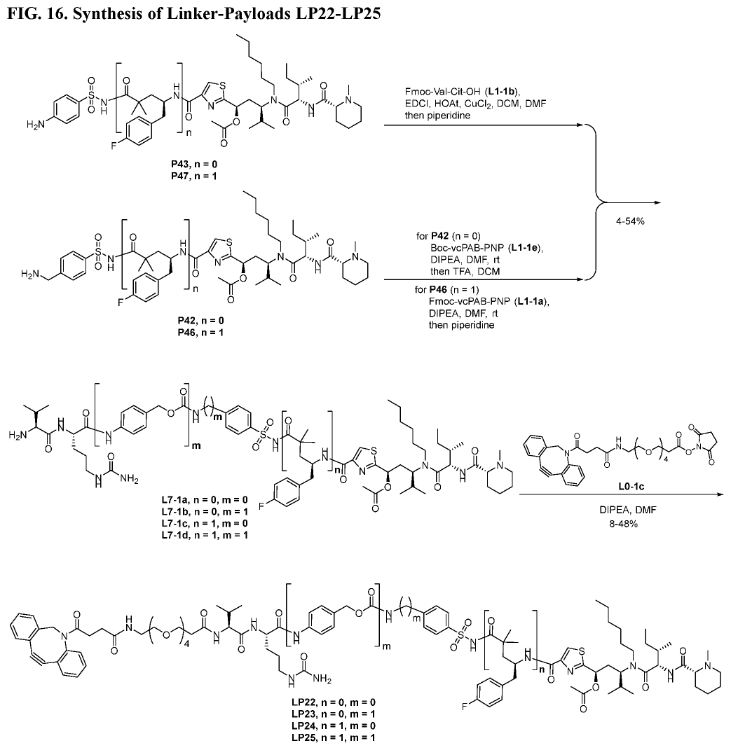

Synthesis of vcPAB-linker-tubulysins as in FIG. 15A.

### Methyl (4S)-4-amino-4-{[(1S)-1-{[(1S)-4-(carbamoylamino)-1-{[4-(hydroxymethyl)phenyl]carbamoyl}butyl]carbamoyl}-2-methylpropyl]carbamoyl}butanoate (L5-1b)

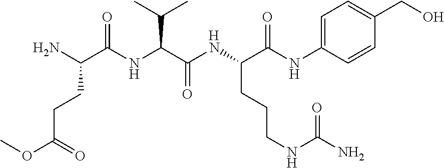

To a solution of Fmoc-Glu(OMe)-OH (0.30 g, 0.78 mmol) in DMF (10 mL) was added HATU (0.45 g, 1.2 mmol) and DIPEA (0.30 g, 2.3 mmol). The mixture was stirred at room temperature for 10 minutes before the addition of vcPAB (L5-1a) (0.30 g, 0.78 mmol). The reaction mixture was stirred at room temperature for 4 hours, and monitored by LCMS. The resulting mixture was diluted with DCM (200 mL). The organic solution was washed with water (100 mL) and brine (100 mL×2), dried over anhydrous sodium sulfate, and concentrated in vacuo. The residue was purified by reversed phase flash chromatography (0-100% acetonitrile in water) to give compound Fmoc-L5-lb (0.23 g, ESI m/z: 745 (M+H)⁺) as a white solid. To a solution of Fmoc-L5-lb (0.15 g) in DMF (5 mL) was added piperidine (86 mg, 1.0 mmol), and the mixture was stirred at room temperature for an hour until Fmoc was totally removed according to LCMS. The resulting mixture was directly purified by reversed phase flash chromatography (0-100% acetonitrile in aq. ammonium bicarbonate (0.05%)) to give Glu(OMe)-vcPAB (L5-1b) (20 mg, 7% yield) as a white solid. ESI m/z: 523 (M+H)⁺.

### tert-Butyl (4S)-4-amino-4-{[(1S)-1-{[(1S)-4-(carbamoylamino)-1-{[4-(hydroxymethyl)phenyl]carbamoyl}butyl]carbamoyl}-2-methylpropyl]carbamoyl}butanoate (L5-1c)

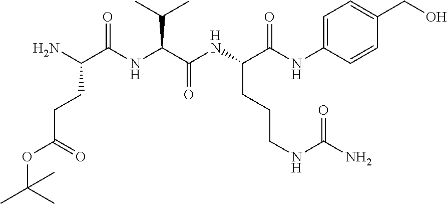

Following a similar procedure for L5-1b except using Fmoc-Glu(O^{t}Bu)-OH instead of Fmoc-Glu(OMe)-OH, compound L5-1c (0.12 g, 43% yield from vcPAB) was obtained as a white solid. ESI m/z: 565 (M+H)⁺. ¹H NMR (400 MHz, DMSO_{d6}) δ 10.00 (s, 1H), 8.42 (d, J=8.4 Hz, 1H), 8.31 (d, J=7.2 Hz, 1H), 8.13 (br s, 3H), 7.54 (d, J=8.4 Hz, 2H), 7.23 (d, J=8.4 Hz, 2H), 8.03 (t, J=5.6 Hz, 1H), 5.47 (s, 2H), 5.11 (br s, 1H), 4.45-4.42 (m, 3H), 4.26 (t, J=7.6 Hz, 1H), 3.92-3.86 (m, 1H), 3.10-3.01 (m, 1H), 2.96-2.89 (m, 1H), 2.34-2.30 (m, 2H), 2.03-1.98 (m, 1H), 1.94-1.88 (m, 2H), 1.74-1.65 (m, 1H), 1.62-1.53 (m, 1H), 1.48-1.32 (m, 10H), 0.91 (d, J=6.8 Hz, 3H), 0.88 (d, J=6.8 Hz, 3H) ppm.

### Methyl (4S)-4-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-4-{[(1S)-1-{[(1S)-4-(carbamoylamino)-1-[(4-{[(4-nitrophenoxycarbonyl)oxy]methyl}phenyl)carbamoyl]butyl]carbamoyl}-2-methylpropyl]carbamoyl}butanoate (L5-3b)

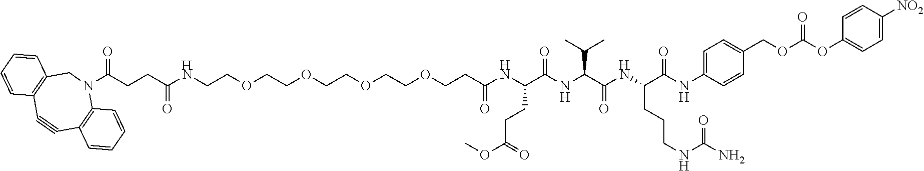

Following General Procedure IX using amine L5-1b (81 mg, 0.15 mmol) with OSu ester L0-1c, DIBAC-PEG4-Glu(OMe)-vcPAB (L5-2b) (94 mg, ESI m/z: 529.5 (M/2+H)⁺) was obtained as a white solid. vcPAB linker (20 mg) was dissolved in DMF (5 mL) and to the solution was added bis(4-nitrophenyl) carbonate (17 mg, 57 μmol) and DIPEA (0.01 mL). The mixture was stirred at room temperature for 24 hours, and monitored by LCMS. The resulting mixture was directly purified by reversed phase flash chromatography (0-100% acetonitrile in aq. ammonium bicarbonate (0.05%)) to give L5-3b (24 mg, 61% yield from L5-1b) as a yellow solid. ESI m/z: 612 (M/2+H)⁺.

### tert-Butyl (4S)-4-[1-({[endo-bicyclo[6.1.0]non-4-yn-9-ylmethoxy]carbonyl}amino)-3,6,9,12-tetraoxapentadecan-15-amido]-4-{[(1S)-1-{[(1S)-4-(carbamoylamino)-1-[(4-{[(4-nitrophenoxycarbonyl)oxy]methyl}phenyl)carbamoyl]butyl]carbamoyl}-2-methylpropyl]carbamoyl}butanoate (L5-3c)

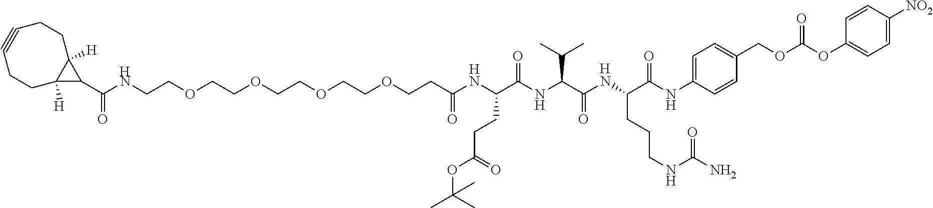

Following General Procedure IX using amine L5-1c (25 mg, 45 μmol) with L0-1b, BCN-PEG₄-Glu(O^{t}Bu)-Val-Cit-PAB (L5-2c) (29 mg, ESI m/z: 989 (M+H)⁺) was obtained as a white solid after purification by reversed phase flash chromatography (0-100% acetonitrile in aq. TFA (0.01%)). ¹H NMR (400 MHz, DMSO_{d6}) δ 9.95 (s, 1H), 8.15 (d, J=7.2 Hz, 1H), 8.09 (d, J=8.0 Hz, 1H), 7.74 (d, J=8.8 Hz, 1H), 7.54 (d, J=8.4 Hz, 2H), 7.23 (d, J=8.4 Hz, 2H), 7.12 (t, J=6.0 Hz, 1H), 6.00 (s, 1H), 5.44 (br s, 2H), 4.43 (s, 2H), 4.39-4.30 (m, 2H), 4.21-4.17 (m, 1H), 4.03 (d, J=8.0 Hz, 2H), 3.62-3.56 (m, 2H), 3.49-3.46 (m, 12H), 3.39 (t, J=5.6 Hz, 2H), 3.14-3.09 (m, 2H), 3.07-3.02 (m, 1H), 3.00-2.90 (m, 1H), 2.44-2.38 (m, 1H), 2.36-2.31 (m, 1H), 2.27-2.18 (m, 4H), 2.16-2.12 (m, 4H), 2.02-1.94 (m, 1H), 1.90-1.83 (m, 1H), 1.73-1.64 (m, 2H), 1.61-1.48 (m, 4H), 1.44-1.36 (m, 11H), 1.29-1.22 (m, 1H), 0.88-0.81 (m, 8H) ppm.

To a solution of L5-2c (29 mg) in dry DMF (3 mL) was subsequently added HOBt (8.0 mg, 58 μmol), DMAP (7.0 mg, 58 μmol), and bis(4-nitrophenyl) carbonate (18 mg, 58 mol). The reaction mixture was stirred at room temperature for 4 hours, and monitored by LCMS. The resulting mixture was purified directly by reversed phase flash chromatography (0-100% acetonitrile in water) to give L5-3c (17 mg, 33% yield from L5-1c) as a white solid. ¹H NMR (400 MHz, DMSO_{d6}) δ 10.12 (s, 1H), 8.32 (d, J=9.2 Hz, 2H), 8.19 (d, J=6.8 Hz, 1H), 8.09 (d, J=8.0 Hz, 1H), 7.74 (d, J=8.0 Hz, 1H), 7.65 (d, J=8.8 Hz, 2H), 7.57 (d, J=9.2 Hz, 2H), 7.41 (d, J=8.8 Hz, 2H), 7.12 (t, J=5.6 Hz, 1H), 6.00 (t, J=5.2 Hz, 1H), 5.45 (s, 2H), 5.25 (s, 2H), 4.42-4.30 (m, 2H), 4.22-4.18 (m, 1H), 4.03 (d, J=8.0 Hz, 2H), 3.61-3.56 (m, 2H), 3.49-3.48 (m, 12H), 3.39 (t, J=6.0 Hz, 2H), 3.13-3.09 (m, 2H), 3.06-3.02 (m, 1H), 2.98-2.91 (m, 1H), 2.46-2.38 (m, 1H), 2.35-2.31 (m, 1H), 2.23-2.12 (m, 8H), 2.02-1.95 (m, 1H), 1.91-1.83 (m, 1H), 1.73-1.65 (m, 2H), 1.61-1.42 (m, 4H), 1.38 (s, 9H), 1.28-1.19 (m, 2H), 0.87-0.81 (m, 8H) ppm.

### LP15: (4S)-5-(4-amino-3-fluorophenyl)-4-({2-[(1R,3R)-1-{[(2-{[({4-[(2S)-2-[(2S)-2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methoxy)carbonyl]amino}ethyl)carbamoyl]oxy}-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic Acid (LP15)

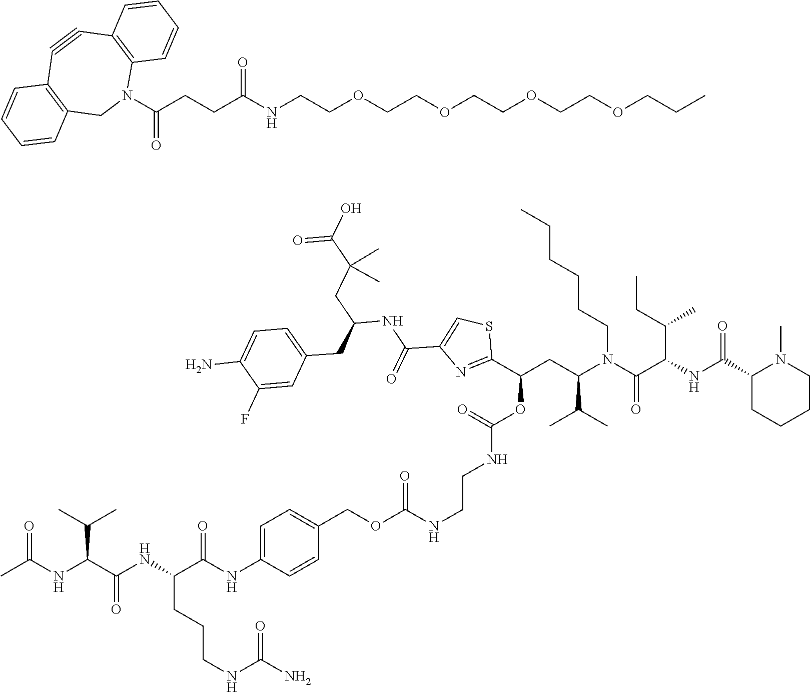

Following General Procedure X using PNP ester L5-3a with amine P5, linker-payload LP15 (6 mg with 95% purity; and 4 mg with 88% purity, 36% yield) was obtained as a white solid. ESI m/z: 611 (M/3+H)⁺, 915 (M/2+H)⁺. ¹H NMR (400 MHz, DMSO_{d6}) δ 12.18 (s, 1H), 10.00 (s, 1H), 8.19 (br s, 2H), 7.88 (d, J=8.0 Hz, 1H), 7.77 (t, J=5.6 Hz, 1H), 7.69-7.59 (m, 6H), 7.52-7.31 (m, 6H), 7.31-7.21 (m, 3H), 7.22-7.18 (m, 1H), 6.75 (d, J=12.8 Hz, 1H), 6.68-6.61 (m, 2H), 5.99 (t, J=5.2 Hz, 1H), 5.58-5.54 (m, 1H), 5.42 (s, 2H), 5.03 (d, J=14.0 Hz, 1H), 4.95-4.93 (m, 4H), 4.48 (t, J=9.2 Hz, 1H), 4.41-4.35 (m, 1H), 4.24-4.21 (m, 2H), 3.73 (br s, 1H), 3.63-3.56 (m, 3H), 3.48-3.45 (m, 14H), 3.30-3.28 (m, 2H), 3.09-2.91 (m, 9H), 2.85-2.81 (m, 1H), 2.62-2.54 (m, 3H), 2.48-2.44 (m, 1H), 2.40-2.13 (m, 3H), 2.08 (br s, 1H), 2.04 (s, 3H), 2.00-1.66 (m, 10H), 1.62-1.34 (m, 11H), 1.27-1.24 (m, 6H), 1.12 (d, J=6.8 Hz, 1H), 1.07-1.06 (m, 6H), 0.95 (d, J=6.4 Hz, 3H), 0.87-0.80 (m, 15H), 0.70 (br s, 3H) ppm.

### LP16: (4S)-5-(4-amino-3-fluorophenyl)-4-({2-[(1R,3R)-1-{[(2-{[({4-[(2S)-2-[(2S)-2-[(2S)-2-[1-(4-{2-azatricyclo [10.4.0.0^{4,9}]hexadeca-1(12),4(9),5,7,13,15-hexaen-10-yn-2-yl}-4-oxobutanamido)-3,6,9,12-tetraoxapentadecan-15-amido]-4-carboxybutanamido]-3-methylbutanamido]-5-(carbamoylamino)pentanamido]phenyl}methoxy)carbonyl]amino}ethyl)carbamoyl]oxy}-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic acid (LP16)

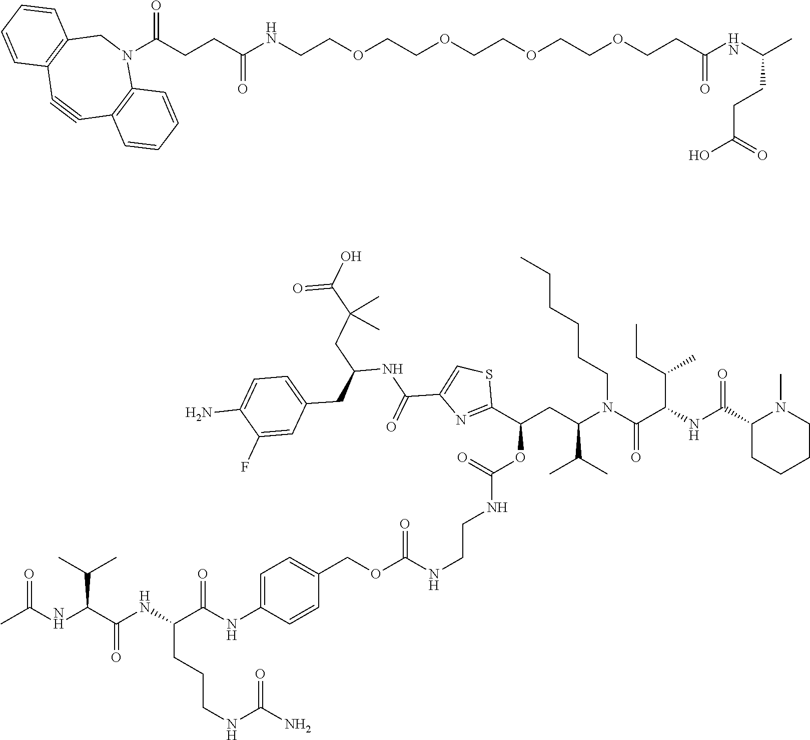

Following General Procedure X using PNP ester L5-3b with amine P5 (10 mg, 12 μmol), a solution of linker-payload LP16-OMe (ESI m/z: 658 (M/3+H)⁺) in DMF was obtained. To this solution was added methanol (5 mL) and aq. lithium hydroxide (3.0 mL, 10 mM). The reaction mixture was stirred at room temperature overnight, and monitored by LCMS. The resulting mixture was purified directly by reversed phase flash chromatography (0-100% acetonitrile in aq. ammonium bicarbonate (10 mM)) to give linker-payload LP16 (4.0 mg, 10% yield from P5) as a white solid. ESI m/z: 653 (M/3+H)⁺, 980 (M/2+H)⁺. ¹H NMR (400 MHz, methanol_{d4}) δ 7.95 (s, 1H), 7.54-7.52 (m, 3H), 7.49-7.47 (m, 1H), 7.36-7.33 (m, 3H), 7.27-7.19 (m, 4H), 7.15-7.13 (m, 1H), 7.72 (d, J=12.4 Hz, 1H), 6.68-6.60 (m, 2H), 5.53-5.50 (m, 1H), 5.02 (d, J=14.4 Hz, 2H), 4.92 (s, 2H), 4.55 (d, J=10.8 Hz, 1H), 4.47 (br s, 9H), 4.33-3.44 (m, 13H), 3.32-3.30 (m, 1H), 3.14-3.03 (m, 10H), 2.60-2.56 (m, 2H), 2.37-2.24 (m, 8H), 2.10-2.03 (m, 2H), 1.98-1.80 (m, 8H), 1.70-1.63 (m, 2H), 1.59-1.46 (m, 7H), 1.30-1.21 (m, 7H), 1.18 (s, 2H), 1.10-1.00 (m, 7H), 0.91-0.87 (m, 13H), 0.81-0.74 (m, 12H) ppm.

### LP17: (4S)-5-(4-amino-3-fluorophenyl)-4-({2-[(1R,3R)-1-{[(2-{[({4-[(2S)-2-[(2S)-2-[(2S)-2-j[1-({[endo-bicyclo[6.1.0]non-4-yn-9-ylmethoxy]carbonyl}amino)-3,6,9,12-tetraoxapentadecan-15-amido]-4-carboxybutanamido]-3-methylbutanamido]-5-(carbamoylamino)pentanamido]phenyl}methoxy)carbonyl]amino}ethyl)carbamoyl]oxy}-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic Acid (LP17)

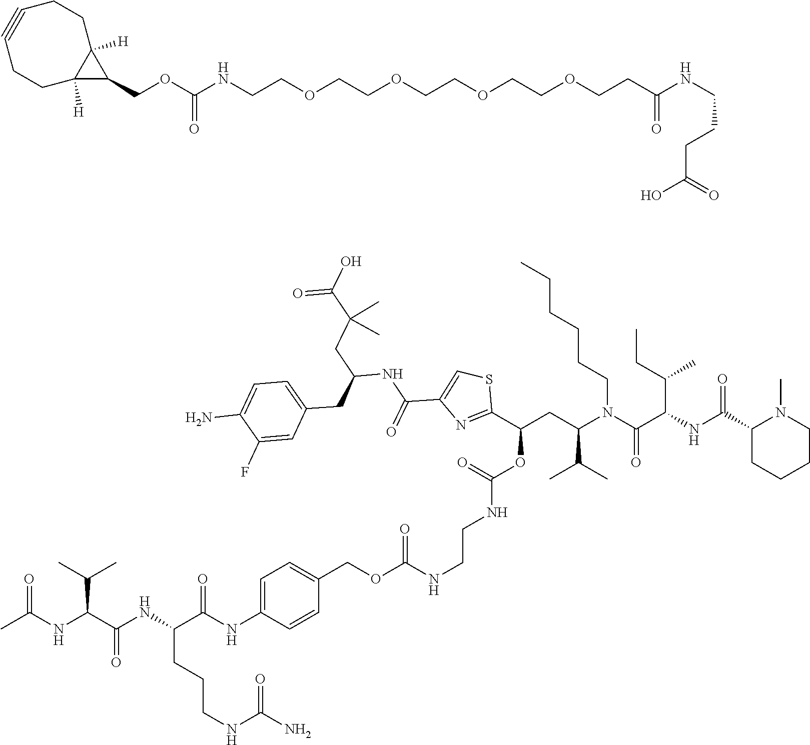

Following General Procedure X using PNP ester L5-3c with amine P5 (13 mg, 11 mol), linker-payload LP17-O^{t}Bu (10 mg, ESI m/z: 953 (M/2+H)⁺) was obtained as a white solid after purification by reversed phase flash chromatography (0-100% acetonitrile in water). To a solution of LP17-O^{t}Bu (7.0 mg, 3.7 μmol) in THE (1.8 mL) was added aq. lithium hydroxide (0.6 mL, 2 M). The mixture was stirred at room temperature overnight, and monitored by LCMS. The resulting mixture was concentrated in vacuo to remove THF, and the residual aqueous mixture was neutralized with aq. TFA (2 M) to pH 7.0 at 0° C. The mixture was purified by prep-HPLC (0-100% acetonitrile in aq. ammonium bicarbonate (10 mM)) to give linker-payload LP17 (2.0 mg, 14% yield from P5) as a white solid. ESI m/z: 925 (M/2+H)⁺. ¹H NMR (400 MHz, DMSO_{d6}) δ 10.06 (s, 1H), 8.24-8.21 (m, 1H), 8.13-8.09 (m, 2H), 7.75 (d, J=8.4 Hz, 1H), 7.66 (t, J=5.6 Hz, 1H), 7.58 (d, J=8.4 Hz, 2H), 7.28 (d, J=8.4 Hz, 2H), 7.25-7.21 (m, 1H), 7.14-7.11 (m, 1H), 6.75 (d, J=12.0 Hz, 1H), 6.68-6.60 (m, 2H), 8.05-5.98 (m, 1H), 5.59-5.53 (m, 1H), 5.46 (s, 2H), 5.02-4.90 (m, 4H), 4.50-4.44 (m, 1H), 4.37-4.31 (m, 2H), 4.24-4.17 (m, 2H), 4.03 (d, J=8.0 Hz, 2H), 3.77-3.69 (m, 1H), 3.61-3.56 (m, 2H), 3.49-3.47 (m, 12H), 3.13-3.02 (m, 10H), 2.86-2.82 (m, 1H), 2.61-2.59 (m, 1H), 2.44-2.29 (m, 2H), 2.25-2.08 (m, 12H), 2.03-1.94 (m, 3H), 1.93-1.82 (m, 5H), 1.73-1.36 (m, 17H), 1.33-1.23 (m, 12H), 1.18-1.06 (m, 7H), 0.95 (d, J=6.0 Hz, 3H), 0.85-0.78 (m, 17H), 0.72-0.64 (m, 3H) ppm. ¹⁹F NMR (376 MHz, DMSO_{d6}) δ −135 ppm.

### LP20: (4S)-4-({2-[(1R,3R)-1-{[(2-{2-[2-(2-{[({4-[(2S)-2-[(2S)-2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methoxy)carbonyl]amino}ethoxy)ethoxy]ethoxy}ethyl)carbamoyl]oxy}-3-[(2S,3S)—N-hexyl-3-methyl-2-{[(2R)-1-methylpiperidin-2-yl]formamido}pentanamido]-4-methylpentyl]-1,3-thiazol-4-yl}formamido)-5-(4-fluorophenyl)-2,2-dimethylpentanoic Acid (LP20)

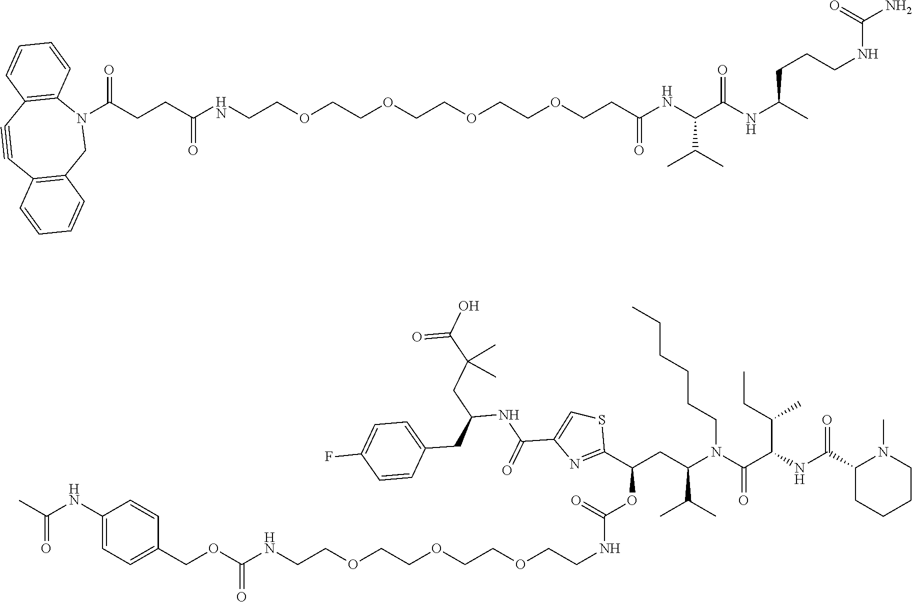 [SMILES: CC(C)[C@H](NC(=O)CCOCCOCCOCCOCCNC(=O)CCC(=O)N1Cc2ccccc2C#Cc2ccccc21)C(=O)N[C@H](C)CCCNC(N)=O ; CCCCCCN(C(=O)[C@@H](NC(=O)[C@H]1CCCCN1C)[C@@H](C)CC)[C@H](C[C@@H](OC(=O)NCCOCCOCCOCCNC(=O)OCc1ccc(NC(C)=O)cc1)c1nc(C(=O)N[C@@H](Cc2ccc(F)cc2)CC(C)(C)C(=O)O)cs1)C(C)C]

Following General Procedure X using PNP ester L5-3a with amine P11 (11 mg, 9.8 mol, TFA salt), linker-payload LP20 (10 mg, 52% yield) was obtained as a white solid. ESI m/z: 649 (M/3+H)⁺, 974 (M/2+H)⁺. ¹H NMR (400 MHz, DMSO_{d6}) δ 10.02 (s, 1H), 8.17-8.16 (m, 1H), 8.13 (s, 1H), 7.90 (d, J=8.4 Hz, 1H), 7.77 (t, J=5.6 Hz, 1H), 7.69-7.67 (m, 2H), 7.63-7.55 (m, 4H), 7.52-7.45 (m, 3H), 7.40-7.32 (m, 2H), 7.31-7.26 (m, 3H), 7.23-7.17 (m, 3H), 7.06 (t, J=8.8 Hz, 2H), 6.02-5.99 (m, 1H), 5.58-5.54 (m, 1H), 5.43 (s, 2H), 5.33 (t, J=4.8 Hz, 1H), 5.03 (d, J=14.0 Hz, 1H), 4.98-4.93 (br s, 2H), 4.48 (t, J=9.6 Hz, 1H), 4.41-4.35 (m, 1H), 4.31-4.21 (m, 2H), 3.63-3.57 (m, 3H), 3.50-3.45 (m, 22H), 3.30-3.28 (m, 1H), 3.15-3.07 (m, 4H), 3.01-2.92 (m, 3H), 2.85-2.74 (m, 3H), 2.60-2.55 (m, 1H), 2.46-2.33 (m, 2H), 2.26-2.20 (m, 1H), 2.16-2.12 (m, 1H), 2.08 (s, 3H), 2.03-1.94 (m, 5H), 1.88-1.84 (m, 2H), 1.80-1.72 (m, 2H), 1.69-1.65 (m, 2H), 1.60-1.57 (m, 3H), 1.51-1.44 (m, 3H), 1.40-1.33 (m, 2H), 1.28-1.23 (m, 15H), 1.16-1.11 (m, 2H), 1.07 (s, 3H), 1.06 (s, 3H), 0.95 (d, J=6.4 Hz, 3H), 0.87-0.79 (m, 16H), 0.71 (br s, 3H) ppm. ¹⁹F NMR (376 MHz, DMSO_{d6}) δ −117 ppm.

Synthesis of Linker-tubulysin via Carbamates as in FIG. 15B.

### LP18: (4S)-5-(4-amino-3-fluorophenyl)-4-({2-[(1R,3R)-1-[({2-[2-(2-{2-[1-(4-{2-azatricyclo[10.4.0.0_{4,9}]hexadeca-1(12),4(9),5,7,13,15-hexaen-10-yn-2-yl}-4-oxobutanamido)-3,6,9,12-tetraoxapentadecan-15-amido]acetamido}acetamido)acetamido]ethyl}carbamoyl)oxy]-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic Acid (LP18)

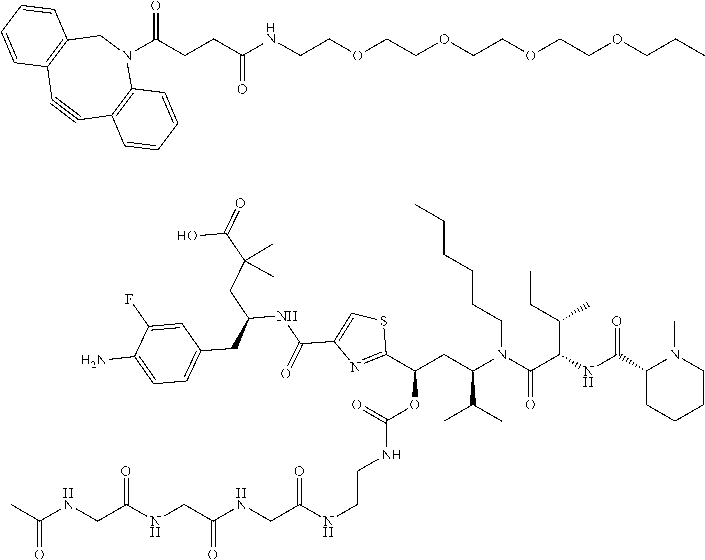

Following General Procedure IX using OSu ester L0-1c (1.0 g, 1.5 mmol) with H-Gly-Gly-Gly-OH, crude linker DIBAC-PEG4-Gly-Gly-Gly-OH (0.90 g, ESI m/z: 734 (M+H)*) was obtained as a white solid, and used in the next step without further purification. To a solution of the linker (10 mg) in dry DCM (5.0 mL) was added pentafluorophenol (5.1 mg, 28 mol) and DIC (5.2 mg, 41 μmol). The reaction mixture was stirred at room temperature for an hour, and monitored by LCMS. The volatiles were removed in vacuo to give crude ester L6-1a (16 mg, ESI m/z: 890 (M+H)⁺), which was added to a mixture of P5 (7.0 mg, 7.9 μmol) and DIPEA (3.1 mg, 24 μmol) in DCM (5.0 mL). The mixture was stirred at room temperature for half an hour, and monitored by LCMS. The resulting mixture was concentrated in vacuo and the residue was purified by prep-HPLC (0-100% acetonitrile in aq. ammonium bicarbonate (10 mM)) to give linker-payload LP18 (10 mg, 79% yield from P5) as a white solid. ESI m/z: 798 (M/2+H)⁺. ¹H NMR (400 MHz, methanol_{d4}) δ 7.98 (s, 1H), 7.54 (d, J=6.8 Hz, 1H), 7.50-7.48 (m, 1H), 7.37-7.34 (m, 3H), 7.27-7.20 (m, 2H), 7.15-7.13 (m, 1H), 6.74-6.60 (m, 3H), 5.55 (d, J=12.4 Hz, 1H), 5.03 (d, J=14.0 Hz, 1H), 4.57-4.45 (m, 6H), 4.22 (br s, 1H), 3.80-3.75 (m, 5H), 3.65-3.58 (m, 3H), 3.49 (s, 8H), 3.45-3.43 (m, 2H), 3.34-3.30 (m, 2H), 3.16-3.08 (m, 4H), 2.88-2.86 (m, 1H), 2.67-2.55 (m, 4H), 2.42 (t, J=6.0 Hz, 2H), 2.29-2.22 (m, 1H), 2.17-2.03 (m, 6H), 1.94-1.83 (m, 4H), 1.74-1.70 (m, 2H), 1.60-1.42 (m, 7H), 1.26-1.23 (m, 9H), 1.18-1.15 (m, 2H), 1.05 (s, 3H), 1.01 (s, 3H), 0.92-0.87 (m, 6H), 0.82-0.79 (m, 7H), 0.74-0.70 (m, 3H) ppm. ¹⁹F NMR (376 MHz, DMSO_{d6}) δ −137 ppm.

### LP19: (4S)-5-(4-amino-3-fluorophenyl)-4-({2-[(1R,3R)-1-{[(2-{2-[(2S)-2-(2-{2-[1-({[endo-bicyclo[6.1.0]non-4-yn-9-ylmethoxy]carbonyl}amino)-3,6,9,12-tetraoxapentadecan-15-amido]acetamido}acetamido)-3-phenylpropanamido]acetamido}ethyl)carbamoyl]oxy}-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic Acid (LP19)

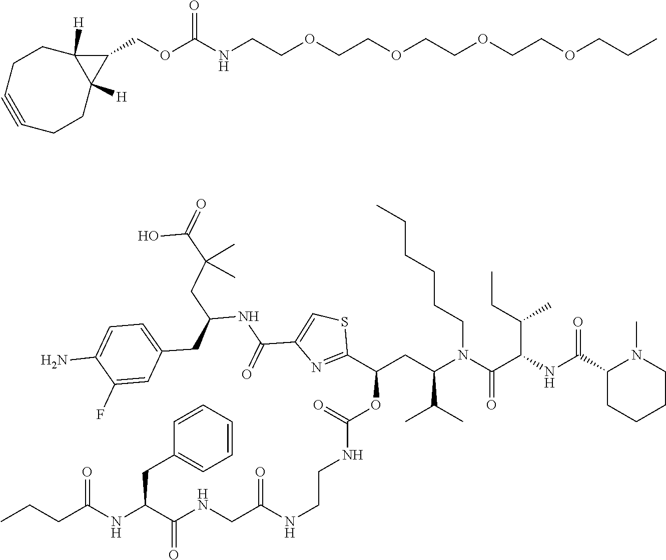

Following a similar procedure for LP18 except starting from OSu ester L0-1b instead of L0-1c, linker-payload LP19 (12 mg, TFA salt, 40% yield from P5) was obtained as a white solid after purification by prep-HPLC (0-100% acetonitrile in aq. TFA (0.01%)). ESI m/z. 816 (M/2+H)⁺. ¹H NMR (400 MHz, DMSO_{d6}) δ 8.36 (s, 1H), 8.32-8.27 (m, 1H), 8.23-8.19 (m, 1H), 8.17-8.13 (m, 2H), 8.10-8.04 (m, 1H), 7.83-7.79 (m, 1H), 7.78-7.69 (m, 1H), 7.66-7.61 (m, 1H), 7.53-7.42 (m, 1H), 7.28-7.24 (m, 4H), 7.21-7.17 (m, 1H), 7.12 (t, J=2.4 Hz, 1H), 6.75 (d, J=12.0 Hz, 1H), 6.68-6.60 (m, 2H), 5.59-5.54 (m, 1H), 4.94 (s, 2H), 4.53-4.45 (m, 2H), 4.27-4.18 (m, 1H), 4.03 (d, J=8.0 Hz, 2H), 3.79-3.68 (m, 7H), 3.62-3.58 (m, 4H), 3.49-3.47 (m, 14H), 3.15-3.10 (m, 3H), 3.09-3.05 (m, 4H), 2.84-2.78 (m, 2H), 2.61-2.60 (m, 2H), 2.40 (d, J=6.4 Hz, 2H), 2.26-2.15 (m, 8H), 2.07 (s, 3H), 1.98-1.76 (m, 5H), 1.67-1.45 (m, 8H), 1.38-1.34 (m, 2H), 1.28-1.24 (m, 8H), 1.17-1.10 (m, 1H), 1.06 (s, 3H), 1.05 (s, 3H), 0.94 (d, J=5.6 Hz, 3H), 0.85-0.79 (m, 11H), 0.69-0.65 (m, 3H) ppm. 19F NMR (376 MHz, DMSO_{d6}) δ −135 (Ar—F), −73.0 (CF₃CO₂H) ppm.

Synthesis of Linker-tubulysin LP21 as in FIG. 15C.

### LP21: (4S)-4-({2-[(1R,3R)-1-({[2-(2-{2-[2-(4-{2-azatricyclo[10.4.0.0^{4,9}]hexadeca-1(12),4(9),5,7,13,15-hexaen-10-yn-2-yl}-4-oxobutanamido)ethoxy]ethoxy}ethoxy)ethyl]carbamoyl}oxy)-3-[(2S,3S)—N-hexyl-3-methyl-2-{[(2R)-1-methylpiperidin-2-yl]formamido}pentanamido]-4-methylpentyl]-1,3-thiazol-4-yl}formamido)-5-(4-fluorophenyl)-2,2-dimethylpentanoic Acid (LP21)

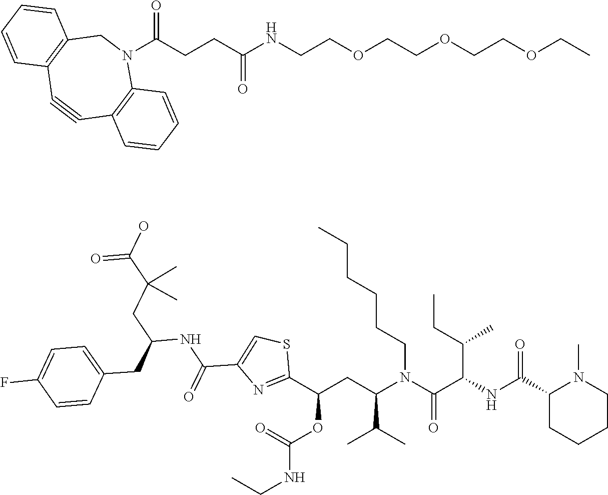 [SMILES: CCOCCOCCOCCNC(=O)CCC(=O)N1Cc2ccccc2C#Cc2ccccc21 ; CCCCCCN(C(=O)[C@@H](NC(=O)[C@H]1CCCCN1C)[C@@H](C)CC)[C@H](C[C@@H](OC(=O)NCC)c1nc(C(=O)N[C@@H](Cc2ccc(F)cc2)CC(C)(C)C(=O)O)cs1)C(C)C]

Following General Procedure IX using amine P11 (5.0 mg, 4.9 μmol) with OSu ester L0-0c (2.0 mg, 4.9 μmol), compound LP21 (1.1 mg, 17% yield) was obtained as a white solid. ESI m/z: 647 (M/2+H). ¹H NMR (400 MHz, DMSO_{d6}) δ 8.13 (s, 1H), 7.76 (t, J=5.6 Hz, 1H), 7.69-7.67 (m, 1H), 7.64-7.61 (m, 1H), 7.56 (t, J=6.0 Hz, 1H), 7.52-7.45 (m, 3H), 7.38-7.34 (m, 2H), 7.30-7.28 (m, 1H), 7.21-7.17 (m, 2H), 7.07 (t, J=9.2 Hz, 2H), 5.58-5.54 (m, 1H), 5.03 (d, J=13.6 Hz, 1H), 4.48 (t, J=9.2 Hz, 1H), 4.28 (br s, 1H), 3.74-3.67 (m, 1H), 3.61 (d, J=13.6 Hz, 1H), 3.49-3.43 (m, 10H), 3.11-3.07 (m, 4H), 3.00-2.92 (m, 2H), 2.86-2.76 (m, 3H), 2.62-2.56 (m, 1H), 2.28-2.11 (m, 3H), 2.08 (s, 3H), 2.03-1.95 (m, 2H), 1.91-1.85 (m, 2H), 1.80-1.70 (m, 3H), 1.63-1.49 (m, 5H), 1.41-1.24 (m, 15H), 1.07 (s, 3H), 1.06 (s, 3H), 0.95 (d, J=6.4 Hz, 3H), 0.88-0.79 (m, 10H), 0.70 (br s, 3H) ppm. ¹⁹F NMR (376 MHz, DMSO_{d6}) δ −117 ppm.

Synthesis of Linker-N-acylsulfonamide-tubulysins as in FIG. 16.

### (1R,3R)-1-[4-({4-[(2S)-2-[(2S)-2-Amino-3-methylbutanamido]-5-(carbamoylamino)pentanamido]benzenesulfonyl}carbamoyl)-1,3-thiazol-2-yl]-3-[(2S,3S)—N-hexyl-3-methyl-2-{[(2R)-1-methylpiperidin-2-yl]formamido}pentanamido]-4-methylpentyl Acetate (L7-1a)

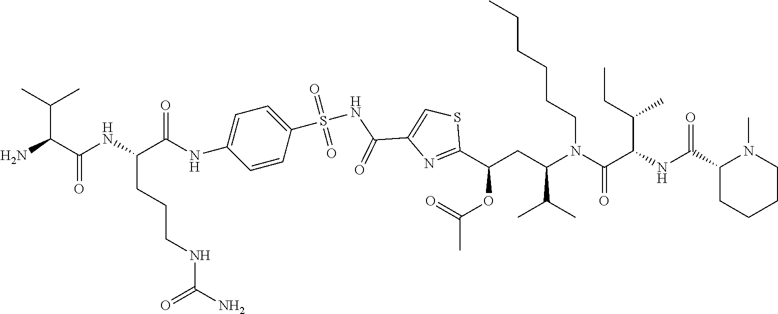 [SMILES: CCCCCCN(C(=O)[C@@H](NC(=O)[C@H]1CCCCN1C)[C@@H](C)CC)[C@H](C[C@@H](OC(C)=O)c1nc(C(=O)NS(=O)(=O)c2ccc(NC(=O)[C@H](CCCNC(N)=O)NC(=O)[C@@H](N)C(C)C)cc2)cs1)C(C)C]

To a solution of Fmoc-Val-Cit-OH (49 mg, 98 μmol) in DMF (0.5 mL) and DCM (4 mL) was added HOAt (14 mg, 98 μmol) and EDCI (19 mg, 98 μmol). The mixture was stirred at room temperature for 15 minutes before the addition of payload P43 (25 mg, 33 mol) and copper(II) chloride (17 mg, 98 μmol). The reaction mixture was stirred at room temperature for 55 hours, and monitored by LCMS. The resulting mixture was filtered and the filtrate was concentrated in vacuo. The residue was purified by reversed phase flash chromatography (0-30% acetonitrile in aq. ammonium bicarbonate (10 mM)) to give compound Fmoc-L7-la (20 mg, ESI m/z: 621 (M/2+H)⁺) as a white solid. Fmoc-L7-la was dissolved in DMF (1 mL). To the solution was added piperidine (6.0 mg, 64 μmol), and the mixture was stirred at room temperature for 2 hours until Fmoc was totally removed according to LCMS. The resulting mixture was purified directly by reversed phase flash chromatography (5-50% acetonitrile in aq. ammonium bicarbonate (10 mM)) to give L7-1a (9.0 mg, 27% yield from P43) as a white solid. ESI m/z: 510 (M/2+H)⁺.

### LP22: (1R,3R)-1-[4-({4-[(2S)-2-[(2S)-2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]benzenesulfonyl}carbamoyl)-1,3-thiazol-2-yl]-3-[(2S,3S)—N-hexyl-3-methyl-2-{[(2R)-1-methylpiperidin-2-yl]formamido}pentanamido]-4-methylpentyl Acetate (LP22)

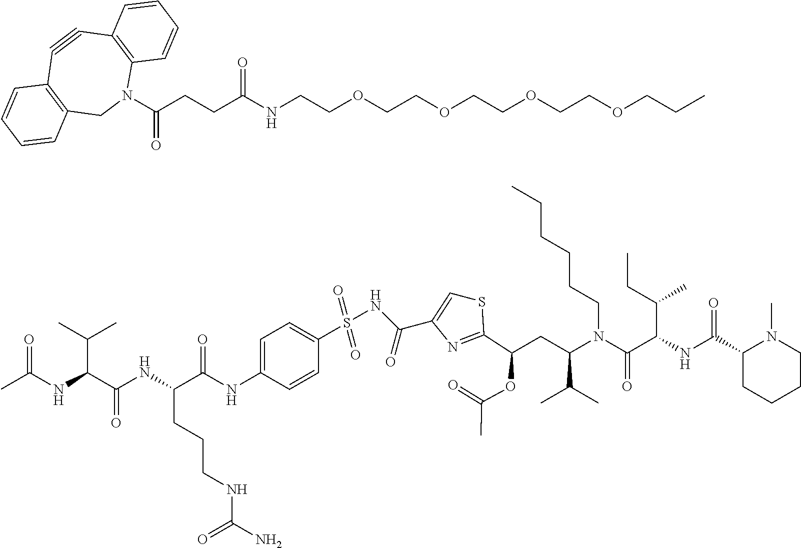

Following General Procedure IX using amine L7-1a (9.0 mg, 8.8 μmol) with OSu ester L0-1c, linker-payload LP22 (1.1 mg, 8% yield) was obtained as a white solid. ESI m/z: 777 (M/2+H)⁺. ¹H NMR (500 MHz, DMSO_{d6}) δ 10.10 (s, 1H), 8.15-8.13 (d, J=7.6 Hz, 1H), 7.92 (s, 1H), 7.87-7.85 (d, J=7.6 Hz, 1H), 7.78-7.72 (m, 4H), 7.71-7.56 (m, 5H), 7.52-7.44 (m, 3H), 7.40-7.28 (m, 3H), 6.00-5.95 (m, 1H), 5.54-5.51 (m, 1H), 5.40 (s, 2H), 5.03 (d, J=14.0 Hz, 1H), 4.54-4.47 (m, 1H), 4.44-4.36 (m, 1H), 4.26-4.21 (t, J=8.0 Hz, 2H), 3.65 (s, 1H), 3.61-3.57 (m, 3H), 3.50-3.33 (m, 13H), 3.11-3.05 (m, 2H), 3.03-3.00 (m, 1H), 2.96-2.91 (m, 1H), 2.60-2.55 (m, 1H), 2.35-2.32 (m, 2H), 2.28-2.20 (m, 2H), 2.06 (s, 3H), 2.03-1.95 (m, 5H), 1.81-1.75 (m, 1H), 1.75-1.65 (m, 4H), 1.62-1.55 (m, 2H), 1.48-1.38 (m, 6H), 1.30-1.28 (m, 5H), 1.21-1.25 (m, 6H), 0.93-0.91 (d, J=6.8 Hz, 3H), 0.88-0.78 (m, 23H) ppm.

### (1R,3R)-1-(4-{[4-({[({4-[(2S)-2-[(2S)-2-Amino-3-methylbutanamido]-5-(carbamoylamino)pentanamido]phenyl}methoxy)carbonyl]amino}methyl)benzenesulfonyl]carbamoyl}-1,3-thiazol-2-yl)-3-[(2S,3S)—N-hexyl-3-methyl-2-{[(2R)-1-methylpiperidin-2-yl]formamido}pentanamido]-4-methylpentyl Acetate (L7-1b)

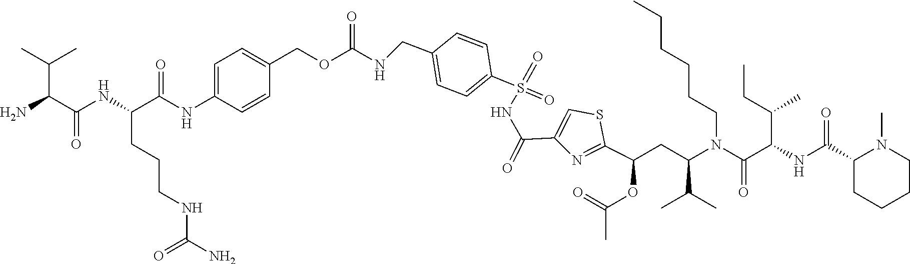 [SMILES: CCCCCCN(C(=O)[C@@H](NC(=O)[C@H]1CCCCN1C)[C@@H](C)CC)[C@H](C[C@@H](OC(C)=O)c1nc(C(=O)NS(=O)(=O)c2ccc(CNC(=O)OCc3ccc(NC(=O)[C@H](CCCNC(N)=O)NC(=O)[C@@H](N)C(C)C)cc3)cc2)cs1)C(C)C]

Following General Procedure X using Boc-vcPAB-PNP (L1-1e) with amine P42, compound Boc-L7-lb (15 mg, ESI m/z: 642 (M/2+H)⁺) was obtained as a white solid. Boc-L7-1b was dissolved in DCM (4.5 mL). To the solution was added TFA (0.5 mL), and the mixture was stirred at room temperature for 2 hours until Boc was totally removed according to LCMS. The resulting solution was concentrated in vacuo to give crude L7-1b (15 mg, contaminated with P42). Crude L7-1b was used in the next step without further purification. ESI m/z: 592 (M/2+H)⁺. ¹H NMR (400 MHz, DMSO_{d6}) of Boc-L7-lb (rotamers): δ 10.08 (s, 0.5H), 9.91 (s, 0.5H), 8.24 (d, J=7.6 Hz, 0.5H), 8.11 (dd, J=6.8 and 1.2 Hz, 1H), 7.99 (d, J=7.6 Hz, 0.5H), 7.91 (s, 1H), 7.84-7.80 (m, 1H), 7.74 (d, J=8.4 Hz, 2H), 7.64-7.57 (m, 2H), 7.30 (d, J=8.0 Hz, 2H), 7.23 (d, J=8.4 Hz, 2H), 6.80-6.75 (m, 1H), 6.00-5.95 (m, 1H), 5.89-5.81 (m, 1H), 5.58-5.52 (m, 1H), 5.41 (s, 2H), 4.97 (s, 2H), 4.49 (t, J=9.6 Hz, 1H), 4.48-4.37 (m, 1H), 4.20 (d, J=5.6 Hz, 2H), 4.01-3.94 (m, 1H), 3.85-3.81 (m, 1H), 3.03-2.93 (m, 7H), 2.33-2.32 (m, 3H), 2.23-2.18 (m, 2H), 2.06 (s, 3H), 1.98-1.83 (m, 3H), 1.74-1.44 (m, 7H), 1.39-1.36 (m, 10H), 1.29 (s, 6H), 1.24 (s, 2H), 1.12-1.05 (m, 1H), 1.00-0.95 (m, 2H), 0.93 (d, J=6.4 Hz, 3H), 0.86-0.79 (m, 16H), 0.74-0.68 (m, 3H) ppm.

LP23: (1R,3R)-1-(4-{[4-({[({4-[(2S)-2-[(2S)-2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methoxy)carbonyl]amino}methyl)benzenesulfonyl]carbamoyl}-1,3-thiazol-2-yl)-3-[(2S,3S)—N-hexyl-3-methyl-2-{[(2R)-1-methylpiperidin-2-yl]formamido}pentanamido]-4-methylpentyl acetate (LP23)

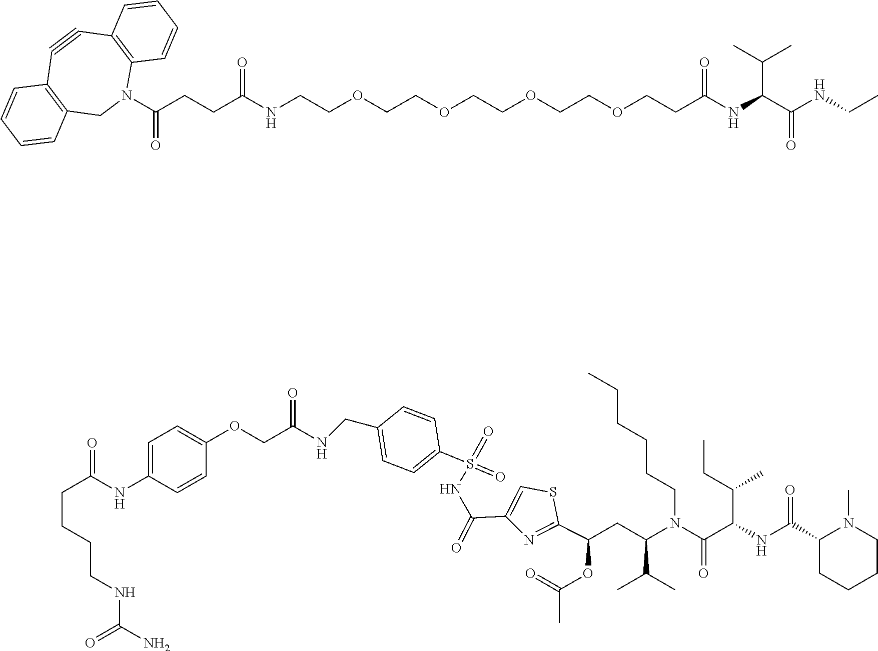

Following General Procedure IX using amine L7-1b with OSu ester L0-1c, linker-payload LP23 (2 mg, 13% yield from P42) was obtained as a white solid. ESI m/z: 573 (M/3+H)⁺, 859 (M/2+H)⁺. ¹H NMR (400 MHz, DMSO_{d6}) (rotamers) δ 10.00 (s, 0.3H), 9.92 (s, 0.7H), 8.40 (d, J=8.0 Hz, 0.7H), 8.13 (d, J=6.8 Hz, 0.3H), 8.00-7.82 (m, 3H), 7.78-7.74 (m, 3H), 7.69-7.58 (m, 4H), 7.52-7.43 (m, 3H), 7.40-7.29 (m, 5H), 7.23 (d, J=8.0 Hz, 2H), 6.00-5.96 (m, 1H), 5.53 (d, J=12.8 Hz, 1H), 5.42 (s, 2H), 5.03 (d, J=13.6 Hz, 1H), 4.97 (s, 2H), 4.51 (t, J=10.0 Hz, 1H), 4.41-4.35 (m, 1H), 4.24-4.16 (m, 3H), 3.63-3.57 (m, 4H), 3.48-3.41 (m, 14H), 3.29-3.27 (m, 1H), 3.09-2.91 (m, 7H), 2.62-2.56 (m, 1H), 2.50-2.44 (m, 1H), 2.40-2.32 (m, 2H), 2.28-2.20 (m, 4H), 2.11 (s, 3H), 2.06-1.88 (m, 5H), 1.80-1.55 (m, 8H), 1.44-1.39 (m, 5H), 1.29-1.24 (m, 11H), 1.12-1.05 (m, 1H), 0.93 (d, J=6.4 Hz, 3H), 0.88-0.78 (m, 17H) ppm.

### (1R,3R)-1-(4-{[(2S)-4-({4-[(2S)-2-[(2S)-2-Amino-3-methylbutanamido]-5-(carbamoylamino)pentanamido]benzenesulfonyl}carbamoyl)-1-(4-fluorophenyl)-4,4-dimethylbutan-2-yl]carbamoyl}-1,3-thiazol-2-yl)-3-[(2S,3S)—N-hexyl-3-methyl-2-{[(2R)-1-methylpiperidin-2-yl]formamido}pentanamido]-4-methylpentyl Acetate (L7-1c)

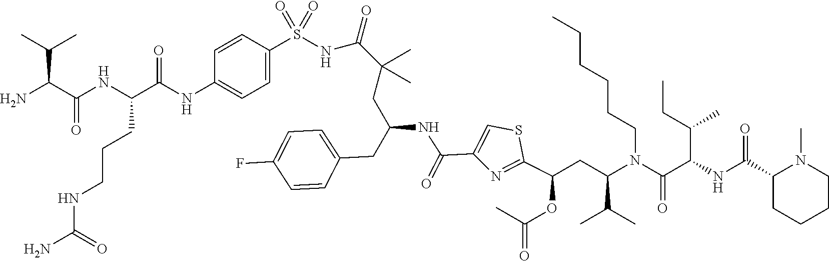 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)NS(=O)(=O)c2ccc(NC(=O)[C@H](CCCNC(N)=O)NC(=O)[C@@H](N)C(C)C)cc2)cs1)C(C)C]

Following a similar procedure for L7-1a except using P47 (40 mg, 41 μmol) instead of P43, compound L7-1c (2.1 mg, 4.2% yield from P47) was obtained as a white solid. ESI m/z: 621 (M/2+H)⁺.

### LP24: (1R,3R)-1-(4-{[(2S)-4-({4-[(2S)-2-[(2S)-2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]benzenesulfonyl}carbamoyl)-1-(4-fluorophenyl)-4,4-dimethylbutan-2-yl]carbamoyl}-1,3-thiazol-2-yl)-3-[(2S,3S)—N-hexyl-3-methyl-2-{[(2R)-1-methylpiperidin-2-yl]formamido}pentanamido]-4-methylpentyl Acetate (LP24)

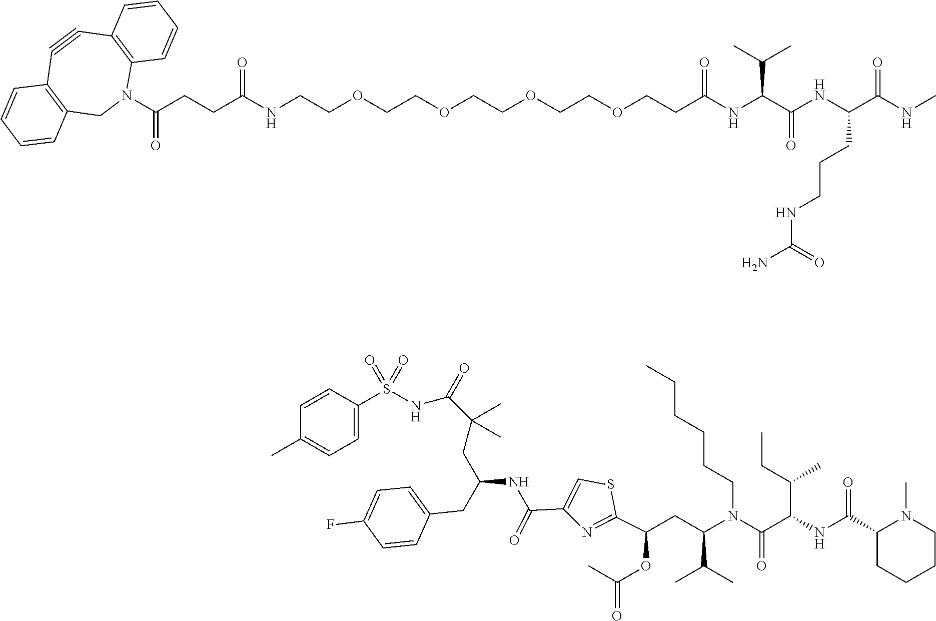

Following General Procedure IX using amine L7-1c (2.1 mg, 1.7 μmol) with OSu ester L0-1c, linker-payload LP24 (1.2 mg, 40% yield) was obtained as a white solid. ESI: 888 (M/2+H)⁺. ¹H NMR (400 MHz, DMSO_{d6}) δ 10.11 (s, 1H), 8.18 (s, 1H), 8.15-8.13 (m, 1H), 7.87-7.83 (m, 2H), 7.76-7.73 (m, 2H), 7.69-7.66 (m, 3H), 7.63-7.61 (m, 2H), 7.56 (s, 1H), 7.51-7.45 (m, 4H), 7.39-7.34 (m, 2H), 7.32-7.28 (m, 1H), 7.18-7.10 (m, 2H), 7.02-6.97 (m, 2H), 5.99-5.98 (m, 1H), 5.63-5.59 (m, 1H), 5.41 (m, 2H), 5.34-5.31 (m, 1H), 5.05-5.01 (m, 1H), 4.51-4.46 (m, 1H), 4.41-4.37 (m, 2H), 4.26-4.23 (m, 2H), 4.11-4.06 (m, 2H), 3.62 (m, 1H), 3.61-3.59 (m, 3H), 3.47 (m, 13H), 3.09-3.07 (m, 1H), 3.02-2.94 (m, 2H), 2.72-2.66 (m, 2H), 2.40-2.37 (m, 2H), 2.35-2.31 (m, 2H), 2.27-2.20 (m, 4H), 2.10 (s, 4H), 2.03-1.95 (m, 8H), 1.89-1.84 (m, 2H), 1.80-1.71 (m, 3H), 1.48-1.44 (m, 4H), 1.24 (m, 3H), 0.96-0.85 (m, 12H), 0.84-0.81 (m, 21H), 0.70-0.68 (m, 4H) ppm.

### (1R,3R)-1-(4-{[(2S)-4-{[4-({[({4-[(2S)-2-[(2S)-2-Amino-3-methylbutanamido]-5-(carbamoylamino)pentanamido]phenyl}methoxy)carbonyl]amino}methyl)benzenesulfonyl]carbamoyl}-1-(4-fluorophenyl)-4,4-dimethylbutan-2-yl]carbamoyl}-1,3-thiazol-2-yl)-3-[(2S,3S)—N-hexyl-3-methyl-2-{[(2R)-1-methylpiperidin-2-yl]formamido}pentanamido]-4-methylpentyl Acetate (L7-1d)

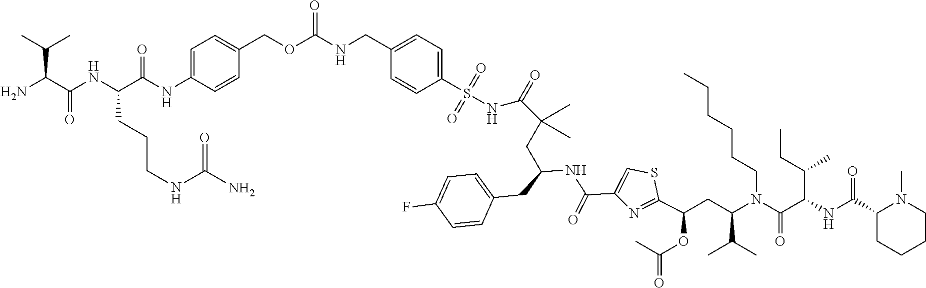 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)NS(=O)(=O)c2ccc(CNC(=O)OCc3ccc(NC(=O)[C@H](CCCNC(N)=O)NC(=O)[C@@H](N)C(C)C)cc3)cc2)cs1)C(C)C]

Following General Procedure X using Fmoc-vcPAB-PNP (L1-1a) with amine P46 (65 mg, 65 μmol), compound Fmoc-L7-1d (76 mg, ESI m/z: 813 (M/2+H)⁺) was obtained as a white solid. Fmoc-L7-1d was dissolved in DMF (5 mL). To the solution was added piperidine (0.4 mL). The reaction mixture was stirred at room temperature for half an hour, and monitored by LCMS. The reaction mixture was purified directly by reversed phase flash chromatography (0-100% acetonitrile in water) to give L7-1d (50 mg contaminated with 5% of P46, 54% yield from P46) as a white solid. ESI m/z: 702 (M+H)⁺.

### LP25: (1R,3R)-1-(4-{[(2S)-4-{[4-({[({4-[(2S)-2-[(2S)-2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methoxy)carbonyl]amino}methyl)benzenesulfonyl]carbamoyl}-1-(4-fluorophenyl)-4,4-dimethylbutan-2-yl]carbamoyl}-1,3-thiazol-2-yl)-3-[(2S,3S)—N-hexyl-3-methyl-2-{[(2R)-1-methylpiperidin-2-yl]formamido}pentanamido]-4-methylpentyl Acetate (LP25)

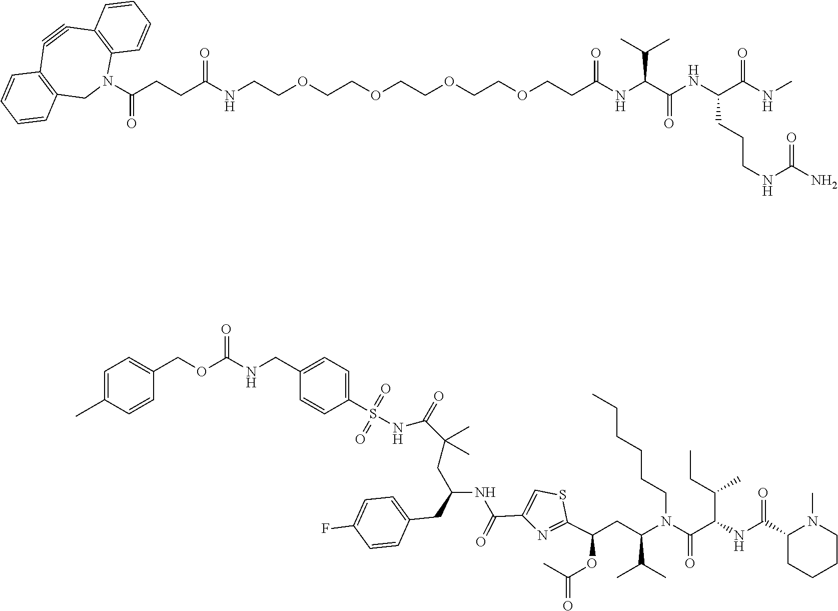 [SMILES: CNC(=O)[C@H](CCCNC(N)=O)NC(=O)[C@@H](NC(=O)CCOCCOCCOCCOCCNC(=O)CCC(=O)N1Cc2ccccc2C#Cc2ccccc21)C(C)C ; CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)NS(=O)(=O)c2ccc(CNC(=O)OCc3ccc(C)cc3)cc2)cs1)C(C)C]

Following General Procedure IX using amine L7-1d (40 mg, 29 μmol) with OSu ester L0-1d, linker-payload LP25 (23 mg, 48% yield) was obtained as a white solid. ESI m/z=647 (M/3+H)⁺. ¹H NMR (400 MHz, DMSO_{d6}) δ 10.02 (s, 1H), 8.18 (s, 1H), 8.15 (d, J=8.0 Hz, 1H), 7.91-7.78 (m, 4H), 7.69-7.59 (m, 6H), 7.51-7.43 (m, 3H), 7.40-7.29 (m, 5H), 7.22 (br s, 2H), 7.15-7.12 (m, 2H), 7.05-7.00 (m, 2H), 6.01 (t, J=8.0 Hz, 1H), 5.60 (d, J=12.0 Hz, 1H), 5.44 (s, 2H), 5.02 (t, J=12.0 Hz, 1H), 4.97 (s, 2H), 4.50 (t, J=12.0 Hz, 1H), 4.38 (d, J=4.0 Hz, 1H), 4.25-4.18 (m, 3H), 4.10-4.07 (m, 1H), 3.63-3.56 (m, 4H), 3.49-3.45 (m, 14H), 3.31-3.28 (m, 1H), 3.09-2.91 (m, 7H), 2.72-2.71 (m, 2H), 2.62-2.54 (m, 2H), 2.40-2.20 (m, 6H), 2.11 (s, 3H), 2.03-1.91 (m, 6H), 1.79-1.65 (m, 7H), 1.57-1.35 (m, 8H), 1.26-1.23 (m, 9H), 1.11-1.08 (m, 1H), 0.97-0.95 (m, 8H), 0.87-0.80 (m, 16H), 0.70 (br s, 3H) ppm. ¹⁹F NMR (376 MHz, DMSO_{d6}) δ −117 ppm.

### LP26-1: (4S)-5-[4-(2-{[({4-[(2S)-2-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-5-methoxy-5-oxopentanamido]-3-methylbutanamido]-5-(carbamoylamino)pentanamido]phenyl}methoxy)carbonyl]amino}acetamido)phenyl]-4-({2-[(1R,3R)-1-ethoxy-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic Acid (LP26-1)

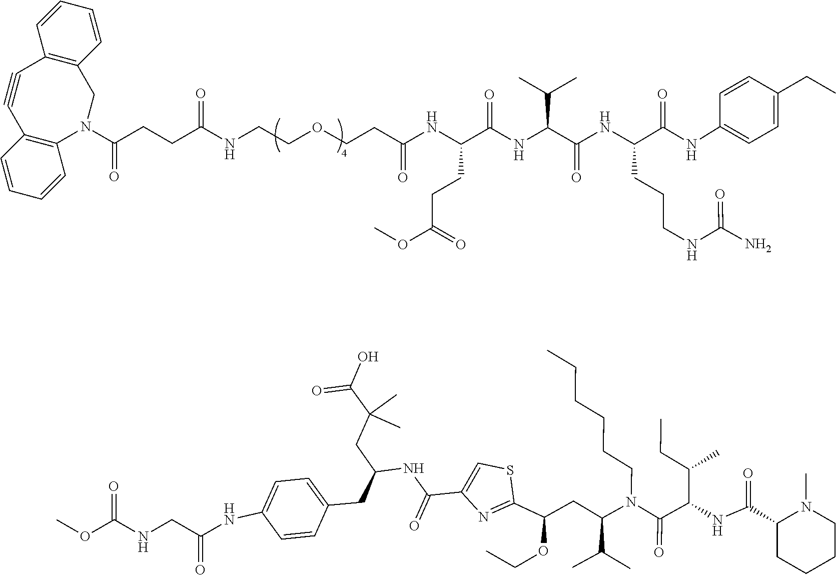

Following the general procedure X starting from P31 (33 mg, 38 μmol) with L5-lb (46 mg, 38 μmol), LP26-1 (55 mg, 74% yield) was obtained as a white solid. ESI m/z: 976.2 (M/2+H)⁺.

### LP26: (4S)-5-[4-(2-{[({4-[(2S)-2-[(2S)-2-[(2S)-2-[1-(4-{2-(4S)-5-[4-(2-{[({4-[(2S)-2-[(2S)-2-[(2S)-2-[1-(4-{2-Azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-4-carboxybutanamido]-3-methylbutanamido]-5-(carbamoylamino)pentanamido]phenyl}methoxy)carbonyl]amino}acetamido)phenyl]-4-({2-[(1R,3R)-1-ethoxy-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic Acid (LP26)

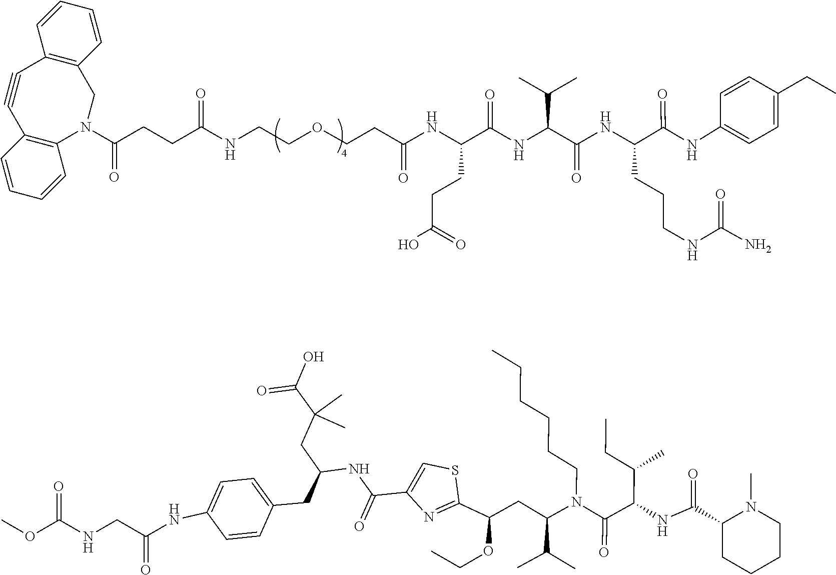

To a solution of LP26-1 (40 mg, 0.02 mmol) in methanol (2 mL) was added aq. lithium hydroxide (2 mL, 0.04 M), and the reaction mixture was stirred at room temperature for 4 hours, which was monitored by LCMS. The reaction mixture was directly purified by reversed phase flash chromatography (0-100% acetonitrile in aq. ammonium bicarbonate (0.05%)) to give LP104 (5 mg, 14% yield) as a white solid. ESI m/z: 647.2 (M/3+H)⁺. ¹H NMR (400 MHz, DMSO_{d6}) δ 10.03 (s, 1H), 9.88 (s, 1H), 8.20-8.13 (m, 2H), 8.07 (d, J=7.8 Hz, 1H), 7.78-7.70 (m, 2H), 7.69-7.65 (m, 1H), 7.65-7.56 (m, 3H), 7.53-7.42 (m, 5H), 7.40-7.27 (m, 4H), 7.09 (d, J=8.4 Hz, 2H), 6.52 (s, 1H), 6.02-5.95 (m, 1H), 5.42 (s, 2H), 5.09-4.93 (m, 4H), 4.56-4.47 (m, 2H), 4.42-4.16 (m, 7H), 3.73-3.80 (m, 4H), 3.65-3.54 (m, 5H), 3.52-3.42 (m, 12H), 3.12-2.84 (m, 8H), 2.80-2.64 (m, 5H), 2.43-2.30 (m, 3H), 2.28-2.19 (m, 3H), 2.17-2.06 (m, 3H), 2.05-1.77 (m, 10H), 1.75-1.53 (m, 8H), 1.51-1.36 (m, 5H), 1.35-1.22 (m, 7H), 1.20-1.13 (m, 3H), 1.07-1.00 (m, 5H), 0.93-0.77 (m, 18H), 0.70 (s, 3H) ppm. (2 active protons were not revealed.)

### ADC Conjugation

### General Procedure for Conjugation

This example demonstrates a method for conjugation of a maleimide-spacer-payload to inter-chain cysteines of an antibody or antigen-binding fragment via the formation of a thioether bond.

Conjugation through antibody cysteines can be performed in two steps using methods similar to those for making Adcetris®-like ADCs (See, Mol. Pharm. 2015, 12(6), 1863-71). A monoclonal antibody (mAb) (10 mg/mL in 50 mM HEPES, 150 mM NaCl) at pH 7-8 can be reduced with 1 mM dithiothreitol (6 molar equiv. of antibody) or TCEP (2.5 molar equivalents to antibody) at 37° C. for 30 min. After gel filtration (G-25, pH 6.3, sodium acetate), a linker-payload at 1-10 mg/mL in DMSO can be added to the reduced antibody, and the reaction is allowed to stir for 3-14 h at rt. The resulting mixture can be purified by SEC to generate pure ADC.

### General Procedure for Site-specific Conjugation

This example demonstrates a method for site-specific conjugation of a cyclooctyne-linker-payload to an antibody or antigen-binding fragment thereof.

In this example, the site-specific conjugates can be produced in two steps. The first step is microbial transglutaminase (MTG) based enzymatic attachment of a small molecule, such as an azido-PEG₃-amine, to the antibody having N297Q mutation (hereinafter “MTG-based” conjugation). The second step uses the attachment of a cyclooctyne-spacer-payload to the azido-functionalized antibody via a [2+3] cycloaddition, for example, the 1,3-dipolar cycloaddition between an azide and a cyclooctyne (aka copper-free click chemistry). See, Baskin, J. M.; Prescher, J. A.; Laughlin, S. T.; Agard, N. J.; Chang, P. V.; Miller, I. A.; Lo, A.; Codelli, J. A.; Bertozzi, C. R. PNAS 2007, 104 (43), 16793-7. This process provided site-specific and stoichiometric conjugates in about 50-80% isolated yield.

### Step 1: Preparation of an Azido-Functionalized Antibody.

Aglycosylated human antibody IgG (IgG1, IgG4, etc.) or a human IgG1 isotype with a N297Q mutation, in PBS (pH 6.5-8.0) is mixed with >200 molar equivalents of azido-PEG₃-amine (ZP3A, MW=218.26 g/mol). The resulting solution is mixed with MTG (EC 2.3.2.13 from Zedira, Darmstadt, Germany, or ACTIVA TI which contains Maltodextrin from Ajinomoto, Japan) (25 U/mL; 5U MTG per mg of antibody) resulting in a final concentration of 0.5-5 mg/mL antibody, and the solution is then incubated at 37° C. for 4-24 h while gently shaking. The reaction can be monitored by ESI-MS. Upon reaction completion, the excess amine and MTG can be removed by SEC or protein A column chromatography, to generate the azido-functionalized antibody. This product can be characterized by SDS-PAGE.

In certain experiments, the N297Q antibody (24 mg) in 7 mL potassium-free PBS buffer (pH 7.3) is incubated with >200 molar equivalents of the azido-PEG₃-amine ZP3A (MW=218.26) in the presence of MTG (0.350 mL, 35 U, mTGase, Zedira, Darmstadt, Germany). The reaction is incubated at 37° C. overnight while gently mixing. Excess azido-PEG₃-amine and mTGase can be removed by size exclusion chromatography (SEC, Superdex 200 PG, GE Healthcare).

Step 2: Preparation of site-specific conjugates by a [2+3] click reaction between the azido-functionalized transglutaminase-modified antibodies (IgG1, IgG4, etc.) and cyclooctyne containing linker-payloads (LPs). In general, an azido-functionalized aglycosylated antibody-LP conjugate can be prepared by incubating the azido-functionalized transglutaminase-modified antibody (1 mg) in 1 mL of an aqueous medium (e.g., PBS, PBS containing 5% glycerol, HBS) with >6 molar equivalents of an LP dissolved in a suitable organic solvent (e.g., DMSO, DMF or DMA; reaction mixture contains 10-20% organic solvent, v/v) at 24° C. to 32° C. for over 3 hours. The progress of the reaction can be monitored by ESI-MS. Absence of azido-functionalized or transglutaminase-modified antibody (mAb-PEG₃-N₃) indicated completion of the conjugation. The excess linker-payload (LP) and organic solvent can be removed by SEC (Waters, Superdex 200 Increase, 1.0×30 cm, GE Healthcare, flow rate 0.8 mg/mL, PBS, pH 7.2) eluting with PBS, or via protein A column chromatography via elution with acidic buffer followed by neutralization with Tris (pH 8.0). The purified conjugate can be analyzed by SEC, SDS-PAGE, and ESI-MS.

In certain examples, the azido-functionalized antibody (1 mg) in 0.800 mL PBSg (PBS, 5% glycerol, pH 7.4) can be treated with six equivalents of DIBAC-Suc-PEG₄-VC-PABC-payload (conc. 10 mg/mL in DMSO) for 6 hours at rt and the excess linker payload (LP) can be removed by size exclusion chromatography (SEC, Superdex 200 HR, GE Healthcare). The final product can be concentrated by ultra-centrifugation and characterized by UV, SEC, SDS-PAGE and/or ESI-MS.

### Preparation of ADCs 1-37

Step 1: In this step, the antibody is site-specifically functionalized at glutamine residues with an azido-alkyl amine. Specifically, anti-Her2 human IgG antibody containing an N297Q mutation (TRSQ) or isotype control antibody containing the same mutation (CTRL) was mixed with excess, e.g., 20-100 molar equivalents of the appropriate azido-alkyl amine. The resulting solution was mixed with transglutaminase (1U mTG per mg of antibody, Millipore-Sigma) resulting in a final concentration of the antibody at 1-20 mg/mL. The reaction mixture was incubated at 25-37° C. for 4-24 hours while gently shaking. Reaction progress was monitored by ESI-MS. Upon completion, excess amine and mTG were removed by size exclusion chromatography (SEC) or protein A column chromatography. The conjugate was characterized by UV-Vis, SEC and ESI-MS.

Step 2: In this step, the antibody produced in Step 1 is conjugated with a linker payload via cyloaddition reaction. Specifically, the azido-functionalized antibody from Step 1 was incubated (1-20 mg/mL) in PBS (pH7.4) with 10-20 molar equivalents of a linker-payload dissolved in an organic solvent (e.g., DMSO or DMA (10 mg/mL)) to obtain a reaction mixture that is approximately 5-15% organic solvent (v/v), at 25-37° C. for 1-48 hours while gently shaking. The reaction was monitored by ESI-MS. Upon completion, the excess amount of linker-payload and protein aggregates were removed by size exclusion chromatography (SEC). The purified conjugate was concentrated, sterile filtered and characterized by UV-Vis, SEC and ESI-MS. Conjugates monomer purity was >99% by SEC. General Procedure for Characterization of Antibody and ADCs

The purified conjugates can be analyzed by SEC, ESI-MS, and SDS-PAGE. Characterization of ADC by SEC

Analytical SEC experiments can be run using a Waters 1515 instrument, on a Superdex™ 200 Increase (1.0×30 cm) column, at flow rate of 0.80 mL/min using PBS pH 7.2, and monitored at X=280 nm using a Waters 2998 PDA. An analytic sample is composed of 200 μL PBS (pH 7.4) with 30-100 μL of test sample. Preparative SEC purifications can be performed using an AKTA Avant instrument from GE Healthcare, on Superdex 200 PG (2.6×60 cm) column, at a flow rate 2 mL/min eluting with PBS pH 7.2, and monitored at X=280 nm. The SEC results typically indicate retention times for monomeric mAb and conjugates thereof, with minimal aggregation or degradation.

### Characterization of ADC by LC-ESI-MS

Measurement of intact mass for the ADC samples by LC-ESI-MS can be performed to determine drug-payload distribution profiles and to calculate the average DAR. Each testing sample (20-50 ng, 5 μL) is loaded onto an Acquity UPLC Protein BEH C₄ column (10K psi, 300 Å, 1.7 μm, 75 m×100 mm; Cat No. 186003810). After desalting for 3 min, the protein can be eluted and mass spectra can be acquired by a Waters Synapt G2-Si mass spectrometer. Most site-specific ADCs have near 4DAR.

### Characterization of ADC by SDS-PAGE

SDS-PAGE can be used to analyze the integrity and purity of the ADCs. In one method, SDS-PAGE conditions include non-reduced and reduced samples (2-4 μg) along with BenchMark Pre-Stained Protein Ladder (Invitrogen, cat #10748-010; L #1671922.) loaded per lane in (1.0 mm×10 well) Novex 4-20% Tris-Glycine Gel and can be ran at 180 V, 300 mA, for 80 min. An analytical sample is prepared using Novex Tris-Glycine SDS buffer (2×) (Invitrogen, cat # LC2676) and the reduced sample is prepared with SDS sample buffer (2×) containing 10% 2-mercaptoethanol.

### In Vitro Plasma Stability

To determine the plasma stability of representative ADCs containing the tubulysin payloads or prodrug payloads, ADCs can be incubated in vitro with plasma from different species, and the DAR is evaluated after incubation at physiological temperature (37° C.) for 3 days.

For the assay, each ADC sample in PBS buffer is added to fresh pooled male mouse, cynomologus monkey, rat, or human plasma, separately, at a final concentration of 50 μg/mL in a 96-well plate, and subsequently incubated at 37° C. for 72 hours. After incubation, each sample (100 μL final volume) is individually frozen at −80° C. until analysis.

Affinity capture of the ADCs from the plasma samples can be carried out on a KingFisher 96 magnetic particle processor (Thermo Electron). First, biotinylated extracellular domain of human PRLR expressed with a myc-myc hexahistidine tag (hPRLR ecto-MMH 100 g/mL) is immobilized on streptavidin paramagnetic beads (In vitrogen, Cat #60210). Each plasma sample containing tubulysin ADCs (100 μL) is mixed at 600 rpm with 100 μL of the beads (the commercial beads come in volume) at room temperature for 2 hours in a 96-well plate. The beads are then washed three times with 600 μL of HBS-EP (GE healthcare, Cat #BR100188), once with 600 μL of H₂O, and then once with 600 μL of 10% acetonitrile in water. Following the washes, tubulysin ADCs can be eluted by incubating the beads with 70 L of 1% formic acid in 30% acetonitrile/70% water for 15 minutes at room temperature. Each eluate sample is then transferred into a v-bottom 96-well plate and is then reduced with 5 mM TCEP (Thermo Fisher, Cat #77720) at room temperature for 20 minutes.

The reduced tubulysin ADC samples (10 μL/sample) can be injected onto a 1.7 μm BEH300 C₄ column (Waters Corporation, Cat #186005589) coupled to a Waters Synapt G2-Si Mass Spectrometer. The flow rate is 0.1 mL/min (mobile phase A: 0.1% formic acid in water; mobile phase B: 0.1% formic acid in acetonitrile). The LC gradient starts with 20% B and increases to 35% B in 16 minutes, then reaches 95% B in 1 minute.

The acquired spectra can be deconvoluted using MaxEnt1 software (Waters Corporation) with the following parameters: Mass range: 20-30 kDa for the light chain, and 40-60 kDa for the heavy chain; m/z range: 700 Da-3000 Da; Resolution: 1.0 Da/channel; Width at half height: 1.0 Da; Minimum intensity ratios: 33%; Iteration max: 25.

Significant loss of linker-payloads is typically not observed from the tested ADCs after 72-hour incubation with human, mouse, rat, and cynomolgus monkey plasma. However, the acetyl group of tubulysin payloads or prodrug payloads can be hydrolyzed to a hydroxyl group (−43 Da) with significant loss of toxicity. Therefore, the hydrolyzed species observed in the LC-MS is considered as loss of drug. Drug/antibody ratio (DAR) can be calculated based on the relative abundance of different species of heavy chains.

$$\begin{matrix}Drug / antibody Ratio ( D A R ) \\ 2 \times \end{matrix} = \frac{2 \times Intensity ( heavy chain with 2 drugs ) + 1 \times Intensity ( heavy chain with 1 drug )}{Sum Intensity ( Heavy chain with 2 , 1 and 0 drugs )}$$

### Testing of Tubulysin Payloads in Cell-Based Killing Assays

To test the ability of the disclosed tubulysin payloads or prodrug payloads to kill human cell lines, an in vitro cytotoxicity assay can be performed. In vitro cytotoxicity of the disclosed payloads, as well as reference compounds, are evaluated using the CellTiter-Glo Assay Kit (Promega, Cat # G7573), in which the quantity of ATP present is used to determine the number of viable cells in culture. For the assay, C4-2, HEK293, or T47D cells are seeded at 4000 cells/well on Nunclon white 96-well plates in complete growth medium (DME high glucose:Ham's F12 at 4:1, 10% FBS, 100 units/ml Penicillin, 100 ug/ml streptomycin, 53 ug/ml glutatmine, 10 ug/ml insulin, 220 ng/ml biotin, 12.5 pg/ml T3, 12.5 ug/ml Adenine, 4 ug/ml transferrin for C42 cells; DME high glucose, 10% FBS, 100 units/ml Penicillin, 100 ug/ml streptomycin, 53 ug/ml glutatmine for HEK293; RPMI, 10% FBS, 100 units/ml Penicillin, 100 ug/ml streptomycin, 53 ug/ml glutatmine, 10 ug/ml insulin, 10 mM HEPEs, 200 nM Sodium Pyruvate for T47D cells) and grown overnight at 37° C. in 5% CO₂. For cell viability curves, 1:3 serially diluted payloads are added to the cells at final concentrations ranging from 100 nM to 15 pM, including a no treatment control group, and are then incubated for 5 days. After the 5-day incubation, cells are incubated at room temperature with 100 μL of CellTiter-Glo reagents for 10 minutes. Relative luminescence units (RLU) can be determined on a Victor plate reader (PerkinElmer). The IC₅₀ values are determined from a four-parameter logistic equation over a 10-point response curve (GraphPad Prism). All IC₅₀ values are expressed in molar (M) concentration. The percent cell killing (% kill) at the maximum concentration tested is estimated from the following formula (100−% viable cells). Averages±standard deviation (SD) can be included where replicate experiments are performed.

Payloads and prodrug payloads herein can demonstrate killing of C4-2 cells with IC₅₀ values between 16 pM and >100 nM, and maximum % cell killing between 8.9% and 96.7%. A subset of disclosed payloads can demonstrate killing of HEK293 cells with IC₅₀ values between 57 pM and >100 nM, and maximum % cell killing between 4% and 89%. A subset of disclosed payloads can demonstrate killing of T47D cells with IC₅₀ values between 35 pM and >100 nM, and maximum % cell killing between 15% and 85%. The reference compound, MMAE, demonstrates killing of C4-2 cells with IC₅₀ values of 283 pM, and a maximum % cell killing of 93.7%.

### Testing of Tubulysin Payloads in MDR Cell Based Killing Assays

To further test the ability of the disclosed tubulysin payloads, a cytotoxicity assay can be performed using a multidrug resistant (MDR) cell line with or without Verapamil, a drug that has been shown to reverse drug resistance (Cancer Res. 1989 Sep. 15; 49(18):5002-6). In vitro cytotoxicity of the disclosed payloads as well as reference compounds are evaluated similarly as described above except using 1000 HCT15 cells, a colorectal carcinoma cell line, in growth medium (RPMI, 10% FBS, 100 units/ml Penicillin, 100 ug/ml streptomycin, 53 ug/ml glutatmine) with or without 5 ug/mL of Verapamil.

In the absence of Verapamil, payloads of the disclosure can demonstrate killing of HCT15 cells with IC₅₀ values between 20 pM and >100 nM, and maximum % cell killing between −3.8 and 99.7%. In the presence of Verapamil, payloads of the disclosure can demonstrate killing of HCT15 cells with IC₅₀ values between 15 pM and >100 nM, and maximum % cell killing between −0.4% and 99.1%. For each payload or prodrug payload, the HCT-15 IC₅₀ in the absence of Verapamil is divided by the HCT-15 IC₅₀ in the presence of Verapamil (HCT-15 IC₅₀/HCT-15+Verapamil IC₅₀). Several payloads can have ratios <2.0 suggesting that these payloads are minimally impacted by multi-drug efflux pumps. The reference compound, (MMAE), can have a ratio of 23.7.

### Testing of Tubulysin Payloads in a Panel of MDR Cell Lines

To further test the ability of the disclosed tubulysin payloads, a cytotoxicity assay can be performed using a panel of multidrug resistant (MDR) cell lines. In vitro cytotoxicity of the disclosed payloads as well as reference compounds are similarly evaluated as described above except using HCT-15 cells, a colorectal carcinoma cell line; H69AR, a doxorubicin resistant MDR derivative of the small cell lung cancer carcinoma cell line NCI-H69; MES-SA/MX2, a mitoxantrone resistant MDR derivative of the uterine sarcoma cell line MES-SA; and HL60/MX2, a mitoxantrone resistant MDR derivative of the acute promyelocytic leukemia cell line HL60. In these assays, cytotoxicity is evaluated in normal growth media (RPMI, 10% FBS, 100 units/ml Penicillin, 100 ug/ml streptomycin, and 53 ug/ml glutatmine for HCT-15 and HL60/MX2; RPMI, 20% FBS, 100 units/ml Penicillin, 100 ug/ml streptomycin, and 53 ug/ml glutatmine for H69-AR; Waymouths's:McCoy's (1:1), 10% FBS, 100 units/ml Penicillin, 100 ug/ml streptomycin, and 53 ug/ml glutatmine for MES-SA/MX2) with 1000 cells per well following 72 h and 144 h incubation with payloads. Some payloads can kill the entire panel of MDR cell lines with sub nM IC₅₀, and to near baseline levels suggesting that these payloads can overcome MDR in the tested lines.

Testing of tubulysin payload containing ADCs in cell based killing assays

Bioassays can be developed to assess the efficacy of an anti-PRLR antibody conjugated with the disclosed tubulysin payloads or prodrug payloads and reference payloads. to the assays can assess the activity of tubulysin payloads after internalization of an anti-PRLR-tubulysin ADC into cells, release of the payload, and subsequent cytotoxicity. For this assay, a HCT15 line can be engineered to express human full length PRLR (accession #NP_000940.1). The resulting stable cell line is referred to herein as HCT15/PRLR. In vitro cytotoxicity of the disclosed payloads, reference compounds, and tested ADCs are evaluated similarly, as described in this example, using HCT15/PRLR cells with or without 5 pg/mL of Verapamil diluted in normal culture medium. The compounds are tested at concentrations starting at 100 nM with 3-fold serial dilution. All IC₅₀ values are expressed in nM concentration and the percent cell killing (% kill) at the maximum concentration tested was estimated from the following formula (100−% viable cells).

In the absence of Verapamil, anti-PRLR ADCs conjugated with disclosed linker-payloads, can demonstrate cytotoxicity in a HCT15/PRLR cell based assay at an IC₅₀ value of 0.5 nM, with maximum percent killing of 90%; and at an IC₅₀ value of 3 nM, with maximum percent killing of 65%, respectively. Under these conditions, one isotype control ADC demonstrated some modest killing of HCT15/PRLR cells with a maximum percent killing of 51%, but the IC₅₀ value was >50 nM. In the absence of Verapamil, another isotype control did not demonstrate any significant killing of HCT15/PRLR cells. Under these conditions, the free payloads of this disclosure, can demonstrate killing of HCT15/PRLR cells with IC₅₀ values of 0.04 nM and 0.2 nM, and maximum percent killing of 99% and 99%, respectively.

In the presence of Verapamil, anti-PRLR ADCs conjugated with linker-payloads or linker-prodrug payloads of this disclosure, can demonstrate cytotoxicity in HCT15/PRLR cell-based assay at an IC₅₀ value of 0.3 nM, with maximum percent killing of 91%; and at an IC₅₀ value of 0.2 nM, with maximum percent killing of 91%, respectively. Under these conditions, two Isotype control ADCs can demonstrate killing of HCT15/PRLR cells with an IC₅₀ value greater than 50 nM, and a maximum percent killing of 82%; and an IC₅₀ value greater than 50 nM, and a maximum percent killing of 76%, respectively. Under these conditions, the disclosed free payload scan demonstrate killing of HCT15/PRLR cells with IC₅₀ values of 0.015 nM and 0.033 nM, and maximum percent killing of 99% and 99%, respectively. The unconjugated anti-PRLR antibody did not demonstrate any killing of HCT15/PRLR cells in the presence or absence of Verapamil.

To further test the ability of the disclosed tubulysin payloads, reference compounds, and antibody drug conjugates using these payloads, a cytotoxicity assay can be performed using C4-2 cells as described in this example. For these studies, anti-STEAP2 antibodies were conjugated to select tubulysins payloads, and the compounds can be tested at concentrations starting at 100 nM with 3-fold serial dilution. All IC₅₀ values are expressed in nM concentration and the percent cell killing at the maximum concentration tested is estimated from the following formula (100−% viable cells).

Anti-STEAP2 ADCs conjugated with disclosed linker-payloads can demonstrate cytotoxicity in the C4-2 cell based assay at an IC₅₀ value of 0.1 nM, with maximum percent killing of 99%; an IC₅₀ value of 0.15 nM, with maximum percent killing of 99%; and an IC₅₀ of 0.28 nM with maximum percent killing of 96%, respectively. The reference ADC, anti-STEAP2-MMAE can demonstrate cytotoxicity in the C4-2 cell-based assay with an IC₅₀ value of 0.53 nM, with maximum percent killing of 99%. All three isotype controls can demonstrate some modest killing of C4-2 cells at only the highest tested concentrations with a maximum percent killing of 16%-48%, but an IC₅₀ value >100 nM. Free reference payload MMAE can demonstrate killing of C4-2 cells with IC₅₀ value of 0.22 nM, and maximum percent killing of 99%. The unconjugated anti-STEAP2 antibody did not demonstrate any killing of C4-2 cells.

### Anti-STEAP2 Antibodies

To determine the in vivo efficacy of anti-STEAP2 antibodies conjugated to tubulysins, studies can be performed in immunocompromised mice bearing STEAP2 positive C4-2 prostate cancer xenografts.

For the assay, 7.5×10⁶ C4-2 cells (ATCC, Cat # CRL-3314), which endogenously express STEAP2, are suspended in Matrigel (BD Biosciences, Cat #354234) and implanted subcutaneously into the left flank of male CB17 SCID mice (Taconic, Hudson N.Y.). Once tumors reach an average volume of 220 mm³ (around Day 15), mice are randomized into groups of seven and given a single dose of either anti-STEAP2 conjugated antibodies, isotype control conjugated antibody, or vehicle at 2.5 mg/kg via tail vein injection. Tumors are measured with calipers twice a week until the average size of the vehicle group reached 1500 mm³. Tumor size is calculated using the formula (length×width²)/2 and the average tumor size+/−SEM is then calculated. Tumor growth inhibition is calculated according to the following formula: (1−((T_{final}−Tᵢₙᵢₜᵢₐₗ)/(C_{final}−Cᵢₙᵢₜᵢₐₗ)))*100, where treated group (T) and control group (C) represent the mean tumor mass on the day the vehicle group reaches 1500 mm³.

In this study, anti-STEAP2 antibody conjugated to MMAE is compared to anti-STEAP2 antibody conjugated to tubulysin linker-payloads for their ability to reduce C4-2 tumor size. Treatment with anti-STEAP2-MMAE reference ADC typically results in an average of 81% tumor growth inhibition at the completion of the study. Treatment with the isotype control ADCs typically leads to an average of 31-33% reduction in tumor growth. The anti-STEAP2 antibodies comprise N297Q mutations.

### Efficacy of STEAP2-Tubulysin ADC in CTG-2440 and CTG-2441 PDX Prostate Cancer Models

### Experimental Procedure:

Prostate cancer Patient-Derived Xenograft (PDX) tumor fragments of either CTG-2440 or CTG-2441 can be implanted subcutaneously into the flank of male NOG mice. Once the tumor volumes reach approximately 200 mm³, mice are randomized into groups of eight and are treated. Tumor growth is monitored for 60 days post-implantation.

### Results and Conclusions:

The anti-tumor efficacy of a STEAP2 Tubulysin ADC in a STEAP2 positive PDX model is assessed relative to control ADC. CTG-2440 tumors treated with the control ADC can grow to protocol size limits within 28 days. Growth of tumors treated with STEAP2 Tubulysin ADC can be inhibited for 60 days with no deleterious effect on body weight change. The anti-tumor efficacy is dose dependent. Complete tumor inhibition is observed with a total payload dose of 240 ug/kg, while tumor regression is induced with 120 ug/kg and 40 ug/kg total payload doses.

CTG-2441 tumors treated with the control ADC can grow to protocol size limits within 30 days. Growth of tumors treated with STEAP2 Tubulysin ADC can be inhibited for 60 days with only moderate weight loss observed. The anti-tumor efficacy is dose dependent. Complete tumor inhibition is observed with a total payload dose of either 120 or 240 ug/kg. Tumor regression is induced following a single administration of 40 ug/kg total payload dose.

### PDX Model and STEAP2 Expression Information

The prostate cancer models are derived from the bone metastases of patients with metastatic castrate resistant prostate cancer (mCRPC). STEAP2 expression is confirmed by RNA sequencing data and RNA in situ hybridization.

### Testing of Tubulysin Payloads in a Panel of SK-BR-3 Cell Lines

Anti-proliferation assays were performed using a SK-BR-3 human breast adenocarcinoma (pleural effusion) cell line. The cells were grown in McCoy's 5a Medium supplemented with 10% FBS, penicillin/streptomycin and L-glutamine. Cells were seeded 1000/well in 96-well plate in 80ul complete growth media one day prior to adding ADCs and incubated at 37° C. 5% CO₂ overnight.

The ADCs were 1:3 serially diluted 10 points in assay media (Opti-MEM+0.1% BSA). The concentrations of the testing ADCs cover the range of 1 nM to ˜1000 nM and also starting from different concentrations based on the cell killing potency in order to see EC₅₀ covers, leaving the last well (10^{th}) as blank (no ADC or compound). ADCs were first 1:3 serially diluted 10 points in DMSO starting from 5.0 pM (the starting concentration of each ADC is different according to the EC₅₀s), leaving the last well as blank (containing only DMSO). 10 μl DMSO-diluted compound was transferred to 990 μl assay media (Opti-MEM+0.1% BSA) in a 96-well deep well dilution plate. 20 μl assay media-diluted ADC was added to cells. Cells were incubated at 37° C. 500 CO₂ for 6 days (144 hrs). Plates were developed by adding 100 μl CTG reagent/well to the cells CellTiter-Glo®, from Promega, Cat. No G7573), shaken at room temperature for 10 min, sealed with white adhesive bottom seal and luminescence was read with Envision. Cell kill %=[1−(T144ₛₐₘₚₗₑ−T144_{blank})/(T144_{DMSO}−T144_{blank})]×100%, wherein T144 is the data at 144 hours.

The table below provides the drug-antibody ratios (DARs) for conjugates 1-37, along with the EC₅₀ results from the SKBR assays for the same conjugates. The following linker-payloads (from Table P1) were prepared as described in PCT/US2019/068185, the content of which is hereby incorporated by reference in its entirety: LP4-Ve, LP4-Ve, LP25-Ve, LP25-Ve, LP26-Ve, LP26-Ve, LP17-Ve, LP13-Ve, LP19-Ve, LP2-Ve, LP21-Ve, LP6-Vb, LP24-Vb, LP23-Vb, and LP15-VIh.

TABLE 6
ADC Conjugation and SKBR Cell Kill Assay

| | | | ADC | | | |
|---|---|---|---|---|---|---|
| Payload | Linker-payload | | | | | SKBR3 EC₅₀ |
| No. | No. | Name | Name | No. | DAR | (nM) |
| P34 | LP4 | BCN-PEG4-EvcPAB-P34 | TRSQ-ZP3A-LP4 | 1 | 3.89 | 0.023 |
| P34 | LP5 | COT-GGG-P34 | TRSQ-ZP3A-LP5 | 2 | 3.83 | 0.090 |
| P34 | LP6 | BCN-GGGG- P34 (SEQ ID NO: 22) | TRSQ-ZP3A-LP6 | 3 | 3.93 | 0.040 |
| P34 | LP7 | DIBAC-PEG4-GGFG- P34 (SEQ ID NO: | TRSQ-ZP3A-LP7 | 4 | 3.65 | 0.036 |
| | | 23) | | | | |
| P34 | LP8 | BCN-PEG4-GGFG- P34 (SEQ ID NO: 23) | TRSQ-ZP3A-LP8 | 5 | 3.77 | 0.065 |
| P51 | LP9 | COT-PEG3-HOPAS-P51 | TRSQ-ZP3A-LP9 | 6 | 0.71 | 0.220 |
| P1 | LP11 | BCN-PEG4-GGFG-P1 (SEQ ID NO: 23) | TRSQ-ZP3A-LP11 | 7 | 3.68 | 1.083 |
| P1 | LP10 | BCN-GGFG-P1 (SEQ ID NO: 23) | TRSQ-ZP3A-LP10 | 8 | 3.66 | 1.591 |
| P7 | LP12 | DIBAC-PEG4-vcPAB-Gly-P7 | TRSQ-ZP3A-LP12 | 9 | 3.80 | 0.029 |
| P8 | LP13 | DIBAC-PEG4-vcPAB-P8 | TRSQ-ZP3A-LP13 | 10 | 3.86 | 0.083 |
| P8 | LP26 | DIBAC-PEG4-EvcPAB-Gly-P8 | TRSQ-ZP3A-LP26 | 11 | 4.00 | 0.078 |
| P8 | LP26 | DIBAC-PEG4-EvcPAB-Gly-P8 | CTRL-ZP3A-LP26 | 12 | 4.00 | 58.334 |
| P5 | LP16 | DIBAC-PEG4-EvcPAB-P5 | TRSQ-ZP3A-LP16 | 13 | 3.93 | 0.182 |
| P5 | LP18 | DIBAC-PEG4-GGG-P5 | TRSQ-ZP3A-LP18 | 14 | 3.94 | 0.118 |
| P5 | LP19 | BCN-PEG4-GGFG-P5 (SEQ ID NO: 23) | TRSQ-ZP3A-LP19 | 15 | 3.91 | 0.135 |
| P11 | LP20 | DIBAC-PEG4-vcPAB-P11 | TRSQ-ZP3A-LP20 | 16 | 3.95 | 1.366 |
| P11 | LP21 | DIBAC-P11 | TRSQ-ZP3A-LP21 | 17 | 3.83 | 0.232 |
| P43 | LP22 | DIBAC-PEG4-vc-P43 | TRSQ-ZP3A-LP22 | 18 | 3.86 | 0.376 |
| P43 | LP22 | DIBAC-PEG4-vc-P43 | CTRL-ZP3A-LP22 | 19 | 2.18 | 461.721 |
| P46 | LP25 | DIBAC-PEG4-vcPAB-P46 | TRSQ-ZP3A-LP25 | 20 | 3.04 | 1.329 |
| P47 | LP24 | DIBAC-PEG4-vc-P47 | TRSQ-ZP3A-LP24 | 21 | 3.82 | 2.733 |
| P47 | LP24 | DIBAC-PEG4-vc-P47 | CTRL-ZP3A-LP24 | 22 | 4.00 | 321.547 |
| P34 | LP4-Ve | DIBAC-PEG4-vc PAB-P34 | TRSQ-ZP3A- LP4-Ve | 23 | 4.00 | 0.064 |
| P34 | LP4-Ve | DIBAC-PEG4-vc PAB-P34 | CTRL-ZP3A- LP4-Ve | 24 | 4.00 | 23.401 |
| P34 | LP25-Ve | COT-EDA-(GLCA)PAB-P34 | TRSQ-ZP3A- LP25- | 25 | 3.75 | 0.016 |
| | | | Ve | | | |
| P34 | LP25-Ve | COT-EDA-(GLCA)PAB-P34 | CTRL-ZP3A- LP25- | 26 | 4.00 | 80.211 |
| | | | Ve | | | |
| P34 | LP26-Ve | COT-EDA-(GLC)PAB-P34 | TRSQ-ZP3A- LP26- | 27 | 4.00 | 0.087 |
| | | | Ve | | | |
| P34 | LP26-Ve | COT-EDA-(GLC)PAB-P34 | CTRL-ZP3A- LP26- | 28 | 4.00 | 3.282 |
| | | | Ve | | | |
| P34 | LP17-Ve | COT-GG-P34 | TRSQ-ZP3A- LP17- | 29 | 3.95 | 0.098 |
| | | | Ve | | | |
| P34 | LP13-Ve | DIBAC-GGG-P34 | TRSQ-ZP3A- LP13- | 30 | 4.00 | 0.078 |
| | | | Ve | | | |
| P34 | LP19-Ve | COT-GGGG-P34 (SEQ ID NO: 22) | TRSQ-ZP3A- LP19- | 31 | 3.51 | 0.029 |
| | | | Ve | | | |
| P34 | LP20-Ve | DIBAC-GGFG-P34 (SEQ ID NO: 23) | TRSQ-ZP3A- LP20- | 32 | 3.92 | 0.040 |
| | | | Ve | | | |
| P34 | LP21-Ve | COT-GGGLE-P34 (SEQ ID NO: 24) | TRSQ-ZP3A- LP21- | 33 | 1.58 | 0.023 |
| | | | Ve | | | |
| Vb | LP6-Vb | DIBAC-PEG4-vc-Vb | TRSQ-ZP3A- LP6-Vb | 34 | 3.95 | 0.046 |
| Vb | LP24-Vb | DIBAC-GGFG-Vb (SEQ ID NO: 23) | TRSQ-ZP3A- LP24- | | | |
| | | | Vb | 35 | 3.95 | 0.040 |
| Vb | LP23-Vb | COT-GGGG-Vb (SEQ ID NO: 22) | TRSQ-ZP3A- LP23- | | | |
| | | | Vb | 36 | 3.92 | 0.063 |
| Vlh | LP15- | DIBAC-PEG4-vcPAB-Vlh | TRSQ-ZP3A- LP15- | 37 | 3.37 | 0.044 |
| | Vlh | | Vlh | | | |

1. A compound having the following formula 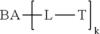
or a pharmaceutically acceptable salt thereof, wherein
BA is a binding agent;
L is a linker covalently bound to BA and to T;
T is 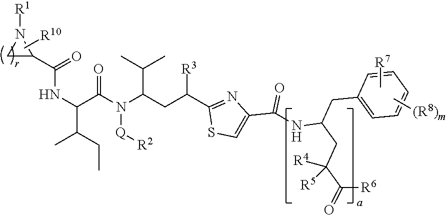
wherein
R¹ is a bond, hydrogen, C₁-C₁₀ alkyl, a first N-terminal amino acid residue, a first amino acid residue, —C₁-C₁₀ alkyl-NR^{3a}R^{3b}, or —C₁-C₁₀ alkyl-OH;
R³ is hydroxyl, —O—, —O—C₁-C₅ alkyl, —OC(O)C₁-C₅ alkyl, —OC(O)N(H)C₁-C₁₀ alkyl, —OC(O)N(H)C₁-C₁₀ alkyl-NR^{3a}R^{3b}, —NHC(O)C₁-C₅ alkyl, or —OC(O)N(H)(CH₂CH₂O)ₙC₁-C₁₀ alkyl-NR^{3a}R^{3b},
wherein R^{3a} and R^{3b} are independently in each instance, a bond, hydrogen, alkyl, alkenyl, alkynyl, cycloalkyl, aryl, heteroaryl, and acyl; wherein alkyl, alkenyl, alkynyl, cycloalkyl, aryl, heteroaryl, and acyl are optionally substituted;
R⁴ and R⁵ are, independently in each instance, hydrogen or C₁-C₅ alkyl;
R⁶ is —OH, —O—, —NHNH₂, —NHNH—, —NHSO₂(CH₂)ₐ₁-aryl-(CH₂)ₐ₂NR^{6a}R^{6b},
wherein aryl is substituted or unsubstituted; and
R^{6a} and R^{6b} are independently in each instance, a bond, hydrogen, alkyl, alkenyl, alkynyl, cycloalkyl, aryl, heteroaryl, and acyl; wherein alkyl, alkenyl, alkynyl, cycloalkyl, aryl, heteroaryl, and acyl are optionally substituted;
R⁷ is, independently in each instance, hydrogen, —OH, —O—, halogen, or —NR^{7a}R^{7b},
wherein R^{7a} and R^{7b} are, independently in each instance, a bond, hydrogen, alkyl, alkenyl, alkynyl, cycloalkyl, aryl, heteroaryl, acyl, —C(O)CH₂OH, —C(O)CH₂O—, a first N-terminal amino acid residue, a first amino acid residue, a first N-terminal peptide residue, a first peptide residue, —CH₂CH₂NH₂, and —CH₂CH₂NH—; wherein alkyl, alkenyl, alkynyl, cycloalkyl, aryl, heteroaryl, and acyl are optionally substituted;
R⁸ is, independently in each instance, hydrogen, —NHR⁹, or halogen,
wherein R⁹ is hydrogen, —C₁-C₅ alkyl, or —C(O)C₁-C₅ alkyl; and
m is one or two;
R¹⁰, when present, is —C₁-C₅ alkyl;
Q is —CH₂— or —O— wherein
R² is alkyl, alkylene, alkynyl, alkynylene, a regioisomeric triazole, a regioisomeric triazolylene;
wherein said regioisomeric triazole or regioisomeric triazolylene is unsubstituted or substituted with alkyl, alkenyl, alkynyl, cycloalkyl, aryl, heteroaryl, or acyl;
wherein n is an integer from one to ten;
wherein r is an integer from one to six;
wherein a, a1, and, a2 are, independently, zero or one; and
k is an integer from one to thirty;
wherein T is not compound IVa, IVa′, IVb, IVc, IVd, IVe, IVf, IVg, IVh, IVj, IVk, IVl, IVm, IVn, IVo, IVp, IVq, IVr, IVs, IVt, IVu, IVvA, IVvB, IVw, IVx, IVy, Va, Va′, Vb, Vc, Vd, Ve, Vf, Vg, Vh, Vi, Vj, Vk, VIa, IVb, VIc, VId, VIe, VIf, VIg, VIh, Vi, VIi, VII, VIII, IX, X, D-5a, and D-5c, or a pharmaceutically acceptable salt thereof, covalently bound to L.

2. The compound of claim 1, having a Formula A, B, C, D, or E 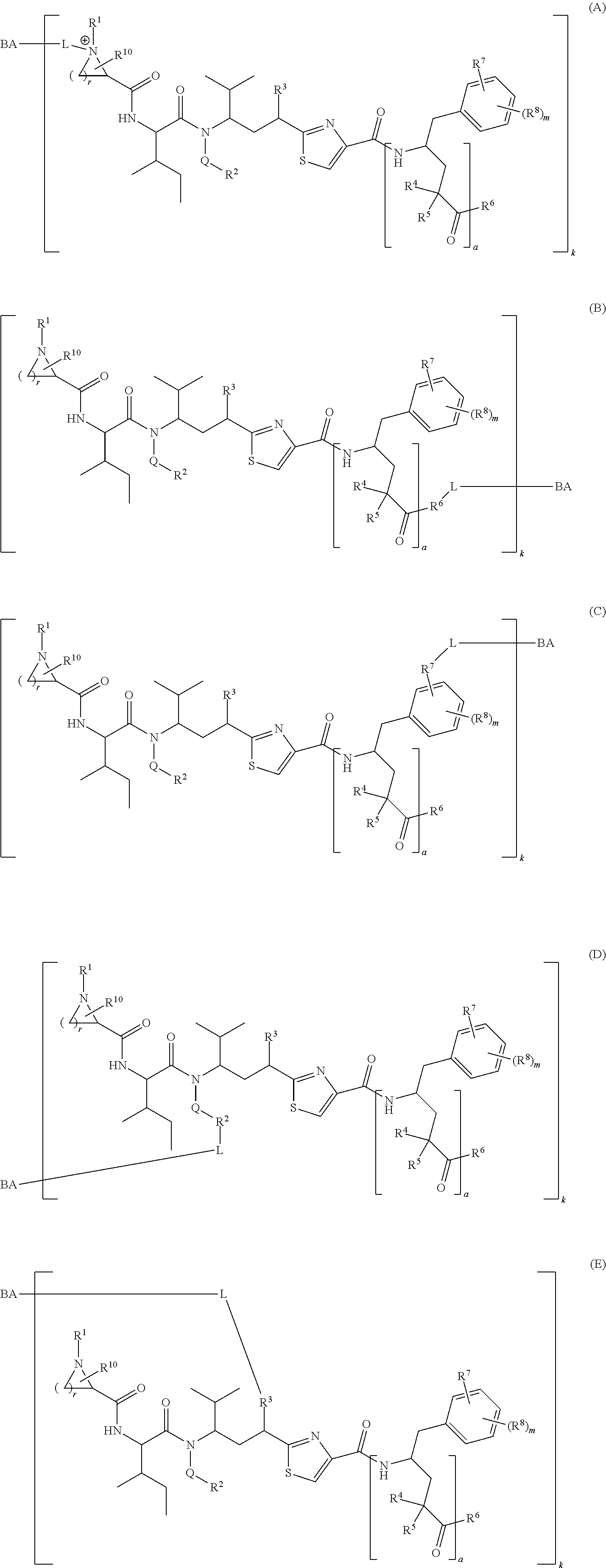
wherein L is a linker.

3. The compound of claim 1, or a pharmaceutically acceptable salt thereof,
wherein
R⁷ is, independently in each instance, hydrogen, —OH, —O—, halogen, or —NR^{7a}R^{7b},
wherein R^{7a} and R^{7b} are, independently in each instance, a bond, hydrogen, alkyl, alkenyl, alkynyl, cycloalkyl, aryl, heteroaryl, acyl, —C(O)CH₂OH, —C(O)CH₂O—, a first N-terminal amino acid residue, a first N-terminal peptide residue, —CH₂CH₂NH₂, and —CH₂CH₂NH—, wherein alkyl, alkenyl, alkynyl, cycloalkyl, aryl, heteroaryl, and acyl are optionally substituted.

4. The compound of claim 2, wherein the compound is of the Formula A′, B′, C′, D′, or E′ 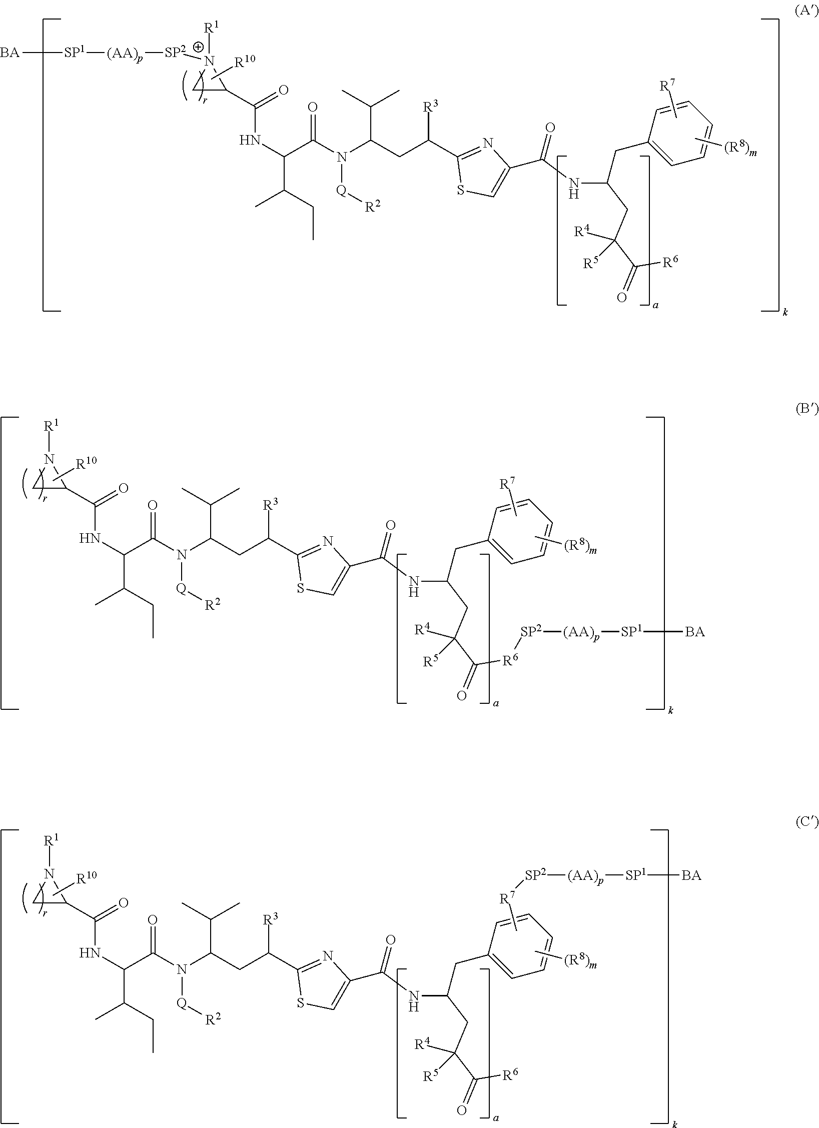 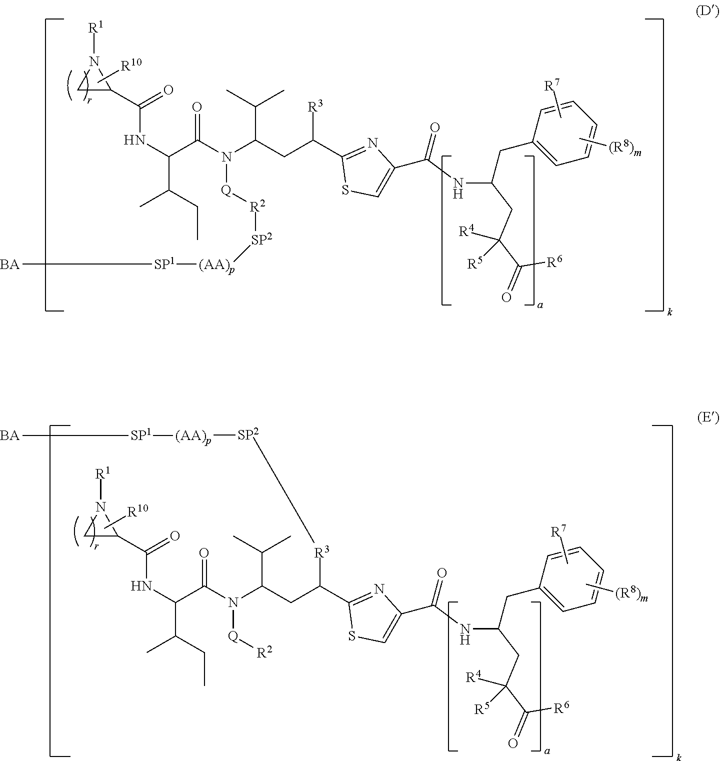
wherein SP¹ and SP², when present, are spacer groups;
each AA, when present, is a second amino acid residue; and
p is an integer from zero to ten.

5. The compound of claim 4, wherein
the -SP²- spacer, when present, is 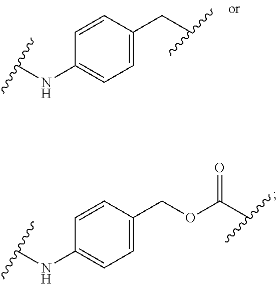 [SMILES: *Cc1ccc(N*)cc1 ; *Nc1ccc(COC(*)=O)cc1]
the second -(AA)ₚ- is 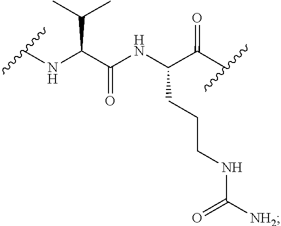 [SMILES: *N[C@H](C(=O)N[C@@H](CCCNC(N)=O)C(*)=O)C(C)C]
the -SP¹- spacer is 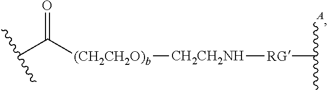
wherein RG′ is a reactive group residue following reaction of a reactive group RG with a binding agent; [symbol]
is a bond, direct or indirect, to the binding agent; and
b is an integer from one to four.

6. The compound of claim 5, wherein the binding agent is an antibody modified with a primary amine compound according to the Formula H₂N-LL-X, wherein LL is a divalent linker selected from the group consisting of
a divalent polyethylene glycol (PEG) group;
—(CH₂)ₙ—;
—(CH₂CH₂O)ₙ—(CH₂)ₚ—;
—(CH₂)ₙ—N(H)C(O)—(CH₂)ₘ—;
—(CH₂CH₂O)ₙ—N(H)C(O)—(CH₂CH₂O)ₘ—(CH₂)ₚ—;
—(CH₂)ₙ—C(O)N(H)—(CH₂)ₘ—;
—(CH₂CH₂O)ₙ—C(O)N(H)—(CH₂CH₂O)ₘ—(CH₂)ₚ—;
—(CH₂)ₙ—N(H)C(O)—(CH₂CH₂O)ₘ—(CH₂)ₚ—;
—(CH₂CH₂O)ₙ—N(H)C(O)—(CH₂)ₘ—;
—(CH₂)ₙ—C(O)N(H)—(CH₂CH₂O)ₘ—(CH₂)ₚ—; and
—(CH₂CH₂O)ₙ—C(O)N(H)—(CH₂)ₘ—,
wherein
n is an integer selected from one to twelve;
m is an integer selected from zero to twelve;
p is an integer selected from zero to two; and
X is selected from the group consisting of —SH, —N₃, —C≡C_{H}, —C(O)H, tetrazole,

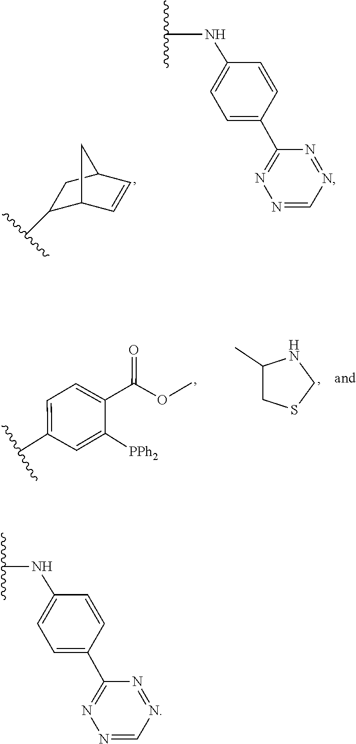

7. The compound of claim 6, wherein the binding agent is an antibody modified with a primary amine according to the following formula

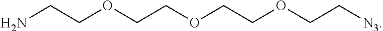 [SMILES: [N-]=[N+]=NCCOCCOCCOCCN]

8. The compound of claim 4, wherein Q is —O—.

9. The compound of claim 4, wherein
Q is —CH₂—;
R¹ is C₁-C₁₀ alkyl;
R² is alkyl;
R⁴ and R⁵ are C₁-C₅ alkyl;
R⁶ is —OH;
R¹⁰ is absent;
wherein r is four; and
wherein a is one.

10. The compound of claim 9, according to the structure of C′, or a pharmaceutically acceptable salt thereof.

11. The compound of claim 10, wherein R⁷ is —NH—; and R⁸ is hydrogen or fluoro.

12. The compound of claim 9, according to the structure of E′, or a pharmaceutically acceptable salt thereof.

13. The compound of claim 12, wherein R³ is —OC(O)N(H)CH₂CH₂NH—or —OC(O)N(H)CH₂CH₂OCH₂CH₂OCH₂CH₂OCH₂CH₂NH—.

14. The compound of claim 4, wherein
Q is —CH₂—;
R¹ is hydrogen or C₁-C₁₀ alkyl;
R² is alkyl;
R⁴ and R⁵ are C₁-C₅ alkyl;
R⁶ is —OH;
wherein r is three or four; and
wherein a is one.

15. The compound of claim 14, according to the structure of C′, or a pharmaceutically acceptable salt thereof.

16. The compound of claim 15, wherein R⁷ is —NH—; and R⁸ is hydrogen.

17. The compound of claim 4, wherein
Q is —CH₂—;
R¹ is hydrogen or C₁-C₁₀ alkyl;
R² is alkyl;
R⁴ and R⁵ are C₁-C₅ alkyl;
R⁶ is —OH;
R¹⁰ is absent;
wherein r is four; and
wherein a is one.

18. The compound of claim 17, according to the structure of C′, or a pharmaceutically acceptable salt thereof.

19. The compound of claim 18, wherein R⁷ is —NH—; and R⁸ is hydrogen.

20. The compound of claim 4, wherein
Q is —O—;
R¹ is hydrogen or C₁-C₁₀ alkyl;
R² is alkyl or alkynyl;
R³ is hydroxyl or —OC(O)C₁-C₅ alkyl;
R⁴ and R⁵ are C₁-C₅ alkyl;
R⁶ is —OH;
R¹⁰, when present, is —C₁-C₅ alkyl;
wherein r is three or four; and
wherein a is one.

21. The compound of claim 20, according to the structure of C′, or a pharmaceutically acceptable salt thereof.

22. The compound of claim 21, wherein R⁷ is —NH—; and R⁸ is hydrogen.

23. The compound of claim 4, wherein
Q is —CH₂— or —O—;
R¹ is C₁-C₁₀ alkyl;
R² is alkyl or alkynyl;
R⁴ and R⁵ are C₁-C₅ alkyl;
R⁶ is —NHSO₂(CH₂)ₐ₁-aryl-(CH₂)ₐ₂NR^{6a}R^{6b};
R¹⁰ is absent;
wherein r is four; and
wherein a, a1, and, a2 are, independently, zero or one.

24. The compound of claim 23, according to the structure of B′, or a pharmaceutically acceptable salt thereof.

25. The compound of claim 24, wherein R⁶ is

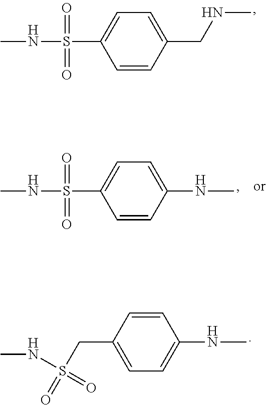

26. The compound of claim 24, wherein a is zero; and R⁶ is

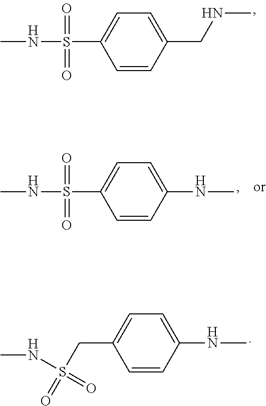

27. The compound of claim 24, wherein a is one; and R⁶ is

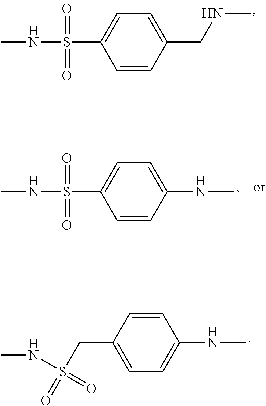

28. The compound of claim 21, wherein R⁷ is —O—; and R⁸ is hydrogen.

29. The compound of claim 4, selected from the group consisting of 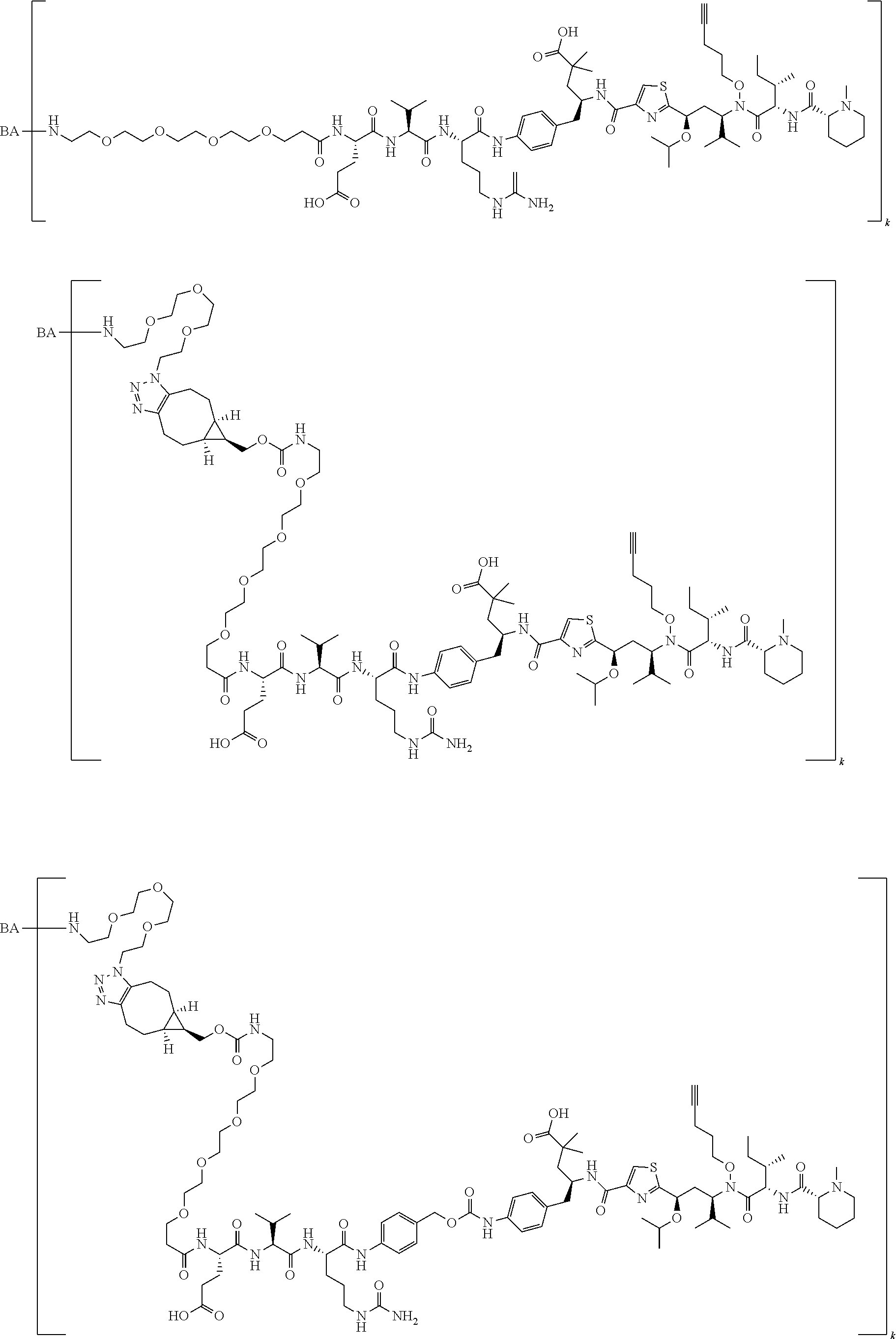 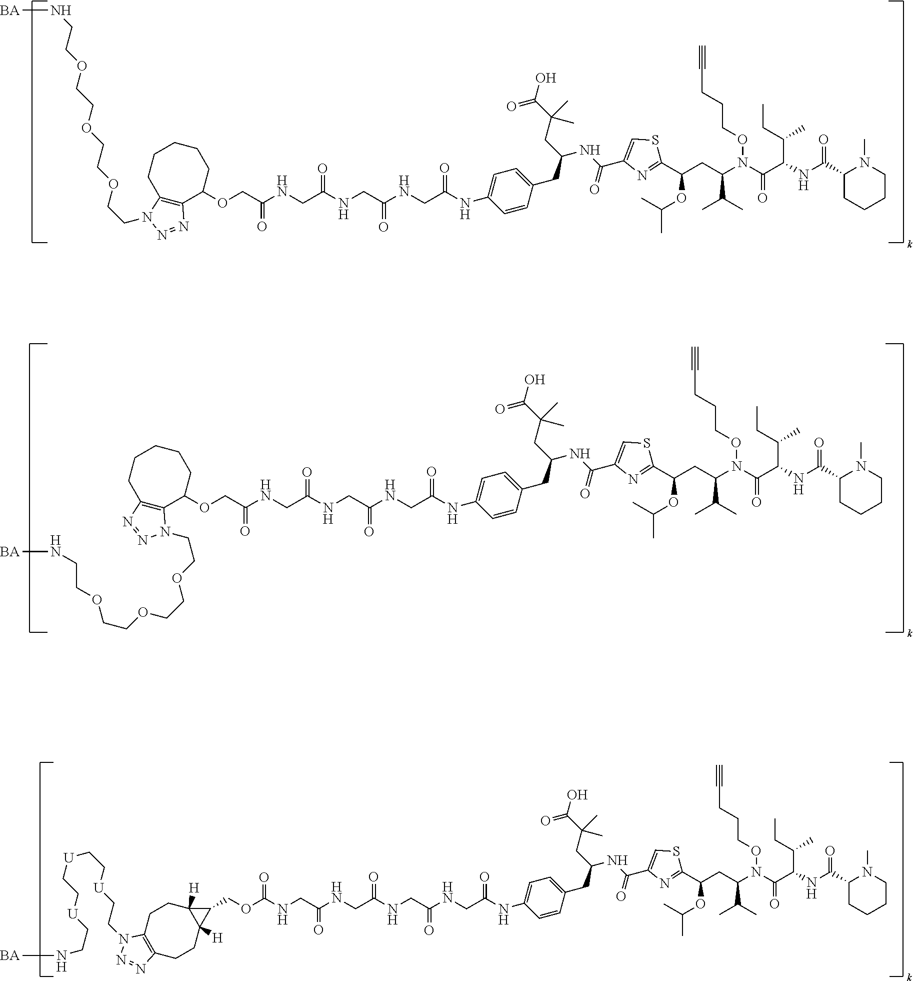 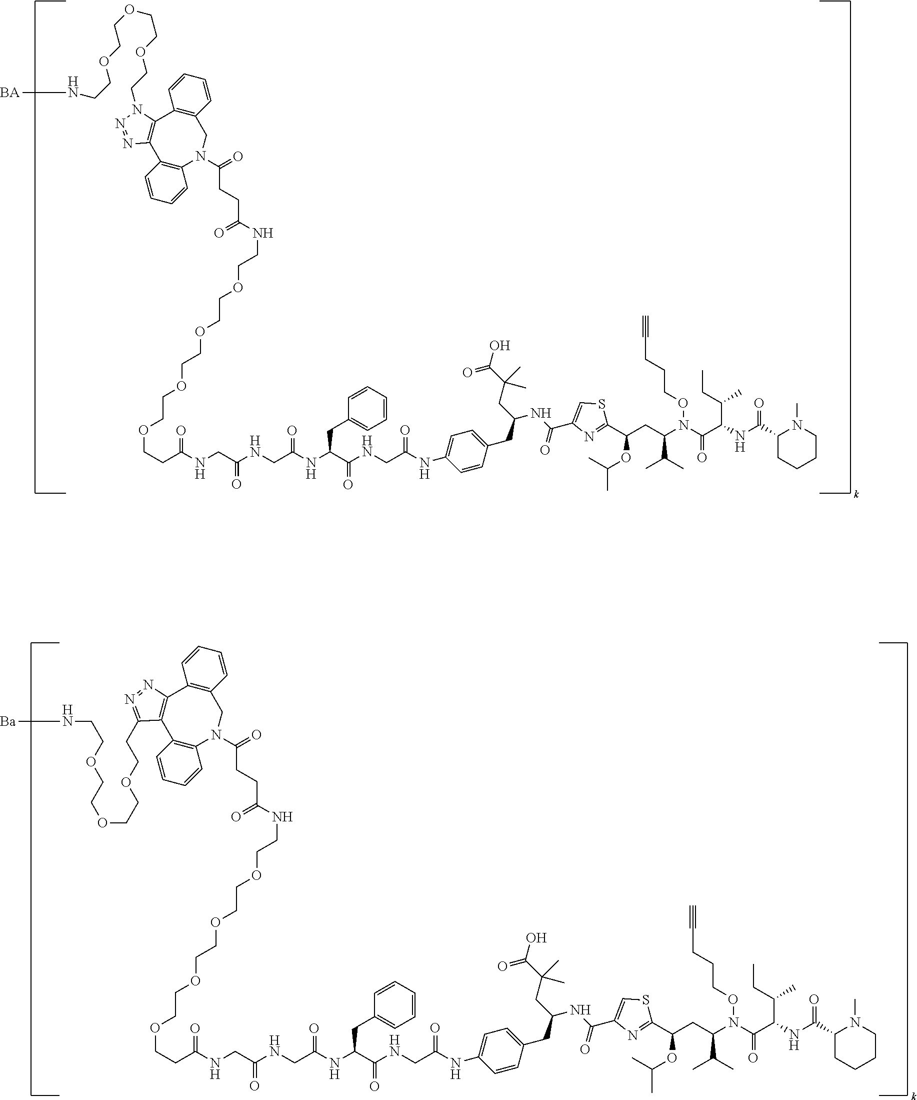 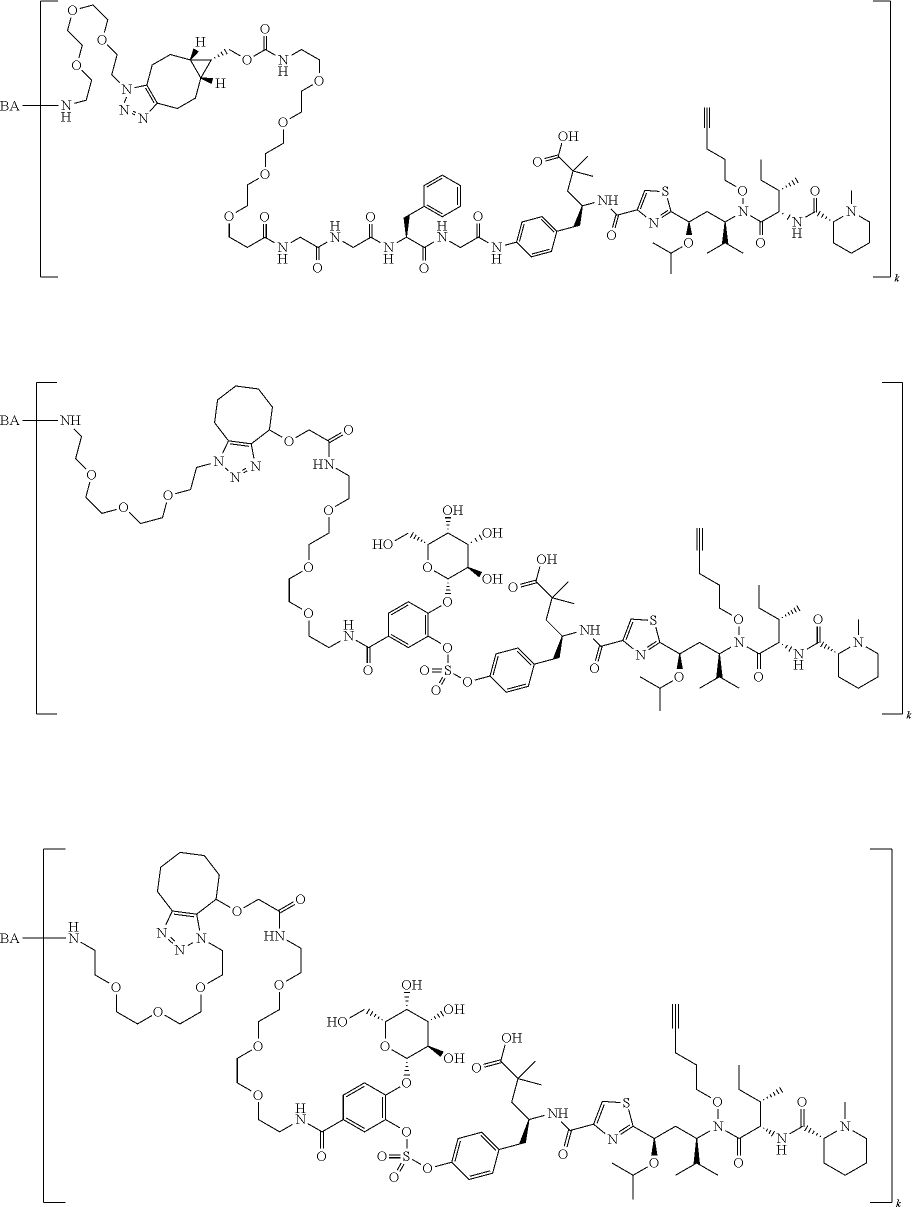 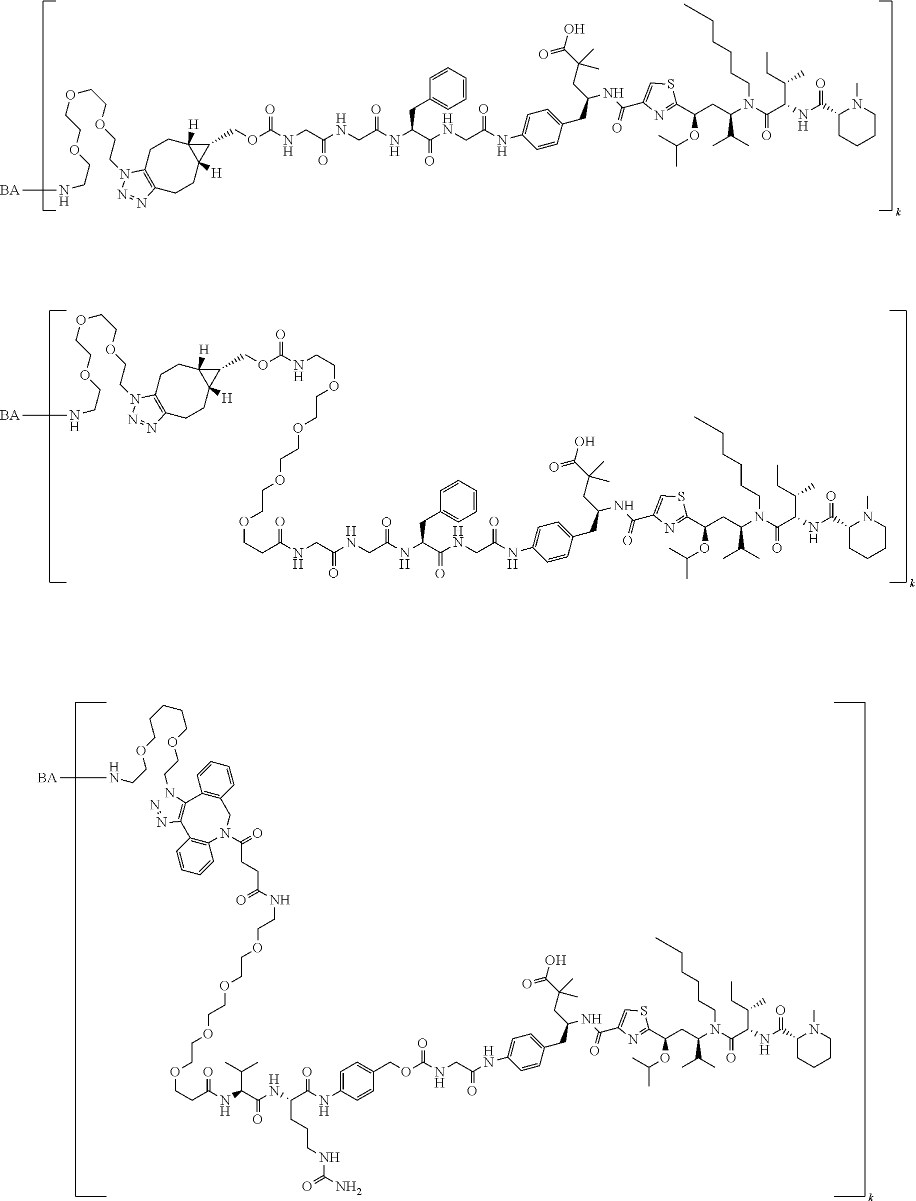 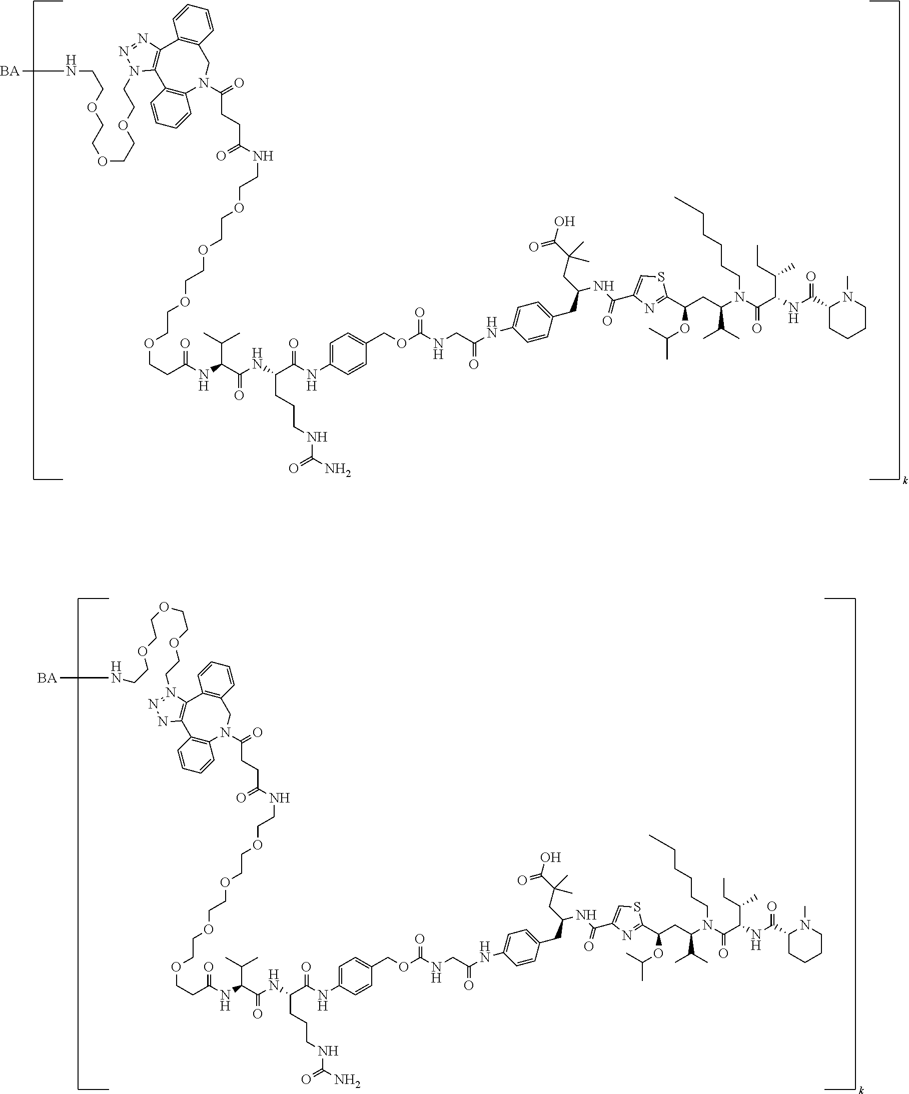 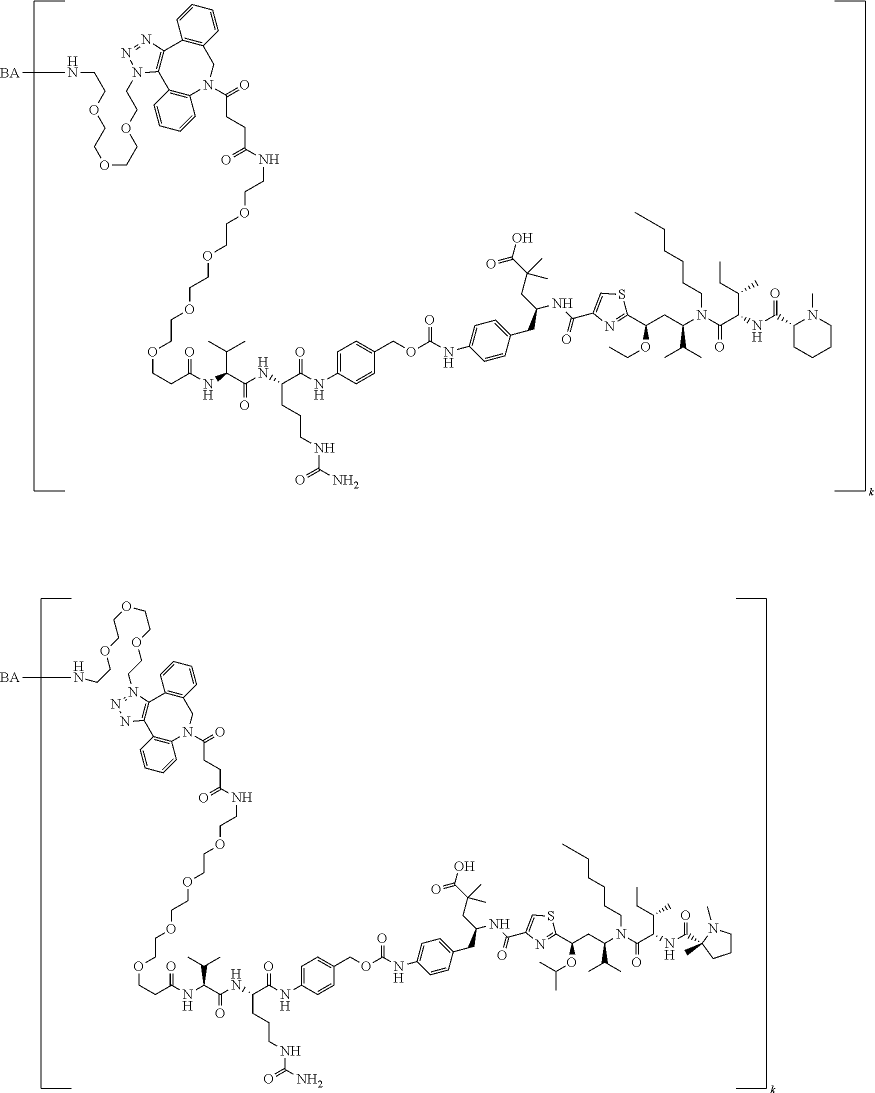 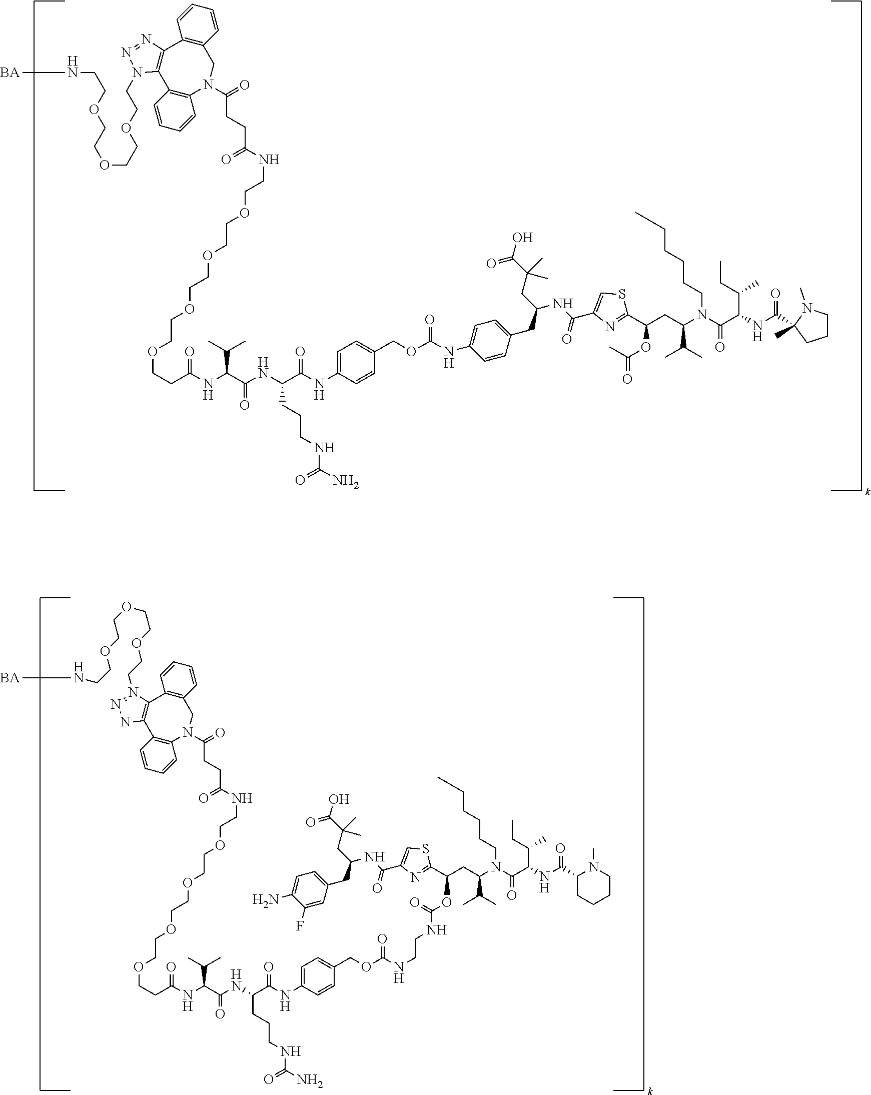 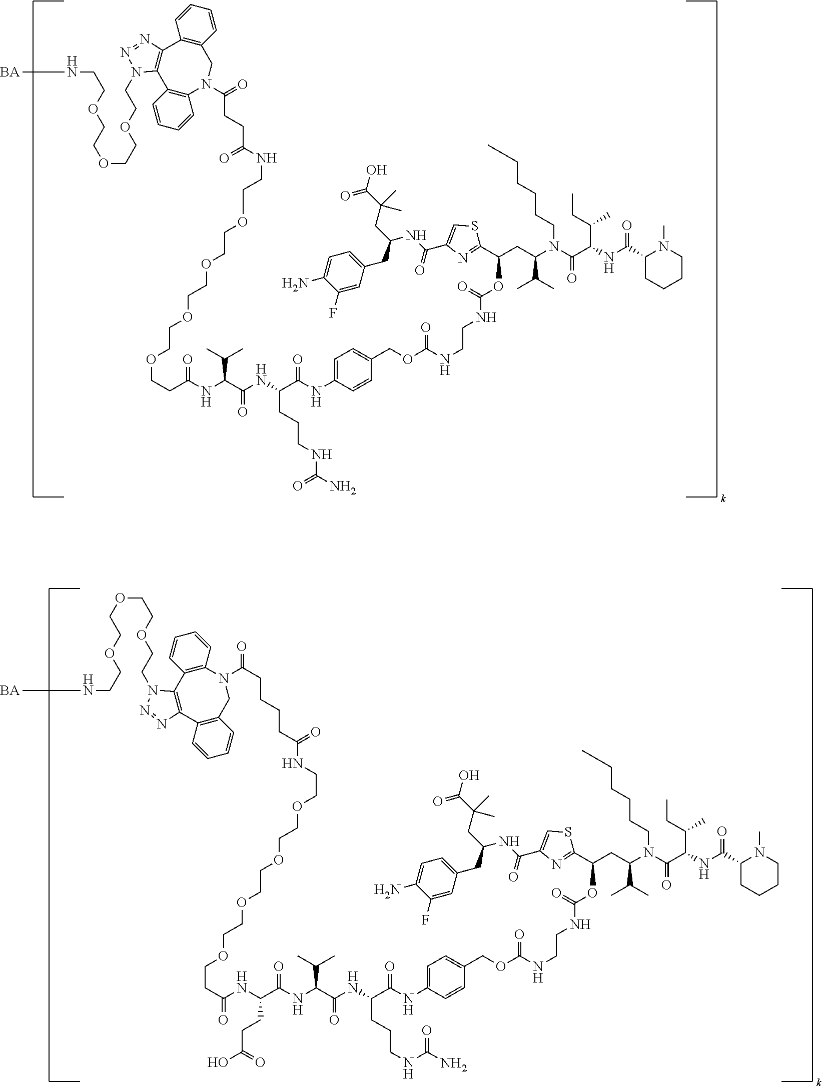 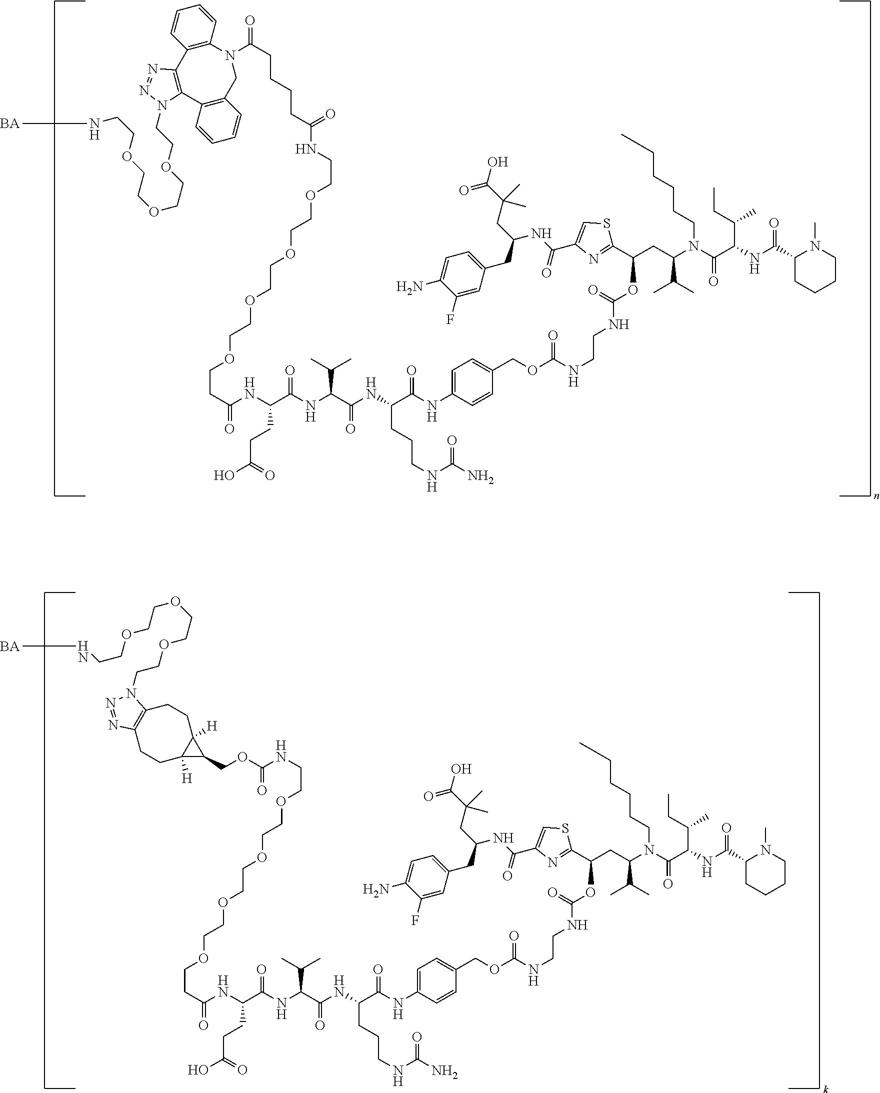 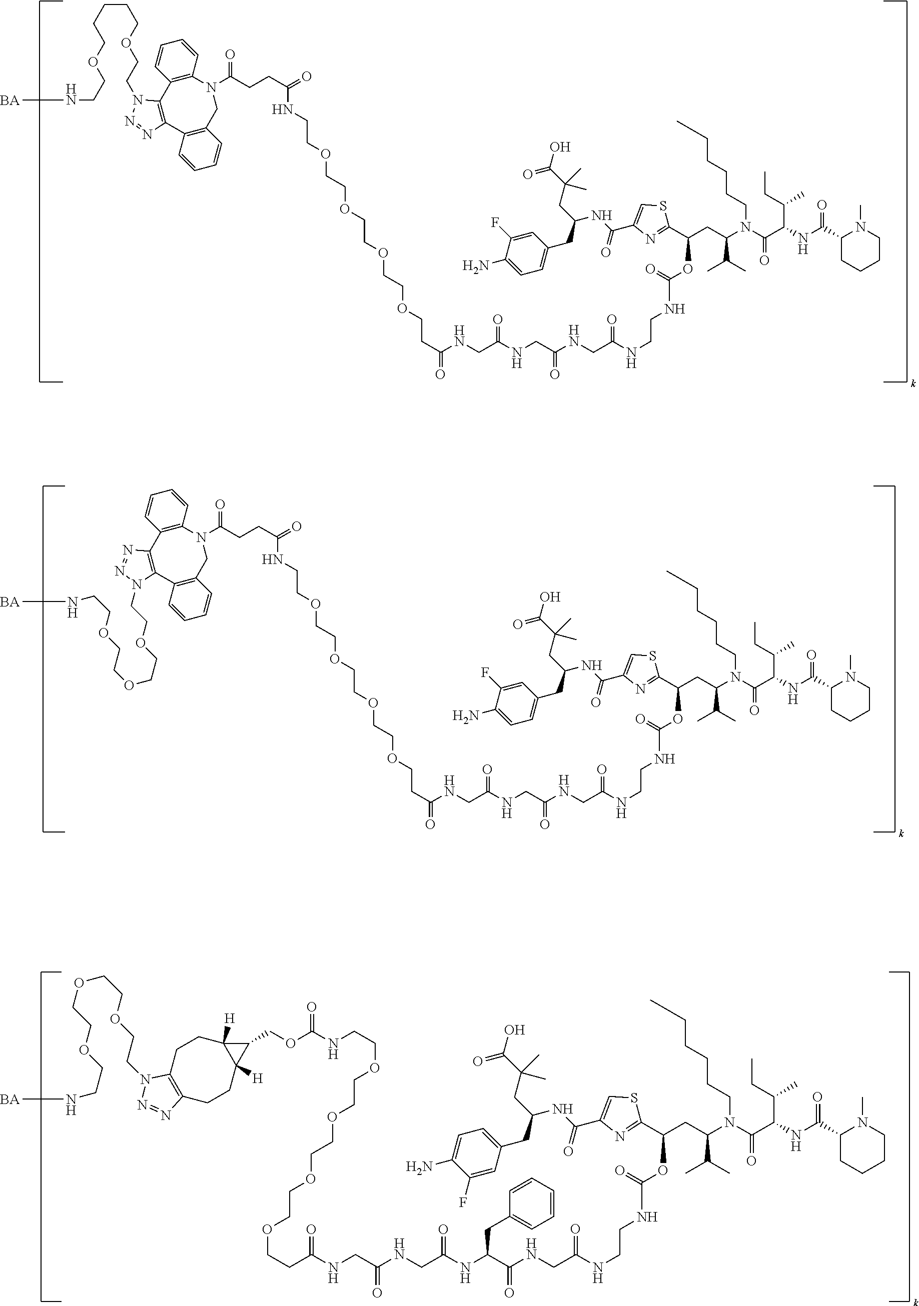 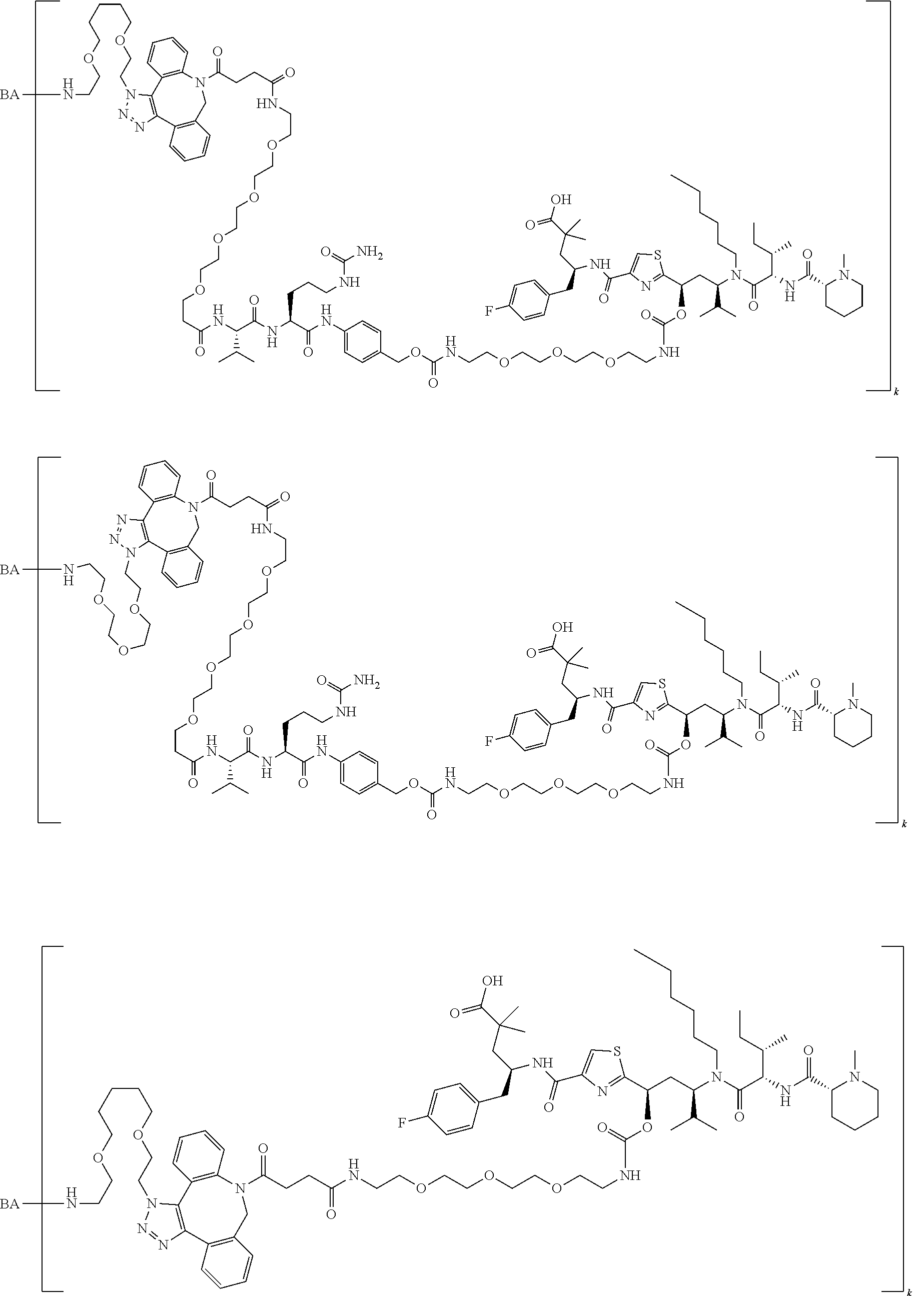 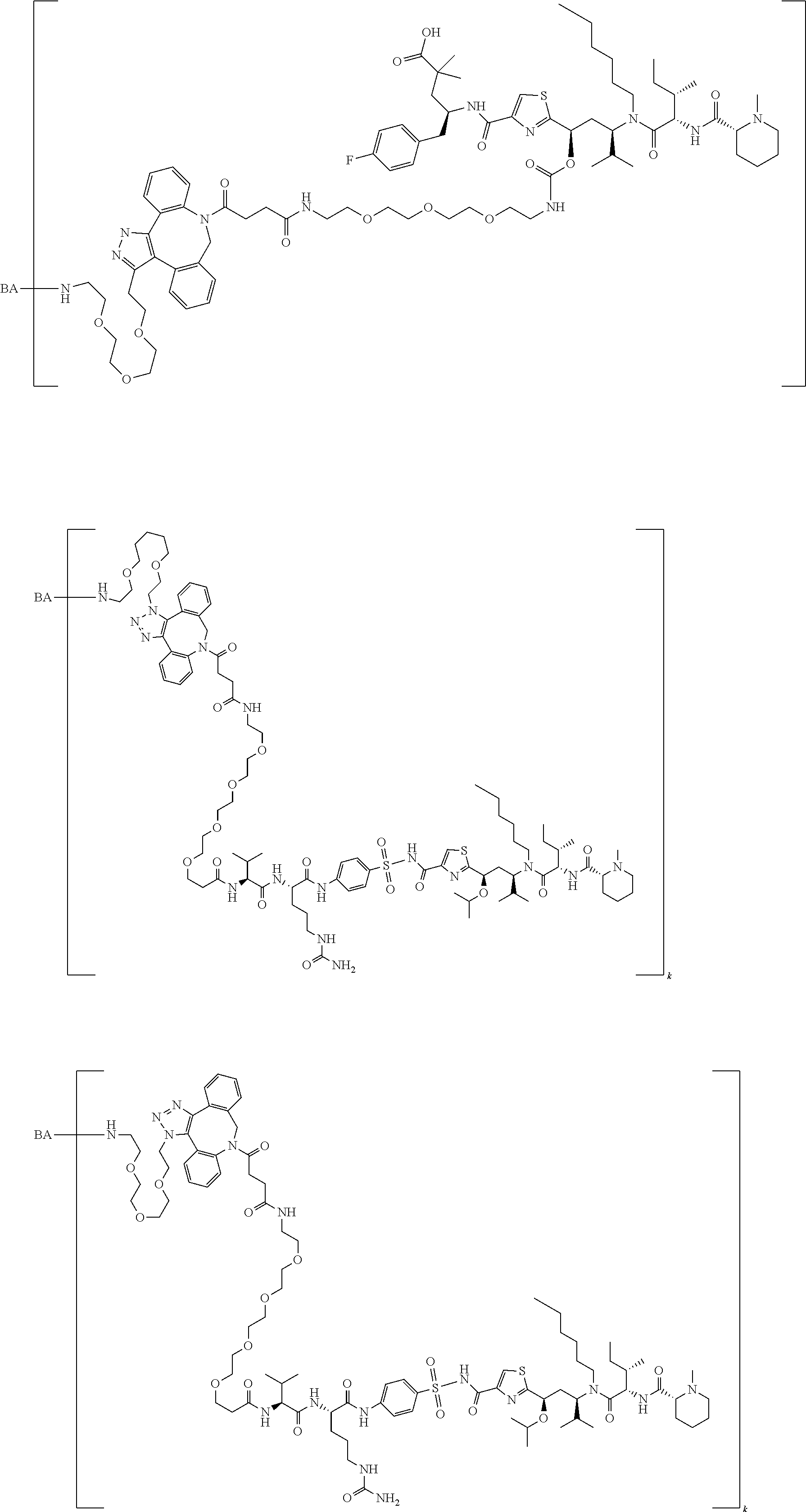 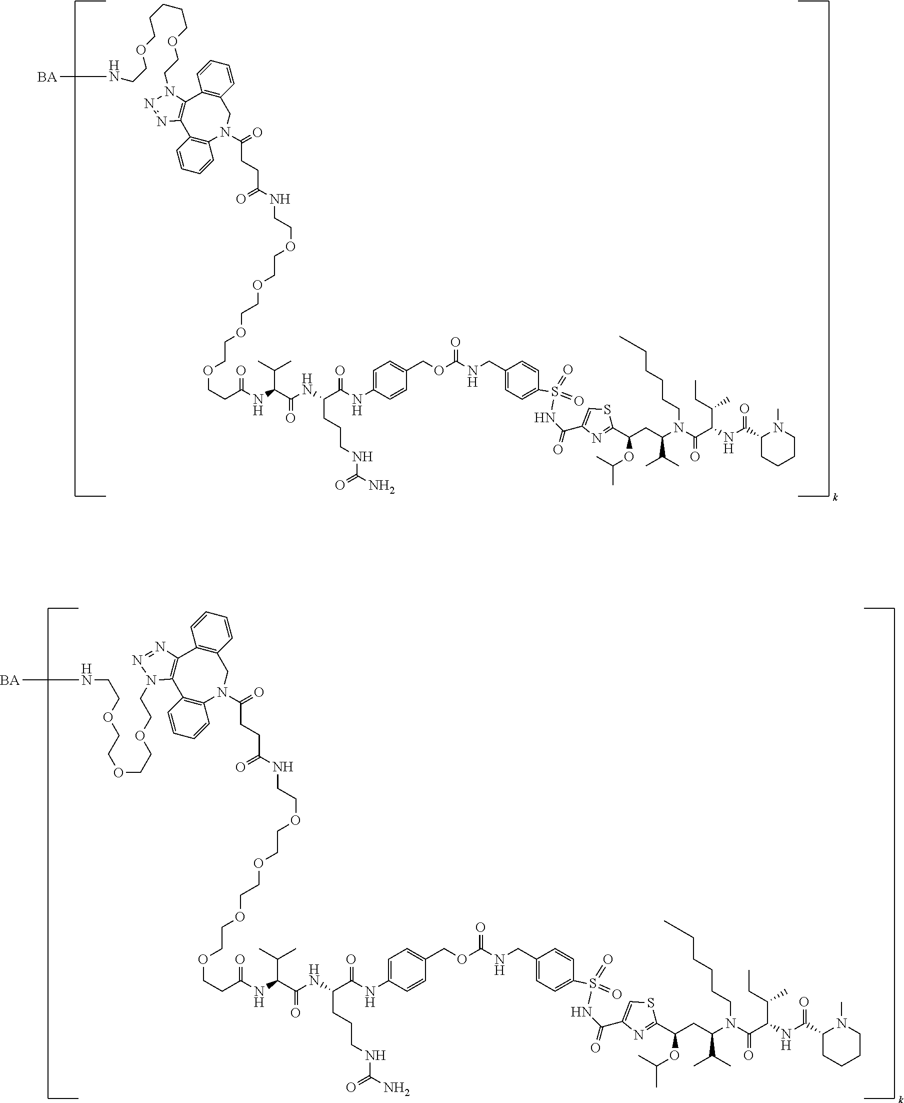 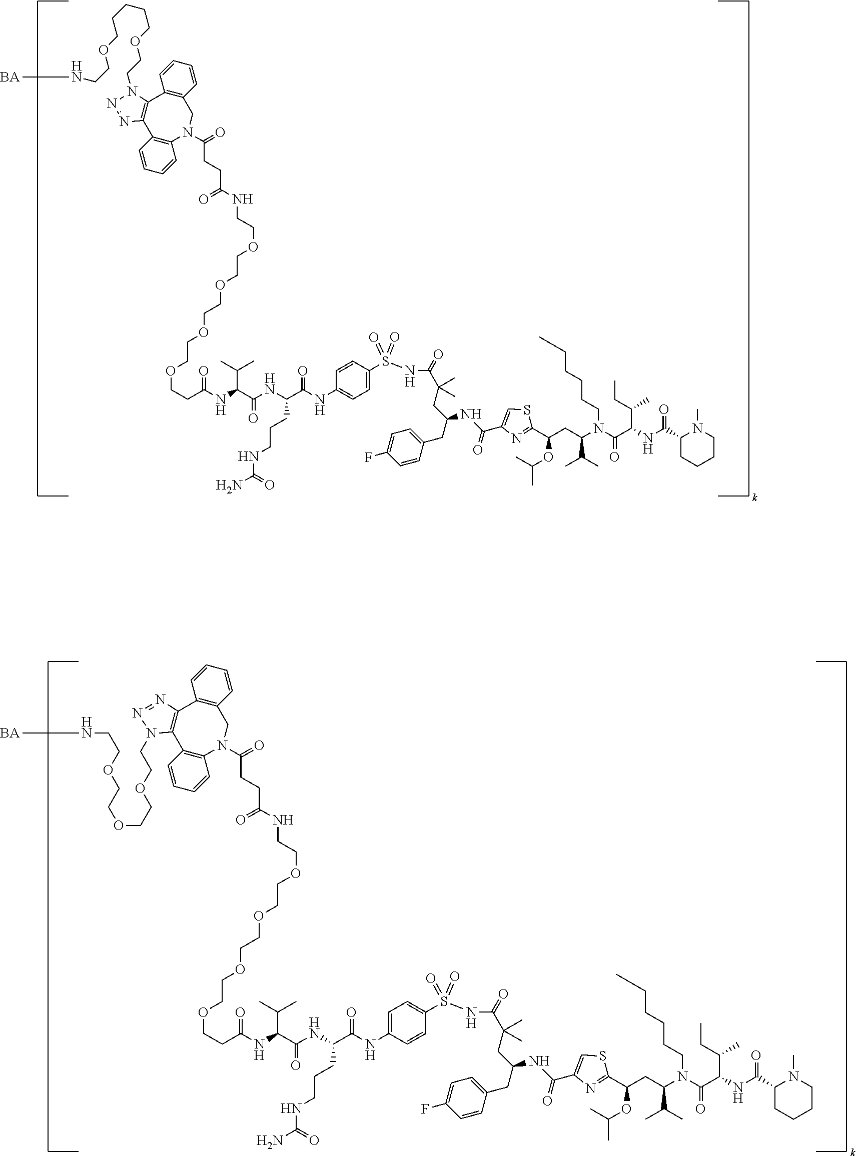 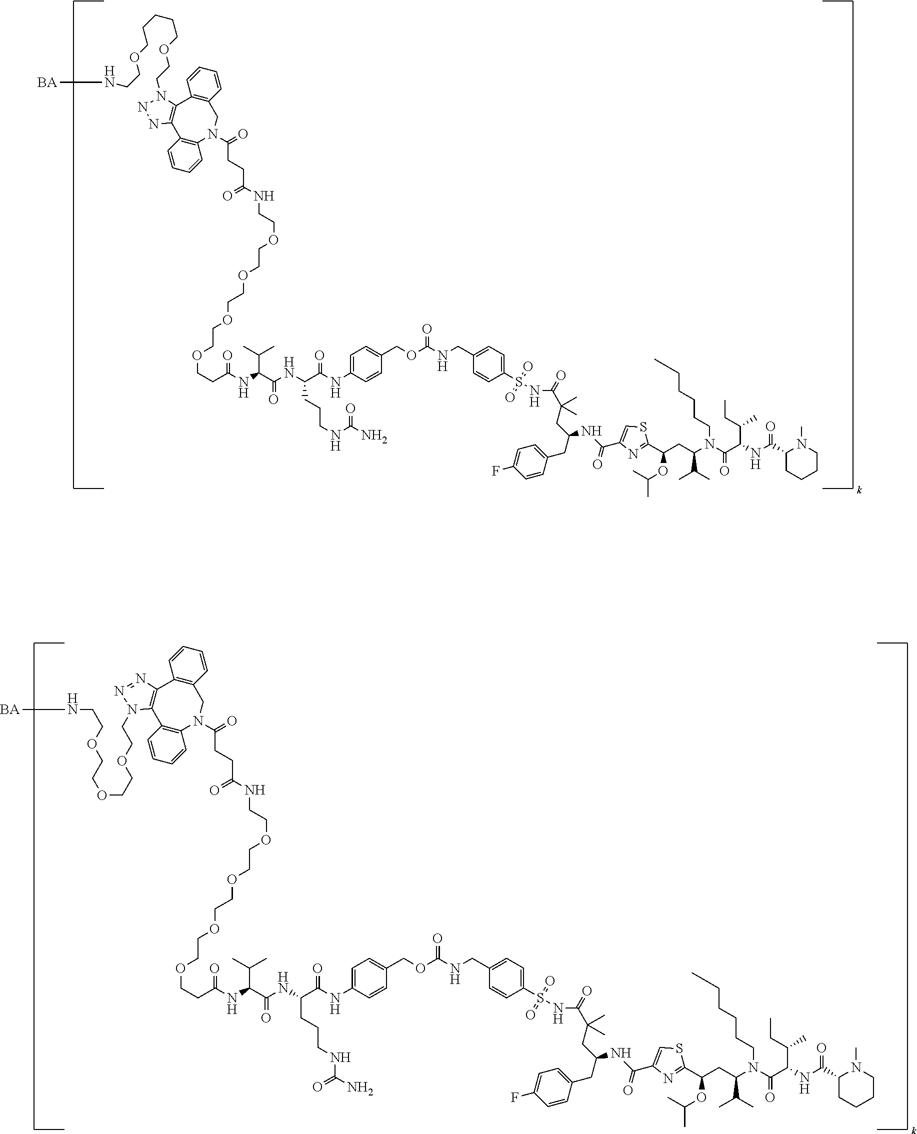 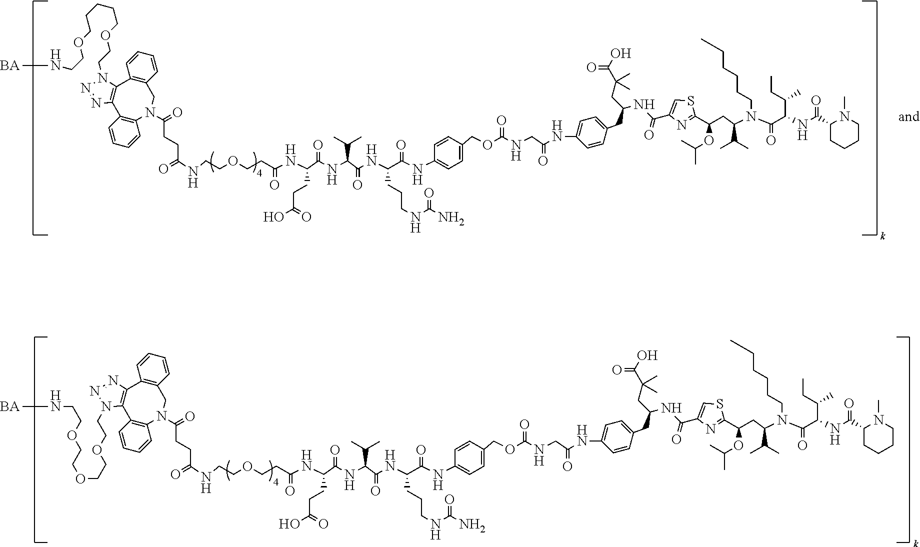
or a pharmaceutically acceptable salt thereof,
wherein BA is a binding agent; and k is one, two, three, or four.

30. The compound of claim 29, wherein BA is an antibody or antigen-binding fragment thereof.

31. The compound of claim 29, wherein BA is a transglutaminase-modified antibody or antigen-binding fragment thereof comprising at least one glutamine residue used for conjugation.

32. The compound of claim 29, wherein BA is a transglutaminase-modified antibody or antigen-binding fragment thereof comprising at least two glutamine residues used for conjugation.

33. The compound of claim 29, wherein BA is a transglutaminase-modified antibody or antigen-binding fragment thereof comprising at least four glutamine residues used for conjugation.

34. The compound of claim 33, wherein BA is a transglutaminase-modified antibody or antigen-binding fragment thereof wherein conjugation is at two Q295 residues; and k is two.

35. The compound of claim 33, wherein BA is a transglutaminase-modified antibody or antigen-binding fragment thereof wherein conjugation is at two Q295 residues and two N297Q residues; and k is four.

36. The compound of claim 1, wherein the compound is an antibody-drug conjugate comprising an antibody or antigen-binding fragment thereof conjugated to a compound selected from the group consisting of

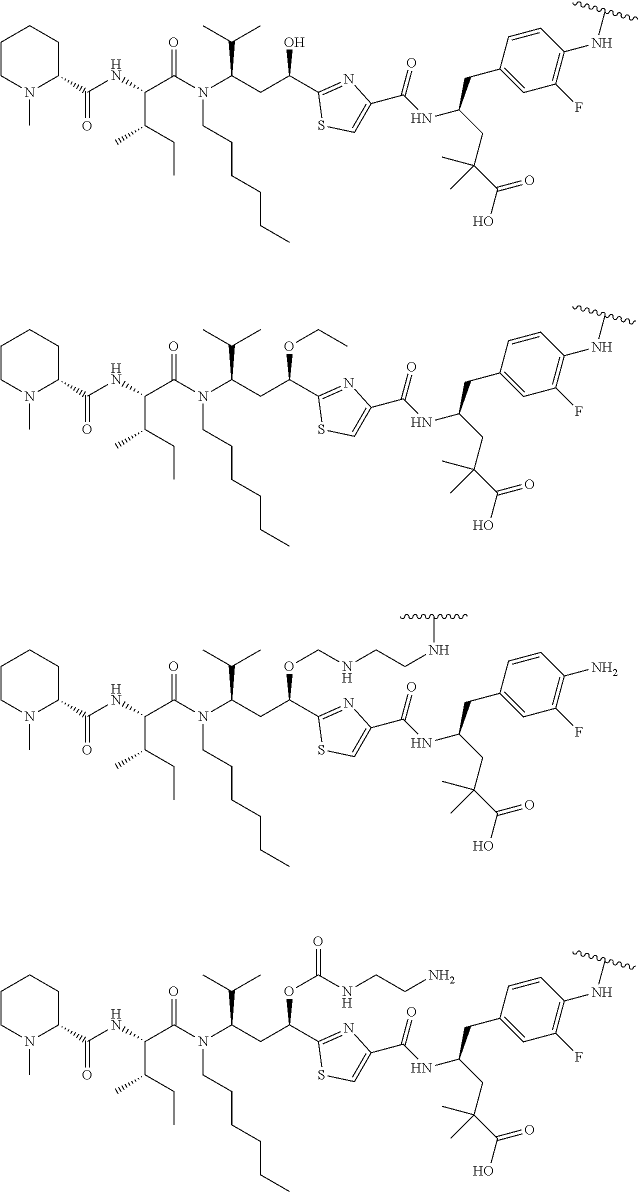

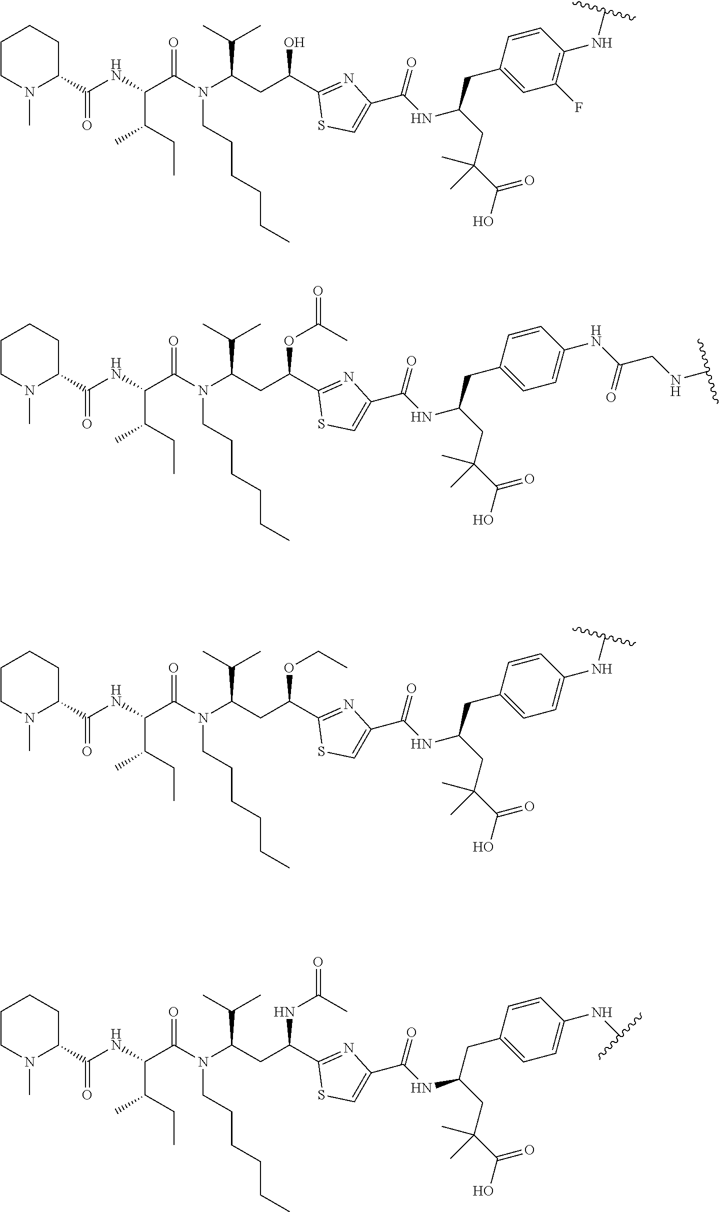

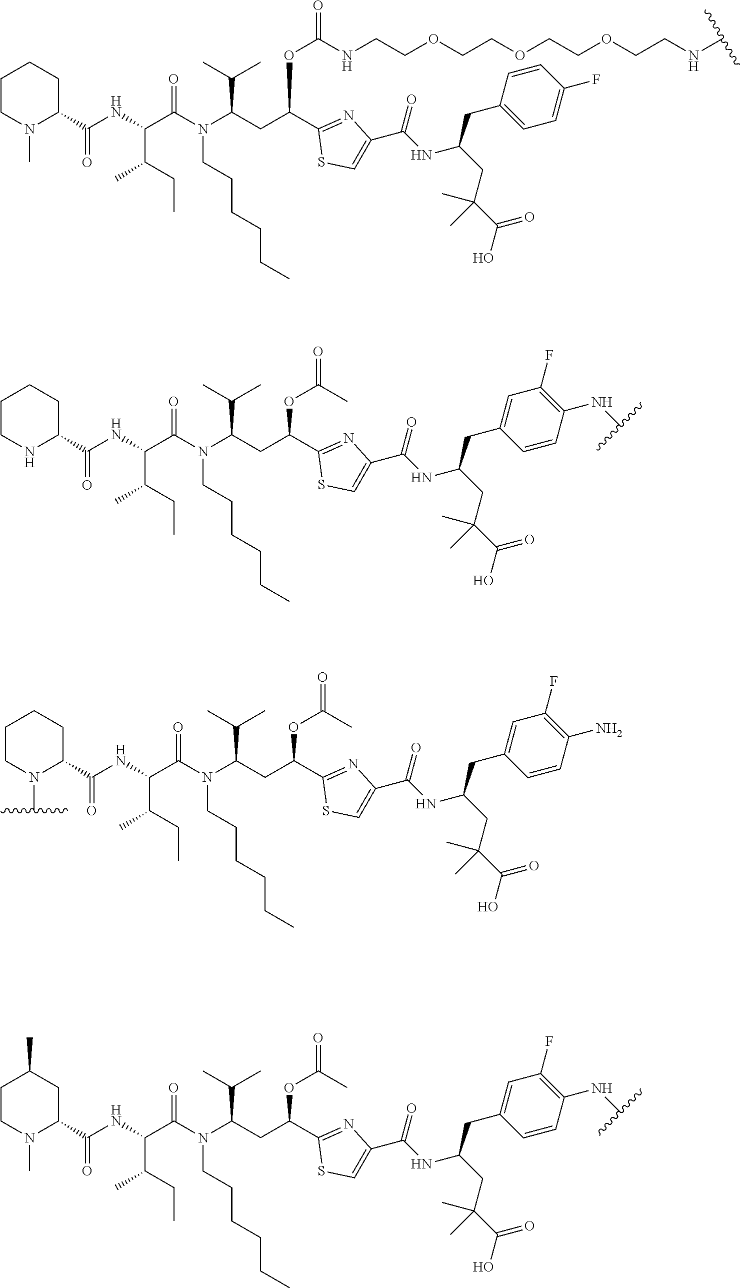

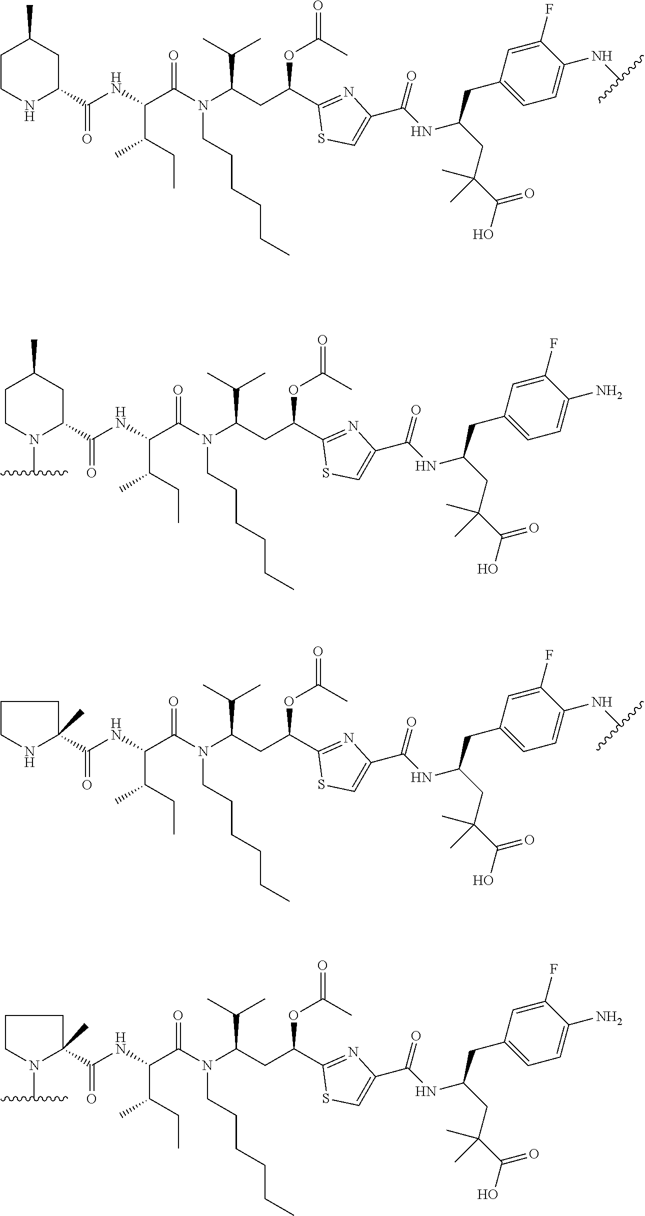

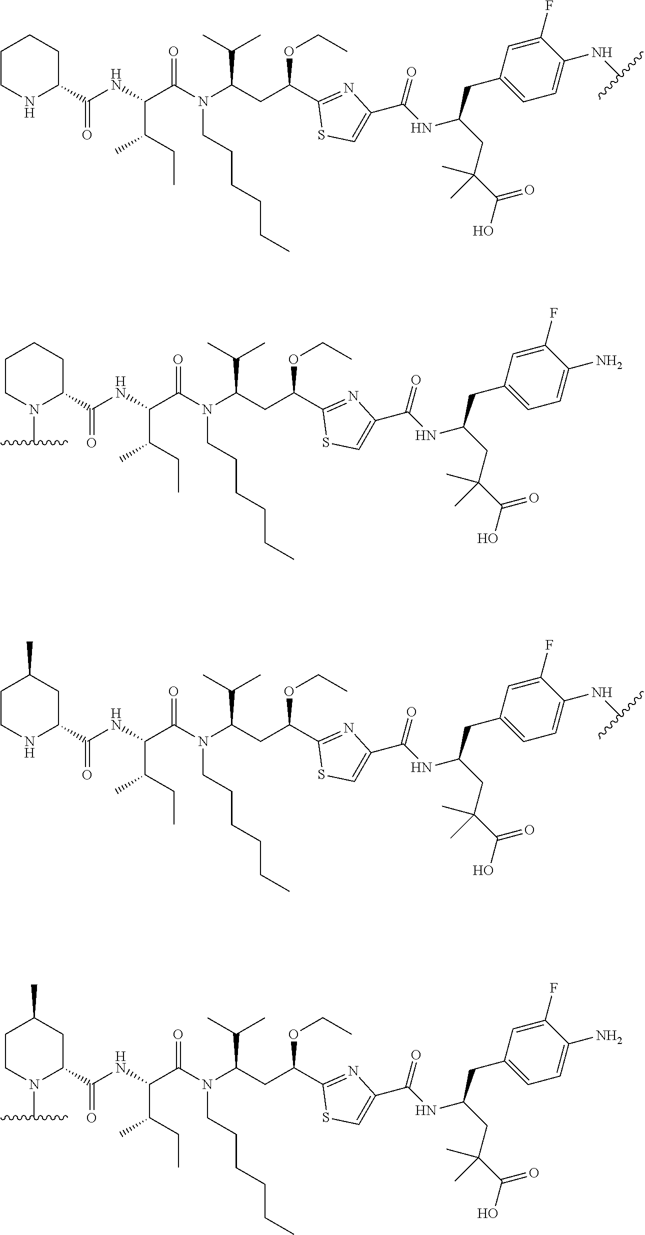

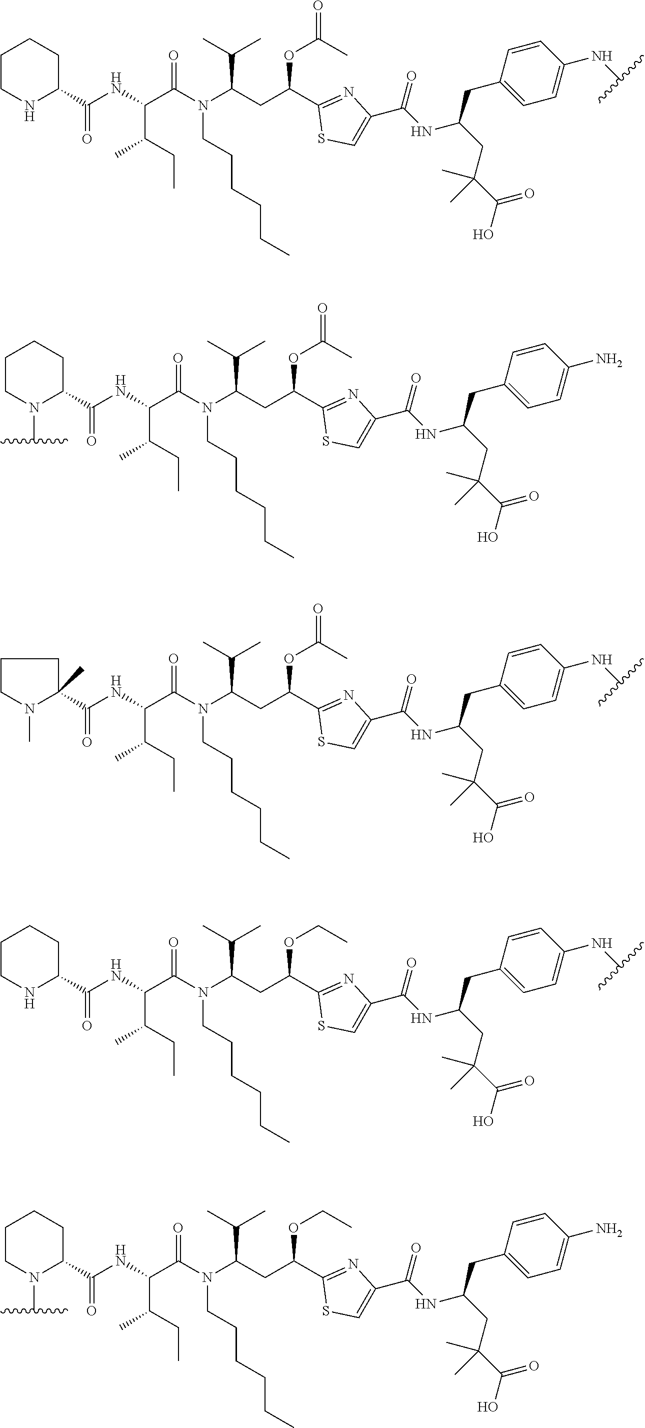

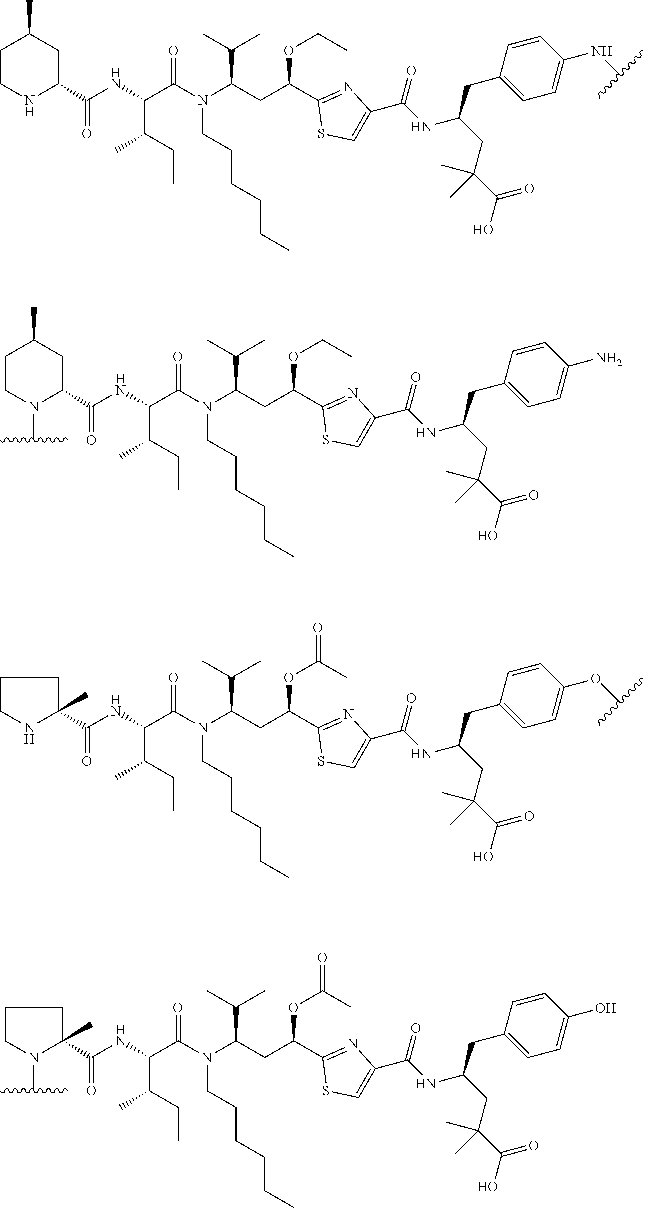

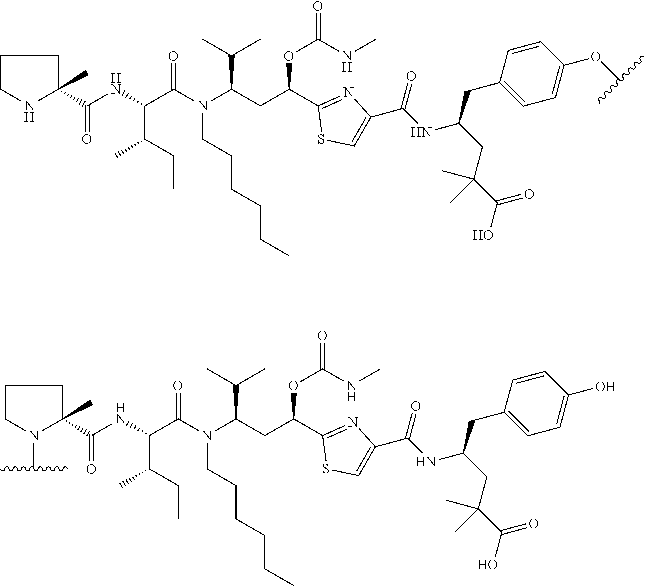 [SMILES: *Oc1ccc(C[C@@H](CC(C)(C)C(=O)O)NC(=O)c2csc([C@@H](C[C@H](C(C)C)N(CCCCCC)C(=O)[C@@H](NC(=O)[C@@]3(C)CCCN3)[C@@H](C)CC)OC(=O)NC)n2)cc1 ; *N1CCC[C@]1(C)C(=O)N[C@H](C(=O)N(CCCCCC)[C@H](C[C@@H](OC(=O)NC)c1nc(C(=O)N[C@@H](Cc2ccc(O)cc2)CC(C)(C)C(=O)O)cs1)C(C)C)[C@@H](C)CC]

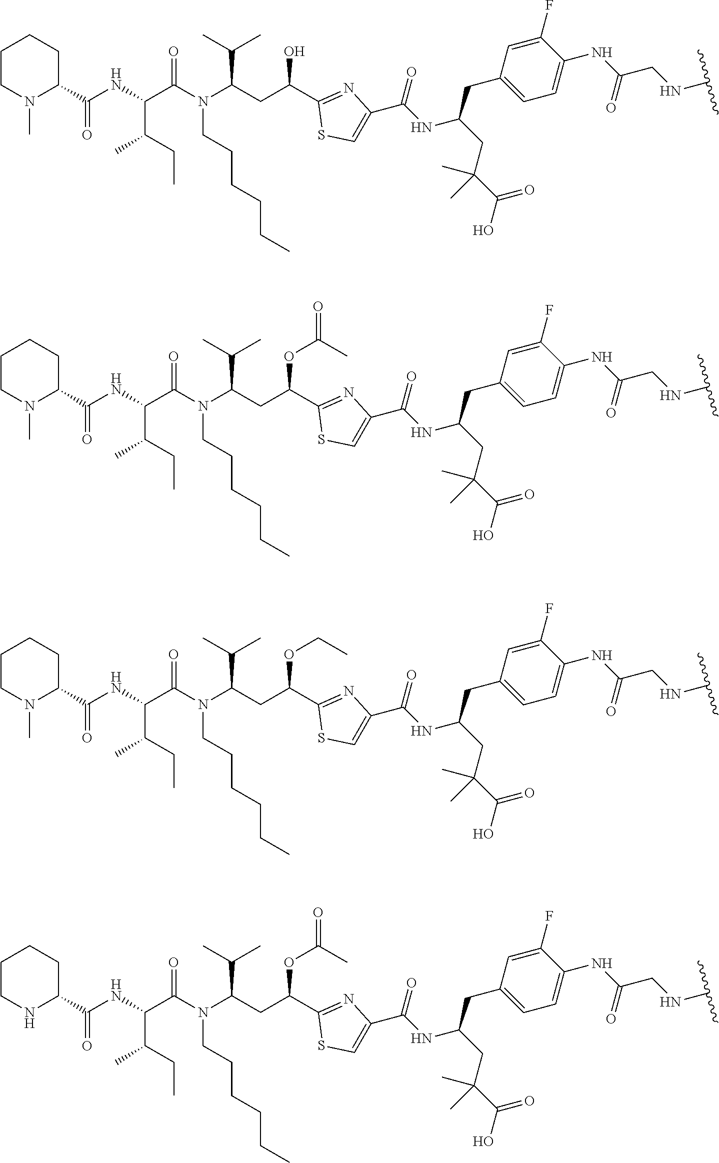

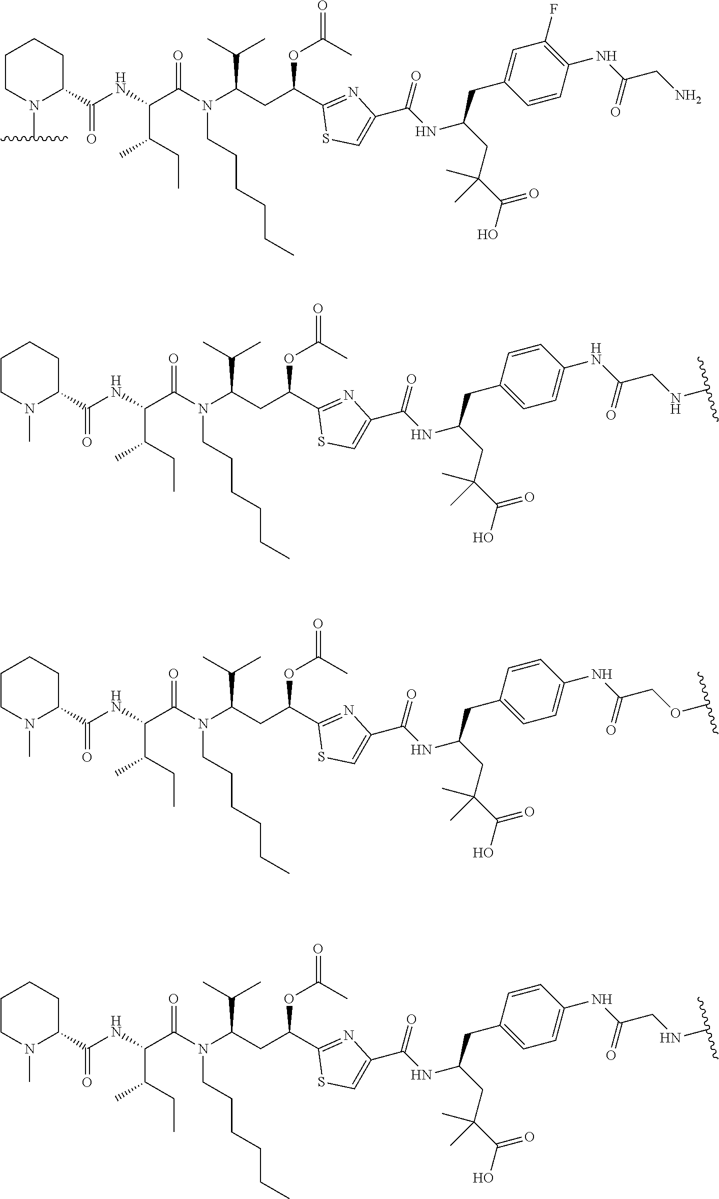

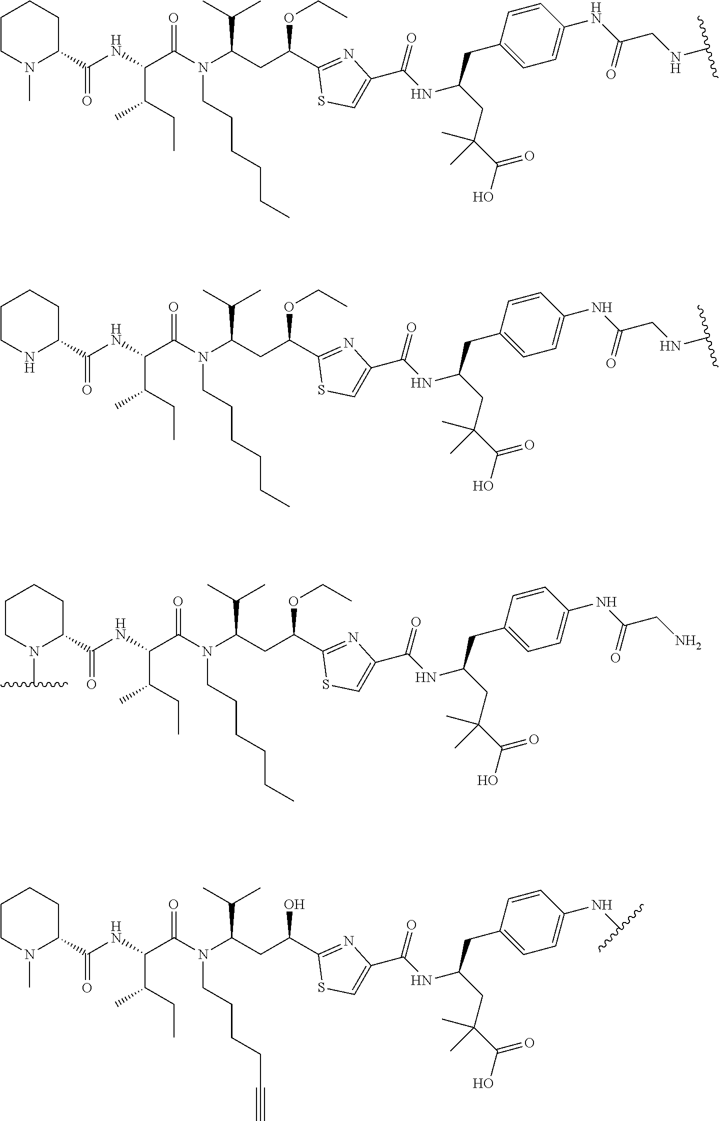

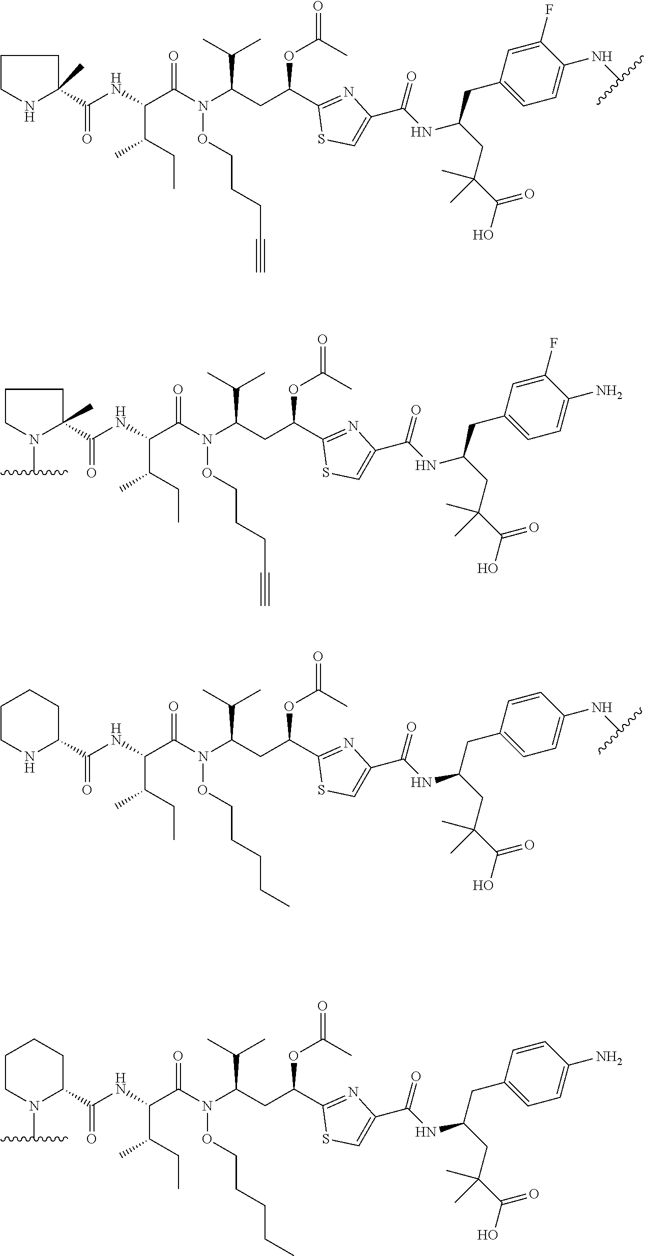

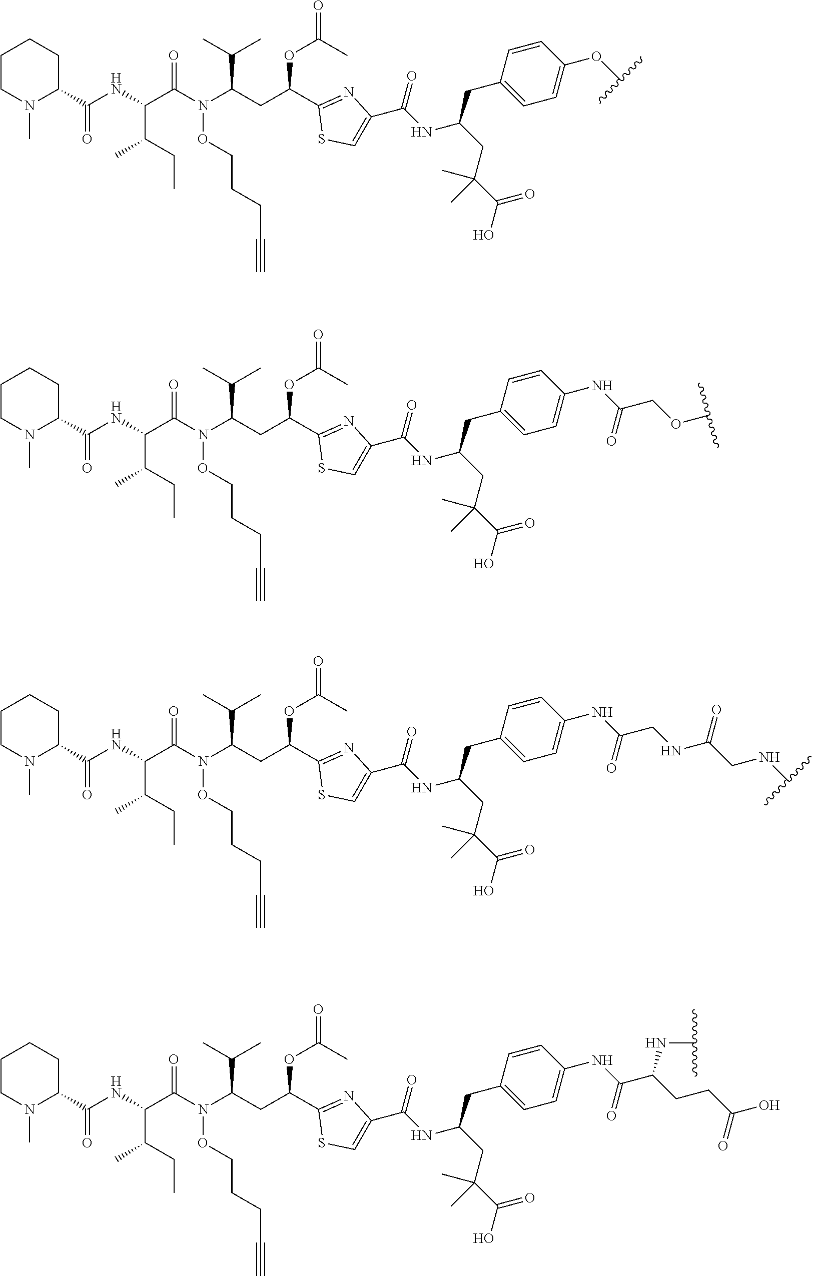

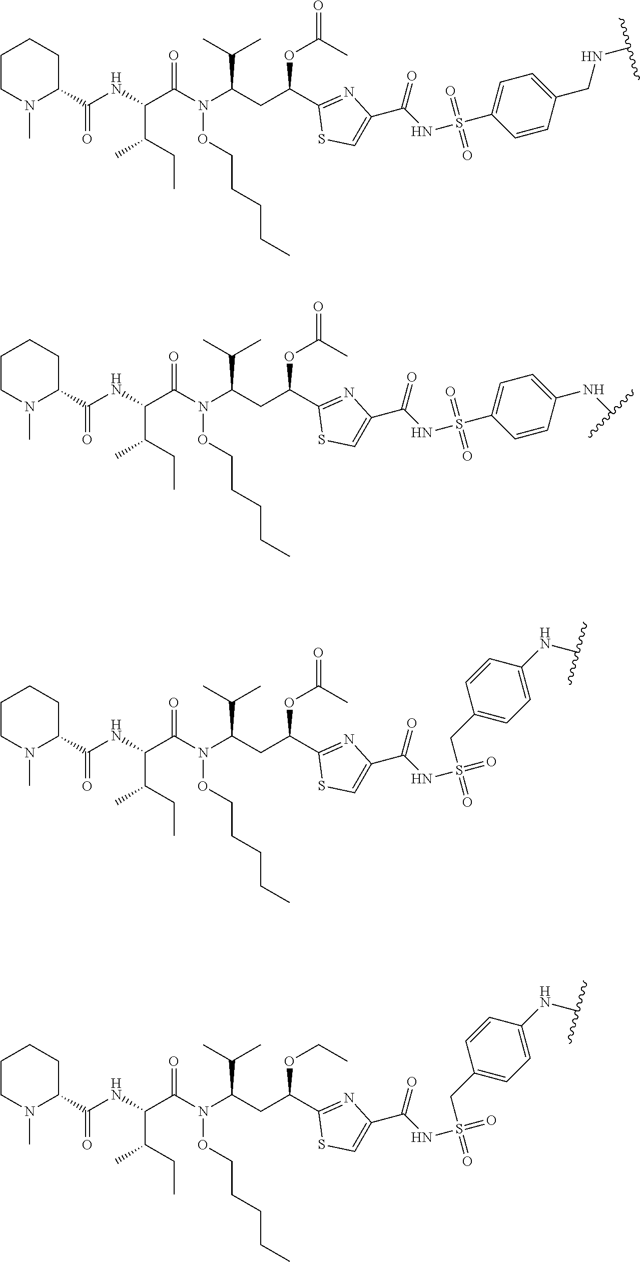 [SMILES: *NCc1ccc(S(=O)(=O)NC(=O)c2csc([C@@H](C[C@H](C(C)C)N(OCCCCC)C(=O)[C@@H](NC(=O)[C@H]3CCCCN3C)[C@@H](C)CC)OC(C)=O)n2)cc1 ; *Nc1ccc(S(=O)(=O)NC(=O)c2csc([C@@H](C[C@H](C(C)C)N(OCCCCC)C(=O)[C@@H](NC(=O)[C@H]3CCCCN3C)[C@@H](C)CC)OC(C)=O)n2)cc1 ; *Nc1ccc(CS(=O)(=O)NC(=O)c2csc([C@@H](C[C@H](C(C)C)N(OCCCCC)C(=O)[C@@H](NC(=O)[C@H]3CCCCN3C)[C@@H](C)CC)OC(C)=O)n2)cc1 ; *Nc1ccc(CS(=O)(=O)NC(=O)c2csc([C@@H](C[C@H](C(C)C)N(OCCCCC)C(=O)[C@@H](NC(=O)[C@H]3CCCCN3C)[C@@H](C)CC)OCC)n2)cc1]

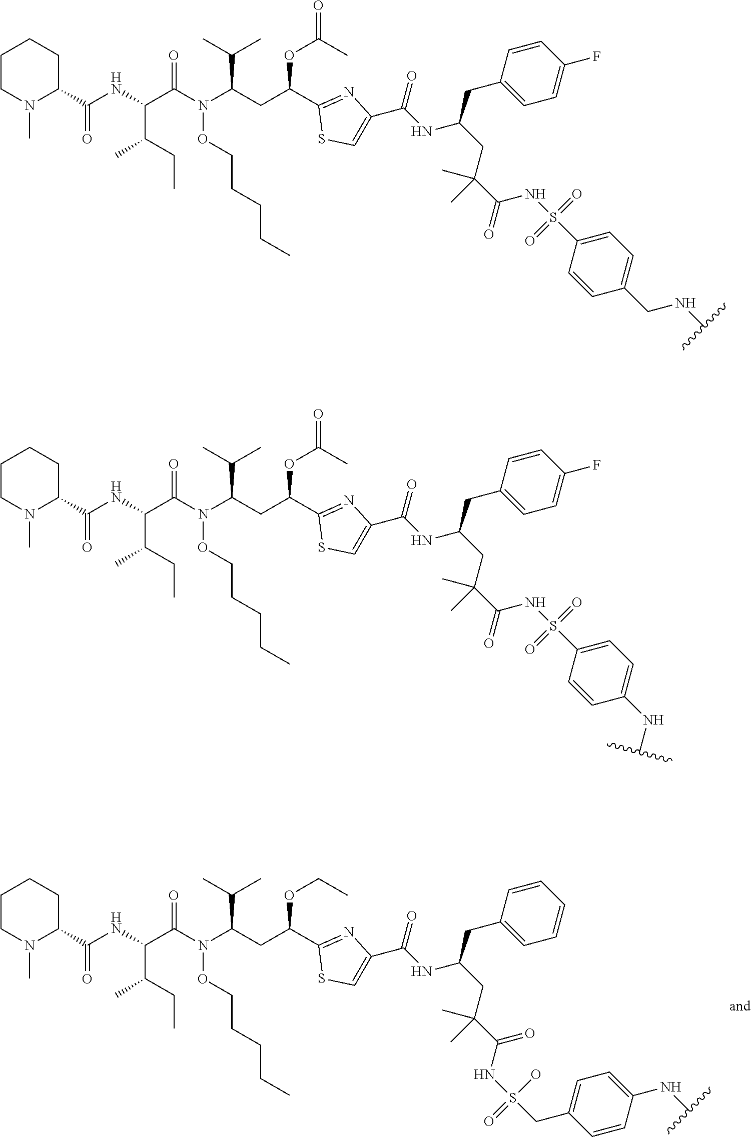

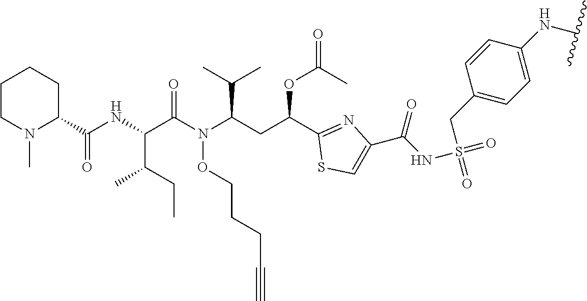 [SMILES: *Nc1ccc(CS(=O)(=O)NC(=O)c2csc([C@@H](C[C@H](C(C)C)N(OCCCC#C)C(=O)[C@@H](NC(=O)[C@H]3CCCCN3C)[C@@H](C)CC)OC(C)=O)n2)cc1]

37. The compound of claim 29, wherein BA or the antibody or antigen-binding fragment thereof is selected from the group consisting of anti-MUC16, anti-PSMA, anti-EGFRvIII, anti-HER2, and anti-MET.

38. The compound of claim 29, wherein BA or the antibody or antigen-binding fragment thereof is anti-PRLR or anti-STEAP2.

39. The compound of claim 29, wherein BA or the antibody or antigen-binding fragment thereof binds to an antigen selected from the group consisting of lipoproteins; alpha1-antitrypsin; a cytotoxic T-lymphocyte associated antigen (CTLA), such as CTLA-4 or CTLA4; vascular endothelial growth factor (VEGF); receptors for hormones or growth factors; protein A or D; fibroblast growth factor receptor 2 (FGFR2), EpCAM or Epcam, GD3, FLT3, PSCA, MUC1 or Muc1, MUC16 or Muc16, STEAP, STEAP2 or Steap-2, CEA, TENB2, EphA receptors, EphB receptors, folate receptor, FOLRI, mesothelin, cripto, alphavbeta6, VEGFR, EGFR, transferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80, CD81, CD103, CD105, CD134, CD137, CD138, CD152; erythropoietin; osteoinductive factors; immunotoxins; a bone morphogenetic protein (BMP); T-cell receptors; surface membrane proteins; integrins, such as CD11a, CD11b, CD11c, CD18, an ICAM, VLA-4 and VCAM; a tumor associated antigen such as AFP, ALK, B7H4, BAGE proteins, 0-catenin, brc-abl, BRCA1, BORIS, CA9 (carbonic anhydrase IX), caspase-8, CD123, CDK4, CLEC12 Å, c-kit, cMET, c-MET, MET, cyclin-B1, CYP1B1, EGFRvIII, endoglin, EphA2, ErbB2/Her2, ErbB3/Her3, ErbB4/Her4, ETV6-AML, Fra-1, FOLR1, GAGE proteins such as GAGE-1 and GAGE-2, GD2, GloboH, glypican-3, GM3, gp100, Her2 or HER2, HLA/B-raf, HLA/EBNA1, HLA/k-ras, HLA/MAGE-A3, hTERT, IGF1R, LGR5, LMP2, MAGE proteins such as MAGE-1, -2, -3, -4, -6, and -12, MART-1, ML-IAP, CA-125, MUM1, NA17, NGEP, NY-BR1, NY-BR62, NY-BR85, NY-ESO1, OX40, p15, p53, PAP, PAX3, PAX5, PCTA-1, PDGFR-a, PDGFR-0, PDGF-A, PDGF-B, PDGF-C, PDGF-D, PLAC1, PRLR, PRAME, PSGR, PSMA (FOLH1), RAGE proteins, Ras, RGS5, Rho, SART-1, SART-3, Steap-1, STn, survivin, TAG-72, TGF-β, TMPRSS2, Tn, TNFRSF17, TRP-1, TRP-2, tyrosinase, uroplakin-3, fragments of any of the above-listed polypeptides; cell-surface expressed antigens; molecules such as class A scavenger receptors including scavenger receptor A (SR-A), and other membrane proteins such as B7 family-related member including V-set and Ig domain-containing 4 (VSIG4), Colony stimulating factor 1 receptor (CSF1R), asialoglycoprotein receptor (ASGPR), and Amyloid beta precursor-like protein 2 (APLP-2); BCMA; SLAMF7; GPNMB; and UPK3 Å.

40. A compound having the structure of Formula I 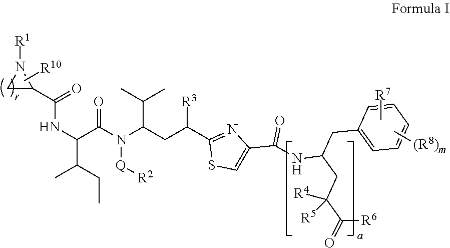
or a pharmaceutically acceptable salt thereof, wherein
R¹ is hydrogen, C₁-C₁₀ alkyl, a first N-terminal amino acid residue, —C₁-C₁₀ alkyl-NR^{3a}R^{3b}, or —C₁-C₁₀ alkyl-OH;
R³ is hydroxyl, —O—C₁-C₅ alkyl, —OC(O)C₁-C₅ alkyl, —OC(O)N(H)C₁-C₁₀ alkyl, —OC(O)N(H)C₁-C₁₀ alkyl-NR^{3a}R^{3b}, —NHC(O)C₁-C₅ alkyl, or —OC(O)N(H)(CH₂CH₂O)ₙC₁-C₁₀ alkyl-NR^{3a}R^{3b},
wherein R^{3a} and R^{3b} are independently in each instance, hydrogen, alkyl, alkenyl, alkynyl, cycloalkyl, aryl, heteroaryl, and acyl; wherein alkyl, alkenyl, alkynyl, cycloalkyl, aryl, heteroaryl, and acyl are optionally substituted;
R⁴ and R⁵ are, independently in each instance, hydrogen or C₁-C₅ alkyl;
R⁶ is —OH, —NHNH₂, —NHSO₂(CH₂)ₐ₁-aryl-(CH₂)ₐ₂NR^{6a}R^{6b},
wherein aryl is substituted or unsubstituted; and
R^{6a} and R^{6b} are independently in each instance, hydrogen, alkyl, alkenyl, alkynyl, cycloalkyl, aryl, heteroaryl, and acyl; wherein alkyl, alkenyl, alkynyl, cycloalkyl, aryl, heteroaryl, and acyl are optionally substituted;
R⁷ is, independently in each instance, hydrogen, —OH, halogen, or —NR^{7a}R^{7b},
wherein R^{7a} and R^{7b} are, independently in each instance, hydrogen, alkyl, alkenyl, alkynyl, cycloalkyl, aryl, heteroaryl, acyl, —C(O)CH₂OH, a first N-terminal amino acid residue, a first N-terminal peptide residue, and —CH₂CH₂NH₂; wherein alkyl, alkenyl, alkynyl, cycloalkyl, aryl, heteroaryl, and acyl are optionally substituted;
R⁸ is, independently in each instance, hydrogen, —NHR⁹, or halogen,
wherein R⁹ is hydrogen, —C₁-C₅ alkyl, or —C(O)C₁-C₅ alkyl; and
m is one or two;
R¹⁰, when present, is —C₁-C₅ alkyl;
Q is —CH₂— or —O— wherein
R² is alkyl, alkynyl, or a regioisomeric triazole;
wherein said regioisomeric triazole is unsubstituted or substituted with alkyl, alkenyl, alkynyl, cycloalkyl, aryl, heteroaryl, and acyl;
wherein n is an integer from one to ten;
wherein r is an integer from one to six;
wherein a, a1, and, a2 are, independently, zero or one; and
wherein T is not compound IVa, IVa′, IVb, IVc, IVd, IVe, IVf, IVg, IVh, IVj, IVk, IVl, IVm, IVn, IVo, IVp, IVq, IVr, IVs, IVt, IVu, IVvA, IVvB, IVw, IVx, IVy, Va, Va′, Vb, Vc, Vd, Ve, Vf, Vg, Vh, Vi, Vj, Vk, VIa, IVb, VIc, VId, VIe, VIf, VIg, VIh, VI, VIi, VII, VIII, IX, X, D-5a, D-5c, Tubulysin A-I, U-X, or Z, Pretubulysin D, or N¹⁴-desacetoxytubulysin H.

41.-67. (canceled)

68. A pharmaceutical composition comprising the compound of claim 1 and a pharmaceutically acceptable excipient, carrier, or diluent.

69. A method for treating cancer in a subject comprising administering to the subject of an effective treatment amount of the compound of claim 1.

70. A method for treating cancer in a subject comprising administering to the subject an effective treatment amount of the compound of claim 40.

71. A method for treating cancer in a subject comprising administering to the subject of an effective treatment amount of the compound of claim 1, wherein the cancer is selected from the group consisting of renal cell carcinoma, pancreatic carcinoma, head and neck cancer, prostate cancer, castrate-resistant prostrate cancer, malignant gliomas, osteosarcoma, colorectal cancer, gastric cancer, mesothelioma, malignant mesothelioma, multiple myeloma, ovarian cancer, lung cancer, small cell lung cancer, non-small cell lung cancer, synovial sarcoma, thyroid cancer, breast cancer, PRLR positive (PRLR+) breast cancer, melanoma, acute myelogenous leukemia, adult T-cell leukemia, astrocytomas, bladder cancer, cervical cancer, cholangiocarcinoma, endometrial cancer, esophageal cancer, glioblastomata, Kaposi's sarcoma, kidney cancer, leiomyosarcomas, liver cancer, lymphomas, MFH/fibrosarcoma, nasopharyngeal cancer, rhabdomyosarcoma, colon cancer, stomach cancer, uterine cancer, residual cancer, and Wilms' tumor.

72. A method for treating cancer in a subject comprising administering to the subject of an effective treatment amount of the compound of claim 40, wherein the cancer is selected from the group consisting of renal cell carcinoma, pancreatic carcinoma, head and neck cancer, prostate cancer, castrate-resistant prostrate cancer, malignant gliomas, osteosarcoma, colorectal cancer, gastric cancer, mesothelioma, malignant mesothelioma, multiple myeloma, ovarian cancer, lung cancer, small cell lung cancer, non-small cell lung cancer, synovial sarcoma, thyroid cancer, breast cancer, PRLR positive (PRLR+) breast cancer, melanoma, acute myelogenous leukemia, adult T-cell leukemia, astrocytomas, bladder cancer, cervical cancer, cholangiocarcinoma, endometrial cancer, esophageal cancer, glioblastomata, Kaposi's sarcoma, kidney cancer, leiomyosarcomas, liver cancer, lymphomas, MFH/fibrosarcoma, nasopharyngeal cancer, rhabdomyosarcoma, colon cancer, stomach cancer, uterine cancer, residual cancer, and Wilms' tumor.

73. A method for treating tumors that express an antigen selected from the group consisting of PRLR and STEAP2.

74. A linker-payload having the formula
L-T
or a pharmaceutically acceptable salt thereof, wherein
L is a linker covalently bound to T;
T is 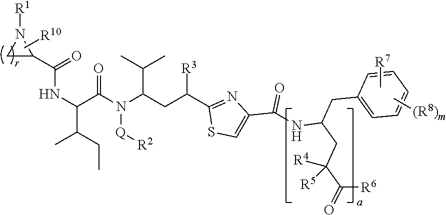
wherein
R¹ is a bond, hydrogen, C₁-C₁₀ alkyl, a first N-terminal amino acid residue, a first amino acid residue, —C₁-C₁₀ alkyl-NR^{3a}R^{3b}, or —C₁-C₁₀ alkyl-OH;
R³ is hydroxyl, —O—, —O—C₁-C₅ alkyl, —OC(O)C₁-C₅ alkyl, —OC(O)N(H)C₁-C₁₀ alkyl, —OC(O)N(H)C₁-C₁₀ alkyl-NR^{3a}R^{3b}, —NHC(O)C₁-C₅ alkyl, or —OC(O)N(H)(CH₂CH₂O)ₙC₁-C₁₀ alkyl-NR^{3a}R^{3b},
wherein R^{3a} and R^{3b} are independently in each instance, a bond, hydrogen, alkyl, alkenyl, alkynyl, cycloalkyl, aryl, heteroaryl, and acyl; wherein alkyl, alkenyl, alkynyl, cycloalkyl, aryl, heteroaryl, and acyl are optionally substituted;
R⁴ and R⁵ are, independently in each instance, hydrogen or C₁-C₅ alkyl;
R⁶ is —OH, —O—, —NHNH₂, —NHNH—, —NHSO₂(CH₂)ₐ₁-aryl-(CH₂)ₐ₂NR^{6a}R^{6b},
wherein aryl is substituted or unsubstituted; and
R^{6a} and R^{6b} are independently in each instance, a bond, hydrogen, alkyl, alkenyl, alkynyl, cycloalkyl, aryl, heteroaryl, and acyl; wherein alkyl, alkenyl, alkynyl, cycloalkyl, aryl, heteroaryl, and acyl are optionally substituted;
R⁷ is, independently in each instance, hydrogen, —OH, —O—, halogen, or —NR^{7a}R^{7b},
wherein R^{7a} and R^{7b} are, independently in each instance, a bond, hydrogen, alkyl, alkenyl, alkynyl, cycloalkyl, aryl, heteroaryl, acyl, —C(O)CH₂OH, —C(O)CH₂O—, a first N-terminal amino acid residue, a first amino acid residue, a first N-terminal peptide residue, a first peptide residue, —CH₂CH₂NH₂, and —CH₂CH₂NH—; wherein alkyl, alkenyl, alkynyl, cycloalkyl, aryl, heteroaryl, and acyl are optionally substituted;
R⁸ is, independently in each instance, hydrogen, —NHR⁹, or halogen,
wherein R⁹ is hydrogen, —C₁-C₅ alkyl, or —C(O)C₁-C₅ alkyl; and
m is one or two;
R¹⁰, when present, is —C₁-C₅ alkyl;
Q is —CH₂— or —O— wherein
R² is alkyl, alkylene, alkynyl, alkynylene, a regioisomeric triazole, a regioisomeric triazolylene;
wherein said regioisomeric triazole or regioisomeric triazolylene is unsubstituted or substituted with alkyl, alkenyl, alkynyl, cycloalkyl, aryl, heteroaryl, or acyl;
wherein n is an integer from one to ten;
wherein r is an integer from one to six;
wherein a, a1, and, a2 are, independently, zero or one; and
wherein the linker-payload is not LP1-IVa, LP2-Va, LP3-IVd, LP4-Ve, LP5-IVd, LP6-Vb, LP7-IVd, LP9-IVvB, LP10-VIh, LP11-IVvB, LP12-VIi, LP13-Ve, LP14-Ve, LP15-VIh, LP16-Ve, LP17-Ve, LP18-Ve, LP19-Ve, LP20-Ve, LP21-Ve, LP22-Ve, LP23-Vb, LP24-Vb, LP25-Ve, and LP26-Ve, or a pharmaceutically acceptable salt thereof.

75.-100. (canceled)

101. A pharmaceutical composition comprising the compound of claim 40 and a pharmaceutically acceptable excipient, carrier, or diluent.

102. A method for treating cancer in a subject comprising administering to the subject of an effective treatment amount of the pharmaceutical composition of claim 68.

103. A method for treating cancer in a subject comprising administering to the subject of an effective treatment amount of the pharmaceutical composition of claim 101.

104. A method for treating cancer in a subject comprising administering to the subject of an effective treatment amount of the pharmaceutical composition of claim 68, wherein the cancer is selected from the group consisting of renal cell carcinoma, pancreatic carcinoma, head and neck cancer, prostate cancer, castrate-resistant prostrate cancer, malignant gliomas, osteosarcoma, colorectal cancer, gastric cancer, mesothelioma, malignant mesothelioma, multiple myeloma, ovarian cancer, lung cancer, small cell lung cancer, non-small cell lung cancer, synovial sarcoma, thyroid cancer, breast cancer, PRLR positive (PRLR+) breast cancer, melanoma, acute myelogenous leukemia, adult T-cell leukemia, astrocytomas, bladder cancer, cervical cancer, cholangiocarcinoma, endometrial cancer, esophageal cancer, glioblastomata, Kaposi's sarcoma, kidney cancer, leiomyosarcomas, liver cancer, lymphomas, MFH/fibrosarcoma, nasopharyngeal cancer, rhabdomyosarcoma, colon cancer, stomach cancer, uterine cancer, residual cancer, and Wilms' tumor.

105. A method for treating cancer in a subject comprising administering to the subject of an effective treatment amount of the pharmaceutical composition of claim 101, wherein the cancer is selected from the group consisting of renal cell carcinoma, pancreatic carcinoma, head and neck cancer, prostate cancer, castrate-resistant prostrate cancer, malignant gliomas, osteosarcoma, colorectal cancer, gastric cancer, mesothelioma, malignant mesothelioma, multiple myeloma, ovarian cancer, lung cancer, small cell lung cancer, non-small cell lung cancer, synovial sarcoma, thyroid cancer, breast cancer, PRLR positive (PRLR+) breast cancer, melanoma, acute myelogenous leukemia, adult T-cell leukemia, astrocytomas, bladder cancer, cervical cancer, cholangiocarcinoma, endometrial cancer, esophageal cancer, glioblastomata, Kaposi's sarcoma, kidney cancer, leiomyosarcomas, liver cancer, lymphomas, MFH/fibrosarcoma, nasopharyngeal cancer, rhabdomyosarcoma, colon cancer, stomach cancer, uterine cancer, residual cancer, and Wilms' tumor.
